(19) United States
(12) Patent Application Publication
(10) Pub. No.: US 2025/0122301 A1
(43) Pub. Date: Apr. 17, 2025

(54) CD25 ANTIBODIES

(71) Applicant: iBio, Inc., San Diego, CA (US)

(72) Inventors: Bing Li, San Diego, CA (US); Matthew P. Greving, San Diego, CA (US); Phung Tu Gip, San Diego, CA (US); Matt Lundberg, San Diego, CA (US); Mohan Srinivasan, San Diego, CA (US)

(21) Appl. No.: 18/968,640

(22) Filed: Dec. 4, 2024

Related U.S. Application Data
(63) Continuation of application No. 17/320,125, filed on May 13, 2021, now Pat. No. 12,215,163.
Application No. 17/320,125 is a continuation of application No. PCT/US2019/061552, filed on Nov. 14, 2019.
(60) Provisional application No. 62/767,405, filed on Nov. 14, 2018.

(51) Int. Cl. C07K 16/28; A61P 37/06; C12N 15/63

(52) U.S. Cl. CPC C07K 16/2896; A61P 37/06; C12N 15/63; C07K 2317/24; C07K 2317/565; C07K 2317/92

(57) ABSTRACT

Provided herein are antibodies that specifically bind to CD25. Also provided herein are methods of making the antibodies described, and methods of use thereof. For example, the CD25 antibodies may be used therapeutically to treat cancer or autoimmune diseases, and in certain aspects: disrupt the trimerization of the beta, gamma, and alpha (CD25) chains of the IL-2 receptor, bind to a different epitope than to which Daclizumab or Baciliximab bind, exhibit a higher affinity of binding to CD25 at a pH lower than 7.4, when compared to the affinity of binding to CD25 at a pH of 7.4, and/or exhibit a higher affinity of binding to CD25 at a pH of about 6.5.

### CROSS-REFERENCE TO RELATED APPLICATIONS

This application is a continuation of U.S. application Ser. No. 17/320,125 filed May 13, 2021, which is a continuation of International Application No. PCT/US2019/061552, filed Nov. 14, 2019, which claims the priority benefit of U.S. Provisional Application No. 62/767,405, filed on Nov. 14, 2018, the entire contents of which are hereby incorporated by reference in their entirety for all purposes.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 3, 2024, is named “IBIO1029CON.xml” and is 1,316,007 bytes in size. The sequence listing contained in this .XML file is part of the specification and is hereby incorporated by reference herein in its entirety.

### BACKGROUND

The CD25 protein is the alpha chain of interleukin-2 (IL-2) receptor and is a transmembrane protein present on regulatory T cells, and activated T cells. In a normal state, regulatory T cells constitutively express CD25 and act to suppress the expansion of effector cells. Regulatory T cells maintain the healthy state and inhibit effector T cells from reacting against self antigens or over-reacting to foreign antigens. In a normal, protective immune response, effector T cells multiply after contact with foreign antigen and overcome inhibition by regulatory T cells. In case of proliferative diseases, however cancer cells disable the healthy immune response by increasing the amount of regulatory T cells and thereby limiting the generation of effector T cells against them. Additional molecular tools to alter the proliferation of CD25-expressing regulatory T cells are needed, for example to dampen the immune system for use in cancer therapies or to upregulate the immune system for use in autoimmune diseases; provided herein are such tools.

### SUMMARY

Provided herein are antibodies that specifically bind to CD25 (anti-CD25 antibodies, interchangeably referred to herein as CD25 antibodies). The antibodies may be human, chimeric or humanized. Also provided herein are methods of use and methods of making the antibodies described. For example, the CD25 antibodies may be used therapeutically to treat cancer, comprising administering to a subject in need thereof an antibody or a pharmaceutical composition thereof. Also provided are methods of producing the CD25 antibodies described herein.

In one aspect, provided herein is a monoclonal CD25 antibody which binds to human CD25, and possesses at least one, at least two, at least three, at least four, at least five, or at lease six of the following characteristics:
a. the antibody does not disrupt the binding of the IL-2 ligand to the alpha chain of the IL-2 receptor (CD25), and binds to a different epitope than to which 7G7B6 binds;
b. the antibody does not disrupt the binding of the IL-2 ligand to the alpha chain of the IL-2 receptor (CD25), but does disrupt the trimerization of the beta, gamma, and alpha (CD25) chains of the IL-2 receptor;
c. the antibody disrupts the binding of the IL-2 ligand to the alpha, beta, and/or gamma chains of the IL-2 receptor, and binds to a different epitope than to which daclizumab or baciliximab bind;
d. the antibody exhibits a higher affinity of binding to CD25 at pH lower than 7.4, when compared to the affinity of binding to CD25 at a pH of 7.4;
e. the antibody comprises the amino acid sequence of any one of the variable heavy chains presented in Tables 1A, 1C, 1E, 1G, 1I, 1K, 2A, 2B, 2C, 4A, and 5A or FIGS. 3A, 3B and 5, humanized versions thereof, or an amino acid sequence comprising at least a 80%, at least a 85%, at least a 90%, or at least a 95% sequence identity thereto;
f. the antibody comprises the amino acid sequence of any one of the variable light chains presented in Tables 1B, 1D, 1F, 1H, 1J, 1L, 3A, 3B, 3C, 4B, and 5B or FIGS. 4A, 4B and 6, humanized versions thereof, or an amino acid sequence comprising at least a 80%, at least a 85%, at least a 90%, or at least a 95% sequence identity thereto;
g. the VH of the antibody comprises any one of the amino acid sequence of CDRH1, CDRH2, and CDRH3 as presented in Tables 1A, 1C, 1E, 1G, 1I, 1K, 2A, 2B, 2C, 4A, 5A, and 6 or contained in the sequences presented in FIGS. 3A, 3B and 5;
h. the VL of the CD25 antibody comprises any one of the amino acid sequence of CDRL1, CDRL2, and CDRL3 as presented in Tables 1B, 1D, 1F, 1H, 1J, 1L, 3A, 3B, 3C, 4B, 5B and 7, or contained in the sequences presented in FIGS. 4A, 4B and 6; and
i. the antibody comprises the amino acid sequence of CDRH1, CDRH2, and CDRH3 of any one of the combinations presented in Table 6 and the amino acid sequence of CDRL1, CDRL2, and CDRL3 of any one of the combinations presented in Table 7.

In some embodiments, the VH of the antibody comprises the amino acid sequence of CDRH1, CDRH2, and CDRH3 combinations as presented in Tables 1A, 1C, 1E, 1G, 1I, 1K, 2A, 2B, 2C, 4A, 5A, or 6 or contained in the sequences presented in FIG. 3A, 3B or 5.

In some embodiments, the VL of the CD25 antibody comprises the amino acid sequence of CDRL1, CDRL2, and CDRL3 combinations as presented in Tables 1B, 1D, 1F, 1H, 1J, 1L, 3A, 3B, 3C, 4B, 5B, or 7 or contained in the sequences presented in FIG. 4A, 4B or 6.

In some embodiments, the CD25 antibody comprises the amino acid sequence of CDRH1, CDRH2, and CDRH3 of any one of the combinations presented in Table 6.

In some embodiments, the CD25 antibody comprises the amino acid sequence of CDRL1, CDRL2, and CDRL3 of any one of the combinations presented in Table 7.

In some embodiments the antibody is a human antibody. In some embodiments the antibody is a humanized antibody. In some embodiments the antibody is a chimeric antibody. In some embodiments the antibody is an antibody fragment. In some embodiments the antibody also binds cynomologous monkey CD25.

In another aspect, provided herein is a method of treating a subject in need thereof comprising administering to the subject a therapeutically effective amount of any one of the antibodies or pharmaceutical compositions described herein. In some embodiments, provided herein is a method of depleting the number of regulatory T cells in a subject comprising administering to the subject a therapeutically effective amount of any one of the antibodies or pharmaceutical compositions described herein. In some embodiments, the subject suffers from cancer; in other embodiments, the subject suffers from an autoimmune-related disease or disorder.

In another aspect, provided herein is a method of depleting the number of regulatory T cells in a sample comprising peripheral blood mononuclear cells comprising contacting the sample with any one of the antibodies described herein.

In related aspects, provided herein are pharmaceutical compositions or kits comprising any one or more of the antibodies described herein, nucleic acid sequences encoding any of the antibodies described herein, vectors comprising the nucleic acid and phage expressing any of the antibodies described herein.

All of the above features described herein (including any accompanying claims, abstract and drawings), and/or all of the steps of any method or process so disclosed, may be combined in any combination, except combinations where at least some of such features and/or steps are mutually exclusive.

### DESCRIPTION OF THE DRAWINGS

The present application can be understood by reference to the following description taken in conjunction with the accompanying figures.

FIG. 1 is an exemplary description of the desired characteristics of one of the class of CD25 antibodies described herein: CD25 antibodies that block the alpha chain of the IL-2 receptor (CD25) only, do not block binding of IL-2, do not disrupt signal transduction mediated by IL-2, and leads to depletion of Tregs. Data are collected with Fab clones, but as provided herein, in some embodiments, the CD25 antibodies are full length antibodies, i.e. human IgG1 antibodies.

FIG. 2 is an exemplary workflow for CD25 antibody discovery, in vitro testing, and in vivo testing.

FIG. 3A and FIG. 3B depict variable heavy chain (VH) amino acid sequences of exemplary CD25 antibodies (SEQ ID NOs: 61-107, 1501 and 432-460) of the disclosure.

FIG. 4A and FIG. 4B depict variable light chain (VL) amino acid sequences of exemplary CD25 antibodies (SEQ ID NOs: 703, 705, 707, 709, 715, 717, 722, 724, 725, 727, 728, 739, 742, 745, 747, 749-752, 754-756, 759, 762, 765, 767, 769, 776, 780, 781, 783, 785, 787, 789, 791, 794, 797, 799, 801, 803, 804, 806, 809-811, 813, 815, 818, 824, 827, 829, 835, 841, 842, 844, 846, 848, 849, 851, 853, 875-880, 882, 884, 886, 888, 892, 897, 901-903, 905-908, 914, 917, 919-922) of the disclosure.

FIG. 5 depicts VH amino acid sequences of exemplary CD25 antibodies (SEQ ID NOs: 868, 869, 1222, 1229-1238, 1316-1318 and 1327-1335) of the disclosure.

FIG. 6 depicts VL amino acid sequences of exemplary CD25 antibodies (SEQ ID NOs: 34, 701, 712, 735, 736, 737, 775, 777, 778, 795, 822, 823, 825, 838, 857, 859, 860, 861, 864, 895 and 912) of the disclosure.

FIG. 7 depicts the identification of non IL-2 blockers, using a cross block assay, using a biosensor.

FIG. 8 depicts phage express Fabs of the disclosure that compete with IL-2 non-blocking antibody, 7G7B6, using a biosensor.

FIG. 9 shows that pSTAT5 levels are IL-2 dose dependent and inhibited with an IL-2 blocking antibody, Daclizumab.

FIG. 10 depicts the effect of the D5 Fab of the disclosure on pSTAT5 levels. The data indicate that the D5 Fab is a partial IL-2 blocker.

FIG. 11 depicts the effect of the D5 Fab at different concentrations on pSTAT5 levels, relative to maximum IL-2 pSTAT5 levels.

FIG. 12 depicts differences in pSTAT5 levels between several Fab clones at 1, 2 and 5 ug/mL compared to the controls (IL-2 only, Daclizumab (Dac) and 7G7B6.

FIGS. 13A-13C depict differences in Kd and Koff rates for several Fab clones that were reformatted to human IgG1 antibodies (FIG. 13A). Several reformatted clones have better affinity and Koff rates than commercially available antibodies 7G7B6, Daclizumab and Basiliximab (FIG. 13B, FIG. 13C).

FIGS. 14A-14D show representative data from epitope bining cross competition assays against IL-2 and commercially available antibodies 7G7B6, Daclizumab and Basiliximab, using Fab clones that were reformatted to human IgG1 antibodies. Antibodies had different cross blocking profiles.

FIGS. 15A-15B depicts specific binding of human IgG1 reformatted Fab on CD25+ cell lines SUDHL-1 and HEK IL-2 reporter cells (FIG. 15A) and no binding on CD25-cell line SUDHL-2 (FIG. 15B).

FIGS. 16A-16B show representative dose reponse curves of human IgG1 reformatted Fab clones on CD25+ cell line SUDHL-1 compared to commercially available antibodies 7G7B6, Daclizumab and Basiliximab (FIG. 16A). Several clones show better EC50 values than IL-2 non-blocker 7G7B6 (FIG. 16B).

FIG. 17. show representative data of human IgG1 reformatted Fab clones that bind to recombinant cynomolgus monkey CD25 protein (25 nM). Majority of clones bind in a dose dependent manner (starting at 25 nM with 3 fold dilutions).

FIGS. 18A-18B. show differences in pSTAT5 levels across several human IgG1 reformatted Fab clones at 5 ug/ml, compared to IL-2 levels at 0.1 ng/mL (FIG. 18A). FIG. 18B shows pSTAT5 levels with IL-2 dose response curve (starting at 10 ng/ml with 10 fold dilutions) with 5 ug/mL of each antibody. Some clones were shown to be better IL-2 blocker and IL-2 nonblockers than commercially available antibodies, 7G7B6, Daclizumab and Basiliximab at 0.1 ng/mL.

FIGS. 19A-19B. show representative data of human IgG1 reformatted Fab clones at 20 ug/mL, that elicited cell killing in an ADCC assay using PBMCs as effector cells and SUHL-1 cells as targets (FIG. 19A). Several clones showed higher ADCC than 7G7B6 starting at 10 ug/mL with 5 fold dilutions of antibody (FIG. 19B).

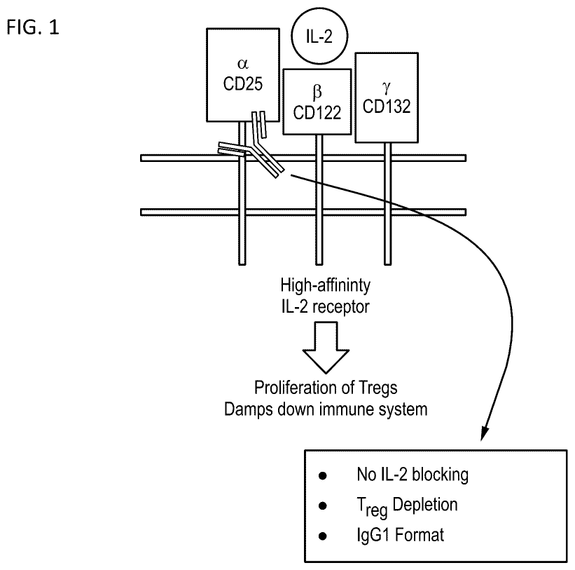

### DETAILED DESCRIPTION

Provided herein are antibodies that specifically bind to CD25. The antibodies may be human, chimeric or humanized. Also provided herein are methods of use and methods of making the antibodies described. For example, the CD25 antibodies may be used therapeutically to treat cancer, comprising administering to a subject in need thereof an antibody or a pharmaceutical composition thereof. Also provided are methods of producing the CD25 antibodies described herein.

### Definitions

Unless defined otherwise herein, all technical and scientific terms used herein have the same meaning as commonly understood by one of ordinary skill in the art to which this invention belongs. Any methods and materials similar or equivalent to those described herein can be used in the practice or testing of the present invention, exemplary methods and materials are described.

The headings provided herein are not limitations of the various aspects or embodiments of the invention. Accordingly, the terms defined immediately below are more fully defined by reference to the specification as a whole.

Numeric ranges are inclusive of the numbers defining the range.

The term antibody, as used herein, includes but is not limited to a monoclonal antibody, polyclonal antibody, human antibody, humanized antibody, non-human antibody, chimeric antibody, monovalent antibody and antigen-binding fragments of the antibody (e.g Fab fragment, a Fab′2 fragment, or a scFV). Also provided herein are antibody-drug conjugates, bispecific antibodies, and multispecific antibodies that exhibit specificity for CD25. A non-human antibody (e.g. a mouse antibody) may be “humanized” using conventional techniques (e.g. by introducing changes in the framework region, while retaining mouse CDRs).

The terms “polynucleotide” and “nucleic acid” are used interchangeably herein, and refer to a polymeric form of nucleotides of any length, which may be ribonucleotides or deoxyribonucleotides. The terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms encompass nucleic acids containing known analogues of natural nucleotides and having similar binding properties, and are metabolized in a manner similar to naturally-occurring nucleotides, unless specifically limited or stated otherwise.

When a nucleic acid or amino acid sequence is said to have a certain percent “sequence identity” or “identity” or is a certain percent “identical” to another nucleic acid or amino acid sequence, that percentage of bases or amino acids are the same, and in the same relative position, when the sequences are aligned, when comparing the two sequences.

The terms “individual,” “subject,” and “patient” are used interchangeably herein and refer to any subject for whom treatment or therapy is desired. The subject may be a mammalian subject. Mammalian subjects include, e.g., humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate. In some embodiments, the subject is a companion animal (e.g. cats, dogs).

### Antibodies

Provided herein are antibodies that specifically bind to CD25. Such antibodies are capable of binding to the CD25 antigen either alone or associated with other molecules to form high affinity IL-2 receptors which is present on regulatory T cells.

In some embodiments, the CD25 antibody is a humanized antibody that specifically binds to CD25.

In some embodiments, the CD25 antibody is a chimeric antibody that for example a mouse-human chimeric antibody, e.g. an antibody that comprises mouse variable domains, and a human constant region.

A CD25 antibody of the disclosure can be any of a human IgA, IgD, IgE, IgG, or IgM antibody. The IgA antibody can be an IgA1 or an IgA2 antibody. The IgG antibody can be an IgG1, IgG2, IgG2a, IgG2b, IgG3 or IgG4 antibody. A combination of any of these antibodies can also be made and used. In some embodiments, the constant region is of the IgG type, e.g. of the human IgG type. In some embodiments, the constant region is of the IgG1 type, e.g. of the human IgG1 type.

In some embodiments, the CD25 antibody exhibits cross reactivity to more than one species, for example specifically binds to both human CD25 and non-human CD25, for example specifically binds to both human CD25 and cynomolgus monkey CD25.

The KD (affinity constant) of the antibodies provided herein range from about 10\hat{ }-5 to about 10\hat{ }-14 nM. In some embodiments the KD of the antibodies provided herein range from about 10\hat{ }-8 to about 10\hat{ }-12 nM. In exemplary embodiments, the KD of a CD25 antibody is at least about 10\hat{ }-5 nM, about 10\hat{ }-6 nM, about 10\hat{ }-7 nM nM, about 10\hat{ }-8 nM, about 10\hat{ }-9 nM, about 10\hat{ }-10 nM, about 10\hat{ }-11 nM, about 10\hat{ }-12 nM, about 10\hat{ }-13 nM, or even about 10\hat{ }-14 nM.

The Kd (off-rate constant) of the antibodies provided herein range from about 10\hat{ }-2 to about 10\hat{ }-6 l/s.

In some embodiments, the CD25 antibody exhibits the same affinity (KD) for the CD25 antigen at both physiological pH (about 7.4) and non-physiological pH. In some embodiments, the CD25 antibody exhibits the same off-rate (Kd) for the CD25 antigen at both physiological pH (about 7.4) and non-physiological pH.

In some embodiments, the CD25 antibody exhibits different affinities (different KD) for the CD25 antigen at physiological pH (about 7.4) and non-physiological pH. In some embodiments, the CD25 antibody exhibits different off-rate constants (different Kd) for the CD25 antigen at physiological pH (about 7.4) and non-physiological pH.

In some embodiments, the CD25 antibody exhibits a lower affinity (higher KD) for the CD25 antigen at physiological pH (about 7.4) than at a pH that is lower than physiological pH, for example when the pH is 7.3, 7.2, 7.1, 7.0, 6.9, 6.8, 6.7, 6.6, 6.5, 6.4, 6.3, 6.2, 6.1, or lower. In exemplary embodiments, the antibody exhibits a higher affinity for the CD25 antigen at a pH of about 6.5, when compared to the affinity at a pH of about 7.4. In some embodiments, such antibodies are useful for retaining activity, or exhibiting enhanced activity in an acidic environment, a hypoxic environment, for example a tumor microenvironment.

In some embodiments, the antibody is a non IL-2-blocking antibody (a non IL-2 blocker)—that is, the binding of the antibody to CD25 does not disrupt or prevent binding of the IL-2 ligand to CD25 (the IL-2 alpha chain), and does not affect IL-2 mediated signal transduction, e.g. signaling through the IL-2/JAK3/STAT-5 signaling pathway. In some embodiments, the antibody does not disrupt the binding of IL-2 ligand to CD25 (IL-2 alpha chain), and binds to a different epitope than where the 7G7B6 antibody binds. In some embodiments, the antibody does not disrupt the binding of the IL-2 ligand to CD25 (IL-2 alpha chain), but does disrupt the trimerization of the beta, gamma, and alpha (CD25) chains of the IL-2 receptor.

In some embodiments, the antibody is an IL-2 blocking antibody (IL-2 blocker), e.g., the antibody disrupts or prevents binding of the IL-2 ligand to the alpha, beta, and/or gamma chains of the receptor, and decreases or inhibits IL-2 mediated signal transduction. In certain embodiments, the antibody disrupts or prevents binding of the IL-2 ligand to CD25. In some embodiments, the antibody disrupts or prevents the binding of the IL-2 ligand to CD25, and binds to a different epitope than to which either daclizumab or baciliximab bind.

In some embodiments, the CD25 antibody is a partially blocking antibody (partial IL-2 blocker), and partially, but not completely, disrupts binding of the IL-2 ligand to the alpha, beta, and/or gamma chains of the IL-2 receptor (CD25), and/or partially, but not completely decreases IL-2 mediated signal transduction.

In some embodiments, the CD25 antibody disrupts or prevents heterotrimerization of the alpha, beta, and gamma IL-2 chains. In some embodiments, the antibody does not block binding of the IL-2 ligand with CD25, but does disrupt or prevent heterotrimerization of the alpha, beta, and gamma IL-2 chains. In certain embodiments, the antibody selectively binds to regulatory T cells. In other embodiments, the antibody selectively binds T effector cells.

In some embodiments, binding of the CD25 antibody leads to the depletion of regulatory T cells (Tregs), while allowing for expansion of effector T cells (Teff).

In some embodiments, the antibody binds to CD25 in the trans orientation. In other embodiments the antibody binds to CD25 in the cis orientation. In still further embodiments, the antibody is capable of binding to CD25 in either the cis or the trans configuration.

In some embodiments, the CD25 antibody exhibits greater binding affinity to CD25, as compared to the binding of 7G7B6 (anti-human CD25 with mouse IgG2a Fc receptor; IL-2 non blocker; BioXcell) to CD25.

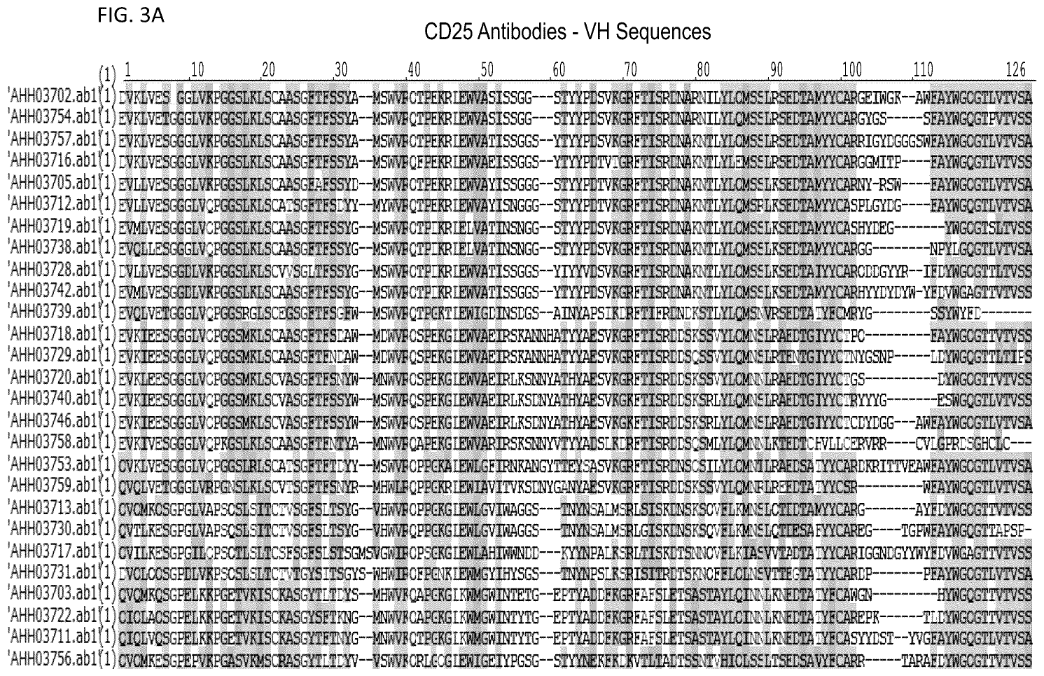

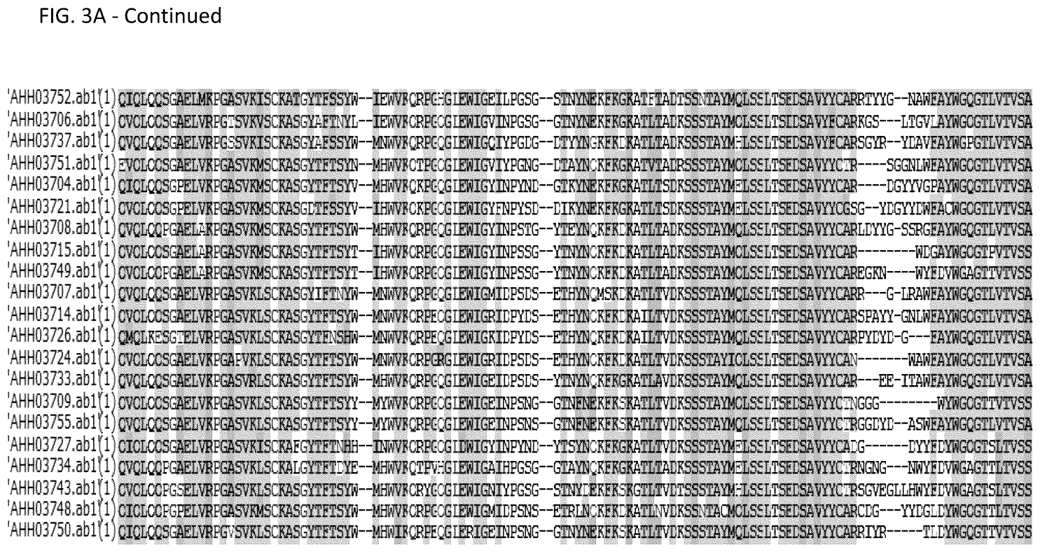

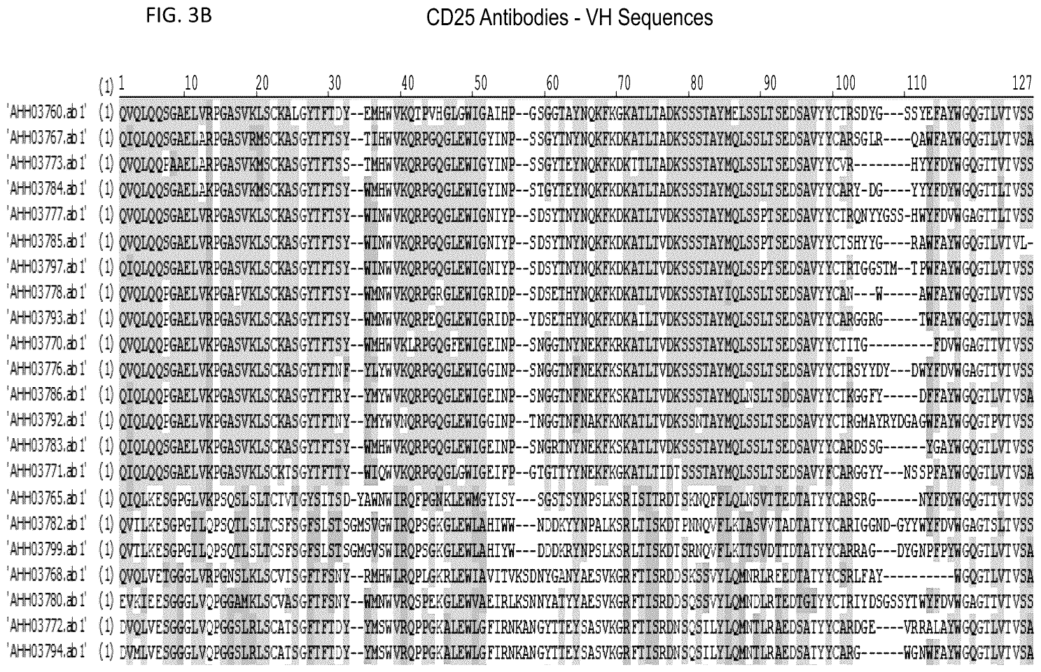

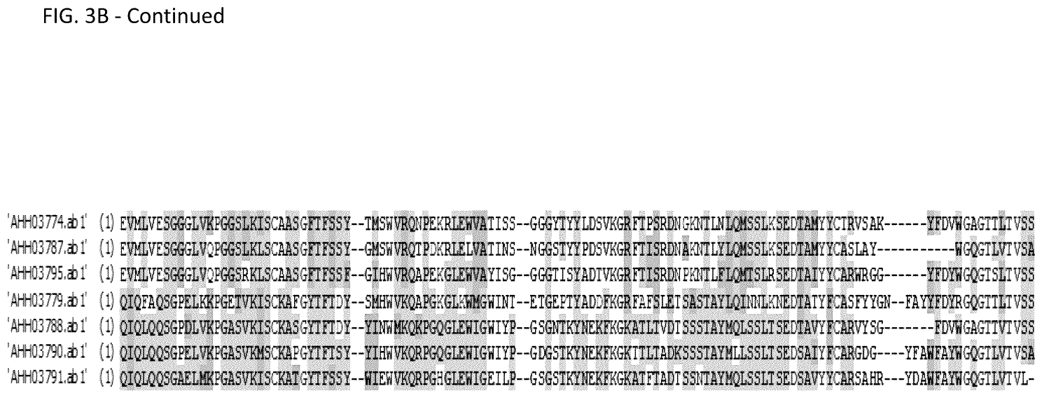

Tables 1A-1L, 2A-2C, 3A-3C, 4A-4B, 5A-5B, 6, and 7 and FIGS. 3A, 3B, 4A, 4B, 5, and 6 provide exemplary sequences for the CD25 antibodies described herein. It is noted that the complementarity determining region (CDR) and framework (FR) sequences shown are based on the IMGT convention for antibody annotation. However a skilled artisan can determine other articulations of CDR and framework sequences based on the presented VH and VL sequences, using other algorithms/conventions for antibody annotation, such as Kabat and Chothia. Accordingly the CDRs and FR sequences of the disclosure are not limited to those exemplary CDRs and FR sequences annotated in the below tables, but rather the CDRs as would be understood and determined by a skilled artisan, given the sequence of the variable region.

TABLE 1A
D5 VH Sequences

D5 VH - Nucleic Acid
catgtgcaactgcagcagtctggagctgagctggtaaggcctgggacttc
agtgaagatatcctgcaaggcttctggctacaccttcactaactactggc
taggttgggtaaagcagaggcctggacatggacttgagtggattggagat
atttaccctggaggtggttatactaactacaatgagaagttcaagggcaa
ggccacactgactgcagacacatcctccagcactgcctacatgcagctca
gtagcctgacatctgaggactctgctgtctatttctgtgcaagagttact
ccggcttcctggggccaaggcaccagtctcacagtctcctcgg (SEQ
ID NO: 1)
D5 VH - Amino Acid
HVQLQQSGAELVRPGTSVKISCKASGYTFTNYWLGWVKQRPGHGLEWIGD
IYPGGGYTNYNEKFKGKATLTADTSSSTAYMQLSSLTSEDSAVYFCARVT
PASWGQGTSLTVSS (SEQ ID NO: 2)
D5 VH CDR1 - IMGT
ggctacaccttcactaactactgg (SEQ ID NO: 3)
GYTFTNYW (SEQ ID NO: 4)
D5 VH CDR2 - IMGT
atttaccctggaggtggttatact (SEQ ID NO: 5)
IYPGGGYT (SEQ ID NO: 6)
D5 VH CDR3 - IMGT
gcaagagttactccggcttcc (SEQ ID NO: 7)
ARVTPAS (SEQ ID NO: 8)

TABLE 1B
D5 VL Sequences

D5 VL - Nucleic Acid
gatattgtgatgacccagtctcaaaaattcatgtccacatcagtaggaga
cagggtcagcgtcacctgcaaggccagtcagaatgtgggtactaatgtag
cctggtatcaacagaaaccagggcaatctcctaaagcactgatttactcg
gcatcctaccggtacagtggagtccctgatcgcttcacaggcagtggatc
tgggacagatttcactctcaccatcagcaatgtgcagtctgaagacttgg
cagagtatttctgtcagcaatataacagctatccgtacacgttcggaggg
gggaccaagctggaaatgaaac (SEQ ID NO: 9)
D5 VL - Amino Acid
DIVMTQSQKFMSTSVGDRVSVTCKASQNVGTNVAWYQQKPGQSPKALIYS
ASYRYSGVPDRFTGSGSGTDFTLTISNVQSEDLAEYFCQQYNSYPYTFGG
GTKLEMK (SEQ ID NO: 10)
D5 VL - CDR1-IMGT
cagaatgtgggtactaat (SEQ ID NO: 11)
QNVGTN (SEQ ID NO: 12)
D5 VL - CDR2-IMGT
tcggcatcc
SAS (SEQ ID NO: 13)
D5 VL - CDR3-IMGT
cagcaatataacagctatccgtacacg (SEQ ID NO: 14)
QQYNSYPYT (SEQ ID NO: 15)

TABLE 1C
D11 VH Sequences

D11 VH-Nucleic Acid
cagatccaactgcagcagcctggggctgagctggtgaggcctggggtttc
actgaaaatttcctgcaagggttctggctacacattcactgattatgcta
tgcactgggtgaggcagagtcatgcaaagagtctagagtggattggagtt
attagtacttactctggtgatgctatctacaaccagaagttcaagggcaa
ggccacaatgactgtcgacaaatcctccagcacagcctatctggaacttg
ccagactgacatctgacgattctgccatctattactgtgcaagaggggta
acttttgactactggggccaaggcaccactgtcacagtctcctcgg
(SEQ ID NO: 16)
D11 VH - Amino Acid
QIQLQQPGAELVRPGVSLKISCKGSGYTFTDYAMHWVRQSHAKSLEWIGV
ISTYSGDAIYNQKFKGKATMTVDKSSSTAYLELARLTSDDSAIYYCARGV
TFDYWGQGTTVTVSS (SEQ ID NO: 17)
D11 VH - CDR1-IMGT
ggctacacattcactgattatgct (SEQ ID NO: 18)
GYTFTDYA (SEQ ID NO: 19)
D11 VH - CDR2-IMGT
attagtacttactctggtgatgct (SEQ ID NO: 20)
ISTYSGDA (SEQ ID NO: 21)
D11 VH - CDR3-IMGT
gcaagaggggtaacttttgactac (SEQ ID NO: 22)
ARGVTFDY (SEQ ID NO: 23)

TABLE 1D
D11 VL Sequences

D11 VL-Nucleic Acid
gacatccagatgacacagactacatcctccctgtctgcctctctgggaga
cagagtcaccatcacttgcagggcaagtcaggacattagcaattatttag
aatggtatcagcagaaacagggaaaatctcctcagctcctggtctataat
gcaaaaaccttagcagaaggtgtgccatcaaggttcagtggcagtggatc
aggcacacagttttctctgaagatcaacagcctgcagcctgaagattttg
ggagttattactgtcaacatcattatgatactccgtacacgttcggaggg
gggaccaagctggaaataaaac (SEQ ID NO: 24)
D11 VL - Amino Acid
DIQMTQTTSSLSASLGDRVTITCRASQDISNYLEWYQQKQGKSPQLLVYN
AKTLAEGVPSRFSGSGSGTQFSLKINSLQPEDFGSYYCQHHYDTPYTFGG
GTKLEIK (SEQ ID NO: 25)
D11 VL - CDR1-IMGT
caggacattagcaattat (SEQ ID NO: 26)
QDISNY (SEQ ID NO: 27)
D11 VL - CDR2-IMGT
aatgcaaaa
NAK (SEQ ID NO: 28)
D11 VL - CDR3-IMGT
caacatcattatgatactccgtacacg (SEQ ID NO: 29)
QHHYDTPYT (SEQ ID NO: 30)

TABLE 1E
D16 VH Sequences

D16 VH - Nucleic Acid
caggtgcaaatgcagcagtctggagctgagctggtaaggcctgggacttc
agtgaagatatcctgcaaggcttctggctacaccttcactaactactggc
taggttgggtaaagcagaggcctggacatggacttgagtggattggagat
atttaccctggaggtggttatactaactacaatgagaagttcaagggcaa
ggccacactgactgcagacacatcctccagcactgcctacatgcagctca
gtagcctgacatctgaggactctgctgtctatttctgtgcaagagttact
ccggcttcctggggccaaggcaccactctcacagtctcctcgg (SEQ
ID NO: 31)
D16 VH - Amino Acid
QVQMQQSGAELVRPGTSVKISCKASGYTFTNYWLGWVKQRPGHGLEWIGD
IYPGGGYTNYNEKFKGKATLTADTSSSTAYMQLSSLTSEDSAVYFCARVT
PASWGQGTTLTVSS (SEQ ID NO: 32)
D16 VH - CDR1-IMGT
ggctacaccttcactaactactgg (SEQ ID NO: 3)
GYTFTNYW (SEQ ID NO: 4)
D16 VH - CDR2-IMGT
atttaccctggaggtggttatact (SEQ ID NO: 5)
IYPGGGYT (SEQ ID NO: 6)
D16 VH - CDR3-IMGT
gcaagagttactccggcttcc (SEQ ID NO: 7)
ARVTPAS (SEQ ID NO: 8)

TABLE 1F
D16 VL Sequences

D16 VL - Nucleic Acid
gatatccagatgacccagtctcaaaaattcatgtccacatcagtaggaga
cagggtcagcgtcacctgcaaggccagtcagaatgtgggtactaatgtag
cctggtatcaacagaaaccagggcaatctcctaaagcactgatttactcg
gcatcctaccggtacagtggagtccctgatcgcttcacaggcagtggatc
tgggacagatttcactctcaccatcagcaatgtgcagtctgaagacttgg
cagagtatttctgtcagcaatataacagctatccgtggacgttcggtgga
ggcaccaagctggaaatcaaac (SEQ ID NO: 33)
D16 VL - Amino Acid
DIQMTQSQKFMSTSVGDRVSVTCKASQNVGTNVAWYQQKPGQSPKALIYS
ASYRYSGVPDRFTGSGSGTDFTLTISNVQSEDLAEYFCQQYNSYPWTFGG
GTKLEIK (SEQ ID NO: 34)
D16 VL - CDR1-IMGT
cagaatgtgggtactaat (SEQ ID NO: 11)
QNVGTN (SEQ ID NO: 12)
D16 VL - CDR2-IMGT
tcggcatcc
SAS (SEQ ID NO: 13)
D16 VL - CDR3-IMGT
cagcaatataacagctatccgtggacg (SEQ ID NO: 35)
QQYNSYPWT (SEQ ID NO: 36)

TABLE 1G
D17 VH Sequences

D17 VH - Nucleic Acid Sequence
cagatccaactgcagcagtctggagctgagctggtaaggcctgggacttc
agtgaagatatcctgcaaggcttctggctacaccttcactaactactggc
taggttgggtaaagcagaggcctggacatggacttgagtggattggagat
atttaccctggaggtggttatactaactacaatgagaagttcaagggcaa
ggccacactgactgcagacacatcctccagcactgcctacatgcagctca
gtagcctgacatctgaggactctgctgtctatttctgtgcaagagttact
ccggcttcctggggccaaggcaccactctcacagtctctgcgg (SEQ
ID NO: 37)
D17 VH - Amino Acid Sequence
QIQLQQSGAELVRPGTSVKISCKASGYTFTNYWLGWVKQRPGHGLEWIGD
IYPGGGYTNYNEKFKGKATLTADTSSSTAYMQLSSLTSEDSAVYFCARVT
PASWGQGTTLTVSA (SEQ ID NO: 38)
D17 VH - CDR1-IMGT
ggctacaccttcactaactactgg (SEQ ID NO: 3)
GYTFTNYW (SEQ ID NO: 4)
D17 VH - CDR2-IMGT
atttaccctggaggtggttatact (SEQ ID NO: 5)
IYPGGGYT (SEQ ID NO: 6)
D17 VH - CDR3-IMGT
gcaagagttactccggcttcc (SEQ ID NO: 7)
ARVTPAS (SEQ ID NO: 8)

TABLE 1H
D17 VL Sequences

D17 VL - Nucleic Acid Sequence
gacattctgctgacccagtctcaaaaattcatgtccacatcagtaggaga
cagggtcagcgtcacctgcaaggccagtcagaatgtgggtactaatgtag
cctggtatcaacagaaaccagggcaatctcctaaagcactgatttactcg
gcatcctaccggtacagtggagtccctgatcgcttcacaggcagtggatc
tgggacagatttcactctcaccatcagcaatgtgcagtctgaagacttgg
cagagtatttctgtcagcaatataacagctatcctctcacgttcggaggg
gggaccaagctggaaataaaac (SEQ ID NO: 39)
D17 VL - Amino Acid Sequence
DILLTQSQKFMSTSVGDRVSVTCKASQNVGTNVAWYQQKPGQSPKALIYS
ASYRYSGVPDRFTGSGSGTDFTLTISNVQSEDLAEYFCQQYNSYPLTFGG
GTKLEIK (SEQ ID NO: 40)
D17 VL - CDR1-IMGT
cagaatgtgggtactaat (SEQ ID NO: 11)
QNVGTN (SEQ ID NO: 12)
D17 VL - CDR2-IMGT
tcggcatcc
SAS (SEQ ID NO: 13)
D17 VL - CDR3-IMGT
cagcaatataacagctatcctctcacg (SEQ ID NO: 41)
QQYNSYPLT (SEQ ID NO: 42)

TABLE 1I
D34 VH Sequences

D34 VH - Nucleic Acid Sequence
caggtccaactgcagcagtctggggctgagctggtgaggcctggggtctc
agtgaagatttcctgcaagggttctggctacacattcactgattatgcta
tgcactgggtgaagcagagtcatgcaaagagtctagagtggattggagtt
attagtacttactctggtgatgttagttacaaccagaagttcaagggcaa
ggccacaatgactgtcgacaaatcctccagcacagcctatatggaacttg
ccagactgacatctgaggattctgccatctattactgtgcaagaggggta
acttttgactcctggggccaaggcaccacggtcaccgtctcctcg (SEQ
ID NO: 43)
D34 VH - Amino Acid Sequence
QVQLQQSGAELVRPGVSVKISCKGSGYTFTDYAMHWVKQSHAKSLEWIGV
ISTYSGDVSYNQKFKGKATMTVDKSSSTAYMELARLTSEDSAIYYCARGV
TFDSWGQGTTVTVSS (SEQ ID NO: 44)
D34 VH - CDR1-IMGT
ggctacacattcactgattatgct (SEQ ID NO: 18)
GYTFTDYA (SEQ ID NO: 19)
D34 VH - CDR2-IMGT
attagtacttactctggtgatgtt (SEQ ID NO: 45)
ISTYSGDV (SEQ ID NO: 46)
D34 VH - CDR3-IMGT
gcaagaggggtaacttttgactcc (SEQ ID NO: 47)
ARGVTFDS (SEQ ID NO: 48)

TABLE 1J
D34 VL Sequences

D34 VL - Nucleic Acid Sequence
gacatccagatgacacagtctccagcctccctatctgcatctgtgggaga
aactgtcaccatcacatgtcgagcaagtgagaatagttacagttatttag
aatggtatcagcagaaacagggaaaatctcctcagctcctggtctataat
gcaaaaactttagcagaaggtgtgccatcaaggttcagtggcagtggatc
aggcacacagttttctctgaagatcaacagcctgcagcctgaagattttg
ggacttattactgtcaacatcattatggtactccgtacacgttcggaggg
gggaccaagctggaaataaaac (SEQ ID NO: 49)
D34 VL - Amino Acid Sequence
DIQMTQSPASLSASVGETVTITCRASENSYSYLEWYQQKQGKSPQLLVYN
AKTLAEGVPSRFSGSGSGTQFSLKINSLQPEDFGTYYCQHHYGTPYTFGG
GTKLEIK (SEQ ID NO: 50)
D34 VL - CDR1-IMGT
gagaatagttacagttat (SEQ ID NO: 51)
ENSYSY (SEQ ID NO: 52)
D34 VL - CDR2-IMGT
aatgcaaaa
NAK (SEQ ID NO: 28)
D34 VL - CDR3-IMGT
caacatcattatggtactccgtacacg (SEQ ID NO: 53)
QHHYGTPYT (SEQ ID NO: 54)

TABLE 1K
D36 VH Sequences

D36 VH - Nucleic Acid Sequence
caggtccaactgcagcagcctggggctgagctggtgaggcctggggtttc
actgaagatttcctgcaagggttctggctacacattcactgattatgcta
tgcactgggtgaggcagagtcatgcaaagagtctagagtggattggagtt
attagtacttactctggtgatgctctctacaaccagaagttcaagggcaa
ggccacaatgactgtcgacaaatcctccagcacagcctatctggaacttg
ccagactgacatctgaggattctgccatctattactgtgcgcgaggggta
acttttgactactggggccaaggcaccactctcacagtctcctcgg
(SEQ ID NO: 55)
D36 VH - Amino Acid Sequence
QVQLQQPGAELVRPGVSLKISCKGSGYTFTDYAMHWVRQSHAKSLEWIGV
ISTYSGDALYNQKFKGKATMTVDKSSSTAYLELARLTSEDSAIYYCARGV
TFDYWGQGTTLTVSS (SEQ ID NO: 56)
D36 VH - CDR1-IMGT
ggctacacattcactgattatgct (SEQ ID NO: 18)
GYTFTDYA (SEQ ID NO: 19)
D36 VH - CDR2-IMGT
attagtacttactctggtgatgct (SEQ ID NO: 20)
ISTYSGDA (SEQ ID NO: 21)
D36 VH - CDR3-IMGT
gcgcgaggggtaacttttgactac (SEQ ID NO: 57)
ARGVTFDY (SEQ ID NO: 23)

TABLE 1l
D36 VL Sequences

D36 VL - Nucleic Acid Sequence
gatgttgtgatgacccagtctccagcctccctatctgcatctgtgggaga
aactgtcaccatcacatgtcgagcaagtgagaatagttacagttatttag
aatggtatcagcagaaacagggaaaatctcctcagctcctggtctataat
gcaaaaactttagcagaaggtgtgccatcaaggttcagtggcagtggatc
aggcacacagttttctctgaagatcaacagcctgcagcctgaagattttg
ggacttattactgtcaacatcattatggtactccgtacacgttcggaggg
gggaccaagctggaaatgaaac (SEQ ID NO: 58)
D36 VL Amino Acid Sequence
DVVMTQSPASLSASVGETVTITCRASENSYSYLEWYQQKQGKSPQLLVYN
AKTLAEGVPSRFSGSGSGTQFSLKINSLQPEDFGTYYCQHHYGTPYTFGG
GTKLEMK (SEQ ID NO: 59)
D36 VL - CDR1-IMGT
gagaatagttacagttat (SEQ ID NO: 51)
ENSYSY (SEQ ID NO: 52)
D36 VL - CDR2-IMGT
aatgcaaaa
NAK (SEQ ID NO: 28)
D36 VL - CDR3-IMGT
caacatcattatggtactccgtacacg (SEQ ID NO: 53)
QHHYGTPYT (SEQ ID NO: 54)

TABLE 2A
Exemplary Clones - Heavy Chain Sequences

| | V-D-J- | | | | | | | JUNC- | J- | H- |
|---|---|---|---|---|---|---|---|---|---|---|
| ID | REGION | H-FR1 | CDRH1 | H-FR2 | CDRH2 | H-FR3 | CDRH3 | TION | REGION | FR4 |
| AHH | DVKLVESGGGL | DVKL | GFTFS | MSWV | ISSGG | YYPDS | ARGEI | CARGE | WFAYW | WGQG |
| 03702 | VKPGGSLKLSC | VESG | SYA | RQTP | ST | VKGRF | WGKAW | IWGKA | GQGTL | TLVT |
| | AASGFTFSSYA | GGLV | (SEQ | EKRL | (SEQ | TISRD | FAY | WFAYW | VTVSA | VSA |
| | MSWVRQTPEKR | KPGG | ID | EWVA | ID | NARNI | (SEQ | (SEQ | (SEQ | (SEQ |
| | LEWVASISSGG | SLKL | NO: | S | NO: | LYLQM | ID | ID | ID | ID |
| | STYYPDSVKGR | SCAA | 151) | (SEQ | 224) | SSLRS | NO: | NO: | NO: | NO: |
| | FTISRDNARNI | S | | ID | | EDTAM | 297) | 345) | 392) | 421) |
| | LYLQMSSLRSE | (SEQ | | NO: | | YYC | | | | |
| | DTAMYYCARGE | ID | | 184) | | (SEQ | | | | |
| | IWGKAWFAYWG | NO: | | | | ID | | | | |
| | QGTLVTVSA | 108) | | | | NO: | | | | |
| | (SEQ ID NO: | | | | | 259) | | | | |
| | 60) | | | | | | | | | |
| AHH | QVQMKQSGPEL | QVQM | GYTLT | MHWV | INTET | TYADD | AWGNH | CAWGN | YWGQG | WGQG |
| 03703 | KKPGETVKISC | KQSG | DYS | KQAP | GEP | FKGRF | Y | HYW | TTVTV | TTVT |
| | KASGYTLTDYS | PELK | (SEQ | GKGL | (SEQ | AFSLE | (SEQ | (SEQ | SS | VSS |
| | MHWVKQAPGKG | KPGE | ID | KWMG | ID | TSAST | ID | ID | (SEQ | (SEQ |
| | LKWMGWINTET | TVKI | NO: | W | NO: | AYLQI | NO: | NO: | ID | ID |
| | GEPTYADDFKG | SCKA | 152) | (SEQ | 225) | NNLKN | 298) | 346) | NO: | NO: |
| | RFAFSLETSAS | S | | ID | | EDTAT | | | 393) | 422) |
| | TAYLQINNLKN | (SEQ | | NO: | | YFC | | | | |
| | EDTATYFCAWG | ID | | 185) | | (SEQ | | | | |
| | NHYWGQGTTVT | NO: | | | | ID | | | | |
| | VSS (SEQ ID | 109) | | | | NO: | | | | |
| | NO: 61) | | | | | 260) | | | | |
| AHH | QIQLQQSGPEL | QIQL | GYTFT | MHWV | INPYN | KYNEK | ARDGY | CARDG | AYWGQ | WGQG |
| 03704 | VKPGASVKMSC | QQSG | SYV | KQKP | DGT | FKGKA | YVGPA | YYVGP | GTLVT | TLVT |
| | KASGYTFTSYV | PELV | (SEQ | GQGL | (SEQ | TLTSD | Y | AYW | VSA | VSA |
| | MHWVKQKPGQG | KPGA | ID | EWIG | ID | KSSST | (SEQ | (SEQ | (SEQ | (SEQ |
| | LEWIGYINPYN | SVKM | NO: | Y | NO: | AYMEL | ID | ID | ID | ID |
| | DGTKYNEKFKG | SCKA | 153) | (SEQ | 226) | SSLTS | NO: | NO: | NO: | NO: |
| | KATLTSDKSSS | S | | ID | | EDSAV | 299) | 347) | 394) | 421) |
| | TAYMELSSLTS | (SEQ | | NO: | | YYC | | | | |
| | EDSAVYYCARD | ID | | 186) | | (SEQ | | | | |
| | GYYVGPAYWGQ | NO: | | | | ID | | | | |
| | GTLVTVSA | 110) | | | | NO: | | | | |
| | (SEQ ID NO: | | | | | 261) | | | | |
| | 62) | | | | | | | | | |
| AHH | EVLLVESGGGL | EVLL | GFAFS | MSWV | ISSGG | YYPDT | ARNYR | CARNY | WFAYW | WGQG |
| 03705 | VKPGGSLKLSC | VESG | SYD | RQTP | GST | VKGRF | SWFAY | RSWFA | GQGTL | TLVT |
| | AASGFAFSSYD | GGLV | (SEQ | EKRL | (SEQ | TISRD | (SEQ | YW | VTVSA | VSA |
| | MSWVRQTPEKR | KPGG | ID | EWVA | ID | NAKNT | ID | (SEQ | (SEQ | (SEQ |
| | LEWVAYISSGG | SLKL | NO: | Y | NO: | LYLQM | NO: | ID | ID | ID |
| | GSTYYPDTVKG | SCAA | 154) | (SEQ | 227) | SSLKS | 300) | NO: | NO: | NO: |
| | RFTISRDNAKN | S | | ID | | EDTAM | | 348) | 392) | 421) |
| | TLYLQMSSLKS | (SEQ | | NO: | | YYC | | | | |
| | EDTAMYYCARN | ID | | 187) | | (SEQ | | | | |
| | YRSWFAYWGQG | NO: | | | | ID | | | | |
| | TLVTVSA | 111) | | | | NO: | | | | |
| | (SEQ ID NO: | | | | | 262) | | | | |
| | 63) | | | | | | | | | |
| AHH | QVQLQQSGAEL | QVQL | GYAFT | IEWV | INPGS | NYNEK | ARKGS | CARKG | AYWGQ | WGQG |
| 03706 | VRPGTSVKVSC | QQSG | NYL | KQRP | GGT | FKGKA | LTGVL | SLTGV | GTLVT | TLVT |
| | KASGYAFTNYL | AELV | (SEQ | GQGL | (SEQ | TLTAD | AY | LAYW | VSA | VSA |
| | IEWVKQRPGQG | RPGT | ID | EWIG | ID | KSSST | (SEQ | (SEQ | (SEQ | (SEQ |
| | LEWIGVINPGS | SVKV | NO: | V | NO: | AYMQL | ID | ID | ID | ID |
| | GGTNYNEKFKG | SCKA | 155) | (SEQ | 228) | SSLTS | NO: | NO: | NO: | NO: |
| | KATLTADKSSS | S | | ID | | DDSAV | 301) | 349) | 394) | 421) |
| | TAYMQLSSLTS | (SEQ | | NO: | | YFC | | | | |
| | DDSAVYFCARK | ID | | 188) | | (SEQ | | | | |
| | GSLTGVLAYWG | NO: | | | | ID | | | | |
| | QGTLVTVSA | 112) | | | | NO: | | | | |
| | (SEQ ID NO: | | | | | 263) | | | | |
| | 64) | | | | | | | | | |
| AHH | QVQLQQSGAEL | QVQL | GYIFT | MNWV | IDPSD | HYNQM | ARRGL | CARRG | WFAYW | WGQG |
| 03707 | VRPGASVKLSC | QQSG | NYW | KQRP | SET | SKDKA | RAWFA | LRAWF | GQGTL | TLVT |
| | KASGYIFTNYW | AELV | (SEQ | GQGL | (SEQ | TLTVD | Y | AYW | VTVSA | VSA |
| | MNWVKQRPGQG | RPGA | ID | EWIG | ID | KSSST | (SEQ | (SEQ | (SEQ | (SEQ |
| | LEWIGMIDPSD | SVKL | NO: | M | NO: | AYMQL | ID | ID | ID | ID |
| | SETHYNQMSKD | SCKA | 156) | (SEQ | 229) | SSLTS | NO: | NO: | NO: | NO: |
| | KATLTVDKSSS | S | | ID | | EDSAV | 302) | 350) | 392) | 421) |
| | TAYMQLSSLTS | (SEQ | | NO: | | YYC | | | | |
| | EDSAVYYCARR | ID | | 189) | | (SEQ | | | | |
| | GLRAWFAYWGQ | NO: | | | | ID | | | | |
| | GTLVTVSA | 113) | | | | NO: | | | | |
| | (SEQ ID NO: | | | | | 264) | | | | |
| | 65) | | | | | | | | | |
| AHH | QVQLQQPGAEL | QVQL | GYTFT | MHWV | INPST | EYNQK | ARLDY | CARLD | FAYWG | WGQG |
| 03708 | AKPGASVKMSC | QQPG | SYW | KQRP | GYT | FKDKA | YGSSR | YYGSS | QGTLV | TLVT |
| | KASGYTFTSYW | AELA | (SEQ | GQGL | (SEQ | TLTAD | GFAY | RGFAY | TVSA | VSA |
| | MHWVKQRPGQG | KPGA | ID | EWIG | ID | KSSST | (SEQ | W | (SEQ | (SEQ |
| | LEWIGYINPST | SVKM | NO: | Y | NO: | AYMQL | ID | (SEQ | ID | ID |
| | GYTEYNQKFKD | SCKA | 157) | (SEQ | 230) | SSLTS | NO: | ID | NO: | NO: |
| | KATLTADKSSS | S | | ID | | EDSAV | 303) | NO: | 395) | 421) |
| | TAYMQLSSLTS | (SEQ | | NO: | | YYC | | 351) | | |
| | EDSAVYYCARL | ID | | 190) | | (SEQ | | | | |
| | DYYGSSRGFAY | NO: | | | | ID | | | | |
| | WGQGTLVTVSA | 114) | | | | NO: | | | | |
| | (SEQ ID NO: | | | | | 265) | | | | |
| | 66) | | | | | | | | | |
| AHH | QVQLQQSGAEL | QVQL | GYTFT | MYWV | INPSN | NFNEK | TNGGG | CTNGG | GGWYW | WGQG |
| 03709 | VKPGASVKLSC | QQSG | SYY | KQRP | GGT | FKSKA | WY | GWYW | GQGTT | TTVT |
| | KASGYTFTSYY | AELV | (SEQ | GQGL | (SEQ | TLTVD | (SEQ | (SEQ | VTVSS | VSS |
| | MYWVKQRPGQG | KPGA | ID | EWIG | ID | KSSST | ID | ID | (SEQ | (SEQ |
| | LEWIGEINPSN | SVKL | NO: | E | NO: | AYMQL | NO: | NO: | ID | ID |
| | GGTNFNEKFKS | SCKA | 158) | (SEQ | 231) | SSLTS | 304) | 352) | NO: | NO: |
| | KATLTVDKSSS | S | | ID | | EDSAV | | | 396) | 422) |
| | TAYMQLSSLTS | (SEQ | | NO: | | YYC | | | | |
| | EDSAVYYCTNG | ID | | 191) | | (SEQ | | | | |
| | GGWYWGQGTTV | NO: | | | | ID | | | | |
| | TVSS (SEQ | 115) | | | | NO: | | | | |
| | ID NO: 67) | | | | | 266) | | | | |
| AHH | QIQLVQSGPEL | QIQL | GYTFT | MNWV | INTYT | TYADD | ASYYD | CASYY | FAYWG | WGQG |
| 03711 | KKPGETVKISC | VQSG | NYG | KQTP | GEP | FKGRF | STYVG | DSTYV | QGTLV | TLVT |
| | KASGYTFTNYG | PELK | (SEQ | GKGL | (SEQ | AFSLE | FAY | GFAYW | TVSA | VSA |
| | MNWVKQTPGKG | KPGE | ID | KWMG | ID | TSAST | (SEQ | (SEQ | (SEQ | (SEQ |
| | LKWMGWINTYT | TVKI | NO: | W | NO: | AYLQI | ID | ID | ID | ID |
| | GEPTYADDFKG | SCKA | 159) | (SEQ | 232) | NNLKN | NO: | NO: | NO: | NO: |
| | RFAFSLETSAS | S | | ID | | EDTAT | 305) | 353) | 395) | 421) |
| | TAYLQINNLKN | (SEQ | | NO: | | YFC | | | | |
| | EDTATYFCASY | ID | | 192) | | (SEQ | | | | |
| | YDSTYVGFAYW | NO: | | | | ID | | | | |
| | GQGTLVTVSA | 116) | | | | NO: | | | | |
| | (SEQ ID NO: | | | | | 260) | | | | |
| | 68) | | | | | | | | | |
| AHH | EVLLVESGGGL | EVLL | GFTFS | MYWV | ISNGG | YYPDT | ASPLG | CASPL | FAYWG | WGQG |
| 03712 | VQPGGSLKLSC | VESG | DYY | RQTP | GST | VKGRF | YDGFA | GYDGF | QGTLV | TLVT |
| | ATSGFTFSDYY | GGLV | (SEQ | EKRL | (SEQ | TISRD | Y | AYW | TVSA | VSA |
| | MYWVRQTPEKR | QPGG | ID | EWVA | ID | NAKNT | (SEQ | (SEQ | (SEQ | (SEQ |
| | LEWVAYISNGG | SLKL | NO: | Y | NO: | LYLQM | ID | ID | ID | ID |
| | GSTYYPDTVKG | SCAT | 160) | (SEQ | 233) | SRLKS | NO: | NO: | NO: | NO: |
| | RFTISRDNAKN | S | | ID | | EDTAM | 306) | 354) | 395) | 421) |
| | TLYLQMSRLKS | (SEQ | | NO: | | YYC | | | | |
| | EDTAMYYCASP | ID | | 193) | | (SEQ | | | | |
| | LGYDGFAYWGQ | NO: | | | | ID | | | | |
| | GTLVTVSA | 117) | | | | NO: | | | | |
| | (SEQ ID NO: | | | | | 267) | | | | |
| | 69) | | | | | | | | | |
| AHH | QVQMKQSGPGL | QVQM | GFSLT | VHWV | IWAGG | NYNSA | ARGA | CARGA | YFDYW | WGQG |
| 03713 | VAPSQSLSITC | KQSG | SYG | RQPP | ST | LMSRL | YFDY | YFDYW | GQGTT | TTVT |
| | TVSGFSLTSYG | PGLV | (SEQ | GKGL | (SEQ | SISKD | (SEQ | (SEQ | VTVSS | VSS |
| | VHWVRQPPGKG | APSQ | ID | EWLG | ID | NSKSQ | ID | ID | (SEQ | (SEQ |
| | LEWLGVIWAGG | SLSI | NO: | V | NO: | VFLKM | NO: | NO: | ID | ID |
| | STNYNSALMSR | TCTV | 161) | (SEQ | 234) | NSLQT | 307) | 355) | NO: | NO: |
| | LSISKDNSKSQ | S | | ID | | DDTAM | | | 397) | 422) |
| | VFLKMNSLQTD | (SEQ | | NO: | | YYC | | | | |
| | DTAMYYCARGA | ID | | 194) | | (SEQ | | | | |
| | YFDYWGQGTTV | NO: | | | | ID | | | | |
| | TVSS (SEQ | 118) | | | | NO: | | | | |
| | ID NO: 70) | | | | | 268) | | | | |
| AHH | QVQLQQSGAEL | QVQL | GYTFT | MNWV | IDPYD | HYNQK | ARSP | CARSP | WFAYW | WGQG |
| 03714 | VRPGASVKLSC | QQSG | SYW | KQRP | SET | FKDKA | AYYG | AYYGN | GQGTL | TLVT |
| | KASGYTFTSYW | AELV | (SEQ | EQGL | (SEQ | ILTVD | NLWF | LWFAY | VTVSA | VSA |
| | MNWVKQRPEQG | RPGA | ID | EWIG | ID | KSSST | AY | W | (SEQ | (SEQ |
| | LEWIGRIDPYD | SVKL | NO: | R | NO: | AYMQL | (SEQ | (SEQ | ID | ID |
| | SETHYNQKFKD | SCKA | 157) | (SEQ | 235) | SSLTS | ID | ID | NO: | NO: |
| | KAILTVDKSSS | S(SEQ | | ID | | EDSAV | NO: | NO: | 392) | 421) |
| | TAYMQLSSLTS | ID NO: | | NO: | | YYC | 308) | 356) | | |
| | EDSAVYYCARS | 113) | | 195) | | (SEQ | | | | |
| | PAYYGNLWFAY | | | | | ID | | | | |
| | WGQGTLVTVSA | | | | | NO: | | | | |
| | (SEQ ID NO: | | | | | 269) | | | | |
| | 71) | | | | | | | | | |
| AHH | QVQLQQSGAEL | QVQL | GYTFT | IHWV | INPSS | NYNQK | ARWD | CARW | WGQGT | WGQG |
| 03715 | ARPGASVKMSC | QQSG | SYT | KQRP | GYT | FKDKA | GAY | DGAY | PVTVS | TPVT |
| | KASGYTFTSYT | AELA | (SEQ | GQGL | (SEQ | TLTAD | (SEQ | W | S | VSS |
| | IHWVKQRPGQG | RPGA | ID | EWIG | ID | KSSST | ID | (SEQ | (SEQ | (SEQ |
| | LEWIGYINPSS | SVKM | NO: | (SEQ | NO: | AYMQL | NO: | ID | ID | ID |
| | GYTNYNQKFKD | SCKA | 162) | ID | 236) | SSLTS | 309) | NO: | NO: | NO: |
| | KATLTADKSSS | S | | NO: | | EDSAV | | 357) | 398) | 398) |
| | TAYMQLSSLTS | (SEQ | | 196) | | YYC | | | | |
| | EDSAVYYCARW | ID | | | | (SEQ | | | | |
| | DGAYWGQGTPV | NO: | | | | ID | | | | |
| | TVSS (SEQ | 119) | | | | NO: | | | | |
| | ID NO: 72) | | | | | 270) | | | | |
| AHH | DVKLVESGGGL | DVKL | GFTFS | MSWV | ISSGG | YYPDT | ARGG | CARGG | FAYWG | WGQG |
| 03716 | VKPGGSLKLSC | VESG | SYA | RQFP | SYT | VTGRF | MITP | MITPF | QGTLV | TLVT |
| | AASGFTFSSYA | GGLV | (SEQ | EKRL | (SEQ | TISRD | FAY | AYW | TVSS | VSS |
| | MSWVRQFPEKR | KPGG | ID | EWVA | ID | NAKNT | (SEQ | (SEQ | (SEQ | (SEQ |
| | LEWVAEISSGG | SLKL | NO: | E | NO: | LYLEM | ID | ID | ID | ID |
| | SYTYYPDTVTG | SCAA | 151) | (SEQ | 237) | SSLRS | NO: | NO: | NO: | NO: |
| | RFTISRDNAKN | S | | ID | | EDTAM | 310) | 358) | 399) | 413) |
| | TLYLEMSSLRS | (SEQ | | NO: | | YYC | | | | |
| | EDTAMYYCARG | ID | | 197) | | (SEQ | | | | |
| | GMITPFAYWGQ | NO: | | | | ID | | | | |
| | GTLVTVSS | 108) | | | | NO: | | | | |
| | (SEQ ID NO: | | | | | 271) | | | | |
| | 73) | | | | | | | | | |
| AHH | QVILKESGPGI | QVIL | GFSLS | VGWI | IWWND | YYNPA | ARIG | CARIG | YWYFD | WGAG |
| 03717 | LQPSQTLSLTC | KESG | TSGMS | RQPS | DK | LKSRL | GNDG | GNDGY | VWGAG | TTVT |
| | SFSGFSLSTSG | PGIL | (SEQ | GKGL | (SEQ | TISKD | YYWY | YWYFD | TTVTV | VSS |
| | MSVGWIRQPSG | QPSQ | ID | EWLA | ID | TSNNQ | FDV | VW | SS | (SEQ |
| | KGLEWLAHIWW | TLSL | NO: | H | NO: | VFLKI | (SEQ | (SEQ | (SEQ | ID |
| | NDDKYYNPALK | TCSF | 163) | (SEQ | 238) | ASVVT | ID | ID | ID | NO: |
| | SRLTISKDTSN | S | | ID | | ADTAT | NO: | NO: | NO: | 423) |
| | NQVFLKIASVV | (SEQ | | NO: | | YYC | 311) | 359) | 400) | |
| | TADTATYYCAR | ID | | 198) | | (SEQ | | | | |
| | IGGNDGYYWYF | NO: | | | | ID | | | | |
| | DVWGAGTTVTV | 120) | | | | NO: | | | | |
| | SS (SEQ ID | | | | | 272) | | | | |
| | NO: 74) | | | | | | | | | |
| AHH | EVKIEESGGGL | EVKI | GFTFS | MDWV | IRSKA | YYAES | TPQF | CTPQF | FAYWG | WGQG |
| 03718 | VQPGGSMKLSC | EESG | DAW | RQSP | NNHAT | VKGRF | AY | AYW | QGTTV | TTVT |
| | AASGFTFSDAW | GGLV | (SEQ | EKGL | (SEQ | TISRD | (SEQ | (SEQ | TVSS | VSS |
| | MDWVRQSPEKG | QPGG | ID | EWVA | ID | DSKSS | ID | ID | (SEQ | (SEQ |
| | LEWVAEIRSKA | SMKL | NO: | E | NO: | VYLQM | NO: | NO: | ID | ID |
| | NNHATYYAESV | SCAA | 164) | (SEQ | 239) | NSLRA | 312) | 360) | NO: | NO: |
| | KGRFTISRDDS | S | | ID | | EDTG | | | 401) | 422) |
| | KSSVYLQMNSL | (SEQ | | NO: | | IYYC | | | | |
| | RAEDTGIYYCT | ID | | 199) | | (SEQ | | | | |
| | PQFAYWGQGTT | NO: | | | | ID | | | | |
| | VTVSS (SEQ | 121) | | | | NO: | | | | |
| | ID NO: 75) | | | | | 273) | | | | |
| AHH | EVMLVESGGGL | EVML | GFTFS | MSWV | INSNG | YYPDS | ASHY | CASHY | YWGQG | WGQG |
| 03719 | VQPGGSLKLSC | VESG | SYG | RQTP | GST | VKGRF | DEGY | DEGYW | TSLTV | TSLT |
| | AASGFTFSSYG | GGLV | (SEQ | DKRL | (SEQ | TISRD | (SEQ | (SEQ | SS | VSS |
| | MSWVRQTPDKR | QPGG | ID | ELVA | ID | NAKNT | ID | ID | (SEQ | (SEQ |
| | LELVATINSNG | SLKL | NO: | T | NO: | LYLQM | NO: | NO: | ID | ID |
| | GSTYYPDSVKG | SCAA | 165) | (SEQ | 240) | SSLKS | 313) | 361) | NO: | NO: |
| | RFTISRDNAKN | S | | ID | | EDTAM | | | 402) | 424) |
| | TLYLQMSSLKS | (SEQ | | NO: | | YYC | | | | |
| | EDTAMYYCASH | ID | | 200) | | (SEQ | | | | |
| | YDEGYWGQGTS | NO: | | | | ID | | | | |
| | LTVSS (SEQ | 122) | | | | NO: | | | | |
| | ID NO: 76) | | | | | 274) | | | | |
| AHH | EVKLEESGGGL | EVKL | GFTFS | MNWV | IRLKS | HYAES | TGSDY | CTGSD | DYWGQ | WGQG |
| 03720 | VQPGGSMKLSC | EESG | NYW | RQSP | NNYAT | VKGRF | (SEQ | YW | GTTVT | TTVT |
| | VASGFTFSNYW | GGLV | (SEQ | EKGL | (SEQ | TISRD | ID | (SEQ | VSS | VSS |
| | MNWVRQSPEKG | QPGG | ID | EWVA | ID | DSKSS | NO: | ID | (SEQ | (SEQ |
| | LEWVAEIRLKS | SMKL | NO: | E | NO: | VYLQM | 314) | NO: | ID | ID |
| | NNYATHYAESV | SCVA | 166) | (SEQ | 241) | NNLRA | | 362) | NO: | NO: |
| | KGRFTISRDDS | S | | ID | | EDTGI | | | 403) | 422) |
| | KSSVYLQMNNL | (SEQ | | NO: | | YYC | | | | |
| | RAEDTGIYYCT | ID | | 201) | | (SEQ | | | | |
| | GSDYWGQGTTV | NO: | | | | ID | | | | |
| | TVSS (SEQ | 123) | | | | NO: | | | | |
| | ID NO: 77) | | | | | 275) | | | | |
| AHH | QVQLQQSGPEL | QVQL | GDTFS | IHWV | FNPYS | KYNEK | GSGYD | CGSGY | WFACW | WGQG |
| 03721 | VKPGASVKMSC | QQSG | SYV | KQKP | DDI | FKGKA | GYYDW | DGYYD | GQGTL | TLVT |
| | KASGDTFSSYV | PELV | (SEQ | GQGL | (SEQ | TLTSD | FAC | WFACW | VTVSA | VSA |
| | IHWVKQKPGQG | KPGA | ID | EWIG | ID | KSSST | (SEQ | (SEQ | (SEQ | (SEQ |
| | LEWIGYFNPYS | SVKM | NO: | (SEQ | NO: | AYMEL | ID | ID | ID | ID |
| | DDIKYNEKFKG | SCKA | 167) | ID | 242) | SSLTS | NO: | NO: | NO: | NO: |
| | KATLTSDKSSS | S | | NO: | | EDSAV | 315) | 363) | 404) | 421) |
| | TAYMELSSLTS | (SEQ | | 202) | | YYC | | | | |
| | EDSAVYYCGSG | ID | | | | (SEQ | | | | |
| | YDGYYDWFACW | NO: | | | | ID | | | | |
| | GQGTLVTVSA | 124) | | | | NO: | | | | |
| | (SEQ ID NO: | | | | | 261) | | | | |
| | 78) | | | | | | | | | |
| AHH | QIQLAQSGPEL | QIQL | GYSFT | MNWV | INTYT | TYADD | AREPK | CAREP | DYWGQ | WGQG |
| 03722 | KKPGETVKISC | AQSG | KNG | KQAP | GEP | FKGRF | TLDY | KTLDY | GTTVT | TTVT |
| | KASGYSFTKNG | PELK | (SEQ | GKGL | (SEQ | AFSLE | (SEQ | W | VSS | VSS |
| | MNWVKQAPGKG | KPGE | ID | KWMG | ID | TSAST | ID | (SEQ | (SEQ | (SEQ |
| | LKWMGWINTYT | TVKI | NO: | W | NO: | AYLQI | NO: | ID | ID | ID |
| | GEPTYADDFKG | SCKA | 168) | (SEQ | 232) | NNLKN | 316) | NO: | NO: | NO: |
| | RFAFSLETSAS | S | | ID | | EDTAT | | 364) | 403) | 422) |
| | TAYLQINNLKN | (SEQ | | NO: | | YFC | | | | |
| | EDTATYFCARE | ID | | 203) | | (SEQ | | | | |
| | PKTLDYWGQGT | NO: | | | | ID | | | | |
| | TVTVSS (SEQ | 125) | | | | NO: | | | | |
| | ID NO: 79) | | | | | 260) | | | | |
| AHH | QVQLQQSGAEL | QVQL | GYTFT | MNWV | IDPSD | HYNQK | ANWA | CANWA | WFAYW | WGQG |
| 03724 | VKPGAPVKLSC | QQSG | SYW | KQRP | SET | FKDKA | WFAY | WFAYW | GQGTL | TLVT |
| | KASGYTFTSYW | AELV | (SEQ | GRGL | (SEQ | TLTVD | (SEQ | (SEQ | VTVSA | VSA |
| | MNWVKQRPGRG | KPGA | ID | EWIG | ID | KSSST | ID | ID | (SEQ | (SEQ |
| | LEWIGRIDPSD | PVKL | NO: | R | NO: | AYIQL | NO: | NO: | ID | ID |
| | SETHYNQKFKD | SCKA | 157) | (SEQ | 229) | SSLTS | 317) | 365) | NO: | NO: |
| | KATLTVDKSSS | S | | ID | | EDSAV | | | 392) | 421) |
| | TAYIQLSSLTS | (SEQ | | NO: | | YYC | | | | |
| | EDSAVYYCANW | ID | | 204) | | (SEQ | | | | |
| | AWFAYWGQGTL | NO: | | | | ID | | | | |
| | VTVSA (SEQ | 126) | | | | NO: | | | | |
| | ID NO: 80) | | | | | 276) | | | | |
| AHH | QMQLKESGTEL | QMQL | GYTFN | MNWV | IDPYD | HYNQK | ARPY | CARPY | FAYWG | WGQG |
| 03726 | VRPGASVKLSC | KESG | SHW | KQRP | SET | FKDKA | DYDG | DYDGF | QGTLV | TLVT |
| | KASGYTFNSHW | TELV | (SEQ | EQGL | (SEQ | ILTVD | FAY | AYW | TVSA | VSA |
| | MNWVKQRPEQG | RPGA | ID | EWIG | ID | KSSST | (SEQ | (SEQ | (SEQ | (SEQ |
| | LEWIGKIDPYD | SVKL | NO: | K | NO: | AYMQL | ID | ID | ID | ID |
| | SETHYNQKFKD | SCKA | 169) | (SEQ | 235) | SSLTS | NO: | NO: | NO: | NO: |
| | KAILTVDKSSS | S | | ID | | EDSAV | 318) | 366) | 395) | 421) |
| | TAYMQLSSLTS | (SEQ | | NO: | | YYC | | | | |
| | EDSAVYYCARP | ID | | 205) | | (SEQ | | | | |
| | YDYDGFAYWGQ | NO: | | | | ID | | | | |
| | GTLVTVSA | 127) | | | | NO: | | | | |
| | (SEQ ID NO: | | | | | 269) | | | | |
| | 81) | | | | | | | | | |
| AHH | QIQLQQSGAEL | QIQL | GYTF | INWV | INPYN | SYNQK | ADGD | CADGD | YFDYW | WGQG |
| 03727 | VRPGASVKISC | QQSG | TNHH | KQRP | DYT | FKGKA | YYFD | YYFDY | GQGTS | TSLT |
| | KAFGYTFTNHH | AELV | (SEQ | GQGL | (SEQ | TLTVD | Y | W | LTVSS | VSS |
| | INWVKQRPGQG | RPGA | ID | DWIGY | ID | KSSST | (SEQ | (SEQ | (SEQ | (SEQ |
| | LDWIGYINPYN | SVKI | NO: | (SEQ | NO: | AYMEL | ID | ID | ID | ID |
| | DYTSYNQKFKG | SCKA | 170) | ID | 243) | SSLTS | NO: | NO: | NO: | NO: |
| | KATLTVDKSSS | F | | NO: | | EDSAV | 319) | 367) | 405) | 424) |
| | TAYMELSSLTS | (SEQ | | 206) | | YYC | | | | |
| | EDSAVYYCADG | ID | | | | (SEQ | | | | |
| | DYYFDYWGQGT | NO: | | | | ID | | | | |
| | SLTVSS (SEQ | 128) | | | | NO: | | | | |
| | ID NO: 82) | | | | | 277) | | | | |
| AHH | DVLLVESGGDL | DVLL | GLTF | MSWV | ISSGG | YYVDS | ARQD | CARQD | FDYWG | WGQG |
| 03728 | VKPGGSLKLSC | VESG | SSYG | RQTP | SYI | VKGRF | DGYY | DGYYR | QGTTL | TTLT |
| | VVSGLTFSSYG | GDLV | (SEQ | DKRL | (SEQ | TISRD | RIFD | IFDYW | TVSS | VSS |
| | MSWVRQTPDKR | KPGG | ID | EWVA | ID | NAKNT | Y | (SEQ | (SEQ | (SEQ |
| | LEWVATISSGG | SLKL | NO: | T | NO: | LYLQM | (SEQ | ID | ID | ID |
| | SYIYYVDSVKG | SCVVS | 171) | (SEQ | 244) | SSLKS | ID | NO: | NO: | NO: |
| | RFTISRDNAKN | (SEQ | | ID | | EDTAI | NO: | 368) | 406) | 425) |
| | TLYLQMSSLKS | ID | | NO: | | YYC | 320) | | | |
| | EDTAIYYCARQ | NO: | | 207) | | (SEQ | | | | |
| | DDGYYRIFDYW | 129) | | | | ID | | | | |
| | GQGTTLTVSS | | | | | NO: | | | | |
| | (SEQ ID NO: | | | | | 278) | | | | |
| | 83) | | | | | | | | | |
| AHH | EVKIEESGGGL | EVKI | GFTF | MDWV | IRSKA | YYAES | TNYG | CTNYG | DYWGQ | WGQG |
| 03729 | VQPGGSMKLSC | EESG | NDAW | RQSP | NNHAT | VKGRF | SNPL | SNPLD | GTTLT | TTLT |
| | AASGFTFNDAW | GGLV | (SEQ | EKGL | (SEQ | TISRD | DY | YW | IPS | IPS |
| | MDWVRQSPEKG | QPGG | ID | EWVA | ID | DSQSS | (SEQ | (SEQ | (SEQ | (SEQ |
| | LEWVAEIRSKA | SMKL | NO: | E | NO: | VYLQM | ID | ID | ID | ID |
| | NNHATYYAESV | SCAA | 172) | (SEQ | 239) | NSLRT | NO: | NO: | NO: | NO: |
| | KGRFTISRDDS | S | | ID | | ENTGI | 321) | 369) | 407) | 426) |
| | QSSVYLQMNSL | (SEQ | | NO: | | YYC | | | | |
| | RTENTGIYYCT | ID | | 199) | | (SEQ | | | | |
| | NYGSNPLDYWG | NO: | | | | ID | | | | |
| | QGTTLTIPS | 121) | | | | NO: | | | | |
| | (SEQ ID NO: | | | | | 279) | | | | |
| | 84) | | | | | | | | | |
| AHH | QVTLKESGPGL | QVTL | GFSL | VHWV | IWAGG | NYNSA | AREG | CAREG | WFAYW | WGQG |
| 03730 | VAPSQSLSITC | KESG | TSYG | RQPP | ST | LMSRL | TGPW | TGPWF | GQGTT | TTAP |
| | TVSGFSLTSYG | PGLV | (SEQ | GKGL | (SEQ | GISKD | FAY | AYW | APSP | SP |
| | VHWVRQPPGKG | APSQ | ID | EWLG | ID | NSKSQ | (SEQ | (SEQ | (SEQ | (SEQ |
| | LEWLGVIWAGG | SLSI | NO: | V | NO: | VFLKM | ID | ID | ID | ID |
| | STNYNSALMSR | TCTV | 161) | (SEQ | 234) | NSLQT | NO: | NO: | NO: | NO: |
| | LGISKDNSKSQ | S | | ID | | DESAF | 322) | 370) | 408) | 427) |
| | VFLKMNSLQTD | (SEQ | | NO: | | YYC | | | | |
| | ESAFYYCAREG | ID | | 194) | | (SEQ | | | | |
| | TGPWFAYWGQG | NO: | | | | ID | | | | |
| | TTAPSP (SEQ | 130) | | | | NO: | | | | |
| | ID NO: 85) | | | | | 280) | | | | |
| AHH | DVQLQQSGPDL | DVQL | GYSI | WHWI | IHYSG | NYNPS | ARDP | CARDP | FAYWG | WGQG |
| 03731 | VKPSQSLSLTC | QQSG | TSGY | RQFP | ST | LKSRI | PFAY | PFAYW | QGTLV | TLVT |
| | TVTGYSITSGY | PDLV | S | GNKL | (SEQ | SITRD | (SEQ | (SEQ | TVSA | VSA |
| | SWHWIRQFPGN | KPSQ | (SEQ | EWMG | ID | TSKNQ | ID | ID | (SEQ | (SEQ |
| | KLEWMGYIHYS | SLSL | ID | Y | NO: | FFLQL | NO: | NO: | ID | ID |
| | GSTNYNPSLKS | TCTV | NO: | (SEQ | 245) | NSVTT | 323) | 371) | NO: | NO: |
| | RISITRDTSKN | T | 173) | ID | | EGTAT | | | 395) | 421) |
| | QFFLQLNSVTT | (SEQ | | NO: | | YYC | | | | |
| | EGTATYYCARD | ID | | 208) | | (SEQ | | | | |
| | PPFAYWGQGTL | NO: | | | | ID | | | | |
| | VTVSA (SEQ | 131) | | | | NO: | | | | |
| | ID NO: 86) | | | | | 281) | | | | |
| AHH | QVQLQQSGAEL | QVQL | GYTF | MHWV | IDPSD | NYNQK | AREE | CAREE | WFAYW | WGQG |
| 03733 | VKPGASVRLSC | QQSG | TSYW | KQRP | SYT | FKGKA | ITAW | ITAWF | GQGTL | TLVT |
| | KASGYTFTSYW | AELV | (SEQ | GQGL | (SEQ | TLAVD | FAY | AYW | VTVSA | VSA |
| | MHWVKQRPGQG | KPGA | ID | EWIG | ID | KSSST | (SEQ | (SEQ | (SEQ | (SEQ |
| | LEWIGEIDPSD | SVRL | NO: | E | NO: | AYMQL | ID | ID | ID | ID |
| | SYTNYNQKFKG | SCKA | 157) | (SEQ | 246) | SSLTS | NO: | NO: | NO: | NO: |
| | KATLAVDKSSS | S | | ID | | EDSAV | 324) | 372) | 392) | 421) |
| | TAYMQLSSLTS | (SEQ | | NO: | | YYC | | | | |
| | EDSAVYYCARE | ID | | 209) | | (SEQ | | | | |
| | EITAWFAYWGQ | NO: | | | | ID | | | | |
| | GTLVTVSA | 132) | | | | NO: | | | | |
| | (SEQ ID NO: | | | | | 282) | | | | |
| | 87) | | | | | | | | | |
| AHH | QVQLQQPGAEL | QVQL | GYTFT | MHWV | IHPGS | AYNQK | TRNG | CTRNG | WYFDV | WGAG |
| 03734 | VRPGASVKLSC | QQPG | DYE | KQTP | GGT | FKGKA | NGNW | NGNWY | WGAGT | TTLT |
| | KALGYTFTDYE | AELV | (SEQ | VHGL | (SEQ | TLTAD | YFDV | FDVW | TLTVS | VSS |
| | MHWVKQTPVHG | RPGA | ID | EWIG | ID | KSSST | (SEQ | (SEQ | S | (SEQ |
| | LEWIGAIHPGS | SVKL | NO: | A | NO: | AYMEL | ID | ID | (SEQ | ID |
| | GGTAYNQKFKG | SCKA | 174) | (SEQ | 247) | SSLTS | NO: | NO: | ID | NO: |
| | KATLTADKSSS | L | | ID | | EDSAV | 325) | 373) | NO: | 428) |
| | TAYMELSSLTS | (SEQ | | NO: | | YYC | | | 409) | |
| | EDSAVYYCTRN | ID | | 210) | | (SEQ | | | | |
| | GNGNWYFDVWG | NO: | | | | ID | | | | |
| | AGTTLTVSS | 133) | | | | NO: | | | | |
| | (SEQ ID NO: | | | | | 283) | | | | |
| | 88) | | | | | | | | | |
| AHH | QVQLQQSGAEL | QVQL | GYAF | MNWV | IYPGD | YYNGK | ARSG | CARSG | FAYWG | WGPG |
| 03737 | VRPGSSVKISC | QQSG | SSYW | KQRP | GDT | FKDKA | YRYD | YRYDA | PGTLV | TLVT |
| | KASGYAFSSYW | AELV | (SEQ | GQGL | (SEQ | TLTAD | AVFA | VFAYW | TVSA | VSA |
| | MNWVKQRPGQG | RPGS | ID | EWIG | ID | KSSST | Y | (SEQ | (SEQ | (SEQ |
| | LEWIGQIYPGD | SVKI | NO: | Q | NO: | AYMHL | (SEQ | ID | ID | ID |
| | GDTYYNGKFKD | SCKA | 175) | (SEQ | 248) | SSLTS | ID | NO: | NO: | NO: |
| | KATLTADKSSS | S | | ID | | EDSAV | NO: | 374) | 410) | 429) |
| | TAYMHLSSLTS | (SEQ | | NO: | | YFC | 326) | | | |
| | EDSAVYFCARS | ID | | 211) | | (SEQ | | | | |
| | GYRYDAVFAYW | NO: | | | | ID | | | | |
| | GPGTLVTVSA | 134) | | | | NO: | | | | |
| | (SEQ ID NO: | | | | | 284) | | | | |
| | 89) | | | | | | | | | |
| AHH | EVQLLESGGGL | EVQL | GFTF | MSWV | INSNG | YYPDS | ARGG | CARGG | YLGQG | LGQG |
| 03738 | VQPGGSLKLSC | LESG | SSYG | RQTP | GST | VKGRF | NPY | NPYL | TLVTV | TLVT |
| | AASGFTFSSYG | GGLV | (SEQ | DKRL | (SEQ | TISRD | (SEQ | (SEQ | SA | VSA |
| | MSWVRQTPDKR | QPGG | ID | ELVA | ID | NAKNT | ID | ID | (SEQ | (SEQ |
| | LELVATINSNG | SLKL | NO: | T | NO: | LYLQM | NO: | NO: | ID | ID |
| | GSTYYPDSVKG | SCAA | 165) | (SEQ | 240) | SSLKS | 327) | 375) | NO: | NO: |
| | RFTISRDNAKN | S | | ID | | EDTAM | | | 411) | 430) |
| | TLYLQMSSLKS | (SEQ | | NO: | | YYC | | | | |
| | EDTAMYYCARG | ID | | 200) | | (SEQ | | | | |
| | GNPYLGQGTLV | NO: | | | | ID | | | | |
| | TVSA (SEQ | 135) | | | | NO: | | | | |
| | ID NO: 90) | | | | | 274) | | | | |
| AHH | EVQLVETGGGL | EVQL | GFTFS | MSWV | INSDG | NYAPS | MRYG | | LLVLR | |
| 03739 | VQPGGSRGLSC | VETG | GFW | RQTP | SAI | IKDRF | SSYW | | C | |
| | EGSGFTFSGFW | GGLV | (SEQ | GKTL | (SEQ | TIFRD | YFD | | (SEQ | |
| | MSWVRQTPGKT | QPGG | ID | EWIG | ID | NDKST | (SEQ | | ID | |
| | LEWIGDINSDG | SRGL | NO: | D | NO: | LYLQM | ID | | NO: | |
| | SAINYAPSIKD | SCEG | 176) | (SEQ | 249) | SNVRS | NO: | | 412) | |
| | RFTIFRDNDKS | S | | ID | | EDTAT | 328) | | | |
| | TLYLQMSNVRS | (SEQ | | NO: | | YFC | | | | |
| | EDTATYFCMRY | ID | | 212) | | (SEQ | | | | |
| | GSSYWYFD | NO: | | | | ID | | | | |
| | (SEQ ID NO: | 136) | | | | NO: | | | | |
| | 91) | | | | | 285) | | | | |
| AHH | EVKIEESGGGL | EVKI | GFTFS | MSWV | IRLKS | HYAES | TRYY | CTRYY | WGQGT | WGQG |
| 03740 | VQPGGSMKLSC | EESG | SYW | RQSP | DNYAT | VKGKF | YGES | YGESW | LVTVS | TLVT |
| | VASGFTFSSYW | GGLV | (SEQ | EKGL | (SEQ | TISRD | (SEQ | (SEQ | S | VSS |
| | MSWVRQSPEKG | QPGG | ID | EWVA | ID | DSKSR | ID | ID | (SEQ | (SEQ |
| | LEWVAEIRLKS | SMKL | NO: | E | NO: | LYLQM | NO: | NO: | ID | ID |
| | DNYATHYAESV | SCVA | 177) | (SEQ | 250) | NSLRA | 329) | 376) | NO: | NO: |
| | KGKFTISRDDS | S | | ID | | EDTGI | | | 413) | 413) |
| | KSRLYLQMNSL | (SEQ | | NO: | | YYC | | | | |
| | RAEDTGIYYCT | ID | | 213) | | (SEQ | | | | |
| | RYYYGESWGQG | NO: | | | | ID | | | | |
| | TLVTVSS | 137) | | | | NO: | | | | |
| | (SEQ ID NO: | | | | | 286) | | | | |
| | 92) | | | | | | | | | |
| AHH | EVMLVESGGDL | EVML | GFTFS | MSWV | ISSGG | YYPDS | ARHY | CARHY | YWYFD | WGAG |
| 03742 | VKPGGSLKLSC | VESG | SYG | RQTP | SYT | VKGRF | YDYD | YDYDY | VWGAG | TTVT |
| | AASGFTFSSYG | GDLV | (SEQ | DKRL | (SEQ | TISRD | YWYF | WYFDV | TTVTV | VSS |
| | MSWVRQTPDKR | KPGG | ID | EWVA | ID | NAKNT | DV | W | SS | (SEQ |
| | LEWVATISSGG | SLKL | NO: | T | NO: | LYLQM | (SEQ | (SEQ | (SEQ | ID |
| | SYTYYPDSVKG | SCAA | 165) | (SEQ | 237) | SSLKS | ID | ID | ID | NO: |
| | RFTISRDNAKN | S | | ID | | EDTAM | NO: | NO: | NO: | 423) |
| | TLYLQMSSLKS | (SEQ | | NO: | | YYC | 330) | 377) | 400) | |
| | EDTAMYYCARH | ID | | 207) | | (SEQ | | | | |
| | YYDYDYWYFDV | NO: | | | | ID | | | | |
| | WGAGTTVTVSS | 138) | | | | NO: | | | | |
| | (SEQ ID NO: | | | | | 274) | | | | |
| | 93) | | | | | | | | | |
| AHH | QVQLQQPGSEL | QVQL | GYTFT | MHWV | IYPGS | NYDEK | TRSG | CTRSG | WYFDV | WGAG |
| 03743 | VRPGASVKLSC | QQPG | SYW | KQRY | GST | FKSKG | VEGL | VEGLL | WGAGT | TSLT |
| | KASGYTFTSYW | SELV | (SEQ | GQGL | (SEQ | TLTVD | LHWY | HWYFD | SLTVS | VSS |
| | MHWVKQRYGQG | RPGA | ID | EWIG | ID | TSSST | FDV | VW | S | (SEQ |
| | LEWIGNIYPGS | SVKL | NO: | N | NO: | AYMHL | (SEQ | (SEQ | (SEQ | ID |
| | GSTNYDEKFKS | SCKA | 157) | (SEQ | 251) | SSLTS | ID | ID | ID | NO: |
| | KGTLTVDTSSS | S | | ID | | EDSAV | NO: | NO: | NO: | 431) |
| | TAYMHLSSLTS | (SEQ | | NO: | | YYC | 331) | 378) | 414) | |
| | EDSAVYYCTRS | ID | | 214) | | (SEQ | | | | |
| | GVEGLLHWYFD | NO: | | | | ID | | | | |
| | VWGAGTSLTVS | 139) | | | | NO: | | | | |
| | S (SEQ ID | | | | | 287) | | | | |
| | NO: 94) | | | | | | | | | |
| AHH | EVKIEESGGGL | EVKI | GFTFS | MSWV | IRLKS | HYAES | TCDY | CTCDY | WFAYW | WGQG |
| 03746 | VQPGGSMKLSC | EESG | SYW | RQSP | DNYAT | VKGKF | DGGA | DGGAW | GQGTL | TLVT |
| | VASGFTFSSYW | GGLV | (SEQ | EKGL | (SEQ | TISRD | WFAY | FAYW | VTVSA | VSA |
| | MSWVRQSPEKG | QPGG | ID | EWVA | ID | DSKSR | (SEQ | (SEQ | (SEQ | (SEQ |
| | LEWVAEIRLKS | SMKL | NO: | E | NO: | LYLQM | ID | ID | ID | ID |
| | DNYATHYAESV | SCVA | 177) | (SEQ | 250) | NSLRA | NO: | NO: | NO: | NO: |
| | KGKFTISRDDS | S | | ID | | EDTGI | 332) | 379) | 392) | 421) |
| | KSRLYLQMNSL | (SEQ | | NO: | | YYC | | | | |
| | RAEDTGIYYCT | ID | | 213) | | (SEQ | | | | |
| | CDYDGGAWFAY | NO: | | | | ID | | | | |
| | WGQGTLVTVSA | 137) | | | | NO: | | | | |
| | (SEQ ID NO: | | | | | 286) | | | | |
| | 95) | | | | | | | | | |
| AHH | QIQLQQPGPEL | QIQL | GYTFT | MHW | IDPSN | RLNQK | ARCD | CARCD | GLDYW | WGQG |
| 03748 | VRPGASVKMSC | QQPG | SYW | VKQR | SET | FKDKA | GYYD | GYYDG | GQGTT | TTLT |
| | KASGYTFTSYW | PELV | (SEQ | PGQG | (SEQ | TLNVD | GLDY | LDYW | LTVSS | VSS |
| | MHWVKQRPGQG | RPGA | ID | LEWI | ID | KSSNT | (SEQ | (SEQ | (SEQ | (SEQ |
| | LEWIGMIDPSN | SVKM | NO: | GM | NO: | ACMQL | ID | ID | ID | ID |
| | SETRLNQKFKD | SCKA | 157) | (SEQ | 252) | SSLTS | NO: | NO: | NO: | NO: |
| | KATLNVDKSSN | S | | ID NO: | | EDSAV | 333) | 380) | 415) | 425) |
| | TACMQLSSLTS | (SEQ | | 215) | | YYC | | | | |
| | EDSAVYYCARC | ID | | | | (SEQ | | | | |
| | DGYYDGLDYWG | NO: | | | | ID | | | | |
| | QGTTLTVSS | 140) | | | | NO: | | | | |
| | (SEQ ID NO: | | | | | 288) | | | | |
| | 96) | | | | | | | | | |
| AHH | QVQLQQPGAEL | QVQL | GYTFT | IHWV | INPSS | NYNQK | AREG | CAREG | WYFDV | WGAG |
| 03749 | ARPGASVKMSC | QQPG | SYT | KQRP | GYT | FKDKA | KNWY | KNWYF | WGAGT | TTVT |
| | KASGYTFTSYT | AELA | (SEQ | GQGL | (SEQ | TLTAD | FDV | DVW | TVTVS | VSS |
| | IHWVKQRPGQG | RPGA | ID | EWIG | ID | KSSST | (SEQ | (SEQ | S | (SEQ |
| | LEWIGYINPSS | SVKM | NO: | (SEQ | NO: | AYMQL | ID | ID | (SEQ | ID |
| | GYTNYNQKFKD | SCKA | 162) | ID | 236) | SSLTS | NO: | NO: | ID | NO: |
| | KATLTADKSSS | S | | NO: | | EDSAV | 334) | 381) | NO: | 423) |
| | TAYMQLSSLTS | (SEQ | | 196) | | YYC | | | 416) | |
| | EDSAVYYCARE | ID | | | | (SEQ | | | | |
| | GKNWYFDVWGA | NO: | | | | ID | | | | |
| | GTTVTVSS | 141) | | | | NO: | | | | |
| | (SEQ ID NO: | | | | | 270) | | | | |
| | 97) | | | | | | | | | |
| AHH | QIQLQQSGAEL | QIQL | GYTFT | MHWI | INPSN | NYNEK | ARRI | CARRI | TLDYW | WGQG |
| 03750 | VRPGVSVKLSC | QQSG | SYW | KQRP | GGT | FKSKA | YRTL | YRTLD | GQGTT | TTVT |
| | KASGYTFTSYW | AELV | (SEQ | EQGL | (SEQ | TLTVD | DY | YW | VTVSS | VSS |
| | MHWIKQRPEQG | RPGV | ID | ERIG | ID | KSSST | (SEQ | (SEQ | (SEQ | (SEQ |
| | LERIGEINPSN | SVKL | NO: | E | NO: | AYMQL | ID | ID | ID | ID |
| | GGTNYNEKFKS | SCKA | 157) | (SEQ | 231) | SSLTS | NO: | NO: | NO: | NO: |
| | KATLTVDKSSS | S | | ID | | EDSAV | 335) | 382) | 417) | 422) |
| | TAYMQLSSLTS | (SEQ | | NO: | | YYC | | | | |
| | EDSAVYYCARR | ID | | 216) | | (SEQ | | | | |
| | IYRTLDYWGQG | NO: | | | | ID | | | | |
| | TTVTVSS | 142) | | | | NO: | | | | |
| | (SEQ ID NO: | | | | | 289) | | | | |
| | 98) | | | | | | | | | |
| AHH | EVQLQQSGAEL | EVQL | GYTFT | MHWV | IYPGN | AYNQ | TRSG | CTRSG | WFAYW | WGQG |
| 03751 | VKPGASVKMSC | QQSG | SYN | KQTP | GDT | KFKGK | GNLW | GNLWF | GQGTL | TLVT |
| | KASGYTFTSYN | AELV | (SEQ | GQGL | (SEQ | ATVTA | FAY | AYW | VTVSA | VSA |
| | MHWVKQTPGQG | KPGA | ID | EWIG | ID | DRSSS | (SEQ | (SEQ | (SEQ | (SEQ |
| | LEWIGVIYPGN | SVKM | NO: | V | NO: | TAYMQ | ID | ID | ID | ID |
| | GDTAYNQKFKG | SCKA | 178) | (SEQ | 253) | LSSLT | NO: | NO: | NO: | NO: |
| | KATVTADRSSS | S | | ID | | SEDSA | 336) | 383) | 392) | 421) |
| | TAYMQLSSLTS | (SEQ | | NO: | | VYYC | | | | |
| | EDSAVYYCTRS | ID | | 217) | | (SEQ | | | | |
| | GGNLWFAYWGQ | NO: | | | | ID | | | | |
| | GTLVTVSA | 143) | | | | NO: | | | | |
| | (SEQ ID NO: | | | | | 290) | | | | |
| | 99) | | | | | | | | | |
| AHH | QIQLQQSGAEL | QIQL | GYTFS | IEWV | ILPGS | NYNEK | ARRT | CARRT | WFAYW | WGQG |
| 03752 | MKPGASVKISC | QQSG | SYW | KQRP | GST | FKGKA | YYGN | YYGNA | GQGTL | TLVT |
| | KATGYTFSSYW | AELM | (SEQ | GHGL | (SEQ | TFTAD | AWFA | WFAYW | VTVSA | VSA |
| | IEWVKQRPGHG | KPGA | ID | EWIG | ID | TSSNT | Y | (SEQ | (SEQ | (SEQ |
| | LEWIGEILPGS | SVKI | NO: | E | NO: | AYMQL | (SEQ | ID | ID | ID |
| | GSTNYNEKFKG | SCKA | 179) | (SEQ | 254) | SSLTS | ID | NO: | NO: | NO: |
| | KATFTADTSSN | T | | ID | | EDSAV | NO: | 384) | 392) | 421) |
| | TAYMQLSSLTS | (SEQ | | NO: | | YYC | 337) | | | |
| | EDSAVYYCARR | ID | | 218) | | (SEQ | | | | |
| | TYYGNAWFAYW | NO: | | | | ID | | | | |
| | GQGTLVTVSA | 144) | | | | NO: | | | | |
| | (SEQ ID NO: | | | | | 291) | | | | |
| | 100) | | | | | | | | | |
| AHH | QVKLVESGGGL | QVKL | GFTFT | MSWV | IRNKA | EYSAS | ARDK | CARDK | WFAYW | WGQG |
| 03753 | VQPGGSLRLSC | VESG | DYY | RQPP | NGYTT | VKGRF | RITT | RITTV | GQGTL | TLVT |
| | ATSGFTFTDYY | GGLV | (SEQ | GKAL | (SEQ | TISRD | VEAW | EAWFA | VTVSA | VSA |
| | MSWVRQPPGKA | QPGG | ID | EWLG | ID | NSQSI | FAY | YW | (SEQ | (SEQ |
| | LEWLGFIRNKA | SLRL | NO: | F | NO: | LYLQM | (SEQ | (SEQ | ID | ID |
| | NGYTTEYSASV | SCAT | 180) | (SEQ | 255) | NTLRA | ID | ID | NO: | NO: |
| | KGRFTISRDNS | S | | ID | | EDSAT | NO: | NO: | 392) | 421) |
| | QSILYLQMNTL | (SEQ | | NO: | | YYC | 338) | 385) | | |
| | RAEDSATYYCA | ID | | 219) | | (SEQ | | | | |
| | RDKRITTVEAW | NO: | | | | ID | | | | |
| | FAYWGQGTLVT | 145) | | | | NO: | | | | |
| | VSA (SEQ ID | | | | | 292) | | | | |
| | NO: 101) | | | | | | | | | |
| AHH | EVKLVETGGGL | EVKL | GFTFS | MSWV | ISSGG | YYPDS | ARGY | CARGY | SFAYW | WGQG |
| 03754 | VKPGGSLKLSC | VETG | SYA | RQTP | ST | VKGRF | GSSF | GSSFA | GQGTP | TPVT |
| | AASGFTFSSYA | GGLV | (SEQ | EKRL | (SEQ | TISRD | AY | YW | VTVSS | VSS |
| | MSWVRQTPEKR | KPGG | ID | EWVA | ID | NARNI | (SEQ | (SEQ | (SEQ | (SEQ |
| | LEWVASISSGG | SLKL | NO: | S | NO: | LYLQM | ID | ID | ID | ID |
| | STYYPDSVKGR | SCAA | 151) | (SEQ | 224) | SSLRS | NO: | NO: | NO: | NO: |
| | FTISRDNARNI | S | | ID | | EDTAM | 339) | 386) | 418) | 398) |
| | LYLQMSSLRSE | (SEQ | | NO: | | YYC | | | | |
| | DTAMYYCARGY | ID | | 184) | | (SEQ | | | | |
| | GSSFAYWGQGT | NO: | | | | ID | | | | |
| | PVTVSS (SEQ | 146) | | | | NO: | | | | |
| | ID NO: 102) | | | | | 259) | | | | |
| AHH | QVQLQQSGAEL | QVQL | GYTFT | MYWV | INPSN | NFNEK | TRGG | CTRGG | WFAYW | WGQG |
| 03755 | VKPGASVKLSC | QQSG | SYY | KQRP | SGT | FKSKA | DYDA | DYDAS | GQGTL | TLVT |
| | KASGYTFTSYY | AELV | (SEQ | GQGL | (SEQ | TLTVD | SWFA | WFAYW | VTVSA | VSA |
| | MYWVKQRPGQG | KPGA | ID | EWIG | ID | KSSST | Y | (SEQ | (SEQ | (SEQ |
| | LEWIGEINPSN | SVKL | NO: | E | NO: | AYMQL | (SEQ | ID | ID | ID |
| | SGTNFNEKFKS | SCKA | 158) | (SEQ | 256) | SSLTS | ID | NO: | NO: | NO: |
| | KATLTVDKSSS | S | | ID | | EDSAV | NO: | 387) | 392) | 421) |
| | TAYMQLSSLTS | (SEQ | | NO: | | YYC | 340) | | | |
| | EDSAVYYCTRG | ID | | 191) | | (SEQ | | | | |
| | GDYDASWFAYW | NO: | | | | ID | | | | |
| | GQGTLVTVSA | 115) | | | | NO: | | | | |
| | (SEQ ID NO: | | | | | 266) | | | | |
| | 103) | | | | | | | | | |
| AHH | QVQMKESGPEP | QVQM | GYTLT | VSWV | IYPGS | YYNEK | ARRT | CARRT | FDYWG | WGQG |
| 03756 | VKPGASVKMSC | KESG | DYV | KQRL | GST | FKDKV | ARAF | ARAFD | QGTTV | TTVT |
| | RASGYTLTDYV | PEPV | (SEQ | GQGL | (SEQ | TLTAD | DY | YW | TVSS | VSS |
| | VSWVKQRLGQG | KPGA | ID | EWIG | ID | TSSNT | (SEQ | (SEQ | (SEQ | (SEQ |
| | LEWIGEIYPGS | SVKM | NO: | E | NO: | VHIQL | ID | ID | ID | ID |
| | GSTYYNEKFKD | SCRA | 181) | (SEQ | 251) | SSLTS | NO: | NO: | NO: | NO: |
| | KVTLTADTSSN | S | | ID | | EDSAV | 341) | 388) | 419) | 422) |
| | TVHIQLSSLTS | (SEQ | | NO: | | YFC | | | | |
| | EDSAVYFCARR | ID | | 220) | | (SEQ | | | | |
| | TARAFDYWGQG | NO: | | | | ID | | | | |
| | TTVTVSS | 147) | | | | NO: | | | | |
| | (SEQ ID NO: | | | | | 293) | | | | |
| | 104) | | | | | | | | | |
| AHH | EVKLVESGGGL | EVKL | GFTFS | MSWV | ISSGG | YYPDS | ARRI | CARRI | WFAYW | WGQG |
| 03757 | VKPGGSLKLSC | VESG | SYA | RQTP | SYT | VKGRF | GYDG | GYDGG | GQGTL | TLVT |
| | AASGFTFSSYA | GGLV | (SEQ | EKRL | (SEQ | TISRD | GGSW | GSWFA | VTVSA | VSA |
| | MSWVRQTPEKR | KPGG | ID | EWVA | ID | NAKNT | FAY | YW | (SEQ | (SEQ |
| | LEWVATISSGG | SLKL | NO: | T | NO: | LYLQM | (SEQ | (SEQ | ID | ID |
| | SYTYYPDSVKG | SCAA | 151) | (SEQ | 237) | SSLRS | ID | ID | NO: | NO: |
| | RFTISRDNAKN | S | | ID | | EDTAM | NO: | NO: | 392) | 421) |
| | TLYLQMSSLRS | (SEQ | | NO: | | YYC | 342) | 389) | | |
| | EDTAMYYCARR | ID | | 221) | | (SEQ | | | | |
| | IGYDGGGSWFA | NO: | | | | ID | | | | |
| | YWGQGTLVTVS | 148) | | | | NO: | | | | |
| | A (SEQ ID | | | | | 294) | | | | |
| | NO: 105) | | | | | | | | | |
| AHH | EVKIVESGGGL | EVKI | GFTFN | MNWV | IRSKS | YYADS | CERV | LCERV | CVLGP | WGQG |
| 03758 | VQPKGSLKLSC | VESG | TYA | RQAP | NNYVT | LKDRF | RRCV | RRCVL | RDSGH | TLVT |
| | AASGFTFNTYA | GGLV | (SEQ | EKGL | (SEQ | TISRD | (SEQ | (SEQ | CLC | VSA |
| | MNWVRQAPEKG | QPKG | ID | EWVA | ID | DSQSM | ID | ID | (SEQ | (SEQ |
| | LEWVARIRSKS | SLKL | NO: | R | NO: | LYLQM | NO: | NO: | ID | ID |
| | NNYVTYYADSL | SCAA | 182) | (SEQ | 257) | NNLKT | 343) | 390) | NO: | NO: |
| | KDRFTISRDDS | S | | ID | | EDTCH | | | 420) | 421) |
| | QSMLYLQMNNL | (SEQ | | NO: | | VLL | | | | |
| | KTEDTCHVLLC | ID | | 222) | | (SEQ | | | | |
| | ERVRRCVLGPR | NO: | | | | ID | | | | |
| | DSGHCLC | 149) | | | | NO: | | | | |
| | (SEQ ID NO: | | | | | 295) | | | | |
| | 106) | | | | | | | | | |
| AHH | QVQLVETGGGL | QVQL | GFTFS | MHWL | ITVKS | NYAES | SRWF | CSRWF | WFAYW | WGQG |
| 03759 | VRPGNSLKLSC | VETG | NYR | RQPP | DNYGA | VKGRF | AY | AYW | GQGTL | TLVT |
| | VTSGFTFSNYR | GGLV | (SEQ | GKRL | (SEQ | TISRD | (SEQ | (SEQ | VTVSA | VSA |
| | MHWLRQPPGKR | RPGN | ID | EWIA | ID | DSKSS | ID | ID | (SEQ | (SEQ |
| | LEWIAVITVKS | SLKL | NO: | V | NO: | VYLQM | NO: | NO: | ID | ID |
| | DNYGANYAESV | SCVT | 183) | (SEQ | 258) | NRLRE | 344) | 391) | NO: | NO: |
| | KGRFTISRDDS | S | | ID | | EDTAT | | | 392) | 421) |
| | KSSVYLQMNRL | (SEQ | | NO: | | YYC | | | | |
| | REEDTATYYCS | ID | | 223) | | (SEQ | | | | |
| | RWFAYWGQGTL | NO: | | | | ID | | | | |
| | VTVSA (SEQ | 150) | | | | NO: | | | | |
| | ID NO: 107) | | | | | 296) | | | | |

TABLE 2B
Exemplary Clones-Heavy Chain Sequences

| | | | | | | | | JUNC- | J- | |
|---|---|---|---|---|---|---|---|---|---|---|
| ID | V-D-J-REGION | H-FR1 | CDRH1 | H-FR2 | CDRH2 | H-FR3 | CDRH3 | TION | REGION | H-FR4 |
| AHH | QVQLQQSGAE | QVQL | GYTFT | MHW | IHPGS | AYNQ | TRSDY | CTRSD | FAYWGQ | WGQG |
| 03760 | LVRPGASVKL | QQSG | DYE | VKQT | GGT | KFKG | GSSYE | YGSSY | GTLVTV | TLVT |
| | SCKALGYTFT | AELVR | (SEQ ID | PVHG | (SEQ ID | KATL | FAY | EFAYW | SS (SEQ | VSS |
| | DYEMHWVKQ | PGASV | NO: | LGWI | NO: | TADK | (SEQ ID | (SEQ ID | ID NO: | (SEQ |
| | TPVHGLGWIG | KLSCK | 174) | GA | 247) | SSSTA | NO: | NO: | 399) | ID NO: |
| | AIHPGSGGTAY | AL | | (SEQ | | YMEL | 531) | 558) | | 413) |
| | NQKFKGKATL | (SEQ | | ID NO: | | SSLTS | | | | |
| | TADKSSSTAY | ID NO: | | 493) | | EDSA | | | | |
| | MELSSLTSEDS | 461) | | | | VYYC | | | | |
| | AVYYCTRSDY | | | | | (SEQ | | | | |
| | GSSYEFAYWG | | | | | ID NO: | | | | |
| | QGTLVTVSS | | | | | 283) | | | | |
| | (SEQ ID NO: | | | | | | | | | |
| | 432) | | | | | | | | | |
| AHH | QIQLKESGPGL | QIQLK | GYSITS | WNWI | ISYSGS | SYNPS | ARSRG | CARSR | YFDYWG | WGQG |
| 03765 | VKPSQSLSLTC | ESGPG | DYA | RQFP | T (SEQ | LKSRI | NYFDY | GNYFD | QGTTVT | TTVT |
| | TVTGYSITSDY | LVKPS | (SEQ ID | GNKL | ID NO: | SITRD | (SEQ ID | YW | VSS | VSS |
| | AWNWIRQFPG | QSLSL | NO: | EWM | 506) | TSKN | NO: | (SEQ ID | (SEQ ID | (SEQ |
| | NKLEWMGYIS | TCTVT | 482) | GY | | QFFL | 532) | NO: | NO: 397) | ID NO: |
| | YSGSTSYNPSL | (SEQ | | (SEQ | | QLNS | | 559) | | 422) |
| | KSRISITRDTSK | ID NO: | | ID NO: | | VTTE | | | | |
| | NQFFLQLNSVT | 462) | | 494) | | DTAT | | | | |
| | TEDTATYYCA | | | | | YYC | | | | |
| | RSRGNYFDYW | | | | | (SEQ | | | | |
| | GQGTTVTVSS | | | | | ID NO: | | | | |
| | (SEQ ID NO: | | | | | 516) | | | | |
| | 433) | | | | | | | | | |
| AHH | QIQLQQSGAEL | QIQLQ | GYTFTS | IHWV | INPSSG | NYNQ | ARSGL | CARSG | WFAYW | WGQG |
| 03767 | ARPGASVRMS | QSGAE | YT | KQRP | YT | KFKD | RQAW | LRQA | GQGTLV | TLVT |
| | CKASGYTFTS | LARPG | (SEQ ID | GQGL | (SEQ ID | KATL | FAY | WFAY | TVSA | VSA |
| | YTIHWVKQRP | ASVR | NO: | EWIG | NO: | TADK | (SEQ ID | W (SEQ | (SEQ ID | (SEQ |
| | GQGLEWIGYIN | MSCK | 162) | (SEQ | 236) | SSSTA | NO: | ID NO: | NO: 392) | ID NO: |
| | PSSGYTNYNQ | AS | | ID NO: | | YMQL | 533) | 560) | | 421) |
| | KFKDKATLTA | (SEQ | | 196) | | SSLTS | | | | |
| | DKSSSTAYMQ | ID NO: | | | | EDSA | | | | |
| | LSSLTSEDSAV | 463) | | | | VYYC | | | | |
| | YYCARSGLRQ | | | | | (SEQ | | | | |
| | AWFAYWGQG | | | | | ID NO: | | | | |
| | TLVTVSA (SEQ | | | | | 270) | | | | |
| | ID NO: 434) | | | | | | | | | |
| AHH | QVQLVETGGG | QVQL | GFTFSN | MHW | ITVKS | NYAE | SRLFA | CSRLF | FAYWGQ | WGQG |
| 03768 | LVRPGNSLKLS | VETG | YR | LRQP | DNYG | SVKG | Y (SEQ | AYW | GTLVTV | TLVT |
| | CVTSGFTFSNY | GGLV | (SEQ ID | LGKR | A (SEQ | RFTIS | ID NO: | (SEQ ID | SA (SEQ | VSA |
| | RMHWLRQPLG | RPGNS | NO: | LEWI | ID NO: | RDDS | 534) | NO: | ID NO: | (SEQ |
| | KRLEWIAVITV | LKLSC | 183) | AV | 258) | KSSV | | 561) | 395) | ID NO: |
| | KSDNYGANYA | VTS | | (SEQ | | YLQM | | | | 421) |
| | ESVKGRFTISR | (SEQ | | ID NO: | | NRLR | | | | |
| | DDSKSSVYLQ | ID NO: | | 495) | | EEDT | | | | |
| | MNRLREEDTA | 150) | | | | ATYY | | | | |
| | TYYCSRLFAY | | | | | C | | | | |
| | WGQGTLVTVSA | | | | | (SEQ | | | | |
| | (SEQ ID NO: | | | | | ID NO: | | | | |
| | 435) | | | | | 296) | | | | |
| AHH | QVQLQQPGAE | QVQL | GYTFTS | MHW | INPSN | NYNE | TITGFD | CTITGF | FDVWGA | WGAG |
| 03770 | LVKPGASVKL | QQPG | YW | VKLR | GGT | KFKR | V (SEQ | DVW | GTTVTV | TTVT |
| | SCKASGYTFTS | AELV | (SEQ ID | PGQG | (SEQ ID | KATL | ID NO: | (SEQ ID | SS (SEQ | VSS |
| | YWMHWVKLR | KPGAS | NO: | FEWI | NO: | TVDK | 535) | NO: | ID NO: | (SEQ |
| | PGQGFEWIGEI | VKLSC | 157) | GE | 231) | SSSTA | | 562) | 585) | ID NO: |
| | NPSNGGTNYN | KAS | | (SEQ | | YMQL | | | | 423) |
| | EKFKRKATLT | (SEQ | | ID NO: | | SSLTS | | | | |
| | VDKSSSTAYM | ID NO: | | 496) | | EDSA | | | | |
| | QLSSLTSEDSA | 464) | | | | VYYC | | | | |
| | VYYCTITGFDV | | | | | (SEQ | | | | |
| | WGAGTTVTVSS | | | | | ID NO: | | | | |
| | (SEQ ID NO: | | | | | 517) | | | | |
| | 436) | | | | | | | | | |
| AHH | QIQLQQSGAEL | QIQLQ | GYTFT | IQWV | IFPGTG | YYNE | ARGGY | CARGG | FAYWGQ | WGQG |
| 03771 | VKPGASVKLS | QSGAE | TYW | KQRP | TT | KFKG | YNSSP | YYNSS | GTLVTV | TLVT |
| | CKTSGYTFTTY | LVKPG | (SEQ ID | GQGL | (SEQ ID | KATL | FAY | PFAYW | SA (SEQ | VSA |
| | WIQWVKQRPG | ASVKL | NO: | GWIG | NO: | TIDTS | (SEQ ID | (SEQ ID | ID NO: | (SEQ |
| | QGLGWIGEIFP | SCKTS | 483) | E (SEQ | 507) | SSTA | NO: | NO: | 395) | ID NO: |
| | GTGTTYYNEK | (SEQ | | ID NO: | | YMQL | 536) | 563) | | 421) |
| | FKGKATLTIDT | ID NO: | | 497) | | SSLTS | | | | |
| | SSSTAYMQLSS | 465) | | | | EDSA | | | | |
| | LTSEDSAVYFC | | | | | VYFC | | | | |
| | ARGGYYNSSP | | | | | (SEQ | | | | |
| | FAYWGQGTLV | | | | | ID NO: | | | | |
| | TVSA (SEQ ID | | | | | 518) | | | | |
| | NO: 437) | | | | | | | | | |
| AHH | DVQLVESGGG | DVQL | GFTFTD | MSWV | IRNKA | EYSA | ARDGE | CARDG | AYWGQ | WGQG |
| 03772 | LVQPGGSLRLS | VESGG | YY | RQPP | NGYTT | SVKG | VRRAL | EVRRA | GTLVTV | TLVT |
| | CATSGFTFTDY | GLVQP | (SEQ ID | GKAL | (SEQ ID | RFTIS | AY | LAYW | SA (SEQ | VSA |
| | YMSWVRQPPG | GGSLR | NO: | EWLG | NO: | RDNS | (SEQ ID | (SEQ ID | ID NO: | (SEQ |
| | KALEWLGFIR | LSCAT | 180) | F (SEQ | 255) | QSILY | NO: | NO: | 394) | ID NO: |
| | NKANGYTTEY | S (SEQ | | ID NO: | | LQMN | 537) | 564) | | 421) |
| | SASVKGRFTIS | ID NO: | | 219) | | TLRA | | | | |
| | RDNSQSILYLQ | 466) | | | | EDSA | | | | |
| | MNTLRAEDSA | | | | | TYYC | | | | |
| | TYYCARDGEV | | | | | (SEQ | | | | |
| | RRALAYWGQ | | | | | ID NO: | | | | |
| | GTLVTVSA | | | | | 292) | | | | |
| | (SEQ ID NO: | | | | | | | | | |
| | 438) | | | | | | | | | |
| AHH | QVQLQQPAAE | QVQL | GYTFTS | MHW | INPSSG | EYNQ | VRHYY | CVRHY | YFDYWG | WGQG |
| 03773 | LARPGASVKM | QQPA | ST (SEQ | VKQR | YT | KFKD | FDY | YFDY | QGTTVT | TTVT |
| | SCKASGYTFTS | AELAR | ID NO: | PGQG | (SEQ ID | KTTL | (SEQ ID | W (SEQ | VSS (SEQ | VSS |
| | STMHWVKQRP | PGASV | 484) | LEWI | NO: | TADK | NO: | ID NO: | ID NO: | (SEQ |
| | GQGLEWIGYIN | KMSC | | GY | 236) | SSSTA | 538) | 565) | 397) | ID NO: |
| | PSSGYTEYNQ | KAS | | (SEQ | | YMQL | | | | 422) |
| | KFKDKTTLTA | (SEQ | | ID NO: | | SSLTS | | | | |
| | DKSSSTAYMQ | ID NO: | | 190) | | EDSA | | | | |
| | LSSLTSEDSAV | 467) | | | | VYYC | | | | |
| | YYCVRHYYFD | | | | | (SEQ | | | | |
| | YWGQGTTVTV | | | | | ID NO: | | | | |
| | SS (SEQ ID | | | | | 519) | | | | |
| | NO: 439) | | | | | | | | | |
| AHH | EVMLVESGGG | EVML | GFTFSS | MSWV | ISSGG | YYLD | TRVSA | CTRVS | YFDVWG | WGAG |
| 03774 | LVKPGGSLKIS | VESGG | YT | RQNP | GYT | SVKG | KYFDV | AKYFD | AGTTLT | TTLT |
| | CAASGFTFSSY | GLVKP | (SEQ ID | EKRL | (SEQ ID | RFTPS | (SEQ ID | VW | VSS (SEQ | VSS |
| | TMSWVRQNPE | GGSLK | NO: | EWVA | NO: | RDNG | NO: | (SEQ ID | ID NO: | (SEQ |
| | KRLEWVATISS | ISCAA | 485) | T(SEQ | 508) | KNTL | 539) | NO: | 586) | ID NO: |
| | GGGYTYYLDS | S (SEQ | | ID NO: | | NLQM | | 566) | | 428) |
| | VKGRFTPSRD | ID NO: | | 498) | | SSLKS | | | | |
| | NGKNTLNLQM | 468) | | | | EDTA | | | | |
| | SSLKSEDTAM | | | | | MYYC | | | | |
| | YYCTRVSAKY | | | | | (SEQ | | | | |
| | FDVWGAGTTL | | | | | ID NO: | | | | |
| | TVSS (SEQ | | | | | 520) | | | | |
| | ID NO: 440) | | | | | | | | | |
| AHH | QVQLQQSGAE | QVQL | GYTFT | LYWV | INPSN | NFNE | TRSYY | CTRSY | DWYFDV | WGAG |
| 03776 | LVKPGASVKL | QQSG | NFY | KQRP | GGT | KFKS | DYDW | YDYD | WGAGTT | TTVT |
| | SCKASGYTFT | AELV | (SEQ ID | GQGL | (SEQ ID | KATL | YFDV | WYFD | VTVSS | VSS |
| | NFYLYWVKQR | KPGAS | NO: | EWIG | NO: | TVDK | (SEQ ID | VW | (SEQ ID | (SEQ |
| | PGQGLEWIGGI | VKLSC | 486) | G | 231) | SSSTA | NO: | (SEQ ID | NO: 587) | ID NO: |
| | NPSNGGTNFN | KAS | | (SEQ | | YMQL | 540) | NO: | | 423) |
| | EKFKSKATLT | (SEQ | | ID NO: | | SSLTS | | 567) | | |
| | VDKSSSTAYM | ID NO: | | 499) | | EDSA | | | | |
| | QLSSLTSEDSA | 115) | | | | VYYC | | | | |
| | VYYCTRSYYD | | | | | (SEQ | | | | |
| | YDWYFDVWG | | | | | ID NO: | | | | |
| | AGTTVTVSS | | | | | 266) | | | | |
| | (SEQ ID NO: | | | | | | | | | |
| | 441) | | | | | | | | | |
| AHH | QVQLQQSGAE | QVQL | GYTFTS | INWV | IYPSDS | NYNQ | TRQNY | CTRQN | WYFDV | WGAG |
| 03777 | LVRPGASVKL | QQSG | YW | KQRP | YT | KFKD | YGSSH | YYGSS | WGAGTT | TTLT |
| | SCKASGYTFTS | AELVR | (SEQ ID | GQGL | (SEQ ID | KATL | WYFD | HWYF | LTVSS | VSS |
| | YWINWVKQRP | PGASV | NO: | EWIG | NO: | TVDK | V (SEQ | DVW | (SEQ ID | (SEQ |
| | GQGLEWIGNIY | KLSCK | 157) | N | 509) | SSSTA | ID NO: | (SEQ ID | NO: 409) | ID NO: |
| | PSDSYTNYNQ | AS | | (SEQ | | YMQL | 541) | NO: | | 428) |
| | KFKDKATLTV | (SEQ | | ID NO: | | SSPTS | | 568) | | |
| | DKSSSTAYMQ | ID NO: | | 500) | | EDSA | | | | |
| | LSSPTSEDSAV | 113) | | | | VYYC | | | | |
| | YYCTRQNYYG | | | | | (SEQ | | | | |
| | SSHWYFDVW | | | | | ID NO: | | | | |
| | GAGTTLTVSS | | | | | 521) | | | | |
| | (SEQ ID NO: | | | | | | | | | |
| | 442) | | | | | | | | | |
| AHH | QVQLQQPGAE | QVQL | GYTFTS | MNW | IDPSDS | HYNQ | ANWA | CANW | WFAYW | WGQG |
| 03778 | LVKPGAPVKL | QQPG | YW | VKQR | ET | KFKD | WFAY | AWFA | GQGTLV | TLVT |
| | SCKASGYTFTS | AELV | (SEQ ID | PGRG | (SEQ ID | KATL | (SEQ ID | YW | TVSS | VSS |
| | YWMNWVKQR | KPGAP | NO: | LEWI | NO: | TVDK | NO: | (SEQ ID | (SEQ ID | (SEQ |
| | PGRGLEWIGRI | VKLSC | 157) | GR | 229) | SSSTA | 317) | NO: | NO: 588) | ID NO: |
| | DPSDSETHYN | KAS | | (SEQ | | YIQLS | | 365) | | 413) |
| | QKFKDKATLT | (SEQ | | ID NO: | | SLTSE | | | | |
| | VDKSSSTAYIQ | ID NO: | | 204) | | DSAV | | | | |
| | LSSLTSEDSAV | 469) | | | | YYC | | | | |
| | YYCANWAWF | | | | | (SEQ | | | | |
| | AYWGQGTLVT | | | | | ID NO: | | | | |
| | VSS (SEQ ID | | | | | 276) | | | | |
| | NO: 443) | | | | | | | | | |
| AHH | QIQFAQSGPEL | QIQFA | GYTFT | MHW | INTET | TYAD | ASFYY | CASFY | YFDYRG | RGQG |
| 03779 | KKPGETVKISC | QSGPE | DYS | VKQA | GEP | DFKG | GNFAY | YGNFA | QGTTLT | TTLT |
| | KAFGYTFTDY | LKKPG | (SEQ ID | PGKG | (SEQ ID | RFAFS | YFDY | YYFDY | VSS | VSS |
| | SMHWVKQAP | ETVKI | NO: | LKW | NO: | LETSA | (SEQ ID | R(SEQ | (SEQ ID | (SEQ |
| | GKGLKWMGW | SCKAF | 487) | MGW | 225) | STAY | NO: | ID NO: | NO: 589) | ID NO: |
| | INTETGEPTYA | (SEQ | | (SEQ | | LQINN | 542) | 569) | | 597) |
| | DDFKGRFAFSL | ID NO: | | ID NO: | | LKNE | | | | |
| | ETSASTAYLQI | 470) | | 185) | | DTAT | | | | |
| | NNLKNEDTAT | | | | | YFC | | | | |
| | YFCASFYYGN | | | | | (SEQ | | | | |
| | FAYYFDYRGQ | | | | | ID NO: | | | | |
| | GTTLTVSS | | | | | 260) | | | | |
| | (SEQ ID NO: | | | | | | | | | |
| | 444) | | | | | | | | | |
| AHH | EVKIEESGGGL | EVKIE | GFTFSN | MNW | IRLKS | YYAE | TRIYD | CTRIY | WYFDV | WGAG |
| 03780 | VQPGGAMKLS | ESGGG | YW | VRQS | NNYAT | SVKG | SGSSY | DSGSS | WGAGTT | TTVT |
| | CVASGFTFSNY | LVQPG | (SEQ ID | PEKG | (SEQ ID | RFTIS | TWYFD | YTWYF | VTVSS | VSS |
| | WMNWVRQSP | GAMK | NO: | LEWV | NO: | RDDS | V (SEQ | DVW | (SEQ ID | (SEQ |
| | EKGLEWVAEI | LSCVA | 166) | AE | 241) | QSSV | ID NO: | (SEQ ID | NO: 416) | ID NO: |
| | RLKSNNYATY | S (SEQ | | (SEQ | | YLQM | 543) | NO: | | 423) |
| | YAESVKGRFTI | ID NO: | | ID NO: | | NDLR | | 570) | | |
| | SRDDSQSSVYL | 471) | | 201) | | TEDT | | | | |
| | QMNDLRTEDT | | | | | GIYY | | | | |
| | GIYYCTRIYDS | | | | | C | | | | |
| | GSSYTWYFDV | | | | | (SEQ | | | | |
| | WGAGTTVTVS | | | | | ID NO: | | | | |
| | S (SEQ ID | | | | | 522) | | | | |
| | NO: 445) | | | | | | | | | |
| AHH | QVILKESGPGI | QVILK | GFSLST | VGWI | IWWN | YYNP | ARIGG | CARIG | YWYFDV | WGAG |
| 03782 | LQPSQTLSLTC | ESGPG | SGMS | RQPS | DDK | ALKS | NDGY | GNDG | WGAGTS | TSLTV |
| | SFSGFSLSTSG | ILQPS | (SEQ ID | GKGL | (SEQ ID | RLTIS | YWYF | YYWY | LTVSS | SS |
| | MSVGWIRQPS | QTLSL | NO: | EWLA | NO: | KDTP | DV | FDVW | (SEQ ID | (SEQ |
| | GKGLEWLAHI | TCSFS | 163) | H | 238) | NNQV | (SEQ ID | (SEQ ID | NO: 590) | ID NO: |
| | WWNDDKYYN | (SEQ | | (SEQ | | FLKIA | NO: | NO: | | 431) |
| | PALKSRLTISK | ID NO: | | ID NO: | | SVVT | 311) | 359) | | |
| | DTPNNQVFLKI | 120) | | 198) | | ADTA | | | | |
| | ASWTADTAT | | | | | TYYC | | | | |
| | YYCARIGGND | | | | | (SEQ | | | | |
| | GYYWYFDVW | | | | | ID NO: | | | | |
| | GAGTSLTVSS | | | | | 523) | | | | |
| | (SEQ ID NO: | | | | | | | | | |
| | 446) | | | | | | | | | |
| AHH | QIQLQQSGAEL | QIQLQ | GYTFTS | MHW | INPSN | NYNE | ARDSS | CARDS | YGAYW | WGQG |
| 03783 | VKPGASVKLS | QSGAE | YW | VKQR | GRT | KFKS | GYGA | SGYGA | GQGTLV | TLVT |
| | CKASGYTFTS | LVKPG | (SEQ ID | PGQG | (SEQ ID | KATL | Y (SEQ | YW | TVSS | VSS |
| | YWMHWVKQR | ASVKL | NO: | LEWI | NO: | TVDK | ID NO: | (SEQ ID | (SEQ ID | (SEQ |
| | PGQGLEWIGEI | SCKAS | 157) | GE | 510) | SSSTA | 544) | NO: | NO: 591) | ID NO: |
| | NPSNGRTNYN | (SEQ | | (SEQ | | YMQL | | 571) | | 413) |
| | EKFKSKATLT | ID NO: | | ID NO: | | SSLTS | | | | |
| | VDKSSSTAYM | 472) | | 209) | | EDSA | | | | |
| | QLSSLTSEDSA | | | | | VYYC | | | | |
| | VYYCARDSSG | | | | | (SEQ | | | | |
| | YGAYWGQGT | | | | | ID NO: | | | | |
| | LVTVSS (SEQ | | | | | 289) | | | | |
| | ID NO: 447) | | | | | | | | | |
| AHH | QVQLQQSGAE | QVQL | GYTFTS | MHW | INPSTG | EYNQ | ARYDG | CARYD | YFDYWG | WGQG |
| 03784 | LAKPGASVKM | QQSG | YW | VKQR | YT | KFKD | YYYFD | GYYYF | QGTTLT | TTLT |
| | SCKASGYTFTS | AELA | (SEQ ID | PGQG | (SEQ ID | KATL | Y (SEQ | DYW | VSS | VSS |
| | YWMHWVKQR | KPGAS | NO: | LEWI | NO: | TADK | ID NO: | (SEQ ID | (SEQ ID | (SEQ |
| | PGQGLEWIGYI | VKMS | 157) | GY | 230) | SSSTA | 545) | NO: | NO: 592) | ID NO: |
| | NPSTGYTEYN | CKAS | | (SEQ | | YMQL | | 572) | | 425) |
| | QKFKDKATLT | (SEQ | | ID NO: | | SSLTS | | | | |
| | ADKSSSTAYM | ID NO: | | 190) | | EDSA | | | | |
| | QLSSLTSEDSA | 473) | | | | VYYC | | | | |
| | VYYCARYDGY | | | | | (SEQ | | | | |
| | YYFDYWGQG | | | | | ID NO: | | | | |
| | TTLTVSS (SEQ | | | | | 265) | | | | |
| | ID NO: 448) | | | | | | | | | |
| AHH | QVQLQQSGAE | QVQL | GYTFTS | INWV | IYPSDS | NYNQ | TSHYY | CTSHY | WFAYW | WGQG |
| 03785 | LVRPGASVKL | QQSG | YW | KQRP | YT | KFKD | GRAW | YGRA | GQGTLV | TLVT |
| | SCKASGYTFTS | AELVR | (SEQ ID | GQGL | (SEQ ID | KATL | FAY | WFAY | TVL | VL |
| | YWINWVKQRP | PGASV | NO: | EWIG | NO: | TVDK | (SEQ ID | W (SEQ | (SEQ ID | (SEQ |
| | GQGLEWIGNIY | KLSCK | 157) | N | 509) | SSSTA | NO: | ID NO: | NO: 593) | ID NO: |
| | PSDSYTNYNQ | AS | | (SEQ | | YMQL | 546) | 573) | | 598) |
| | KFKDKATLTV | (SEQ | | ID NO: | | SSPTS | | | | |
| | DKSSSTAYMQ | ID NO: | | 500) | | EDSA | | | | |
| | LSSPTSEDSAV | 113) | | | | VYYC | | | | |
| | YYCTSHYYGR | | | | | (SEQ | | | | |
| | AWFAYWGQG | | | | | ID NO: | | | | |
| | TLVTVL (SEQ | | | | | 521) | | | | |
| | ID NO: 449) | | | | | | | | | |
| AHH | QIQLQQPGAEL | QIQLQ | GYTFT | MYW | INPSN | NFNE | TKGGF | CTKGG | FFAYWG | WGQG |
| 03786 | VKPGASVKLS | QPGAE | RYY | VKQR | GGT | KFKS | YDFFA | FYDFF | QGTLVT | TLVT |
| | CKASGYTFTR | LVKPG | (SEQ ID | PGQG | (SEQ ID | KATL | Y (SEQ | AYW | VSA | VSA |
| | YYMYWVKQR | ASVKL | NO: | LEWI | NO: | TVDK | ID NO: | (SEQ ID | (SEQ ID | (SEQ |
| | PGQGLEWIGEI | SCKAS | 488) | GE | 231) | SSSTA | 547) | NO: | NO: 594) | ID NO: |
| | NPSNGGTNFN | (SEQ | | (SEQ | | YMQL | | 574) | | 421) |
| | EKFKSKATLT | ID NO: | | ID NO: | | NSLTS | | | | |
| | VDKSSSTAYM | 474) | | 191) | | DDSA | | | | |
| | QLNSLTSDDSA | | | | | VYYC | | | | |
| | VYYCTKGGFY | | | | | (SEQ | | | | |
| | DFFAYWGQGT | | | | | ID NO: | | | | |
| | LVTVSA (SEQ | | | | | 524) | | | | |
| | ID NO: 450) | | | | | | | | | |
| AHH | EVMLVESGGG | EVML | GFTFSS | MSWV | INSNG | YYPD | ASLAY | CASLA | AYWGQ | WGQG |
| 03787 | LVQPGGSLKLS | VESGG | YG | RQTP | GST | SVKG | (SEQ ID | YW | GTLVTV | TLVT |
| | CAASGFTFSSY | GLVQP | (SEQ ID | DKRL | (SEQ ID | RFTIS | NO: | (SEQ ID | SA (SEQ | VSA |
| | GMSWVRQTPD | GGSLK | NO: | ELVA | NO: | RDNA | 548) | NO: | ID NO: | (SEQ |
| | KRLELVATINS | LSCAA | 165) | T(SEQ | 240) | KNTL | | 575) | 394) | ID NO: |
| | NGGSTYYPDS | S (SEQ | | ID NO: | | YLQM | | | | 421) |
| | VKGRFTISRDN | ID NO: | | 200) | | SSLKS | | | | |
| | AKNTLYLQMS | 122) | | | | EDTA | | | | |
| | SLKSEDTAMY | | | | | MYYC | | | | |
| | YCASLAYWGQ | | | | | (SEQ | | | | |
| | GTLVTVSA | | | | | ID NO: | | | | |
| | (SEQ ID NO: | | | | | 274) | | | | |
| | 451) | | | | | | | | | |
| AHH | QIQLQQSGPDL | QIQLQ | GYTFT | INWM | IYPGS | KYNE | ARVYS | CARVY | FDVWGA | WGAG |
| 03788 | VKPGASVKISC | QSGPD | DYY | KQKP | GNT | KFKG | GFDV | SGFDV | GTTVTV | TTVT |
| | KASGYTFTDY | LVKPG | (SEQ ID | GQGL | (SEQ ID | KATL | (SEQ ID | W (SEQ | SS (SEQ | VSS |
| | YINWMKQKPG | ASVKI | NO: | EWIG | NO: | TVDT | NO: | ID NO: | ID NO: | (SEQ |
| | QGLEWIGWIY | SCKAS | 489) | W | 511) | SSSTA | 549) | 576) | 585) | ID NO: |
| | PGSGNTKYNE | (SEQ | | (SEQ | | YMQL | | | | 423) |
| | KFKGKATLTV | ID NO: | | ID NO: | | SSLTS | | | | |
| | DTSSSTAYMQ | 475) | | 501) | | EDTA | | | | |
| | LSSLTSEDTAV | | | | | VYFC | | | | |
| | YFCARVYSGF | | | | | (SEQ | | | | |
| | DVWGAGTTVT | | | | | ID NO: | | | | |
| | VSS (SEQ ID | | | | | 525) | | | | |
| | NO: 452) | | | | | | | | | |
| AHH | QIQLQQSGPEL | QIQLQ | GYTFTS | IHWV | IYPGD | KYNE | ARGDG | CARGD | WFAYW | WGQG |
| 03790 | VKPGASVKMS | QSGPE | YY | KQRP | GST | KFKG | YFAWF | GYFA | GQGTLV | TLVT |
| | CKAPGYTFTS | LVKPG | (SEQ ID | GQGL | (SEQ ID | KTTL | AY | WFAY | TVSA | VSA |
| | YYIHWVKQRP | ASVK | NO: | EWIG | NO: | TADK | (SEQ ID | W (SEQ | (SEQ ID | (SEQ |
| | GQGLEWIGWI | MSCK | 158) | W | 512) | SSSTA | NO: | ID NO: | NO: 392) | ID NO: |
| | YPGDGSTKYN | AP | | (SEQ | | YMLL | 550) | 577) | | 421) |
| | EKFKGKTTLT | (SEQ | | ID NO: | | SSLTS | | | | |
| | ADKSSSTAYM | ID NO: | | 502) | | EDSAI | | | | |
| | LLSSLTSEDSAI | 476) | | | | YFC | | | | |
| | YFCARGDGYF | | | | | (SEQ | | | | |
| | AWFAYWGQG | | | | | ID NO: | | | | |
| | TLVTVSA (SEQ | | | | | 526) | | | | |
| | ID NO: 453) | | | | | | | | | |
| AHH | QIQLQQSGAEL | QIQLQ | GYTFSS | IEWV | ILPGSG | KYNE | ARSAH | CARSA | WFAYW | WGQG |
| 03791 | MKPGASVKIS | QSGAE | YW | KQRP | ST | KFKG | RYDA | HRYDA | GQGTLV | TLVT |
| | CKATGYTFSS | LMKP | (SEQ ID | GHGL | (SEQ ID | KATF | WFAY | WFAY | TVL | VL |
| | YWIEWVKQRP | GASV | NO: | EWIG | NO: | TADT | (SEQ ID | W (SEQ | (SEQ ID | (SEQ |
| | GHGLEWIGEIL | KISCK | 179) | SEQ | 254) | SSNT | NO: | ID NO: | NO: 593) | ID NO: |
| | PGSGSTKYNE | AT | | ID NO: | | AYMQ | 551) | 578) | | 598) |
| | KFKGKATFTA | (SEQ | | 218) | | LSSLT | | | | |
| | DTSSNTAYMQ | ID NO: | | | | SEDS | | | | |
| | LSSLTSEDSAV | 144) | | | | AVYY | | | | |
| | YYCARSAHRY | | | | | C | | | | |
| | DAWFAYWGQ | | | | | (SEQ | | | | |
| | GTLVTVL (SEQ | | | | | ID NO: | | | | |
| | ID NO: 454) | | | | | 527) | | | | |
| AHH | QIQLQQPGAEL | QIQLQ | GYTFT | MYW | INPTN | NFNA | TRGM | CTRGM | WFAYW | WGQG |
| 03792 | VKPGASVKLS | QPGAE | NYY | VNQR | GGT | KFKN | AYRYD | AYRYD | GQGTPV | TPVT |
| | CKASGYTFTN | LVKPG | (SEQ ID | PGQG | (SEQ ID | KATL | GAGW | GAGW | TVSS | VSS |
| | YYMYWVNQR | ASVKL | NO: | LEWI | NO: | TVDK | FAY | FAYW | (SEQ ID | (SEQ |
| | PGQGLEWIGGI | SCKAS | 490) | GG | 513) | SSNT | (SEQ ID | (SEQ ID | NO: 595) | ID NO: |
| | NPTNGGTNFN | (SEQ | | (SEQ | | AYMQ | NO: | NO: | | 398) |
| | AKFKNKATLT | ID NO: | | ID NO: | | LSSLT | 552) | 579) | | |
| | VDKSSNTAYM | 474) | | 503) | | SEDS | | | | |
| | QLSSLTSEDSA | | | | | AVYY | | | | |
| | VYYCTRGMA | | | | | C | | | | |
| | YRYDGAGWF | | | | | (SEQ | | | | |
| | AYWGQGTPVT | | | | | ID NO: | | | | |
| | VSS (SEQ ID | | | | | 528) | | | | |
| | NO: 455) | | | | | | | | | |
| AHH | QVQLQQPGAE | QVQL | GYTFTS | MNW | IDPYD | HYNQ | ARGGR | CARGG | WFAYW | WGQG |
| 03793 | LVRPGASVKL | QQPG | YW | VKQR | SET | KFKD | GTWFA | RGTWF | GQGTLV | TLVT |
| | SCKASGYTFTS | AELVR | (SEQ ID | PEQG | (SEQ ID | KAILT | Y (SEQ | AYW | TVSA | VSA |
| | YWMNWVKQR | PGASV | NO: | LEWI | NO: | VDKS | ID NO: | (SEQ ID | (SEQ ID | (SEQ |
| | PEQGLEWIGRI | KLSCK | 157) | GR | 235) | SSTA | 553) | NO: | NO: 392) | ID NO: |
| | DPYDSETHYN | AS | | (SEQ | | YMQL | | 580) | | 421) |
| | QKFKDKAILT | (SEQ | | ID NO: | | SSLTS | | | | |
| | VDKSSSTAYM | ID NO: | | 195) | | EDSA | | | | |
| | QLSSLTSEDSA | 477) | | | | VYYC | | | | |
| | VYYCARGGRG | | | | | (SEQ | | | | |
| | TWFAYWGQG | | | | | ID NO: | | | | |
| | TLVTVSA (SEQ | | | | | 269) | | | | |
| | ID NO: 456) | | | | | | | | | |
| AHH | DVMLVESGGG | DVML | GFTFTD | MSWV | IRNKA | EYSA | ARGW | CARG | WFAYW | WGQG |
| 03794 | LVQPGGSLRLS | VESGG | YY | RQPP | NGYTT | SVKG | GNWF | WGNW | GQGTLV | TLVT |
| | CATSGFTFTDY | GLVQP | (SEQ ID | GKAL | (SEQ ID | RFTIS | AY | FAYW | TVSA | VSA |
| | YMSWVRQPPG | GGSLR | NO: | EWLG | NO: | RDNS | (SEQ ID | (SEQ ID | (SEQ ID | (SEQ |
| | KALEWLGFIR | LSCAT | 180) | F (SEQ | 255) | QSILY | NO: | NO: | NO: 392) | ID NO: |
| | NKANGYTTEY | S (SEQ | | ID NO: | | LQMN | 554) | 581) | | 421) |
| | SASVKGRFTIS | ID NO: | | 219) | | TLRA | | | | |
| | RDNSQSILYLQ | 478) | | | | EDSA | | | | |
| | MNTLRAEDSA | | | | | TYYC | | | | |
| | TYYCARGWG | | | | | (SEQ | | | | |
| | NWFAYWGQG | | | | | ID NO: | | | | |
| | TLVTVSA (SEQ | | | | | 292) | | | | |
| | ID NO: 457) | | | | | | | | | |
| AHH | EVMLVESGGG | EVML | GFTFSS | IHWV | ISGGG | SYAD | ARWR | CARW | YFDYWG | WGQG |
| 03795 | LVQPGGSRKL | VESGG | FG (SEQ | RQAP | GTI | TVKG | GGYFD | RGGYF | QGTSLT | TSLTV |
| | SCAASGFTFSS | GLVQP | ID NO: | EKGL | (SEQ ID | RFTIS | Y (SEQ | DYW | VSS | SS |
| | FGIHWVRQAP | GGSR | 491) | EWVAY | NO: | RDNP | ID NO: | (SEQ ID | (SEQ ID | (SEQ |
| | EKGLEWVAYI | KLSCA | | (SEQ | 514) | KNTL | 555) | NO: | NO: 405) | ID NO: |
| | SGGGGTISYAD | AS | | ID NO: | | FLQM | | 582) | | 424) |
| | TVKGRFTISRD | (SEQ | | 504) | | TSLRS | | | | |
| | NPKNTLFLQM | ID NO: | | | | EDTAI | | | | |
| | TSLRSEDTAIY | 479) | | | | YYC | | | | |
| | YCARWRGGYF | | | | | (SEQ | | | | |
| | DYWGQGTSLT | | | | | ID NO: | | | | |
| | VSS (SEQ ID | | | | | 529) | | | | |
| | NO: 458) | | | | | | | | | |
| AHH | QIQLQQSGAEL | QIQLQ | GYTFTS | INWV | IYPSDS | NYNQ | TRTGG | CTRTG | WFAYW | WGQG |
| 03797 | VRPGASVKLS | QSGAE | YW | KQRP | YT | KFKD | STMTP | GSTMT | GQGTLV | TLVT |
| | CKASGYTFTS | LVRPG | (SEQ ID | GQGL | (SEQ ID | KATL | WFAY | PWFAY | TVSS | VSS |
| | YWINWVKQRP | ASVKL | NO: | EWIG | NO: | TVDK | (SEQ ID | W (SEQ | (SEQ ID | (SEQ |
| | GQGLEWIGNIY | SCKAS | 157) | N | 509) | SSSTA | NO: | ID NO: | NO: 588) | ID NO: |
| | PSDSYTNYNQ | (SEQ | | (SEQ | | YMQL | 556) | 583) | | 413) |
| | KFKDKATLTV | ID NO: | | ID NO: | | SSPTS | | | | |
| | DKSSSTAYMQ | 480) | | 500) | | EDSA | | | | |
| | LSSPTSEDSAV | | | | | VYYC | | | | |
| | YYCTRTGGST | | | | | (SEQ | | | | |
| | MTPWFAYWG | | | | | ID NO: | | | | |
| | QGTLVTVSS | | | | | 521) | | | | |
| | (SEQ ID NO: | | | | | | | | | |
| | 459) | | | | | | | | | |
| AHH | QVTLKESGPGI | QVTL | GFSLST | VSWI | IYWDD | RYNP | ARRAG | CARRA | FPYWGQ | WGQG |
| 03799 | LQPSQTLSLTC | KESGP | SGMG | RQPS | DK | SLKSR | DYGNP | GDYG | GTLVTV | TLVT |
| | SFSGFSLSTSG | GILQP | (SEQ ID | GKGL | (SEQ ID | LTISK | FPY | NPFPY | SA (SEQ | VSA |
| | MGVSWIRQPS | SQTLS | NO: | EWLA | NO: | DTSR | (SEQ ID | W (SEQ | ID NO: | (SEQ |
| | GKGLEWLAHI | LTCSF | 492) | H | 515) | NQVF | NO: | ID NO: | 596) | ID NO: |
| | YWDDDKRYN | S (SEQ | | (SEQ | | LKITS | 557) | 584) | | 421) |
| | PSLKSRLTISK | ID NO: | | ID NO: | | VDTT | | | | |
| | DTSRNQVFLKI | 481) | | 505) | | DTAT | | | | |
| | TSVDTTDTAT | | | | | YYC | | | | |
| | YYCARRAGDY | | | | | (SEQ | | | | |
| | GNPFPYWGQG | | | | | ID NO: | | | | |
| | TLVTVSA (SEQ | | | | | 530) | | | | |
| | ID NO: 460) | | | | | | | | | |

TABLE 2C
Exemplary Clones-Heavy Chain Sequences

| | | | | | | | | JUNC- |
|---|---|---|---|---|---|---|---|---|
| ID | V-D-J-REGION | H-FR1 | CDRH1 | H-FR2 | CDRH2 | H-FR3 | CDRH3 | TION |
| >AH04501- | QVQLQQPGAELVR | QVQLQ | GYTFT | WIGW | DIHPG | KATLTA | SRNFAY | WGQGT |
| VH | PGTSVKMSCKAAG | QPGAEL | NY | VKQR | GDYSN | DTSSST | (SEQ ID | LVTVSA |
| | YTFTNYWIGWVKQ | VRPGTS | (SEQ | PGHGL | YNEKF | AYMNLS | NO: | (SEQ ID |
| | RPGHGLEWIGDIHP | VKMSC | ID NO: | EWIG | KG | SLTSEDS | 1379) | NO: 421) |
| | GGDYSNYNEKFKG | KAA | 990) | (SEQ | (SEQ | AIYYCT | | |
| | KATLTADTSSSTAY | (SEQ ID | | ID NO: | ID NO: | (SEQ ID | | |
| | MNLSSLTSEDSAIY | NO: | | 1458) | 692) | NO: | | |
| | YCTSRNFAYWGQG | 1315) | | | | 1044) | | |
| | TLVTVSA (SEQ ID | | | | | | | |
| | NO: 1318) | | | | | | | |
| >AH04515- | QVQLQQPGAELVR | QVQLQ | GYTFT | WIGW | DIHPG | KATLTA | SRNFAY | WGQGT |
| VH | PGTSVKMSCKAAG | QPGAEL | NY | VKQR | GDYSN | DTSSST | (SEQ ID | LVTVSA |
| | YTFTNYWIGWVKQ | VRPGTS | (SEQ | PGHGL | YNEKF | AYMNLS | NO: | (SEQ ID |
| | RPGHGLEWIGDIHP | VKMSC | ID NO: | EWIG | KG | SLTSEDS | 1379) | NO: 421) |
| | GGDYSNYNEKFKG | KAA | 990) | (SEQ | (SEQ | AIYYCT | | |
| | KATLTADTSSSTAY | (SEQ ID | | ID NO: | ID NO: | (SEQ ID | | |
| | MNLSSLTSEDSAIY | NO: | | 1458) | 692) | NO: | | |
| | YCTSRNFAYWGQG | 1315) | | | | 1044) | | |
| | TLVTVSA (SEQ ID | | | | | | | |
| | NO: 1318) | | | | | | | |
| >AH04502- | QIQLQQSGAELVRP | QIQLQQ | GYTFT | WIGW | DIHPG | KATLTA | SRNFAY | WGQGT |
| VH | GTSVKMSCKAAGY | SGAELV | NY | VKQR | GGYT | DTSSST | (SEQ ID | LVTVSA |
| | TFTNYWIGWVKQR | RPGTSV | (SEQ | PGHGL | NYNE | ADMQLS | NO: | (SEQ ID |
| | PGHGLEWIGDIHPG | KMSCK | ID NO: | EWIG | KFKG | SLTSEDS | 1379) | NO: 421) |
| | GGYTNYNEKFKGK | AA (SEQ | 990) | (SEQ | (SEQ | AIYYCT | | |
| | ATLTADTSSSTAD | ID NO: | | ID NO: | ID NO: | (SEQ ID | | |
| | MQLSSLTSEDSAIY | 1228) | | 1458) | 696) | NO: | | |
| | YCTSRNFAYWGQG | | | | | 1043) | | |
| | TLVTVSA (SEQ ID | | | | | | | |
| | NO: 1234) | | | | | | | |
| >AH04512- | QIQLQQSGAELVRP | QIQLQQ | GYTFT | WIGW | DIHPG | KATLTA | SRNFAY | WGQGT |
| VH | GTSVKMSCKAAGY | SGAELV | NY | VKQR | GGYT | DTSSST | (SEQ ID | LVTVSA |
| | TFTNYWIGWVKQR | RPGTSV | (SEQ | PGHGL | NYNE | AYMQLS | NO: | (SEQ ID |
| | PGHGLEWIGDIHPG | KMSCK | ID NO: | EWIG | KFKG | SLTSEDS | 1379) | NO: 421) |
| | GGYTNYNEKFKGK | AA (SEQ | 990) | (SEQ | (SEQ | AIYYCT | | |
| | ATLTADTSSSTAY | ID NO: | | ID NO: | ID NO: | (SEQ ID | | |
| | MQLSSLTSEDSAIY | 1228) | | 1458) | 696) | NO: | | |
| | YCTSRNFAYWGQG | | | | | 1045) | | |
| | TLVTVSA (SEQ ID | | | | | | | |
| | NO: 1235) | | | | | | | |
| >AH04522- | QIQLQQSGAELVRP | QIQLQQ | GYTFT | WIGW | DIHPG | KATLTA | SRNFAY | WGQGT |
| VH | GTSVKMSCKAAGY | SGAELV | NY | VKQR | GGYT | DTSSST | (SEQ ID | LVTVSA |
| | TFTNYWIGWVKQR | RPGTSV | (SEQ | PGHGL | NYNE | AYMQLS | NO: | (SEQ ID |
| | PGHGLEWIGDIHPG | KMSCK | ID NO: | EWIG | KFKG | SLTSEDS | 1379) | NO: 421) |
| | GGYTNYNEKFKGK | AA (SEQ | 990) | (SEQ | (SEQ | AIYYCT | | |
| | ATLTADTSSSTAY | ID NO: | | ID NO: | ID NO: | (SEQ ID | | |
| | MQLSSLTSEDSAIY | 1228) | | 1458) | 696) | NO: | | |
| | YCTSRNFAYWGQG | | | | | 1045) | | |
| | TLVTVSA (SEQ ID | | | | | | | |
| | NO: 1235) | | | | | | | |
| >AH04513- | QIQLQQSGAELVRP | QIQLQQ | GYTFT | WIGW | DIHPG | KATLTA | SRNFAY | WGQGT |
| VH | GTSVKMSCKAAGY | SGAELV | NY | VKQR | GGYT | DTSSST | (SEQ ID | PVTVSS |
| | TFTNYWIGWVKQR | RPGTSV | (SEQ | PGHGL | NYNE | AYMQLS | NO: | (SEQ ID |
| | PGHGLEWIGDIHPG | KMSCK | ID NO: | EWIG | KFKG | SLTSEDS | 1379) | NO: 398) |
| | GGYTNYNEKFKGK | AA (SEQ | 990) | (SEQ | (SEQ | AIYYCT | | |
| | ATLTADTSSSTAY | ID NO: | | ID NO: | ID NO: | (SEQ ID | | |
| | MQLSSLTSEDSAIY | 1228) | | 1458) | 696) | NO: | | |
| | YCTSRNFAYWGQG | | | | | 1045) | | |
| | TPVTVSS (SEQ ID | | | | | | | |
| | NO: 1236) | | | | | | | |
| >AH04523- | QIQLQQSGAELVRP | QIQLQQ | GYTFT | WIGW | DIHPG | KATFTA | SRNFAK | WGQGT |
| VH | GTSVKMSCKAAGY | SGAELV | NY | VKQR | GSYTN | DTSSSTT | (SEQ ID | PVTVSS |
| | TFTNYWIGWVKQR | RPGTSV | (SEQ | PGHGL | YNENF | YMQLSS | NO: | (SEQ ID |
| | PGHGLEWIGDIHPG | KMSCK | ID NO: | EWIG | KG | LTSEDS | 1377) | NO: 398) |
| | GSYTNYNENFKGK | AA (SEQ | 990) | (SEQ | (SEQ | AIYFCT | | |
| | ATFTADTSSSTTYM | ID NO: | | ID NO: | ID NO: | (SEQ ID | | |
| | QLSSLTSEDSAIYFC | 1228) | | 1458) | 697) | NO: | | |
| | TSRNFAKWGQGTP | | | | | 1041) | | |
| | VTVSS (SEQ ID NO: | | | | | | | |
| | 1237) | | | | | | | |
| >AH04503- | QVQLQQPGAELVR | QVQLQ | GYTFS | WIGW | DIHPG | KATLTA | SRNFAN | WGQGT |
| VH | PGTSVKMSCKAAG | QPGAEL | NY | VKQR | GGYIN | DTSSST | (SEQ ID | LVTVSA |
| | YTFSNYWIGWVKQ | VRPGTS | (SEQ | PGHGL | YNEKF | AYMQLS | NO: | (SEQ ID |
| | RPGHGLEWIGDIHP | VKMSC | ID NO: | EWIG | TG | SLTSEDS | 1378) | NO: 421) |
| | GGGYINYNEKFTG | KAA | 985) | (SEQ | (SEQ | AIYYCV | | |
| | KATLTADTSSSTAY | (SEQ ID | | ID NO: | ID NO: | (SEQ ID | | |
| | MQLSSLTSEDSAIY | NO: | | 1458) | 695) | NO: | | |
| | YCVSRNFANWGQG | 1315) | | | | 1046) | | |
| | TLVTVSA (SEQ ID | | | | | | | |
| | NO: 1316) | | | | | | | |
| >AH04529- | QVQLQQPGAELVR | QVQLQ | GYTFS | WIGW | DIHPG | KATLTA | SRNFAN | WGQGT |
| VH | PGTSVKMSCKAAG | QPGAEL | NY | VKQR | GGYIN | DTSSST | (SEQ ID | PVTVSS |
| | YTFSNYWIGWVKQ | VRPGTS | (SEQ | PGHGL | YNEKF | AYMQLS | NO: | (SEQ ID |
| | RPGHGLEWIGDIHP | VKMSC | ID NO: | EWIG | TG | SLTSEDS | 1378) | NO: 398) |
| | GGGYINYNEKFTG | KAA | 985) | (SEQ | (SEQ | AIYYCV | | |
| | KATLTADTSSSTAY | (SEQ ID | | ID NO: | ID NO: | (SEQ ID | | |
| | MQLSSLTSEDSAIY | NO: | | 1458) | 695) | NO: | | |
| | YCVSRNFANWGQG | 1315) | | | | 1046) | | |
| | TPVTVSS (SEQ ID | | | | | | | |
| | NO: 1317) | | | | | | | |
| >AH04510- | QVQLQQSGAELVR | QVQLQ | GYTFS | WIGW | DIHPG | KATLTA | SRNFAN | WGQGT |
| VH | PGTSVKMSCKAAG | QSGAEL | NY | VKQR | GGYIN | DTSSST | (SEQ ID | LVTVSA |
| | YTFSNYWIGWVKQ | VRPGTS | (SEQ | PGHGL | YNEKF | AYMQLS | NO: | (SEQ ID |
| | RPGHGLEWIGDIHP | VKMSC | ID NO: | EWIG | TG | SLTSEDS | 1378) | NO: 421) |
| | GGGYINYNEKFTG | KAA | 985) | (SEQ | (SEQ | AIYYCV | | |
| | KATLTADTSSSTAY | (SEQ ID | | ID NO: | ID NO: | (SEQ ID | | |
| | MQLSSLTSEDSAIY | NO: | | 1458) | 695) | NO: | | |
| | YCVSRNFANWGQG | 1326) | | | | 1046) | | |
| | TLVTVSA (SEQ ID | | | | | | | |
| | NO: 1327) | | | | | | | |
| >AH04528- | QVQLQQSGAELVR | QVQLQ | GYTFS | WIGW | DIHPG | KATLTA | SRNFAN | WGQGT |
| VH | PGTSVKMSCKAAG | QSGAEL | NY | VKQR | GGYIN | DTSSST | (SEQ ID | LVTVSA |
| | YTFSNYWIGWVKQ | VRPGTS | (SEQ | PGRGL | YNEKF | AYMQLS | NO: | (SEQ ID |
| | RPGRGLEWIGDIHP | VKMSC | ID NO: | EWIG | TG | SLTSGD | 1378) | NO: 421) |
| | GGGYINYNEKFTG | KAA | 985) | (SEQ | (SEQ | SAIYYC | | |
| | KATLTADTSSSTAY | (SEQ ID | | ID NO: | ID NO: | V (SEQ | | |
| | MQLSSLTSGDSAIY | NO: | | 1459) | 695) | ID NO: | | |
| | YCVSRNFANWGQG | 1326) | | | | 1048) | | |
| | TLVTVSA (SEQ ID | | | | | | | |
| | NO: 1328) | | | | | | | |
| >AH04511- | QIQLQQSGAELVRP | QIQLQQ | GYTFS | WIGW | DIHPG | KATLTA | SRNFAN | WGQGT |
| VH | GTSVKMSCKAAGY | SGAELV | NY | VKQR | GGYIN | DTSSST | (SEQ ID | LVTVSA |
| | TFSNYWIGWVKQR | RPGTSV | (SEQ | PGHGL | YNEKF | AYMQLS | NO: | (SEQ ID |
| | PGHGLEWIGDIHPG | KMSCK | ID NO: | EWIG | TG | SLTSEDS | 1378) | NO: 421) |
| | GGYINYNEKFTGK | AA (SEQ | 985) | (SEQ | (SEQ | AIYYCV | | |
| | ATLTADTSSSTAY | ID NO: | | ID NO: | ID NO: | (SEQ ID | | |
| | MQLSSLTSEDSAIY | 1228) | | 1458) | 695) | NO: | | |
| | YCVSRNFANWGQG | | | | | 1046) | | |
| | TLVTVSA (SEQ ID | | | | | | | |
| | NO: 1229) | | | | | | | |
| >AH04504- | QIQLQQSGAELVRP | QIQLQQ | GYTFT | WIGW | DIHPG | KATLTA | SRNFAN | WGQGT |
| VH | GTSVKMSCKAAGY | SGAELV | KY | VKQR | GGYIN | DTSSST | (SEQ ID | LVTVSA |
| | TFTKYWIGWVKQR | RPGTSV | (SEQ | SGHGL | YNEKF | AYMQLS | NO: | (SEQ ID |
| | SGHGLEWIGDIHPG | KMSCK | ID NO: | EWIG | TG | SLTSEDS | 1378) | NO: 421) |
| | GGYINYNEKFTGK | AA (SEQ | 989) | (SEQ | (SEQ | AIYYCV | | |
| | ATLTADTSSSTAY | ID NO: | | ID NO: | ID NO: | (SEQ ID | | |
| | MQLSSLTSEDSAIY | 1228) | | 1460) | 695) | NO: | | |
| | YCVSRNFANWGQG | | | | | 1046) | | |
| | TLVTVSA (SEQ ID | | | | | | | |
| | NO: 1230) | | | | | | | |
| >AH04520- | QIQLQQPGAELVRP | QIQLQQ | GYTFT | WIGW | DIHPG | KATLTA | SRNFAN | WGQGT |
| VH | GTSVKMSCKAAGY | PGAELV | KY | VKQR | GGYIN | GTSSST | (SEQ ID | LVTVSA |
| | TFTKYWIGWVKQR | RPGTSV | (SEQ | PGHGL | YNEKF | AYMQLS | NO: | (SEQ ID |
| | PGHGLEWIGDIHPG | KMSCK | ID NO: | EWIG | TG | SLTSEDS | 1378) | NO: 421) |
| | GGYINYNEKFTGK | AA (SEQ | 989) | (SEQ | (SEQ | AIYYCV | | |
| | ATLTAGTSSSTAY | ID NO: | | ID NO: | ID NO: | (SEQ ID | | |
| | MQLSSLTSEDSAIY | 1221) | | 1458) | 695) | NO: | | |
| | YCVSRNFANWGQG | | | | | 1050) | | |
| | TLVTVSA (SEQ ID | | | | | | | |
| | NO: 1222) | | | | | | | |
| >AH04507- | QVQLQQSGAELVR | QVQLQ | GYTFT | WIGW | DIHPG | KATLTA | SRNFAK | WGQGT |
| VH | PGTSVKMSCKAAG | QSGAEL | NY | VKQR | GGYID | DTSSST | (SEQ ID | LVTVSA |
| | YTFTNYWIGWVKQ | VRPGTS | (SEQ | PGHGL | YNEKF | AYMQLS | NO: | (SEQ ID |
| | RPGHGLEWIGDIHP | VKMSC | ID NO: | EWIG | TG | SLTSEDS | 1377) | NO: 421) |
| | GGGYIDYNEKFTG | KAA | 990) | (SEQ | (SEQ | AIYYCV | | |
| | KATLTADTSSSTAY | (SEQ ID | | ID NO: | ID NO: | (SEQ ID | | |
| | MQLSSLTSEDSAIY | NO: | | 1458) | 694) | NO: | | |
| | YCVSRNFAKWGQG | 1326) | | | | 1046) | | |
| | TLVTVSA (SEQ ID | | | | | | | |
| | NO: 1334) | | | | | | | |
| >AH04527- | QVQLQQSGAELVR | QVQLQ | GYTFT | WIGW | DIHPG | KATLTA | SRNFAK | WGQGT |
| VH | PGTSVKMSCKAAG | QSGAEL | NY | VKQR | GGYID | DTSSST | (SEQ ID | LVTVSA |
| | YTFTNYWIGWVKQ | VRPGTS | (SEQ | PGHGL | YNEKF | AYMQLS | NO: | (SEQ ID |
| | RPGHGLEWIGDIHP | VKMSC | ID NO: | EWIG | TG | SLTSEDS | 1377) | NO: 421) |
| | GGGYIDYNEKFTG | KAA | 990) | (SEQ | (SEQ | AIYYCV | | |
| | KATLTADTSSSTAY | (SEQ ID | | ID NO: | ID NO: | (SEQ ID | | |
| | MQLSSLTSEDSAIY | NO: | | 1458) | 694) | NO: | | |
| | YCVSRNFAKWGQG | 1326) | | | | 1046) | | |
| | TLVTVSA (SEQ ID | | | | | | | |
| | NO: 1334) | | | | | | | |
| >AH04505- | DVQLQQSGAELVR | DVQLQ | GYTFT | WIGW | DIHPG | KATLTA | SRNFAY | WGQGT |
| VH | PGTSVKMSCKAAG | QSGAEL | NY | VKQR | GGYT | DTSSST | (SEQ ID | LVTVSA |
| | YTFTNYWIGWVKQ | VRPGTS | (SEQ | PGHGL | NYNE | AYMQLS | NO: | (SEQ ID |
| | RPGHGLEWIGDIHP | VKMSC | ID NO: | EWIG | KFKG | SLTSEGS | 1379) | NO: 421) |
| | GGGYTNYNEKFKG | KAA | 990) | (SEQ | (SEQ | AIYYCT | | |
| | KATLTADTSSSTAY | (SEQ ID | | ID NO: | ID NO: | (SEQ ID | | |
| | MQLSSLTSEGSAIY | NO: 867) | | 1458) | 696) | NO: | | |
| | YCTSRNFAYWGQG | | | | | 1047) | | |
| | TLVTVSA (SEQ ID | | | | | | | |
| | NO: 869) | | | | | | | |
| >AH04516- | QVQLQQSGAELVR | QVQLQ | GYTFT | WIGW | DIHPG | KATLTA | SRNFAY | WGQGT |
| VH | PGTSVKMSCKAAG | QSGAEL | NY | VKQR | GGYT | DTSSST | (SEQ ID | LVTVSA |
| | YTFTNYWIGWVKQ | VRPGTS | (SEQ | PGHGL | NYNE | AYMQLS | NO: | (SEQ ID |
| | RPGHGLEWIGDIHP | VKMSC | ID NO: | EWIG | KFKG | SLTSEDS | 1379) | NO: 421) |
| | GGGYTNYNEKFKG | KAA | 990) | (SEQ | (SEQ | AIYYCT | | |
| | KATLTADTSSSTAY | (SEQ ID | | ID NO: | ID NO: | (SEQ ID | | |
| | MQLSSLTSEDSAIY | NO: | | 1458) | 696) | NO: | | |
| | YCTSRNFAYWGQG | 1326) | | | | 1045) | | |
| | TLVTVSA (SEQ ID | | | | | | | |
| | NO: 1335) | | | | | | | |
| >AH04525- | QVQLQQSGAELVR | QVQLQ | GYTFT | WIGWI | DIHPG | KATLTA | GRNFAY | WGQGT |
| VH | PGTSVKMSCKAAG | QSGAEL | NY | KQRP | GDYT | DTFSST | (SEQ ID | LVTVSS |
| | YTFTNYWIGWIKQ | VRPGTS | (SEQ | GHGL | NYNE | AYMQLS | NO: 982) | (SEQ ID |
| | RPGHGLEWIGDIHP | VKMSC | ID NO: | EWIG | KFKG | SLTSEDS | | NO: 413) |
| | GGDYTNYNEKFKG | KAA | 990) | (SEQ | (SEQ | AIYYCT | | |
| | KATLTADTFSSTAY | (SEQ ID | | ID NO: | ID NO: | (SEQ ID | | |
| | MQLSSLTSEDSAIY | NO: | | 1456) | 693) | NO: | | |
| | YCTGRNFAYWGQ | 1326) | | | | 1042) | | |
| | GTLVTVSS (SEQ ID | | | | | | | |
| | NO: 1329) | | | | | | | |
| >AH04509- | QIQLQQSGAELVRP | QIQLQQ | GYTFT | WIGW | DFYPG | KATLTA | SRNFAY | WGQGT |
| VH | GTSVKMSCKAAGY | SGAELV | NY | VKQR | GDYIN | DTSSST | (SEQ ID | PVTVSS |
| | TFTNYWIGWVKQR | RPGTSV | (SEQ | PGHGL | YNEKF | AYMQLS | NO: | (SEQ ID |
| | PGHGLEWIGDFYP | KMSCK | ID NO: | EWIG | KG | SLTSEDS | 1379) | NO: 398) |
| | GGDYINYNEKFKG | AA (SEQ | 990) | (SEQ | (SEQ | AIYYCT | | |
| | KATLTADTSSSTAY | ID NO: | | ID NO: | ID NO: | (SEQ ID | | |
| | MQLSSLTSEDSAIY | 1228) | | 1458) | 691) | NO: | | |
| | YCTSRNFAYWGQG | | | | | 1045) | | |
| | TPVTVSS (SEQ ID | | | | | | | |
| | NO: 1231) | | | | | | | |
| >AH04521- | QVQLQQSGAELVR | QVQLQ | GYTFT | WIGW | DFYPG | KATLTA | SRNFAY | WGQGT |
| VH | PGTSVKMSCKAAG | QSGAEL | NY | VKQR | GDYIN | DTSSST | (SEQ ID | LVTVSA |
| | YTFTNYWIGWVKQ | VRPGTS | (SEQ | PGHGL | YNEKF | AYMQLS | NO: | (SEQ ID |
| | RPGHGLEWIGDFYP | VKMSC | ID NO: | EWIG | KG | SLTSEDS | 1379) | NO: 421) |
| | GGDYINYNEKFKG | KAA | 990) | (SEQ | (SEQ | AIYYCT | | |
| | KATLTADTSSSTAY | (SEQ ID | | ID NO: | ID NO: | (SEQ ID | | |
| | MQLSSLTSEDSAIY | NO: | | 1458) | 691) | NO: | | |
| | YCTSRNFAYWGQG | 1326) | | | | 1045) | | |
| | TLVTVSA (SEQ ID | | | | | | | |
| | NO: 1331) | | | | | | | |
| >AH04526- | QVQLQQSGAELVR | QVQLQ | GYTFT | WIGW | DIHPG | KATLTA | SRNFAY | WGQGT |
| VH | PGTSVKMSCKAAG | QSGAEL | NY | VKQR | GDYSN | DTSSST | (SEQ ID | PVTVSS |
| | YTFTNYWIGWVKQ | VRPGTS | (SEQ | PGHGL | YNEKF | AYMNLS | NO: | (SEQ ID |
| | RPGHGLEWIGDIHP | VKMSC | ID NO: | EWIG | KG | SLTSEDS | 1379) | NO: 398) |
| | GGDYSNYNEKFKG | KAA | 990) | (SEQ | (SEQ | AIYYCT | | |
| | KATLTADTSSSTAY | (SEQ ID | | ID NO: | ID NO: | (SEQ ID | | |
| | MNLSSLTSEDSAIY | NO: | | 1458) | 692) | NO: | | |
| | YCTSRNFAYWGQG | 1326) | | | | 1044) | | |
| | TPVTVSS (SEQ ID | | | | | | | |
| | NO: 1332) | | | | | | | |
| >AH04514- | QIQLQQSGAELVRP | QIQLQQ | GYTFT | WIGW | DIHPG | KATLTA | SRNFAY | WGQGT |
| VH | GTSVKMSCKAAGY | SGAELV | NY | VKQR | GDYSN | DTSSST | (SEQ ID | LVTVSS |
| | TFTNYWIGWVKQR | RPGTSV | (SEQ | PGHGL | YNEKF | AYMNLS | NO: | (SEQ ID |
| | PGHGLEWIGDIHPG | KMSCK | ID NO: | EWIG | KG | SLTSEDS | 1379) | NO: 413) |
| | GDYSNYNEKFKGK | AA (SEQ | 990) | (SEQ | (SEQ | AIYYCT | | |
| | ATLTADTSSSTAY | ID NO: | | ID NO: | ID NO: | (SEQ ID | | |
| | MNLSSLTSEDSAIY | 1228) | | 1458) | 692) | NO: | | |
| | YCTSRNFAYWGQG | | | | | 1044) | | |
| | TLVTVSS (SEQ ID | | | | | | | |
| | NO: 1232) | | | | | | | |
| >AH04524- | QIQLQQSGAELVRP | QIQLQQ | GYTFT | WIGW | DIHPG | KATLTA | SRNFAY | WGQGT |
| VH | GTSVKMSCKAAGY | SGAELV | NY | VKQR | GDYSN | DTSSST | (SEQ ID | LVTVSA |
| | TFTNYWIGWVKQR | RPGTSV | (SEQ | PGHGL | YNEKF | AYMSLS | NO: | (SEQ ID |
| | PGHGLEWIGDIHPG | KMSCK | ID NO: | EWIG | KG | SLTSEDS | 1379) | NO: 421) |
| | GDYSNYNEKFKGK | AA (SEQ | 990) | (SEQ | (SEQ | AIYYCT | | |
| | ATLTADTSSSTAY | ID NO: | | ID NO: | ID NO: | (SEQ ID | | |
| | MSLSSLTSEDSAIY | 1228) | | 1458) | 692) | NO: | | |
| | YCTSRNFAYWGQG | | | | | 1049) | | |
| | TLVTVSA (SEQ ID | | | | | | | |
| | NO: 1233) | | | | | | | |
| >AH04530- | QIQLQQSGAELVRP | QIQLQQ | GYTST | WIGW | DIHPG | KATLTA | SRNFAY | WGQGT |
| VH | GTSVKMSCKAAGY | SGAELV | NY | VKQR | GDYSN | DTSSST | (SEQ ID | LVTVSA |
| | TSTNYWIGWVKQR | RPGTSV | (SEQ | PGHGL | YNEKF | AYMNLS | NO: | (SEQ ID |
| | PGHGLEWIGDIHPG | KMSCK | ID NO: | EWIG | KG | SLTSEDS | 1379) | NO: 421) |
| | GDYSNYNEKFKGK | AA (SEQ | 996) | (SEQ | (SEQ | AIYYCT | | |
| | ATLTADTSSSTAY | ID NO: | | ID NO: | ID NO: | (SEQ ID | | |
| | MNLSSLTSEDSAIY | 1228) | | 1458) | 692) | NO: | | |
| | YCTSRNFAYWGQG | | | | | 1044) | | |
| | TLVTVSA (SEQ ID | | | | | | | |
| | NO: 1238) | | | | | | | |
| >AH04517- | DVQLQQSGAELVR | DVQLQ | GYTFT | WIGW | DIHPG | KATLTA | SRNFAY | WGQGT |
| VH | PGTSVKMSCKAAG | QSGAEL | NY | VKQR | GDYSN | DTSSST | (SEQ ID | LVTVSA |
| | YTFTNYWIGWVKQ | VRPGTS | (SEQ | PGHGL | YNEKF | AYMNLS | NO: | (SEQ ID |
| | RPGHGLEWIGDIHP | VKMSC | ID NO: | EWIG | KG | SLTSEDS | 1379) | NO: 421) |
| | GGDYSNYNEKFKG | KAA | 990) | (SEQ | (SEQ | AIYYCT | | |
| | KATLTADTSSSTAY | (SEQ ID | | ID NO: | ID NO: | (SEQ ID | | |
| | MNLSSLTSEDSAIY | NO: 867) | | 1458) | 692) | NO: | | |
| | YCTSRNFAYWGQG | | | | | 1044) | | |
| | TLVTVSA (SEQ ID | | | | | | | |
| | NO: 868) | | | | | | | |
| >AH04506- | QVQLQQSGAELVR | QVQLQ | GYTFT | WIGW | DIHPG | KATLTA | SRNFAY | WGQGT |
| VH | PGTSVKMSCKAAG | QSGAEL | NY | VKQR | GDYSN | DTSSST | (SEQ ID | LVTVSA |
| | YTFTNYWIGWVKQ | VRPGTS | (SEQ | PGHGL | YNEKF | AYMNLS | NO: | (SEQ ID |
| | RPGHGLEWIGDIHP | VKMSC | ID NO: | EWIG | KG | SLTSEDS | 1379) | NO: 421) |
| | GGDYSNYNEKFKG | KAA | 990) | (SEQ | (SEQ | AIYYCT | | |
| | KATLTADTSSSTAY | (SEQ ID | | ID NO: | ID NO: | (SEQ ID | | |
| | MNLSSLTSEDSAIY | NO: | | 1458) | 692) | NO: | | |
| | YCTSRNFAYWGQG | 1326) | | | | 1044) | | |
| | TLVTVSA (SEQ ID | | | | | | | |
| | NO: 1333) | | | | | | | |
| >AH04508- | QVQLQQSGAELVR | QVQLQ | GYTFT | WIGW | DIHPG | KATLTA | SRNFAY | WGQGT |
| VH | PGTSVKMSCKAAG | QSGAEL | NY | VKQR | GDYSN | DTSSST | (SEQ ID | LVTVSA |
| | YTFTNYWIGWVKQ | VRPGTS | (SEQ | PGHGL | YNEKF | AYMNLS | NO: | (SEQ ID |
| | RPGHGLEWIGDIHP | VKMSC | ID NO: | EWIG | KG | SLTSEDS | 1379) | NO: 421) |
| | GGDYSNYNEKFKG | KAA | 990) | (SEQ | (SEQ | AIYYCT | | |
| | KATLTADTSSSTAY | (SEQ ID | | ID NO: | ID NO: | (SEQ ID | | |
| | MNLSSLTSEDSAIY | NO: | | 1458) | 692) | NO: | | |
| | YCTSRNFAYWGQG | 1326) | | | | 1044) | | |
| | TLVTVSA (SEQ ID | | | | | | | |
| | NO: 1333) | | | | | | | |
| >AH04518- | QVQLQQSGAELVR | QVQLQ | GYTFT | WIGW | DIHPG | KATLTA | SRNFAY | WGQGT |
| VH | PGTSVKMSCKAAG | QSGAEL | NY | VKQR | GDYSN | DTSSST | (SEQ ID | LVTVSA |
| | YTFTNYWIGWVKQ | VRPGTS | (SEQ | PGHDL | YNEKF | AYMNLS | NO: | (SEQ ID |
| | RPGHDLEWIGDIHP | VKMSC | ID NO: | EWIG | KG | SLTSEDS | 1379) | NO: 421) |
| | GGDYSNYNEKFKG | KAA | 990) | (SEQ | (SEQ | AIYYCT | | |
| | KATLTADTSSSTAY | (SEQ ID | | ID NO: | ID NO: | (SEQ ID | | |
| | MNLSSLTSEDSAIY | NO: | | 1457) | 692) | NO: | | |
| | YCTSRNFAYWGQG | 1326) | | | | 1044) | | |
| | TLVTVSA (SEQ ID | | | | | | | |
| | NO: 1330) | | | | | | | |

TABLE 3A
Exemplary Clones-Light Chain Sequences

| | | | | | | | | JUNC- | J- | |
|---|---|---|---|---|---|---|---|---|---|---|
| ID | V-J-REGION | L-FR1 | CDRL1 | L-FR2 | CDRL2 | L-FR3 | CDRL3 | TION | REGION | L-FR4 |
| AHH | DTTVTQSHKF | DTTV | QDVST | VAWY | SAS | YRYT | QQHYS | CQQHY | WTFGGG | FGGG |
| 03703 | MSTSVGDRVS | TQSH | A (SEQ | QQKP | (SEQ ID | GVPD | TPWT | STPWT | TKLEIK | TKLEI |
| | ITCKASQDVST | KFMS | ID NO: | GQSP | NO: 13) | RFTGS | (SEQ ID | F (SEQ | (SEQ ID | K |
| | AVAWYQQKP | TSVG | 1209) | KLLIY | | GSGT | NO: | ID NO: | NO: 1467) | (SEQ |
| | GQSPKLLIYSA | DRVSI | | (SEQ | | DFTFT | 1264) | 656) | | ID NO: |
| | SYRYTGVPDR | TCKA | | ID NO: | | ISSVQ | | | | 959) |
| | FTGSGSGTDFT | S (SEQ | | 1427) | | AEDL | | | | |
| | FTISSVQAEDL | ID NO: | | | | AVYY | | | | |
| | AVYYCQQHY | 840) | | | | C | | | | |
| | STPWTFGGGT | | | | | (SEQ | | | | |
| | KLEIK (SEQ ID | | | | | ID NO: | | | | |
| | NO: 841) | | | | | 1491) | | | | |
| AHH | DILMTQSPSSL | DILM | QSLLY | LAWY | WAS | TRES | QQYYS | CQQYY | WTFGGG | FGGG |
| 03704 | AVSVGEKVT | TQSPS | SSNQK | QQKP | (SEQ ID | GVPD | YPWT | SYPWT | TKLEMK | TKLE |
| | MSCKSSQSLL | SLAV | NY | GQSP | NO: | RFTGS | (SEQ ID | F (SEQ | (SEQ ID | MK |
| | YSSNQKNYLA | SVGE | (SEQ ID | KLLIY | 1450) | GSGT | NO: | ID NO: | NO: 1468) | (SEQ |
| | WYQQKPGQSP | KVTM | NO: | (SEQ | | DFTLT | 1294) | 676) | | ID NO: |
| | KLLIYWASTR | SCKSS | 1304) | ID NO: | | ISSVK | | | | 961) |
| | ESGVPDRFTG | (SEQ | | 1066) | | AEDL | | | | |
| | SGSGTDFTLTI | ID NO: | | | | AVYY | | | | |
| | SSVKAEDLAV | 708) | | | | C | | | | |
| | YYCQQYYSYP | | | | | (SEQ | | | | |
| | WTFGGGTKLE | | | | | ID NO: | | | | |
| | MK (SEQ ID | | | | | 1411) | | | | |
| | NO: 709) | | | | | | | | | |
| AHH | DIQMTQTTSSL | DIQM | QGISN | LNWY | YTS | SLHS | QQYSK | CQQYS | WTFGGG | FGGG |
| 03706 | SASLGDRVTIS | TQTTS | Y (SEQ | QQKP | (SEQ ID | GVPS | LPWT | KLPWT | TKLEMK | TKLE |
| | CSASQGISNYL | SLSAS | ID NO: | DGTV | NO: | RFSGS | (SEQ ID | F (SEQ | (SEQ ID | MK |
| | NWYQQKPDG | LGDR | 1211) | KLLIY | 1495) | GSGT | NO: | ID NO: | NO: 1468) | (SEQ |
| | TVKLLIYYTSS | VTISC | | (SEQ | | DYSL | 1289) | 672) | | ID NO: |
| | LHSGVPSRFSG | SAS | | ID NO: | | TISNL | | | | 961) |
| | SGSGTDYSLTI | (SEQ | | 1088) | | EPEDI | | | | |
| | SNLEPEDIATY | ID NO: | | | | ATYY | | | | |
| | YCQQYSKLPW | 761) | | | | C | | | | |
| | TFGGGTKLEM | | | | | (SEQ | | | | |
| | K (SEQ ID NO: | | | | | ID NO: | | | | |
| | 762) | | | | | 1368) | | | | |
| AHH | DIQMNQSPSY | DIQM | KSISK | LAWY | SGS | TLQS | QQHNE | CQQHN | TFGGGT | FGGG |
| 03707 | LAASPGETITI | NQSPS | Y (SEQ | QEKP | (SEQ ID | GIPSR | YPWT | EYPWT | KLEIK | TKLEI |
| | NCRASKSISKY | YLAA | ID NO: | GKTN | NO: | FSGSG | (SEQ ID | F (SEQ | (SEQ ID | K |
| | LAWYQEKPG | SPGET | 1059) | KLLIY | 1365) | SGTD | NO: | ID NO: | NO: 1393) | (SEQ |
| | KTNKLLIYSGS | ITINC | | (SEQ | | FTLTI | 1257) | 651) | | ID NO: |
| | TLQSGIPSRFS | RAS | | ID NO: | | SSLEP | | | | 959) |
| | GSGSGTDFTL | (SEQ | | 1064) | | EDFA | | | | |
| | TISSLEPEDFA | ID NO: | | | | MYYC | | | | |
| | MYYCQQHNE | 716) | | | | (SEQ | | | | |
| | YPWTFGGGTK | | | | | ID NO: | | | | |
| | LEIK (SEQ ID | | | | | 1405) | | | | |
| | NO: 717) | | | | | | | | | |
| AHH | DVVMTQTPLT | DVVM | QSLLD | LNWL | LVS | KLDS | WQGT | CWQG | TFGGGT | FGGG |
| 03708 | LSVTIGQPASI | TQTPL | SDGKT | LQRP | (SEQ ID | GVPD | HFPRT | THFPR | KLEIK | TKLEI |
| | SCKSSQSLLDS | TLSVT | Y (SEQ | GQSP | NO: | RFTGS | (SEQ ID | TF | (SEQ ID | K |
| | DGKTYLNWL | IGQPA | ID NO: | KRLIY | 1116) | GSGT | NO: | (SEQ ID | NO: 1393) | (SEQ |
| | LQRPGQSPKR | SISCK | 1301) | (SEQ | | DFTL | 1464) | NO: | | ID NO: |
| | LIYLVSKLDSG | SS | | ID NO: | | KINR | | 688) | | 959) |
| | VPDRFTGSGS | (SEQ | | 1083) | | VEAE | | | | |
| | GTDFTLKINR | ID NO: | | | | DLGV | | | | |
| | VEAEDLGVYY | 904) | | | | YYC | | | | |
| | CWQGTHFPRT | | | | | (SEQ | | | | |
| | FGGGTKLEIK | | | | | ID NO: | | | | |
| | (SEQ ID NO: | | | | | 1055) | | | | |
| | 905) | | | | | | | | | |
| AHH | DIVMTQSPKS | DIVM | ENVGT | VSWY | GAS | NRYT | GQSYS | CGQSY | YTFGGG | FGGG |
| 03709 | MSMSVGERVT | TQSP | Y (SEQ | QQKP | (SEQ ID | GVPD | YPYT | SYPYT | TKLEIK | TKLEI |
| | LSCKASENVG | KSMS | ID NO: | EQSP | NO: | RFTGS | (SEQ ID | F (SEQ | (SEQ ID | K |
| | TYVSWYQQK | MSVG | 933) | KLLIY | 975) | GSAT | NO: | ID NO: | NO: 1493) | (SEQ |
| | PEQSPKLLIYG | ERVT | | (SEQ | | DFTLT | 981) | 624) | | ID NO: |
| | ASNRYTGVPD | LSCK | | ID NO: | | ISSVQ | | | | 959) |
| | RFTGSGSATD | AS | | 1446) | | AEDL | | | | |
| | FTLTISSVQAE | (SEQ | | | | ADYH | | | | |
| | DLADYHCGQS | ID NO: | | | | C | | | | |
| | YSYPYTFGGG | 808) | | | | (SEQ | | | | |
| | TKLEIK (SEQ | | | | | ID NO: | | | | |
| | ID NO: 810) | | | | | 1162) | | | | |
| AHH | DIVMTQSQKF | DIVM | QNVRT | VAWY | MAS | NRHT | LQHW | CLQH | YTFGGG | FGGG |
| 03710 | MSTSVGDRVT | TQSQ | A (SEQ | HQKP | (SEQ ID | GVPD | NYPYT | WNYP | TKLEIK | TKLEI |
| | ITCKASQNVR | KFMS | ID NO: | GQSP | NO: | RFTGS | (SEQ ID | YTF | (SEQ ID | K |
| | TAVAWYHQK | TSVG | 1247) | KALIY | 1122) | GSGA | NO: | (SEQ ID | NO: 1493) | (SEQ |
| | PGQSPKALIY | DRVTI | | (SEQ | | DFTLT | 1093) | NO: | | ID NO: |
| | MASNRHTGVP | TCKA | | ID NO: | | ITNVQ | | 630) | | 959) |
| | DRFTGSGSGA | S (SEQ | | 1421) | | SEDL | | | | |
| | DFTLTITNVQS | ID NO: | | | | ADYF | | | | |
| | EDLADYFCLQ | 826) | | | | C | | | | |
| | HWNYPYTFG | | | | | (SEQ | | | | |
| | GGTKLEIK | | | | | ID NO: | | | | |
| | (SEQ ID NO: | | | | | 1161) | | | | |
| | 827) | | | | | | | | | |
| AHH | DIQMTQTPKF | DIQM | QSVSN | VAWY | YAS | NRYT | QQDYS | CQQDY | WTFGGG | FGGG |
| 03711 | LLVSAGDRVT | TQTP | D (SEQ | QQKP | (SEQ ID | GVPD | SPWT | SSPWT | TKLEIK | TKLEI |
| | ITCKASQSVSN | KFLL | ID NO: | GQSP | NO: | RFTGS | (SEQ ID | F (SEQ | (SEQ ID | K |
| | DVAWYQQKP | VSAG | 1310) | KLLIY | 1476) | GYGT | NO: | ID NO: | NO: 1467) | (SEQ |
| | GQSPKLLIYY | DRVTI | | (SEQ | | DFTFT | 1251) | 647) | | ID NO: |
| | ASNRYTGVPD | TCKA | | ID NO: | | ISTVQ | | | | 959) |
| | RFTGSGYGTD | S (SEQ | | 1427) | | AEDL | | | | |
| | FTFISTVQAE | ID NO: | | | | AVYF | | | | |
| | DLAVYFCQQD | 746) | | | | C | | | | |
| | YSSPWTFGGG | | | | | (SEQ | | | | |
| | TKLEIK (SEQ | | | | | ID NO: | | | | |
| | ID NO: 747) | | | | | 1164) | | | | |
| AHH | DIQMTQTPSSL | DIQM | QEISG | LSWL | AAS | TLDS | LQYIS | CLQYI | TFGGGT | FGGG |
| 03713 | SASLGERVSLT | TQTPS | Y (SEQ | QQKP | (SEQ ID | GVPK | YPRT | SYPRT | KLEIK | TKLEI |
| | CRASQEISGYL | SLSAS | ID NO: | DGTIK | NO: | RFSGS | (SEQ ID | F (SEQ | (SEQ ID | K |
| | SWLQQKPDGT | LGER | 1210) | RLIY | 599) | RSGS | NO: | ID NO: | NO: 1393) | (SEQ |
| | IKRLIYAASTL | VSLT | | (SEQ | | DYSL | 1107) | 640) | | ID NO: |
| | DSGVPKRFSG | CRAS | | ID NO: | | TISSL | | | | 959) |
| | SRSGSDYSLTI | (SEQ | | 1111) | | ESEDF | | | | |
| | SSLESEDFADY | ID NO: | | | | ADYY | | | | |
| | YCLQYISYPRT | 753) | | | | C | | | | |
| | FGGGTKLEIK | | | | | (SEQ | | | | |
| | (SEQ ID NO: | | | | | ID NO: | | | | |
| | 756) | | | | | 1402) | | | | |
| AHH | DIVMTQSQKF | DIVM | QNVGT | VAWY | SAS | YRYS | QQYNS | CQQYN | TFGGGT | FGGG |
| 03714 | MSTSVGDRVS | TQSQ | N (SEQ | QQKP | (SEQ ID | GVPD | YPRT | SYPRT | KLEIK | TKLEI |
| | VTCKASQNVG | KFMS | ID NO: | GQSP | NO: 13) | RFTGS | (SEQ ID | F (SEQ | (SEQ ID | K |
| | TNVAWYQQK | TSVG | 12) | KALIY | | GSGT | NO: | ID NO: | NO: 1393) | (SEQ |
| | PGQSPKALIYS | DRVS | | (SEQ | | DFTLT | 1286) | 670) | | ID NO: |
| | ASYRYSGVPD | VTCK | | ID NO: | | ISNVQ | | | | 959) |
| | RFTGSGSGTD | AS | | 1425) | | SEDL | | | | |
| | FTLTISNVQSE | (SEQ | | | | AEYL | | | | |
| | DLAEYLCQQY | ID NO: | | | | C | | | | |
| | NSYPRTFGGG | 821) | | | | (SEQ | | | | |
| | TKLEIK (SEQ | | | | | ID NO: | | | | |
| | ID NO: 824) | | | | | 1485) | | | | |
| AHH | DIQMTQSPAS | DIQM | ENIYS | LAWY | AAT | NLAD | QHFW | CQHF | TFGGGT | FGGG |
| 03715 | LSVSVGETVTI | TQSP | N (SEQ | QQKQ | (SEQ ID | GVPS | GTPPT | WGTPP | KLEIK | TKLEI |
| | TCRASENIYSN | ASLS | ID NO: | GKSP | NO: | RFSGS | (SEQ ID | TF | (SEQ ID | K |
| | LAWYQQKQG | VSVG | 929) | QLLV | 600) | GSGT | NO: | (SEQ ID | NO: 1393) | (SEQ |
| | KSPQLLVYAA | ETVTI | | (SEQ | | QYSL | 1212) | NO: | | ID NO: |
| | TNLADGVPSR | TCRA | | ID NO: | | KINSL | | 642) | | 959) |
| | FSGSGSGTQY | S (SEQ | | 1069) | | QSED | | | | |
| | SLKINSLQSED | ID NO: | | | | FGSY | | | | |
| | FGSYYCQHFW | 723) | | | | YC | | | | |
| | GTPPTFGGGT | | | | | (SEQ | | | | |
| | KLEIK (SEQ ID | | | | | ID NO: | | | | |
| | NO: 724) | | | | | 1143) | | | | |
| AHH | DVVMTQTPLT | DVVM | QSLLD | LNWL | LVS | KLDS | WQGT | CWQG | TFGGGT | FGGG |
| 03716 | LSVTIGQPASI | TQTPL | SDGKT | LQRP | (SEQ ID | GVPD | HFRT | THFRT | KLEMK | TKLE |
| | SCKSSQSLLDS | TLSVT | Y (SEQ | GQSP | NO: | RFTGS | (SEQ ID | F (SEQ | (SEQ ID | MK |
| | DGKTYLNWL | IGQPA | ID NO: | KRLIY | 1116) | GSGT | NO: | ID NO: | NO: 1394) | (SEQ |
| | LQRPGQSPKR | SISCK | 1301) | (SEQ | | DFTL | 1466) | 690) | | ID NO: |
| | LIYLVSKLDSG | SS | | ID NO: | | KISRV | | | | 961) |
| | VPDRFTGSGS | (SEQ | | 1083) | | EAED | | | | |
| | GTDFTLKISRV | ID NO: | | | | LGVY | | | | |
| | EAEDLGVYYC | 904) | | | | YC | | | | |
| | WQGTHFRTFG | | | | | (SEQ | | | | |
| | GGTKLEMK | | | | | ID NO: | | | | |
| | (SEQ ID NO: | | | | | 1056) | | | | |
| | 906) | | | | | | | | | |
| AHH | DVVLTQTPLS | DVVL | QSLVH | LHWY | KVS | NRFS | SQSTH | CSQST | LTFGAG | FGAG |
| 03717 | LPVSLGDQVSI | TQTPL | NNGNT | LQKP | (SEQ ID | GVPD | VPLT | HVPLT | TKLEIK | TKLEI |
| | SCRSGQSLVH | SLPVS | Y (SEQ | GQSP | NO: | RFSGS | (SEQ ID | F (SEQ | (SEQ ID | K |
| | NNGNTYLHW | LGDQ | ID NO: | KLLIY | 1062) | GSGT | NO: | ID NO: | NO: 1114) | (SEQ |
| | YLQKPGQSPK | VSISC | 1305) | (SEQ | | DFTL | 1370) | 679) | | ID NO: |
| | LLIYKVSNRFS | RSG | | ID NO: | | KISRV | | | | 958) |
| | GVPDRFSGSG | (SEQ | | 1076) | | EAED | | | | |
| | SGTDFTLKISR | ID NO: | | | | LGVY | | | | |
| | VEAEDLGVYF | 881) | | | | FC | | | | |
| | CSQSTHVPLTF | | | | | (SEQ | | | | |
| | GAGTKLEIK | | | | | ID NO: | | | | |
| | (SEQ ID NO: | | | | | 1157) | | | | |
| | 882) | | | | | | | | | |
| AHH | DIQMTQSTSSL | DIQM | QDISN | LNWY | FTS | RLYS | QQGNT | CQQGN | TFGGGT | FGGG |
| 03718 | SASLGDRVTIS | TQSTS | Y (SEQ | QQKP | (SEQ ID | GVPS | LPRT | TLPRT | KLEIK | TKLEI |
| | CRASQDISNY | SLSAS | ID NO: | DGTV | NO: | RFSGS | (SEQ ID | F (SEQ | (SEQ ID | K |
| | LNWYQQKPD | LGDR | 27) | KLLIY | 972) | GSGA | NO: | ID NO: | NO: 1393) | (SEQ |
| | GTVKLLIYFTS | VTISC | | (SEQ | | DYSL | 1254) | 648) | | ID NO: |
| | RLYSGVPSRFS | RAS | | ID NO: | | TISNL | | | | 959) |
| | GSGSGADYSL | (SEQ | | 1088) | | EQEDI | | | | |
| | TISNLEQEDIA | ID NO: | | | | ATYF | | | | |
| | TYFCQQGNTL | 738) | | | | C | | | | |
| | PRTFGGGTKL | | | | | (SEQ | | | | |
| | EIK (SEQ ID | | | | | ID NO: | | | | |
| | NO: 739) | | | | | 1358) | | | | |
| AHH | DIQMNQSPSSL | DIQM | QDIGS | LNWL | ATS | SLDS | LQYAS | CLQYA | YTFGGG | FGGG |
| 03719 | SASLGERVSLT | NQSPS | S (SEQ | QQEP | (SEQ ID | GVPK | SPYT | SSPYTF | TKLEIK | TKLEI |
| | CRASQDIGSSL | SLSAS | ID NO: | DGTIK | NO: | RFSGS | (SEQ ID | (SEQ ID | (SEQ ID | K |
| | NWLQQEPDGT | LGER | 1199) | RLIY | 618) | RSGS | NO: | NO: | NO: 1493) | (SEQ |
| | IKRLIYATSSL | VSLT | | (SEQ | | DYSL | 1101) | 637) | | ID NO: |
| | DSGVPKRFSG | CRAS | | ID NO: | | TISSL | | | | 959) |
| | SRSGSDYSLTI | (SEQ | | 1085) | | ESEDF | | | | |
| | SSLESEDFVDY | ID NO: | | | | VDYY | | | | |
| | YCLQYASSPY | 714) | | | | C | | | | |
| | TFGGGTKLEIK | | | | | (SEQ | | | | |
| | (SEQ ID NO: | | | | | ID NO: | | | | |
| | 715) | | | | | 1366) | | | | |
| AHH | DVVMTQTTSS | DVVM | QDISN | LNWY | YTS | RLHS | QQVYT | CQQVY | WTFGGG | FGGG |
| 03720 | LSASLGDRVTI | TQTTS | Y (SEQ | QQRP | (SEQ ID | GVPS | LPWT | TLPWT | TKLEIK | TKLEI |
| | SCRASQDISNY | SLSAS | ID NO: | DGTV | NO: | RFSGS | (SEQ ID | F (SEQ | (SEQ ID | K |
| | LNWYQQRPD | LGDR | 27) | KLLIY | 1495) | GSGT | NO: | ID NO: | NO: 1467) | (SEQ |
| | GTVKLLIYYTS | VTISC | | (SEQ | | DFSLT | 1273) | 663) | | ID NO: |
| | RLHSGVPSRFS | RAS | | ID NO: | | ISNLE | | | | 959) |
| | GSGSGTDFSLT | (SEQ | | 1090) | | QEDIA | | | | |
| | ISNLEQEDIAT | ID NO: | | | | TYFC | | | | |
| | YFCQQVYTLP | 913) | | | | (SEQ | | | | |
| | WTFGGGTKLE | | | | | ID NO: | | | | |
| | IK (SEQ ID NO: | | | | | 1351) | | | | |
| | 914) | | | | | | | | | |
| AHH | DIQMTQSPSSL | DIQM | QEISG | LSWL | AAS | TLDS | LQYAS | CLQYA | TFGGGT | FGGG |
| 03721 | SASLGERVSLT | TQSPS | Y (SEQ | QQKP | (SEQ ID | GVPK | YPRT | SYPRT | KLEIK | TKLEI |
| | CRASQEISGYL | SLSAS | ID NO: | DGTIK | NO: | RFSGS | (SEQ ID | F (SEQ | (SEQ ID | K |
| | SWLQQKPDGT | LGER | 1210) | RLIY | 599) | RSGS | NO: | ID NO: | NO: 1393) | (SEQ |
| | IKRLIYAASTL | VSLT | | (SEQ | | DYSL | 1102) | 638) | | ID NO: |
| | DSGVPKRFSG | CRAS | | ID NO: | | TISSL | | | | 959) |
| | SRSGSDYSLTI | (SEQ | | 1111) | | ESEDF | | | | |
| | SSLESEDFADY | ID NO: | | | | ADYY | | | | |
| | YCLQYASYPR | 726) | | | | C | | | | |
| | TFGGGTKLEIK | | | | | (SEQ | | | | |
| | (SEQ ID NO: | | | | | ID NO: | | | | |
| | 728) | | | | | 1402) | | | | |
| AHH | DTTVTQSPAS | DTTV | QSVDY | MNW | AAS | NLES | QQSNE | CQQSN | YTFGGG | FGGG |
| 03724 | LAVSLGQRAT | TQSP | DGDSY | YQQK | (SEQ ID | GIPAR | DPYT | EDPYT | TKLEMK | TKLE |
| | ISCKASQSVD | ASLA | (SEQ ID | PGQPP | NO: | FSGSG | (SEQ ID | F (SEQ | (SEQ ID | MK |
| | YDGDSYMNW | VSLG | NO: | KLLIY | 599) | SGTD | NO: | ID NO: | NO: 1494) | (SEQ |
| | YQQKPGQPPK | QRATI | 1308) | (SEQ | | FTLNI | 1270) | 662) | | ID NO: |
| | LLIYAASNLES | SCKA | | ID NO: | | HPVE | | | | 961) |
| | GIPARFSGSGS | S (SEQ | | 1135) | | EEDA | | | | |
| | GTDFTLNIHPV | ID NO: | | | | ATYY | | | | |
| | EEEDAATYYC | 847) | | | | C | | | | |
| | QQSNEDPYTF | | | | | (SEQ | | | | |
| | GGGTKLEMK | | | | | ID NO: | | | | |
| | (SEQ ID NO: | | | | | 1148) | | | | |
| | 848) | | | | | | | | | |
| AHH | DVVMTQTPLT | DVVM | QSLLD | LNWL | LVS | KLDS | WQGT | CWQG | WTFGGG | FGGG |
| 03725 | LSVTIGQPASI | TQTPL | SDGKT | LQRP | (SEQ ID | GVPD | HFPWT | THFPW | TKLEIK | TKLEI |
| | SCKSSQSLLDS | TLSVT | Y (SEQ | GQSP | NO: | RFTGS | (SEQ ID | TF | (SEQ ID | K |
| | DGKTYLNWL | IGQPA | ID NO: | KRLIY | 1116) | GSGT | NO: | (SEQ ID | NO: 1467) | (SEQ |
| | LQRPGQSPKR | SISCK | 1301) | (SEQ | | DFTL | 1465) | NO: | | ID NO: |
| | LIYLVSKLDSG | SS | | ID NO: | | KISRV | | 689) | | 959) |
| | VPDRFTGSGS | (SEQ | | 1083) | | EAED | | | | |
| | GTDFTLKISRV | ID NO: | | | | LGVY | | | | |
| | EAEDLGVYYC | 904) | | | | YC | | | | |
| | WQGTHFPWTF | | | | | (SEQ | | | | |
| | GGGTKLEIK | | | | | ID NO: | | | | |
| | (SEQ ID NO: | | | | | 1056) | | | | |
| | 907) | | | | | | | | | |
| AHH | DVVLTQTPLS | DVVL | QSLVH | LHWY | KVS | NRFS | SQSTH | CSQST | WTFGGG | FGGG |
| 03727 | LPVSLGDQASI | TQTPL | SNGNT | LQKP | (SEQ ID | GVPD | VPWT | HVPWT | TKLEIK | TKLEI |
| | SCRSSQSLVHS | SLPVS | Y (SEQ | GQSP | NO: | RFSGS | (SEQ ID | F (SEQ | (SEQ ID | K |
| | NGNTYLHWY | LGDQ | ID NO: | KLLIY | 1062) | GSGT | NO: | ID NO: | NO: 1467) | (SEQ |
| | LQKPGQSPKL | ASISC | 1306) | (SEQ | | DFTL | 1374) | 682) | | ID NO: |
| | LIYKVSNRFSG | RSS | | ID NO: | | KISRV | | | | 959) |
| | VPDRFSGSGS | (SEQ | | 1076) | | EAED | | | | |
| | GTDFTLKISRV | ID NO: | | | | LGVY | | | | |
| | EAEDLGVYFC | 874) | | | | FC | | | | |
| | SQSTHVPWTF | | | | | (SEQ | | | | |
| | GGGTKLEIK | | | | | ID NO: | | | | |
| | (SEQ ID NO: | | | | | 1157) | | | | |
| | 876) | | | | | | | | | |
| AHH | DTTVTQSHRF | DTTV | QDVNT | VAWY | SAS | YRYT | QQHFN | CQQHF | YTFGGG | FGGG |
| 03728 | MSTSVEDRVT | TQSH | A (SEQ | QHKP | (SEQ ID | GVPD | SPYT | NSPYT | TKLEIK | TKLEI |
| | IACKASQDVN | RFMS | ID NO: | GQSP | NO: 13) | RFTGS | (SEQ ID | F (SEQ | (SEQ ID | K |
| | TAVAWYQHK | TSVE | 1206) | KLLIY | | GSRT | NO: | ID NO: | NO: 1493) | (SEQ |
| | PGQSPKLLIYS | DRVTI | | (SEQ | | DFTFT | 1256) | 650) | | ID NO: |
| | ASYRYTGVPD | ACKA | | ID NO: | | ISSVQ | | | | 959) |
| | RFTGSGSRTDF | S (SEQ | | 1422) | | AEDL | | | | |
| | TFTISSVQAED | ID NO: | | | | AVYY | | | | |
| | LAVYYCQQHF | 843) | | | | C | | | | |
| | NSPYTFGGGT | | | | | (SEQ | | | | |
| | KLEIK (SEQ ID | | | | | ID NO: | | | | |
| | NO: 844) | | | | | 1492) | | | | |
| AHH | DIVMSQSPASL | DIVM | QSVDY | MNW | ATS | NLES | QQSNE | CQQSN | LTFGAG | FGAG |
| 03729 | AVSLGQRATIS | SQSPA | DGDSY | YQQK | (SEQ ID | GIPAR | DPLT | EDPLT | TKLEIK | TKLEI |
| | CKASQSVDYD | SLAV | (SEQ ID | PGQPP | NO: | FSGSG | (SEQ ID | F (SEQ | (SEQ ID | K |
| | GDSYMNWYQ | SLGQ | NO: | KLLIY | 618) | SGTD | NO: | ID NO: | NO: 1114) | (SEQ |
| | QKPGQPPKLLI | RATIS | 1308) | (SEQ | | FTLNI | 1268) | 660) | | ID NO: |
| | YATSNLESGIP | CKAS | | ID NO: | | HPVE | | | | 958) |
| | ARFSGSGSGT | (SEQ | | 1135) | | EEDA | | | | |
| | DFTLNIHPVEE | ID NO: | | | | ATYY | | | | |
| | EDAATYYCQQ | 786) | | | | C | | | | |
| | SNEDPLTFGA | | | | | (SEQ | | | | |
| | GTKLEIK (SEQ | | | | | ID NO: | | | | |
| | ID NO: 787) | | | | | 1148) | | | | |
| AHH | DIQMTRSPSSL | DIQM | QDIGS | LNWL | ATS | SLDS | LQYAS | CLQYA | WTFGGG | FGGG |
| 03730 | SASLGERVSLT | TRSPS | S (SEQ | QQEP | (SEQ ID | GVPK | SPWT | SSPWT | TKLEIK | TKLEI |
| | CRASQDIGSSL | SLSAS | ID NO: | DGTIK | NO: | RFSGS | (SEQ ID | F (SEQ | (SEQ ID | K |
| | NWLQQEPDGT | LGER | 1199) | RLIY | 618) | RSGS | NO: | ID NO: | NO: 1467) | (SEQ |
| | IKRLIYATSSL | VSLT | | (SEQ | | DYSL | 1100) | 636) | | ID NO: |
| | DSGVPKRFSG | CRAS | | ID NO: | | TISSL | | | | 959) |
| | SRSGSDYSLTI | (SEQ | | 1085) | | ESEDF | | | | |
| | SSLESEDFVDY | ID NO: | | | | VDYY | | | | |
| | YCLQYASSPW | 764) | | | | C | | | | |
| | TFGGGTKLEIK | | | | | (SEQ | | | | |
| | (SEQ ID NO: | | | | | ID NO: | | | | |
| | 765) | | | | | 1366) | | | | |
| AHH | DIVMTQSPAI | DIVM | SSVSY | MHW | DTS | KLAS | QQWSS | CQQW | FGGGTK | FGGG |
| 03731 | MSASPGEKVT | TQSP | (SEQ ID | YQQK | (SEQ ID | GVPA | NPPT | SSNPPT | LEIK | TKLEI |
| | MTCSASSSVS | AIMS | NO: | SGTSP | NO: | RFSGS | (SEQ ID | F (SEQ | (SEQ ID | K |
| | YMHWYQQKS | ASPG | 1385) | KRWI | 839) | GSGT | NO: | ID NO: | NO: 959) | (SEQ |
| | GTSPKRWIYD | EKVT | | (SEQ | | SYSLT | 1274) | 664) | | ID NO: |
| | TSKLASGVPA | MTCS | | ID NO: | | ISSME | | | | 959) |
| | RFSGSGSGTSY | AS | | 1131) | | AEDA | | | | |
| | SLTISSMEAED | (SEQ | | | | ATYY | | | | |
| | AATYYCQQW | ID NO: | | | | C | | | | |
| | SSNPPTFGGGT | 802) | | | | (SEQ | | | | |
| | KLEIK (SEQ ID | | | | | ID NO: | | | | |
| | NO: 803) | | | | | 1052) | | | | |
| AHH | DTTVTQSPAI | DTTV | SSIGY | MHW | DTS | KLAS | HQRGS | CHQRG | WTFGGG | FGGG |
| 03732 | MSASPGEKVT | TQSP | (SEQ ID | YQQK | (SEQ ID | GVPA | YPWT | SYPWT | TKLEIK | TKLEI |
| | MTCSASSSIGY | AIMS | NO: | PGTSP | NO: | RFSGS | (SEQ ID | F (SEQ | (SEQ ID | K |
| | MHWYQQKPG | ASPG | 1381) | KRWI | 839) | GSGT | NO: | ID NO: | NO: 1467) | (SEQ |
| | TSPKRWIYDT | EKVT | | (SEQ | | SYSLT | 999) | 626) | | ID NO: |
| | SKLASGVPAR | MTCS | | ID NO: | | ISSME | | | | 959) |
| | FSGSGSGTSYS | AS | | 1130) | | AEDA | | | | |
| | LTISSMEAEDA | (SEQ | | | | ATYY | | | | |
| | ATYYCHQRGS | ID NO: | | | | C | | | | |
| | YPWTFGGGTK | 846) | | | | (SEQ | | | | |
| | LEIK (SEQ ID | | | | | ID NO: | | | | |
| | NO: 846) | | | | | 1052) | | | | |
| AHH | DVVLTQTPLS | DVVL | QSIVH | LEWY | KVS | NRFS | FQGSH | CFQGS | YTFGGG | FGGG |
| 03733 | LPVSLGDQASI | TQTPL | SNGNT | LQKP | (SEQ ID | GVPD | VPYT | HVPYT | TKLEIK | TKLEI |
| | SCRSSQSIVHS | SLPVS | Y (SEQ | GQSP | NO: | RFSGS | (SEQ ID | F (SEQ | (SEQ ID | K |
| | NGNTYLEWY | LGDQ | ID NO: | KLLIY | 1062) | GSGT | NO: | ID NO: | NO: 1493) | (SEQ |
| | LQKPGQSPKL | ASISC | 1299) | (SEQ | | DFTL | 970) | 621) | | ID NO: |
| | LIYKVSNRFSG | RSS | | ID NO: | | KISRV | | | | 959) |
| | VPDRFSGSGS | (SEQ | | 1071) | | EAED | | | | |
| | GTDFTLKISRV | ID NO: | | | | LGVY | | | | |
| | EAEDLGVYYC | 874) | | | | YC | | | | |
| | FQGSHVPYTF | | | | | (SEQ | | | | |
| | GGGTKLEIK | | | | | ID NO: | | | | |
| | (SEQ ID NO: | | | | | 1158) | | | | |
| | 875) | | | | | | | | | |
| AHH | DTTVTQSHKF | DTTV | QDVST | VAWY | SAS | YRYT | QQHYS | CQQHY | WTFGGG | FGGG |
| 03734 | MSTSVGDRVS | TQSH | A (SEQ | QQKP | (SEQ ID | GVPD | TPWT | STPWT | TKLEIK | TKLEI |
| | ITCKASQDVST | KFMS | ID NO: | GQSP | NO: 13) | RFTGS | (SEQ ID | F (SEQ | (SEQ ID | K |
| | AVAWYQQKP | TSVG | 1209) | KLLIY | | GSGT | NO: | ID NO: | NO: 1467) | (SEQ |
| | GQSPKLLIYSA | DRVSI | | (SEQ | | DFTFT | 1264) | 656) | | ID NO: |
| | SYRYTGVPDR | TCKA | | ID NO: | | ISSVQ | | | | 959) |
| | FTGSGSGTDFT | S (SEQ | | 1427) | | AEDL | | | | |
| | FTISSVQAEDL | ID NO: | | | | AVYY | | | | |
| | AVYYCQQHY | 840) | | | | C | | | | |
| | STPWTFGGGT | | | | | (SEQ | | | | |
| | KLEIK (SEQ ID | | | | | ID NO: | | | | |
| | NO: 841) | | | | | 1491) | | | | |
| AHH | DIVLTQSPASL | DIVLT | ESVDN | MNWF | AAS | NQGS | QQSKE | CQQSK | YTFGGG | FGGG |
| 03735 | AVSLGQRATIS | QSPAS | YGISF | QKKP | (SEQ ID | GVPA | VPYT | EVPYT | TKLEIK | TKLEI |
| | CRASESVDNY | LAVS | (SEQ ID | GQPP | NO: | RFSGS | (SEQ ID | F (SEQ | (SEQ ID | K |
| | GISFMNWFQK | LGQR | NO: | KLLIY | 599) | GSGT | NO: | ID NO: | NO: 1493) | (SEQ |
| | KPGQPPKLLIY | ATISC | 934) | (SEQ | | DFSL | 1267) | 659) | | ID NO: |
| | AASNQGSGVP | RAS | | ID NO: | | NIHP | | | | 959) |
| | ARFSGSGSGT | (SEQ | | 1133) | | MEED | | | | |
| | DFSLNIHPMEE | ID NO: | | | | DTAM | | | | |
| | DDTAMYFCQ | 768) | | | | YFC | | | | |
| | QSKEVPYTFG | | | | | (SEQ | | | | |
| | GGTKLEIK | | | | | ID NO: | | | | |
| | (SEQ ID NO: | | | | | 1152) | | | | |
| | 769) | | | | | | | | | |
| AHH | DNVLTQSPSSL | DNVL | QSLLY | LAWY | WAS | TRES | QQYYS | CQQYY | TFGGGT | FGGG |
| 03736 | AVSVGEKVT | TQSPS | SSNQK | QQKP | (SEQ ID | GVPD | YRT | SYRTF | KLEIK | TKLEI |
| | MSCKSSQSLL | SLAV | NY | GQSP | NO: | RFTGS | (SEQ ID | (SEQ ID | (SEQ ID | K |
| | YSSNQKNYLA | SVGE | (SEQ ID | KLLIY | 1450) | GSGT | NO: | NO: | NO: 1393) | (SEQ |
| | WYQQKPGQSP | KVTM | NO: | (SEQ | | DFTLT | 1295) | 677) | | ID NO: |
| | KLLIYWASTR | SCKSS | 1304) | ID NO: | | ISSVK | | | | 959) |
| | ESGVPDRFTG | (SEQ | | 1066) | | AEDL | | | | |
| | SGSGTDFTLTI | ID NO: | | | | AVYY | | | | |
| | SSVKAEDLAV | 834) | | | | C | | | | |
| | YYCQQYYSYR | | | | | (SEQ | | | | |
| | TFGGGTKLEIK | | | | | ID NO: | | | | |
| | (SEQ ID NO: | | | | | 1411) | | | | |
| | 835) | | | | | | | | | |
| AHH | DIVMTQSPKS | DIVM | ENVAT | VSWY | GAS | NRYT | GQSYR | CGQSY | YTFGGG | FGGG |
| 03737 | MSMSVGERVT | TQSP | Y (SEQ | QQKP | (SEQ ID | GVPD | YPYT | RYPYT | TKLEMK | TKLE |
| | LSCKASENVA | KSMS | ID NO: | EQSP | NO: | RFTGS | (SEQ ID | F (SEQ | (SEQ ID | MK |
| | TYVSWYQQK | MSVG | 932) | KLLIY | 975) | GSAT | NO: | ID NO: | NO: 1494) | (SEQ |
| | PEQSPKLLIYG | ERVT | | (SEQ | | DFTLT | 979) | 622) | | ID NO: |
| | ASNRYTGVPD | LSCK | | ID NO: | | ISSVQ | | | | 961) |
| | RFTGSGSATD | AS | | 1446) | | AEDL | | | | |
| | FTLTISSVQAE | (SEQ | | | | ADYR | | | | |
| | DLADYRCGQS | ID NO: | | | | C | | | | |
| | YRYPYTFGGG | 808) | | | | (SEQ | | | | |
| | TKLEMK (SEQ | | | | | ID NO: | | | | |
| | ID NO: 809) | | | | | 1163) | | | | |
| AHH | DVVVTQTPLS | DVVV | QSLVH | LHWY | KVS | NRFS | SQSTH | CSQST | TFGGGT | FGGG |
| 03738 | LPVSLGDQASI | TQTPL | SNGNT | LQKP | (SEQ ID | GVPD | VPT | HVPTF | KLEIK | TKLEI |
| | SCRSSQSLVHS | SLPVS | Y (SEQ | GQSP | NO: | RFSGS | (SEQ ID | (SEQ ID | (SEQ ID | K |
| | NGNTYLHWY | LGDQ | ID NO: | KLLIY | 1062) | GSGT | NO: | NO: | NO: 1393) | (SEQ |
| | LQKPGQSPKL | ASISC | 1306) | (SEQ | | DFTL | 1373) | 681) | | ID NO: |
| | LIYKVSNRFSG | RSS | | ID NO: | | KISRV | | | | 959) |
| | VPDRFSGSGS | (SEQ | | 1076) | | EAED | | | | |
| | GTDFTLKISRV | ID NO: | | | | LGVY | | | | |
| | EAEDLGVYFC | 918) | | | | FC | | | | |
| | SQSTHVPTFG | | | | | (SEQ | | | | |
| | GGTKLEIK | | | | | ID NO: | | | | |
| | (SEQ ID NO: | | | | | 1157) | | | | |
| | 920) | | | | | | | | | |
| AHH | DVVVTQSPAS | DVVV | ESVDS | MHW | LAS | NLES | QQNNE | CQQNN | YTFGGG | FGGG |
| 03739 | LAVSLGQRAT | TQSP | YGNSF | YQQK | (SEQ ID | GVPA | DPYT | EDPYT | TKLEIK | TKLEI |
| | ISCRASESVDS | ASLA | (SEQ ID | PGQPP | NO: | RFSGS | (SEQ ID | F (SEQ | (SEQ ID | K |
| | YGNSFMHWY | VSLG | NO: | KLLIY | 1063) | GSRT | NO: | ID NO: | NO: 1493) | (SEQ |
| | QQKPGQPPKL | QRATI | 935) | (SEQ | | DFTLT | 1266) | 658) | | ID NO: |
| | LIYLASNLESG | SCRA | | ID NO: | | IDPVE | | | | 959) |
| | VPARFSGSGS | S (SEQ | | 1129) | | ADDA | | | | |
| | RTDFTLTIDPV | ID NO: | | | | ATYY | | | | |
| | EADDAATYYC | 916) | | | | C | | | | |
| | QQNNEDPYTF | | | | | (SEQ | | | | |
| | GGGTKLEIK | | | | | ID NO: | | | | |
| | (SEQ ID NO: | | | | | 1150) | | | | |
| | 917) | | | | | | | | | |
| AHH | DVVMTQSQKF | DVVM | QNVGT | VAWY | SAS | YRYS | QQYNS | CQQYN | YTFGGG | FGGG |
| 03740 | MSTSVGDRVN | TQSQ | N (SEQ | QQKP | (SEQ ID | GVPD | YPYT | SYPYT | TKLEIK | TKLEI |
| | VTCKASQNVG | KFMS | ID NO: | GQSP | NO: 13) | RFTGS | (SEQ ID | F (SEQ | (SEQ ID | K |
| | TNVAWYQQK | TSVG | 12) | KALIY | | GSGT | NO: 15) | ID NO: | NO: 1493) | (SEQ |
| | PGQSPKALIYS | DRVN | | (SEQ | | DFTLT | | 671) | | ID NO: |
| | ASYRYSGVPD | VTCK | | ID NO: | | ISNVQ | | | | 959) |
| | RFTGSGSGTD | AS | | 1425) | | SEDL | | | | |
| | FTLTISNVQSE | (SEQ | | | | AEYF | | | | |
| | DLAEYFCQQY | ID NO: | | | | C | | | | |
| | NSYPYTFGGG | 891) | | | | (SEQ | | | | |
| | TKLEIK (SEQ | | | | | ID NO: | | | | |
| | ID NO: 892) | | | | | 1483) | | | | |
| AHH | DVVMTQTPLS | DVVM | QSLVH | LHWY | KVS | NRFS | SQSTH | CSQST | WTFGGG | FGGG |
| 03741 | LPVSLGDQASI | TQTPL | SNGNT | LQKP | (SEQ ID | GVPD | VPWT | HVPWT | TKLEIK | TKLEI |
| | SCRSSQSLVHS | SLPVS | Y (SEQ | GQSP | NO: | RFSGS | (SEQ ID | F (SEQ | (SEQ ID | K |
| | NGNTYLHWY | LGDQ | ID NO: | KLLIY | 1062) | GSGT | NO: | ID NO: | NO: 1467) | (SEQ |
| | LQKPGQSPKL | ASISC | 1306) | (SEQ | | DFTL | 1374) | 682) | | ID NO: |
| | LIYKVSNRFSG | RSS | | ID NO: | | KISRV | | | | 959) |
| | VPDRFSGSGS | (SEQ | | 1076) | | EAED | | | | |
| | GTDFTLKISRV | ID NO: | | | | LGVY | | | | |
| | EAEDLGVYFC | 900) | | | | FC | | | | |
| | SQSTHVPWTF | | | | | (SEQ | | | | |
| | GGGTKLEIK | | | | | ID NO: | | | | |
| | (SEQ ID NO: | | | | | 1157) | | | | |
| | 901) | | | | | | | | | |
| AHH | DIVMTQSPKS | DIVM | ENVGT | VSWY | GAS | NRYT | GQSYS | CGQSY | YTFGGG | FGGG |
| 03742 | MSMSVGERVT | TQSP | Y (SEQ | QQKP | (SEQ ID | GVPD | YPYT | SYPYT | TKLEIK | TKLEI |
| | LSCKASENVG | KSMS | ID NO: | EQSP | NO: | RFTGS | (SEQ ID | F (SEQ | (SEQ ID | K |
| | TYVSWYQQK | MSVG | 933) | KLLIY | 975) | GSAT | NO: | ID NO: | NO: 1493) | (SEQ |
| | PEQSPKLLIYG | ERVT | | (SEQ | | DFTLT | 981) | 624) | | ID NO: |
| | ASNRYTGVPD | LSCK | | ID NO: | | ISSVQ | | | | 959) |
| | RFTGSGSATD | AS | | 1446) | | AEDL | | | | |
| | FTLTISSVQAE | (SEQ | | | | ADYH | | | | |
| | DLADYHCGQS | ID NO: | | | | C | | | | |
| | YSYPYTFGGG | 808) | | | | (SEQ | | | | |
| | TKLEIK (SEQ | | | | | ID NO: | | | | |
| | ID NO: 810) | | | | | 1162) | | | | |
| AHH | DVVMTQSHKF | DVVM | QDVST | VAWY | SAS | YRYT | QQHYS | CQQHY | WTFGRG | FGRG |
| 03743 | MSTSVGDRVS | TQSH | A (SEQ | QQKP | (SEQ ID | GVPD | TPPWT | STPPW | TKLEIK | TKLEI |
| | ITCKASQDVST | KFMS | ID NO: | GQSP | NO: 13) | RFTGS | (SEQ ID | TF | (SEQ ID | K |
| | AVAWYQQKP | TSVG | 1209) | KLLIY | | GSGT | NO: | (SEQ ID | NO: 1469) | (SEQ |
| | GQSPKLLIYSA | DRVSI | | (SEQ | | DFTFT | 1262) | NO: | | ID NO: |
| | SYRYTGVPDR | TCKA | | ID NO: | | ISSVQ | | 654) | | 964) |
| | FTGSGSGTDFT | S (SEQ | | 1427) | | AEDL | | | | |
| | FTISSVQAEDL | ID NO: | | | | AVYY | | | | |
| | AVYYCQQHY | 885) | | | | C | | | | |
| | STPPWTFGRG | | | | | (SEQ | | | | |
| | TKLEIK (SEQ | | | | | ID NO: | | | | |
| | ID NO: 886) | | | | | 1491) | | | | |
| AHH | DIVMTQSPKS | DIVM | ENVGT | VSWY | GAS | NRYT | GQSYS | CGQSY | WTFGGG | FGGG |
| 03744 | MSMSVGERVT | TQSP | Y (SEQ | QQKP | (SEQ ID | GVPD | YPWT | SYPWT | TKLEIK | TKLEI |
| | LSCKASENVG | KSMS | ID NO: | EQSP | NO: | RFTGS | (SEQ ID | F (SEQ | (SEQ ID | K |
| | TYVSWYQQK | MSVG | 933) | KLLIY | 975) | GSAT | NO: | ID NO: | NO: 1467) | (SEQ |
| | PEQSPKLLIYG | ERVT | | (SEQ | | DFTLT | 980) | 623) | | ID NO: |
| | ASNRYTGVPD | LSCK | | ID NO: | | ISSVQ | | | | 959) |
| | RFTGSGSATD | AS | | 1446) | | AEDL | | | | |
| | FTLTISSVQAE | (SEQ | | | | ADYH | | | | |
| | DLADYHCGQS | ID NO: | | | | C | | | | |
| | YSYPWTFGGG | 808) | | | | (SEQ | | | | |
| | TKLEIK (SEQ | | | | | ID NO: | | | | |
| | ID NO: 811) | | | | | 1162) | | | | |
| AHH | DIQMTQTPSSL | DIQM | QDIGS | LNWL | ATS | SLDS | LQYAS | CLQYA | TFGGGT | FGGG |
| 03745 | SASLGERVSLT | TQTPS | S (SEQ | QQEP | (SEQ ID | GVPK | SPRT | SSPRTF | KLEIK | TKLEI |
| | CRASQDIGSSL | SLSAS | ID NO: | DGTIK | NO: | RFSGS | (SEQ ID | (SEQ ID | (SEQ ID | K |
| | NWLQQEPDGT | LGER | 1199) | RLIY | 618) | RSGS | NO: | NO: | NO: 1393) | (SEQ |
| | IKRLIYATSSL | VSLT | | (SEQ | | DYSL | 1099) | 635) | | ID NO: |
| | DSGVPKRFSG | CRAS | | ID NO: | | TISSL | | | | 959) |
| | SRSGSDYSLTI | (SEQ | | 1085) | | ESEDF | | | | |
| | SSLESEDFVDY | ID NO: | | | | VDYY | | | | |
| | YCLQYASSPR | 753) | | | | C | | | | |
| | TFGGGTKLEIK | | | | | (SEQ | | | | |
| | (SEQ ID NO: | | | | | ID NO: | | | | |
| | 755) | | | | | 1366) | | | | |
| AHH | DIVMTQSPLSL | DIVM | QSLLY | LNWL | QVS | KLDP | LQGTY | CLQGT | WTFGGG | FGGG |
| 03746 | SVTIGQPASIS | TQSPL | SNGKT | QQRP | (SEQ ID | GIPDR | YPTWT | YYPTW | TKLEIK | TKLEI |
| | CKSSQSLLYS | SLSVT | Y (SEQ | GQAP | NO: | FSGSG | (SEQ ID | TF | (SEQ ID | K |
| | NGKTYLNWL | IGQPA | ID NO: | KHLMY | 1344) | SETDF | NO: | (SEQ ID | NO: 1467) | (SEQ |
| | QQRPGQAPKH | SISCK | 1303) | (SEQ | | TLKIS | 1092) | NO: | | ID NO: |
| | LMYQVSKLDP | SS | | ID NO: | | RVEA | | 629) | | 959) |
| | GIPDRFSGSGS | (SEQ | | 1086) | | EDLG | | | | |
| | ETDFTLKISRV | ID NO: | | | | VYYC | | | | |
| | EAEDLGVYYC | 812) | | | | (SEQ | | | | |
| | LQGTYYPTWT | | | | | ID NO: | | | | |
| | FGGGTKLEIK | | | | | 1053) | | | | |
| | (SEQ ID NO: | | | | | | | | | |
| | 813) | | | | | | | | | |
| AHH | DIVMSQSHKF | DIVM | QDVGT | VAWY | WAS | TRHT | QQYSS | CQQYS | TFGGGT | FGGG |
| 03747 | MSTSVGDRVS | SQSH | A (SEQ | QQKP | (SEQ ID | GVPD | YPLT | SYPLT | KLEIK | TKLEI |
| | ITCKASQDVG | KFMS | ID NO: | GQSP | NO: | RFTGS | (SEQ ID | F (SEQ | (SEQ ID | K |
| | TAVAWYQQK | TSVG | 1204) | KLLIY | 1450) | GSGT | NO: | ID NO: | NO: 1393) | (SEQ |
| | PGQSPKLLIY | DRVSI | | (SEQ | | DFTLT | 1291) | 673) | | ID NO: |
| | WASTRHTGVP | TCKA | | ID NO: | | ISIVQ | | | | 959) |
| | DRFTGSGSGT | S (SEQ | | 1427) | | SEDL | | | | |
| | DFTLTISIVQSE | ID NO: | | | | ADYF | | | | |
| | DLADYFCQQY | 779) | | | | C | | | | |
| | SSYPLTFGGGT | | | | | (SEQ | | | | |
| | KLEIK (SEQ ID | | | | | ID NO: | | | | |
| | NO: 780) | | | | | 1412) | | | | |
| AHH | DVVLTQTPLS | DVVL | QSLVH | LHWY | KVS | NRFS | SQSTH | CSQST | WTFGGG | FGGG |
| 03748 | LPVSLGDQASI | TQTPL | SNGNT | LQKP | (SEQ ID | GVPD | VPWT | HVPWT | TKLEMK | TKLE |
| | SCRSSQSLVHS | SLPVS | Y (SEQ | GQSP | NO: | RFSGS | (SEQ ID | F (SEQ | (SEQ ID | MK |
| | NGNTYLHWY | LGDQ | ID NO: | KLLIY | 1062) | GSGT | NO: | ID NO: | NO: 1468) | (SEQ |
| | LQKPGQSPKL | ASISC | 1306) | (SEQ | | DFTL | 1374) | 682) | | ID NO: |
| | LIYKVSNRFSG | RSS | | ID NO: | | KISRV | | | | 961) |
| | VPDRFSGSGS | (SEQ | | 1076) | | EAED | | | | |
| | GTDFTLKISRV | ID NO: | | | | LGVY | | | | |
| | EAEDLGVYFC | 874) | | | | FC | | | | |
| | SQSTHVPWTF | | | | | (SEQ | | | | |
| | GGGTKLEMK | | | | | ID NO: | | | | |
| | (SEQ ID NO: | | | | | 1157) | | | | |
| | 877) | | | | | | | | | |
| AHH | DIVMTQSHKF | DIVM | QDVST | VAWY | SAS | YRYT | QQHYS | CQQHY | TFGGGT | FGGG |
| 03749 | MSTSVGDRVS | TQSH | A (SEQ | QQKP | (SEQ ID | GVPD | TPPT | STPPTF | KLEIK | TKLEI |
| | ITCKASQDVST | KFMS | ID NO: | GQSP | NO: 13) | RFTGS | (SEQ ID | (SEQ ID | (SEQ ID | K |
| | AVAWYQQKP | TSVG | 1209) | KLLIH | | GSGT | NO: | NO: | NO: 1393) | (SEQ |
| | GQSPKLLIHSA | DRVSI | | (SEQ | | DFTFT | 1261) | 653) | | ID NO: |
| | SYRYTGVPDR | TCKA | | ID NO: | | ISSVQ | | | | 959) |
| | FTGSGSGTDFT | S (SEQ | | 1426) | | AEDL | | | | |
| | FTISSVQAEDL | ID NO: | | | | AVYY | | | | |
| | AVYYCQQHY | 800) | | | | C | | | | |
| | STPPTFGGGTK | | | | | (SEQ | | | | |
| | LEIK (SEQ ID | | | | | ID NO: | | | | |
| | NO: 801) | | | | | 1491) | | | | |
| AHH | DIVMTQSPLTL | DIVM | QSLLD | LNWL | LVS | KLDS | WQGT | CWQG | WTFGGG | FGGG |
| 03750 | SVTIGQPASIS | TQSPL | SDGKT | LQRP | (SEQ ID | GVPD | HFPWT | THFPW | TKLEIK | TKLEI |
| | CKSSQSLLDS | TLSVT | Y (SEQ | GQSP | NO: | RFTGS | (SEQ ID | TF | (SEQ ID | K |
| | DGKTYLNWL | IGQPA | ID NO: | KRLIY | 1116) | GSGT | NO: | (SEQ ID | NO: 1467) | (SEQ |
| | LQRPGQSPKR | SISCK | 1301) | (SEQ | | DFTL | 1465) | NO: | | ID NO: |
| | LIYLVSKLDSG | SS | | ID NO: | | KISRV | | 689) | | 959) |
| | VPDRFTGSGS | (SEQ | | 1083) | | EAED | | | | |
| | GTDFTLKISRV | ID NO: | | | | LGVY | | | | |
| | EAEDLGVYYC | 814) | | | | YC | | | | |
| | WQGTHFPWTF | | | | | (SEQ | | | | |
| | GGGTKLEIK | | | | | ID NO: | | | | |
| | (SEQ ID NO: | | | | | 1056) | | | | |
| | 815) | | | | | | | | | |
| AHH | DIQMTQTPLT | DIQM | QSLLD | LNWL | LVS | KLDS | WQGA | CWQG | WTFGGG | FGGG |
| 03751 | LSVTIGQPASI | TQTPL | SDGKT | LQRP | (SEQ ID | GVPD | HFPWT | AHFPW | TKLEIK | TKLEI |
| | SCKSSQSLLDS | TLSVT | Y (SEQ | GQSP | NO: | RFTGS | (SEQ ID | TF | (SEQ ID | K |
| | DGKTYLNWL | IGQPA | ID NO: | KRLIY | 1116) | GSGT | NO: | (SEQ ID | NO: 1467) | (SEQ |
| | LQRPGQSPKR | SISCK | 1301) | (SEQ | | DFTL | 1461) | NO: | | ID NO: |
| | LIYLVSKLDSG | SS | | ID NO: | | KISRV | | 685) | | 959) |
| | VPDRFTGSGS | (SEQ | | 1083) | | EAED | | | | |
| | GTDFTLKISRV | ID NO: | | | | LGVY | | | | |
| | EAEDLGVYYC | 748) | | | | YC | | | | |
| | WQGAHFPWT | | | | | (SEQ | | | | |
| | FGGGTKLEIK | | | | | ID NO: | | | | |
| | (SEQ ID NO: | | | | | 1056) | | | | |
| | 749) | | | | | | | | | |
| AHH | DIQMTQTPLT | DIQM | QSLLD | LNWL | LVS | KLDS | WQGT | CWQG | TFGGGT | FGGG |
| 03752 | LSVTIGQPASI | TQTPL | SDGKT | LQRP | (SEQ ID | GVPD | HFPQT | THFPQ | KLEMK | TKLE |
| | SCKSSQSLLDS | TLSVT | Y (SEQ | GQSP | NO: | RFTGS | (SEQ ID | TF | (SEQ ID | MK |
| | DGKTYLNWL | IGQPA | ID NO: | KRLIY | 1116) | GSGT | NO: | (SEQ ID | NO: 1394) | (SEQ |
| | LQRPGQSPKR | SISCK | 1301) | (SEQ | | DFTL | 1463) | NO: | | ID NO: |
| | LIYLVSKLDSG | SS | | ID NO: | | KISRV | | 687) | | 961) |
| | VPDRFTGSGS | (SEQ | | 1083) | | EAED | | | | |
| | GTDFTLKISRV | ID NO: | | | | LGVY | | | | |
| | EAEDLGVYYC | 748) | | | | YC | | | | |
| | WQGTHFPQTF | | | | | (SEQ | | | | |
| | GGGTKLEMK | | | | | ID NO: | | | | |
| | (SEQ ID NO: | | | | | 1056) | | | | |
| | 750) | | | | | | | | | |
| AHH | DIVITQSQKFM | DIVIT | QNVGS | VAWY | SAS | YRYS | QQYDS | CQQYD | YTFGGG | FGGG |
| 03753 | STSVGDRVSV | QSQK | N (SEQ | QQKP | (SEQ ID | GVPD | YPYT | SYPYT | TKLEIK | TKLEI |
| | TCKASQNVGS | FMST | ID NO: | GQSP | NO: 13) | RFTGS | (SEQ ID | F (SEQ | (SEQ ID | K |
| | NVAWYQQKP | SVGD | 1244) | KALIY | | GSGT | NO: | ID NO: | NO: 1493) | (SEQ |
| | GQSPKALIYSA | RVSV | | (SEQ | | DFTLT | 1280) | 669) | | ID NO: |
| | SYRYSGVPDR | TCKA | | ID NO: | | ISNVQ | | | | 959) |
| | FTGSGSGTDFT | S (SEQ | | 1425) | | SEDL | | | | |
| | LTISNVQSEDL | ID NO: | | | | AEYF | | | | |
| | AEYFCQQYDS | 766) | | | | C | | | | |
| | YPYTFGGGTK | | | | | (SEQ | | | | |
| | LEIK (SEQ ID | | | | | ID NO: | | | | |
| | NO: 767) | | | | | 1483) | | | | |
| AHH | DIQMTQSPSSL | DIQM | QDIGS | LNWL | ATS | SLDS | LQYAS | CLQYA | TFGGGT | FGGG |
| 03754 | SASLGERVSLT | TQSPS | S (SEQ | QQEP | (SEQ ID | GVPK | SPHT | SSPHTF | KLEIK | TKLEI |
| | CRASQDIGSSL | SLSAS | ID NO: | DGTIK | NO: | RFSGS | (SEQ ID | (SEQ ID | (SEQ ID | K |
| | NWLQQEPDGT | LGER | 1199) | RLIY | 618) | RSGS | NO: | NO: | NO: 1393) | (SEQ |
| | IKRLIYATSSL | VSLT | | (SEQ | | DYSL | 1098) | 634) | | ID NO: |
| | DSGVPKRFSG | CRAS | | ID NO: | | TISSL | | | | 959) |
| | SRSGSDYSLTI | (SEQ | | 1085) | | ESEDF | | | | |
| | SSLESEDFVDY | ID NO: | | | | VDYY | | | | |
| | YCLQYASSPH | 726) | | | | C | | | | |
| | TFGGGTKLEIK | | | | | (SEQ | | | | |
| | (SEQ ID NO: | | | | | ID NO: | | | | |
| | 727) | | | | | 1366) | | | | |
| AHH | DIQMTQSPAS | DIQM | ENIYS | LAWY | AAT | NLAD | QHFW | CQHF | WTFGGG | FGGG |
| 03755 | LSVSVGETVTI | TQSP | N (SEQ | QQKQ | (SEQ ID | GVPS | GTPWT | WGTP | TKLEMK | TKLE |
| | TCRASENIYSN | ASLS | ID NO: | GKSP | NO: | RFSGS | (SEQ ID | WTF | (SEQ ID | MK |
| | LAWYQQKQG | VSVG | 929) | QLLVY | 600) | GSGT | NO: | (SEQ ID | NO: 1468) | (SEQ |
| | KSPQLLVYAA | ETVTI | | (SEQ | | QYSL | 1213) | NO: | | ID NO: |
| | TNLADGVPSR | TCRA | | ID NO: | | KINSL | | 643) | | 961) |
| | FSGSGSGTQY | S (SEQ | | 1069) | | QSED | | | | |
| | SLKINSLQSED | ID NO: | | | | FGSY | | | | |
| | FGSYYCQHFW | 723) | | | | YC | | | | |
| | GTPWTFGGGT | | | | | (SEQ | | | | |
| | KLEMK (SEQ | | | | | ID NO: | | | | |
| | ID NO: 725) | | | | | 1143) | | | | |
| AHH | DTTVTQSHKF | DTTV | QDVST | VAWY | SAS | YRYT | QQHYS | CQQHY | YTFGGG | FGGG |
| 03756 | MSTSVGDRVS | TQSH | A (SEQ | QQKP | (SEQ ID | GVPD | TPYT | STPYT | TKLEIK | TKLEI |
| | ITCKASQDVST | KFMS | ID NO: | GQSP | NO: 13) | RFTGS | (SEQ ID | F (SEQ | (SEQ ID | K |
| | AVAWYQQKP | TSVG | 1209) | KLLIY | | GSGT | NO: | ID NO: | NO: 1493) | (SEQ |
| | GQSPKLLIYSA | DRVSI | | (SEQ | | DFTFT | 1265) | 657) | | ID NO: |
| | SYRYTGVPDR | TCKA | | ID NO: | | ISSVQ | | | | 959) |
| | FTGSGSGTDFT | S (SEQ | | 1427) | | AEDL | | | | |
| | FTISSVQAEDL | ID NO: | | | | AVYY | | | | |
| | AVYYCQQHY | 840) | | | | C | | | | |
| | STPYTFGGGT | | | | | (SEQ | | | | |
| | KLEIK (SEQ ID | | | | | ID NO: | | | | |
| | NO: 842) | | | | | 1491) | | | | |
| AHH | DIQMTQSPAS | DIQM | ENIYS | LAWY | AAT | NLAD | QHFW | CQHF | WTFGGG | FGGG |
| 03757 | LSVSVGETVAI | TQSP | N (SEQ | QQKQ | (SEQ ID | GVPS | GTPWT | WGTP | TKLEMK | TKLE |
| | TCRASENIYSN | ASLS | ID NO: | GKSP | NO: | RFSGS | (SEQ ID | WTF | (SEQ ID | MK |
| | LAWYQQKQG | VSVG | 929) | QLLVY | 600) | GSGT | NO: | (SEQ ID | NO: 1468) | (SEQ |
| | KSPQLLVYAA | ETVAI | | (SEQ | | QYSL | 1213) | NO: | | ID NO: |
| | TNLADGVPSR | TCRA | | ID NO: | | KINSL | | 643) | | 961) |
| | FSGSGSGTQY | S (SEQ | | 1069) | | QSED | | | | |
| | SLKINSLQSED | ID NO: | | | | FGSY | | | | |
| | FGSYYCQHFW | 721) | | | | YC | | | | |
| | GTPWTFGGGT | | | | | (SEQ | | | | |
| | KLEMK (SEQ | | | | | ID NO: | | | | |
| | ID NO: 722) | | | | | 1143) | | | | |

TABLE 3B
Exemplary Clones-Light Chain Sequences

| | | | | | | | | JUNC- | J- | |
|---|---|---|---|---|---|---|---|---|---|---|
| ID | V-J-REGION | L-FR1 | CDRL1 | L-FR2 | CDRL2 | L-FR3 | CDRL3 | TION | REGION | L-FR4 |
| AHH | DILMTQSPASL | DILM | TDIDD | MNWY | EGN | TLRP | LQSDN | CLQS | TFGGGT | FGGG |
| 03758 | SVATGEKVTI | TQSP | D (SEQ | QQKPG | (SEQ ID | GVPS | MPLT | DNMP | KLEIK | TKLEI |
| | RCITSTDIDDD | ASLS | ID NO: | EPPKL | NO: | RFSSS | (SEQ | LTF | (SEQ ID | K |
| | MNWYQQKPG | VATG | 1391) | LIS | 925) | GYGT | ID NO: | (SEQ | NO: 1393) | (SEQ |
| | EPPKLLISEGN | EKVTI | | (SEQ ID | | DFVF | 1094) | ID NO: | | ID NO: |
| | TLRPGVPSRFS | RCITS | | NO: | | TIENT | | 631) | | 959) |
| | SSGYGTDFVF | (SEQ | | 1134) | | LSED | | | | |
| | TIENTLSEDVA | ID NO: | | | | VADY | | | | |
| | DYYCLQSDN | 704) | | | | YC | | | | |
| | MPLTFGGGTK | | | | | (SEQ | | | | |
| | LEIK (SEQ ID | | | | | ID NO: | | | | |
| | NO: 705) | | | | | 1406) | | | | |
| AHH | DTTVTQSPAS | DTTV | QSVDY | MNWY | AAS | NLES | QQSNE | CQQS | YTFGGG | FGGG |
| 03759 | LAVSLGQRAT | TQSP | DGDSY | QQKPG | (SEQ ID | GIPAR | DPYT | NEDP | TKLEMK | TKLE |
| | ISCKASQSVD | ASLA | (SEQ ID | QPPKL | NO: | FSGSG | (SEQ | YTF | (SEQ ID | MK |
| | YDGDSYMNW | VSLG | NO: | LIY | 599) | SGTD | ID NO: | (SEQ | NO: 1494) | (SEQ |
| | YQQKPGQPPK | QRATI | 1308) | (SEQ ID | | FTLNI | 1270) | ID NO: | | ID NO: |
| | LLIYAASNLES | SCKA | | NO: | | HPVE | | 662) | | 961) |
| | GIPARFSGSGS | S (SEQ | | 1135) | | EEDA | | | | |
| | GTDFTLNIHPV | ID NO: | | | | ATYY | | | | |
| | EEEDAATYYC | 847) | | | | C | | | | |
| | QQSNEDPYTF | | | | | (SEQ | | | | |
| | GGGTKLEMK | | | | | ID NO: | | | | |
| | (SEQ ID NO: | | | | | 1148) | | | | |
| | 848) | | | | | | | | | |
| AHH | DVVMTQTPKF | DVVM | QSVSN | VAWY | YAS | NRYT | QQDY | CQQD | TFGGGT | FGGG |
| 03760 | LLVSAGDRVT | TQTP | D (SEQ | QQKPG | (SEQ ID | GVPD | SSPPT | YSSPP | KLEIK | TKLEI |
| | ITCKASQSVSN | KFLL | ID NO: | QSPKL | NO: | RFTGS | (SEQ | TF | (SEQ ID | K |
| | DVAWYQQKP | VSAG | 1310) | LIY | 1476) | GYGT | ID NO: | (SEQ | NO: 1393) | (SEQ |
| | GQSPKLLIYY | DRVTI | | (SEQ ID | | dftft | 1250) | ID NO: | | ID NO: |
| | ASNRYTGVPD | TCKA | | NO: | | ISTVQ | | 646) | | 959) |
| | RFTGSGYGTD | S (SEQ | | 1427) | | AEDL | | | | |
| | FTFTISTVQAE | ID NO: | | | | AVYF | | | | |
| | DLAVYFCQQD | 896) | | | | C | | | | |
| | YSSPPTFGGGT | | | | | (SEQ | | | | |
| | KLEIK (SEQ ID | | | | | ID NO: | | | | |
| | NO: 897) | | | | | 1164) | | | | |
| AHH | DVVLTQTPLS | DVVL | QTLLH | LKWY | QVS | NRFS | FQGSH | CFQG | WTFGGG | FGGG |
| 03761 | LPVTLGDPASI | TQTPL | SDGNT | LQKPG | (SEQ ID | GVPD | VPWT | SHVP | TKLEIK | TKLEI |
| | SCRSSQTLLHS | SLPVT | Y (SEQ | QSPKL | NO: | RFTGS | (SEQ | WTF | (SEQ ID | K |
| | DGNTYLKWY | LGDP | ID NO: | LIY | 1344) | GSGT | ID NO: | (SEQ | NO: 1467) | (SEQ |
| | LQKPGQSPKL | ASISC | 1311) | (SEQ ID | | DFTL | 969) | ID NO: | | ID NO: |
| | LIYQVSNRFSG | RSS | | NO: | | KISRV | | 620) | | 959) |
| | VPDRFTGSGS | (SEQ | | 1080) | | EAED | | | | |
| | GTDFTLKISRV | ID NO: | | | | LGVY | | | | |
| | EAEDLGVYHC | 883) | | | | HC | | | | |
| | FQGSHVPWTF | | | | | (SEQ | | | | |
| | GGGTKLEIK | | | | | ID NO: | | | | |
| | (SEQ ID NO: | | | | | 1160) | | | | |
| | 884) | | | | | | | | | |
| AHH | DIQMTQTPLT | DIQM | QSLLD | LNWLL | LVS | KLDS | WQGT | CWQG | TFGGGT | FGGG |
| 03763 | LSVTIGQPASI | TQTPL | SDGKT | QRPGQ | (SEQ ID | GVPD | HFPRT | THFPR | KLEIK | TKLEI |
| | SCKSSQSLLDS | TLSVT | Y (SEQ | SPKRLI | NO: | RFTGS | (SEQ | TF | (SEQ ID | K |
| | DGKTYLNWL | IGQPA | ID NO: | Y (SEQ | 1116) | GSGT | ID NO: | (SEQ | NO: 1393) | (SEQ |
| | LQRPGQSPKR | SISCK | 1301) | ID NO: | | DFTL | 1464) | ID NO: | | ID NO: |
| | LIYLVSKLDSG | SS | | 1083) | | KISRV | | 688) | | 959) |
| | VPDRFTGSGS | (SEQ | | | | EAED | | | | |
| | GTDFTLKISRV | ID NO: | | | | LGVY | | | | |
| | EAEDLGVYYC | 748) | | | | YC | | | | |
| | WQGTHFPRTF | | | | | (SEQ | | | | |
| | GGGTKLEIK | | | | | ID NO: | | | | |
| | (SEQ ID NO: | | | | | 1056) | | | | |
| | 751) | | | | | | | | | |
| AHH | DVVMTQSPAI | DVVM | ssvsss | LHWY | STS | NLAS | HQYH | CHQY | FGGGTK | FGGG |
| 03764 | MSASLGERVT | TQSP | Y (SEQ | QQKPG | (SEQ ID | GVPA | RSPPT | HRSPP | LEIK | TKLEI |
| | MTCTASSSVS | AIMS | ID NO: | SSPKL | NO: | RFSGS | (SEQ | TF | (SEQ ID | K |
| | SSYLHWYQQ | ASLG | 1384) | WIY | 1386) | GSGT | ID NO: | (SEQ | NO: 959) | (SEQ |
| | KPGSSPKLWI | ERVT | | (SEQ ID | | SYSLT | 1001) | ID NO: | | ID NO: |
| | YSTSNLASGV | MTCT | | NO: | | ISSME | | 628) | | 959) |
| | PARFSGSGSGT | AS | | 1077) | | AEDA | | | | |
| | SYSLTISSMEA | (SEQ | | | | ATYY | | | | |
| | EDAATYYCHQ | ID NO: | | | | C | | | | |
| | YHRSPPTFGG | 887) | | | | (SEQ | | | | |
| | GTKLEIK (SEQ | | | | | ID NO: | | | | |
| | ID NO: 888) | | | | | 1144) | | | | |
| AHH | DIVMTQSPAI | DIVM | SSVSY | MHWY | DTS | KLAS | QQWS | CQQW | YTFGGG | FGGG |
| 03765 | MSASPGEKVT | TQSP | (SEQ ID | QQKSG | (SEQ ID | GVPA | SNPPY | SSNPP | TKLEMK | TKLE |
| | MTCSASSSVS | AIMS | NO: | TSPKR | NO: | RFSGS | T (SEQ | YTF | (SEQ ID | MK |
| | YMHWYQQKS | ASPG | 1385) | WIY | 839) | GSGT | ID NO: | (SEQ | NO: 1494) | (SEQ |
| | GTSPKRWIYD | EKVT | | (SEQ ID | | SYSLT | 1275) | ID NO: | | ID NO: |
| | TSKLASGVPA | MTCS | | NO: | | ISSME | | 665) | | 961) |
| | RFSGSGSGTSY | AS | | 1131) | | AEDA | | | | |
| | SLTISSMEAED | (SEQ | | | | ATYY | | | | |
| | AATYYCQQW | ID NO: | | | | C | | | | |
| | SSNPPYTFGG | 802) | | | | (SEQ | | | | |
| | GTKLEMK | | | | | ID NO: | | | | |
| | (SEQ ID NO: | | | | | 1052) | | | | |
| | 804) | | | | | | | | | |
| AHH | DVVMTQTPLS | DVVM | QSLVH | LRWYL | KVS | NRFS | SQSTH | CSQST | WTFGGG | FGGG |
| 03766 | LPVSLGDQASI | TQTPL | SNGNT | QKPGQ | (SEQ ID | GVPD | VPWT | HVPW | TKLEIK | TKLEI |
| | SCRSSQSLVHS | SLPVS | Y (SEQ | SPKLLI | NO: | RFSGS | (SEQ | TF | (SEQ ID | K |
| | NGNTYLRWY | LGDQ | ID NO: | Y (SEQ | 1062) | GSGT | ID NO: | (SEQ | NO: 1467) | (SEQ |
| | LQKPGQSPKL | ASISC | 1306) | ID NO: | | DFTL | 1374) | ID NO: | | ID NO: |
| | LIYKVSNRFSG | RSS | | 1108) | | KISRV | | 682) | | 959) |
| | VPDRFSGSGS | (SEQ | | | | EAED | | | | |
| | GTDFTLKISRV | ID NO: | | | | LGVY | | | | |
| | EAEDLGVYFC | 900) | | | | FC | | | | |
| | SQSTHVPWTF | | | | | (SEQ | | | | |
| | GGGTKLEIK | | | | | ID NO: | | | | |
| | (SEQ ID NO: | | | | | 1157) | | | | |
| | 903) | | | | | | | | | |
| AHH | DVVMTQTPLT | DVVM | QSLLD | LNWLL | LVS | KLDS | WQGT | CWQG | TFGGGT | FGGG |
| 03767 | LSVTIGQPASI | TQTPL | SDGKT | QRPGQ | (SEQ ID | GVPD | HFPRT | THFPR | KLEIK | TKLEI |
| | SCKSSQSLLDS | TLSVT | Y (SEQ | SPKRLI | NO: | GFTG | (SEQ | TF | (SEQ ID | K |
| | DGKTYLNWL | IGQPA | ID NO: | Y (SEQ | 1116) | SGSG | ID NO: | (SEQ | NO: 1393) | (SEQ |
| | LQRPGQSPKR | SISCK | 1301) | ID NO: | | TDFTL | 1464) | ID NO: | | ID NO: |
| | LIYLVSKLDSG | SS | | 1083) | | KISRV | | 688) | | 959) |
| | VPDGFTGSGS | (SEQ | | | | EAED | | | | |
| | GTDFTLKISRV | ID NO: | | | | LGVY | | | | |
| | EAEDLGVYYC | 904) | | | | YC | | | | |
| | WQGTHFPRTF | | | | | (SEQ | | | | |
| | GGGTKLEIK | | | | | ID NO: | | | | |
| | (SEQ ID NO: | | | | | 1054) | | | | |
| | 908) | | | | | | | | | |
| AHH | DIQMTQTSSSF | DIQM | EDIYN | LAWY | GAT | SLETG | QQYW | CQQY | TFGGGT | FGGG |
| 03768 | SVSLGDRVTIT | TQTSS | R(SEQ | QQKPG | (SEQ ID | VPSRF | STPPT | WSTP | KLEIK | TKLEI |
| | CKASEDIYNR | SFSVS | ID NO: | NAPRL | NO: | SGSGS | (SEQ | PTF | (SEQ ID | K |
| | LAWYQQKPG | LGDR | 924) | LIS | 976) | GKDY | ID NO: | (SEQ | NO: 1393) | (SEQ |
| | NAPRLLISGAT | VTITC | | (SEQ ID | | TLSIT | 1292) | ID NO: | | ID NO: |
| | SLETGVPSRFS | KAS | | NO: | | SLQTE | | 674) | | 959) |
| | GSGSGKDYTL | (SEQ | | 1065) | | DVAT | | | | |
| | SITSLQTEDVA | ID NO: | | | | YYC | | | | |
| | TYYCQQYWS | 758) | | | | (SEQ | | | | |
| | TPPTFGGGTK | | | | | ID NO: | | | | |
| | LEIK (SEQ ID | | | | | 1367) | | | | |
| | NO: 759) | | | | | | | | | |
| AHH | DVVLTQTPLS | DVVL | QSLVH | LHWY | KVS | NRFS | SQSTH | CSQST | TFGGGT | FGGG |
| 03769 | LPVSLGDQASI | TQTPL | SNGNT | LQKPG | (SEQ ID | GVPD | VPRT | HVPR | KLEIK | TKLEI |
| | SCRSSQSLVHS | SLPVS | Y (SEQ | QSPKL | NO: | RFSGS | (SEQ | TF | (SEQ ID | K |
| | NGNTYLHWY | LGDQ | ID NO: | LIY | 1062) | GSGT | ID NO: | (SEQ | NO: 1393) | (SEQ |
| | LQKPGQSPKL | ASISC | 1306) | (SEQ ID | | DFTL | 1372) | ID NO: | | ID NO: |
| | LIYKVSNRFSG | RSS | | NO: | | KISRV | | 680) | | 959) |
| | VPDRFSGSGS | (SEQ | | 1076) | | EAED | | | | |
| | GTDFTLKISRV | ID NO: | | | | LGVY | | | | |
| | EAEDLGVYFC | 874) | | | | FC | | | | |
| | SQSTHVPRTF | | | | | (SEQ | | | | |
| | GGGTKLEIK | | | | | ID NO: | | | | |
| | (SEQ ID NO: | | | | | 1157) | | | | |
| | 878) | | | | | | | | | |
| AHH | DIVMSQSPSSL | DIVM | QSLLY | LAWY | WAS | TRES | QQYY | CQQY | FGGGTK | FGGG |
| 03770 | AVSVGEKVT | SQSPS | SSNQK | QQKPG | (SEQ ID | GVPD | SYP | YSYPF | LEIK | TKLEI |
| | MSCKSSQSLL | SLAV | NY | QSPKL | NO: | RFTGS | (SEQ | (SEQ | (SEQ ID | K |
| | YSSNQKNYLA | SVGE | (SEQ ID | LIY | 1450) | GSGT | ID NO: | ID NO: | NO: 959) | (SEQ |
| | WYQQKPGQSP | KVTM | NO: | (SEQ ID | | DFTLT | 1293) | 675) | | ID NO: |
| | KLLIYWASTR | SCKSS | 1304) | NO: | | ISSVK | | | | 959) |
| | ESGVPDRFTG | (SEQ | | 1066) | | AEDL | | | | |
| | SGSGTDFTLTI | ID NO: | | | | AVYY | | | | |
| | SSVKAEDLAV | 790) | | | | C | | | | |
| | YYCQQYYSYP | | | | | (SEQ | | | | |
| | FGGGTKLEIK | | | | | ID NO: | | | | |
| | (SEQ ID NO: | | | | | 1411) | | | | |
| | 791) | | | | | | | | | |
| AHH | DVVVTQTPLS | DVW | QSLVH | LHWY | KVS | NRFS | SQSTH | CSQST | YTFGGG | FGGG |
| 03771 | LPVSLGDQASI | TQTPL | SNGNT | LQKPG | (SEQ ID | GVPD | VYT | HVYT | TKLEMK | TKLE |
| | SCRSSQSLVHS | SLPVS | Y (SEQ | QSPKL | NO: | RFSGS | (SEQ | F (SEQ | (SEQ ID | MK |
| | NGNTYLHWY | LGDQ | ID NO: | LIY | 1062) | GSGT | ID NO: | ID NO: | NO: 1494) | (SEQ |
| | LQKPGQSPKL | ASISC | 1306) | (SEQ ID | | DFTL | 1376) | 684) | | ID NO: |
| | LIYKVSNRFSG | RSS | | NO: | | KISRV | | | | 961) |
| | VPDRFSGSGS | (SEQ | | 1076) | | DAED | | | | |
| | GTDFTLKISRV | ID NO: | | | | LGVY | | | | |
| | DAEDLGVYFC | 918) | | | | FC | | | | |
| | SQSTHVYTFG | | | | | (SEQ | | | | |
| | GGTKLEMK | | | | | ID NO: | | | | |
| | (SEQ ID NO: | | | | | 1155) | | | | |
| | 921) | | | | | | | | | |
| AHH | DIQMTQTPAS | DIQM | ENIYS | LAWY | NAK | TLAE | QHHY | CQHH | WTFGGG | FGGG |
| 03772 | LSASVGETVTI | TQTP | Y (SEQ | QQKQ | (SEQ ID | GVPS | GTPW | YGTP | TKLEIK | TKLEI |
| | TCRASENIYSY | ASLS | ID NO: | GKSPQ | NO: 28) | RFSGS | T (SEQ | WTF | (SEQ ID | K |
| | LAWYQQKQG | ASVG | 930) | LLVY | | GSGT | ID NO: | (SEQ | NO: 1467) | (SEQ |
| | KSPQLLVYNA | ETVTI | | (SEQ ID | | QFSL | 1217) | ID NO: | | ID NO: |
| | KTLAEGVPSR | TCRA | | NO: | | KINSL | | 644) | | 959) |
| | FSGSGSGTQFS | S (SEQ | | 1069) | | QPED | | | | |
| | LKINSLQPEDF | ID NO: | | | | FGSY | | | | |
| | GSYYCQHHY | 744) | | | | YC | | | | |
| | GTPWTFGGGT | | | | | (SEQ | | | | |
| | KLEIK (SEQ ID | | | | | ID NO: | | | | |
| | NO: 745) | | | | | 1398) | | | | |
| AHH | DVVLTQTPLS | DVVL | QSLVH | LHWY | KVS | NRFS | SQSTH | CSQST | TFGGGT | FGGG |
| 03773 | LPVSLGDQASI | TQTPL | SNGNT | LQKPG | (SEQ ID | GVPD | VPT | HVPT | KLEIK | TKLEI |
| | SCRSSQSLVHS | SLPVS | Y (SEQ | QSPKL | NO: | RFSGS | (SEQ | F (SEQ | (SEQ ID | K |
| | NGNTYLHWY | LGDQ | ID NO: | LIY | 1062) | GSGT | ID NO: | ID NO: | NO: 1393) | (SEQ |
| | LQKPGQSPKL | ASISC | 1306) | (SEQ ID | | DFTL | 1373) | 681) | | ID NO: |
| | LIYKVSNRFSG | RSS | | NO: | | KISRV | | | | 959) |
| | VPDRFSGSGS | (SEQ | | 1076) | | EAED | | | | |
| | GTDFTLKISRV | ID NO: | | | | LGVY | | | | |
| | EAEDLGVYFC | 874) | | | | FC | | | | |
| | SQSTHVPTFG | | | | | (SEQ | | | | |
| | GGTKLEIK | | | | | ID NO: | | | | |
| | (SEQ ID NO: | | | | | 1157) | | | | |
| | 879) | | | | | | | | | |
| AHH | DIVMSQSPAI | DIVM | SSVSY | MYWY | DTS | NLAS | QQWS | CQQW | SGGGTK | SGGG |
| 03774 | MSASPGEKVT | SQSPA | (SEQ ID | QQKPG | (SEQ ID | GVPV | SYPYT | SSYPY | LEIK | TKLEI |
| | MTRSASSSVS | IMSAS | NO: | SSPRLL | NO: | RFSGS | (SEQ | TS | (SEQ ID | K |
| | YMYWYQQKP | PGEK | 1385) | IY (SEQ | 839) | GSGT | ID NO: | (SEQ | NO: 1364) | (SEQ |
| | GSSPRLLIYDT | VTMT | | ID NO: | | SYSLT | 1278) | ID NO: | | ID NO: |
| | SNLASGVPVR | RSAS | | 1140) | | ISRME | | 667) | | 1364) |
| | FSGSGSGTSYS | (SEQ | | | | AEDA | | | | |
| | LTISRMEAED | ID NO: | | | | ATYY | | | | |
| | AATYYCQQW | 784) | | | | C | | | | |
| | SSYPYTSGGG | | | | | (SEQ | | | | |
| | TKLEIK (SEQ | | | | | ID NO: | | | | |
| | ID NO: 785) | | | | | 1146) | | | | |
| AHH | DIVMTQSPAL | DIVM | SSVSY | MYWY | LTP | NLAS | QQWS | CQQW | FGGGTK | FGGG |
| 03775 | MSASPGEKVT | TQSP | (SEQ ID | QQKPR | (SEQ ID | GVPA | SNPYT | SSNPY | LEIK | TKLEI |
| | MTCSASSSVS | ALMS | NO: | SSPKP | NO: | RFSGS | (SEQ | TF | (SEQ ID | K |
| | YMYWYQQKP | ASPG | 1385) | WIY | 1115) | GSGT | ID NO: | (SEQ | NO: 959) | (SEQ |
| | RSSPKPWIYLT | EKVT | | (SEQ ID | | SYSLT | 1276) | ID NO: | | ID NO: |
| | PNLASGVPAR | MTCS | | NO: | | ISSME | | 666) | | 959) |
| | FSGSGSGTSYS | AS | | 1141) | | AEDA | | | | |
| | LTISSMEAEDA | (SEQ | | | | ATYY | | | | |
| | ATYYCQQWSS | ID NO: | | | | C | | | | |
| | NPYTFGGGTK | 805) | | | | (SEQ | | | | |
| | LEIK (SEQ ID | | | | | ID NO: | | | | |
| | NO: 806) | | | | | 1144) | | | | |
| AHH | DIVMSQSPASL | DIVM | TDIDD | MNWY | EGN | TLRP | LQSDN | CLQS | YTFGGG | FGGG |
| 03776 | SVATGEKVTI | SQSPA | D (SEQ | QQKPG | (SEQ ID | GVPS | MPYT | DNMP | TKLEIK | TKLEI |
| | RCITSTDIDDD | SLSV | ID NO: | EPPKL | NO: | RFSSS | (SEQ | YTF | (SEQ ID | K |
| | MNWYQQKPG | ATGE | 1391) | LIS | 925) | GYGT | ID NO: | (SEQ | NO: 1493) | (SEQ |
| | EPPKLLISEGN | KVTIR | | (SEQ ID | | DFVF | 1095) | ID NO: | | ID NO: |
| | TLRPGVPSRFS | CITS | | NO: | | TIENT | | 632) | | 959) |
| | SSGYGTDFVF | (SEQ | | 1134) | | LSED | | | | |
| | TIENTLSEDVA | ID NO: | | | | VADY | | | | |
| | DYYCLQSDN | 788) | | | | YC | | | | |
| | MPYTFGGGTK | | | | | (SEQ | | | | |
| | LEIK (SEQ ID | | | | | ID NO: | | | | |
| | NO: 789) | | | | | 1406) | | | | |
| AHH | DIQMTQTPLT | DIQM | QSLLD | LNWLL | LVS | KLDS | WQGT | CWQG | VRRGDQ | FGGG |
| 03777 | LSVTIGQPASI | TQTPL | SDGKT | QRPGQ | (SEQ ID | GVPD | FSSH | TFSSH | AGNE | TKLE |
| | SCKSSQSLLDS | TLSVT | Y (SEQ | SPKRLI | NO: | RFTGS | (SEQ | V | (SEQ ID | MK |
| | DGKTYLNWL | IGQPA | ID NO: | Y (SEQ | 1116) | GSGT | ID NO: | (SEQ | NO: 1441) | (SEQ |
| | LQRPGQSPKR | SISCK | 1301) | ID NO: | | DFTL | 1462) | ID NO: | | ID NO: |
| | LIYLVSKLDSG | SS | | 1083) | | KISRV | | 686) | | 961) |
| | VPDRFTGSGS | (SEQ | | | | EAED | | | | |
| | GTDFTLKISRV | ID NO: | | | | LGVY | | | | |
| | EAEDLGVYYC | 748) | | | | YC | | | | |
| | WQGTFSSHVR | | | | | (SEQ | | | | |
| | RGDQAGNE | | | | | ID NO: | | | | |
| | (SEQ ID NO: | | | | | 1056) | | | | |
| | 752) | | | | | | | | | |
| AHH | DIVMSQSHKF | DIVM | QDVIT | VAWY | SAS | YRYT | QQHY | CQQH | TFGGGT | FGGG |
| 03778 | MSTSVGDRVS | SQSH | A (SEQ | QQKPG | (SEQ ID | GVPD | STPRT | YSTPR | KLEIK | TKLEI |
| | ITCKASQDVIT | KFMS | ID NO: | QSPKL | NO: 13) | RFTGS | (SEQ | TF | (SEQ ID | K |
| | AVAWYQQKP | TSVG | 1205) | PIY | | GSGT | ID NO: | (SEQ | NO: 1393) | (SEQ |
| | GQSPKLPIYSA | DRVSI | | (SEQ ID | | dftft | 1263) | ID NO: | | ID NO: |
| | SYRYTGVPDR | TCKA | | NO: | | ISSVQ | | 655) | | 959) |
| | FTGSGSGTDFT | S (SEQ | | 1428) | | AEDL | | | | |
| | FTISSVQAEDL | ID NO: | | | | AVYY | | | | |
| | AVYYCQQHY | 779) | | | | C | | | | |
| | STPRTFGGGT | | | | | (SEQ | | | | |
| | KLEIK (SEQ ID | | | | | ID NO: | | | | |
| | NO: 781) | | | | | 1491) | | | | |
| AHH | DIVMTQSPSPL | DIVM | QEISG | LSWLQ | AAS | TLDS | LQYAS | CLQY | TFGGGT | FGGG |
| 03779 | SASLGERVSLT | TQSPS | Y (SEQ | QKPDG | (SEQ ID | GVPK | YPRT | ASYP | KLEIK | TKLEI |
| | CRASQEISGYL | PLSAS | ID NO: | TIKRLI | NO: | RFSGS | (SEQ | RTF | (SEQ ID | K |
| | SWLQQKPDGT | LGER | 1210) | Y (SEQ | 599) | RSGS | ID NO: | (SEQ | NO: 1393) | (SEQ |
| | IKRLIYAASTL | VSLT | | ID NO: | | DYSL | 1102) | ID NO: | | ID NO: |
| | DSGVPKRFSG | CRAS | | 1111) | | TISSL | | 638) | | 959) |
| | SRSGSDYSLTI | (SEQ | | | | ESEDF | | | | |
| | SSLESEDFADY | ID NO: | | | | ADYY | | | | |
| | YCLQYASYPR | 817) | | | | C | | | | |
| | TFGGGTKLEIK | | | | | (SEQ | | | | |
| | (SEQ ID NO: | | | | | ID NO: | | | | |
| | 818) | | | | | 1402) | | | | |
| AHH | DTTVTQSPAS | DTTV | ESVDS | MHWY | RAS | NLES | QQSNE | CQQS | TFGGGT | FGGG |
| 03780 | LAVSLGQRAT | TQSP | YGNSF | QQKPG | (SEQ ID | GIPAR | DPRT | NEDP | KLEMK | TKLE |
| | ISCRASESVDS | ASLA | (SEQ ID | QPPKL | NO: | FSGSG | (SEQ | RTF | (SEQ ID | MK |
| | YGNSFMHWY | VSLG | NO: | LIY | 1346) | SRTGF | ID NO: | (SEQ | NO: 1394) | (SEQ |
| | QQKPGQPPKL | QRATI | 935) | (SEQ ID | | TLTIN | 1269) | ID NO: | | ID NO: |
| | LIYRASNLESG | SCRA | | NO: | | PVEA | | 661) | | 961) |
| | IPARFSGSGSR | S (SEQ | | 1129) | | DDVA | | | | |
| | TGFTLTINPVE | ID NO: | | | | TYYC | | | | |
| | ADDVATYYC | 850) | | | | (SEQ | | | | |
| | QQSNEDPRTF | | | | | ID NO: | | | | |
| | GGGTKLEMK | | | | | 1149) | | | | |
| | (SEQ ID NO: | | | | | | | | | |
| | 851) | | | | | | | | | |
| AHH | DILMTQSPATL | DILM | QSISD | LHWY | YAS | QSISG | QNGH | CQNG | TFGGGT | FGGG |
| 03781 | SVTPGDRVSL | TQSP | Y (SEQ | QQKSH | (SEQ ID | IPSRF | SFPRT | HSFPR | KLEIK | TKLEI |
| | SCRASQSISDY | ATLS | ID NO: | ESPRL | NO: | SGSGS | (SEQ | TF | (SEQ ID | K |
| | LHWYQQKSH | VTPG | 1296) | LIK | 1476) | GSDF | ID NO: | (SEQ | NO: 1393) | (SEQ |
| | ESPRLLIKYAS | DRVS | | (SEQ ID | | TLSIN | 1242) | ID NO: | | ID NO: |
| | QSISGIPSRFSG | LSCR | | NO: | | SVEPE | | 645) | | 959) |
| | SGSGSDFTLSI | AS | | 1079) | | DVGV | | | | |
| | NSVEPEDVGV | (SEQ | | | | YYC | | | | |
| | YYCQNGHSFP | ID NO: | | | | (SEQ | | | | |
| | RTFGGGTKLEI | 706) | | | | ID NO: | | | | |
| | K (SEQ ID NO: | | | | | 1297) | | | | |
| | 707) | | | | | | | | | |
| AHH | DVVMTQTPLS | DVVM | QSLVH | LHWY | KVS | NRFS | SQSTH | CSQST | WTFGGG | FGGG |
| 03782 | LPVSLGDQASI | TQTPL | SNGNT | LQKPG | (SEQ ID | GVPD | VPWT | HVPW | TKLEIK | TKLEI |
| | SCRSSQSLVHS | SLPVS | Y (SEQ | QSPKL | NO: | RFSGS | (SEQ | TF | (SEQ ID | K |
| | NGNTYLHWY | LGDQ | ID NO: | LIY | 1062) | GSGT | ID NO: | (SEQ | NO: 1467) | (SEQ |
| | LQKPGQSPKL | ASISC | 1306) | (SEQ ID | | DFTL | 1374) | ID NO: | | ID NO: |
| | LIYKVSNRFSG | RSS | | NO: | | KISRV | | 682) | | 959) |
| | VPDRFSGSGS | (SEQ | | 1076) | | EAED | | | | |
| | GTDFTLKISRV | ID NO: | | | | LGVY | | | | |
| | EAEDLGVYFC | 900) | | | | FC | | | | |
| | SQSTHVPWTF | | | | | (SEQ | | | | |
| | GGGTKLEIK | | | | | ID NO: | | | | |
| | (SEQ ID NO: | | | | | 1157) | | | | |
| | 901) | | | | | | | | | |
| AHH | DTTVTQSPAS | DTTV | QSVDY | MNWY | AAS | NLES | QQSNE | CQQS | YTFGGG | FGGG |
| 03783 | LAVSLGQRAT | TQSP | DGDSY | QQKPG | (SEQ ID | GIPAR | DPYT | NEDP | TKLEIK | TKLEI |
| | ISCKASQSVD | ASLA | (SEQ ID | QPPKL | NO: | FSGSG | (SEQ | YTF | (SEQ ID | K |
| | YDGDSYMNW | VSLG | NO: | LIY | 599) | SGTD | ID NO: | (SEQ | NO: 1493) | (SEQ |
| | YQQKPGQPPK | QRATI | 1308) | (SEQ ID | | FTLNI | 1270) | ID NO: | | ID NO: |
| | LLIYAASNLES | SCKA | | NO: | | HPVE | | 662) | | 959) |
| | GIPARFSGSGS | S (SEQ | | 1135) | | EEDA | | | | |
| | GTDFTLNIHPV | ID NO: | | | | ATYY | | | | |
| | EEEDAATYYC | 847) | | | | C | | | | |
| | QQSNEDPYTF | | | | | (SEQ | | | | |
| | GGGTKLEIK | | | | | ID NO: | | | | |
| | (SEQ ID NO: | | | | | 1148) | | | | |
| | 849) | | | | | | | | | |
| AHH | DTTVTQSPSSL | DTTV | QSLLN | LAWY | FAS | TRES | QQHY | CQQH | YTFGGG | FGGG |
| 03784 | AMSVGQKVT | TQSPS | SSNQK | QQKPG | (SEQ ID | GVPD | STPYT | YSTP | TKLEIK | TKLEI |
| | MSCKSSQSLL | SLAM | NY | QSPKL | NO: | RFIGS | (SEQ | YTF | (SEQ ID | K |
| | NSSNQKNYLA | SVGQ | (SEQ ID | LVY | 957) | GSGT | ID NO: | (SEQ | NO: 1493) | (SEQ |
| | WYQQKPGQSP | KVTM | NO: | (SEQ ID | | DFTLT | 1265) | ID NO: | | ID NO: |
| | KLLVYFASTR | SCKSS | 1302) | NO: | | ISSVQ | | 657) | | 959) |
| | ESGVPDRFIGS | (SEQ | | 1067) | | AEDL | | | | |
| | GSGTDFTLTIS | ID NO: | | | | ADYF | | | | |
| | SVQAEDLADY | 852) | | | | C | | | | |
| | FCQQHYSTPY | | | | | (SEQ | | | | |
| | TFGGGTKLEIK | | | | | ID NO: | | | | |
| | (SEQ ID NO: | | | | | 1410) | | | | |
| | 853) | | | | | | | | | |
| AHH | DIQMTQTHKF | DIQM | QDVST | VAWY | SAS | YRYT | QQHY | CQQH | VHVRRG | FGGG |
| 03785 | MSTSVGDRVS | TQTH | A (SEQ | QQKPG | (SEQ ID | GVPD | STHVH | YSTH | DQAGNQ | TKLEI |
| | ITCKASQDVST | KFMS | ID NO: | QSPKL | NO: 13) | RFTGS | (SEQ | VHV | (SEQ ID | K |
| | AVAWYQQKP | TSVG | 1209) | LIY | | GSGT | ID NO: | (SEQ | NO: 1434) | (SEQ |
| | GQSPKLLIYSA | DRVSI | | (SEQ ID | | dftft | 1259) | ID NO: | | ID NO: |
| | SYRYTGVPDR | TCKA | | NO: | | ISSVQ | | 652) | | 959) |
| | FTGSGSGTDFT | S (SEQ | | 1427) | | AEDL | | | | |
| | FTISSVQAEDL | ID NO: | | | | AVYY | | | | |
| | AVYYCQQHY | 741) | | | | C | | | | |
| | STHVHVRRGD | | | | | (SEQ | | | | |
| | QAGNQ (SEQ | | | | | ID NO: | | | | |
| | ID NO: 742) | | | | | 1491) | | | | |
| AHH | DVVVTQTPLS | DVW | QSLVH | LHWY | KVS | NRFS | SQSTH | CSQST | WTFGGG | FGGG |
| 03786 | LPVSLGDQASI | TQTPL | SNGNT | LQKPG | (SEQ ID | GVPD | VPWT | HVPW | TKLEIK | TKLEI |
| | SCRSSQSLVHS | SLPVS | Y (SEQ | QSPKL | NO: | RFSGS | (SEQ | TF | (SEQ ID | K |
| | NGNTYLHWY | LGDQ | ID NO: | LIY | 1062) | GSGT | ID NO: | (SEQ | NO: 1467) | (SEQ |
| | LQKPGQSPKL | ASISC | 1306) | (SEQ ID | | DFTL | 1374) | ID NO: | | ID NO: |
| | LIYKVSNRFSG | RSS | | NO: | | KISRV | | 682) | | 959) |
| | VPDRFSGSGS | (SEQ | | 1076) | | EAED | | | | |
| | GTDFTLKISRV | ID NO: | | | | LGVY | | | | |
| | EAEDLGVYFC | 918) | | | | FC | | | | |
| | SQSTHVPWTF | | | | | (SEQ | | | | |
| | GGGTKLEIK | | | | | ID NO: | | | | |
| | (SEQ ID NO: | | | | | 1157) | | | | |
| | 922) | | | | | | | | | |
| AHH | DILMTQSPAIM | DILM | SSVIY | MHWY | DTS | KLAS | QQWT | CQQW | FGGGTK | FGGG |
| 03787 | SASPGEKVTM | TQSP | (SEQ ID | QQKSG | (SEQ ID | GVPA | SNPPT | TSNPP | LEIK | TKLEI |
| | TCSASSSVIYM | AIMS | NO: | TSPKR | NO: | RFSGS | (SEQ | TF | (SEQ ID | K |
| | HWYQQKSGT | ASPG | 1383) | WIY | 839) | GSGT | ID NO: | (SEQ | NO: 959) | (SEQ |
| | SPKRWIYDTS | EKVT | | (SEQ ID | | SYSLS | 1279) | ID NO: | | ID NO: |
| | KLASGVPARF | MTCS | | NO: | | ISSME | | 668) | | 959) |
| | SGSGSGTSYSL | AS | | 1131) | | TEDA | | | | |
| | SISSMETEDAA | (SEQ | | | | ATYY | | | | |
| | TYYCQQWTS | ID NO: | | | | C | | | | |
| | NPPTFGGGTK | 702) | | | | (SEQ | | | | |
| | LEIK (SEQ ID | | | | | ID NO: | | | | |
| | NO: 703) | | | | | 1051) | | | | |
| AHH | DIVLTQSQKF | DIVLT | QNVGT | VAWY | SAS | YRYS | SQSTH | CSQST | YTFGGG | FGGG |
| 03788 | MSTSVGDRVS | QSQK | N (SEQ | QQKPG | (SEQ ID | GVPD | VPYT | HVPY | TKLEIK | TKLEI |
| | VTCKASQNVG | FMST | ID NO: | QSPKA | NO: 13) | RFTGS | (SEQ | TF | (SEQ ID | K |
| | TNVAWYQQK | SVGD | 12) | LIY | | GSGT | ID NO: | (SEQ | NO: 1493) | (SEQ |
| | PGQSPKALIYS | RVSV | | (SEQ ID | | DFTL | 1375) | ID NO: | | ID NO: |
| | ASYRYSGVPD | TCKA | | NO: | | KISRV | | 683) | | 959) |
| | RFTGSGSGTD | S (SEQ | | 1425) | | EAED | | | | |
| | FTLKISRVEAE | ID NO: | | | | LGVY | | | | |
| | DLGVYFCSQS | 773) | | | | FC | | | | |
| | THVPYTFGGG | | | | | (SEQ | | | | |
| | TKLEIK (SEQ | | | | | ID NO: | | | | |
| | ID NO: 776) | | | | | 1479) | | | | |
| AHH | DVVMTQTPLS | DVVM | QSLVH | LHWY | KVS | NRFS | SQSTH | CSQST | WTFGGG | FGGG |
| 03789 | LPVSLGDQASI | TQTPL | SNGNT | LQKPG | (SEQ ID | GVPD | VPWT | HVPW | TKLEIK | TKLEI |
| | SCRSSQSLVHS | SLPVS | Y (SEQ | QSPKL | NO: | RFSGS | (SEQ | TF | (SEQ ID | K |
| | NGNTYLHWY | LGDQ | ID NO: | LIY | 1062) | GSGT | ID NO: | (SEQ | NO: 1467) | (SEQ |
| | LQKPGQSPKL | ASISC | 1306) | (SEQ ID | | DFTL | 1374) | ID NO: | | ID NO: |
| | LIYKVSNRFSG | RSS | | NO: | | KISRV | | 682) | | 959) |
| | VPDRFSGSGS | (SEQ | | 1076) | | EAED | | | | |
| | GTDFTLKISRV | ID NO: | | | | LGVY | | | | |
| | EAEDLGVYFC | 900) | | | | FC | | | | |
| | SQSTHVPWTF | | | | | (SEQ | | | | |
| | GGGTKLEIK | | | | | ID NO: | | | | |
| | (SEQ ID NO: | | | | | 1157) | | | | |
| | 901) | | | | | | | | | |
| AHH | DIVMSQSPAIL | DIVM | SRVTY | MHWY | DTS | TLTSG | HQRS | CHQR | YTFGGG | FGGG |
| 03790 | SASPGEKVTM | SQSPA | (SEQ ID | QQKPG | (SEQ ID | VP AR | GYSYT | SGYS | TKLEMK | TKLE |
| | TCSASSRVTY | ILSAS | NO: | TSPKR | NO: | FSGSG | (SEQ | YTF | (SEQ ID | MK |
| | MHWYQQKPG | PGEK | 1380) | WIY | 839) | SGTS | ID NO: | (SEQ | NO: 1494) | (SEQ |
| | TSPKRWIYDT | VTMT | | (SEQ ID | | YSLTI | 1000) | ID NO: | | ID NO: |
| | STLTSGVPARF | CSAS | | NO: | | SGME | | 627) | | 961) |
| | SGSGSGTSYSL | (SEQ | | 1130) | | AEDA | | | | |
| | TISGMEAEDA | ID NO: | | | | ATYY | | | | |
| | ATYYCHQRSG | 782) | | | | C | | | | |
| | YSYTFGGGTK | | | | | (SEQ | | | | |
| | LEMK (SEQ ID | | | | | ID NO: | | | | |
| | NO: 783) | | | | | 1407) | | | | |
| AHH | DIVMSQSQKF | DIVM | QNVGT | VAWY | SAS | YRYS | QQYN | CQQY | TFGGGT | FGGG |
| 03791 | MSTSVGDRVS | SQSQ | N (SEQ | QQKPG | (SEQ ID | GVPD | SYPRT | NSYP | KLEIK | TKLEI |
| | VTCKASQNVG | KFMS | ID NO: | QSPKA | NO: 13) | RFTGS | (SEQ | RTF | (SEQ ID | K |
| | TNVAWYQQK | TSVG | 12) | LIY | | GSGT | ID NO: | (SEQ | NO: 1393) | (SEQ |
| | PGQSPKALIYS | DRVS | | (SEQ ID | | DFTLT | 1286) | ID NO: | | ID NO: |
| | ASYRYSGVPD | VTCK | | NO: | | ISNVQ | | 670) | | 959) |
| | RFTGSGSGTD | AS | | 1425) | | SEDL | | | | |
| | FTLTISNVQSE | (SEQ | | | | AEYF | | | | |
| | DLAEYFCQQY | ID NO: | | | | C | | | | |
| | NSYPRTFGGG | 793) | | | | (SEQ | | | | |
| | TKLEIK (SEQ | | | | | ID NO: | | | | |
| | ID NO: 794) | | | | | 1483) | | | | |
| AHH | DIVMTQAAPS | DIVM | KSLLH | LYWFL | RMS | NLAS | MQHL | CMQH | TFGGGT | FGGG |
| 03792 | VPVTPGESVSI | TQAA | SNGNT | QRPGQ | (SEQ ID | GVPD | EYPYT | LEYP | KLEIK | TKLEI |
| | SCRSSKSLLHS | PSVPV | Y (SEQ | SPQLLI | NO: | RFSGS | (SEQ | YTF | (SEQ ID | K |
| | NGNTYLYWFL | TPGES | ID NO: | Y (SEQ | 1360) | GSGT | ID NO: | (SEQ | NO: 1393) | (SEQ |
| | QRPGQSPQLLI | VSISC | 1060) | ID NO: | | AFTL | 1136) | ID NO: | | ID NO: |
| | YRMSNLASGV | RSS | | 1121) | | RISRV | | 641) | | 959) |
| | PDRFSGSGSGT | (SEQ | | | | EAED | | | | |
| | AFTLRISRVEA | ID NO: | | | | VGVY | | | | |
| | EDVGVYYCM | 796) | | | | YC | | | | |
| | QHLEYPYTFG | | | | | (SEQ | | | | |
| | GGTKLEIK | | | | | ID NO: | | | | |
| | (SEQ ID NO: | | | | | 1145) | | | | |
| | 797) | | | | | | | | | |
| AHH | DIVMTQSTSSL | DIVM | QDISN | LNWY | YTS | RLHS | QQGN | CQQG | WTFGGG | FGGG |
| 03794 | SASLGDRVTIS | TQSTS | Y (SEQ | QQKPD | (SEQ ID | GVPS | TLPWT | NTLP | TKLEIK | TKLEI |
| | CRASQDISNY | SLSAS | ID NO: | GTVKL | NO: | RFSGS | (SEQ | WTF | (SEQ ID | K |
| | LNWYQQKPD | LGDR | 27) | LIY | 1495) | GSGT | ID NO: | (SEQ | NO: 1467) | (SEQ |
| | GTVKLLIYYTS | VTISC | | (SEQ ID | | DYSL | 1255) | ID NO: | | ID NO: |
| | RLHSGVPSRFS | RAS | | NO: | | TISNL | | 649) | | 959) |
| | GSGSGTDYSL | (SEQ | | 1088) | | EQEDI | | | | |
| | TISNLEQEDIA | ID NO: | | | | ATYF | | | | |
| | TYFCQQGNTL | 828) | | | | C | | | | |
| | PWTFGGGTKL | | | | | (SEQ | | | | |
| | EIK (SEQ ID | | | | | ID NO: | | | | |
| | NO: 829) | | | | | 1353) | | | | |
| AHH | DIVMTQDELS | DIVM | KSLLY | LNCFL | LMS | TRAS | HQLV | CHQL | YTFGGG | FGGG |
| 03796 | NPVTSGESVSI | TQDE | KDGKT | QRPGQ | (SEQ ID | GVSN | EYPYT | VEYP | TKLEMK | TKLE |
| | SCRSSKSLLYK | LSNP | Y (SEQ | SPQLLI | NO: | RFSGS | (SEQ | YTF | (SEQ ID | MK |
| | DGKTYLNCFL | VTSG | ID NO: | Y (SEQ | 1081) | GSGT | ID NO: | (SEQ | NO: 1494) | (SEQ |
| | QRPGQSPQLLI | ESVSI | 1061) | ID NO: | | DFTLE | 998) | ID NO: | | ID NO: |
| | YLMSTRASGV | SCRSS | | 1082) | | ISRVK | | 625) | | 961) |
| | SNRFSGSGSGT | (SEQ | | | | AEDV | | | | |
| | DFTLEISRVKA | ID NO: | | | | GVYY | | | | |
| | EDVGVYYCH | 798) | | | | C | | | | |
| | QLVEYPYTFG | | | | | (SEQ | | | | |
| | GGTKLEMK | | | | | ID NO: | | | | |
| | (SEQ ID NO: | | | | | 1409) | | | | |
| | 799) | | | | | | | | | |
| AHH | DVVVTQTPLS | DVW | QSLEN | LNWY | RVS | NRFS | LQVT | CLQV | FAFGSGT | FGSG |
| 03797 | LPVSLGDQASI | TQTPL | SNGNT | LQKPG | (SEQ ID | GVLD | HVPFA | THVP | KLEIK | TKLEI |
| | SCRSSQSLENS | SLPVS | Y (SEQ | QSPQL | NO: | RFSGS | (SEQ | FAF | (SEQ ID | K |
| | NGNTYLNWY | LGDQ | ID NO: | LIY | 1361) | GSGT | ID NO: | (SEQ | NO: 956) | (SEQ |
| | LQKPGQSPQL | ASISC | 1300) | (SEQ ID | | DFTL | 1096) | ID NO: | | ID NO: |
| | LIYRVSNRFSG | RSS | | NO: | | KISRV | | 633) | | 965) |
| | VLDRFSGSGS | (SEQ | | 1087) | | EAED | | | | |
| | GTDFTLKISRV | ID NO: | | | | LGVY | | | | |
| | EAEDLGVYFC | 918) | | | | FC | | | | |
| | LQVTHVPFAF | | | | | (SEQ | | | | |
| | GSGTKLEIK | | | | | ID NO: | | | | |
| | (SEQ ID NO: | | | | | 1153) | | | | |
| | 919) | | | | | | | | | |
| AHH | DVVMTQTPLS | DVVM | QSLVH | LHWY | KVS | NRFS | SQSTH | CSQST | LTFGAG | FGAG |
| 03798 | LPVSLGDQASI | TQTPL | SNGNT | LQKPG | (SEQ ID | GVPD | VPLT | HVPL | TKLEIK | TKLEI |
| | SCRSSQSLVHS | SLPVS | Y (SEQ | QSPKL | NO: | RFSGS | (SEQ | TF | (SEQ ID | K |
| | NGNTYLHWY | LGDQ | ID NO: | LIY | 1062) | GSGT | ID NO: | (SEQ | NO: 1114) | (SEQ |
| | LQKPGQSPKL | ASISC | 1306) | (SEQ ID | | DFTL | 1370) | ID NO: | | ID NO: |
| | LIYKVSNRFSG | RSS | | NO: | | KISRV | | 679) | | 958) |
| | VPDRFSGSGS | (SEQ | | 1076) | | EAED | | | | |
| | GTDFTLKISRV | ID NO: | | | | LGVY | | | | |
| | EAEDLGVYFC | 900) | | | | FC | | | | |
| | SQSTHVPLTFG | | | | | (SEQ | | | | |
| | AGTKLEIK | | | | | ID NO: | | | | |
| | (SEQ ID NO: | | | | | 1157) | | | | |
| | 902) | | | | | | | | | |
| AHH | DVVLTQTPLS | DVVL | QSLVH | LHWY | KVS | NRFS | SQSTH | CSQST | TFGGGT | FGGG |
| 03799 | LPVSLGDQASI | TQTPL | SYGNT | LQKPG | (SEQ ID | GVPD | VPHT | HVPH | KLEMK | TKLE |
| | SCRSSQSLVHS | SLPVS | Y (SEQ | QSPKL | NO: | RFSGS | (SEQ | TF | (SEQ ID | MK |
| | YGNTYLHWY | LGDQ | ID NO: | LIY | 1062) | GSGT | ID NO: | (SEQ | NO: 1394) | (SEQ |
| | LQKPGQSPKL | ASISC | 1307) | (SEQ ID | | DFTL | 1369) | ID NO: | | ID NO: |
| | LIYKVSNRFSG | RSS | | NO: | | KISRV | | 678) | | 961) |
| | VPDRFSGSGS | (SEQ | | 1076) | | EAED | | | | |
| | GTDFTLKISRV | ID NO: | | | | LGVY | | | | |
| | EAEDLGVYFC | 874) | | | | FC | | | | |
| | SQSTHVPHTF | | | | | (SEQ | | | | |
| | GGGTKLEMK | | | | | ID NO: | | | | |
| | (SEQ ID NO: | | | | | 1157) | | | | |
| | 880) | | | | | | | | | |
| AHH | DIQMTQTPSSL | DIQM | QDIGS | LNWL | ATS | SLDS | LQYA | CLQY | YTFGGG | FGGG |
| 03800 | SASLGERVSLT | TQTPS | S (SEQ | QQEPD | (SEQ ID | GVPK | TFPYT | ATFP | TKLEIK | TKLEI |
| | CRASQDIGSSL | SLSAS | ID NO: | GTIKR | NO: | RFSGS | (SEQ | YTF | (SEQ ID | K |
| | NWLQQEPDGT | LGER | 1199) | LIS | 618) | RSGS | ID NO: | (SEQ | NO: 1493) | (SEQ |
| | IKRLISATSSL | VSLT | | (SEQ ID | | DYSL | 1104) | ID NO: | | ID NO: |
| | DSGVPKRFSG | CRAS | | NO: | | TISSL | | 639) | | 959) |
| | SRSGSDYSLTI | (SEQ | | 1084) | | ESEDF | | | | |
| | SSLESEDFVDY | ID NO: | | | | VDYY | | | | |
| | YCLQYATFPY | 753) | | | | C | | | | |
| | TFGGGTKLEIK | | | | | (SEQ | | | | |
| | (SEQ ID NO: | | | | | ID NO: | | | | |
| | 754) | | | | | 1366) | | | | |

TABLE 3C
Exemplary Clones-Light Chain Sequences

| | | | | | | | | JUNC- |
|---|---|---|---|---|---|---|---|---|
| ID | V-J-REGION | L-FR1 | CDRL1 | L-FR2 | CDRL2 | L-FR3 | CDRL3 | TION |
| >AH04501- | DVVMTQTQKFTSTSVG | DVVMT | KASQN | WYQQ | SASYR | VPDRF | QQYN | FGGGT |
| VL | DRVSVTCKASQNVGTN | QTQKFT | VGTNV | KPGQS | YSG | TGSGS | SYPYT | KLEIK |
| | VAWYQQKPGQSPKALI | STSVGD | A (SEQ | PKALI | (SEQ | GTDFT | (SEQ | (SEQ |
| | YSASYRYSGVPDRFTGS | RVSVTC | ID NO: | Y (SEQ | ID NO: | LTISN | ID NO: | ID NO: |
| | GSGTDFTLTISNVQSEDL | (SEQ ID | 1038) | ID NO: | 1362) | VQSED | 15) | 959) |
| | AEYFCQQYNSYPYTFGG | NO: 910) | | 1471) | | LAEYF | | |
| | GTKLEIK (SEQ ID NO: | | | | | C (SEQ | | |
| | 912) | | | | | ID NO: | | |
| | | | | | | 1439) | | |
| >AH04502- | DIVMTQSQKFMSTSVGD | DIVMTQ | KASQN | WYQQ | SASYR | VPDRF | QQYN | FGGGT |
| VL | RVSVTCKASQNVGINVA | SQKFMS | VGINV | KPGQS | YSG | TGSGS | SYPYT | KLEIK |
| | WYQQKPGQSPKALIYSA | TSVGDR | A (SEQ | PKALI | (SEQ | GTDFT | (SEQ | (SEQ |
| | SYRYSGVPDRFTGSGSG | VSVTC | ID NO: | Y (SEQ | ID NO: | LTISN | ID NO: | ID NO: |
| | TDFTLTISNVQSEDLAEY | (SEQ ID | 1037) | ID NO: | 1362) | VQSED | 15) | 959) |
| | FCQQYNSYPYTFGGGTK | NO: 819) | | 1471) | | LAEYF | | |
| | LEIK (SEQ ID NO: 822) | | | | | C (SEQ | | |
| | | | | | | ID NO: | | |
| | | | | | | 1439) | | |
| >AH04503- | DIQMTQSQKFMSTSVGD | DIQMTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPW | KLEIK |
| | AWYQQKPGQSPKALIYS | TSVGDR | A (SEQ | PKALI | (SEQ | GTDFT | T (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 36) | 959) |
| | YFCQQYNSYPWTFGGG | NO: 733) | | 1471) | | LAEYF | | |
| | TKLEIK (SEQ ID NO: 34) | | | | | C (SEQ | | |
| | | | | | | ID NO: | | |
| | | | | | | 1439) | | |
| >AH04504- | DTTVTQSQKFMSTSVGD | DTTVTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEIK |
| | AWYQQKPGQSPKALIYS | TSVGDR | A (SEQ | PKALI | (SEQ | GTDFT | (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 959) |
| | YFCQQYNSYPYTFGGGT | NO: 855) | | 1471) | | LAEYF | | |
| | KLEIK (SEQ ID NO: 859) | | | | | C (SEQ | | |
| | | | | | | ID NO: | | |
| | | | | | | 1439) | | |
| >AH04505- | DTTVTQSQKFMSTSVGD | DTTVTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEM |
| | AWYQQKPGQSPKALIYS | TSVGDR | A (SEQ | PKALI | (SEQ | GTDFT | (SEQ | K (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 961) |
| | YFCQQYNSYPYTFGGGT | NO: 855) | | 1471) | | LAEYF | | |
| | KLEMK (SEQ ID NO: 860) | | | | | C (SEQ | | |
| | | | | | | ID NO: | | |
| | | | | | | 1439) | | |
| >AH04506- | DIQMTQSQKFMSTSVGD | DIQMTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPW | KLEIK |
| | AWYQQKPGQSPKALIYS | TSVGDR | A (SEQ | PKALI | (SEQ | GTDFT | T (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 36) | 959) |
| | YFCQQYNSYPWTFGGG | NO: 733) | | 1471) | | LAEYF | | |
| | TKLEIK (SEQ ID NO: 34) | | | | | C (SEQ | | |
| | | | | | | ID NO: | | |
| | | | | | | 1439) | | |
| >AH04507- | DTTVTQSQKFMSTSVGD | DTTVTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEIK |
| | AWYQQKPGQSPKALIYS | TSVGDR | A (SEQ | PKALI | (SEQ | GTDFT | (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 959) |
| | YFCQQYNSYPYTFGGGT | NO: 855) | | 1471) | | LAEYF | | |
| | KLEIK (SEQ ID NO: 859) | | | | | C (SEQ | | |
| | | | | | | ID NO: | | |
| | | | | | | 1439) | | |
| >AH04508- | DIVLTQSQKFMSTSVGD | DIVLTQ | KASQN | WYQQ | SASYR | VPDRF | QQYN | FGGGT |
| VL | RVSVTCKASQNVGTNV | SQKFMS | VGTNV | KPGQS | YSG | AGSGS | SYPYT | KLEIK |
| | AWYQQKPGQSPKALIYS | TSVGDR | A (SEQ | PKALI | (SEQ | GTDFT | (SEQ | (SEQ |
| | ASYRYSGVPDRFAGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 959) |
| | YFCQQYNSYPYTFGGGT | NO: 772) | | 1471) | | LAEYF | | |
| | KLEIK (SEQ ID NO: 775) | | | | | C (SEQ | | |
| | | | | | | ID NO: | | |
| | | | | | | 1437) | | |
| >AH04509- | DTTVTQSQRFMSTSVGD | DTTVTQ | KASQN | WYQQ | SASYR | VPDRF | QQYN | FGGGT |
| VL | RVSVTCKASQNVGTNV | SQRFMS | VGTNV | KPGQS | YSG | TGSGS | NSPLT | KLEIK |
| | AWYQQKPGQSPKPLIYS | TSVGDR | A (SEQ | PKPLI | (SEQ | GTDFT | (SEQ | (SEQ |
| | ASYRYSGVPDRFTGSGS | VSVTC | ID NO: | Y (SEQ | ID NO: | LTINN | ID NO: | ID NO: |
| | GTDFTLTINNVQSEDLA | (SEQ ID | 1038) | ID NO: | 1362) | VQSED | 1282) | 959) |
| | EYFCQQYNNSPLTFGGG | NO: 862) | | 1473) | | LAEYF | | |
| | TKLEIK (SEQ ID NO: 864) | | | | | C (SEQ | | |
| | | | | | | ID NO: | | |
| | | | | | | 1438) | | |
| >AH04510- | DIVMTQSQKFMSTSVGD | DIVMTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEIK |
| | AWYQQKPGQSPKALIYS | TSVGDR | A (SEQ | PKALI | (SEQ | GTDFT | (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 959) |
| | YFCQQYNSYPYTFGGGT | NO: 819) | | 1471) | | LAEYF | | |
| | KLEIK (SEQ ID NO: 823) | | | | | C (SEQ | | |
| | | | | | | ID NO: | | |
| | | | | | | 1439) | | |
| >AH04511- | DIVMSQSQKFMSTSVGD | DIVMSQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEIK |
| | AWYQQKPGQSPKALIYS | TSVGDR | A (SEQ | PKALI | (SEQ | GTDFT | (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 959) |
| | YFCQQYNSYPYTFGGGT | NO: 792) | | 1471) | | LAEYF | | |
| | KLEIK (SEQ ID NO: 795) | | | | | C (SEQ | | |
| | | | | | | ID NO: | | |
| | | | | | | 1439) | | |
| >AH04512- | DIQMTQSQKFMSTSVGD | DIQMTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEM |
| | AWYQQKPGQSPKALIYS | TSVGDR | A (SEQ | PKALI | (SEQ | GTDFT | (SEQ | K (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 961) |
| | YFCQQYNSYPYTFGGGT | NO: 733) | | 1471) | | LAEYF | | |
| | KLEMK (SEQ ID NO: 735) | | | | | C (SEQ | | |
| | | | | | | ID NO: | | |
| | | | | | | 1439) | | |
| >AH04513- | DILMTQSQKFMSTSVGD | DILMTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEIK |
| | AWYQQKPGQSPKALIYS | TSVGDR | A (SEQ | PKALI | (SEQ | GTDFT | (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 959) |
| | YFCQQYNSYPYTFGGGT | NO: 710) | | 1471) | | LAEYF | | |
| | KLEIK (SEQ ID NO: 712) | | | | | C (SEQ | | |
| | | | | | | ID NO: | | |
| | | | | | | 1439) | | |
| >AH04514- | DIQMTQSQKFMSTSVGD | DIQMTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEIK |
| | AWYQQKPGQSPKALIYS | TSVGDR | A (SEQ | PKALI | (SEQ | GTDFT | (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 959) |
| | YFCQQYNSYPYTFGGGT | NO: 733) | | 1471) | | LAEYF | | |
| | KLEIK (SEQ ID NO: 736) | | | | | C (SEQ | | |
| | | | | | | ID NO: | | |
| | | | | | | 1439) | | |
| >AH04515- | DIVLTQSQKFMSTSVGD | DIVLTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEM |
| | AWYQQKPGQSPKALIYS | TSVGDR | A (SEQ | PKALI | (SEQ | GTDFT | (SEQ | K (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 961) |
| | YFCQQYNSYPYTFGGGT | NO: 772) | | 1471) | | LAEYF | | |
| | KLEMK (SEQ ID NO: 777) | | | | | C (SEQ | | |
| | | | | | | ID NO: | | |
| | | | | | | 1439) | | |
| >AH04516- | DTTVTQSQKFMSTSVGD | DTTVTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPW | KLEIK |
| | AWYQQKPGQSPKALIYS | TSVGDR | A (SEQ | PKALI | (SEQ | GTDFT | T (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 36) | 959) |
| | YFCQQYNSYPWTFGGG | NO: 855) | | 1471) | | LAEYF | | |
| | TKLEIK (SEQ ID NO: 861) | | | | | C (SEQ | | |
| | | | | | | ID NO: | | |
| | | | | | | 1439) | | |
| >AH04517- | DIQMTQSQKFMSTSVGD | DIQMTQ | KASQS | WYQQ | SASYR | VPDRF | QQYN | FGGGT |
| VL | RVSVTCKASQSVGTNVA | SQKFMS | VGTNV | KPGQS | YSG | TGSGS | NYPW | KLEIK |
| | WYQQKPGQSPKALIYSA | TSVGDR | A (SEQ | PKALI | (SEQ | GTDFT | T (SEQ | (SEQ |
| | SYRYSGVPDRFTGSGSG | VSVTC | ID NO: | Y (SEQ | ID NO: | LTISN | ID NO: | ID NO: |
| | TDFTLTISNVQSEDLAEY | (SEQ ID | 1040) | ID NO: | 1362) | VQSED | 1284) | 959) |
| | FCQQYNNYPWTFGGGT | NO: 733) | | 1471) | | LAEYF | | |
| | KLEIK (SEQ ID NO: 737) | | | | | C (SEQ | | |
| | | | | | | ID NO: | | |
| | | | | | | 1439) | | |
| >AH04518- | DILLTQSQKFMSTSVGD | DILLTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEIK |
| | AWYQQKPGQSPKALIYS | TSVGDR | A (SEQ | PKALI | (SEQ | GTDFT | (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 959) |
| | YFCQQYNSYPYTFGGGT | NO: 699) | | 1471) | | LAEYF | | |
| | KLEIK (SEQ ID NO: 701) | | | | | C (SEQ | | |
| | | | | | | ID NO: | | |
| | | | | | | 1439) | | |
| >AH04520- | DIQMTQSQKFMSTSVGD | DIQMTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEM |
| | AWYQQKPGQSPKALIYS | TSVGDR | A (SEQ | PKALI | (SEQ | GTDFT | (SEQ | K (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 961) |
| | YFCQQYNSYPYTFGGGT | NO: 733) | | 1471) | | LAEYF | | |
| | KLEMK (SEQ ID NO: 735) | | | | | C (SEQ | | |
| | | | | | | ID NO: | | |
| | | | | | | 1439) | | |
| >AH04521- | DTTVTQSQKFMSTSVGD | DTTVTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEIK |
| | AWYQQKPGQSPKALIYS | TSVGDR | A (SEQ | PKALI | (SEQ | GTDFT | (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 959) |
| | YFCQQYNSYPYTFGGGT | NO: 855) | | 1471) | | LAEYF | | |
| | KLEIK (SEQ ID NO: 859) | | | | | C (SEQ | | |
| | | | | | | ID NO: | | |
| | | | | | | 1439) | | |
| >AH04522- | DTTVTQSQKFMSTSVGD | DTTVTQ | KAGQ | WYQQ | SASYR | VPDRF | QQYN | FGGGT |
| VL | RVSVTCKAGQNVGTNV | SQKFMS | NVGTN | KPGQS | YSG | TGSGS | SYPYT | KLEIK |
| | AWYQQKPGQSPKALIYS | TSVGDR | VA | PKALI | (SEQ | GTDFT | (SEQ | (SEQ |
| | ASYRYSGVPDRFTGSGS | VSVTC | (SEQ ID | Y (SEQ | ID NO: | LTISN | ID NO: | ID NO: |
| | GTDFTLTISNVQSEDLAE | (SEQ ID | NO: | ID NO: | 1362) | VQSED | 15) | 959) |
| | YFCQQYNSYPYTFGGGT | NO: 855) | 1036) | 1471) | | LAEYF | | |
| | KLEIK (SEQ ID NO: 857) | | | | | C (SEQ | | |
| | | | | | | ID NO: | | |
| | | | | | | 1439) | | |
| >AH04523- | DIVLTQSQKFMSTSVGD | DIVLTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEIK |
| | AWYQQKPGQSPKALIYS | TSVGDR | A (SEQ | PKALI | (SEQ | GTDFT | (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 959) |
| | YFCQQYNSYPYTFGGGT | NO: 772) | | 1471) | | LAEYF | | |
| | KLEIK (SEQ ID NO: 778) | | | | | C (SEQ | | |
| | | | | | | ID NO: | | |
| | | | | | | 1439) | | |
| >AH04524- | DIVMTQSQKFMSTSVGD | DIVMTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEIK |
| | AWYQQKPGQSPKALIYS | TSVGDR | A (SEQ | PKALI | (SEQ | GTDFT | (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 959) |
| | YFCQQYNSYPYTFGGGT | NO: 819) | | 1471) | | LAEYF | | |
| | KLEIK (SEQ ID NO: 825) | | | | | C (SEQ | | |
| | | | | | | ID NO: | | |
| | | | | | | 1439) | | |
| >AH04525- | DNVLTQSQKFMSTSVGD | DNVLT | KASQN | WYQQ | SASYR | VPDRF | QQYN | FGGGT |
| VL | RVSVTCKASQNVGTNV | QSQKF | VGTNV | KPGQS | YSG | TGSGS | SYPYT | KLEM |
| | AWYQQKPGQSPKALIYS | MSTSV | A (SEQ | PKALI | (SEQ | GTDFT | (SEQ | K (SEQ |
| | ASYRYSGVPDRFTGSGS | GDRVS | ID NO: | Y (SEQ | ID NO: | LTISN | ID NO: | ID NO: |
| | GTDFTLTISNVQSEDLAE | VTC | 1038) | ID NO: | 1362) | VQSED | 15) | 961) |
| | YFCQQYNSYPYTFGGGT | (SEQ ID | | 1471) | | LAEYF | | |
| | KLEMK (SEQ ID NO: 838) | NO: 836) | | | | C (SEQ | | |
| | | | | | | ID NO: | | |
| | | | | | | 1439) | | |
| >AH04526- | DTTVTQSQKFMSTSVGD | DTTVTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEM |
| | AWYQQKPGQSPKALIYS | TSVGDR | A (SEQ | PKALI | (SEQ | GTDFT | (SEQ | K (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 961) |
| | YFCQQYNSYPYTFGGGT | NO: 855) | | 1471) | | LAEYF | | |
| | KLEMK (SEQ ID NO: 860) | | | | | C (SEQ | | |
| | | | | | | ID NO: | | |
| | | | | | | 1439) | | |
| >AH04527- | DVVMTQSQKFMSTSVG | DVVMT | KASQN | WYQQ | SASYR | VPDRF | QQYN | FGGGT |
| VL | DRVSVTCKASQNVGTN | QSQKF | VGTNV | KPGQS | YSG | TGSGS | SYPYT | KLEIK |
| | VAWYQQKPGQSPKALI | MSTSV | A (SEQ | PKALI | (SEQ | GTDFT | (SEQ | (SEQ |
| | YSASYRYSGVPDRFTGS | GDRVS | ID NO: | Y (SEQ | ID NO: | LTISN | ID NO: | ID NO: |
| | GSGTDFTLTISNVQSEDL | VTC | 1038) | ID NO: | 1362) | VQSED | 15) | 959) |
| | AEYFCQQYNSYPYTFGG | (SEQ ID | | 1471) | | LAEYF | | |
| | GTKLEIK (SEQ ID NO: | NO: 893) | | | | C (SEQ | | |
| | 895) | | | | | ID NO: | | |
| | | | | | | 1439) | | |
| >AH04528- | DIQMTQSQKFMSTSVGD | DIQMTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEIK |
| | AWYQQKPGQSPKALIYS | TSVGDR | A (SEQ | PKALI | (SEQ | GTDFT | (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 959) |
| | YFCQQYNSYPYTFGGGT | NO: 733) | | 1471) | | LAEYF | | |
| | KLEIK (SEQ ID NO: 736) | | | | | C (SEQ | | |
| | | | | | | ID NO: | | |
| | | | | | | 1439) | | |
| >AH04529- | DTTVTQSQKFMSTSVGD | DTTVTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPYT | KLEIK |
| | AWYQQKPGQSPKALIYS | TSVGDR | A (SEQ | PKALI | (SEQ | GTDFT | (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 15) | 959) |
| | YFCQQYNSYPYTFGGGT | NO: 855) | | 1471) | | LAEYF | | |
| | KLEIK (SEQ ID NO: 859) | | | | | C (SEQ | | |
| | | | | | | ID NO: | | |
| | | | | | | 1439) | | |
| >AH04530- | DTTVTQSQKFMSTSVGD | DTTVTQ | KASQN | WYQQ | SASYR | VPDRF | QQYN | FGGGT |
| VL | RVSVTCKASQNVGTNV | SQKFMS | VGTNV | KPGQS | YSG | TGSGS | SYPW | KLEIK |
| | AWYQQKPGQSPKALIYS | TSVGDR | A (SEQ | PKALI | (SEQ | GTDFT | T (SEQ | (SEQ |
| | ASYRYSGVPDRFTGSGS | VSVTC | ID NO: | Y (SEQ | ID NO: | LTISN | ID NO: | ID NO: |
| | GTDFTLTISNVQSEDLAE | (SEQ ID | 1038) | ID NO: | 1362) | VQSED | 36) | 959) |
| | YFCQQYNSYPWTFGGG | NO: 855) | | 1471) | | LAEYF | | |
| | TKLEIK (SEQ ID NO: 861) | | | | | C (SEQ | | |
| | | | | | | ID NO: | | |
| | | | | | | 1439) | | |

TABLE 4A
Exemplary Clones-Heavy Chain Sequences

| ID | HFR1 | CDRH1 | HFR2 | CDRH2 | HFR3 | CDRH3 | HFR4 |
|---|---|---|---|---|---|---|---|
| BL_592989- | QVQLQQSGAELVRP | GYTFT | IGWVK | IHPGG | NYNEKFKG | TSRNF | WGQ |
| 2015_G2_P8_G02 | GTSLKMSCKAA | NYW | QRPGH | GYT | KATLTADTS | AY | GTPV |
| | (SEQ ID NO: 1324) | (SEQ ID | GLEWI | (SEQ ID | SSTTYMQLS | (SEQ ID | TVSS |
| | | NO: 4) | GD | NO: | SLTSEDSAIY | NO: | (SEQ |
| | | | (SEQ ID | 1015) | YC (SEQ ID | 1419) | ID |
| | | | NO: | | NO: 1181) | | NO: |
| | | | 1010) | | | | 398) |
| BL_592989- | PGPTQQPGSELVRPG | GYTFT | MHWV | IYPGSG | NYDEKFKSK | TRSGV | VWG |
| 2016_H2_P8_H02 | ASVKLSCKAS (SEQ | SYW | KQRHG | ST (SEQ | GTLTVDTSS | EGLLH | AGTT |
| | ID NO: 1191) | (SEQ ID | QGLEW | ID NO: | STAYMHLSS | WYFD | VTVS |
| | | NO: | IGN | 251) | LASEDSAVY | (SEQ ID | S |
| | | 157) | (SEQ ID | | YC (SEQ ID | NO: | (SEQ |
| | | | NO: | | NO: 1166) | 1416) | ID |
| | | | 1123) | | | | NO: |
| | | | | | | | 1448) |
| BL_592989- | QVQLQQSGSELVRP | GYTFT | MHWV | IYPGSG | NYDEKFKSK | TRWIT | WGR |
| 2017_A3_P8_A03 | GASVKLSCKAS (SEQ | SYW | KQRHG | ST (SEQ | GTLTVDTSS | TDHYF | GTTL |
| | ID NO: 1342) | (SEQ ID | QGLEW | ID NO: | STAYMHLSS | DY | TVSS |
| | | NO: | IGN | 251) | LTSEDSAVY | (SEQ ID | (SEQ |
| | | 157) | (SEQ ID | | CC (SEQ ID | NO: | ID |
| | | | NO: | | NO: 1167) | 1417) | NO: |
| | | | 1123) | | | | 1455) |
| BL_592989- | QIQLQQPGAELVRPG | GYTFT | LGWV | IYPGG | NYNEKFKG | ARVSP | WGQ |
| 2018_B3_P8_B03 | TSVKISCKAS (SEQ | NYW | KQRPG | GYT | KATLTADTS | AS | GTTL |
| | ID NO: 1220) | (SEQ ID | HGLEW | (SEQ ID | SSTAYMQLN | (SEQ ID | TVSS |
| | | NO: 4) | IGD | NO: 6) | SLTSEDSAV | NO: | (SEQ |
| | | | (SEQ ID | | YFC (SEQ ID | 615) | ID |
| | | | NO: | | NO: 1176) | | NO: |
| | | | 1075) | | | | 425) |
| BL_592989- | QVQLQQPGAELVRP | GYTFT | LGWV | IYPGG | NYNEKFKG | ARVTP | WGQ |
| 2019_C3_P8_C03 | GTSVKISCKAS (SEQ | NYW | KQRPG | GYT | KATLTADTS | AS | GTTL |
| | ID NO: 1314) | (SEQ ID | HGLEW | (SEQ ID | SSTAYMQLS | (SEQ ID | TVSS |
| | | NO: 4) | IGD | NO: 6) | SLTSEDSAV | NO: 8) | (SEQ |
| | | | (SEQ ID | | YFC (SEQ ID | | ID |
| | | | NO: | | NO: 1178) | | NO: |
| | | | 1075) | | | | 425) |
| BL_592989- | QIQLQQSGAELVRPG | GYTFT | IGWVK | IHPGG | NYNEKFKG | TSRNF | WGQ |
| 2021_E3_P8_E03 | TSVKMSCKAA (SEQ | NYW | QRPGH | GYT | KATLTADTS | AY | GTPV |
| | ID NO: 1228) | (SEQ ID | GLEWI | (SEQ ID | SSTAYMQLS | (SEQ ID | TVSS |
| | | NO: 4) | GD | NO: | SLTSEDSAIY | NO: | (SEQ |
| | | | (SEQ ID | 1015) | YC (SEQ ID | 1419) | ID |
| | | | NO: | | NO: 1177) | | NO: |
| | | | 1010) | | | | 398) |
| BL_592989- | QVQLQQSGAELVRP | GYTFT | IGWVK | IHPGG | NYNEKFKG | TSRNF | WGQ |
| 2022_F3_P8_F03 | GTSVKMSCKAA | NYW | QRPGH | GYT | KATLTADTS | AY | GTLV |
| | (SEQ ID NO: 1326) | (SEQ ID | GLEWI | (SEQ ID | SSTAYMQLS | (SEQ ID | TVSA |
| | | NO: 4) | GD | NO: | SLTSEGSAIY | NO: | (SEQ |
| | | | (SEQ ID | 1015) | YC (SEQ ID | 1419) | ID |
| | | | NO: | | NO: 1179) | | NO: |
| | | | 1010) | | | | 421) |
| BL_592989- | PGPTQQPGSELVRPG | GYTFT | MHWV | IYPGSG | NYDEKFKSK | TRSGV | VWG |
| 2023_G3_P8_G03 | ASVKLSCKAS (SEQ | SYW | KQRHG | ST (SEQ | GTLTVDTSS | EGLLH | AGTT |
| | ID NO: 1191) | (SEQ ID | QGLEW | ID NO: | STAYMHLSS | WYFD | VTVS |
| | | NO: | IGN | 251) | LASEDSAVY | (SEQ ID | S |
| | | 157) | (SEQ ID | | YC (SEQ ID | NO: | (SEQ |
| | | | NO: | | NO: 1166) | 1416) | ID |
| | | | 1123) | | | | NO: |
| | | | | | | | 1448) |
| BL_592989- | QVQLQQSGAEVAKP | GYTST | MHWV | INPSTG | EYNQKFEDK | ARGGY | WGQ |
| 2024_H3_P8_H03 | GASVKMSCKAS | GYW | KQRPG | YT | ATLTADKSS | FDY | GTSL |
| | (SEQ ID NO: 1340) | (SEQ ID | QGLEW | (SEQ ID | NTAYMQLSS | (SEQ ID | TVSS |
| | | NO: | IGY | NO: | LTSEDSAVY | NO: | (SEQ |
| | | 995) | (SEQ ID | 230) | YC (SEQ ID | 606) | ID |
| | | | NO: | | NO: 954) | | NO: |
| | | | 190) | | | | 424) |
| BL_592989- | QVQLQQSGAELVRP | GYTFT | LGWIK | IYPGG | NYNEKFKG | ARVTP | WGQ |
| 2025_A4_P8_A04 | GTSVKISCKAS (SEQ | NYW | QRPGH | GYT | KATLTADTS | AS | GTSL |
| | ID NO: 1325) | (SEQ ID | GLEWI | (SEQ ID | SSTAYMQLS | (SEQ ID | TVSS |
| | | NO: 4) | GD | NO: 6) | SLTSEDSAV | NO: 8) | (SEQ |
| | | | (SEQ ID | | YFC (SEQ ID | | ID |
| | | | NO: | | NO: 1178) | | NO: |
| | | | 1074) | | | | 424) |
| BL_592989- | QAYLQQSGAELVRP | GYTFT | MHWV | ISTYSG | SYNQKFKG | ARGVT | WGQ |
| 2026_B4_P8_B04 | GVSVKISCKGS (SEQ | DYA | KQSHA | DV | KATMTVDK | FDS | GTTL |
| | ID NO: 1196) | (SEQ ID | KSLEW | (SEQ ID | SSSTAYMEL | (SEQ ID | TVSS |
| | | NO: 19) | IGV | NO: 46) | ARLTPEDSAI | NO: 48) | (SEQ |
| | | | (SEQ ID | | YYC (SEQ ID | | ID |
| | | | NO: | | NO: 1388) | | NO: |
| | | | 1126) | | | | 425) |
| BL_592989- | QVQLQQSGAELVRP | GYTFT | LGWV | IYPGG | NYNEKFKG | ARVTP | WGQ |
| 2027_C4_P8_C04 | GTSVKISCKAS (SEQ | NYW | KQRPG | GYT | KATLTADTS | AS | GTTV |
| | ID NO: 1325) | (SEQ ID | HGLEW | (SEQ ID | SSTAYMQLS | (SEQ ID | TVSS |
| | | NO: 4) | IGD | NO: 6) | SLTSEDSAV | NO: 8) | (SEQ |
| | | | (SEQ ID | | YFC (SEQ ID | | ID |
| | | | NO: | | NO: 1178) | | NO: |
| | | | 1075) | | | | 422) |
| BL_592989- | QVQLQQSGAELVRP | GYTFT | IGWIK | IHPGG | NYNEKFKG | TGRNF | WGQ |
| 2029_E4_P8_E04 | GTSVKMSCKAA | NYW | QRPGH | DYT | KATLTADTF | AY | GTLV |
| | (SEQ ID NO: 1326) | (SEQ ID | GLEWI | (SEQ ID | SSTAYMQLS | (SEQ ID | TVSS |
| | | NO: 4) | GD | NO: | SLTSEDSAIY | NO: | (SEQ |
| | | | (SEQ ID | 1013) | YC (SEQ ID | 1395) | ID |
| | | | NO: | | NO: 1171) | | NO: |
| | | | 1007) | | | | 413) |
| BL_592989- | QVQLQQPGAELVRP | GYTFT | IGWVK | IHPGG | NYNEKFKG | TSRNF | WGQ |
| 2030_F4_P8_F04 | GTSVKMSCKAA | NYW | QRPGH | GYT | KATLTADTS | AY | GTLV |
| | (SEQ ID NO: 1315) | (SEQ ID | GLEWI | (SEQ ID | SSTAYMQLS | (SEQ ID | TVSA |
| | | NO: 4) | GD | NO: | SLTSEDSAIY | NO: | (SEQ |
| | | | (SEQ ID | 1015) | YC (SEQ ID | 1419) | ID |
| | | | NO: | | NO: 1177) | | NO: |
| | | | 1010) | | | | 421) |
| BL_592989- | QIQLQQSGAELVRPG | GYTFT | IGWVK | IHPGG | NYNEKFKG | TSRNF | WGQ |
| 2031_G4_P8_G04 | TSLKMSCKAA (SEQ | NYW | QRPGH | GYT | KATLTADTS | AY | GTLV |
| | ID NO: 1226) | (SEQ ID | GLEWI | (SEQ ID | SSTTYMQLS | (SEQ ID | TVSA |
| | | NO: 4) | GD | NO: | SLTSEDSAIY | NO: | (SEQ |
| | | | (SEQ ID | 1015) | YC (SEQ ID | 1419) | ID |
| | | | NO: | | NO: 1181) | | NO: |
| | | | 1010) | | | | 421) |
| BL_592989- | QVQLQQSGAELVRP | GYTFS | IGWVK | IHPGG | NYNEKFTGK | VSRNF | WGQ |
| 2032_H4_P8_H04 | GTSVKMSCKAA | NYW | QRPGH | GYI | ATLTADTSS | AN | GTLV |
| | (SEQ ID NO: 1326) | (SEQ ID | GLEWI | (SEQ ID | STAYMQLSS | (SEQ ID | TVSA |
| | | NO: | GD | NO: | LTSEDSAIY | NO: | (SEQ |
| | | 986) | (SEQ ID | 1014) | YC (SEQ ID | 1445) | ID |
| | | | NO: | | NO: 1184) | | NO: |
| | | | 1010) | | | | 421) |
| BL_592989- | QVQLQQSGAELVRP | GYTFT | MHWV | ISTYSG | SYNQKFKG | ARGVT | WGQ |
| 2033_A5_P8_A05 | GVSVKISCKGS (SEQ | DYA | KQSHA | DV | KATMTVDK | FDS | GTTL |
| | ID NO: 1338) | (SEQ ID | KSLEW | (SEQ ID | SSSTAYMEL | (SEQ ID | TVSS |
| | | NO: 19) | IGV | NO: 46) | ARLTSEDSAI | NO: 48) | (SEQ |
| | | | (SEQ ID | | YYC (SEQ ID | | ID |
| | | | NO: | | NO: 1389) | | NO: |
| | | | 1126) | | | | 425) |
| BL_592989- | QVQLQQSGAELVRP | GYTFT | LGWIK | IYPGG | NYNEKFKG | ARVTP | WGQ |
| 2034_B5_P8_B05 | GTSVKISCKAS (SEQ | NYW | QRPGH | GYT | KATLTADTS | AS | GTSL |
| | ID NO: 1325) | (SEQ ID | GLEWI | (SEQ ID | SSTAYMQLS | (SEQ ID | TVSS |
| | | NO: 4) | GD | NO: 6) | SLTSEDSAV | NO: 8) | (SEQ |
| | | | (SEQ ID | | YFC (SEQ ID | | ID |
| | | | NO: | | NO: 1178) | | NO: |
| | | | 1074) | | | | 424) |
| BL_592989- | QVQLQQPGAELVRP | GYTFT | LGWV | IYPGG | NYNEKFKG | ARVTP | WGQ |
| 2035_C5_P8_C05 | GTSVKISCKAS (SEQ | NYW | KQRPG | GYT | KATLTADTS | AS | GTSL |
| | ID NO: 1314) | (SEQ ID | HGLEW | (SEQ ID | SSTAYMQLS | (SEQ ID | TVSS |
| | | NO: 4) | IGD | NO: 6) | SLTSEDSAV | NO: 8) | (SEQ |
| | | | (SEQ ID | | YFC (SEQ ID | | ID |
| | | | NO: | | NO: 1178) | | NO: |
| | | | 1075) | | | | 424) |
| BL_592989- | QIQLQQSGAELVRPG | GYTFT | IGWVK | IHPGG | NYNEKFKG | TSRNF | WGQ |
| 2037_E5_P8_E05 | TSVKMSCKAA (SEQ | NYW | QRPGH | DYS | KATLTADTS | AY | GTLV |
| | ID NO: 1228) | (SEQ ID | GLEWI | (SEQ ID | SSTAYMNLS | (SEQ ID | TVSA |
| | | NO: 4) | GD | NO: | SLTSEDSAIY | NO: | (SEQ |
| | | | (SEQ ID | 1012) | YC (SEQ ID | 1419) | ID |
| | | | NO: | | NO: 1174) | | NO: |
| | | | 1010) | | | | 421) |
| BL_592989- | QVQLQQPGAELVRP | GYTFT | IGWVK | IHPGG | NYNEKFTGK | VSRNF | WGQ |
| 2038_F5_P8_F05 | GTSVKMSCKAA | NYW | QRPGH | GYI | ATLTADTSS | AN | GTLV |
| | (SEQ ID NO: 1315) | (SEQ ID | GLEWI | (SEQ ID | STAYMQLSS | (SEQ ID | TVSA |
| | | NO: 4) | GD | NO: | LTSEDSAIY | NO: | (SEQ |
| | | | (SEQ ID | 1014) | YC (SEQ ID | 1445) | ID |
| | | | NO: | | NO: 1184) | | NO: |
| | | | 1010) | | | | 421) |
| BL_592989- | QIQLQQSGAELVRPG | GYTFT | IGWVK | IHPGG | NYNEKFKG | TSRNF | WGQ |
| 2039_G5_P8_G05 | TSVKMSCKAA (SEQ | NYW | QRPGH | GYT | KATLTADTS | AY | GTLV |
| | ID NO: 1228) | (SEQ ID | GLEWI | (SEQ ID | SSTAYMQLS | (SEQ ID | TVSA |
| | | NO: 4) | GD | NO: | SLTSEDSAIY | NO: | (SEQ |
| | | | (SEQ ID | 1015) | YC (SEQ ID | 1419) | ID |
| | | | NO: | | NO: 1177) | | NO: |
| | | | 1010) | | | | 421) |
| BL_592989- | QIQLQQSGAELVRPG | GYTFT | IGWVK | IHPGG | NYNEKFKG | TSRNF | WGQ |
| 2040_H5_P8_H05 | TSVKMSCKAA (SEQ | NYW | QRPGH | GYT | KATLTADTS | AY | GTLV |
| | ID NO: 1228) | (SEQ ID | GLEWV | (SEQ ID | SSTAYMQLS | (SEQ ID | TVSA |
| | | NO: 4) | GD | NO: | SLTSEDSAIY | NO: | (SEQ |
| | | | (SEQ ID | 1015) | YC (SEQ ID | 1419) | ID |
| | | | NO: | | NO: 1177) | | NO: |
| | | | 1011) | | | | 421) |
| BL_592989- | QVQLQQSGAELVRP | GYTFT | MHWV | ISTYSG | IYNQKFKGK | ARGVT | WGQ |
| 2041_A6_P8_A06 | GVSLKISCKGS (SEQ | DYA | RQSHA | DA | ATMTVDKSS | FDY | GTTV |
| | ID NO: 1337) | (SEQ ID | KSLEW | (SEQ ID | STAYLELAR | (SEQ ID | TVSS |
| | | NO: 19) | IGV | NO: 21) | LTSDDSAIY | NO: 23) | (SEQ |
| | | | (SEQ ID | | YC (SEQ ID | | ID |
| | | | NO: | | NO: 1033) | | NO: |
| | | | 1128) | | | | 422) |
| BL_592989- | QIQLQQSGAEMVRP | GYTFT | MHWV | ISTYSG | SYNQKFKG | ARGVT | WGQ |
| 2042_B6_P8_B06 | GVSVKISCKGS (SEQ | DYA | RQSHA | DV | KATMTVDK | FDS | GTTL |
| | ID NO: 1241) | (SEQ ID | KSLEW | (SEQ ID | SSSTAYMEL | (SEQ ID | TVSS |
| | | NO: 19) | IGV | NO: 46) | ARLTSEDSAI | NO: 48) | (SEQ |
| | | | (SEQ ID | | YYC (SEQ ID | | ID |
| | | | NO: | | NO: 1389) | | NO: |
| | | | 1128) | | | | 425) |
| BL_592989- | QIQLQQSGAEMVRP | GYTFT | MHWV | ISTYSG | SYNQKFKG | ARGVT | WGQ |
| 2043_C6_P8_C06 | GVSVKISCKGS (SEQ | DYA | RQSHA | DV | KATMTVDK | FDS | GTTL |
| | ID NO: 1241) | (SEQ ID | KSLEW | (SEQ ID | SSSTAYMEL | (SEQ ID | TVSS |
| | | NO: 19) | IGV | NO: 46) | ARLTSEDSAI | NO: 48) | (SEQ |
| | | | (SEQ ID | | YYC (SEQ ID | | ID |
| | | | NO: | | NO: 1389) | | NO: |
| | | | 1128) | | | | 425) |
| BL_592989- | QVQLQQSGAELVRP | GYTFT | LGWV | IYPGG | NYNEKFKG | ARVTP | WGQ |
| 2044_D6_P8_D06 | GTSVKISCKAS (SEQ | NYW | KQRPG | GYT | KATLTADTS | AS | GTTL |
| | ID NO: 1325) | (SEQ ID | HGLEW | (SEQ ID | SSTAYMQLS | (SEQ ID | TVSS |
| | | NO: 4) | IGD | NO: 6) | SLTSEDSAV | NO: 8) | (SEQ |
| | | | (SEQ ID | | YFC (SEQ ID | | ID |
| | | | NO: | | NO: 1178) | | NO: |
| | | | 1075) | | | | 425) |
| BL_592989- | QIQLQQSGAELVRPG | GYTFT | IGWVK | IHPGG | NYNEKFKG | TSRNF | WGQ |
| 2045_E6_P8_E06 | TSVKMSCKAA (SEQ | NYW | QRPGH | DYS | KATLTADTS | AY | GTLV |
| | ID NO: 1228) | (SEQ ID | GLEWI | (SEQ ID | SSTAYMNLS | (SEQ ID | TVSA |
| | | NO: 4) | GD | NO: | SLTSEDSAIY | NO: | (SEQ |
| | | | (SEQ ID | 1012) | YC (SEQ ID | 1419) | ID |
| | | | NO: | | NO: 1174) | | NO: |
| | | | 1010) | | | | 421) |
| BL_592989- | QIQLQQSGAELVRPG | GYTFT | IGWVK | IHPGG | NYNEKFKG | TSRNF | WGQ |
| 2046_F6_P8_F06 | TSVKMSCKAA (SEQ | NYW | QRPGH | DYS | KATLTADTS | AY | GTLV |
| | ID NO: 1228) | (SEQ ID | GLEWI | (SEQ ID | SSTAYMNLS | (SEQ ID | TVSA |
| | | NO: 4) | GD | NO: | SLTSEDSAIY | NO: | (SEQ |
| | | | (SEQ ID | 1012) | YC (SEQ ID | 1419) | ID |
| | | | NO: | | NO: 1174) | | NO: |
| | | | 1010) | | | | 421) |
| BL_592989- | QIQLQQSGAELVRPG | GYTFT | IGWVK | IHPGG | NYNEKFKG | TSRNF | WGQ |
| 2047_G6_P8_G06 | TSVKMSCKAA (SEQ | NYW | QRPGH | GYT | KATLTADTS | AY | GTLV |
| | ID NO: 1228) | (SEQ ID | GLEWI | (SEQ ID | SSTAYMQLS | (SEQ ID | TVSA |
| | | NO: 4) | GD | NO: | SLTSEDSAIY | NO: | (SEQ |
| | | | (SEQ ID | 1015) | YC (SEQ ID | 1419) | ID |
| | | | NO: | | NO: 1177) | | NO: |
| | | | 1010) | | | | 421) |
| BL_592989- | QIQLQQSGAELVRPG | GYTFT | IGWVK | IHPGG | NYNEKFKG | TSRNF | WGQ |
| 2048_H6_P8_H06 | TSVKMSCKAA (SEQ | NYW | QRPGH | DYS | KATLTADTS | AY | GTLV |
| | ID NO: 1228) | (SEQ ID | GLEWI | (SEQ ID | SSTAYMNLS | (SEQ ID | TVSA |
| | | NO: 4) | GD | NO: | SLTSEDSAIY | NO: | (SEQ |
| | | | (SEQ ID | 1012) | YC (SEQ ID | 1419) | ID |
| | | | NO: | | NO: 1174) | | NO: |
| | | | 1010) | | | | 421) |
| BL_592989- | QIQLQQSGAEMVRP | GYTFT | MHWV | ISTYSG | SYNQKFKG | ARGVT | WGQ |
| 2049_A7_P8_A07 | GVSVKISCKGS (SEQ | DYA | RQSHA | DV | KATMTVDK | FDS | GTTL |
| | ID NO: 1241) | (SEQ ID | KSLEW | (SEQ ID | SSSTAYMEL | (SEQ ID | TVSS |
| | | NO: 19) | IGV | NO: 46) | ARLTSEDSAI | NO: 48) | (SEQ |
| | | | (SEQ ID | | YYC (SEQ ID | | ID |
| | | | NO: | | NO: 1389) | | NO: |
| | | | 1128) | | | | 425) |
| BL_592989- | QVQLQQSGAELVRP | GYTFT | LGWIK | IYPGG | NYNEKFKG | ARVTP | WGQ |
| 2051_C7_P8_C07 | GTSVKISCKAS (SEQ | NYW | QRPGH | GYT | KATLTADTS | AS | GTSL |
| | ID NO: 1325) | (SEQ ID | GLEWI | (SEQ ID | SSTAYMQLS | (SEQ ID | TVSS |
| | | NO: 4) | GD | NO: 6) | SLTSEDSAV | NO: 8) | (SEQ |
| | | | (SEQ ID | | YFC (SEQ ID | | ID |
| | | | NO: | | NO: 1178) | | NO: |
| | | | 1074) | | | | 424) |
| BL_592989- | QAYLQQSGAELVRS | GFNIK | IHWVK | IDPDN | EYAPKFQGK | TVFWY | WGQ |
| 2052_D7_P8_D07 | GASVKLSCTAS (SEQ | DYY | QRPEQ | GET | ATMTADTSS | GNNYA | GTLV |
| | ID NO: 1197) | (SEQ ID | GLEWI | (SEQ ID | NTAHLQLSS | GFAY | TVSA |
| | | NO: | GW | NO: | LTSEDTAVY | (SEQ ID | (SEQ |
| | | 977) | (SEQ ID | 1003) | YC (SEQ ID | NO: | ID |
| | | | NO: | | NO: 946) | 1420) | NO: |
| | | | 1017) | | | | 421) |
| BL_592989- | QVQLQQSGAELVRP | GYTFT | IGWVK | IHPGG | NYNEKFKG | TSRNF | WGQ |
| 2054_F7_P8_F07 | GTSVKMSCKAA | NYW | QRPGH | DYS | KATLTADTS | AY | GTLV |
| | (SEQ ID NO: 1326) | (SEQ ID | GLEWI | (SEQ ID | SSTAYMNLS | (SEQ ID | TVSA |
| | | NO: 4) | GD | NO: | SLTSEDSAIY | NO: | (SEQ |
| | | | (SEQ ID | 1012) | YC (SEQ ID | 1419) | ID |
| | | | NO: | | NO: 1174) | | NO: |
| | | | 1010) | | | | 421) |
| BL_592989- | QIQLQQSGAELVRPG | GYTFT | IGWVK | INPGG | NYNEKFEGK | TSRNF | WGQ |
| 2055_G7_P8_G07 | TSVKMSCKAA (SEQ | NYW | LRPGH | GYT | ATLTADTSS | AY | GTLV |
| | ID NO: 1228) | (SEQ ID | GLEWI | (SEQ ID | STAYMQLSS | (SEQ ID | TVSA |
| | | NO: 4) | GD | NO: | LTSEDSAIY | NO: | (SEQ |
| | | | (SEQ ID | 1023) | YC (SEQ ID | 1419) | ID |
| | | | NO: | | NO: 1168) | | NO: |
| | | | 1008) | | | | 421) |
| BL_592989- | QIQLQQSGAELVRPG | GYTFT | LGWV | IYPGG | NYNEKFKG | VRVTP | WGQ |
| 2059_C8_P8_C08 | TSVKISCKAS (SEQ | NYW | KQRPG | GYT | KATLTADTS | AS | GTTV |
| | ID NO: 1227) | (SEQ ID | HGLEW | (SEQ ID | PSTAYMQLS | (SEQ ID | TVSS |
| | | NO: 4) | IGD | NO: 6) | SLTSEDSAV | NO: | (SEQ |
| | | | (SEQ ID | | YFC (SEQ ID | 1443) | ID |
| | | | NO: | | NO: 1172) | | NO: |
| | | | 1075) | | | | 422) |
| BL_592989- | QIQLQQSGAELVRPG | GYTFT | MQWV | ISTYSG | IYNQKFKGK | ARGVT | WGQ |
| 2060_D8_P8_D08 | VSLKISCKGS (SEQ | DYA | RQSHA | DA | ATMTVDKSS | FDY | GTTL |
| | ID NO: 1239) | (SEQ ID | KSLEW | (SEQ ID | STAYLELAR | (SEQ ID | TVSS |
| | | NO: 19) | IGV | NO: 21) | LTSEDSAIY | NO: 23) | (SEQ |
| | | | (SEQ ID | | YC (SEQ ID | | ID |
| | | | NO: | | NO: 1034) | | NO: |
| | | | 1137) | | | | 425) |
| BL_592989- | QVQLQQSGAELVRP | GYTFT | IGWVK | IHPGG | NYNEKFTGK | VSRNF | WGQ |
| 2061_E8_P8_E08 | GTSVKMSCKAA | NYW | QRPGH | GYI | ATLTADTSS | AN | GTPV |
| | (SEQ ID NO: 1326) | (SEQ ID | GLEWI | (SEQ ID | STAYMQLSS | (SEQ ID | TVSS |
| | | NO: 4) | GD | NO: | LTSEDSAIY | NO: | (SEQ |
| | | | (SEQ ID | 1014) | YC (SEQ ID | 1445) | ID |
| | | | NO: | | NO: 1184) | | NO: |
| | | | 1010) | | | | 398) |
| BL_592989- | QVQLQQSGAELVRP | GYTFT | MHWV | IITYSG | LYNQKFKG | AXGVT | WGQ |
| 2062_F8_P8_F08 | GVSLKISCKGS (SEQ | DYA | RQSHA | DA | KATMTVDK | FDY | GTSL |
| | ID NO: 1337) | (SEQ ID | KNLEW | (SEQ ID | SSSTAYLEL | (SEQ ID | TVSS |
| | | NO: 19) | IGV | NO: | ARLTSDDSA | NO: | (SEQ |
| | | | (SEQ ID | 1020) | IYYC (SEQ | 619) | ID |
| | | | NO: | | ID NO: | | NO: |
| | | | 1127) | | 1119) | | 424) |
| BL_592989- | PIQLQQSGAELVRPG | GYTFT | MNWV | IDPYDS | HYNQKFKD | AREAS | YWG |
| 2063_G8_P8_G08 | ASVKLSCKAS (SEQ | SYW | KQRPE | ET (SEQ | KAILTVDKS | YYYGN | QGTL |
| | ID NO: 1192) | (SEQ ID | QGLEW | ID NO: | SSTAYMQLS | AWFA | VTAL |
| | | NO: | IGR | 235) | SLTSEDSAV | (SEQ ID | S |
| | | 157) | (SEQ ID | | YYC | NO: | (SEQ |
| | | | NO: | | (SEQ ID | 601) | ID |
| | | | 195) | | NO: 269) | | NO: |
| | | | | | | | 1496) |
| BL_592989- | QIQLQQSGAELVRPG | GYTST | IGWVK | IHPGG | NYNEKFKG | TSRNF | WGQ |
| 2064_H8_P8_H08 | TSVKMSCKAA (SEQ | NYW | QRPGH | DYS | KATLTADTS | AY | GTLV |
| | ID NO: 1228) | (SEQ ID | GLEWI | (SEQ ID | SSTAYMNLS | (SEQ ID | TVSA |
| | | NO: | GD | NO: | SLTSEDSAIY | NO: | (SEQ |
| | | 997) | (SEQ ID | 1012) | YC (SEQ ID | 1419) | ID |
| | | | NO: | | NO: 1174) | | NO: |
| | | | 1010) | | | | 421) |
| BL_592989- | QVQLQQSGAELVRP | gytft | MHWV | ISTYSG | LYNQKFKG | ARGVT | WGQ |
| 2065_A9_P8_A09 | GVSLKISCKGS (SEQ | DYA | RQSHA | DA | KATMTVDK | FDY | GTSL |
| | ID NO: 1337) | (SEQ ID | KSLEW | (SEQ ID | SSSTAYLEL | (SEQ ID | TVSS |
| | | NO: 19) | IGV | NO: 21) | ARLTSDDSA | NO: 23) | (SEQ |
| | | | (SEQ ID | | IYYC (SEQ | | ID |
| | | | NO: | | ID NO: | | NO: |
| | | | 1128) | | 1119) | | 424) |
| BL_592989- | QIQLQQPGAELVRPG | GYAFT | LGWV | IYPGG | NYNEKFKG | ARVTP | WGQ |
| 2067_C9_P8_C09 | TSVKISCKAS (SEQ | NYW | KQRPG | GYT | KATLTADTS | AS | GTTV |
| | ID NO: 1220) | (SEQ ID | HGLEW | (SEQ ID | SSTAYMQLS | (SEQ ID | TVSS |
| | | NO: | IGD | NO: 6) | SLTSEDSAV | NO: 8) | (SEQ |
| | | 984) | (SEQ ID | | YFC (SEQ ID | | ID |
| | | | NO: | | NO: 1178) | | NO: |
| | | | 1075) | | | | 422) |
| BL_592989- | QVQLQQSGAELVRP | GYTFT | MHWV | ISTYSG | IYNQKFKGK | ARGVT | WGQ |
| 2068_D9_P8_D09 | GVSLKISCKGS (SEQ | DYA | RQSHA | DA | ATMTVDKSS | FDY | GTTV |
| | ID NO: 1337) | (SEQ ID | KSLEW | (SEQ ID | STAYLELAR | (SEQ ID | TVSS |
| | | NO: 19) | IGV | NO: 21) | LTSDDSAIY | NO: 23) | (SEQ |
| | | | (SEQ ID | | YC (SEQ ID | | ID |
| | | | NO: | | NO: 1033) | | NO: |
| | | | 1128) | | | | 422) |
| BL_592989- | QAYLQQSGAELVRP | GYTFT | IGWVK | IHPGG | NYNEKFKG | TSRNF | WGQ |
| 2069_E9_P8_E09 | GTSVKMSCKAA | NYW | QRPGH | DYS | KATLTADTS | AY | GTLV |
| | (SEQ ID NO: 1194) | (SEQ ID | GLEWI | (SEQ ID | SSTAYMNLS | (SEQ ID | TVSA |
| | | NO: 4) | GD | NO: | SLTSEDSAIY | NO: | (SEQ |
| | | | (SEQ ID | 1012) | YC (SEQ ID | 1419) | ID |
| | | | NO: | | NO: 1174) | | NO: |
| | | | 1010) | | | | 421) |
| BL_592989- | QIQLQQSGAELVRPG | GYTFT | MHWV | ISTYSG | SYNQKFKG | ARGVT | WGQ |
| 2070_F9_P8_F09 | VSVKISCKGS (SEQ | DYA | KQSHA | DV | KATMTVDK | FDS | GTSL |
| | ID NO: 1240) | (SEQ ID | KSLEW | (SEQ ID | SSSTAYMEL | (SEQ ID | TVSS |
| | | NO: 19) | IGV | NO: 46) | ARLTSEDSAI | NO: 48) | (SEQ |
| | | | (SEQ ID | | YYC (SEQ ID | | ID |
| | | | NO: | | NO: 1389) | | NO: |
| | | | 1126) | | | | 424) |
| BL_592989- | QVQLQQSGAELVRP | GYTFT | IGWVK | IHPGG | NYNEKFKG | TSRNF | WGQ |
| 2071_G9_P8_G09 | GTSVRMSCKAA | NYW | QRPGH | GYT | KATLTADTS | AY | GTLV |
| | (SEQ ID NO: 1336) | (SEQ ID | GLEWI | (SEQ ID | SSTAYMQLS | (SEQ ID | TVSA |
| | | NO: 4) | GD | NO: | SLTSEDSAIY | NO: | (SEQ |
| | | | (SEQ ID | 1015) | YC (SEQ ID | 1419) | ID |
| | | | NO: | | NO: 1177) | | NO: |
| | | | 1010) | | | | 421) |
| BL_592989- | QVQLQQSGAELVRP | GYTFT | LGWV | IYPGG | NYNEKFKG | ARVTP | WGQ |
| 207_4B_10P_8B10 | GTSVKISCKAS (SEQ | NYW | KQRPG | GYT | KATLTADTS | AS | GTTL |
| | ID NO: 1325) | (SEQ ID | HGLEW | (SEQ ID | SSTAYMQLS | (SEQ ID | TVSS |
| | | NO: 4) | IGD | NO: 6) | SLTSEDSAV | NO: 8) | (SEQ |
| | | | (SEQ ID | | YFC (SEQ ID | | ID |
| | | | NO: | | NO: 1178) | | NO: |
| | | | 1075) | | | | 425) |
| BL_592989- | QIQLQQPGAELVRPG | GYTFT | LGWV | IYPGG | NYNEKFKG | ARVTP | WGQ |
| 2076_D10_P8_D10 | TSVKISCKAS (SEQ | NYW | KQRPG | GYT | KATLTADTS | AS | GTSL |
| | ID NO: 1220) | (SEQ ID | HGLEW | (SEQ ID | SSTAYMQLS | (SEQ ID | TVSS |
| | | NO: 4) | IGD | NO: 6) | SLTSEDSAV | NO: 8) | (SEQ |
| | | | (SEQ ID | | YFC (SEQ ID | | ID |
| | | | NO: | | NO: 1178) | | NO: |
| | | | 1075) | | | | 424) |
| BL_592989- | QVQLQQPGAELVRP | GYTFT | IGWVK | IHPGG | NYNEKFEGK | TSRNF | WGQ |
| 2077_E10_P8_E10 | GTSVKMSCKAA | NYW | QRPGH | GYT | ATLTADTSS | AY | GTLV |
| | (SEQ ID NO: 1315) | (SEQ ID | GLEWI | (SEQ ID | STAYMQLSS | (SEQ ID | TVSA |
| | | NO: 4) | GD | NO: | LTSEGSAIY | NO: | (SEQ |
| | | | (SEQ ID | 1015) | YC (SEQ ID | 1419) | ID |
| | | | NO: | | NO: 1169) | | NO: |
| | | | 1010) | | | | 421) |
| BL_592989- | QIQLQQSGAELVRPG | GYTFT | IGWVK | IHPGG | NYNEKFKG | TSRNF | WGQ |
| 2078_F10_P8_F10 | TSVKMSCKAA (SEQ | NYW | QRPGH | DYS | KATLTADTS | AY | GTLV |
| | ID NO: 1228) | (SEQ ID | GLEWI | (SEQ ID | SSTAYMNLS | (SEQ ID | TVSA |
| | | NO: 4) | GD | NO: | SLTSGDSAIY | NO: | (SEQ |
| | | | (SEQ ID | 1012) | YC (SEQ ID | 1419) | ID |
| | | | NO: | | NO: 1175) | | NO: |
| | | | 1010) | | | | 421) |
| BL_592989- | QVQLQQSGAELVRP | GYTFT | IGWVK | FYPGG | NYNEKFKG | TSRNF | WGQ |
| 2079_G10_P8_G10 | GTSVKMSCKAA | NYW | QRPGH | DYI | KATLTADTS | AY | GTLV |
| | (SEQ ID NO: 1326) | (SEQ ID | GLEWI | (SEQ ID | SSTAYMQLS | (SEQ ID | TVSA |
| | | NO: 4) | GD | NO: | SLTSEDSAIY | NO: | (SEQ |
| | | | (SEQ ID | 973) | YC (SEQ ID | 1419) | ID |
| | | | NO: | | NO: 1177) | | NO: |
| | | | 1010) | | | | 421) |
| BL_592989- | QIQLQQSGAELAKPG | GYTFT | MHWV | INPSTG | EYNEKFEDK | ARWG | WGQ |
| 2080_H10_P8_H10 | ASVKMSCKAS (SEQ | RYW | KQRPG | YT | ATLTADKSS | NFDY | GTSL |
| | ID NO: 1225) | (SEQ ID | QGLEW | (SEQ ID | STAYMQLSS | (SEQ ID | TVSS |
| | | NO: | IGY | NO: | LTSEDSAVY | NO: | (SEQ |
| | | 992) | (SEQ ID | 230) | YC (SEQ ID | 617) | ID |
| | | | NO: | | NO: 950) | | NO: |
| | | | 190) | | | | 424) |
| BP003-T2P1A7 | QIQLQQSGAEMVRP | GYTFT | MHWV | ISTYSG | SYNQKFKG | ARGVT | WGQ |
| | GVSVKISCKGS (SEQ | DYA | RQSHA | DV | KATMTVDK | FDS | GTTL |
| | ID NO: 1241) | (SEQ ID | KSLEW | (SEQ ID | SSSTAYMEL | (SEQ ID | TVSS |
| | | NO: 19) | IGV | NO: 46) | ARLTSEDSAI | NO: 48) | (SEQ |
| | | | (SEQ ID | | YYC (SEQ ID | | ID |
| | | | NO: | | NO: 1389) | | NO: |
| | | | 1128) | | | | 425) |
| BP003-T2P1C8 | QIQLQQSGAELVRPG | GYTFT | LGWV | IYPGG | NYNEKFKG | VRVTP | WGQ |
| | TSVKISCKAS (SEQ | NYW | KQRPG | GYT | KATLTADTS | AS | GTTV |
| | ID NO: 1227) | (SEQ ID | HGLEW | (SEQ ID | PSTAYMQLS | (SEQ ID | TVSS |
| | | NO: 4) | IGD | NO: 6) | SLTSEDSAV | NO: | (SEQ |
| | | | (SEQ ID | | YFC (SEQ ID | 1443) | ID |
| | | | NO: | | NO: 1172) | | NO: |
| | | | 1075) | | | | 422) |
| BP003-T2P1F4 | QVQLQQPGAELVRP | GYTFT | IGWVK | IHPGG | NYNEKFKG | TSRNF | WGQ |
| | GTSVKMSCKAA | NYW | QRPGH | GYT | KATLTADTS | AY | GTLV |
| | (SEQ ID NO: 1315) | (SEQ ID | GLEWI | (SEQ ID | SSTAYMQLS | (SEQ ID | TVSA |
| | | NO: 4) | GD | NO: | SLTSEDSAIY | NO: | (SEQ |
| | | | (SEQ ID | 1015) | YC (SEQ ID | 1419) | ID |
| | | | NO: | | NO: 1177) | | NO: |
| | | | 1010) | | | | 421) |
| BP003-T2P1G2 | QVQLQQSGAELVRP | GYTFT | IGWVK | IHPGG | NYNEKFKG | TSRNF | WGQ |
| | GTSLKMSCKAA | NYW | QRPGH | GYT | KATLTADTS | AY | GTPV |
| | (SEQ ID NO: 1324) | (SEQ ID | GLEWI | (SEQ ID | SSTTYMQLS | (SEQ ID | TVSS |
| | | NO: 4) | GD | NO: | SLTSEDSAIY | NO: | (SEQ |
| | | | (SEQ ID | 1015) | YC (SEQ ID | 1419) | ID |
| | | | NO: | | NO: 1181) | | NO: |
| | | | 1010) | | | | 398) |
| BP003-T2P1G4 | QIQLQQSGAELVRPG | GYTFT | IGWVK | IHPGG | NYNEKFKG | TSRNF | WGQ |
| | TSLKMSCKAA (SEQ | NYW | QRPGH | GYT | KATLTADTS | AY | GTLV |
| | ID NO: 1226) | (SEQ ID | GLEWI | (SEQ ID | SSTTYMQLS | (SEQ ID | TVSA |
| | | NO: 4) | GD | NO: | SLTSEDSAIY | NO: | (SEQ |
| | | | (SEQ ID | 1015) | YC (SEQ ID | 1419) | ID |
| | | | NO: | | NO: 1181) | | NO: |
| | | | 1010) | | | | 421) |
| BP003-T2P1G7 | QIQLQQSGAELVRPG | GYTFT | IGWVK | INPGG | NYNEKFEGK | TSRNF | WGQ |
| | TSVKMSCKAA (SEQ | NYW | LRPGH | GYT | ATLTADTSS | AY | GTLV |
| | ID NO: 1228) | (SEQ ID | GLEWI | (SEQ ID | STAYMQLSS | (SEQ ID | TVSA |
| | | NO: 4) | GD | NO: | LTSEDSAIY | NO: | (SEQ |
| | | | (SEQ ID | 1023) | YC (SEQ ID | 1419) | ID |
| | | | NO: | | NO: 1168) | | NO: |
| | | | 1008) | | | | 421) |
| BP003-T2P1H5 | QIQLQQSGAELVRPG | GYTFT | IGWVK | IHPGG | NYNEKFKG | TSRNF | WGQ |
| | TSVKMSCKAA (SEQ | NYW | QRPGH | GYT | KATLTADTS | AY | GTLV |
| | ID NO: 1228) | (SEQ ID | GLEWV | (SEQ ID | SSTAYMQLS | (SEQ ID | TVSA |
| | | NO: 4) | GD | NO: | SLTSEDSAIY | NO: | (SEQ |
| | | | (SEQ ID | 1015) | YC (SEQ ID | 1419) | ID |
| | | | NO: | | NO: 1177) | | NO: |
| | | | 1011) | | | | 421) |
| BP003T3P2-1 | QVQLQQSGAELVRP | GYTFT | MHWV | ISTYSG | IYNQKFKGK | ARGVT | WGQ |
| | GVSLKISCKGS (SEQ | DYA | RQSHA | DA | ATMTVDKSS | FDY | GTTV |
| | ID NO: 1337) | (SEQ ID | KSLEW | (SEQ ID | STAYLELAR | (SEQ ID | TVSS |
| | | NO: 19) | IGV | NO: 21) | LTSDDSAIY | NO: 23) | (SEQ |
| | | | (SEQ ID | | YC (SEQ ID | | ID |
| | | | NO: | | NO: 1033) | | NO: |
| | | | 1128) | | | | 422) |
| BP003T3P2-10 | QVQLQQSGAELVRP | GYTFT | MHWV | ISTYSG | LYNQKFKG | ARGVT | WGQ |
| | GVSLKISCKGS (SEQ | DYA | RQSHA | DA | KATMTVDK | FDY | GTTL |
| | ID NO: 1337) | (SEQ ID | KSLEW | (SEQ ID | SSSTAYLEL | (SEQ ID | TVSS |
| | | NO: 19) | IGV | NO: 21) | ARLTSDDSA | NO: 23) | (SEQ |
| | | | (SEQ ID | | IYYC (SEQ | | ID |
| | | | NO: | | ID NO: | | NO: |
| | | | 1128) | | 1119) | | 425) |
| BP003T3P2-11 | QVQLQQSGAELVRP | GYTFT | MHWV | ISTYSG | LYNQKFKG | ARGVT | WGQ |
| | GVSLKISCKGS (SEQ | DYA | RQSHA | DA | KATMTVDK | FDY | GTTL |
| | ID NO: 1337) | (SEQ ID | KSLEW | (SEQ ID | SSSTAYLEL | (SEQ ID | TVSS |
| | | NO: 19) | IGV | NO: 21) | ARLTSDDSA | NO: 23) | (SEQ |
| | | | (SEQ ID | | IYYC (SEQ | | ID |
| | | | NO: | | ID NO: | | NO: |
| | | | 1128) | | 1119) | | 425) |
| BP003T3P2-12 | QAYLQQSGAELVRP | GYTFT | MHWV | ISTYSG | LYNQKFGK | ARGVT | WGQ |
| | GVSLKISCKGS (SEQ | DYA | RQSHA | DA | ATMTVDKSS | FDY | GTTL |
| | ID NO: 1195) | (SEQ ID | KSLEW | (SEQ ID | STAYLELAR | (SEQ ID | TVSS |
| | | NO: 19) | IGV | NO: 21) | LTSDDSAIY | NO: 23) | (SEQ |
| | | | (SEQ ID | | YC (SEQ ID | | ID |
| | | | NO: | | NO: 1118) | | NO: |
| | | | 1128) | | | | 425) |
| BP003T3P2-13 | QVQLQQSGAELVRP | GYTFT | MHWV | ISTYSG | IYNQKFGKA | ARGVT | WGQ |
| | GVSLKISCKGS (SEQ | DYA | RQSHA | DA | TMTVDKSSS | FDY | GTTV |
| | ID NO: 1337) | (SEQ ID | KSLEW | (SEQ ID | TAYLELARL | (SEQ ID | TVSS |
| | | NO: 19) | IGV | NO: 21) | TSDDSAIYY | NO: 23) | (SEQ |
| | | | (SEQ ID | | C (SEQ ID | | ID |
| | | | NO: | | NO: 1031) | | NO: |
| | | | 1128) | | | | 422) |
| BP003T3P2-14 | QIQLQQPGAELVRPG | GYTFT | MHWV | ISTYSG | IYNQKFGKA | ARGVT | WGQ |
| | VSLKISCKGS (SEQ | DYA | RQSHA | DA | TMTVDKSSS | FDY | GTTV |
| | ID NO: 1223) | (SEQ ID | KSLEW | (SEQ ID | TAYLELARL | (SEQ ID | TVSS |
| | | NO: 19) | IGV | NO: 21) | TSDDSAIYY | NO: 23) | (SEQ |
| | | | (SEQ ID | | C (SEQ ID | | ID |
| | | | NO: | | NO: 1031) | | NO: |
| | | | 1128) | | | | 422) |
| BP003T3P2-15 | QVQLQQSGAELVRP | GYTFT | MHWV | ISTYSG | IYNQKFGKA | ARGVT | WGQ |
| | GVSLKISCKGS (SEQ | DYA | RQSHA | DA | TMTVDKSSS | FDY | GTTV |
| | ID NO: 1337) | (SEQ ID | KSLEW | (SEQ ID | TAYLELARL | (SEQ ID | TVSS |
| | | NO: 19) | IGV | NO: 21) | TSDDSAIYY | NO: 23) | (SEQ |
| | | | (SEQ ID | | C (SEQ ID | | ID |
| | | | NO: | | NO: 1031) | | NO: |
| | | | 1128) | | | | 422) |
| BP003T3P2-16 | EVQLQQSGAELVRP | GYTFT | MHWV | ISTYSG | IYNQKFGKA | ARGVT | WGQ |
| | GVSLKISCKGS (SEQ | DYA | RQSHA | DA | TMTVDKSSS | FDY | GTTL |
| | ID NO: 940) | (SEQ ID | KSLEW | (SEQ ID | TAYLELARL | (SEQ ID | TVSS |
| | | NO: 19) | IGV | NO: 21) | TSDDSAIYY | NO: 23) | (SEQ |
| | | | (SEQ ID | | C (SEQ ID | | ID |
| | | | NO: | | NO: 1031) | | NO: |
| | | | 1128) | | | | 425) |
| BP003T3P2-17 | QIQLQQSGAELVRPG | GYTFT | MHWV | ISTYSG | IYNQKFGKA | ARGVT | WGQ |
| | VSLKISCKGS (SEQ | DYA | RQSHA | DA | TMTVDKSSS | FDY | GTSL |
| | ID NO: 1239) | (SEQ ID | KSLEW | (SEQ ID | TAYLELARL | (SEQ ID | TVSS |
| | | NO: 19) | IGV | NO: 21) | TSDDSAIYY | NO: 23) | (SEQ |
| | | | (SEQ ID | | C (SEQ ID | | ID |
| | | | NO: | | NO: 1031) | | NO: |
| | | | 1128) | | | | 424) |
| BP003T3P2-18 | QVQLQQSGAELVRP | GYTFT | MHWV | ISTYSG | IYNQKFGKA | ARGVT | WGQ |
| | GVSLKISCKGS (SEQ | DYA | RQSHA | DA | TMTVDKSSS | FDY | GTTL |
| | ID NO: 1337) | (SEQ ID | KSLEW | (SEQ ID | TAYLELARL | (SEQ ID | TVSS |
| | | NO: 19) | IGV | NO: 21) | TSDDSAIYY | NO: 23) | (SEQ |
| | | | (SEQ ID | | C (SEQ ID | | ID |
| | | | NO: | | NO: 1031) | | NO: |
| | | | 1128) | | | | 425) |
| BP003T3P2-19 | QVQLQQSGAELVRP | GYTFT | MHWV | ISTYSG | IYNQKFGKA | ARGVT | WGQ |
| | GVSLKISCKGS (SEQ | DYA | RQSHA | DA | TMTVDKSSS | FDY | GTTL |
| | ID NO: 1337) | (SEQ ID | KSLEW | (SEQ ID | TAYLELARL | (SEQ ID | TVSS |
| | | NO: 19) | IGV | NO: 21) | TSEDSAIYY | NO: 23) | (SEQ |
| | | | (SEQ ID | | C (SEQ ID | | ID |
| | | | NO: | | NO: 1032) | | NO: |
| | | | 1128) | | | | 425) |
| BP003T3P2-2 | QVQLQQSGAELVRP | GYTFT | MHWV | ISTYSG | IYNQKFKGK | ARGVT | WGQ |
| | GVSLKISCKGS (SEQ | DYA | RQSHA | DA | ATMTVDKSS | FDY | GTTV |
| | ID NO: 1337) | (SEQ ID | KSLEW | (SEQ ID | STAYLELAR | (SEQ ID | TVSS |
| | | NO: 19) | IGV | NO: 21) | LTSDDSAIY | NO: 23) | (SEQ |
| | | | (SEQ ID | | YC (SEQ ID | | ID |
| | | | NO: | | NO: 1033) | | NO: |
| | | | 1128) | | | | 422) |
| BP003T3P2-20 | QVQLQQSGAGLVRP | GYTFT | MHWV | ISTYSG | IYNQKFGKA | ARGVT | WGQ |
| | GVSLKISCKGS (SEQ | DYA | RQSHA | DA | TMTVDKSSS | FDY | GTTL |
| | ID NO: 1341) | (SEQ ID | KSLEW | (SEQ ID | TAYLELARL | (SEQ ID | TVSS |
| | | NO: 19) | IGV | NO: 21) | TSEDSAIYY | NO: 23) | (SEQ |
| | | | (SEQ ID | | C (SEQ ID | | ID |
| | | | NO: | | NO: 1032) | | NO: |
| | | | 1128) | | | | 425) |
| BP003T3P2-21 | QVQLQQSGAELVRP | GYTFT | MQWV | ISTYSG | IYNQKFGKA | ARGVT | WGQ |
| | GVSLKISCKGS (SEQ | DYA | RQSHA | DA | TMTVDKSSS | FDY | GTSL |
| | ID NO: 1337) | (SEQ ID | KSLEW | (SEQ ID | TAYLELARL | (SEQ ID | TVSS |
| | | NO: 19) | IGV | NO: 21) | TSEDSAIYY | NO: 23) | (SEQ |
| | | | (SEQ ID | | C (SEQ ID | | ID |
| | | | NO: | | NO: 1032) | | NO: |
| | | | 1137) | | | | 424) |
| BP003T3P2-22 | QVQLQQSGAELVRP | GYTFT | MHWV | ISTYSG | IYNQKFDKA | ARGVT | WGQ |
| | GVSLKISCKGS (SEQ | DYA | RQSHA | DA | TMTVDKSSS | FDY | GTTL |
| | ID NO: 1337) | (SEQ ID | KSLEW | (SEQ ID | TAYLELARL | (SEQ ID | TVSS |
| | | NO: 19) | IGV | NO: 21) | TSEDSAIYY | NO: 23) | (SEQ |
| | | | (SEQ ID | | C (SEQ ID | | ID |
| | | | NO: | | NO: 1030) | | NO: |
| | | | 1128) | | | | 425) |
| BP003T3P2-23 | QVQLQQSGAELVRP | GYTFT | MHWV | ISTYSG | SYNQKFKG | ARGVT | WGQ |
| | GVSVKISCKGS (SEQ | DYA | KQSHA | DV | KATMTVDK | FDS | GTTL |
| | ID NO: 1338) | (SEQ ID | KSLEW | (SEQ ID | SSSTAYMEL | (SEQ ID | TVSS |
| | | NO: 19) | IGV | NO: 46) | ARLTSEDSAI | NO: 48) | (SEQ |
| | | | (SEQ ID | | YYC (SEQ ID | | ID |
| | | | NO: | | NO: 1389) | | NO: |
| | | | 1126) | | | | 425) |
| BP003T3P2-24 | QVQLQQSGAELVRP | GYTFT | MHWV | ISTYSG | SYNQKFKG | ARGVT | WGQ |
| | GVSVKISCKGS (SEQ | DYA | KQSHA | DV | KATMTVDK | FDS | GTTL |
| | ID NO: 1338) | (SEQ ID | KSLEW | (SEQ ID | SSSTAYMEL | (SEQ ID | TVSS |
| | | NO: 19) | IGV | NO: 46) | ARLTSEDSAI | NO: 48) | (SEQ |
| | | | (SEQ ID | | YYC (SEQ ID | | ID |
| | | | NO: | | NO: 1389) | | NO: |
| | | | 1126) | | | | 425) |
| BP003T3P2-25 | QIQLQQSGAELVRPG | GYTFT | MHWV | ISTYSG | SYNQKFKG | ARGVT | WGQ |
| | VSVKISCKGS (SEQ | DYA | KQSHA | DV | KATMTVDK | FDS | GTSL |
| | ID NO: 1240) | (SEQ ID | KSLEW | (SEQ ID | SSSTAYMEL | (SEQ ID | TVSS |
| | | NO: 19) | IGV | NO: 46) | ARLTSEDSAI | NO: 48) | (SEQ |
| | | | (SEQ ID | | YYC (SEQ ID | | ID |
| | | | NO: | | NO: 1389) | | NO: |
| | | | 1126) | | | | 424) |
| BP003T3P2-26 | QVQLQQSGAELVRP | GYTFT | MRWV | IXTYSG | SYNQKFGK | ARGVT | WGR |
| | GISVKISCKGS (SEQ | DYA | KQSHA | DV | ATMTVDKSS | FDS | GTSL |
| | ID NO: 1323) | (SEQ ID | KSLEW | (SEQ ID | STAYMELAR | (SEQ ID | TVSS |
| | | NO: 19) | IGV | NO: | LTSEDSAIY | NO: 48) | (SEQ |
| | | | (SEQ ID | 1029) | YC (SEQ ID | | ID |
| | | | NO: | | NO: 1387) | | NO: |
| | | | 1138) | | | | 1454) |
| BP003T3P2-27 | QVQLQQSGAELVRP | GYTFT | MRWV | ISTYSG | NYNQKFGK | ARGVT | XGR |
| | GISVKISCKGS (SEQ | DYG | KQSPA | DV | ATMTVDKSS | FDS | GTXL |
| | ID NO: 1323) | (SEQ ID | XSLEW | (SEQ ID | STAYMELAR | (SEQ ID | TVSS |
| | | NO: | IGV | NO: 46) | LTSEDSAIY | NO: 48) | (SEQ |
| | | 987) | (SEQ ID | | YC (SEQ ID | | ID |
| | | | NO: | | NO: 1190) | | NO: |
| | | | 1139) | | | | 1474) |
| BP003T3P2-28 | QVQLQQSGAELAKP | GYTFT | IHWVK | INPRTD | EYNQKFKD | ARHGY | WGQ |
| | GASVKMSCKAS | RYW | QRPGQ | YT | KATLTADKS | FDY | GTTL |
| | (SEQ ID NO: 473) | (SEQ ID | DLEWI | (SEQ ID | SSTAYMQLS | (SEQ ID | TVSS |
| | | NO: | GY | NO: | SLTSDDSAV | NO: | (SEQ |
| | | 992) | (SEQ ID | 1025) | YYC | 611) | ID |
| | | | NO: | | (SEQ ID | | NO: |
| | | | 1018) | | NO: 955) | | 425) |
| BP003T3P2-29 | QVQLQQPGAELAKP | GYTFT | IHWVK | INPRTD | EYNQKFKD | ARHGY | WGQ |
| | GASVKMSCKAS | RYW | QRPGQ | YT | KATLTADKS | FDY | GTTL |
| | (SEQ ID NO: 114) | (SEQ ID | DLEWI | (SEQ ID | SSTAYMQLS | (SEQ ID | TVSS |
| | | NO: | GY | NO: | SLTSDDSAV | NO: | (SEQ |
| | | 992) | (SEQ ID | 1025) | YYC (SEQ ID | 611) | ID |
| | | | NO: | | NO: 955) | | NO: |
| | | | 1018) | | | | 425) |
| BP003T3P2-3 | QVQLQQSGAELVRP | GYTFT | MHWV | ISTYSG | IYNQKFKGK | ARGVT | WGQ |
| | GVSLKISCKGS (SEQ | DYA | RQSHA | DA | ATMTVDKSS | FDY | GTTV |
| | ID NO: 1337) | (SEQ ID | KSLEW | (SEQ ID | STAYLELAR | (SEQ ID | TVSS |
| | | NO: 19) | IGV | NO: 21) | LTSDDSAIY | NO: 23) | (SEQ |
| | | | (SEQ ID | | YC (SEQ ID | | ID |
| | | | NO: | | NO: 1033) | | NO: |
| | | | 1128) | | | | 422) |
| BP003T3P2-30 | QVQLQQPGAELAKP | GYTFT | IHWVK | INPRTD | EYNQKFKD | ARHGY | WGQ |
| | GASVKMSCKAS | RYW | QRPGQ | YT | KATLTADKS | FDY | GTTL |
| | (SEQ ID NO: 114) | (SEQ ID | DLEWI | (SEQ ID | SSTAYMQLS | (SEQ ID | TVSS |
| | | NO: | GY | NO: | SLTSDDSAV | NO: | (SEQ |
| | | 992) | (SEQ ID | 1025) | YYC | 611) | ID |
| | | | NO: | | (SEQ ID | | NO: |
| | | | 1018) | | NO: 955) | | 425) |
| BP003T3P2-31 | QVQLQQPGAELAKP | GYTFT | IHWVK | INPRTD | EYNQKFKD | ARHGY | WGQ |
| | GASVKMSCKAS | RYW | QRPGQ | YT | KATLTADKS | FDY | GTTL |
| | (SEQ ID NO: 114) | (SEQ ID | DLEWI | (SEQ ID | SSTAYMQLS | (SEQ ID | TVSS |
| | | NO: | GY | NO: | SLTSDDSAV | NO: | (SEQ |
| | | 992) | (SEQ ID | 1025) | YYC | 611) | ID |
| | | | NO: | | (SEQ ID | | NO: |
| | | | 1018) | | NO: 955) | | 425) |
| BP003T3P2-32 | QVQLQQSGAELAKP | GYTFT | IHWVK | INPRTD | EYNQKFDK | ARHGY | WGQ |
| | GASVKMSCKAS | RYW | QRPGQ | YT | ATLTADKSS | FDY | GTTL |
| | (SEQ ID NO: 473) | (SEQ ID | DLEWI | (SEQ ID | STAYMQLXS | (SEQ ID | TVSS |
| | | NO: | GY | NO: | LTSDDSAVY | NO: | (SEQ |
| | | 992) | (SEQ ID | 1025) | YC (SEQ ID | 611) | ID |
| | | | NO: | | NO: 952) | | NO: |
| | | | 1018) | | | | 425) |
| BP003T3P2-33 | QVQLQQPGAELAKP | GYTFT | IHWVK | INPRTD | EYNQKFDK | ARHGY | WGQ |
| | GASVKMSCKAS | RYW | QRPGQ | YT | ATLTADKSS | FDY | GTTL |
| | (SEQ ID NO: 114) | (SEQ ID | DLEWI | (SEQ ID | STAYMQLSS | (SEQ ID | TVSS |
| | | NO: | GY | NO: | LTSDDSAVY | NO: | (SEQ |
| | | 992) | (SEQ ID | 1025) | YC (SEQ ID | 611) | ID |
| | | | NO: | | NO: 951) | | NO: |
| | | | 1018) | | | | 425) |
| BP003T3P2-34 | QVQLQQPGAELAKP | GYTFT | IHWVK | INPRTD | EYNQKFDK | ARHGY | WGQ |
| | GASVKMSCKAS | RYW | QRPGQ | YT | ATLTADKSS | FDY | GTTL |
| | (SEQ ID NO: 114) | (SEQ ID | DLEWI | (SEQ ID | STAYMQLSS | (SEQ ID | TVSS |
| | | NO: | GY | NO: | LTSDDSAVY | NO: | (SEQ |
| | | 992) | (SEQ ID | 1025) | YC (SEQ ID | 611) | ID |
| | | | NO: | | NO: 951) | | NO: |
| | | | 1018) | | | | 425) |
| BP003T3P2-35 | QVQLQQSGAELAKP | GYTFT | IHWVK | INPRTD | EYNQKFDK | ARHGY | WGQ |
| | GASVKMSCKAS | RYW | QRPGQ | YT | ATLTADKSS | FDY | GTTL |
| | (SEQ ID NO: 473) | (SEQ ID | DLEWI | (SEQ ID | STAYMQLSS | (SEQ ID | TVSS |
| | | NO: | GY | NO: | LTSDDSAVY | NO: | (SEQ |
| | | 992) | (SEQ ID | 1025) | YC (SEQ ID | 611) | ID |
| | | | NO: | | NO: 951) | | NO: |
| | | | 1018) | | | | 425) |
| BP003T3P2-36 | QVQLQQSGAELAKP | GYTFT | MHWV | INPRTD | EYNQKFDK | ARHGY | WGQ |
| | GASVKMSCKAS | RYW | KQRPG | YT | ATLTADKSS | FDY | GTTL |
| | (SEQ ID NO: 473) | (SEQ ID | QDLEW | (SEQ ID | STAYMQLSS | (SEQ ID | TVSS |
| | | NO: | IGY | NO: | LTSDDSAVY | NO: | (SEQ |
| | | 992) | (SEQ ID | 1025) | YC (SEQ ID | 611) | ID |
| | | | NO: | | NO: 951) | | NO: |
| | | | 1125) | | | | 425) |
| BP003T3P2-37 | QIQLQQPGAELAKPG | GYTFT | MHWV | INPRTD | EYNQKFDK | ARHGY | WGQ |
| | ASVKMSCKAS (SEQ | RYW | KQRPG | YT | ATLTADKSS | FDY | GTTV |
| | ID NO: 1218) | (SEQ ID | QDLEW | (SEQ ID | STAYMQLSS | (SEQ ID | TVSS |
| | | NO: | IGY | NO: | LTSDDSAVY | NO: | (SEQ |
| | | 992) | (SEQ ID | 1025) | YC (SEQ ID | 611) | ID |
| | | | NO: | | NO: 951) | | NO: |
| | | | 1125) | | | | 422) |
| BP003T3P2-38 | QVQMKQSGAELAKP | GYTFT | MHWV | INPSSD | EYNQKFKD | ARGTV | WGQ |
| | GASVKMSCKAS | RYW | KQRPG | YT | KATLTADKS | WDY | GTTV |
| | (SEQ ID NO: 1343) | (SEQ ID | QGLEW | (SEQ ID | SSTAYMQLS | (SEQ ID | TVSS |
| | | NO: | IGY | NO: | SLTSEDSAV | NO: | (SEQ |
| | | 992) | (SEQ ID | 1026) | YYC (SEQ ID | 608) | ID |
| | | | NO: | | NO: 265) | | NO: |
| | | | 190) | | | | 422) |
| BP003T3P2-39 | QIQLQQSGAELAKPG | GYTFT | MHWV | INPSTD | EYNEKFDKA | VRSPIL | WGQ |
| | ASVKMSCKAS (SEQ | RYW | KQRPG | YT | TLTADKSSS | DY | GTSL |
| | ID NO: 1225) | (SEQ ID | QGLEW | (SEQ ID | TAYMQLSSL | (SEQ ID | TVSS |
| | | NO: | IGY | NO: | TSEDSAVYF | NO: | (SEQ |
| | | 992) | (SEQ ID | 1027) | C (SEQ ID | 1442) | ID |
| | | | NO: | | NO: 949) | | NO: |
| | | | 190) | | | | 424) |
| BP003T3P2-4 | QVQLQQSGAELVRP | GYTFT | MHWV | ISTYSG | IYNQKFKGK | ARGVT | WGQ |
| | GVSLKISCKGS (SEQ | DYA | RQSHA | DA | ATMTVDKSS | FDY | GTTV |
| | ID NO: 1337) | (SEQ ID | KSLEW | (SEQ ID | STAYLELAR | (SEQ ID | TVSS |
| | | NO: 19) | IGV | NO: 21) | LTSDDSAIY | NO: 23) | (SEQ |
| | | | (SEQ ID | | YC (SEQ ID | | ID |
| | | | NO: | | NO: 1033) | | NO: |
| | | | 1128) | | | | 422) |
| BP003T3P2-40 | QVQLQQSGAELVRP | GYTFT | LGWV | IYPGG | NYNEKFKG | ARVTP | WGQ |
| | GTSVKISCKAS (SEQ | NYW | KQRPG | GYT | KATLTADTS | AS | GTSL |
| | ID NO: 1325) | (SEQ ID | HGLEW | (SEQ ID | SSTAYMQLS | (SEQ ID | TVSS |
| | | NO: 4) | IGD | NO: 6) | SLTSEDSAV | NO: 8) | (SEQ |
| | | | (SEQ ID | | YFC (SEQ ID | | ID |
| | | | NO: | | NO: 1178) | | NO: |
| | | | 1075) | | | | 424) |
| BP003T3P2-41 | QAYLQQSGAELVRP | GYTFT | LGWV | IYPGG | NYNEKFKG | ARVTP | WGQ |
| | GTSVKISCKAS (SEQ | NYW | KQRPG | GYT | KATLTADTS | AS | GTSL |
| | ID NO: 1193) | (SEQ ID | HGLEW | (SEQ ID | SSTAYMQLS | (SEQ ID | TVSS |
| | | NO: 4) | IGD | NO: 6) | SLTSEDSAV | NO: 8) | (SEQ |
| | | | (SEQ ID | | YFC (SEQ ID | | ID |
| | | | NO: | | NO: 1178) | | NO: |
| | | | 1075) | | | | 424) |
| BP003T3P2-42 | QVQLQQPGAELVRP | GYTFT | LGWV | IYPGG | NYNEKFKG | ARVTP | WGQ |
| | GTSVKISCKAS (SEQ | NYW | KQRPG | GYT | KATLTADTS | AS | GTTL |
| | ID NO: 1314) | (SEQ ID | HGLEW | (SEQ ID | SSTAYMQLS | (SEQ ID | TVSS |
| | | NO: 4) | IGD | NO: 6) | SLTSEDSAV | NO: 8) | (SEQ |
| | | | (SEQ ID | | YFC (SEQ ID | | ID |
| | | | NO: | | NO: 1178) | | NO: |
| | | | 1075) | | | | 425) |
| BP003T3P2-43 | QVQLQQSGAELVRP | GYTFT | LGWV | IYPGG | NYNEKFKG | ARVTP | WGQ |
| | GTSVKISCKAS (SEQ | NYW | KQRPG | GYT | KATLTADTS | AS | GTTV |
| | ID NO: 1325) | (SEQ ID | HGLEW | (SEQ ID | SSTAYMQLS | (SEQ ID | TVSS |
| | | NO: 4) | IGD | NO: 6) | SLTSEDSAV | NO: 8) | (SEQ |
| | | | (SEQ ID | | YFC (SEQ ID | | ID |
| | | | NO: | | NO: 1178) | | NO: |
| | | | 1075) | | | | 422) |
| BP003T3P2-44 | QVQLQPGAELVRPG | GYTFT | LGWV | IYPGG | NYNEKFKG | ARVTP | WGQ |
| | TSVKISCKAS (SEQ | NYW | KQRPG | GYT | KATLTADTS | AS | GTTV |
| | ID NO: 1313) | (SEQ ID | HGLEW | (SEQ ID | SSTAYMQLS | (SEQ ID | TVSS |
| | | NO: 4) | IGD | NO: 6) | SLTSEDSAV | NO: 8) | (SEQ |
| | | | (SEQ ID | | YFC (SEQ ID | | ID |
| | | | NO: | | NO: 1178) | | NO: |
| | | | 1075) | | | | 422) |
| BP003T3P2-45 | QVQLQQSGAELVRP | GYTFT | LGWV | IYPGG | NYNEKFKG | ARVTP | WGQ |
| | GTSVKISCKAS (SEQ | NYW | KQRPG | GYT | KATLTADTS | AS | GTTL |
| | ID NO: 1325) | (SEQ ID | HGLEW | (SEQ ID | SSTAYMQLS | (SEQ ID | TVSS |
| | | NO: 4) | IGD | NO: 6) | SLTSEDSAV | NO: 8) | (SEQ |
| | | | (SEQ ID | | YFC (SEQ ID | | ID |
| | | | NO: | | NO: 1178) | | NO: |
| | | | 1075) | | | | 425) |
| BP003T3P2-46 | QVQLQQSGAELVRP | GYTFT | LGWV | IYPGG | NYNEKFGK | ARVTP | WGQ |
| | GTSVKISCKAS (SEQ | NYW | KQRPG | GYT | ATLTADTSS | AS | GTTV |
| | ID NO: 1325) | (SEQ ID | HGLEW | (SEQ ID | STAYMQLSS | (SEQ ID | TVSS |
| | | NO: 4) | IGD | NO: 6) | LTSEDSAVY | NO: 8) | (SEQ |
| | | | (SEQ ID | | FC (SEQ ID | | ID |
| | | | NO: | | NO: 1170) | | NO: |
| | | | 1075) | | | | 422) |
| BP003T3P2-47 | QVQLQQSGAELVRP | GYTFT | LGWV | IYPGG | NYNEKFGK | ARVTP | WGQ |
| | GASVKISCKAS (SEQ | NYW | KQRPG | GYT | ATLTADTSS | AS | GTTL |
| | ID NO: 1322) | (SEQ ID | HGLEW | (SEQ ID | STAYMQLSS | (SEQ ID | TVSS |
| | | NO: 4) | IGD | NO: 6) | LTSEDSAVY | NO: 8) | (SEQ |
| | | | (SEQ ID | | FC (SEQ ID | | ID |
| | | | NO: | | NO: 1170) | | NO: |
| | | | 1075) | | | | 425) |
| BP003T3P2-48 | QVQLQQSGAELVRP | GYTFT | LGWIK | IYPGG | NYNEKFGK | ARVTP | WGQ |
| | GTSVKISCKAS (SEQ | NYW | QRPGH | GYT | ATLTADTSS | AS | GTSL |
| | ID NO: 1325) | (SEQ ID | GLEWI | (SEQ ID | STAYMQLSS | (SEQ ID | TVSS |
| | | NO: 4) | GD | NO: 6) | LTSEDSAVY | NO: 8) | (SEQ |
| | | | (SEQ ID | | FC (SEQ ID | | ID |
| | | | NO: | | NO: 1170) | | NO: |
| | | | 1074) | | | | 424) |
| BP003T3P2-49 | QVQLQQSGAELVRP | GYTFT | LGWV | IYPGG | NYNEKFGK | ARVTP | WGQ |
| | GTSVKISCKAS (SEQ | NYW | KQRPG | GYT | ATLTADTSS | AS | GTTV |
| | ID NO: 1325) | (SEQ ID | HGLEW | (SEQ ID | STAYMQLSS | (SEQ ID | TVSS |
| | | NO: 4) | IGD | NO: 6) | LTSEDSAVY | NO: 8) | (SEQ |
| | | | (SEQ ID | | FC (SEQ ID | | ID |
| | | | NO: | | NO: 1170) | | NO: |
| | | | 1075) | | | | 422) |
| BP003T3P2-5 | QIQLQQSGAELVRPG | GYTFT | MHWV | ISTYSG | IYNQKFKGK | ARGVT | WGQ |
| | VSLKISCKGS (SEQ | DYA | RQSHA | DA | ATMTVDKSS | FDY | GTSL |
| | ID NO: 1239) | (SEQ ID | KSLEW | (SEQ ID | STAYLELAR | (SEQ ID | TVSS |
| | | NO: 19) | IGV | NO: 21) | LTSDDSAIY | NO: 23) | (SEQ |
| | | | (SEQ ID | | YC (SEQ ID | | ID |
| | | | NO: | | NO: 1033) | | NO: |
| | | | 1128) | | | | 424) |
| BP003T3P2-50 | QVQLQQSGAELVRP | GYTFT | LGWV | IYPGG | NYNEKFGK | ARVTP | WGQ |
| | GTSVKISCKAS (SEQ | NYW | KQRPG | GYT | ATLTADTSS | AS | GTTL |
| | ID NO: 1325) | (SEQ ID | HGLEW | (SEQ ID | STAYMQLSS | (SEQ ID | TVSS |
| | | NO: 4) | IGD | NO: 6) | LTSEDSAVY | NO: 8) | (SEQ |
| | | | (SEQ ID | | FC (SEQ ID | | ID |
| | | | NO: | | NO: 1170) | | NO: |
| | | | 1075) | | | | 425) |
| BP003T3P2-51 | QVQLQQSGAELVRP | GYTFT | LGWV | IYPGG | NYNEKFGK | ARVTP | WGQ |
| | GTSVKISCKAS (SEQ | NYW | KQRPG | GYT | ATLTADTSS | AS | GTTV |
| | ID NO: 1325) | (SEQ ID | HGLEW | (SEQ ID | STAYMQLSS | (SEQ ID | TVSS |
| | | NO: 4) | IGD | NO: 6) | LTSEDSAVY | NO: 8) | (SEQ |
| | | | (SEQ ID | | FC (SEQ ID | | ID |
| | | | NO: | | NO: 1170) | | NO: |
| | | | 1075) | | | | 422) |
| BP003T3P2-52 | QVQLQQSGAELVRP | GYTFT | LGWV | IYPGG | NYNEKFGK | ARVTP | WGQ |
| | GTSVKISCKAS (SEQ | NYW | KQRPG | GYT | ATLTADTSS | AS | GTTV |
| | ID NO: 1325) | (SEQ ID | HGLEW | (SEQ ID | STAYMQLSS | (SEQ ID | TVSS |
| | | NO: 4) | IGD | NO: 6) | LTSEDSAVY | NO: 8) | (SEQ |
| | | | (SEQ ID | | FC (SEQ ID | | ID |
| | | | NO: | | NO: 1170) | | NO: |
| | | | 1075) | | | | 422) |
| BP003T3P2-53 | QVQLQQSGAELVRP | GYTFT | LGWV | IYPGG | NYNEKFGK | ARVTP | WGQ |
| | GTSVKISCKAS (SEQ | NYW | KQRPG | GYT | ATLTADTSS | AS | GTTL |
| | ID NO: 1325) | (SEQ ID | HGLEW | (SEQ ID | STAYMQLSS | (SEQ ID | TVSS |
| | | NO: 4) | IGD | NO: 6) | LTSEDSAVY | NO: 8) | (SEQ |
| | | | (SEQ ID | | FC (SEQ ID | | ID |
| | | | NO: | | NO: 1170) | | NO: |
| | | | 1075) | | | | 425) |
| BP003T3P2-54 | QVQLQQSGAELVRP | GYTFT | LGWV | IYPGG | NYNEKFGK | ARVTP | WGQ |
| | GTSVKISCKAS (SEQ | NYW | KQRPG | GYA | ATLTADTSS | AS | GTTL |
| | ID NO: 1325) | (SEQ ID | HGLEW | (SEQ ID | STAYMQLSS | (SEQ ID | TVSS |
| | | NO: 4) | IGD | NO: | LTSEDSAVY | NO: 8) | (SEQ |
| | | | (SEQ ID | 1035) | FC (SEQ ID | | ID |
| | | | NO: | | NO: 1170) | | NO: |
| | | | 1075) | | | | 425) |
| BP003T3P2-55 | QIQLQQSGAELVRPG | GYTFT | LGWV | IYPGG | NYNEKFGK | ARVTP | WGQ |
| | TSVKISCKAS (SEQ | NYW | KQRPG | GYT | ATLTADTSS | AS | GTTL |
| | ID NO: 1227) | (SEQ ID | HGLEW | (SEQ ID | STAYMQLSS | (SEQ ID | TVSS |
| | | NO: 4) | IGD | NO: 6) | LTSEDSAVY | NO: 8) | (SEQ |
| | | | (SEQ ID | | FC (SEQ ID | | ID |
| | | | NO: | | NO: 1170) | | NO: |
| | | | 1075) | | | | 425) |
| BP003T3P2-56 | QIQLQQSGAELVRPG | GYTFT | LGWV | IYPGG | NYNEKFGK | ARVTP | WGQ |
| | TSVKISCKAS (SEQ | NYW | KQRPG | GYT | ATLTADTSS | AS | GTSL |
| | ID NO: 1227) | (SEQ ID | HGLEW | (SEQ ID | STAYMQLSS | (SEQ ID | TVSS |
| | | NO: 4) | IGD | NO: 6) | LTSEDSAVY | NO: 8) | (SEQ |
| | | | (SEQ ID | | FC (SEQ ID | | ID |
| | | | NO: | | NO: 1170) | | NO: |
| | | | 1075) | | | | 424) |
| BP003T3P2-57 | QIQLQQSGAELVRPG | GYTFT | LGWV | IYPGG | NYNEKFGK | ARVTP | WGQ |
| | TSVKISCKAS (SEQ | NYW | KQRPG | GYT | ATLTADTSS | AS | GTTL |
| | ID NO: 1227) | (SEQ ID | HGLEW | (SEQ ID | STAYMQLSS | (SEQ ID | TVSS |
| | | NO: 4) | IGD | NO: 6) | LTSEDSAVY | NO: 8) | (SEQ |
| | | | (SEQ ID | | FC (SEQ ID | | ID |
| | | | NO: | | NO: 1170) | | NO: |
| | | | 1075) | | | | 425) |
| BP003T3P2-58 | QIQLQQSGAELVRPG | GYTFT | LGWV | IYPGG | NYNEKFGK | ARVTP | WGQ |
| | TSVKISCKAS (SEQ | NYW | KQRPG | GYT | ATLTADTSS | AS | GTSL |
| | ID NO: 1227) | (SEQ ID | HGLEW | (SEQ ID | STAYMQLSS | (SEQ ID | TVSS |
| | | NO: 4) | IGD | NO: 6) | LTSEDSAVY | NO: 8) | (SEQ |
| | | | (SEQ ID | | FC (SEQ ID | | ID |
| | | | NO: | | NO: 1170) | | NO: |
| | | | 1075) | | | | 424) |
| BP003T3P2-59 | QIQLQQSGAELVRPG | GYTFT | LGWV | IYPGG | NYNEKFKK | ARVTP | WGQ |
| | TSVKISCKAS (SEQ | NYW | KQRPG | GYT | ATLTADTSS | AS | GTTV |
| | ID NO: 1227) | (SEQ ID | HGLEW | (SEQ ID | VTAYMQLSS | (SEQ ID | TVSS |
| | | NO: 4) | IGD | NO: 6) | LTSEDSAVY | NO: 8) | (SEQ |
| | | | (SEQ ID | | FC (SEQ ID | | ID |
| | | | NO: | | NO: 1183) | | NO: |
| | | | 1075) | | | | 422) |
| BP003T3P2-6 | EVQLQQSGAELVRP | GYTFT | MHWV | ISTYSG | IYNQKFKGK | ARGVT | WGQ |
| | GVSLKISCKGS (SEQ | DYA | RQSHA | DA | ATMTVDKSS | FDY | GTTL |
| | ID NO: 940) | (SEQ ID | KSLEW | (SEQ ID | STAYLELAR | (SEQ ID | TVSS |
| | | NO: 19) | IGV | NO: 21) | LTSDDSAIY | NO: 23) | (SEQ |
| | | | (SEQ ID | | YC (SEQ ID | | ID |
| | | | NO: | | NO: 1033) | | NO: |
| | | | 1128) | | | | 425) |
| BP003T3P2-60 | QVQLQQSGAELVRP | GYTFT | LGWV | IYPGG | NYNEKFKK | ARVTP | WGQ |
| | GTSVKISCKAS (SEQ | NYW | KQRPG | GYT | ATLTADTSS | AS | GTTL |
| | ID NO: 1325) | (SEQ ID | HGLEW | (SEQ ID | STAYMQLSS | (SEQ ID | TVSS |
| | | NO: 4) | IGD | NO: 6) | LTSEDSAVY | NO: 8) | (SEQ |
| | | | (SEQ ID | | FC (SEQ ID | | ID |
| | | | NO: | | NO: 1182) | | NO: |
| | | | 1075) | | | | 425) |
| BP003T3P2-61 | QVQLQQSGAELVRP | GYTFT | LGWV | IYPGG | NYNEKFKK | ARVTP | WGQ |
| | GTSVKISCKAS (SEQ | NYW | KQRPG | GYT | ATLTADTSS | AS | GTSL |
| | ID NO: 1325) | (SEQ ID | HGLEW | (SEQ ID | STAYMQLSS | (SEQ ID | TVSS |
| | | NO: 4) | IGD | NO: 6) | LTSEDSAVY | NO: 8) | (SEQ |
| | | | (SEQ ID | | FC (SEQ ID | | ID |
| | | | NO: | | NO: 1182) | | NO: |
| | | | 1075) | | | | 424) |
| BP003T3P2-62 | EVQLQESGADLMKP | GYTFS | IEWIKQ | ILPGSG | NYNENFGK | ARGGT | WGQ |
| | GASVKISCKAS (SEQ | NYW | RPGHG | FT (SEQ | ATFTADTSS | SVVHF | GTTL |
| | ID NO: 939) | (SEQ ID | LEWVG | ID NO: | NTTYMQLSS | DY | TVSS |
| | | NO: | E (SEQ | 1021) | LTSEDSAVY | (SEQ ID | (SEQ |
| | | 986) | ID NO: | | YC (SEQ ID | NO: | ID |
| | | | 1006) | | NO: 1185) | 605) | NO: |
| | | | | | | | 425) |
| BP003T3P2-63 | QVQLQQSGADLMKP | GYTFS | IEWIKQ | ILPGSG | NYNENFKG | ARGGT | WGQ |
| | GASVKMSCRAS | NYW | RPGHG | FT (SEQ | KATFTADTS | SVVHF | GTSL |
| | (SEQ ID NO: 1321) | (SEQ ID | LEWIG | ID NO: | SNTTYMLLS | DS | TVSS |
| | | NO: | E (SEQ | 1021) | SLSSEDSAV | (SEQ ID | (SEQ |
| | | 986) | ID NO: | | YYC (SEQ ID | NO: | ID |
| | | | 1005) | | NO: 1187) | 604) | NO: |
| | | | | | | | 424) |
| BP003T3P2-64 | QIQLQQSGADLMKP | GYTFS | IEWIKQ | ILPGSG | NYNENFKG | ARGGT | WGQ |
| | GASVKISCKAS (SEQ | NYW | RPGHG | FT (SEQ | KATFTADTS | SVVHF | GTTL |
| | ID NO: 1224) | (SEQ ID | LEWVG | ID NO: | SNTTYMQLS | DY | TVSS |
| | | NO: | E (SEQ | 1021) | SLTSEDSAV | (SEQ ID | (SEQ |
| | | 986) | ID NO: | | YYC (SEQ ID | NO: | ID |
| | | | 1006) | | NO: 1188) | 605) | NO: |
| | | | | | | | 425) |
| BP003T3P2-65 | EVLLQQSGADLMKP | GYTFS | IEWIKQ | ILPGSG | NYNENFKG | ARGGT | WGQ |
| | GASVKISCKAS (SEQ | NYW | RPGHG | FT (SEQ | KATFTADTS | SVVHF | GTTV |
| | ID NO: 938) | (SEQ ID | LEWIG | ID NO: | SNTTYMQLS | DFDY | TVSS |
| | | NO: | E (SEQ | 1021) | SLTSEDSAV | (SEQ ID | (SEQ |
| | | 986) | ID NO: | | YYC (SEQ ID | NO: | ID |
| | | | 1005) | | NO: 1188) | 603) | NO: |
| | | | | | | | 422) |
| BP003T3P2-66 | EVRLQQSGAELVRS | GFNIK | MHWV | IDPENG | EYAPKFQGK | NVITT | WGQ |
| | GASVKLSCTAS (SEQ | DYY | KQRPE | DT | ATMTADTSS | ATTWF | GTLV |
| | ID NO: 943) | (SEQ ID | QGLEW | (SEQ ID | NTAYLQLSS | AY | TVSS |
| | | NO: | IGW | NO: | LTSEDTAVY | (SEQ ID | (SEQ |
| | | 977) | (SEQ ID | 1004) | YC (SEQ ID | NO: | ID |
| | | | NO: | | NO: 947) | 1165) | NO: |
| | | | 1124) | | | | 413) |
| BP003T3P2-67 | EVLLQQFGAELVRSG | GFNIK | IHWVK | IDPDN | EYAPKFQGK | TVFWY | WGQ |
| | ASVKLSCTAS (SEQ | DYY | QRPEQ | GET | ATMTTDTSS | GNNYA | GTLV |
| | ID NO: 937) | (SEQ ID | GLEWI | (SEQ ID | NTAHLQLSS | GFAY | TVSS |
| | | NO: | GW | NO: | LTSEDTAVY | (SEQ ID | (SEQ |
| | | 977) | (SEQ ID | 1003) | YC (SEQ ID | NO: | ID |
| | | | NO: | | NO: 948) | 1420) | NO: |
| | | | 1017) | | | | 413) |
| BP003T3P2-68 | EVHLQQSGAELVRS | GFNIK | IHWVK | IDPDN | EYAPKFQGK | TVFWY | WGA |
| | GASVKLSCTAS (SEQ | DYY | QRPEQ | GET | ATMTADTSS | GNNYA | GTSL |
| | ID NO: 936) | (SEQ ID | GLEWI | (SEQ ID | NTAHLQLSS | GFAY | TVSS |
| | | NO: | GW | NO: | LTSEDTAVY | (SEQ ID | (SEQ |
| | | 977) | (SEQ ID | 1003) | YC (SEQ ID | NO: | ID |
| | | | NO: | | NO: 946) | 1420) | NO: |
| | | | 1017) | | | | 431) |
| BP003T3P2-69 | EVRLQQSGAELVRS | GFNIK | IHWVK | IDPDN | EYAPKFQGK | TVFWY | WGQ |
| | GASVKLSCTAS (SEQ | DYY | QRPEQ | GET | ATMTADTSS | GNNYA | GTLV |
| | ID NO: 943) | (SEQ ID | GLEWI | (SEQ ID | NTAHLQLSS | GFAY | TVSA |
| | | NO: | GW | NO: | LTSEDTAVY | (SEQ ID | (SEQ |
| | | 977) | (SEQ ID | 1003) | YC (SEQ ID | NO: | ID |
| | | | NO: | | NO: 946) | 1420) | NO: |
| | | | 1017) | | | | 421) |
| BP003T3P2-7 | EVQLQQSGAELVRP | GYTFT | MHWV | ISTYSG | IYNQKFKGK | ARGVT | WGQ |
| | GVSLKISCKGS (SEQ | DYA | RQSHA | DA | ATMTVDKSS | FDY | GTTL |
| | ID NO: 940) | (SEQ ID | KSLEW | (SEQ ID | STAYLELAR | (SEQ ID | TVSS |
| | | NO: 19) | IGV | NO: 21) | LTSDDSAIY | NO: 23) | (SEQ |
| | | | (SEQ ID | | YC (SEQ ID | | ID |
| | | | NO: | | NO: 1033) | | NO: |
| | | | 1128) | | | | 425) |
| BP003T3P2-70 | QAYLQQSGAELVRS | GFNIK | IHWVK | IDPDN | EYAPKFGKA | TVFWY | WGQ |
| | GASVKLSCTAS (SEQ | DYY | QRPEQ | GET | TMTADTSSN | GNNYA | GTTV |
| | ID NO: 1197) | (SEQ ID | GLEWI | (SEQ ID | TAHLQLSSL | GFAY | SA |
| | | NO: | GW | NO: | TSEDTAVYY | (SEQ ID | (SEQ |
| | | 977) | (SEQ ID | 1003) | C (SEQ ID | NO: | ID |
| | | | NO: | | NO: 945) | 1420) | NO: |
| | | | 1017) | | | | 1453) |
| BP003T3P2-71 | QAYLQQSGAELVRS | GFNIK | IHWVK | IDPDN | EYAPKFGKA | TVFWY | WGQ |
| | GASVKLSCTAS (SEQ | DYY | QRPEQ | GET | TMTADTSSN | GNNYA | GTTV |
| | ID NO: 1197) | (SEQ ID | GLEWI | (SEQ ID | TAHLQLSSL | GFAY | SA |
| | | NO: | GW | NO: | TSEDTAVYY | (SEQ ID | (SEQ |
| | | 977) | (SEQ ID | 1003) | C (SEQ ID | NO: | ID |
| | | | NO: | | NO: 945) | 1420) | NO: |
| | | | 1017) | | | | 1453) |
| BP003T3P2-72 | QVQLQQSGAELVRS | GFNIK | IHWVK | IDPDN | EYAPKFGKA | TVFWY | WGQ |
| | GASVKLSCTAS (SEQ | DYY | QRPEQ | GET | TMTADTSSN | GNNYA | GTTV |
| | ID NO: 1339) | (SEQ ID | GLEWI | (SEQ ID | TAHLQLSSL | GFAY | SA |
| | | NO: | GW | NO: | TSEDTAVYY | (SEQ ID | (SEQ |
| | | 977) | (SEQ ID | 1003) | C (SEQ ID | NO: | ID |
| | | | NO: | | NO: 945) | 1420) | NO: |
| | | | 1017) | | | | 1453) |
| BP003T3P2-73 | EVRLQQSGAELVRS | GFNIK | IHWVK | IDPDN | EYAPKFGKA | TVFWY | WGQ |
| | GASVKLPCTAS (SEQ | DYY | QRPEQ | GET | TMTADTSSN | GNNYA | GTTV |
| | ID NO: 942) | (SEQ ID | GLEWI | (SEQ ID | TAHLQLSSL | GFAY | SA |
| | | NO: | GW | NO: | TSEDTAVYY | (SEQ ID | (SEQ |
| | | 977) | (SEQ ID | 1003) | C (SEQ ID | NO: | ID |
| | | | NO: | | NO: 945) | 1420) | NO: |
| | | | 1017) | | | | 1453) |
| BP003T3P2-74 | EVLLQQFGAELVRSG | GFNIK | IHWVK | IDPDN | EYAPKFGKA | TVFWY | WGQ |
| | ASVKLSCTAS (SEQ | DYY | QRPEQ | GET | TMTADTSSN | GNNYA | GTTV |
| | ID NO: 937) | (SEQ ID | GLEWI | (SEQ ID | TAHLQLSSL | GFAY | SA |
| | | NO: | GW | NO: | TSEDTAVYY | (SEQ ID | (SEQ |
| | | 977) | (SEQ ID | 1003) | C (SEQ ID | NO: | ID |
| | | | NO: | | NO: 945) | 1420) | NO: |
| | | | 1017) | | | | 1453) |
| BP003T3P2-75 | QIQLQQSGAELVRPG | GYTFT | MHWV | ISTYSG | SYNQKFKG | ARGVT | WGQ |
| | VSVKISCKGS (SEQ | DYA | KQSHA | DV | KATMTVDK | FDS | GTTV |
| | ID NO: 1240) | (SEQ ID | KSLEW | (SEQ ID | SSSTAYMEL | (SEQ ID | TVSS |
| | | NO: 19) | IGV | NO: 46) | ARLTSEDSAI | NO: 48) | (SEQ |
| | | | (SEQ ID | | YYC (SEQ ID | | ID |
| | | | NO: | | NO: 1389) | | NO: |
| | | | 1126) | | | | 422) |
| BP003T3P2-8 | EVQLQQSGAELVRP | GYTFT | MHWV | ISTYSG | IYNQKFKGK | ARGVT | WGQ |
| | GVSLKISCKGS (SEQ | DYA | RQSHA | DA | ATMTVDKSS | FDY | GTTL |
| | ID NO: 940) | (SEQ ID | KSLEW | (SEQ ID | STAYLELAR | (SEQ ID | TVSS |
| | | NO: 19) | IGV | NO: 21) | LTSDDSAIY | NO: 23) | (SEQ |
| | | | (SEQ ID | | YC (SEQ ID | | ID |
| | | | NO: | | NO: 1033) | | NO: |
| | | | 1128) | | | | 425) |
| BP003T3P2-9 | QIQLQQSGAELVRPG | GYTFT | MHWV | ISTYSG | IYNQKFKGK | ARGVT | WGQ |
| | VSLKISCKGS (SEQ | DYA | RQSHA | DA | ATMTVDKSS | FDY | GTTL |
| | ID NO: 1239) | (SEQ ID | KSLEW | (SEQ ID | STAYLELAR | (SEQ ID | TVSS |
| | | NO: 19) | IGV | NO: 21) | LTSDDSAIY | NO: 23) | (SEQ |
| | | | (SEQ ID | | YC (SEQ ID | | ID |
| | | | NO: | | NO: 1033) | | NO: |
| | | | 1128) | | | | 425) |

TABLE 4B
Exemplary Clones-Light Chain Sequences

| ID | LFR1 | CDRL1 | LFR2 | CDRL2 | LFR3 | CDRL3 | LFR4 |
|---|---|---|---|---|---|---|---|
| BL_592989- | DIQMTQSQKFMSTS | QNVGT | VAWY | SAS | YRYSGVPD | QQYN | FGGGT |
| 2015_G2_P8_G02 | VGDRVSVTCKAS | N (SEQ | QQKPG | (SEQ ID | RFTGSGSG | SYPYT | KLEMK |
| | (SEQ ID NO: 734) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 961) |
| | | | NO: | | ID NO: | | |
| | | | 1425) | | 1483) | | |
| BL_592989- | DVVMTQTPLSLPVS | QSLVH | LHWYL | EVS | NRFSGVPD | SQSTH | FGGGT |
| 2016_H2_P8_H02 | LGDQASISCRSS | SNGNT | QKPGQ | (SEQ ID | RFSGSGSGT | VPYT | KLEIK |
| | (SEQ ID NO: 900) | Y (SEQ | SPKLLI | NO: | DFTLKISRV | (SEQ | (SEQ ID |
| | | ID NO: | Y (SEQ | 944) | EAEDLGVY | ID NO: | NO: |
| | | 1306) | ID NO: | | FC (SEQ ID | 1375) | 959) |
| | | | 1076) | | NO: 1157) | | |
| BL_592989- | DIVMTQSPPTLSLSP | QDVNT | WYQQ | WAS | TRHTGVPS | QQHY | FGGGT |
| 2017_A3_P8_A03 | GERVTLSCKAS | AVA | KPGQA | (SEQ ID | RFSGSGSGT | SSPWT | KLEIK |
| | (SEQ ID NO: 816) | (SEQ ID | PRLLIY | NO: | DFTLTISSL | (SEQ | (SEQ ID |
| | | NO: | (SEQ ID | 1450) | QPEDFATY | ID NO: | NO: |
| | | 1207) | NO: | | YC (SEQ ID | 1258) | 959) |
| | | | 1470) | | NO: 1414) | | |
| BL_592989- | DVVMTQSQKFMST | QNVGT | VAYQQ | SAS | YRYSGVPD | QQYN | FGGGT |
| 2018_B3_P8_B03 | SVGDRVSVTCKAS | N (SEQ | KPGQS | (SEQ ID | RFTGSGSG | SYPYT | KLEIK |
| | (SEQ ID NO: 894) | ID NO: | PKALIY | NO: 13) | TDFTLTISN | (SEQ | (SEQ ID |
| | | 12) | (SEQ ID | | VQSEDLAE | ID NO: | NO: |
| | | | NO: | | YFC (SEQ | 15) | 959) |
| | | | 1430) | | ID NO: | | |
| | | | | | 1483) | | |
| BL_592989- | DVVMTQSQKFMST | QNVGT | VAYQQ | SAS | YRYSGVPD | QQYN | FGGGT |
| 2019_C3_P8_C03 | SVGDRVSVTCKAS | N (SEQ | KPGQS | (SEQ ID | RFTGSGSG | SYPYT | KLEIK |
| | (SEQ ID NO: 894) | ID NO: | PKALIY | NO: 13) | TDFTLTISN | (SEQ | (SEQ ID |
| | | 12) | (SEQ ID | | VQSEDLAE | ID NO: | NO: |
| | | | NO: | | YFC (SEQ | 15) | 959) |
| | | | 1430) | | ID NO: | | |
| | | | | | 1483) | | |
| BL_592989- | DIVLTQSQKFMSTS | QNVGT | VAWY | SAS | YRYSGVPD | QQYN | FGGGT |
| 2021_E3_P8_E03 | VGDRVSVTCKAS | N (SEQ | QQKPG | (SEQ ID | RFTGSGSG | SYPYT | KLEIK |
| | (SEQ ID NO: 773) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1483) | | |
| BL_592989- | DVVMTQTQKFMST | QNVGT | VAWY | SAS | YRYSGVPD | QQYN | FGGGT |
| 2022_F3_P8_F03 | SVGDRVSVTCKAS | N (SEQ | QQKPG | (SEQ ID | RFTGSGSG | SYPYT | KLEIK |
| | (SEQ ID NO: 909) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1483) | | |
| BL_592989- | DVVMTQTPLSLPVS | QSLVH | LHWYL | EVS | NRFSGVPD | SQSTH | FGGGT |
| 2023_G3_P8_G03 | LGDQASISCRSS | SNGNT | QKPGQ | (SEQ ID | RFSGSGSGT | VPYT | KLEIK |
| | (SEQ ID NO: 900) | Y (SEQ | SPKLLI | NO: | DFTLKISRV | (SEQ | (SEQ ID |
| | | ID NO: | Y (SEQ | 944) | EAEDLGVY | ID NO: | NO: |
| | | 1306) | ID NO: | | FC (SEQ ID | 1375) | 959) |
| | | | 1076) | | NO: 1157) | | |
| BL_592989- | DIVMTQTTSSLSASL | QDIRN | LNYQQ | YTS | RLYSGVPS | QQGN | FGGGT |
| 2024_H3_P8_H03 | GDRVTISCRAS (SEQ | Y (SEQ | KPDGT | (SEQ ID | RFSGSGSGT | TLPPT | KLEIK |
| | ID NO: 831) | ID NO: | VKLLIY | NO: | DYSLTISNL | (SEQ | (SEQ ID |
| | | 1202) | (SEQ ID | 1495) | EQEDIATYF | ID NO: | NO: |
| | | | NO: | | C (SEQ ID | 1253) | 959) |
| | | | 1091) | | NO: 1359) | | |
| BL_592989- | DIQMTQSQKFMSTS | QNVGT | VAYQQ | SAS | YRYSGVPD | QQYN | FGGGT |
| 2025_A4_P8_A04 | VGDRVSVTCKAS | N (SEQ | KPGQS | (SEQ ID | RFTGSGSG | SYPYT | KLEIK |
| | (SEQ ID NO: 734) | ID NO: | PKALIY | NO: 13) | TDFTLTISN | (SEQ | (SEQ ID |
| | | 12) | (SEQ ID | | VQSEDLAE | ID NO: | NO: |
| | | | NO: | | YFC (SEQ | 15) | 959) |
| | | | 1430) | | ID NO: | | |
| | | | | | 1483) | | |
| BL_592989- | DIQMTQSPASLSAS | ENIYSY | LEWYQ | NAK | TLAEGVPS | QHHY | FGGGT |
| 2026_B4_P8_B04 | VGESVTITCRAS | (SEQ ID | QKQGK | (SEQ ID | RFSGSGSGT | GSPYT | KLEIK |
| | (SEQ ID NO: 718) | NO: 930) | SPQLL | NO: 28) | QFSLRINSL | (SEQ | (SEQ ID |
| | | | VY | | QPEDFGTY | ID NO: | NO: |
| | | | (SEQ ID | | YC (SEQ ID | 1216) | 959) |
| | | | NO: | | NO: 1400) | | |
| | | | 1072) | | | | |
| BL_592989- | DIQMTQSQKFMSAS | QNVGT | VAYQQ | SAS | YRYSGVPD | QQYN | FGGGT |
| 2027_C4_P8_C04 | VGDRVSVTCKAS | N (SEQ | KPGQS | (SEQ ID | RFTGSGSG | SYPYT | KLEMK |
| | (SEQ ID NO: 729) | ID NO: | PKALIY | NO: 13) | TDFTLTISN | (SEQ | (SEQ ID |
| | | 12) | (SEQ ID | | VQSEDLAE | ID NO: | NO: |
| | | | NO: | | YFC (SEQ | 15) | 961) |
| | | | 1430) | | ID NO: | | |
| | | | | | 1483) | | |
| BL_592989- | DNVLTQSQKFMSTS | QNVGT | VAWY | SAS | YRYSGVPD | QQYN | FGGGT |
| 2029_E4_P8_E04 | VGDRVSVTCKAS | N (SEQ | QQKPG | (SEQ ID | RFTGSGSG | SYPYT | KLEMK |
| | (SEQ ID NO: 837) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 961) |
| | | | NO: | | ID NO: | | |
| | | | 1425) | | 1483) | | |
| BL_592989- | DIVLTQSQKFMSTS | QNVGT | VAWY | SAS | YRYSGVPD | QQYN | FGGGT |
| 2030_F4_P8_F04 | VGDRVSVTCKAS | N (SEQ | QQKPG | (SEQ ID | RFTGSGSG | SYPW | KLEIK |
| | (SEQ ID NO: 773) | ID NO: | QSPKA | NO: 13) | TDFTLTISN | T (SEQ | (SEQ ID |
| | | 12) | LIY | | VQSEDLAE | ID NO: | NO: |
| | | | (SEQ ID | | YFC (SEQ | 36) | 959) |
| | | | NO: | | ID NO: | | |
| | | | 1425) | | 1483) | | |
| BL_592989- | DIQMTQSQKFMSTS | QNVGT | VAWY | SAS | YRYSGVPD | QQYN | FGGGT |
| 2031_G4_P8_G04 | VGDRVSVTCKAS | N (SEQ | QQKPG | (SEQ ID | RFTGSGSG | SYPYT | KLEMK |
| | (SEQ ID NO: 734) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 961) |
| | | | NO: | | ID NO: | | |
| | | | 1425) | | 1483) | | |
| BL_592989- | DVVMTQSQKFMST | QNVGT | VAWY | SAS | YRYSGVPG | QQYN | FGGGT |
| 2032_H4_P8_H04 | SVGDRVSVTCKAS | N (SEQ | QQKPG | (SEQ ID | RFTGSGSG | SYPYT | KLEIK |
| | (SEQ ID NO: 894) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1490) | | |
| BL_592989- | DIQMTQTPASLSAS | ENIYSY | LEWFQ | NAK | TLAEGVPS | QHHY | FGGGT |
| 2033_A5_P8_A05 | VGETVTITCRAS | (SEQ ID | QKQGK | (SEQ ID | RFSVSGSGT | GIPYT | KLEIK |
| | (SEQ ID NO: 744) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 1214) | 959) |
| | | | NO: | | NO: 1401) | | |
| | | | 1070) | | | | |
| BL_592989- | DIQMTQSQKFMSTS | QNVGT | VAYQQ | SAS | YRYSGVPD | QQYN | FGGGT |
| 2034_B5_P8_B05 | VGDRVSVTCKAS | N (SEQ | KPGQS | (SEQ ID | RFTGSGSG | SYPYT | KLEIK |
| | (SEQ ID NO: 734) | ID NO: | PKALIY | NO: 13) | TDFTLTISN | (SEQ | (SEQ ID |
| | | 12) | (SEQ ID | | VQSEDLAE | ID NO: | NO: |
| | | | NO: | | YFC (SEQ | 15) | 959) |
| | | | 1430) | | ID NO: | | |
| | | | | | 1483) | | |
| BL_592989- | DIVMTQSQKFMSTS | QNVGT | VAYQQ | SAS | YRYSGVPD | QQYN | FGGGT |
| 2035_C5_P8_C05 | VGDRVSVTCKAS | N (SEQ | KPGQS | (SEQ ID | RFTGSGSG | SYPYT | KLEIK |
| | (SEQ ID NO: 821) | ID NO: | PKALIY | NO: 13) | TDFTLTISN | (SEQ | (SEQ ID |
| | | 12) | (SEQ ID | | VQSEDLAE | ID NO: | NO: |
| | | | NO: | | YFC (SEQ | 15) | 959) |
| | | | 1430) | | ID NO: 1483) | | |
| BL_592989- | DIVMSQSQKFMSTS | QNVGT | VAWY | SAS | YRYSGVPD | QQYN | FGGGT |
| 2037_E5_P8_E05 | VGDRVSVTCKAS | N (SEQ | QQKPG | (SEQ ID | RFTGSGSG | SYPYT | KLEIK |
| | (SEQ ID NO: 793) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1483) | | |
| BL_592989- | DIVMTQSQKFMSTS | QNVGT | VAWY | SAS | YRYSGVPD | QQYN | FGGGT |
| 2038_F5_P8_F05 | VGDRVSVTCKAS | N (SEQ | QQKPG | (SEQ ID | RFTGSGSG | SYPYT | KLEMK |
| | (SEQ ID NO: 821) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 961) |
| | | | NO: | | ID NO: | | |
| | | | 1425) | | 1483) | | |
| BL_592989- | DIVMSQSQKFMSTS | QNVGT | VAWY | SAS | YRYSGVPD | QQYN | FGGGT |
| 2039_G5_P8_G05 | VGDRVSVTCKAS | N (SEQ | QQKPG | (SEQ ID | RFTGSGSG | NYPW | KLEIK |
| | (SEQ ID NO: 793) | ID NO: | QSPKA | NO: 13) | TDFTLTISN | T (SEQ | (SEQ ID |
| | | 12) | LIY | | VQSEDLAE | ID NO: | NO: |
| | | | (SEQ ID | | YFC (SEQ | 1284) | 959) |
| | | | NO: | | ID NO: | | |
| | | | 1425) | | 1483) | | |
| BL_592989- | DIQMTQSQKFMSTS | QNVGT | VAWY | SAS | YRYSGVPD | QQYN | FGGGT |
| 2040_H5_P8_H05 | VGDRVSVTCKAS | N (SEQ | QQKPG | (SEQ ID | RFTGSGSG | SYPYT | KLEIK |
| | (SEQ ID NO: 734) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1483) | | |
| BL_592989- | DVVMTQSPASLSAS | ENSYS | LEWYQ | NAK | TLAEGVPS | QHHY | FGGGT |
| 2041_A6_P8_A06 | VGETVTITCRAS | Y (SEQ | QKQGK | (SEQ ID | RFSGSGSGT | GTPYT | KLEIK |
| | (SEQ ID NO: 890) | ID NO: | SPQLL | NO: 28) | QFSLKINSL | (SEQ | (SEQ ID |
| | | 52) | VY | | QPEDFGTY | ID NO: | NO: |
| | | | (SEQ ID | | YC (SEQ ID | 54) | 959) |
| | | | NO: | | NO: 1399) | | |
| | | | 1072) | | | | |
| BL_592989- | DIQMTQSPASLSAS | ENSYS | LEWYQ | NAK | TLAEGVPS | QHHY | FGGGT |
| 2042_B6_P8_B06 | VGETVTITCRAS | Y (SEQ | QKQGK | (SEQ ID | RFSGSGSGT | GTPYT | KLEIK |
| | (SEQ ID NO: 720) | ID NO: | SPQLL | NO: 28) | QFSLKINSL | (SEQ | (SEQ ID |
| | | 52) | VY | | QPEDFGTY | ID NO: | NO: |
| | | | (SEQ ID | | YC (SEQ ID | 54) | 959) |
| | | | NO: | | NO: 1399) | | |
| | | | 1072) | | | | |
| BL_592989- | DIQMTQSPASLSAS | ENIYSY | LEWYQ | NAK | TLAEGVPS | QHHY | FGGGT |
| 2043_C6_P8_C06 | VGETVTITCRAS | (SEQ ID | QKQGK | (SEQ ID | RFSGSGSGT | GTPYT | KLEIK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 54) | 959) |
| | | | NO: | | NO: 1398) | | |
| | | | 1072) | | | | |
| BL_592989- | DIVLTQSQKFMSTS | QNVGT | VAYQQ | SAS | YRYSGVPD | QQYN | FGGGT |
| 2044_D6_P8_D06 | VGDRVSVTCKAS | N (SEQ | KPGQS | (SEQ ID | RFTGSGSG | TYPYT | KLEMK |
| | (SEQ ID NO: 773) | ID NO: | PKALIY | NO: 13) | TDFTLTISN | (SEQ | (SEQ ID |
| | | 12) | (SEQ ID | | VQSEDLAE | ID NO: | NO: |
| | | | NO: | | YFC (SEQ | 1287) | 961) |
| | | | 1430) | | ID NO: 1483) | | |
| BL_592989- | DIVMSQSQKFMSTS | QNVGT | VAWY | SAS | YRYSGVPD | QQYN | FGGGT |
| 2045_E6_P8_E06 | VGDRVSVTCKAS | N (SEQ | QQKPG | (SEQ ID | RFTGSGSG | SYPYT | KLEIK |
| | (SEQ ID NO: 793) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1483) | | |
| BL_592989- | DIQMTQSQKFMSTS | QNVGT | VAWY | SAS | YRYSGVPD | QQYN | FGGGT |
| 2046_F6_P8_F06 | VGDRVSVTCKAS | N (SEQ | QQKPG | (SEQ ID | RFTGSGSG | SYPYT | KLEIK |
| | (SEQ ID NO: 734) | ID NO: | QSPKA | NO: 13) | TGFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1487) | | |
| BL_592989- | DIVMSQSQKFMSTS | QNVGT | VAWY | SAS | YRYSGVPD | QQYN | FGGGT |
| 2047_G6_P8_G06 | VGDRVSVTCKAS | N (SEQ | QQKPG | (SEQ ID | RFTGSGSG | NYPW | KLEIK |
| | (SEQ ID NO: 793) | ID NO: | QSPKA | NO: 13) | TDFTLTISN | T (SEQ | (SEQ ID |
| | | 12) | LIY | | VQSEDLAE | ID NO: | NO: |
| | | | (SEQ ID | | YFC (SEQ | 1284) | 959) |
| | | | NO: | | ID NO: | | |
| | | | 1425) | | 1483) | | |
| BL_592989- | DIVMSQSQKFMSTS | QNVGT | VAWY | SAS | YRYSGVPD | QQYN | FGGGT |
| 2048_H6_P8_H06 | VGDRVSVTCKAS | N (SEQ | QQKPG | (SEQ ID | RFTGSGSG | SYPYT | KLEIK |
| | (SEQ ID NO: 793) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1483) | | |
| BL_592989- | DIQMTQSPASLSAS | ENIYSY | LEWYQ | NAK | TLAEGVPS | QHHY | FGGGT |
| 2049_A7_P8_A07 | VGETVTITCRAS | (SEQ ID | QKQGK | (SEQ ID | RFSGSGSGT | GTPYT | KLEIK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 54) | 959) |
| | | | NO: | | NO: 1398) | | |
| | | | 1072) | | | | |
| BL_592989- | DIQMTQSQKFMSTS | QNVGT | VAYQQ | SAS | YRYSGVPD | QQYN | FGGGT |
| 2051_C7_P8_C07 | VGDRVSVTCKAS | N (SEQ | KPGQS | (SEQ ID | RFTGSGSG | SYPYT | KLEIK |
| | (SEQ ID NO: 734) | ID NO: | PKALIY | NO: 13) | TDFTLTISN | (SEQ | (SEQ ID |
| | | 12) | (SEQ ID | | VQSEDLAE | ID NO: | NO: |
| | | | NO: | | YFC (SEQ | 15) | 959) |
| | | | 1430) | | ID NO: | | |
| | | | | | 1483) | | |
| BL_592989- | DVVLTQTPLSLPVSL | QSIVHS | LEWYL | KVS | NRFSGVPD | FQGSH | FGGGT |
| 2052_D7_P8_D07 | GDQASISCRSS (SEQ | NGNTY | QKPGQ | (SEQ ID | RFSGSGSGT | VPPT | KLEIK |
| | ID NO: 874) | (SEQ ID | SPKLLI | NO: | DFTLKISKV | (SEQ | (SEQ ID |
| | | NO: | Y (SEQ | 1062) | EAEDLGVY | ID NO: | NO: |
| | | 1299) | ID NO: | | YC (SEQ ID | 966) | 959) |
| | | | 1071) | | NO: 1154) | | |
| BL_592989- | DTTVTQSQKFMSTS | QNVGT | VAWY | SAS | YRYSGVPD | QQYN | FGGGT |
| 2054_F7_P8_F07 | VGDRVSVTCKAS | N (SEQ | QQKPG | (SEQ ID | RFTGSGSG | NHPYT | KLEIK |
| | (SEQ ID NO: 858) | ID NO: | QSPKPL | NO: 13) | TDFTLTISN | (SEQ | (SEQ ID |
| | | 12) | IY (SEQ | | VQSEDLAE | ID NO: | NO: |
| | | | ID NO: | | YFC (SEQ | 1281) | 959) |
| | | | 1429) | | ID NO: | | |
| | | | | | 1483) | | |
| BL_592989- | DIQMTQSQKFMSTS | QNVGT | VAWY | SAS | YRYSGVPD | QQYN | FGGGT |
| 2055_G7_P8_G07 | VGDRASVTCKAS | N (SEQ | QQKPG | (SEQ ID | RFTGSGSG | SYPW | KLEIK |
| | (SEQ ID NO: 731) | ID NO: | QSPKA | NO: 13) | TDFTLTISN | T (SEQ | (SEQ ID |
| | | 12) | LIY | | VQSEDLAE | ID NO: | NO: |
| | | | (SEQ ID | | YFC (SEQ | 36) | 959) |
| | | | NO: | | ID NO: | | |
| | | | 1425) | | 1483) | | |
| BL_592989- | DIVMTQSQKFMSTS | QNVGT | VAYQQ | SAS | YRYSGVPD | QQYN | FGGGT |
| 2059_C8_P8_C08 | VGDRVSVTCKAS | N (SEQ | KPGQS | (SEQ ID | RFTGSGSG | TYPYT | KLEIK |
| | (SEQ ID NO: 821) | ID NO: | PKALIY | NO: 13) | TDFTLTISN | (SEQ | (SEQ ID |
| | | 12) | (SEQ ID | | VQSEDLAD | ID NO: | NO: |
| | | | NO: | | YFC (SEQ | 1287) | 959) |
| | | | 1430) | | ID NO: | | |
| | | | | | 1482) | | |
| BL_592989- | DIQMTQSPASLSAS | ENIYSY | LEWYQ | NAK | TLAEGVPS | QHHY | FGGGT |
| 2060_D8_P8_D08 | VGESVTITCRAS | (SEQ ID | QKQGK | (SEQ ID | RFSGSGSGT | GSPYT | KLEMK |
| | (SEQ ID NO: 718) | NO: 930) | SPQLL | NO: 28) | QFSLRINSL | (SEQ | (SEQ ID |
| | | | VY | | QPEDFGTY | ID NO: | NO: |
| | | | (SEQ ID | | YC (SEQ ID | 1216) | 961) |
| | | | NO: | | NO: 1400) | | |
| | | | 1072) | | | | |
| BL_592989- | DTTVTQSQKFMSTS | QNVGT | VAWY | SAS | YRYGGVPD | QQYN | FGGGT |
| 2061_E8_P8_E08 | VGDRVSVTCKAS | N (SEQ | QQKPG | (SEQ ID | RFTGSGSG | SYPYT | KLEIK |
| | (SEQ ID NO: 858) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1477) | | |
| BL_592989- | DIVMTQSPASLSAS | ENIYSF | LVWYQ | NAK | TLAEGVPS | QHHY | FGGGT |
| 2062_F8_P8_F08 | VGETVTITCRAS | (SEQ ID | QKQGK | (SEQ ID | RFSGIGSGT | GTPYT | KLEIK |
| | (SEQ ID NO: 807) | NO: 928) | SPHLL | NO: 28) | QFSLKINSL | (SEQ | (SEQ ID |
| | | | VY | | QPEDFGTY | ID NO: | NO: |
| | | | (SEQ ID | | YC (SEQ ID | 54) | 959) |
| | | | NO: | | NO: 1396) | | |
| | | | 1117) | | | | |
| BL_592989- | DIQMTQSQKFMSTS | QNVGT | WYQQ | SAS | YRYSGVPD | QQYN | FGGGT |
| 2063_G8_P8_G08 | VGDRVGVTCKAS | NVA | KPGQS | (SEQ ID | RFTGSGSG | SYPYT | KLEMK |
| | (SEQ ID NO: 732) | (SEQ ID | PKALIY | NO: 13) | TDFTLTISN | (SEQ | (SEQ ID |
| | | NO: | (SEQ ID | | VQSEDLAE | ID NO: | NO: |
| | | 1246) | NO: | | YFC (SEQ | 15) | 961) |
| | | | 1471) | | ID NO: | | |
| | | | | | 1483) | | |
| BL_592989- | DTTVTQSQKFMSTS | QNVGT | VAWY | SAS | YRYSGVPD | QQYN | FGGGT |
| 2064_H8_P8_H08 | VGDRVSVTCKAS | N (SEQ | QQKPG | (SEQ ID | RFTGSGSG | SYPW | KLEIK |
| | (SEQ ID NO: 858) | ID NO: | QSPKA | NO: 13) | TDFTLTISN | T (SEQ | (SEQ ID |
| | | 12) | LIY | | VQSEDLAE | ID NO: | NO: |
| | | | (SEQ ID | | YFC (SEQ | 36) | 959) |
| | | | NO: | | ID NO: | | |
| | | | 1425) | | 1483) | | |
| BL_592989- | DIVMTQSPASLSAS | ENIYSF | LVWYQ | NAK | TLAEGVPS | QHHY | FGGGT |
| 2065_A9_P8_A09 | VGETVTITCRAS | (SEQ ID | QKQGK | (SEQ ID | RFSGIGSGT | GTPYT | KLEIK |
| | (SEQ ID NO: 807) | NO: 928) | SPHLL | NO: 28) | QFSLKINSL | (SEQ | (SEQ ID |
| | | | VY | | QPEDFGTY | ID NO: | NO: |
| | | | (SEQ ID | | YC (SEQ ID | 54) | 959) |
| | | | NO: | | NO: 1396) | | |
| | | | 1117) | | | | |
| BL_592989- | DILMTQSQKFMSTS | QNVGT | VAYQQ | SAS | YRYSGVPD | QQYN | FGGGT |
| 2067_C9_P8_C09 | VGDRVSVTCKAS | N (SEQ | KPGQS | (SEQ ID | RFTGSGSG | NYPLT | KLEIK |
| | (SEQ ID NO: 711) | ID NO: | PKPLIY | NO: 13) | TDFTLTISN | (SEQ | (SEQ ID |
| | | 12) | (SEQ ID | | VQSEDLAE | ID NO: | NO: |
| | | | NO: | | YFC (SEQ | 1283) | 959) |
| | | | 1432) | | ID NO: | | |
| | | | | | 1483) | | |
| BL_592989- | DIQMTQSPASLSAS | ENIYSY | LEWYQ | NAK | TLVEGVPS | QHHY | FGGGT |
| 2068_D9_P8_D09 | VGETVTITCRAS | (SEQ ID | QKQGK | (SEQ ID | RFSGSGSGT | DTPYT | KLEIK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 30) | 959) |
| | | | NO: | | NO: 1408) | | |
| | | | 1072) | | | | |
| BL_592989- | DTTVTQSQKFMSTS | QNVGT | VAWY | SAS | YRYSGVPD | QQYN | FGGGT |
| 2069_E9_P8_E09 | VGDRVSVTCKAS | N (SEQ | QQKPG | (SEQ ID | RFTGSGSG | SYPYT | KLEIK |
| | (SEQ ID NO: 858) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1483) | | |
| BL_592989- | DVVMTQSPASLSAS | ENIYSY | LEWYQ | NAK | TLAEGVPS | QHHY | FGGGT |
| 2070_F9_P8_F09 | VGESVTITCRAS | (SEQ ID | QKQGK | (SEQ ID | RFSGSGSGT | GSPYT | KLEIK |
| | (SEQ ID NO: 889) | NO: 930) | SPQLL | NO: 28) | QFSLRINSL | (SEQ | (SEQ ID |
| | | | VY | | QPEDFGTY | ID NO: | NO: |
| | | | (SEQ ID | | YC (SEQ ID | 1216) | 959) |
| | | | NO: | | NO: 1400) | | |
| | | | 1072) | | | | |
| BL_592989- | DTTVTQSQKFMSTS | QNVGT | VAWY | SAS | YRYSGVPD | QQYN | FGGGT |
| 2071_G9_P8_G09 | VGDRVSVACKAS | N (SEQ | QQKPG | (SEQ ID | RFTGSGSG | SYPYT | KLEIK |
| | (SEQ ID NO: 854) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1483) | | |
| BL_592989- | DTTVTQSQKFMSTS | QNVGT | VAYQQ | SAS | YRYSGVPD | QQYN | FGGGT |
| 2074_B10_P8_B10 | VGDRVSVTCKAS | N (SEQ | KPGQS | (SEQ ID | RFTGSGSG | SYPYT | KLEMK |
| | (SEQ ID NO: 858) | ID NO: | PKALIY | NO: 13) | TDFTLTISN | (SEQ | (SEQ ID |
| | | 12) | (SEQ ID | | VQSEDLAE | ID NO: | NO: |
| | | | NO: | | YFC (SEQ | 15) | 961) |
| | | | 1430) | | ID NO: | | |
| | | | | | 1483) | | |
| BL_592989- | DIVITQSQKFMSTSV | QNVGT | VAYQQ | SAS | YRYSGVPD | QQYN | FGGGT |
| 2076_D10_P8_D10 | GDRVSVTCKAS | N (SEQ | KPGQS | (SEQ ID | RLTGSGSG | RYPYT | KLEIK |
| | (SEQ ID NO: 766) | ID NO: | PKALIY | NO: 13) | TDFTLTISN | (SEQ | (SEQ ID |
| | | 12) | (SEQ ID | | VQSEDLAE | ID NO: | NO: |
| | | | NO: | | YFC (SEQ | 1285) | 959) |
| | | | 1430) | | ID NO: | | |
| | | | | | 1489) | | |
| BL_592989- | DIQMTQTQKFMSTS | QNVGT | VAWY | SAS | YRYSGVPD | QQYN | FGGGT |
| 2077_E10_P8_E10 | VGDRVSVTCKAS | N (SEQ | QQKPG | (SEQ ID | RFTGSGSG | SYPW | KLEIK |
| | (SEQ ID NO: 757) | ID NO: | QSPKA | NO: 13) | TDFTLTISN | T (SEQ | (SEQ ID |
| | | 12) | LIY | | VQSEDLTE | ID NO: | NO: |
| | | | (SEQ ID | | YFC (SEQ | 36) | 959) |
| | | | NO: | | ID NO: | | |
| | | | 1425) | | 1486) | | |
| BL_592989- | DTTVTQSQKFMSTS | QNVGT | VAWY | SAS | YRYSGVPD | QQYN | FGGGT |
| 2078_F10_P8_F10 | VGDRVSVTCKAS | N (SEQ | QQKPG | (SEQ ID | RFTGSGSG | SYPYT | KLEMK |
| | (SEQ ID NO: 858) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 961) |
| | | | NO: | | ID NO: | | |
| | | | 1425) | | 1483) | | |
| BL_592989- | DIQMTQSQKFMSTS | QNVGT | VAWY | SAS | YRYSGVPD | QQYN | FGGGT |
| 2079_G10_P8_G10 | VGDRVSVTCKAS | N (SEQ | QQKPG | (SEQ ID | RFTGSGSG | SYPW | KLEMK |
| | (SEQ ID NO: 734) | ID NO: | QSPKA | NO: 13) | TDFTLTISN | T (SEQ | (SEQ ID |
| | | 12) | LIY | | VQSEDLAE | ID NO: | NO: |
| | | | (SEQ ID | | YFC (SEQ | 36) | 961) |
| | | | NO: | | ID NO: | | |
| | | | 1425) | | 1483) | | |
| BL_592989- | DIQMTQSTSSLSASL | QDIRN | LNYQQ | YTS | RLHSGVPS | QQGN | FGGGT |
| 2080_H10_P8_H10 | GDRVTISCRAS (SEQ | Y (SEQ | KPDGT | (SEQ ID | RFSGSGSGT | TLPPT | KLEIK |
| | ID NO: 738) | ID NO: | VKLLIY | NO: | DYSLTISNL | (SEQ | (SEQ ID |
| | | 1202) | (SEQ ID | 1495) | EQEDIATYF | ID NO: | NO: |
| | | | NO: | | C (SEQ ID | 1253) | 959) |
| | | | 1091) | | NO: 1353) | | |
| BP003-T2P1A7 | DIQMTQSPASLSAS | ENIYSY | LEWYQ | NAK | TLAEGVPS | QHHY | FGGGT |
| | VGETVTITCRAS | (SEQ ID | QKQGK | (SEQ ID | RFSGSGSGT | GTPYT | KLEIK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 54) | 959) |
| | | | NO: | | NO: 1398) | | |
| | | | 1072) | | | | |
| BP003-T2P1C8 | DIVMTQSQKFMSTS | QNVGT | VAWY | SAS | YRYSGVPD | QQYN | FGGGT |
| | VGDRVSVTCKAS | N (SEQ | QQKPG | (SEQ ID | RFTGSGSG | TYPYT | KLEIK |
| | (SEQ ID NO: 821) | ID NO: | QSPKA | NO: 13) | TDFTLTISN | (SEQ | (SEQ ID |
| | | 12) | LIY | | VQSEDLAD | ID NO: | NO: |
| | | | (SEQ ID | | YFC (SEQ | 1287) | 959) |
| | | | NO: | | ID NO: | | |
| | | | 1425) | | 1482) | | |
| BP003-T2P1F4 | DIVLTQSQKFMSTS | QNVGT | VAWY | SAS | YRYSGVPD | QQYN | FGGGT |
| | VGDRVSVTCKAS | N (SEQ | QQKPG | (SEQ ID | RFTGSGSG | SYPW | KLEIK |
| | (SEQ ID NO: 773) | ID NO: | QSPKA | NO: 13) | TDFTLTISN | T (SEQ | (SEQ ID |
| | | 12) | LIY | | VQSEDLAE | ID NO: | NO: |
| | | | (SEQ ID | | YFC (SEQ | 36) | 959) |
| | | | NO: | | ID NO: | | |
| | | | 1425) | | 1483) | | |
| BP003-T2P1G2 | DIQMTQSQKFMSTS | QNVGT | VAWY | SAS | YRYSGVPD | QQYN | FGGGT |
| | VGDRVSVTCKAS | N (SEQ | QQKPG | (SEQ ID | RFTGSGSG | SYPYT | KLEMK |
| | (SEQ ID NO: 734) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 961) |
| | | | NO: | | ID NO: | | |
| | | | 1425) | | 1483) | | |
| BP003-T2P1G4 | DIQMTQSQKFMSTS | QNVGT | VAWY | SAS | YRYSGVPD | QQYN | FGGGT |
| | VGDRVSVTCKAS | N (SEQ | QQKPG | (SEQ ID | RFTGSGSG | SYPYT | KLEMK |
| | (SEQ ID NO: 734) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 961) |
| | | | NO: | | ID NO: | | |
| | | | 1425) | | 1483) | | |
| BP003-T2P1G7 | DIQMTQSQKFMSTS | QNVGT | VAWY | SAS | YRYSGVPD | QQYN | FGGGT |
| | VGDRASVTCKAS | N (SEQ | QQKPG | (SEQ ID | RFTGSGSG | SYPW | KLEIK |
| | (SEQ ID NO: 731) | ID NO: | QSPKA | NO: 13) | TDFTLTISN | T (SEQ | (SEQ ID |
| | | 12) | LIY | | VQSEDLAE | ID NO: | NO: |
| | | | (SEQ ID | | YFC (SEQ | 36) | 959) |
| | | | NO: | | ID NO: | | |
| | | | 1425) | | 1483) | | |
| BP003-T2P1H5 | DIQMTQSQKFMSTS | QNVGT | VAWY | SAS | YRYSGVPD | QQYN | FGGGT |
| | VGDRVSVTCKAS | N (SEQ | QQKPG | (SEQ ID | RFTGSGSG | SYPYT | KLEIK |
| | (SEQ ID NO: 734) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1483) | | |
| BP003T3P2-1 | DIQMTQSPASLSAS | ENIYSY | LEWYQ | NAK | TLVEGVPS | QHHY | FGGGT |
| | VGETVTITCRAS | (SEQ ID | QKQGK | (SEQ ID | RFSGSGSGT | DTPYT | KLEIK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 30) | 959) |
| | | | NO: | | NO: 1408) | | |
| | | | 1072) | | | | |
| BP003T3P2-10 | DVQMTQSPSSMYAS | QDINR | LSWFQ | RAN | RLVDGVPS | LQYD | FGGGT |
| | LGERVTFTCKAS | Y (SEQ | QKPGK | (SEQ ID | RFSGSGSG | EFPYT | KLEIK |
| | (SEQ ID NO: 872) | ID NO: | SPKTLI | NO: | QNYSLTISS | (SEQ | (SEQ ID |
| | | 1201) | Y (SEQ | 1345) | LEYEDMGI | ID NO: | NO: |
| | | | ID NO: | | YYC (SEQ | 1106) | 959) |
| | | | 1109) | | ID NO: | | |
| | | | | | 1357) | | |
| BP003T3P2-11 | DVQMTQSPSSMYAS | QDINR | LSWFQ | RAN | RLVDGVPS | LQYD | FGGGT |
| | LGERVTFTCKAS | Y (SEQ | QKPGK | (SEQ ID | RFSGSGSG | EFPYT | KLEIK |
| | (SEQ ID NO: 872) | ID NO: | SPKTLI | NO: | QNYSLTISS | (SEQ | (SEQ ID |
| | | 1201) | Y (SEQ | 1345) | LEYEDMGI | ID NO: | NO: |
| | | | ID NO: | | YYC (SEQ | 1106) | 959) |
| | | | 1109) | | ID NO: | | |
| | | | | | 1357) | | |
| BP003T3P2-12 | DVQMNQSPSSMYA | QDINNF | LSWFQ | RAN | RLVDGVPS | LQYD | FGGGT |
| | SLGERVTITCKAS | (SEQ ID | QKPGK | (SEQ ID | RFSGSGSG | EFPWT | KLEIK |
| | (SEQ ID NO: 870) | NO: | SPQTLI | NO: | QHYSLTISG | (SEQ | (SEQ ID |
| | | 1200) | Y (SEQ | 1345) | LEYEDLGIY | ID NO: | NO: |
| | | | ID NO: | | YC (SEQ ID | 1105) | 959) |
| | | | 1110) | | NO: 1356) | | |
| BP003T3P2-13 | DIQMTQSPASLSAS | ENIYSY | LEWYQ | NAK | TLAEGVPS | QHHY | FGGGT |
| | VGESVTITCRAS | (SEQ ID | QKQGK | (SEQ ID | RFSGSGSGT | GSPYT | KLEIK |
| | (SEQ ID NO: 718) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 1216) | 959) |
| | | | NO: | | NO: 1398) | | |
| | | | 1072) | | | | |
| BP003T3P2-14 | DIQMTQSPASLSAS | ENIYSY | LEWYQ | NAK | TLAEGVPS | QHHY | FGGGT |
| | VGETVTITCRAS | (SEQ ID | QKQGK | (SEQ ID | RFSGSGSGT | DTPYT | KLEIK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 30) | 959) |
| | | | NO: | | NO: 1398) | | |
| | | | 1072) | | | | |
| BP003T3P2-15 | DVVMTQSPASLSAS | ENSYS | LEWYQ | NAK | TLAEGVPS | QHHY | FGGGT |
| | VGETVTITCRAS | Y (SEQ | QKQGK | (SEQ ID | RFSGSGSGT | GTPYT | KLEIK |
| | (SEQ ID NO: 890) | ID NO: | SPQLL | NO: 28) | QFSLKINSL | (SEQ | (SEQ ID |
| | | 52) | VY | | QPEDFGTY | ID NO: | NO: |
| | | | (SEQ ID | | YC (SEQ ID | 54) | 959) |
| | | | NO: | | NO: 1399) | | |
| | | | 1072) | | | | |
| BP003T3P2-16 | DIQMTQSPASLSAS | ENIYSY | LEWYQ | NAK | TLAEGVPS | QHHY | FGGGT |
| | VGETVTITCRAS | (SEQ ID | QKQGK | (SEQ ID | RFSGSGSGT | DTPYT | KLEIK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 30) | 959) |
| | | | NO: | | NO: 1398) | | |
| | | | 1072) | | | | |
| BP003T3P2-17 | DVQMTQSPASLSAS | ENIYSY | LEWYQ | NAK | TLAEGVPS | QHHY | FGGGT |
| | VGETVTITCRAS | (SEQ ID | QKQGK | (SEQ ID | RFSGSGSGT | DTPYT | KLEIK |
| | (SEQ ID NO: 871) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 30) | 959) |
| | | | NO: | | NO: 1398) | | |
| | | | 1072) | | | | |
| BP003T3P2-18 | DIQMTQSPASLSAS | ENIYSY | LEWYQ | NAK | TLAEGVPS | QHHY | FGGGT |
| | VGESVTITCRAS | (SEQ ID | QKQGK | (SEQ ID | RFSGSGSGT | GSPYT | KLEMK |
| | (SEQ ID NO: 718) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 1216) | 961) |
| | | | NO: | | NO: 1398) | | |
| | | | 1072) | | | | |
| BP003T3P2-19 | DIQMTQSPASLSAS | ENIYSY | LEWYQ | NAK | TLAEGVPS | QHHY | FGGGT |
| | VGETVTITCRAS | (SEQ ID | QKQGK | (SEQ ID | RFSGSGSGT | DTPYT | KLEIK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 30) | 959) |
| | | | NO: | | NO: 1398) | | |
| | | | 1072) | | | | |
| BP003T3P2-2 | DIQMTQSPASLSAS | ENIYSY | LEWYQ | NAK | TLVEGVPS | QHHY | FGGGT |
| | VGETVTITCRAS | (SEQ ID | QKQGK | (SEQ ID | RFSGSGSGT | DTPYT | KLEIK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 30) | 959) |
| | | | NO: | | NO: 1408) | | |
| | | | 1072) | | | | |
| BP003T3P2-20 | DIVMTQSPASLSAS | KNSYS | LEWYQ | NAK | TLAEGVPS | QHHY | FGGGT |
| | VGETVTITCRAS | Y (SEQ | QKQGK | (SEQ ID | RFSGSGSGT | GTPYT | KLEIK |
| | (SEQ ID NO: 807) | ID NO: | SPQLL | NO: 28) | QFSLKINSL | (SEQ | (SEQ ID |
| | | 1057) | VY | | QPEDFGTY | ID NO: | NO: |
| | | | (SEQ ID | | YC (SEQ ID | 54) | 959) |
| | | | NO: | | NO: 1399) | | |
| | | | 1072) | | | | |
| BP003T3P2-21 | DIQMTQSPASLSAS | ENIYSY | LEWYQ | NAK | TLAEGVPS | QHHY | FGGGT |
| | VGETVTITCRAS | (SEQ ID | QKQGK | (SEQ ID | RFSGSGSGT | GTPYT | KLEMK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 54) | 961) |
| | | | NO: | | NO: 1398) | | |
| | | | 1072) | | | | |
| BP003T3P2-22 | DIQMTQSPASLSAS | ENSYS | LEWYQ | NAK | TLAEGVPS | QHHY | FGGGT |
| | VGETVTITCRAS | Y (SEQ | QKQGK | (SEQ ID | RFSGSGSGT | GTPYT | KLEIK |
| | (SEQ ID NO: 720) | ID NO: | SPQLL | NO: 28) | QFSLKINSL | (SEQ | (SEQ ID |
| | | 52) | VY | | QPEDFGTY | ID NO: | NO: |
| | | | (SEQ ID | | YC (SEQ ID | 54) | 959) |
| | | | NO: | | NO: 1399) | | |
| | | | 1072) | | | | |
| BP003T3P2-23 | DIQMTQSPASLSAS | ENSYS | LEWYQ | NAK | TLAEGVPS | QHHY | FGGGT |
| | VGESVTITCRAS | Y (SEQ | QKQGK | (SEQ ID | RFSGSGSGT | GSPYT | KLEIK |
| | (SEQ ID NO: 718) | ID NO: | SPQLL | NO: 28) | QFSLRINSL | (SEQ | (SEQ ID |
| | | 52) | VY | | QPEDFGTY | ID NO: | NO: |
| | | | (SEQ ID | | YC (SEQ ID | 1216) | 959) |
| | | | NO: | | NO: 1400) | | |
| | | | 1072) | | | | |
| BP003T3P2-24 | DIQMTQSPASLSAS | ENSYS | LEWYQ | NAK | TLAEGVPS | QHHY | FGGGT |
| | VGESVTITCRAS | Y (SEQ | QKQGK | (SEQ ID | RFSGSGSGT | GSPYT | KLEIK |
| | (SEQ ID NO: 718) | ID NO: | SPQLL | NO: 28) | QFSLRINSL | (SEQ | (SEQ ID |
| | | 52) | VY | | QPEDFGTY | ID NO: | NO: |
| | | | (SEQ ID | | YC (SEQ ID | 1216) | 959) |
| | | | NO: | | NO: 1400) | | |
| | | | 1072) | | | | |
| BP003T3P2-25 | DIQMTQSPASLSAS | ENSYS | LEWYQ | NAK | TLAEGVPS | QHHY | FGGGT |
| | VGESVTITCRTS | Y (SEQ | QKQGK | (SEQ ID | RFSGSGSGT | GSPYT | KLEMK |
| | (SEQ ID NO: 719) | ID NO: | SPQLL | NO: 28) | QFSLRINSL | (SEQ | (SEQ ID |
| | | 52) | VY | | QPEDFGTY | ID NO: | NO: |
| | | | (SEQ ID | | YC | 1216) | 961) |
| | | | NO: | | (SEQ ID | | |
| | | | 1072) | | NO: 1400) | | |
| BP003T3P2-26 | DIVLTQSPASLSASV | ENIYSY | VEWYQ | NAK | TLAEGVPS | QHHY | FGGGT |
| | GESVTITCRAS (SEQ | (SEQ ID | QKQGK | (SEQ ID | RFSGSGSGT | GSPYT | KLEMK |
| | ID NO: 770) | NO: 930) | SPQLL | NO: 28) | HFSLKINSL | (SEQ | (SEQ ID |
| | | | VY | | QPEDFGNY | ID NO: | NO: |
| | | | (SEQ ID | | YC (SEQ ID | 1216) | 961) |
| | | | NO: | | NO: 1397) | | |
| | | | 1433) | | | | |
| BP003T3P2-27 | DIXMTQSTSSLSASL | QDISNY | VNWY | YAS | RLHSGVPS | QQGN | FGGGT |
| | GDRXTITCRAS (SEQ | (SEQ ID | QQKPD | (SEQ ID | RFSGSGSGT | TLPWT | KLEIK |
| | ID NO: 832) | NO: 27) | GTVKL | NO: | DYSLTISNL | (SEQ | (SEQ ID |
| | | | LIY | 1476) | EQEDFANY | ID NO: | NO: |
| | | | (SEQ ID | | FC (SEQ ID | 1255) | 959) |
| | | | NO: | | NO: 1352) | | |
| | | | 1436) | | | | |
| BP003T3P2-28 | DIQMTQTTSSLSASL | QDISNY | LNWYQ | HTS | NLQSGVPS | QQSNT | FGGGT |
| | GDRVTISCRAS (SEQ | (SEQ ID | QKPDG | (SEQ ID | RFTGSGSG | LPPT | KLEIK |
| | ID NO: 760) | NO: 27) | TVKLLI | NO: | TDYSLTISN | (SEQ | (SEQ ID |
| | | | Y (SEQ | 1002) | LEQEDIATY | ID NO: | NO: |
| | | | ID NO: | | FC (SEQ ID | 1272) | 959) |
| | | | 1088) | | NO: 1151) | | |
| BP003T3P2-29 | DIQMTQTTSSLSASL | QDISNY | LNWYQ | HTS | RLQSGVPS | QQSNS | FGGGT |
| | GDRVTISCRAS (SEQ | (SEQ ID | QKPDG | (SEQ ID | RFTGSGSG | LPPT | KLEIK |
| | ID NO: 760) | NO: 27) | TVKLLI | NO: | TDYSLTISN | (SEQ | (SEQ ID |
| | | | Y (SEQ | 1002) | LEQEDIATY | ID NO: | NO: |
| | | | ID NO: | | FC (SEQ ID | 1271) | 959) |
| | | | 1088) | | NO: 1355) | | |
| BP003T3P2-3 | DIQMTQSPASLSAS | ENIYSY | LEWYQ | NAK | TLVEGVPS | QHHY | FGGGT |
| | VGETVTITCRAS | (SEQ ID | QKQGK | (SEQ ID | RFSGSGSGT | DTPYT | KLEIK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 30) | 959) |
| | | | NO: | | NO: 1408) | | |
| | | | 1072) | | | | |
| BP003T3P2-30 | DIQMTQTTSSLSASL | QDISNY | LNWYQ | HTS | RLQSGVPS | QQSNS | FGGGT |
| | GDRVTISCRAS (SEQ | (SEQ ID | QKPDG | (SEQ ID | RFTGSGSG | LPPT | KLEIK |
| | ID NO: 760) | NO: 27) | TVKLLI | NO: | TDYSLTISN | (SEQ | (SEQ ID |
| | | | Y (SEQ | 1002) | LEQEDIATY | ID NO: | NO: |
| | | | ID NO: | | FC (SEQ ID | 1271) | 959) |
| | | | 1088) | | NO: 1355) | | |
| BP003T3P2-31 | DIQMTQTTSSLSASL | QDISNY | LNWYQ | HTS | RLQSGVPS | QQSNS | FGGGT |
| | GDRVTISCRAS (SEQ | (SEQ ID | QKPDG | (SEQ ID | RFTGSGSG | LPPT | KLEIK |
| | ID NO: 760) | NO: 27) | TVKLLI | NO: | TDYSLTISN | (SEQ | (SEQ ID |
| | | | Y (SEQ | 1002) | LEQEDIATY | ID NO: | NO: |
| | | | ID NO: | | FC (SEQ ID | 1271) | 959) |
| | | | 1088) | | NO: 1355) | | |
| BP003T3P2-32 | DIQMTQSTSSLSASL | QDISNY | LNWYQ | YTS | RLHSGVPS | QQGN | FGGGT |
| | GDRVTISCRAS (SEQ | (SEQ ID | QKPDG | (SEQ ID | RFSGSGSGT | TLPPT | KLEIK |
| | ID NO: 738) | NO: 27) | TVKLLI | NO: | DYSLTISNL | (SEQ | (SEQ ID |
| | | | Y (SEQ | 1495) | EQEDIATYF | | |
| | | | ID NO: | | C (SEQ ID | ID NO: | NO: |
| | | | 1088) | | NO: 1353) | 1253) | 959) |
| BP003T3P2-33 | DIQMTQTTSSLSASL | QDISNY | LNWYQ | HTS | RLQSGVPS | QQSNS | FGGGT |
| | GDRVTISCRAS (SEQ | (SEQ ID | QKPDG | (SEQ ID | RFTGSGSG | LPPT | KLEIK |
| | ID NO: 760) | NO: 27) | TVKLLI | NO: | TDYSLTISN | (SEQ | (SEQ ID |
| | | | Y (SEQ | 1002) | LEQEDIATY | ID NO: | NO: |
| | | | ID NO: | | FC (SEQ ID | 1271) | 959) |
| | | | 1088) | | NO: 1355) | | |
| BP003T3P2-34 | DIQMTQTTSSLSASL | QDISNY | LNWYQ | HTS | RLQSGVPS | QQSNS | FGGGT |
| | GDRVTISCRAS (SEQ | (SEQ ID | QKPDG | (SEQ ID | RFTGSGSG | LPPT | KLEIK |
| | ID NO: 760) | NO: 27) | TVKLLI | NO: | TDYSLTISN | (SEQ | (SEQ ID |
| | | | Y (SEQ | 1002) | LEQEDIATY | ID NO: | NO: |
| | | | ID NO: | | FC (SEQ ID | 1271) | 959) |
| | | | 1088) | | NO: 1355) | | |
| BP003T3P2-35 | DIQMTQTTSSLSASL | QDISNY | LNWYQ | HTS | NLQSGVPS | QQSNT | FGGGT |
| | GDRVTISCRAS (SEQ | (SEQ ID | QKPDG | (SEQ ID | RFTGSGSG | LPPT | KLEIK |
| | ID NO: 760) | NO: 27) | TVKLLI | NO: | TDYSLTISN | (SEQ | (SEQ ID |
| | | | Y (SEQ | 1002) | LEQEDIATY | ID NO: | NO: |
| | | | ID NO: | | FC (SEQ ID | 1272) | 959) |
| | | | 1088) | | NO: 1151) | | |
| BP003T3P2-36 | DIQMTQSTSSLSASL | QDISNY | LNWYQ | HTS | RLHSGVPS | QQSNT | FGGGT |
| | GDRVTITCRAS (SEQ | (SEQ ID | QKPDG | (SEQ ID | RFSGSESGT | LPPT | KLEIK |
| | ID NO: 740) | NO: 27) | TVKLLI | NO: | DYSLTISNL | (SEQ | (SEQ ID |
| | | | Y (SEQ | 1002) | EQEDIATYF | ID NO: | NO: |
| | | | ID NO: | | C (SEQ ID | 1272) | 959) |
| | | | 1088) | | NO: 1350) | | |
| BP003T3P2-37 | DVVMTQTTSSLSAS | QDISNY | LNWYQ | HTS | RLHSGVPS | QQGN | FGGGT |
| | LGDRVTITCRAS | (SEQ ID | QKPDG | (SEQ ID | RFSGSESGT | TLPPT | KLEIK |
| | (SEQ ID NO: 915) | NO: 27) | TVKLLI | NO: | DYSLTISNL | (SEQ | (SEQ ID |
| | | | Y (SEQ | 1002) | EQEDIATYF | ID NO: | NO: |
| | | | ID NO: | | C (SEQ ID | 1253) | 959) |
| | | | 1088) | | NO: 1350) | | |
| BP003T3P2-38 | DIQMTQSTSSLSASL | QDISNY | LNWYQ | YTS | RLHSGVPS | QQGN | FGGGT |
| | GDRVTISCRAS (SEQ | (SEQ ID | QKPDG | (SEQ ID | RFSGSGSGT | TLPPT | KLEIK |
| | ID NO: 738) | NO: 27) | TVKLLI | NO: | DYSLTISNL | (SEQ | (SEQ ID |
| | | | Y (SEQ | 1495) | EQEDIATYF | ID NO: | NO: |
| | | | ID NO: | | C (SEQ ID | 1253) | 959) |
| | | | 1088) | | NO: 1353) | | |
| BP003T3P2-39 | DIVMTQSTSSLSASL | QDISNY | LNWYQ | HTS | RLHSGVPS | QQGN | FGGGT |
| | GDRVTISCRAS (SEQ | (SEQ ID | QKPDG | (SEQ ID | RFSGSESGT | TLPPT | KLEIK |
| | ID NO: 828) | NO: 27) | TVKLLI | NO: | DYSLTISNL | (SEQ | (SEQ ID |
| | | | Y (SEQ | 1002) | EQEDIATYF | ID NO: | NO: |
| | | | ID NO: | | C (SEQ ID | 1253) | 959) |
| | | | 1088) | | NO: 1350) | | |
| BP003T3P2-4 | DIQMTQSPASLSAS | ENIYSY | LEWYQ | NAK | TLVEGVPS | QHHY | FGGGT |
| | VGETVTITCRAS | (SEQ ID | QKQGK | (SEQ ID | RFSGSGSGT | DTPYT | KLEIK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 30) | 959) |
| | | | NO: | | NO: 1408) | | |
| | | | 1072) | | | | |
| BP003T3P2-40 | DIVMTQSQKFMSTS | QNVGT | VAWY | SAS | YRYSGVPD | QQYN | FGGGT |
| | VGDRVSVTCKAS | N (SEQ | QQKPG | (SEQ ID | RFTGSGSG | SYPYT | KLEIK |
| | (SEQ ID NO: 821) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: 1483) | | |
| | | | 1425) | | | | |
| BP003T3P2-41 | DIVITQSQKFMSTSV | QNVGT | VAWY | SAS | YRYSGVPD | QQYN | FGGGT |
| | GDRVSVTCKAS | N (SEQ | QQKPG | (SEQ ID | RFTGSGSG | SYPYT | KLEIK |
| | (SEQ ID NO: 766) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: 1483) | | |
| | | | 1425) | | | | |
| BP003T3P2-42 | DIQMTQSQKFMSTS | QNVGT | VAWY | SAS | YRYSGVPD | QQYN | FGGGT |
| | VGDRVSVTCKAS | N (SEQ | QQKPG | (SEQ ID | RFTGSGSG | SYPYT | KLEIK |
| | (SEQ ID NO: 734) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: 1483) | | |
| | | | 1425) | | | | |
| BP003T3P2-43 | DIVLTQSQKFMSTS | QNVGT | VAWY | SAS | YRYSGVPD | QQYN | FGGGT |
| | VGDRVSVTCKAS | N (SEQ | QQKPG | (SEQ ID | RITGRGSGT | TYPYT | KLEIK |
| | (SEQ ID NO: 773) | ID NO: | QSPKA | NO: 13) | DFTLTISNV | (SEQ | (SEQ ID |
| | | 12) | LIY | | QSEDLADY | ID NO: | NO: |
| | | | (SEQ ID | | FC (SEQ ID | 1287) | 959) |
| | | | NO: | | NO: 1488) | | |
| | | | 1425) | | | | |
| BP003T3P2-44 | DIQMTQSQKFMSTS | QNVGT | VAWY | SAS | YRYSGVPD | QQYN | FGGGT |
| | AGDRVSVTCKAS | N (SEQ | QQKPG | (SEQ ID | RFTGSGSG | SYPYT | KLEIK |
| | (SEQ ID NO: 730) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1483) | | |
| BP003T3P2-45 | DIVLTQSQKFMSTS | QNVGT | VAWY | SAS | YRYSGVPD | QQYN | FGGGT |
| | VGDRVSVTCKAS | N (SEQ | QQKPG | (SEQ ID | RFTGSGSG | TYPYT | KLEMK |
| | (SEQ ID NO: 773) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 1287) | 961) |
| | | | NO: | | ID NO: | | |
| | | | 1425) | | 1483) | | |
| BP003T3P2-46 | RCPDDPSPSSLSASL | QDIGLN | LNWLQ | ATS | SLDSGVPK | LQYAS | FGAGT |
| | GERVSLTCRAS | (SEQ ID | QEPDG | (SEQ ID | RFSGSRSGS | SPFT | KLEIK |
| | (SEQ ID NO: 1347) | NO: | TIKRLI | NO: | DYSLTISSL | (SEQ | (SEQ ID |
| | | 1198) | Y (SEQ | 618) | ESEDFVDY | ID NO: | NO: |
| | | | ID NO: | | YC (SEQ ID | 1097) | 958) |
| | | | 1085) | | NO: 1366) | | |
| BP003T3P2-47 | DIVLTQSQKFMSTS | QNVGT | VAWY | SAS | YRYSGVPD | QQYN | FGGGT |
| | VGDRVSVTCKAS | N (SEQ | QQKPG | (SEQ ID | RITGRGSGT | TYPYT | KLEIK |
| | (SEQ ID NO: 773) | ID NO: | QSPKA | NO: 13) | DFTLTISNV | (SEQ | (SEQ ID |
| | | 12) | LIY | | QSEDLADY | ID NO: | NO: |
| | | | (SEQ ID | | FC (SEQ ID | 1287) | 959) |
| | | | NO: | | NO: 1488) | | |
| | | | 1425) | | | | |
| BP003T3P2-48 | DIQMTQSQKFMSTS | QNVGT | VAWY | SAS | YRYSGVPD | QQYN | FGGGT |
| | VGDRVSVTCKAS | N (SEQ | QQKPG | (SEQ ID | RFTGSGSG | SYPYT | KLEIK |
| | (SEQ ID NO: 734) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1483) | | |
| BP003T3P2-49 | DTTVTQSQKFMSTS | QNVGT | VAWY | SAS | YRYSGVPD | QQYN | FGGGT |
| | VGDRVSVTCKAS | N (SEQ | QQKPG | (SEQ ID | RFTGSGSG | SYPYT | KLEMK |
| | (SEQ ID NO: 858) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 961) |
| | | | NO: | | ID NO: | | |
| | | | 1425) | | 1483) | | |
| BP003T3P2-5 | DIQMTQSPASLSAS | ENIYSY | LEWYQ | NAK | TLAEGVPS | QHHY | FGGGT |
| | VGETVTITCRAS | (SEQ ID | QKQGK | (SEQ ID | RFSGSGSGT | DTPYT | KLEIK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 30) | 959) |
| | | | NO: | | NO: 1398) | | |
| | | | 1072) | | | | |
| BP003T3P2-50 | DIVMTQSQKFMSTS | QNVGT | VAWY | SAS | YRYSGVPD | QQYN | FGGGT |
| | VGDRVSVTCKAS | N (SEQ | QQKPG | (SEQ ID | RFTGSGSG | SYPYT | KLEIK |
| | (SEQ ID NO: 821) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1483) | | |
| BP003T3P2-51 | MLVMTQTPLSLPVS | QSLVH | LHWYL | KVS | NRFSGVPD | SQSTH | FGGGT |
| | LGDQASISCRSS | SNGNT | QKPGQ | (SEQ ID | RFSVSGSGT | VPPT | KLEIK |
| | (SEQ ID NO: 1132) | Y (SEQ | SPKLLI | NO: | DFTLKISRV | (SEQ | (SEQ ID |
| | | ID NO: | Y (SEQ | 1062) | EAEDLGVY | ID NO: | NO: |
| | | 1306) | ID NO: | | FC (SEQ ID | 1371) | 959) |
| | | | 1076) | | NO: 1159) | | |
| BP003T3P2-52 | DTTVTQSQKFMSTS | QNVGT | VAWY | SAS | YRYSGVPD | QQYN | FGGGT |
| | VGDRVSVTCKAS | N (SEQ | QQKPG | (SEQ ID | RFTGSGSG | SYPYT | KLEMK |
| | (SEQ ID NO: 858) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 961) |
| | | | NO: | | ID NO: | | |
| | | | 1425) | | 1483) | | |
| BP003T3P2-53 | DVQMTQSQKFMST | QNVGT | VAWY | SAS | YRYSGVPD | QQYN | FGGGT |
| | SVGDRVSVTCKAS | N (SEQ | QQKPG | (SEQ ID | RFTGSGSG | SYPYT | KLEIK |
| | (SEQ ID NO: 873) | ID NO: | QSPKA | NO: 13) | TDFTLTISN | (SEQ | (SEQ ID |
| | | 12) | LIY | | VQSEDLAE | ID NO: | NO: |
| | | | (SEQ | | YFC (SEQ | 15) | 959) |
| | | | ID | | ID NO: | | |
| | | | NO: | | 1483) | | |
| | | | 1425) | | | | |
| BP003T3P2-54 | DIQMTQSQKFMSTS | QNVGT | VAWY | SAS | YRYSGVPD | QQYN | FGGGT |
| | VGDRVSVTCKAS | N (SEQ | QQKPG | (SEQ ID | RFTGSGSG | SYPYT | KLEIK |
| | (SEQ ID NO: 734) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1425) | | 1483) | | |
| BP003T3P2-55 | DILLTQSQKFMSTSV | QNVGT | VAWY | SAS | YRYSGVPD | QQYN | FGGGT |
| | GDRVSVTCKAS | N (SEQ | QQKPG | (SEQ ID | RFTGSGSG | SYPYT | KLEMK |
| | (SEQ ID NO: 700) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 961) |
| | | | NO: | | ID NO: | | |
| | | | 1425) | | 1483) | | |
| BP003T3P2-56 | DIVLTQSQKFMSTS | QNVGT | VAWY | SAS | YRYSGVPD | QQYN | FGGGT |
| | VGDRVSVTCKAS | N (SEQ | QQEPG | (SEQ ID | RFTGSGSG | SYPYT | KLEIK |
| | (SEQ ID NO: 773) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 959) |
| | | | NO: | | ID NO: | | |
| | | | 1423) | | 1483) | | |
| BP003T3P2-57 | DILLTQSQKFMSTSV | QNVGT | VAWY | SAS | YRYSGVPD | QQYN | FGGGT |
| | GDRVSVTCKAS | N (SEQ | QQKPG | (SEQ ID | RFTGSGSG | SYPYT | KLEMK |
| | (SEQ ID NO: 700) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 961) |
| | | | NO: | | ID NO: | | |
| | | | 1425) | | 1483) | | |
| BP003T3P2-58 | DVVMTQSQKFMST | QNVGT | VAWY | SAS | YRYSGVPD | QQYN | FGGGT |
| | SVGDRVSVTCKAS | N (SEQ | QQKPG | (SEQ ID | RFTGSGSG | SYPYT | KLEMK |
| | (SEQ ID NO: 894) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 15) | 961) |
| | | | NO: | | ID NO: | | |
| | | | 1425) | | 1483) | | |
| BP003T3P2-59 | RHCESQSHKFMSTSI | QDVRT | VAWY | STS | YRYSGVPD | QQYS | GAGTK |
| | GDRVSITCKAS | A (SEQ | QQKPG | (SEQ ID | RFTGSGSG | NYLTF | LEIK |
| | (SEQ ID NO: | ID NO: | QSPKL | NO: | TDFTLTINS | (SEQ | (SEQ ID |
| | 1349) | 1208) | LIY | 1386) | VQSEDLAD | ID NO: | NO: |
| | | | (SEQ ID | | YFC (SEQ | 1290) | 974) |
| | | | NO: | | ID NO: | | |
| | | | 1427) | | 1481) | | |
| BP003T3P2-6 | DIQMTQSPASLSAS | ENIYSY | LEWYQ | NAK | TLAEGVPS | QHHY | FGGGT |
| | VGETVTITCRAS | (SEQ ID | QKQGK | (SEQ ID | RFSGSGSGT | DTPYT | KLEIK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 30) | 959) |
| | | | NO: | | NO: 1398) | | |
| | | | 1072) | | | | |
| BP003T3P2-60 | DIVLTQSQKFMSTS | QNVGT | VAWY | SAS | YRYSGVPD | QQYN | FGGGT |
| | VGDRVSVTCKAS | N (SEQ | QQKPG | (SEQ ID | RITGRGSGT | TYPYT | KLEIK |
| | (SEQ ID NO: 773) | ID NO: | QSPKA | NO: 13) | DFTLTISNV | (SEQ | (SEQ ID |
| | | 12) | LIY | | QSEDLADY | ID NO: | NO: |
| | | | (SEQ ID | | FC (SEQ ID | 1287) | 959) |
| | | | NO: | | NO: 1488) | | |
| | | | 1425) | | | | |
| BP003T3P2-61 | DVHMNQSQKFMST | QNVGT | VXWY | SAS | YRYNGVPD | XQYN | FGGGT |
| | SVGNRVSVTCKGS | N (SEQ | QQKPG | (SEQ ID | RFTGSGSG | SYPYT | XLEIK |
| | (SEQ ID NO: 865) | ID NO: | QSPKA | NO: 13) | TDFTLTISN | (SEQ | (SEQ ID |
| | | 12) | LIY | | VQSEDLAE | ID NO: | NO: |
| | | | (SEQ ID | | YFC (SEQ | 1475) | 963) |
| | | | NO: | | ID NO: | | |
| | | | 1449) | | 1478) | | |
| BP003T3P2-62 | DIVMTQTPKFLLVS | QSVNN | WWY | YAS | NRYTGVPD | QQAY | FGGGT |
| | AGDRVTITCKAS | D (SEQ | QQKPG | (SEQ ID | RFTGSGYG | WSPY | KLEIK |
| | (SEQ ID NO: 830) | ID NO: | QSPKL | NO: | TDFTFTIST | T (SEQ | (SEQ ID |
| | | 1309) | LIY | 1476) | VQAEDLAV | ID NO: | NO: |
| | | | (SEQ ID | | YFC (SEQ | 1248) | 959) |
| | | | NO: | | ID NO: | | |
| | | | 1447) | | 1164) | | |
| BP003T3P2-63 | DIVMTQSPPTLSLSP | QDVNT | VAWY | WAS | TRHTGVPS | QQHY | FGGGT |
| | GERVTLSCKAS | A (SEQ | QQKPG | (SEQ ID | RFSGSGSGT | SSPWT | KLEIK |
| | (SEQ ID NO: 816) | ID NO: | QAPRL | NO: | DFTLTISSL | (SEQ | (SEQ ID |
| | | 1206) | LIY | 1450) | QPEDFATY | ID NO: | NO: |
| | | | (SEQ ID | | YC (SEQ ID | 1258) | 959) |
| | | | NO: | | NO: 1414) | | |
| | | | 1424) | | | | |
| BP003T3P2-64 | EIVMTQSPPTLSLSP | QDVNT | VAWY | WAS | TRHTGVPS | QQHY | FGGGT |
| | GERVTLSCKAS | A (SEQ | QQKPG | (SEQ ID | RFSGSGSGT | SSPWT | KLEIK |
| | (SEQ ID NO: 926) | ID NO: | QAPRL | NO: | DFTLTISSL | (SEQ | (SEQ ID |
| | | 1206) | LIY | 1450) | QPEDFATY | ID NO: | NO: |
| | | | (SEQ ID | | YC (SEQ ID | 1258) | 959) |
| | | | NO: | | NO: 1414) | | |
| | | | 1424) | | | | |
| BP003T3P2-65 | DIVMTQSPPTLSLSP | QDVNT | VAWY | WAS | TRHTGVPS | QQHY | FGGGT |
| | GERVTLSCKAS | A (SEQ | QQKPG | (SEQ ID | GFSGSGSG | SSPWT | KLEIK |
| | (SEQ ID NO: 816) | ID NO: | QAPRL | NO: | TDFTLTISS | (SEQ | (SEQ ID |
| | | 1206) | LIY | 1450) | LQPEDFAT | ID NO: | NO: |
| | | | (SEQ ID | | YYC (SEQ | 1258) | 959) |
| | | | NO: | | ID NO: | | |
| | | | 1424) | | 1413) | | |
| BP003T3P2-66 | RCQMNQSPSSLSAS | QEISGY | LSWLQ | AAS | TLDSGVPK | LQYAS | FGGGT |
| | LGERVSLTCRAS | (SEQ ID | QKPDG | (SEQ ID | RFSGSRSGS | YPYT | KLEMK |
| | (SEQ ID NO: 1348) | NO: | TIKRLT | NO: | DYSLTISSL | (SEQ | (SEQ ID |
| | | 1210) | Y (SEQ | 599) | ESEDFADY | ID NO: | NO: |
| | | | ID NO: | | YC (SEQ ID | 1103) | 961) |
| | | | 1112) | | NO: 1402) | | |
| BP003T3P2-67 | DVVLTQTPLSLPVSL | QSIVHS | LEWYL | KVS | NRFSGVPD | FQGSH | FGGGT |
| | GDQASISCRSS (SEQ | NGNTY | QKPGQ | (SEQ ID | RFSGSGSGT | VPPT | KLEMK |
| | ID NO: 874) | (SEQ ID | SPKLLI | NO: | DFTLKISRV | (SEQ | (SEQ ID |
| | | NO: | Y (SEQ | 1062) | EAEDLGVY | ID NO: | NO: |
| | | 1299) | ID NO: | | YC (SEQ ID | 966) | 961) |
| | | | 1071) | | NO: 1158) | | |
| BP003T3P2-68 | DVVVTQTPLSLPVS | QSIVHS | LEWYL | KVS | NRFSGVPD | FQGSH | FGGGT |
| | LGDQASISCRSS | NGNTY | QKPGQ | (SEQ ID | RFSGSGSGT | VPRT | KLEIK |
| | (SEQ ID NO: 918) | (SEQ ID | SPKLLI | NO: | DFTLKISRV | (SEQ | (SEQ ID |
| | | NO: | Y (SEQ | 1062) | EAEDLGVY | ID NO: | NO: |
| | | 1299) | ID NO: | | YC (SEQ ID | 967) | 959) |
| | | | 1071) | | NO: 1158) | | |
| BP003T3P2-69 | DVVMTQTPLSLPVS | QSIVHN | LEWYL | KVS | NRFSGVPD | FQGSY | FGGGT |
| | LGDQASISCRSN | NGNTY | QKPGQ | (SEQ ID | RFSGSGSGT | VPRT | KLEIK |
| | (SEQ ID NO: 899) | (SEQ ID | SPKLLI | NO: | DFTLKISRV | (SEQ | (SEQ ID |
| | | NO: | Y (SEQ | 1062) | EAEDLGVY | ID NO: | NO: |
| | | 1298) | ID NO: | | YC (SEQ ID | 971) | 959) |
| | | | 1071) | | NO: 1158) | | |
| BP003T3P2-7 | DIQMTQSPASLSAS | ENIYSY | LEWYQ | NAK | TLAEGVPS | QHHY | FGGGT |
| | VGETVTITCRAS | (SEQ ID | QKQGK | (SEQ ID | RFSGSGSGT | DTPYT | KLEIK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 30) | 959) |
| | | | NO: | | NO: 1398) | | |
| | | | 1072) | | | | |
| BP003T3P2-70 | DVVMTQTPLSLPVS | QSIVHS | LEWYL | KVS | NRFSGVPD | FQGSH | FGGGT |
| | LGDQASISCRSS | NGNTY | QKPGQ | (SEQ ID | RFSGSGSGT | VPPT | KLEIK |
| | (SEQ ID NO: 900) | (SEQ ID | SPKLLI | NO: | DFTLKISRV | (SEQ | (SEQ ID |
| | | NO: | Y (SEQ | 1062) | EAEDLGVY | ID NO: | NO: |
| | | 1299) | ID NO: | | YC (SEQ ID | 966) | 959) |
| | | | 1071) | | NO: 1158) | | |
| BP003T3P2-71 | DVVLTQTPLSLPVSL | QSIVHS | LEWYL | KVS | NRFSGVPD | FQGSH | FGGGT |
| | GDQASISCRSS (SEQ | NGNTY | QKPGQ | (SEQ ID | RFSGSGSGT | VPPT | KLEIK |
| | ID NO: 874) | (SEQ ID | SPKLLI | NO: | DFTLKISRV | (SEQ | (SEQ ID |
| | | NO: | Y (SEQ | 1062) | EAEDLGVY | ID NO: | NO: |
| | | 1299) | ID NO: | | YC (SEQ ID | 966) | 959) |
| | | | 1071) | | NO: 1158) | | |
| BP003T3P2-72 | DVVMTQTPLSLPVS | QSIVHS | LEWYL | KVS | NRFSGVPD | FQGSH | GGGTK |
| | LGDQASISCRSS | NGNTY | QKPGQ | (SEQ ID | RFSGSGSGT | VPTF | KLEIK |
| | (SEQ ID NO: 900) | (SEQ ID | SPKLLI | NO: | DFTLKISRV | (SEQ | (SEQ ID |
| | | NO: | Y (SEQ | 1062) | EAEDLGVY | ID NO: | NO: |
| | | 1299) | ID NO: | | YC (SEQ ID | 968) | 978) |
| | | | 1071) | | NO: 1158) | | |
| BP003T3P2-73 | DVVVTQTPLSLPVS | QSIVHS | LEWYL | KVS | NRFSGVPD | FQGSH | FGGGT |
| | LGDQASISCRSS | NGNTY | QKPGQ | (SEQ ID | RFSGSGSGT | VPPT | KLEIK |
| | (SEQ ID NO: 918) | (SEQ ID | SPKLLI | NO: | DFTLKISRV | (SEQ | (SEQ ID |
| | | NO: | Y (SEQ | 1062) | EAEDLGVY | ID NO: | NO: |
| | | 1299) | ID NO: | | YC (SEQ ID | 966) | 959) |
| | | | 1071) | | NO: 1158) | | |
| BP003T3P2-74 | DVVLTQTPLSLPVSL | QSIVHS | LEWYL | KVS | NRFSGVPD | FQGSH | FGGGT |
| | GDQASISCRSS (SEQ | NGNTY | QKPGQ | (SEQ ID | RFSGSGSGT | VPPT | KLEIK |
| | ID NO: 874) | (SEQ ID | SPKLLI | NO: | DFTLKISRV | (SEQ | (SEQ ID |
| | | NO: | Y | 1062) | EAEDLGVY | ID NO: | NO: |
| | | 1299) | (SEQ | | YC | 966) | 959) |
| | | | ID NO: | | (SEQ ID | | |
| | | | 1071) | | NO: 1158) | | |
| BP003T3P2-75 | DIQMNQSHKFMSTS | QDVST | VAWY | SAS | YRYTGVPD | QQHY | FGGGT |
| | VGDRVSITCKAS | A (SEQ | QQKPG | (SEQ ID | RFTGSGSG | STPYT | KLEIK |
| | (SEQ ID NO: 713) | ID NO: | QSPKL | NO: 13) | TDFTFTISS | (SEQ | (SEQ ID |
| | | 1209) | LIY | | VQAEDLAV | ID NO: | NO: |
| | | | (SEQ ID | | YYC (SEQ | 1265) | 959) |
| | | | NO: | | ID NO: | | |
| | | | 1427) | | 1491) | | |
| BP003T3P2-8 | DIQMTQSPASLSAS | ENIYSY | LEWYQ | NAK | TLAEGVPS | QHHY | FGGGT |
| | VGETVTITCRAS | (SEQ ID | QKQGK | (SEQ ID | RFSGSGSGT | DTPYT | KLEIK |
| | (SEQ ID NO: 720) | NO: 930) | SPQLL | NO: 28) | QFSLKINSL | (SEQ | (SEQ ID |
| | | | VY | | QPEDFGSY | ID NO: | NO: |
| | | | (SEQ ID | | YC (SEQ ID | 30) | 959) |
| | | | NO: | | NO: 1398) | | |
| | | | 1072) | | | | |
| BP003T3P2-9 | DIVMTQSPASLSAS | DNIYN | LEWYQ | NAK | TLGEGVPS | QHHY | FGGGT |
| | VGETVTITCRAS | Y (SEQ | QKQGK | (SEQ ID | RFSGSGSGT | GSPW | KLEIK |
| | (SEQ ID NO: 807) | ID NO: | SPQLL | NO: 28) | QFYLKINSL | T (SEQ | (SEQ ID |
| | | 833) | VY | | QPEDFGSY | ID NO: | NO: |
| | | | (SEQ ID | | YC (SEQ ID | 1215) | 959) |
| | | | NO: | | NO: 1404) | | |
| | | | 1072) | | | | |

TABLE 5A
Exemplary Clones-Heavy Chain Sequences

| ID | HFR1 | CDRH1 | HFR2 | CDRH2 | HFR3 | CDRH3 | HFR4 |
|---|---|---|---|---|---|---|---|
| AH04507 | QVQLQQSGAELVRPG | GYTFTNYW | IGWVK | IHPGGG | DYNEKFT | VSRNFA | WGQGT |
| | TSVKMSCKAA (SEQ | (SEQ ID NO: | QRPGH | YI (SEQ | GKATLTA | K (SEQ ID | LVTVSA |
| | ID NO: 1326) | 4) | GLEWI | ID NO: | DTSSSTA | NO: 1444) | (SEQ ID |
| | | | GD | 1014) | YMQLSSL | | NO: 421) |
| | | | (SEQ ID | | TSEDSAIY | | |
| | | | NO: | | YC (SEQ | | |
| | | | 1010) | | ID NO: | | |
| | | | | | 923) | | |
| AH04522 | QIQLQQSGAELVRPGT | GYTFTNYW | IGWVK | IHPGGG | NYNEKFK | TSRNFAY | WGQGT |
| | SVKMSCKAA (SEQ | (SEQ ID NO: | QRPGH | YT (SEQ | GKATLTA | (SEQ ID | LVTVSA |
| | ID NO: 1228) | 4) | GLEWI | ID NO: | DTSSSTA | NO: 1419) | (SEQ ID |
| | | | GD | 1015) | YMQLSSL | | NO: 421) |
| | | | (SEQ ID | | TSEDSAIY | | |
| | | | NO: | | YC (SEQ | | |
| | | | 1010) | | ID NO: | | |
| | | | | | 1177) | | |
| AH04526 | QVQLQQSGAELVRPG | GYTFTNYW | IGWVK | IHPGGD | NYNEKFK | TSRNFAY | WGQGT |
| | TSVKMSCKAA (SEQ | (SEQ ID NO: | QRPGH | YS (SEQ | GKATLTA | (SEQ ID | PVTVSS |
| | ID NO: 1326) | 4) | GLEWI | ID NO: | DTSSSTA | NO: 1419) | (SEQ ID |
| | | | GD | 1012) | YMNLSSL | | NO: 398) |
| | | | (SEQ ID | | TSEDSAIY | | |
| | | | NO: | | YC (SEQ | | |
| | | | 1010) | | ID NO: | | |
| | | | | | 1174) | | |
| AH04527 | QVQLQQSGAELVRPG | GYTFTNYW | IGWVK | IHPGGG | DYNEKFT | VSRNFA | WGQGT |
| | TSVKMSCKAA (SEQ | (SEQ ID NO: | QRPGH | YI (SEQ | GKATLTA | K (SEQ ID | LVTVSA |
| | ID NO: 1326) | 4) | GLEWI | ID NO: | DTSSSTA | NO: 1444) | (SEQ ID |
| | | | GD | 1014) | YMQLSSL | | NO: 421) |
| | | | (SEQ ID | | TSEDSAIY | | |
| | | | NO: | | YC (SEQ | | |
| | | | 1010) | | ID NO: | | |
| | | | | | 923) | | |
| AH04734 | QVQLQQPAAELARPG | GYTFTTST | MHWV | INPRSG | EYNQKFK | ARHYYF | WGQGT |
| | ASVKMSCKAS (SEQ | (SEQ ID NO: | KQRPG | YT (SEQ | DKTTLTA | DY (SEQ | TVTVSS |
| | ID NO: 467) | 993) | QGLEW | ID NO: | DKSSSTA | ID NO: | (SEQ ID |
| | | | IGY | 1024) | YMQLSSL | 612) | NO: 422) |
| | | | (SEQ ID | | TSEDSAV | | |
| | | | NO: | | YYC (SEQ | | |
| | | | 190) | | ID NO: | | |
| | | | | | 519) | | |
| AH04750 | QVQLQQPGAEMAKP | GYTSTAYW | IHWVK | ITPSTGY | EYNQKFE | ARGGYF | WGQGT |
| | GASVKMSCKAS (SEQ | (SEQ ID NO: | QRPGQ | T (SEQ | DKATLTA | DY (SEQ | TVTVSS |
| | ID NO: 1320) | 994) | GLEWI | ID NO: | DKSSNTA | ID NO: | (SEQ ID |
| | | | GY | 1028) | YMQLNSL | 606) | NO: 422) |
| | | | (SEQ ID | | TSEDSAV | | |
| | | | NO: | | YYC (SEQ | | |
| | | | 196) | | ID NO: | | |
| | | | | | 953) | | |
| AH05214 | QIQLQQPGAELVKPG | GYTFTSYW | MHWV | INPSNG | NYNEKFK | ARQLAA | WGQGT |
| | ASVKLPCKAS (SEQ | (SEQ ID NO: | KQRPG | RT (SEQ | SKATLTV | Y (SEQ ID | TVTVSS |
| | ID NO: 1219) | 157) | QGLEW | ID NO: | DKSSSTA | NO: 614) | (SEQ ID |
| | | | IGE | 510) | YMQLSSL | | NO: 422) |
| | | | (SEQ ID | | TSEDSAV | | |
| | | | NO: | | YYC (SEQ | | |
| | | | 209) | | ID NO: | | |
| | | | | | 289) | | |
| AH05247 | QVQLQQSGADLMKP | GYTFSNYW | IEWIKQ | ILPGSGF | NYNENFK | ARGGTS | WGQGT |
| | GASVKMSCRAS (SEQ | (SEQ ID NO: | RPGHG | T (SEQ | GKATFTA | WHFDS | TLTVSS |
| | ID NO: 1321) | 986) | LEWIG | ID NO: | DTSSNTT | (SEQ ID | (SEQ ID |
| | | | E (SEQ | 1021) | YMLLSSL | NO: 604) | NO: 425) |
| | | | ID NO: | | SSEDSAV | | |
| | | | 1005) | | YYC (SEQ | | |
| | | | | | ID NO: | | |
| | | | | | 1187) | | |
| AH05249 | QVQLQQSGADLMKP | GYTFSNYW | IEWIKQ | ILPGSGF | NYNENFK | ARGGTS | WGQGT |
| | GASVKMSCRAS (SEQ | (SEQ ID NO: | RPGHG | T (SEQ | GKATFTA | WHFDS | TLTVSS |
| | ID NO: 1321) | 986) | LEWIG | ID NO: | DTSSNTT | (SEQ ID | (SEQ ID |
| | | | E (SEQ | 1021) | YMLLSSL | NO: 604) | NO: 425) |
| | | | ID NO: | | SSEDSAV | | |
| | | | 1005) | | YYC (SEQ | | |
| | | | | | ID NO: | | |
| | | | | | 1187) | | |
| AH05251 | EVRLQQSGAELVRSG | GFNIKDYY | IHWVK | IDPDNG | EYAPKFQ | TVFWYG | WGQGT |
| | ASVKLSCTAS (SEQ | (SEQ ID NO: | QRPEQ | ET (SEQ | GKATMT | NNYAGF | LVTVSS |
| | ID NO: 943) | 977) | GLEWI | ID NO: | ADTSSNT | AY (SEQ | (SEQ ID |
| | | | GW | 1003) | AHLQLSS | ID NO: | NO: 413) |
| | | | (SEQ ID | | LTSEDTA | 1420) | |
| | | | NO: | | VYYC | | |
| | | | 1017) | | (SEQ ID | | |
| | | | | | NO: 946) | | |
| AH05256 | QVQLQQPGAELAKPG | GYTFTRYW | IHWVK | INPRTD | EYNQKFK | ARHGYF | WGQGT |
| | ASVKMSCKAS (SEQ | (SEQ ID NO: | QRPGQ | YT (SEQ | DKATLTA | DY (SEQ | TLTVSS |
| | ID NO: 114) | 992) | DLEWI | ID NO: | DKSSSTA | ID NO: | (SEQ ID |
| | | | GY | 1025) | YMQLSSL | 611) | NO: 425) |
| | | | (SEQ ID | | TSDDSAV | | |
| | | | NO: | | YYC (SEQ | | |
| | | | 1018) | | ID NO: | | |
| | | | | | 955) | | |
| AH05257 | QVQLQQSGAELVRPG | GYTFTDYA | MHWV | ISTYSG | IYNQKFK | ARGVTF | WGQGT |
| | VSLKISCKGS (SEQ | (SEQ ID NO: | RQSHA | DA (SEQ | GKATMT | DY (SEQ | TLTVSA |
| | ID NO: 1337) | 19) | KSLEW | ID NO: | VDKSSST | ID NO: | (SEQ ID |
| | | | IGV | 21) | AYLELAR | 23) | NO: |
| | | | (SEQ ID | | LTSEDSAI | | 1452) |
| | | | NO: | | YYC (SEQ | | |
| | | | 1128) | | ID NO: | | |
| | | | | | 1034) | | |
| AH05258 | QIQLQQSGAELVRPG | GYTFTDYA | MHWV | ISTYSG | SYNQKFK | ARGVTF | WGQGT |
| | VSVKISCKGS (SEQ | (SEQ ID NO: | KQSHA | DV (SEQ | GKATMT | DS (SEQ | TLTVSS |
| | ID NO: 1240) | 19) | KSLEW | ID NO: | VDKSSST | ID NO: | (SEQ ID |
| | | | IGV | 46) | AYMELA | 48) | NO: 425) |
| | | | (SEQ ID | | RLTSEDS | | |
| | | | NO: | | AIYYC | | |
| | | | 1126) | | (SEQ ID | | |
| | | | | | NO: 1389) | | |
| AH05259 | QVQLQQSGAELVRPG | GYTFTDYA | MHWV | ISTYSG | SYNQKFK | ARGVTF | WGQGT |
| | VSVKISCKGS (SEQ | (SEQ ID NO: | KQSHA | DV (SEQ | GKATMT | DS (SEQ | SLTVSS |
| | ID NO: 1338) | 19) | KSLEW | ID NO: | VDKSSST | ID NO: | (SEQ ID |
| | | | IGV | 46) | AYMELA | 48) | NO: 424) |
| | | | (SEQ ID | | RLTSEDS | | |
| | | | NO: | | AIYYC | | |
| | | | 1126) | | (SEQ ID | | |
| | | | | | NO: 1389) | | |
| AH05268 | QVQLQQSGAELVRPG | GYTFTDYA | MHWV | ISTYSG | LYNQKFK | ARGVTF | WGQGT |
| | VSLKISCKGS (SEQ | (SEQ ID NO: | RQSHA | DA (SEQ | GKATMT | DY (SEQ | TLTVSS |
| | ID NO: 1337) | 19) | KSLEW | ID NO: | VDKSSST | ID NO: | (SEQ ID |
| | | | IGV | 21) | AYLELAR | 23) | NO: 425) |
| | | | (SEQ ID | | LTSDDSAI | | |
| | | | NO: | | YYC (SEQ | | |
| | | | 1128) | | ID NO: | | |
| | | | | | 1119) | | |
| AH05271 | QVQLQQSGAELVRPG | GYTFTDYA | MHWV | ISTYSG | SYNQKFK | ARGVTF | WGQGT |
| | VSVKISCKGS (SEQ | (SEQ ID NO: | KQSHA | DV (SEQ | GKATMT | DS (SEQ | TLTVSS |
| | ID NO: 1338) | 19) | KSLEW | ID NO: | VDKSSST | ID NO: | (SEQ ID |
| | | | IGV | 46) | AYMELA | 48) | NO: 425) |
| | | | (SEQ ID | | RLTSEDS | | |
| | | | NO: | | ATYYC | | |
| | | | 1126) | | (SEQ ID | | |
| | | | | | NO: 1390) | | |
| AH05274 | QVQLQQSGAELVRPG | GYTFTNYW | LGWIK | IYPGGG | NYNEKFK | ARVTPAS | WGQGT |
| | TSVKISCKAS (SEQ | (SEQ ID NO: | QRPGH | YT (SEQ | GKATLTA | (SEQ ID | SLTVSS |
| | ID NO: 1325) | 4) | GLEWI | ID NO: 6) | DTSSSTA | NO: 8) | (SEQ ID |
| | | | GD | | YMQLSSL | | NO: 424) |
| | | | (SEQ ID | | TSEDSAV | | |
| | | | NO: | | YFC (SEQ | | |
| | | | 1074) | | ID NO: | | |
| | | | | | 1178) | | |
| AH05280 | EVRLQQSGADLMKPG | GYTFSNYW | TEWIK | ILPGSGF | NYNENFK | ARGGTS | WGQGT |
| | ASVKISCKTS (SEQ | (SEQ ID NO: | QRPGH | T (SEQ | GKATFTA | WHFDY | SLTVSS |
| | ID NO: 941) | 986) | GLEWI | ID NO: | DTSSNTT | (SEQ ID | (SEQ ID |
| | | | GE | 1021) | YMQLSSL | NO: 605) | NO: 424) |
| | | | (SEQ ID | | TSEDSAV | | |
| | | | NO: | | YYC (SEQ | | |
| | | | 1392) | | ID NO: | | |
| | | | | | 1188) | | |
| AHO5285 | DVQLQESGAELAKPG | GYTFTRFW | MHWV | INPSTD | EYNQKFK | ARGTW | WGQGA |
| | ASVKMSCKAS (SEQ | (SEQ ID NO: | KQRPG | YT (SEQ | DKATLTA | DY (SEQ | TVTVSS |
| | ID NO: 866) | 991) | QGLEW | ID NO: | DKSSSTA | ID NO: | (SEQ ID |
| | | | IGY | 1027) | YMQLSSL | 607) | NO: |
| | | | (SEQ ID | | TSEDSAV | | 1451) |
| | | | NO: | | YYC (SEQ | | |
| | | | 190) | | ID NO: | | |
| | | | | | 265) | | |
| AH05286 | QVQLKQSGADLMKP | GYTFSNYW | IEWIKQ | ILPGSG | NYNENFK | ARGGTSF | WGQGT |
| | GASVKISCKAS (SEQ | (SEQ ID NO: | RPGHG | YT (SEQ | GKATFTA | VHFDY | TLTVSS |
| | ID NO: 1312) | 986) | LEWIG | ID NO: | DTSSNTT | (SEQ ID | (SEQ ID |
| | | | E (SEQ | 1022) | YIQLSSLS | NO: 602) | NO: 425) |
| | | | ID NO: | | SEDSAVY | | |
| | | | 1005) | | YC (SEQ | | |
| | | | | | ID NO: | | |
| | | | | | 1186) | | |
| AH4501 | QVQLQQPGAELVRPG | GYTFTNYW | IGWVK | IHPGGD | NYNEKFK | TSRNFAY | WGQGT |
| | TSVKMSCKAA (SEQ | (SEQ ID NO: | QRPGH | YS (SEQ | GKATLTA | (SEQ ID | LVTVSA |
| | ID NO: 1315) | 4) | GLEWI | ID NO: | DTSSSTA | NO: 1419) | (SEQ ID |
| | | | GD | 1012) | YMNLSSL | | NO: 421) |
| | | | (SEQ ID | | TSEDSAIY | | |
| | | | NO: | | YC (SEQ | | |
| | | | 1010) | | ID NO: | | |
| | | | | | 1174) | | |
| AH4502 | QIQLQQSGAELVRPGT | GYTFTNYW | IGWVK | IHPGGG | NYNEKFK | TSRNFAY | WGQGT |
| | SVKMSCKAA (SEQ | (SEQ ID NO: | QRPGH | YT (SEQ | GKATLTA | (SEQ ID | LVTVSA |
| | ID NO: 1228) | 4) | GLEWI | ID NO: | DTSSSTA | NO: 1419) | (SEQ ID |
| | | | GD | 1015) | DMQLSSL | | NO: 421) |
| | | | (SEQ ID | | TSEDSAIY | | |
| | | | NO: | | YC (SEQ | | |
| | | | 1010) | | ID NO: | | |
| | | | | | 1173) | | |
| AH4503 | QVQLQQPGAELVRPG | GYTFSNYW | IGWVK | IHPGGG | NYNEKFT | VSRNFA | WGQGT |
| | TSVKMSCKAA (SEQ | (SEQ ID NO: | QRPGH | YI (SEQ | GKATLTA | N (SEQ ID | LVTVSA |
| | ID NO: 1315) | 986) | GLEWI | ID NO: | DTSSSTA | NO: 1445) | (SEQ ID |
| | | | GD | 1014) | YMQLSSL | | NO: 421) |
| | | | (SEQ ID | | TSEDSAIY | | |
| | | | NO: | | YC (SEQ | | |
| | | | 1010) | | ID NO: | | |
| | | | | | 1184) | | |
| AH4505 | DVQLQQSGAELVRPG | GYTFTNYW | IGWVK | IHPGGG | NYNEKFK | TSRNFAY | WGQGT |
| | TSVKMSCKAA (SEQ | (SEQ ID NO: | QRPGH | YT (SEQ | GKATLTA | (SEQ ID | LVTVSA |
| | ID NO: 867) | 4) | GLEWI | ID NO: | DTSSSTA | NO: 1419) | (SEQ ID |
| | | | GD | 1015) | YMQLSSL | | NO: 421) |
| | | | (SEQ ID | | TSEGSAIY | | |
| | | | NO: | | YC (SEQ | | |
| | | | 1010) | | ID NO: | | |
| | | | | | 1179) | | |
| AH4509 | QIQLQQSGAELVRPGT | GYTFTNYW | IGWVK | FYPGGD | NYNEKFK | TSRNFAY | WGQGT |
| | SVKMSCKAA (SEQ | (SEQ ID NO: | QRPGH | YI (SEQ | GKATLTA | (SEQ ID | PVTVSS |
| | ID NO: 1228) | 4) | GLEWI | ID NO: | DTSSSTA | NO: 1419) | (SEQ ID |
| | | | GD | 973) | YMQLSSL | | NO: 398) |
| | | | (SEQ ID | | TSEDSAIY | | |
| | | | NO: | | YC (SEQ | | |
| | | | 1010) | | ID NO: | | |
| | | | | | 1177) | | |
| AH4511 | QIQLQQSGAELVRPGT | GYTFSNYW | IGWVK | IHPGGG | NYNEKFT | VSRNFA | WGQGT |
| | SVKMSCKAA (SEQ | (SEQ ID NO: | QRPGH | YI (SEQ | GKATLTA | N (SEQ ID | LVTVSA |
| | ID NO: 1228) | 986) | GLEWI | ID NO: | DTSSSTA | NO: 1445) | (SEQ ID |
| | | | GD | 1014) | YMQLSSL | | NO: 421) |
| | | | (SEQ ID | | TSEDSAIY | | |
| | | | NO: | | YC (SEQ | | |
| | | | 1010) | | ID NO: | | |
| | | | | | 1184) | | |
| AH4518 | QVQLQQSGAELVRPG | GYTFTNYW | IGWVK | IHPGGD | NYNEKFK | TSRNFAY | WGQGT |
| | TSVKMSCKAA (SEQ | (SEQ ID NO: | QRPGH | YS (SEQ | GKATLTA | (SEQ ID | LVTVSA |
| | ID NO: 1326) | 4) | DLEWI | ID NO: | DTSSSTA | NO: 1419) | (SEQ ID |
| | | | GD | 1012) | YMNLSSL | | NO: 421) |
| | | | (SEQ ID | | TSEDSAIY | | |
| | | | NO: | | YC (SEQ | | |
| | | | 1009) | | ID NO: | | |
| | | | | | 1174) | | |
| AH4523 | QIQLQQSGAELVRPGT | GYTFTNYW | IGWVK | IHPGGS | NYNENFK | TSRNFAK | WGQGT |
| | SVKMSCKAA (SEQ | (SEQ ID NO: | QRPGH | YT (SEQ | GKATFTA | (SEQ ID | PVTVSS |
| | ID NO: 1228) | 4) | GLEWI | ID NO: | DTSSSTT | NO: 1418) | (SEQ ID |
| | | | GD | 1016) | YMQLSSL | | NO: 398) |
| | | | (SEQ ID | | TSEDSAIY | | |
| | | | NO: | | FC (SEQ | | |
| | | | 1010) | | ID NO: | | |
| | | | | | 1189) | | |
| AH4524 | QIQLQQSGAELVRPGT | GYTFTNYW | IGWVK | IHPGGD | NYNEKFK | TSRNFAY | WGQGT |
| | SVKMSCKAA (SEQ | (SEQ ID NO: | QRPGH | YS (SEQ | GKATLTA | (SEQ ID | LVTVSA |
| | ID NO: 1228) | 4) | GLEWI | ID NO: | DTSSSTA | NO: 1419) | (SEQ ID |
| | | | GD | 1012) | YMSLSSL | | NO: 421) |
| | | | (SEQ ID | | TSEDSAIY | | |
| | | | NO: | | YC (SEQ | | |
| | | | 1010) | | ID NO: | | |
| | | | | | 1180) | | |
| AH4525 | QVQLQQSGAELVRPG | GYTFTNYW | IGWIK | IHPGGD | NYNEKFK | TGRNFA | WGQGT |
| | TSVKMSCKAA (SEQ | (SEQ ID NO: | QRPGH | YT (SEQ | GKATLTA | Y (SEQ ID | LVTVSS |
| | ID NO: 1326) | 4) | GLEWI | ID NO: | DTFSSTA | NO: 1395) | (SEQ ID |
| | | | GD | 1013) | YMQLSSL | | NO: 413) |
| | | | (SEQ ID | | TSEDSAIY | | |
| | | | NO: | | YC (SEQ | | |
| | | | 1007) | | ID NO: | | |
| | | | | | 1171) | | |
| D11 | QIQLQQPGAELVRPG | GYTFTDYA | MHWV | ISTYSG | IYNQKFK | ARGVTF | WGQGT |
| | VSLKISCKGS (SEQ | (SEQ ID NO: | RQSHA | DA (SEQ | GKATMT | DY (SEQ | TVTVSS |
| | ID NO: 1223) | 19) | KSLEW | ID NO: | VDKSSST | ID NO: | (SEQ ID |
| | | | IGV | 21) | AYLELAR | 23) | NO: 422) |
| | | | (SEQ ID | | LTSDDSAI | | |
| | | | NO: | | YYC (SEQ | | |
| | | | 1128) | | ID NO: | | |
| | | | | | 1033) | | |
| D17 | QIQLQQSGAELVRPGT | GYTFTNYW | LGWV | IYPGGG | NYNEKFK | ARVTPAS | WGQGT |
| | SVKISCKAS (SEQ | (SEQ ID NO: | KQRPG | YT (SEQ | GKATLTA | (SEQ ID | TLTVSA |
| | ID NO: 1227) | 4) | HGLEW | ID | DTSSSTA | NO: 8) | (SEQ ID |
| | | | IGD | NO: 6) | YMQLSSL | | NO: |
| | | | (SEQ ID | | TSEDSAV | | 1452) |
| | | | NO: | | YFC (SEQ | | |
| | | | 1075) | | ID NO: | | |
| | | | | | 1178) | | |
| D34 | QVQLQQSGAELVRPG | GYTFTDYA | MHWV | ISTYSG | SYNQKFK | ARGVTF | WGQGT |
| | VSVKISCKGS (SEQ | (SEQ ID NO: | KQSHA | DV (SEQ | GKATMT | DS (SEQ | TVTVSS |
| | ID NO: 1338) | 19) | KSLEW | ID NO: | VDKSSST | ID NO: | (SEQ ID |
| | | | IGV | 46) | AYMELA | 48) | NO: 422) |
| | | | (SEQ ID | | RLTSEDS | | |
| | | | NO: | | AIYYC | | |
| | | | 1126) | | (SEQ ID | | |
| | | | | | NO: 1389) | | |
| D36 | QVQLQQPGAELVRPG | GYTFTDYA | MHWV | ISTYSG | LYNQKFK | ARGVTF | WGQGT |
| | VSLKISCKGS (SEQ | (SEQ ID NO: | RQSHA | DA (SEQ | GKATMT | DY (SEQ | TLTVSS |
| | ID NO: 1319) | 19) | KSLEW | ID NO: | VDKSSST | ID NO: | (SEQ ID |
| | | | IGV | 21) | AYLELAR | 23) | NO: 425) |
| | | | (SEQ ID | | LTSEDSAI | | |
| | | | NO: | | YYC (SEQ | | |
| | | | 1128) | | ID NO: | | |
| | | | | | 1120) | | |
| D5 | QVQLQQSGAELVRPG | GYTFTNYW | LGWV | IYPGGG | NYNEKFK | ARVTPAS | WGQGT |
| | TSVKISCKAS (SEQ | (SEQ ID NO: | KQRPG | YT (SEQ | GKATLTA | (SEQ ID | SLTVSS |
| | ID NO: 1325) | 4) | HGLEW | ID | DTSSSTA | NO: 8) | (SEQ ID |
| | | | IGD | NO: 6) | YMQLSSL | | NO: 424) |
| | | | (SEQ ID | | TSEDSAV | | |
| | | | NO: | | YFC (SEQ | | |
| | | | 1075) | | ID NO: | | |
| | | | | | 1178) | | |
| BP003- | QVQLQQSGAELVRPG | GYTFTNYW | LGWV | IYPGGG | NYNEKFK | ARVTPAS | WGQGT |
| T2P1C4 | TSVKISCKAS (SEQ | (SEQ ID NO: | KQRPG | YT (SEQ | GKATLTA | (SEQ ID | TVTVSS |
| | ID NO: 1325) | 4) | HGLEW | ID | DTSSSTA | NO: 8) | (SEQ ID |
| | | | IGD | NO: 6) | YMQLSSL | | NO: 422) |
| | | | (SEQ ID | | TSEDSAV | | |
| | | | NO: | | YFC (SEQ | | |
| | | | 1075) | | ID NO: | | |
| | | | | | 1178) | | |
| BP003- | QIQLQQPGAELVRPGT | GYTFTNYW | LGWV | IYPGGG | NYNEKFK | ARVTPAS | WGQGT |
| T2P1D10 | SVKISCKAS (SEQ | (SEQ ID NO: | KQRPG | YT (SEQ | GKATLTA | (SEQ ID | SLTVSS |
| | ID NO: 1220) | 4) | HGLEW | ID | DTSSSTA | NO: 8) | (SEQ ID |
| | | | IGD | NO: 6) | YMQLSSL | | NO: 424) |
| | | | (SEQ ID | | TSEDSAV | | |
| | | | NO: | | YFC (SEQ | | |
| | | | 1075) | | ID NO: | | |
| | | | | | 1178) | | |
| BP003- | QAYLQQSGAELVRSG | GFNIKDYY | IHWVK | IDPDNG | EYAPKFQ | TVFWYG | WGQGT |
| T2P1D7 | ASVKLSCTAS (SEQ | (SEQ ID NO: | QRPEQ | ET (SEQ | GKATMT | NNYAGF | LVTVSA |
| | ID NO: 1197) | 977) | GLEWI | ID NO: | ADTSSNT | AY (SEQ | (SEQ ID |
| | | | GW | 1003) | AHLQLSS | ID NO: | NO: 421) |
| | | | (SEQ ID | | LTSEDTA | 1420) | |
| | | | NO: | | VYYC | | |
| | | | 1017) | | (SEQ ID | | |
| | | | | | NO: 946) | | |
| BP003- | QIQLQQSGAELVRPGT | GYTFTNYW | IGWVK | IHPGGG | NYNEKFK | TSRNFAY | WGQGT |
| T2P1E3 | SVKMSCKAA (SEQ | (SEQ ID NO: | QRPGH | YT (SEQ | GKATLTA | (SEQ ID | PVTVSS |
| | ID NO: 1228) | 4) | GLEWI | ID NO: | DTSSSTA | NO: 1419) | (SEQ ID |
| | | | GD | 1015) | YMQLSSL | | NO: 398) |
| | | | (SEQ ID | | TSEDSAIY | | |
| | | | NO: | | YC (SEQ | | |
| | | | 1010) | | ID NO: | | |
| | | | | | 1177) | | |
| BP003- | QVQLQQSGAELVRPG | GYTFTDYA | MHWV | ISTYSG | IYNQKFK | ARGVTF | WGQGT |
| T2P1D1 | VSLKISCKGS (SEQ | (SEQ ID NO: | RQSHA | DA (SEQ | GKATMT | DY (SEQ | TVTVSS |
| | ID NO: 1337) | 19) | KSLEW | ID NO: | VDKSSST | ID NO: | (SEQ ID |
| | | | IGV | 21) | AYLELAR | 23) | NO: 422) |
| | | | (SEQ ID | | LTSDDSAI | | |
| | | | NO: | | YYC (SEQ | | |
| | | | 1128) | | ID NO: | | |
| | | | | | 1033) | | |

TABLE 5B
Exemplary Clones-Light Chain Sequences

| ID | LFR1 | CDRL1 | LFR2 | CDRL2 | LFR3 | CDRL3 | LFR4 |
|---|---|---|---|---|---|---|---|
| AH04507 | DTTVTQSQKFMST | QNVGTN | VAWYQQK | SAS | YRYSGVPDRF | QQYNS | FGGGT |
| | SVGDRVSVTCKAS | (SEQ ID | PGQSPKAL | (SEQ ID | TGSGSGTDFT | YPYT | KLEIK |
| | (SEQ ID NO: 858) | NO: 12) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 959) |
| AH04522 | DTTVTQSQKFMST | QNVGTN | VAWYQQK | SAS | YRYSGVPDRF | QQYNS | FGGGT |
| | SVGDRVSVTCKA | (SEQ ID | PGQSPKAL | (SEQ ID | TGSGSGTDFT | YPYT | KLEIK |
| | G (SEQ ID NO: 856) | NO: 12) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 959) |
| AH04526 | DTTVTQSQKFMST | QNVGTN | VAWYQQK | SAS | YRYSGVPDRF | QQYNS | FGGGT |
| | SVGDRVSVTCKAS | (SEQ ID | PGQSPKAL | (SEQ ID | TGSGSGTDFT | YPYT | KLEMK |
| | (SEQ ID NO: 858) | NO: 12) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 961) |
| AH04527 | DVVMTQSQKFMS | QNVGTN | VAWYQQK | SAS | YRYSGVPDRF | QQYNS | FGGGT |
| | TSVGDRVSVTCKA | (SEQ ID | PGQSPKAL | (SEQ ID | TGSGSGTDFT | YPYT | KLEIK |
| | S (SEQ ID NO: 894) | NO: 12) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 959) |
| AH04734 | DIQMTQSTSSLSAS | QDIRNY | LNWYQQK | YTS | RLHSGVPSRFS | QQGNT | SGGGT |
| | LGDRVTISCRAS | (SEQ ID | PDGTVKLL | (SEQ ID | GSGSGTDYSL | LPPT | KLEIK |
| | (SEQ ID NO: 738) | NO: 1202) | IY (SEQ ID | NO: | TISNLEQEDIA | (SEQ ID | (SEQ ID |
| | | | NO: 1088) | 1495) | TYFC (SEQ ID | NO: | NO: |
| | | | | | NO: 1353) | 1253) | 1364) |
| AH04750 | DIQMTQTTSSLSA | QDISNY | LNWYQQK | YTS | RLHSGVPSRFS | QQGNT | FGGGT |
| | SLGDRVTISCRAS | (SEQ ID | PDGTVKLL | (SEQ ID | GSGSGTDYSL | LPPT | KLEMK |
| | (SEQ ID NO: 760) | NO: 27) | IY (SEQ ID | NO: | TISNLEQEDIA | (SEQ ID | (SEQ ID |
| | | | NO: 1088) | 1495) | TYFC (SEQ ID | NO: | NO: |
| | | | | | NO: 1353) | 1253) | 961) |
| AH05214 | DILLTQSPALMAA | SSISSSN | LHWYQQK | GTS | NLASGVPVRF | QQWSS | FGGGT |
| | SPGEKVTITCSVS | (SEQ ID | SETSPKPW | (SEQ ID | SGSGSGTSYSL | YPLT | KLEIK |
| | (SEQ ID NO: 698) | NO: 1382) | IY (SEQ ID | NO: 983) | TISSMEAEDA | (SEQ ID | (SEQ ID |
| | | | NO: 1078) | | ATYYC (SEQ | NO: | NO: |
| | | | | | ID NO: 1147) | 1277) | 959) |
| AH05247 | DIVMTQSQKFMST | QNVGTN | VAWYQQK | SAS | YRYSGVPDRF | QQYNS | FGGGT |
| | SVGDRVSVTCKAS | (SEQ ID | PGQSPKAL | (SEQ ID | TGSGSGTDFT | YPLT | KLEIK |
| | (SEQ ID NO: 821) | NO: 12) | IY (SEQ ID | NO: 13) | LTISNVQSEDL | (SEQ ID | (SEQ ID |
| | | | NO: 1425) | | AEYFC (SEQ | NO: 42) | NO: |
| | | | | | ID NO: 1483) | | 959) |
| AH05249 | DIVMTQTPKFLLV | QSVNND | VVWYQQK | YAS | NRYTGVPDRF | QQDYR | FGGGT |
| | SAGDRVTITCKAS | (SEQ ID | PGQSPKLL | (SEQ ID | TGSGYGTDFT | SPYT | KLEIK |
| | (SEQ ID NO: 830) | NO: 1309) | IY (SEQ ID | NO: | FTISTVQAEDL | (SEQ ID | (SEQ ID |
| | | | NO: 1447) | 1476) | AVYFC (SEQ | NO: | NO: |
| | | | | | ID NO: 1164) | 1249) | 959) |
| AH05251 | DVVMTQTPLSLPV | QSIVHSN | LEWYLQK | KVS | NRFSGVPDRF | FQGSH | FGGGT |
| | NLGDQASISCRSS | GNTY | PGQSPKLL | (SEQ ID | SGSGSGTDFTL | VPPT | KLEIK |
| | (SEQ ID NO: 898) | (SEQ ID | IY (SEQ ID | NO: | KISRVEAEDL | (SEQ ID | (SEQ ID |
| | | NO: 1299) | NO: 1071) | 1062) | GIYYC (SEQ ID | NO: | NO: |
| | | | | | NO: 1156) | 966) | 959) |
| AH05256 | DIQMTQTTSSLSA | QDISNY | LNWYQQK | HTS | RLQSGVPSRFT | QQSNS | FGGGT |
| | SLGDRVTISCRAS | (SEQ ID | PDGTVKLL | (SEQ ID | GSGSGTDYSL | LPPT | KLEIK |
| | (SEQ ID NO: 760) | NO: 27) | IY (SEQ ID | NO: | TISNLEQEDIA | (SEQ ID | (SEQ ID |
| | | | NO: 1088) | 1002) | TYFC (SEQ ID | NO: | NO: |
| | | | | | NO: 1355) | 1271) | 959) |
| AH05257 | DIVMTQSTSSLSAS | QDISNY | LSWYQQK | YTS | RLHSGVSSRFS | QQGHT | FGGGT |
| | LGDRVTISCRAS | (SEQ ID | PDGTIKLLI | (SEQ ID | GSGSGTDYSL | LPPT | KLEIK |
| | (SEQ ID NO: 828) | NO: 27) | Y (SEQ ID | NO: | TISNLEQEDFA | (SEQ ID | (SEQ ID |
| | | | NO: 1113) | 1495) | TYFC (SEQ ID | NO: | NO: |
| | | | | | NO: 1354) | 1252) | 959) |
| AH05258 | DIVMTQSHKFMST | QDVSTA | VAWYQQK | SAS | YRYTGVPDRF | QQHYS | FGSGT |
| | SVGDRVSITCKAS | (SEQ ID | PGQSPKLL | (SEQ ID | TGSGSGTDFTF | TPFT | KLEIK |
| | (SEQ ID NO: 800) | NO: 1209) | IY (SEQ ID | NO: 13) | TISSVQAEDLA | (SEQ ID | (SEQ ID |
| | | | NO: 1427) | | VYYC (SEQ ID | NO: | NO: |
| | | | | | NO: 1491) | 1260) | 965) |
| AH05259 | DIQMTQTPASLSA | ENIYSF | LEWFQQK | NAK | TLAEGVPSRFS | QHHYG | FGGGT |
| | SVGETVTITCRAS | (SEQ ID | QGKSPQLL | (SEQ ID | VSGSGTQFSL | IPYT | KLEIK |
| | (SEQ ID NO: 744) | NO: 928) | VY (SEQ ID | NO: 28) | KINSLQPEDFG | (SEQ ID | (SEQ ID |
| | | | NO: 1070) | | SYYC (SEQ ID | NO: | NO: |
| | | | | | NO: 1401) | 1214) | 959) |
| AH05268 | DVQMTQSPSSMY | QDINRY | LSWFQQK | RAN | RLVDGVPSRF | LQYDE | FGGGT |
| | ASLGERVTFTCKA | (SEQ ID | PGKSPKTL | (SEQ ID | SGSGSGQNYS | FPYT | KLEIK |
| | S (SEQ ID NO: 872) | NO: 1201) | IY (SEQ ID | NO: | LTISSLEYEDM | (SEQ ID | (SEQ ID |
| | | | NO: 1109) | 1345) | GIYYC (SEQ ID | NO: | NO: |
| | | | | | NO: 1357) | 1106) | 959) |
| AH05271 | DIQMTQTPASLAA | ENIYFS | LAWYQQK | NAN | TLEDGVPSRFS | KQAYD | FGGGT |
| | SVGETVTITCRAS | (SEQ ID | QGKSPQLL | (SEQ ID | GSGSGTQFSM | VPWT | KLEIK |
| | (SEQ ID NO: 743) | NO: 927) | IY (SEQ ID | NO: | KINNMQPEDT | (SEQ ID | (SEQ ID |
| | | | NO: 1068) | 1142) | ATYFC (SEQ | NO: | NO: |
| | | | | | ID NO: 1403) | 1058) | 959) |
| AH05274 | DIQMTQSQKFMST | QNVGTN | VAWYQQK | SAS | YRYSGVPDRF | QQYNS | FGGGT |
| | SVGDRVSVTCKAS | (SEQ ID | PGQSPKAL | (SEQ ID | TGSGSGTDFT | YPYT | KLEIK |
| | (SEQ ID NO: 734) | NO: 12) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 959) |
| AH05280 | DIVMTQTPKFLLV | QSVNND | VLWYQQK | YAS | NRYTGVPDRF | QQAY | FGGGT |
| | SAGDRVTITCKAS | (SEQ ID | PGQSPKLL | (SEQ ID | TGSGYGTDFT | WSPYT | KLEIK |
| | (SEQ ID NO: 830) | NO: 1309) | IY (SEQ ID | NO: | FTISTVQAEDL | (SEQ ID | (SEQ ID |
| | | | NO: 1435) | 1476) | AVYFC (SEQ | NO: | NO: |
| | | | | | ID NO: 1164) | 1248) | 959) |
| AH05285 | DIQMTQTTSSLSA | QDISNY | LNWYQQK | YTS | RLHSGVPSRFS | QQGNT | FGGGT |
| | SLGDRVTISCRAS | (SEQ ID | SDGTVKLL | (SEQ ID | GSGSGTDYSL | LPPT | KLEMK |
| | (SEQ ID NO: 760) | NO: 27) | IY (SEQ ID | NO: | TISNLEQEDIA | (SEQ ID | (SEQ ID |
| | | | NO: 1089) | 1495) | TYFC (SEQ ID | NO: | NO: |
| | | | | | NO: 1353) | 1253) | 961) |
| AH05286 | DIVLTQSPPTLSLS | QDVNTA | VAWYQQK | WAS | TRHTGVPSRFS | QQHYS | FGGGT |
| | PGERVTLSCKAS | (SEQ ID | PGQAPRLL | (SEQ ID | GSGSGTDFTL | SPWT | KLEIK |
| | (SEQ ID NO: 771) | NO: 1206) | IY (SEQ ID | NO: | TISSLQPEDFA | (SEQ ID | (SEQ ID |
| | | | NO: 1424) | 1450) | TYYC (SEQ ID | NO: | NO: |
| | | | | | NO: 1414) | 1258) | 959) |
| AH4501 | DVVMTQTQKFTS | QNVGTN | VAWYQQK | SAS | YRYSGVPDRF | QQYNS | FGGGT |
| | TSVGDRVSVTCKA | (SEQ ID | PGQSPKAL | (SEQ ID | TGSGSGTDFT | YPYT | KLEIK |
| | S (SEQ ID NO: 911) | NO: 12) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 959) |
| AH4502 | DIVMTQSQKFMST | QNVGIN | VAWYQQK | SAS | YRYSGVPDRF | QQYNS | FGGGT |
| | SVGDRVSVTCKAS | (SEQ ID | PGQSPKAL | (SEQ ID | TGSGSGTDFT | YPYT | KLEIK |
| | (SEQ ID NO: 821) | NO: 1243) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 959) |
| AH4503 | DIQMTQSQKFMST | QNVGTN | VAWYQQK | SAS | YRYSGVPDRF | QQYNS | FGGGT |
| | SVGDRVSVTCKAS | (SEQ ID | PGQSPKAL | (SEQ ID | TGSGSGTDFT | YPWT | KLEIK |
| | (SEQ ID NO: 734) | NO: 12) | IY (SEQ ID | NO: 13) | LTISNVQSEDL | (SEQ ID | (SEQ ID |
| | | | NO: 1425) | | AEYFC (SEQ | NO: 36) | NO: |
| | | | | | ID NO: 1483) | | 959) |
| AH4505 | DTTVTQSQKFMST | QNVGTN | VAWYQQK | SAS | YRYSGVPDRF | QQYNS | FGGGT |
| | SVGDRVSVTCKAS | (SEQ ID | PGQSPKAL | (SEQ ID | TGSGSGTDFT | YPYT | KLEMK |
| | (SEQ ID NO: 858) | NO: 12) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 961) |
| AH4509 | DTTVTQSQRFMST | QNVGTN | VAWYQQK | SAS | YRYSGVPDRF | QQYNN | FGGGT |
| | SVGDRVSVTCKAS | (SEQ ID | PGQSPKPLI | (SEQ ID | TGSGSGTDFT | SPLT | KLEIK |
| | (SEQ ID NO: 863) | NO: 12) | Y (SEQ ID | NO: 13) | LTINNVQSEDL | (SEQ ID | (SEQ ID |
| | | | NO: 1429) | | AEYFC (SEQ | NO: | NO: |
| | | | | | ID NO: 1480) | 1282) | 959) |
| AH4511 | DIVMSQSQKFMST | QNVGTN | VAWYQQK | SAS | YRYSGVPDRF | QQYNS | FGGGT |
| | SVGDRVSVTCKAS | (SEQ ID | PGQSPKAL | (SEQ ID | TGSGSGTDFT | YPYT | KLEIK |
| | (SEQ ID NO: 793) | NO: 12) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 959) |
| AH4518 | DILLTQSQKFMST | QNVGTN | VAWYQQK | SAS | YRYSGVPDRF | QQYNS | FGGGT |
| | SVGDRVSVTCKAS | (SEQ ID | PGQSPKAL | (SEQ ID | TGSGSGTDFT | YPYT | KLEIK |
| | (SEQ ID NO: 700) | NO: 12) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 959) |
| AH4523 | DIVLTQSQKFMST | QNVGTN | VAWYQQK | SAS | YRYSGVPDRF | QQYNS | FGGGT |
| | SVGDRVSVTCKAS | (SEQ ID | PGQSPKAL | (SEQ ID | TGSGSGTDFT | YPYT | KLEIK |
| | (SEQ ID NO: 773) | NO: 12) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 959) |
| AH4524 | DIVMTQSQKFMST | QNVGTN | VAWYQQK | SAS | YRYSGVPDRF | QQYNS | FGGGT |
| | SVGDRVSVTCKAS | (SEQ ID | PGQSPKAL | (SEQ ID | TGSGSGTDFT | YPYT | KLEIK |
| | (SEQIDNO: 821) | NO: 12) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 959) |
| AH4525 | DNVLTQSQKFMST | QNVGTN | VAWYQQK | SAS | YRYSGVPDRF | QQYNS | FGGGT |
| | SVGDRVSVTCKAS | (SEQ ID | PGQSPKAL | (SEQ ID | TGSGSGTDFT | YPYT | KLEMK |
| | (SEQ ID NO: 837) | NO: 12) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 961) |
| D11 | DIQMTQTTSSLSA | QDISNY | LEWYQQK | NAK | TLAEGVPSRFS | QHHYD | FGGGT |
| | SLGDRVTITCRAS | (SEQ ID | QGKSPQLL | (SEQ ID | GSGSGTQFSL | TPYT | KLEIK |
| | (SEQ ID NO: 763) | NO: 27) | VY (SEQ ID | NO: 28) | KINSLQPEDFG | (SEQ ID | (SEQ ID |
| | | | NO: 1072) | | SYYC (SEQ ID | NO: 30) | NO: |
| | | | | | NO: 1398) | | 959) |
| D17 | DILLTQSQKFMST | QNVGTN | VAWYQQK | SAS | YRYSGVPDRF | QQYNS | FGGGT |
| | SVGDRVSVTCKAS | (SEQ ID | PGQSPKAL | (SEQ ID | TGSGSGTDFT | YPLT | KLEIK |
| | (SEQ ID NO: 700) | NO: 12) | IY (SEQ ID | NO: 13) | LTISNVQSEDL | (SEQ ID | (SEQ ID |
| | | | NO: 1425) | | AEYFC (SEQ | NO: 42) | NO: |
| | | | | | ID NO: 1483) | | 959) |
| D34 | DIQMTQSPASLSA | ENSYSY | LEWYQQK | NAK | TLAEGVPSRFS | QHHYG | FGGGT |
| | SVGETVTITCRAS | (SEQ ID | QGKSPQLL | (SEQ ID | GSGSGTQFSL | TPYT | KLEIK |
| | (SEQ ID NO: 720) | NO: 52) | VY (SEQ ID | NO: 28) | KINSLQPEDFG | (SEQ ID | (SEQ ID |
| | | | NO: 1072) | | TYYC (SEQ ID | NO: 54) | NO: |
| | | | | | NO: 1399) | | 959) |
| D36 | DVVMTQSPASLSA | ENSYSY | LEWYQQK | NAK | TLAEGVPSRFS | QHHYG | FGGGT |
| | SVGETVTITCRAS | (SEQ ID | QGKSPQLL | (SEQ ID | GSGSGTQFSL | TPYT | KLEMK |
| | (SEQ ID NO: 890) | NO: 52) | VY (SEQ ID | NO: 28) | KINSLQPEDFG | (SEQ ID | (SEQ ID |
| | | | NO: 1072) | | TYYC (SEQ ID | NO: 54) | NO: |
| | | | | | NO: 1399) | | 961) |
| D5 | DIVMTQSQKFMST | QNVGTN | VAWYQQK | SAS | YRYSGVPDRF | QQYNS | FGGGT |
| | SVGDRVSVTCKAS | (SEQ ID | PGQSPKAL | (SEQ ID | TGSGSGTDFT | YPYT | KLEMK |
| | (SEQ ID NO: 821) | NO: 12) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 961) |
| BP003- | DIQMTQSQKFMSA | QNVGTN | VAWYQQK | SAS | YRYSGVPDRF | QQYNS | FGGGT |
| T2P1C4 | SVGDRVSVTCKAS | (SEQ ID | PGQSPKAL | (SEQ ID | TGSGSGTDFT | YPYT | KLEMK |
| | (SEQ ID NO: 729) | NO: 12) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 961) |
| BP003- | DIVITQSQKFMSTS | QNVGTN | VAWYQQK | SAS | YRYSGVPDRL | QQYNR | FGGGT |
| T2P1D10 | VGDRVSVTCKAS | (SEQ ID | PGQSPKAL | (SEQ ID | TGSGSGTDFT | YPYT | KLEIK |
| | (SEQ ID NO: 766) | NO: 12) | IY (SEQ ID | NO: 13) | LTISNVQSEDL | (SEQ ID | (SEQ ID |
| | | | NO: 1425) | | AEYFC (SEQ | NO: | NO: |
| | | | | | ID NO: 1489) | 1285) | 959) |
| BP003- | DVVLTQTPLSLPV | QSIVHSN | LEWYLQK | KVS | NRFSGVPDRF | FQGSH | FGGGT |
| T2P1D7 | SLGDQASISCRSS | GNTY | PGQSPKLL | (SEQ ID | SGSGSGTDFTL | VPPT | KLEIK |
| | (SEQ ID NO: 874) | (SEQ ID | IY (SEQ ID | NO: | KISKVEAEDL | (SEQ ID | (SEQ ID |
| | | NO: 1299) | NO: 1071) | 1062) | GVYYC (SEQ | NO: | NO: |
| | | | | | ID NO: 1154) | 966) | 959) |
| BP003- | DIVLTQSQKFMST | QNVGTN | VAWYQQK | SAS | YRYSGVPDRF | QQYNS | FGGGT |
| T2P1E3 | SVGDRVSVTCKAS | (SEQ ID | PGQSPKAL | (SEQ ID | TGSGSGTDFT | YPYT | KLEIK |
| | (SEQ ID NO: 773) | NO: 12) | IY (SEQ ID | NO: 13) | LTISNVQSEDL | (SEQ ID | (SEQ ID |
| | | | NO: 1425) | | AEYFC (SEQ | NO: 15) | NO: |
| | | | | | ID NO: 1483) | | 959) |
| BP003- | DVVMTQSPASLSA | ENSYSY | LEWYQQK | NAK | TLAEGVPSRFS | QHHYG | FGGGT |
| T2P1D1 | SVGETVTITCRAS | (SEQ ID | QGKSPQLL | (SEQ ID | GSGSGTQFSL | TPYT | KLEIK |
| | (SEQ ID NO: 890) | NO: 52) | VY (SEQ ID | NO: 28) | KINSLQPEDFG | (SEQ ID | (SEQ ID |
| | | | NO: 1072) | | TYYC (SEQ ID | NO: 54) | NO: |
| | | | | | NO: 1399) | | 959) |

TABLE 6
Heavy Chain CDRs

| Combination | CDRH1 | CDRH2 | CDRH3 |
|---|---|---|---|
| 1 | GDTFSSYV (SEQ ID NO: 167) | FNPYSDDI (SEQ ID NO: 242) | GSGYDGYYDWFAC (SEQ |
| | | | ID NO: 315) |
| 2 | GFAFSSYD (SEQ ID NO: 154) | ISSGGGST (SEQ ID NO: 227) | ARNYRSWFAY (SEQ ID NO: |
| | | | 300) |
| 3 | GFNIKDYY (SEQ ID NO: 977) | IDPDNGET (SEQ ID NO: | TVFWYGNNYAGFAY (SEQ |
| | | 1003) | ID NO: 1420) |
| 4 | GFNIKDYY (SEQ ID NO: 977) | IDPENGDT (SEQ ID NO: | NVITTATTWFAY (SEQ ID |
| | | 1004) | NO: 1165) |
| 5 | GFSLSTSGMG (SEQ ID NO: | IYWDDDK (SEQ ID NO: 515) | ARRAGDYGNPFPY (SEQ ID |
| | 492) | | NO: 557) |
| 6 | GFSLSTSGMS (SEQ ID NO: | IWWNDDK (SEQ ID NO: 238) | ARIGGNDGYYWYFDV |
| | 163) | | (SEQ ID NO: 311) |
| 7 | GFSLTSYG (SEQ ID NO: 161) | IWAGGST (SEQ ID NO: 234) | ARGAYFDY (SEQ ID NO: |
| | | | 307) |
| 8 | GFSLTSYG (SEQ ID NO: 161) | IWAGGST (SEQ ID NO: 234) | AREGTGPWFAY (SEQ ID |
| | | | NO: 322) |
| 9 | GFTFNDAW (SEQ ID NO: | IRSKANNHAT (SEQ ID NO: | TNYGSNPLDY (SEQ ID NO: |
| | 172) | 239) | 321) |
| 10 | GFTFNTYA (SEQ ID NO: 182) | IRSKSNNYVT (SEQ ID NO: | CERVRRCV (SEQ ID NO: |
| | | 257) | 343) |
| 11 | GFTFSDAW (SEQ ID NO: | IRSKANNHAT (SEQ ID NO: | TPQFAY (SEQ ID NO: 312) |
| | 164) | 239) | |
| 12 | GFTFSDYY (SEQ ID NO: 160) | ISNGGGST (SEQ ID NO: 233) | ASPLGYDGFAY (SEQ ID |
| | | | NO: 306) |
| 13 | GFTFSGFW (SEQ ID NO: 176) | INSDGSAI (SEQ ID NO: 249) | MRYGSSYWYFD (SEQ ID |
| | | | NO: 328) |
| 14 | GFTFSNYR (SEQ ID NO: 183) | ITVKSDNYGA (SEQ ID NO: | SRWFAY (SEQ ID NO: 344) |
| | | 258) | |
| 15 | GFTFSNYR (SEQ ID NO: 183) | ITVKSDNYGA (SEQ ID NO: | SRLFAY (SEQ ID NO: 534) |
| | | 258) | |
| 16 | GFTFSNYW (SEQ ID NO: | IRLKSNNYAT (SEQ ID NO: | TGSDY (SEQ ID NO: 314) |
| | 166) | 241) | |
| 17 | GFTFSNYW (SEQ ID NO: | IRLKSNNYAT (SEQ ID NO: | TRIYDSGSSYTWYFDV |
| | 166) | 241) | (SEQ ID NO: 543) |
| 18 | GFTFSSFG (SEQ ID NO: 491) | ISGGGGTI (SEQ ID NO: 514) | ARWRGGYFDY (SEQ ID |
| | | | NO: 555) |
| 19 | GFTFSSYA (SEQ ID NO: 151) | ISSGGST (SEQ ID NO: 224) | ARGEIWGKAWFAY (SEQ |
| | | | ID NO: 297) |
| 20 | GFTFSSYA (SEQ ID NO: 151) | ISSGGSYT (SEQ ID NO: 237) | ARGGMITPFAY (SEQ ID |
| | | | NO: 310) |
| 21 | GFTFSSYA (SEQ ID NO: 151) | ISSGGST (SEQ ID NO: 224) | ARGYGSSFAY (SEQ ID NO: |
| | | | 339) |
| 22 | GFTFSSYA (SEQ ID NO: 151) | ISSGGSYT (SEQ ID NO: 237) | ARRIGYDGGGSWFAY (SEQ |
| | | | ID NO: 342) |
| 23 | GFTFSSYG (SEQ ID NO: 165) | INSNGGST (SEQ ID NO: 240) | ASHYDEGY (SEQ ID NO: |
| | | | 313) |
| 24 | GFTFSSYG (SEQ ID NO: 165) | INSNGGST (SEQ ID NO: 240) | ARGGNPY (SEQ ID NO: 327) |
| 25 | GFTFSSYG (SEQ ID NO: 165) | ISSGGSYT (SEQ ID NO: 237) | ARHYYDYDYWYFDV (SEQ |
| | | | ID NO: 330) |
| 26 | GFTFSSYG (SEQ ID NO: 165) | INSNGGST (SEQ ID NO: 240) | ASLAY (SEQ ID NO: 548) |
| 27 | GFTFSSYT (SEQ ID NO: 485) | ISSGGGYT (SEQ ID NO: 508) | TRVSAKYFDV (SEQ ID NO: |
| | | | 539) |
| 28 | GFTFSSYW (SEQ ID NO: 177) | IRLKSDNYAT (SEQ ID NO: | TRYYYGES (SEQ ID NO: |
| | | 250) | 329) |
| 29 | GFTFSSYW (SEQ ID NO: 177) | IRLKSDNYAT (SEQ ID NO: | TCDYDGGAWFAY (SEQ ID |
| | | 250) | NO: 332) |
| 30 | GFTFTDYY (SEQ ID NO: 180) | IRNKANGYTT (SEQ ID NO: | ARDKRITTVEAWFAY (SEQ |
| | | 255) | ID NO: 338) |
| 31 | GFTFTDYY (SEQ ID NO: 180) | IRNKANGYTT (SEQ ID NO: | ARDGEVRRALAY (SEQ ID |
| | | 255) | NO: 537) |
| 32 | GFTFTDYY (SEQ ID NO: 180) | IRNKANGYTT (SEQ ID NO: | ARGWGNWFAY (SEQ ID |
| | | 255) | NO: 554) |
| 33 | GLTFSSYG (SEQ ID NO: 171) | ISSGGSYI (SEQ ID NO: 244) | ARQDDGYYRIFDY (SEQ ID |
| | | | NO: 320) |
| 34 | GYAFSSYW (SEQ ID NO: | IYPGDGDT (SEQ ID NO: 248) | ARSGYRYDAVFAY (SEQ ID |
| | 175) | | NO: 326) |
| 35 | GYAFTNYL (SEQ ID NO: | INPGSGGT (SEQ ID NO: 228) | ARKGSLTGVLAY (SEQ ID |
| | 155) | | NO: 301) |
| 36 | GYAFTNYW (SEQ ID NO: | IYPGGGYT (SEQ ID NO: 6) | ARVTPAS (SEQ ID NO: 8) |
| | 984) | | |
| 37 | GYIFTNYW (SEQ ID NO: 156) | IDPSDSET (SEQ ID NO: 229) | ARRGLRAWFAY (SEQ ID |
| | | | NO: 302) |
| 38 | GYSFTKNG (SEQ ID NO: 168) | INTYTGEP (SEQ ID NO: 232) | AREPKTLDY (SEQ ID NO: |
| | | | 316) |
| 39 | GYSITSDYA (SEQ ID NO: | ISYSGST (SEQ ID NO: 506) | ARSRGNYFDY (SEQ ID NO: |
| | 482) | | 532) |
| 40 | GYSITSGYS (SEQ ID NO: | IHYSGST (SEQ ID NO: 245) | ARDPPFAY (SEQ ID NO: |
| | 173) | | 323) |
| 41 | GYTFNSHW (SEQ ID NO: | IDPYDSET (SEQ ID NO: 235) | ARPYDYDGFAY (SEQ ID |
| | 169) | | NO: 318) |
| 42 | GYTFSNY (SEQ ID NO: 985) | DIHPGGGYINYNEKFTG | SRNFAN (SEQ ID NO: 1378) |
| | | (SEQ ID NO: 695) | |
| 43 | GYTFSNYW (SEQ ID NO: | IHPGGGYI (SEQ ID NO: 1014) | VSRNFAN (SEQ ID NO: |
| | 986) | | 1445) |
| 44 | GYTFSNYW (SEQ ID NO: | ILPGSGFT (SEQ ID NO: 1021) | ARGGTSVVHFDY (SEQ ID |
| | 986) | | NO: 605) |
| 45 | GYTFSNYW (SEQ ID NO: | ILPGSGFT (SEQ ID NO: 1021) | ARGGTSVVHFDS (SEQ ID |
| | 986) | | NO: 604) |
| 46 | GYTFSNYW (SEQ ID NO: | ILPGSGFT (SEQ ID NO: 1021) | ARGGTSVVHFDFDY (SEQ |
| | 986) | | ID NO: 603) |
| 47 | GYTFSNYW (SEQ ID NO: | ILPGSGFT (SEQ ID NO: 1021) | ARGGTSVVHFDS (SEQ ID |
| | 986) | | NO: 604) |
| 48 | GYTFSNYW (SEQ ID NO: | ILPGSGFT (SEQ ID NO: 1021) | ARGGTSVVHFDY (SEQ ID |
| | 986) | | NO: 605) |
| 49 | GYTFSNYW (SEQ ID NO: | ILPGSGYT (SEQ ID NO: 1022) | ARGGTSFVHFDY (SEQ ID |
| | 986) | | NO: 602) |
| 50 | GYTFSSYW (SEQ ID NO: | ILPGSGST (SEQ ID NO: 254) | ARRTYYGNAWFAY (SEQ |
| | 179) | | ID NO: 337) |
| 51 | GYTFSSYW (SEQ ID NO: | ILPGSGST (SEQ ID NO: 254) | ARSAHRYDAWFAY (SEQ |
| | 179) | | ID NO: 551) |
| 52 | GYTFTDYA (SEQ ID NO: 19) | ISTYSGDV (SEQ ID NO: 46) | ARGVTFDS (SEQ ID NO: 48) |
| 53 | GYTFTDYA (SEQ ID NO: 19) | ISTYSGDA (SEQ ID NO: 21) | ARGVTFDY (SEQ ID NO: 23) |
| 54 | GYTFTDYA (SEQ ID NO: 19) | IITYSGDA (SEQ ID NO: 1020) | AXGVTFDY (SEQ ID NO: |
| | | | 619) |
| 55 | GYTFTDYA (SEQ ID NO: 19) | ISTYSGDV (SEQ ID NO: 46) | ARGVTFDS (SEQ ID NO: 48) |
| 56 | GYTFTDYA (SEQ ID NO: 19) | ISTYSGDA (SEQ ID NO: 21) | ARGVTFDY (SEQ ID NO: 23) |
| 57 | GYTFTDYA (SEQ ID NO: 19) | IXTYSGDV (SEQ ID NO: | ARGVTFDS (SEQ ID NO: 48) |
| | | 1029) | |
| 58 | GYTFTDYA (SEQ ID NO: 19) | ISTYSGDA (SEQ ID NO: 21) | ARGVTFDY (SEQ ID NO: 23) |
| 59 | GYTFTDYA (SEQ ID NO: 19) | ISTYSGDA (SEQ ID NO: 21) | ARGVTFDY (SEQ ID NO: 23) |
| 60 | GYTFTDYA (SEQ ID NO: 19) | ISTYSGDV (SEQ ID NO: 46) | ARGVTFDS (SEQ ID NO: 48) |
| 61 | GYTFTDYA (SEQ ID NO: 19) | ISTYSGDA (SEQ ID NO: 21) | ARGVTFDY (SEQ ID NO: 23) |
| 62 | GYTFTDYE (SEQ ID NO: 174) | IHPGSGGT (SEQ ID NO: 247) | TRNGNGNWYFDV (SEQ ID |
| | | | NO: 325) |
| 63 | GYTFTDYE (SEQ ID NO: 174) | IHPGSGGT (SEQ ID NO: 247) | TRSDYGSSYEFAY (SEQ ID |
| | | | NO: 531) |
| 64 | GYTFTDYG (SEQ ID NO: | ISTYSGDV (SEQ ID NO: 46) | ARGVTFDS (SEQ ID NO: 48) |
| | 987) | | |
| 65 | GYTFTDYS (SEQ ID NO: 487) | INTETGEP (SEQ ID NO: 225) | ASFYYGNFAYYFDY (SEQ |
| | | | ID NO: 542) |
| 66 | GYTFTDYY (SEQ ID NO: | IYPGSGNT (SEQ ID NO: 511) | ARVYSGFDV (SEQ ID NO: |
| | 489) | | 549) |
| 67 | GYTFTKY (SEQ ID NO: 989) | DIHPGGGYINYNEKFTG | SRNFAN (SEQ ID NO: 1378) |
| | | (SEQ ID NO: 695) | |
| 68 | GYTFTNFY (SEQ ID NO: 486) | INPSNGGT (SEQ ID NO: 231) | TRSYYDYDWYFDV (SEQ |
| | | | ID NO: 540) |
| 69 | GYTFTNHH (SEQ ID NO: | INPYNDYT (SEQ ID NO: 243) | ADGDYYFDY (SEQ ID NO: |
| | 170) | | 319) |
| 70 | GYTFTNY (SEQ ID NO: 990) | DIHPGGDYSNYNEKFKG | SRNFAY (SEQ ID NO: 1379) |
| | | (SEQ ID NO: 692) | |
| 71 | GYTFTNY (SEQ ID NO: 990) | DIHPGGGYTNYNEKFKG | SRNFAY (SEQ ID NO: 1379) |
| | | (SEQ ID NO: 696) | |
| 72 | GYTFTNY (SEQ ID NO: 990) | DIHPGGSYTNYNENFKG | SRNFAK (SEQ ID NO: 1377) |
| | | (SEQ ID NO: 697) | |
| 73 | GYTFTNY (SEQ ID NO: 990) | DIHPGGGYIDYNEKFTG | SRNFAK (SEQ ID NO: 1377) |
| | | (SEQ ID NO: 694) | |
| 74 | GYTFTNY (SEQ ID NO: 990) | DIHPGGDYTNYNEKFKG | GRNFAY (SEQ ID NO: 982) |
| | | (SEQ ID NO: 693) | |
| 75 | GYTFTNY (SEQ ID NO: 990) | DFYPGGDYINYNEKFKG | SRNFAY (SEQ ID NO: 1379) |
| | | (SEQ ID NO: 691) | |
| 76 | GYTFTNYG (SEQ ID NO: | INTYTGEP (SEQ ID NO: 232) | ASYYDSTYVGFAY (SEQ ID |
| | 159) | | NO: 305) |
| 77 | GYTFTNYW (SEQ ID NO: 4) | IHPGGGYT (SEQ ID NO: | TSRNFAY (SEQ ID NO: |
| | | 1015) | 1419) |
| 78 | GYTFTNYW (SEQ ID NO: 4) | IYPGGGYT (SEQ ID NO: 6) | ARVSPAS (SEQ ID NO: 615) |
| 79 | GYTFTNYW (SEQ ID NO: 4) | IYPGGGYT (SEQ ID NO: 6) | ARVTPAS (SEQ ID NO: 8) |
| 80 | GYTFTNYW (SEQ ID NO: 4) | IHPGGDYT (SEQ ID NO: | TGRNFAY (SEQ ID NO: |
| | | 1013) | 1395) |
| 81 | GYTFTNYW (SEQ ID NO: 4) | IHPGGDYS (SEQ ID NO: | TSRNFAY (SEQ ID NO: |
| | | 1012) | 1419) |
| 82 | GYTFTNYW (SEQ ID NO: 4) | IHPGGGYI (SEQ ID NO: 1014) | VSRNFAN (SEQ ID NO: |
| | | | 1445) |
| 83 | GYTFTNYW (SEQ ID NO: 4) | INPGGGYT (SEQ ID NO: | TSRNFAY (SEQ ID NO: |
| | | 1023) | 1419) |
| 84 | GYTFTNYW (SEQ ID NO: 4) | IYPGGGYT (SEQ ID NO: 6) | VRVTPAS (SEQ ID NO: |
| | | | 1443) |
| 85 | GYTFTNYW (SEQ ID NO: 4) | FYPGGDYI (SEQ ID NO: 973) | TSRNFAY (SEQ ID NO: |
| | | | 1419) |
| 86 | GYTFTNYW (SEQ ID NO: 4) | IYPGGGYT (SEQ ID NO: 6) | VRVTPAS (SEQ ID NO: |
| | | | 1443) |
| 87 | GYTFTNYW (SEQ ID NO: 4) | IHPGGGYT (SEQ ID NO: | TSRNFAY (SEQ ID NO: |
| | | 1015) | 1419) |
| 88 | GYTFTNYW (SEQ ID NO: 4) | INPGGGYT (SEQ ID NO: | TSRNFAY (SEQ ID NO: |
| | | 1023) | 1419) |
| 89 | GYTFTNYW (SEQ ID NO: 4) | IYPGGGYA (SEQ ID NO: | ARVTPAS (SEQ ID NO: 8) |
| | | 1035) | |
| 90 | GYTFTNYW (SEQ ID NO: 4) | IHPGGGYI (SEQ ID NO: 1014) | VSRNFAK (SEQ ID NO: |
| | | | 1444) |
| 91 | GYTFTNYW (SEQ ID NO: 4) | IHPGGGYT (SEQ ID NO: | TSRNFAY (SEQ ID NO: |
| | | 1015) | 1419) |
| 92 | GYTFTNYW (SEQ ID NO: 4) | IHPGGDYS (SEQ ID NO: | TSRNFAY (SEQ ID NO: |
| | | 1012) | 1419) |
| 93 | GYTFTNYW (SEQ ID NO: 4) | IYPGGGYT (SEQ ID NO: 6) | ARVTPAS (SEQ ID NO: 8) |
| 94 | GYTFTNYW (SEQ ID NO: 4) | IHPGGSYT (SEQ ID NO: | TSRNFAK (SEQ ID NO: |
| | | 1016) | 1418) |
| 95 | GYTFTNYW (SEQ ID NO: 4) | IYPGGGYT (SEQ ID NO: 6) | ARVTPAS (SEQ ID NO: 8) |
| 96 | GYTFTNYW (SEQ ID NO: 4) | IYPGGGYT (SEQ ID NO: 6) | ARVTPAS (SEQ ID NO: 8) |
| 97 | GYTFTNYY (SEQ ID NO: | INPTNGGT (SEQ ID NO: 513) | TRGMAYRYDGAGWFAY |
| | 490) | | (SEQ ID NO: 552) |
| 98 | GYTFTRFW (SEQ ID NO: | INPSTDYT (SEQ ID NO: 1027) | ARGTVVDY (SEQ ID NO: |
| | 991) | | 607) |
| 99 | GYTFTRYW (SEQ ID NO: | INPSTGYT (SEQ ID NO: 230) | ARWGNFDY (SEQ ID NO: |
| | 992) | | 617) |
| 100 | GYTFTRYW (SEQ ID NO: | INPRTDYT (SEQ ID NO: | ARHGYFDY (SEQ ID NO: |
| | 992) | 1025) | 611) |
| 101 | GYTFTRYW (SEQ ID NO: | INPSSDYT (SEQ ID NO: 1026) | ARGTVVVDY (SEQ ID NO: |
| | 992) | | 608) |
| 102 | GYTFTRYW (SEQ ID NO: | INPSTDYT (SEQ ID NO: 1027) | VRSPILDY (SEQ ID NO: |
| | 992) | | 1442) |
| 103 | GYTFTRYW (SEQ ID NO: | INPRTDYT (SEQ ID NO: | ARHGYFDY (SEQ ID NO: |
| | 992) | 1025) | 611) |
| 104 | GYTFTRYY (SEQ ID NO: 488) | INPSNGGT (SEQ ID NO: 231) | TKGGFYDFFAY (SEQ ID |
| | | | NO: 547) |
| 105 | GYTFTSST (SEQ ID NO: 484) | INPSSGYT (SEQ ID NO: 236) | VRHYYFDY (SEQ ID NO: |
| | | | 538) |
| 106 | GYTFTSYN (SEQ ID NO: 178) | IYPGNGDT (SEQ ID NO: 253) | TRSGGNLWFAY (SEQ ID |
| | | | NO: 336) |
| 107 | GYTFTSYT (SEQ ID NO: 162) | INPSSGYT (SEQ ID NO: 236) | ARWDGAY (SEQ ID NO: |
| | | | 309) |
| 108 | GYTFTSYT (SEQ ID NO: 162) | INPSSGYT (SEQ ID NO: 236) | AREGKNWYFDV (SEQ ID |
| | | | NO: 334) |
| 109 | GYTFTSYT (SEQ ID NO: 162) | INPSSGYT (SEQ ID NO: 236) | ARSGLRQAWFAY (SEQ ID |
| | | | NO: 533) |
| 110 | GYTFTSYV (SEQ ID NO: 153) | INPYNDGT (SEQ ID NO: 226) | ARDGYYVGPAY (SEQ ID |
| | | | NO: 299) |
| 111 | GYTFTSYW (SEQ ID NO: | IYPGSGST (SEQ ID NO: 251) | TRSGVEGLLHWYFD (SEQ |
| | 157) | | ID NO: 1416) |
| 112 | GYTFTSYW (SEQ ID NO: | IYPGSGST (SEQ ID NO: 251) | TRWITTDHYFDY (SEQ ID |
| | 157) | | NO: 1417) |
| 113 | GYTFTSYW (SEQ ID NO: | IYPGSGST (SEQ ID NO: 251) | TRSGVEGLLHWYFD (SEQ |
| | 157) | | ID NO: 1416) |
| 114 | GYTFTSYW (SEQ ID NO: | IDPYDSET (SEQ ID NO: 235) | AREASYYYGNAWFA (SEQ |
| | 157) | | ID NO: 601) |
| 115 | GYTFTSYW (SEQ ID NO: | INPSNGRT (SEQ ID NO: 510) | ARQLAAY (SEQ ID NO: 614) |
| | 157) | | |
| 116 | GYTFTSYW (SEQ ID NO: | INPSTGYT (SEQ ID NO: 230) | ARLDYYGSSRGFAY (SEQ |
| | 157) | | ID NO: 303) |
| 117 | GYTFTSYW (SEQ ID NO: | IDPYDSET (SEQ ID NO: 235) | ARSPAYYGNLWFAY (SEQ |
| | 157) | | ID NO: 308) |
| 118 | GYTFTSYW (SEQ ID NO: | IDPSDSET (SEQ ID NO: 229) | ANWAWFAY (SEQ ID NO: |
| | 157) | | 317) |
| 119 | GYTFTSYW (SEQ ID NO: | IDPSDSYT (SEQ ID NO: 246) | AREEITAWFAY (SEQ ID |
| | 157) | | NO: 324) |
| 120 | GYTFTSYW (SEQ ID NO: | IYPGSGST (SEQ ID NO: 251) | TRSGVEGLLHWYFDV (SEQ |
| | 157) | | ID NO: 331) |
| 121 | GYTFTSYW (SEQ ID NO: | IDPSNSET (SEQ ID NO: 252) | ARCDGYYDGLDY (SEQ ID |
| | 157) | | NO: 333) |
| 122 | GYTFTSYW (SEQ ID NO: | INPSNGGT (SEQ ID NO: 231) | ARRIYRTLDY (SEQ ID NO: |
| | 157) | | 335) |
| 123 | GYTFTSYW (SEQ ID NO: | INPSNGGT (SEQ ID NO: 231) | TITGFDV (SEQ ID NO: 535) |
| | 157) | | |
| 124 | GYTFTSYW (SEQ ID NO: | IYPSDSYT (SEQ ID NO: 509) | TRQNYYGSSHWYFDV |
| | 157) | | (SEQ ID NO: 541) |
| 125 | GYTFTSYW (SEQ ID NO: | IDPSDSET (SEQ ID NO: 229) | ANWAWFAY (SEQ ID NO: |
| | 157) | | 317) |
| 126 | GYTFTSYW (SEQ ID NO: | INPSNGRT (SEQ ID NO: 510) | ARDSSGYGAY (SEQ ID NO: |
| | 157) | | 544) |
| 127 | GYTFTSYW (SEQ ID NO: | INPSTGYT (SEQ ID NO: 230) | ARYDGYYYFDY (SEQ ID |
| | 157) | | NO: 545) |
| 128 | GYTFTSYW (SEQ ID NO: | IYPSDSYT (SEQ ID NO: 509) | TSHYYGRAWFAY (SEQ ID |
| | 157) | | NO: 546) |
| 129 | GYTFTSYW (SEQ ID NO: | IDPYDSET (SEQ ID NO: 235) | ARGGRGTWFAY (SEQ ID |
| | 157) | | NO: 553) |
| 130 | GYTFTSYW (SEQ ID NO: | IYPSDSYT (SEQ ID NO: 509) | TRTGGSTMTPWFAY (SEQ |
| | 157) | | ID NO: 556) |
| 131 | GYTFTSYY (SEQ ID NO: 158) | INPSNGGT (SEQ ID NO: 231) | TNGGGWY (SEQ ID NO: |
| | | | 304) |
| 132 | GYTFTSYY (SEQ ID NO: 158) | INPSNSGT (SEQ ID NO: 256) | TRGGDYDASWFAY (SEQ |
| | | | ID NO: 340) |
| 133 | GYTFTSYY (SEQ ID NO: 158) | IYPGDGST (SEQ ID NO: 512) | ARGDGYFAWFAY (SEQ ID |
| | | | NO: 550) |
| 134 | GYTFTTST (SEQ ID NO: 993) | INPRSGYT (SEQ ID NO: | ARHYYFDY (SEQ ID NO: |
| | | 1024) | 612) |
| 135 | GYTFTTYW (SEQ ID NO: | IFPGTGTT (SEQ ID NO: 507) | ARGGYYNSSPFAY (SEQ ID |
| | 483) | | NO: 536) |
| 136 | GYTLTDYS (SEQ ID NO: 152) | INTETGEP (SEQ ID NO: 225) | AWGNHY (SEQ ID NO: 298) |
| 137 | GYTLTDYV (SEQ ID NO: | IYPGSGST (SEQ ID NO: 251) | ARRTARAFDY (SEQ ID NO: |
| | 181) | | 341) |
| 138 | GYTSTAYW (SEQ ID NO: | ITPSTGYT (SEQ ID NO: 1028) | ARGGYFDY (SEQ ID NO: |
| | 994) | | 606) |
| 139 | GYTSTGYW (SEQ ID NO: | INPSTGYT (SEQ ID NO: 230) | ARGGYFDY (SEQ ID NO: |
| | 995) | | 606) |
| 140 | GYTSTNY (SEQ ID NO: 996) | DIHPGGDYSNYNEKFKG | SRNFAY (SEQ ID NO: 1379) |
| | | (SEQ ID NO: 692) | |
| 141 | GYTSTNYW (SEQ ID NO: | IHPGGDYS (SEQ ID NO: | TSRNFAY (SEQ ID NO: |
| | 997) | 1012) | 1419) |

TABLE 7
Light Chain CDRs

| Combination | CDRL1 | CDRL2 | CDRL3 |
|---|---|---|---|
| 1 | DNIYNY (SEQ ID NO: 833) | NAK (SEQ ID NO: 28) | QHHYGSPWT (SEQ ID NO: |
| | | | 1215) |
| 2 | EDIYNR (SEQ ID NO: 924) | GAT (SEQ ID NO: 976) | QQYWSTPPT (SEQ ID NO: |
| | | | 1292) |
| 3 | ENIYFS (SEQ ID NO: 927) | NAN (SEQ ID NO: 1142) | KQAYDVPWT (SEQ ID NO: |
| | | | 1058) |
| 4 | ENIYSY (SEQ ID NO: 930) | NAK (SEQ ID NO: 28) | QHHYDTPYT (SEQ ID NO: |
| | | | 30) |
| 5 | ENIYSY (SEQ ID NO: 930) | NAK (SEQ ID NO: 28) | QHHYGSPYT (SEQ ID NO: |
| | | | 1216) |
| 6 | ENIYSY (SEQ ID NO: 930) | NAK (SEQ ID NO: 28) | QHHYDTPYT (SEQ ID NO: |
| | | | 30) |
| 7 | ENIYSF (SEQ ID NO: 928) | NAK (SEQ ID NO: 28) | QHHYGTPYT (SEQ ID NO: |
| | | | 54) |
| 8 | ENIYSF (SEQ ID NO: 928) | NAK (SEQ ID NO: 28) | QHHYGIPYT (SEQ ID NO: |
| | | | 1214) |
| 9 | ENIYSN (SEQ ID NO: 929) | AAT (SEQ ID NO: 600) | QHFWGTPPT (SEQ ID NO: |
| | | | 1212) |
| 10 | ENIYSN (SEQ ID NO: 929) | AAT (SEQ ID NO: 600) | QHFWGTPWT (SEQ ID NO: |
| | | | 1213) |
| 11 | ENIYSN (SEQ ID NO: 929) | AAT (SEQ ID NO: 600) | QHFWGTPWT (SEQ ID NO: |
| | | | 1213) |
| 12 | ENIYSY (SEQ ID NO: 930) | NAK (SEQ ID NO: 28) | QHHYGSPYT (SEQ ID NO: |
| | | | 1216) |
| 13 | ENIYSY (SEQ ID NO: 930) | NAK (SEQ ID NO: 28) | QHHYGIPYT (SEQ ID NO: |
| | | | 1214) |
| 14 | ENIYSY (SEQ ID NO: 930) | NAK (SEQ ID NO: 28) | QHHYGTPYT (SEQ ID NO: |
| | | | 54) |
| 15 | ENIYSY (SEQ ID NO: 930) | NAK (SEQ ID NO: 28) | QHHYDTPYT (SEQ ID NO: |
| | | | 30) |
| 16 | ENIYSY (SEQ ID NO: 930) | NAK (SEQ ID NO: 28) | QHHYGTPWT (SEQ ID NO: |
| | | | 1217) |
| 17 | ENIYSY (SEQ ID NO: 930) | NAK (SEQ ID NO: 28) | QHHYGTPYT (SEQ ID NO: |
| | | | 54) |
| 18 | ENIYSY (SEQ ID NO: 930) | NAK (SEQ ID NO: 28) | QHHYDTPYT (SEQ ID NO: |
| | | | 30) |
| 19 | ENSYSY (SEQ ID NO: 52) | NAK (SEQ ID NO: 28) | QHHYGTPYT (SEQ ID NO: |
| | | | 54) |
| 20 | ENSYSY (SEQ ID NO: 52) | NAK (SEQ ID NO: 28) | QHHYGTPYT (SEQ ID NO: |
| | | | 54) |
| 21 | ENVATY (SEQ ID NO: 932) | GAS (SEQ ID NO: 975) | GQSYRYPYT (SEQ ID NO: |
| | | | 979) |
| 22 | ENVGTY (SEQ ID NO: 933) | GAS (SEQ ID NO: 975) | GQSYSYPYT (SEQ ID NO: |
| | | | 981) |
| 23 | ENVGTY (SEQ ID NO: 933) | GAS (SEQ ID NO: 975) | GQSYSYPYT (SEQ ID NO: |
| | | | 981) |
| 24 | ENVGTY (SEQ ID NO: 933) | GAS (SEQ ID NO: 975) | GQSYSYPWT (SEQ ID NO: |
| | | | 980) |
| 25 | ESVDNYGISF (SEQ ID NO: | AAS (SEQ ID NO: 599) | QQSKEVPYT (SEQ ID NO: |
| | 934) | | 1267) |
| 26 | ESVDSYGNSF (SEQ ID NO: | LAS (SEQ ID NO: 1063) | QQNNEDPYT (SEQ ID NO: |
| | 935) | | 1266) |
| 27 | ESVDSYGNSF (SEQ ID NO: | RAS (SEQ ID NO: 1346) | QQSNEDPRT (SEQ ID NO: |
| | 935) | | 1269) |
| 28 | KAGQNVGTNVA (SEQ ID | SASYRYSG (SEQ ID NO: | QQYNSYPYT (SEQ ID NO: |
| | NO: 1036) | 1362) | 15) |
| 29 | KASQNVGINVA (SEQ ID | SASYRYSG (SEQ ID NO: | QQYNSYPYT (SEQ ID NO: |
| | NO: 1037) | 1362) | 15) |
| 30 | KASQNVGTNVA (SEQ ID | SASYRYSG (SEQ ID NO: | QQYNSYPYT (SEQ ID NO: |
| | NO: 1038) | 1362) | 15) |
| 31 | KASQNVGTNVA (SEQ ID | SASYRYSG (SEQ ID NO: | QQYNSYPWT (SEQ ID NO: |
| | NO: 1038) | 1362) | 36) |
| 32 | KASQNVGTNVA (SEQ ID | SASYRYSG (SEQ ID NO: | QQYNNSPLT (SEQ ID NO: |
| | NO: 1038) | 1362) | 1282) |
| 33 | KASQSVGTNVA (SEQ ID | SASYRYSG (SEQ ID NO: | QQYNNYPWT (SEQ ID NO: |
| | NO: 1040) | 1362) | 1284) |
| 34 | KNSYSY (SEQ ID NO: 1057) | NAK (SEQ ID NO: 28) | QHHYGTPYT (SEQ ID NO: |
| | | | 54) |
| 35 | KSISKY (SEQ ID NO: 1059) | SGS (SEQ ID NO: 1365) | QQHNEYPWT (SEQ ID NO: |
| | | | 1257) |
| 36 | KSLLHSNGNTY (SEQ ID | RMS (SEQ ID NO: 1360) | MQHLEYPYT (SEQ ID NO: |
| | NO: 1060) | | 1136) |
| 37 | KSLLYKDGKTY (SEQ ID | LMS (SEQ ID NO: 1081) | HQLVEYPYT (SEQ ID NO: |
| | NO: 1061) | | 998) |
| 38 | QDIGLN (SEQ ID NO: 1198) | ATS (SEQ ID NO: 618) | LQYASSPFT (SEQ ID NO: |
| | | | 1097) |
| 39 | QDIGSS (SEQ ID NO: 1199) | ATS (SEQ ID NO: 618) | LQYASSPYT (SEQ ID NO: |
| | | | 1101) |
| 40 | QDIGSS (SEQ ID NO: 1199) | ATS (SEQ ID NO: 618) | LQYASSPWT (SEQ ID NO: |
| | | | 1100) |
| 41 | QDIGSS (SEQ ID NO: 1199) | ATS (SEQ ID NO: 618) | LQYASSPRT (SEQ ID NO: |
| | | | 1099) |
| 42 | QDIGSS (SEQ ID NO: 1199) | ATS (SEQ ID NO: 618) | LQYASSPHT (SEQ ID NO: |
| | | | 1098) |
| 43 | QDIGSS (SEQ ID NO: 1199) | ATS (SEQ ID NO: 618) | LQYATFPYT (SEQ ID NO: |
| | | | 1104) |
| 44 | QDINNF (SEQ ID NO: 1200) | RAN (SEQ ID NO: 1345) | LQYDEFPWT (SEQ ID NO: |
| | | | 1105) |
| 45 | QDINRY (SEQ ID NO: 1201) | RAN (SEQ ID NO: 1345) | LQYDEFPYT (SEQ ID NO: |
| | | | 1106) |
| 46 | QDINRY (SEQ ID NO: 1201) | RAN (SEQ ID NO: 1345) | LQYDEFPYT (SEQ ID NO: |
| | | | 1106) |
| 47 | QDIRNY (SEQ ID NO: 1202) | YTS (SEQ ID NO: 1495) | QQGNTLPPT (SEQ ID NO: |
| | | | 1253) |
| 48 | QDIRNY (SEQ ID NO: 1202) | YTS (SEQ ID NO: 1495) | QQGNTLPPT (SEQ ID NO: |
| | | | 1253) |
| 49 | QDISNY (SEQ ID NO: 27) | HTS (SEQ ID NO: 1002) | QQSNTLPPT (SEQ ID NO: |
| | | | 1272) |
| 50 | QDISNY (SEQ ID NO: 27) | HTS (SEQ ID NO: 1002) | QQSNSLPPT (SEQ ID NO: |
| | | | 1271) |
| 51 | QDISNY (SEQ ID NO: 27) | YTS (SEQ ID NO: 1495) | QQGNTLPPT (SEQ ID NO: |
| | | | 1253) |
| 52 | QDISNY (SEQ ID NO: 27) | YAS (SEQ ID NO: 1476) | QQGNTLPWT (SEQ ID NO: |
| | | | 1255) |
| 53 | QDISNY (SEQ ID NO: 27) | YTS (SEQ ID NO: 1495) | QQGNTLPPT (SEQ ID NO: |
| | | | 1253) |
| 54 | QDISNY (SEQ ID NO: 27) | HTS (SEQ ID NO: 1002) | QQSNSLPPT (SEQ ID NO: |
| | | | 1271) |
| 55 | QDISNY (SEQ ID NO: 27) | HTS (SEQ ID NO: 1002) | QQSNTLPPT (SEQ ID NO: |
| | | | 1272) |
| 56 | QDISNY (SEQ ID NO: 27) | HTS (SEQ ID NO: 1002) | QQGNTLPPT (SEQ ID NO: |
| | | | 1253) |
| 57 | QDISNY (SEQ ID NO: 27) | FTS (SEQ ID NO: 972) | QQGNTLPRT (SEQ ID NO: |
| | | | 1254) |
| 58 | QDISNY (SEQ ID NO: 27) | YTS (SEQ ID NO: 1495) | QQVYTLPWT (SEQ ID NO: |
| | | | 1273) |
| 59 | QDISNY (SEQ ID NO: 27) | YTS (SEQ ID NO: 1495) | QQGNTLPWT (SEQ ID NO: |
| | | | 1255) |
| 60 | QDISNY (SEQ ID NO: 27) | YTS (SEQ ID NO: 1495) | QQGNTLPPT (SEQ ID NO: |
| | | | 1253) |
| 61 | QDISNY (SEQ ID NO: 27) | HTS (SEQ ID NO: 1002) | QQSNSLPPT (SEQ ID NO: |
| | | | 1271) |
| 62 | QDISNY (SEQ ID NO: 27) | YTS (SEQ ID NO: 1495) | QQGHTLPPT (SEQ ID NO: |
| | | | 1252) |
| 63 | QDISNY (SEQ ID NO: 27) | NAK (SEQ ID NO: 28) | QHHYDTPYT (SEQ ID NO: |
| | | | 30) |
| 64 | QDVGTA (SEQ ID NO: 1204) | WAS (SEQ ID NO: 1450) | QQYSSYPLT (SEQ ID NO: |
| | | | 1291) |
| 65 | QDVITA (SEQ ID NO: 1205) | SAS (SEQ ID NO: 13) | QQHYSTPRT (SEQ ID NO: |
| | | | 1263) |
| 66 | QDVNTA (SEQ ID NO: 1206) | WAS (SEQ ID NO: 1450) | QQHYSSPWT (SEQ ID NO: |
| | | | 1258) |
| 67 | QDVNTA (SEQ ID NO: 1206) | WAS (SEQ ID NO: 1450) | QQHYSSPWT (SEQ ID NO: |
| | | | 1258) |
| 68 | QDVNTA (SEQ ID NO: 1206) | WAS (SEQ ID NO: 1450) | QQHYSSPWT (SEQ ID NO: |
| | | | 1258) |
| 69 | QDVNTA (SEQ ID NO: 1206) | SAS (SEQ ID NO: 13) | QQHFNSPYT (SEQ ID NO: |
| | | | 1256) |
| 70 | QDVNTA (SEQ ID NO: 1206) | WAS (SEQ ID NO: 1450) | QQHYSSPWT (SEQ ID NO: |
| | | | 1258) |
| 71 | QDVNTAVA (SEQ ID NO: | WAS (SEQ ID NO: 1450) | QQHYSSPWT (SEQ ID NO: |
| | 1207) | | 1258) |
| 72 | QDVRTA (SEQ ID NO: 1208) | STS (SEQ ID NO: 1386) | QQYSNYLTF (SEQ ID NO: |
| | | | 1290) |
| 73 | QDVSTA (SEQ ID NO: 1209) | SAS (SEQ ID NO: 13) | QQHYSTPYT (SEQ ID NO: |
| | | | 1265) |
| 74 | QDVSTA (SEQ ID NO: 1209) | SAS (SEQ ID NO: 13) | QQHYSTPWT (SEQ ID NO: |
| | | | 1264) |
| 75 | QDVSTA (SEQ ID NO: 1209) | SAS (SEQ ID NO: 13) | QQHYSTPPWT (SEQ ID NO: |
| | | | 1262) |
| 76 | QDVSTA (SEQ ID NO: 1209) | SAS (SEQ ID NO: 13) | QQHYSTPPT (SEQ ID NO: |
| | | | 1261) |
| 77 | QDVSTA (SEQ ID NO: 1209) | SAS (SEQ ID NO: 13) | QQHYSTPYT (SEQ ID NO: |
| | | | 1265) |
| 78 | QDVSTA (SEQ ID NO: 1209) | SAS (SEQ ID NO: 13) | QQHYSTHVH (SEQ ID NO: |
| | | | 1259) |
| 79 | QDVSTA (SEQ ID NO: 1209) | SAS (SEQ ID NO: 13) | QQHYSTPFT (SEQ ID NO: |
| | | | 1260) |
| 80 | QEISGY (SEQ ID NO: 1210) | AAS (SEQ ID NO: 599) | LQYASYPYT (SEQ ID NO: |
| | | | 1103) |
| 81 | QEISGY (SEQ ID NO: 1210) | AAS (SEQ ID NO: 599) | LQYISYPRT (SEQ ID NO: |
| | | | 1107) |
| 82 | QEISGY (SEQ ID NO: 1210) | AAS (SEQ ID NO: 599) | LQYASYPRT (SEQ ID NO: |
| | | | 1102) |
| 83 | QEISGY (SEQ ID NO: 1210) | AAS (SEQ ID NO: 599) | LQYASYPRT (SEQ ID NO: |
| | | | 1102) |
| 84 | QGISNY (SEQ ID NO: 1211) | YTS (SEQ ID NO: 1495) | QQYSKLPWT (SEQ ID NO: |
| | | | 1289) |
| 85 | QNVGIN (SEQ ID NO: 1243) | SAS (SEQ ID NO: 13) | QQYNSYPYT (SEQ ID NO: |
| | | | 15) |
| 86 | QNVGSN (SEQ ID NO: 1244) | SAS (SEQ ID NO: 13) | QQYDSYPYT (SEQ ID NO: |
| | | | 1280) |
| 87 | QNVGTN (SEQ ID NO: 12) | SAS (SEQ ID NO: 13) | QQYNSYPYT (SEQ ID NO: |
| | | | 15) |
| 88 | QNVGTN (SEQ ID NO: 12) | SAS (SEQ ID NO: 13) | QQYNTYPYT (SEQ ID NO: |
| | | | 1287) |
| 89 | QNVGTN (SEQ ID NO: 12) | SAS (SEQ ID NO: 13) | QQYNSYPYT (SEQ ID NO: |
| | | | 15) |
| 90 | QNVGTN (SEQ ID NO: 12) | SAS (SEQ ID NO: 13) | QQYNSYPWT (SEQ ID NO: |
| | | | 36) |
| 91 | QNVGTN (SEQ ID NO: 12) | SAS (SEQ ID NO: 13) | QQYNNYPWT (SEQ ID NO: |
| | | | 1284) |
| 92 | QNVGTN (SEQ ID NO: 12) | SAS (SEQ ID NO: 13) | QQYNTYPYT (SEQ ID NO: |
| | | | 1287) |
| 93 | QNVGTN (SEQ ID NO: 12) | SAS (SEQ ID NO: 13) | QQYNNHPYT (SEQ ID NO: |
| | | | 1281) |
| 94 | QNVGTN (SEQ ID NO: 12) | SAS (SEQ ID NO: 13) | QQYNNYPLT (SEQ ID NO: |
| | | | 1283) |
| 95 | QNVGTN (SEQ ID NO: 12) | SAS (SEQ ID NO: 13) | QQYNRYPYT (SEQ ID NO: |
| | | | 1285) |
| 96 | QNVGTN (SEQ ID NO: 12) | SAS (SEQ ID NO: 13) | XQYNSYPYT (SEQ ID NO: |
| | | | 1475) |
| 97 | QNVGTN (SEQ ID NO: 12) | SAS (SEQ ID NO: 13) | QQYNNSPLT (SEQ ID NO: |
| | | | 1282) |
| 98 | QNVGTN (SEQ ID NO: 12) | SAS (SEQ ID NO: 13) | QQYNSYPRT (SEQ ID NO: |
| | | | 1286) |
| 99 | QNVGTN (SEQ ID NO: 12) | SAS (SEQ ID NO: 13) | SQSTHVPYT (SEQ ID NO: |
| | | | 1375) |
| 100 | QNVGTN (SEQ ID NO: 12) | SAS (SEQ ID NO: 13) | QQYNSYPRT (SEQ ID NO: |
| | | | 1286) |
| 101 | QNVGTN (SEQ ID NO: 12) | SAS (SEQ ID NO: 13) | QQYNSYPYT (SEQ ID NO: |
| | | | 15) |
| 102 | QNVGTN (SEQ ID NO: 12) | SAS (SEQ ID NO: 13) | QQYNTYPYT (SEQ ID NO: |
| | | | 1287) |
| 103 | QNVGTN (SEQ ID NO: 12) | SAS (SEQ ID NO: 13) | QQYNSYPWT (SEQ ID NO: |
| | | | 36) |
| 104 | QNVGTN (SEQ ID NO: 12) | SAS (SEQ ID NO: 13) | QQYNSYPLT (SEQ ID NO: |
| | | | 42) |
| 105 | QNVGTN (SEQ ID NO: 12) | SAS (SEQ ID NO: 13) | QQYNSYPLT (SEQ ID NO: |
| | | | 42) |
| 106 | QNVGTN (SEQ ID NO: 12) | SAS (SEQ ID NO: 13) | QQYNRYPYT (SEQ ID NO: |
| | | | 1285) |
| 107 | QNVGTN (SEQ ID NO: 12) | SAS (SEQ ID NO: 13) | QQYNSYPYT (SEQ ID NO: |
| | | | 15) |
| 108 | QNVGTNVA (SEQ ID NO: | SAS (SEQ ID NO: 13) | QQYNSYPYT (SEQ ID NO: |
| | 1246) | | 15) |
| 109 | QNVRTA (SEQ ID NO: 1247) | MAS (SEQ ID NO: 1122) | LQHWNYPYT (SEQ ID NO: |
| | | | 1093) |
| 110 | QSISDY (SEQ ID NO: 1296) | YAS (SEQ ID NO: 1476) | QNGHSFPRT (SEQ ID NO: |
| | | | 1242) |
| 111 | QSIVHNNGNTY (SEQ ID | KVS (SEQ ID NO: 1062) | FQGSYVPRT (SEQ ID NO: |
| | NO: 1298) | | 971) |
| 112 | QSIVHSNGNTY (SEQ ID NO: | KVS (SEQ ID NO: 1062) | FQGSHVPPT (SEQ ID NO: |
| | 1299) | | 966) |
| 113 | QSIVHSNGNTY (SEQ ID NO: | KVS (SEQ ID NO: 1062) | FQGSHVPRT (SEQ ID NO: |
| | 1299) | | 967) |
| 114 | QSIVHSNGNTY (SEQ ID NO: | KVS (SEQ ID NO: 1062) | FQGSHVPTF (SEQ ID NO: |
| | 1299) | | 968) |
| 115 | QSIVHSNGNTY (SEQ ID NO: | KVS (SEQ ID NO: 1062) | FQGSHVPYT (SEQ ID NO: |
| | 1299) | | 970) |
| 116 | QSLENSNGNTY (SEQ ID | RVS (SEQ ID NO: 1361) | LQVTHVPFA (SEQ ID NO: |
| | NO: 1300) | | 1096) |
| 117 | QSLLDSDGKTY (SEQ ID | LVS (SEQ ID NO: 1116) | WQGTHFPRT (SEQ ID NO: |
| | NO: 1301) | | 1464) |
| 118 | QSLLDSDGKTY (SEQ ID | LVS (SEQ ID NO: 1116) | WQGTHFRT (SEQ ID NO: |
| | NO: 1301) | | 1466) |
| 119 | QSLLDSDGKTY (SEQ ID | LVS (SEQ ID NO: 1116) | WQGTHFPWT (SEQ ID NO: |
| | NO: 1301) | | 1465) |
| 120 | QSLLDSDGKTY (SEQ ID | LVS (SEQ ID NO: 1116) | WQGAHFPWT (SEQ ID NO: |
| | NO: 1301) | | 1461) |
| 121 | QSLLDSDGKTY (SEQ ID | LVS (SEQ ID NO: 1116) | WQGTHFPQT (SEQ ID NO: |
| | NO: 1301) | | 1463) |
| 122 | QSLLDSDGKTY (SEQ ID | LVS (SEQ ID NO: 1116) | WQGTFSSH (SEQ ID NO: |
| | NO: 1301) | | 1462) |
| 123 | QSLLNSSNQKNY (SEQ ID | FAS (SEQ ID NO: 957) | QQHYSTPYT (SEQ ID NO: |
| | NO: 1302) | | 1265) |
| 124 | QSLLYSNGKTY (SEQ ID | QVS (SEQ ID NO: 1344) | LQGTYYPTWT (SEQ ID NO: |
| | NO: 1303) | | 1092) |
| 125 | QSLLYSSNQKNY (SEQ ID | WAS (SEQ ID NO: 1450) | QQYYSYPWT (SEQ ID NO: |
| | NO: 1304) | | 1294) |
| 126 | QSLLYSSNQKNY (SEQ ID | WAS (SEQ ID NO: 1450) | QQYYSYRT (SEQ ID NO: |
| | NO: 1304) | | 1295) |
| 127 | QSLLYSSNQKNY (SEQ ID | WAS (SEQ ID NO: 1450) | QQYYSYP (SEQ ID NO: |
| | NO: 1304) | | 1293) |
| 128 | QSLVHNNGNTY (SEQ ID | KVS (SEQ ID NO: 1062) | SQSTHVPLT (SEQ ID NO: |
| | NO: 1305) | | 1370) |
| 129 | QSLVHSNGNTY (SEQ ID | EVS (SEQ ID NO: 944) | SQSTHVPYT (SEQ ID NO: |
| | NO: 1306) | | 1375) |
| 130 | QSLVHSNGNTY (SEQ ID | KVS (SEQ ID NO: 1062) | SQSTHVPPT (SEQ ID NO: |
| | NO: 1306) | | 1371) |
| 131 | QSLVHSNGNTY (SEQ ID | KVS (SEQ ID NO: 1062) | SQSTHVPWT (SEQ ID NO: |
| | NO: 1306) | | 1374) |
| 132 | QSLVHSNGNTY (SEQ ID | KVS (SEQ ID NO: 1062) | SQSTHVPT (SEQ ID NO: |
| | NO: 1306) | | 1373) |
| 133 | QSLVHSNGNTY (SEQ ID | KVS (SEQ ID NO: 1062) | SQSTHVPRT (SEQ ID NO: |
| | NO: 1306) | | 1372) |
| 134 | QSLVHSNGNTY (SEQ ID | KVS (SEQ ID NO: 1062) | SQSTHVYT (SEQ ID NO: |
| | NO: 1306) | | 1376) |
| 135 | QSLVHSNGNTY (SEQ ID | KVS (SEQ ID NO: 1062) | SQSTHVPLT (SEQ ID NO: |
| | NO: 1306) | | 1370) |
| 136 | QSLVHSYGNTY (SEQ ID | KVS (SEQ ID NO: 1062) | SQSTHVPHT (SEQ ID NO: |
| | NO: 1307) | | 1369) |
| 137 | QSVDYDGDSY (SEQ ID NO: | AAS (SEQ ID NO: 599) | QQSNEDPYT (SEQ ID NO: |
| | 1308) | | 1270) |
| 138 | QSVDYDGDSY (SEQ ID NO: | ATS (SEQ ID NO: 618) | QQSNEDPLT (SEQ ID NO: |
| | 1308) | | 1268) |
| 139 | QSVNND (SEQ ID NO: 1309) | YAS (SEQ ID NO: 1476) | QQAYWSPYT (SEQ ID NO: |
| | | | 1248) |
| 140 | QSVNND (SEQ ID NO: 1309) | YAS (SEQ ID NO: 1476) | QQDYRSPYT (SEQ ID NO: |
| | | | 1249) |
| 141 | QSVNND (SEQ ID NO: 1309) | YAS (SEQ ID NO: 1476) | QQAYWSPYT (SEQ ID NO: |
| | | | 1248) |
| 142 | QSVSND (SEQ ID NO: 1310) | YAS (SEQ ID NO: 1476) | QQDYSSPWT (SEQ ID NO: |
| | | | 1251) |
| 143 | QSVSND (SEQ ID NO: 1310) | YAS (SEQ ID NO: 1476) | QQDYSSPPT (SEQ ID NO: |
| | | | 1250) |
| 144 | QTLLHSDGNTY (SEQ ID | QVS (SEQ ID NO: 1344) | FQGSHVPWT (SEQ ID NO: |
| | NO: 1311) | | 969) |
| 145 | SRVTY (SEQ ID NO: 1380) | DTS (SEQ ID NO: 839) | HQRSGYSYT (SEQ ID NO: |
| | | | 1000) |
| 146 | SSIGY (SEQ ID NO: 1381) | DTS (SEQ ID NO: 839) | HQRGSYPWT (SEQ ID NO: |
| | | | 999) |
| 147 | SSISSSN (SEQ ID NO: 1382) | GTS (SEQ ID NO: 983) | QQWSSYPLT (SEQ ID NO: |
| | | | 1277) |
| 148 | SSVIY (SEQ ID NO: 1383) | DTS (SEQ ID NO: 839) | QQWTSNPPT (SEQ ID NO: |
| | | | 1279) |
| 149 | SSVSSSY (SEQ ID NO: 1384) | STS (SEQ ID NO: 1386) | HQYHRSPPT (SEQ ID NO: |
| | | | 1001) |
| 150 | SSVSY (SEQ ID NO: 1385) | DTS (SEQ ID NO: 839) | QQWSSNPPT (SEQ ID NO: |
| | | | 1274) |
| 151 | SSVSY (SEQ ID NO: 1385) | DTS (SEQ ID NO: 839) | QQWSSNPPYT (SEQ ID NO: |
| | | | 1275) |
| 152 | SSVSY (SEQ ID NO: 1385) | DTS (SEQ ID NO: 839) | QQWSSYPYT (SEQ ID NO: |
| | | | 1278) |
| 153 | SSVSY (SEQ ID NO: 1385) | LTP (SEQ ID NO: 1115) | QQWSSNPYT (SEQ ID NO: |
| | | | 1276) |
| 154 | TDIDDD (SEQ ID NO: 1391) | EGN (SEQ ID NO: 925) | LQSDNMPLT (SEQ ID NO: |
| | | | 1094) |
| 155 | TDIDDD (SEQ ID NO: 1391) | EGN (SEQ ID NO: 925) | LQSDNMPYT (SEQ ID NO: |
| | | | 1095) |

In some embodiments, the CD25 antibody comprises a “V-D-J-region” heavy chain sequence presented in any one of Table 2A, Table 2B, and Table 2C, and a “V-J-region” light chain sequence presented in any one of Table 3A, Table 3B, and Table 3C, humanized versions thereof, or an amino acid sequence comprising at least a 80%, at least a 85%, at least a 90%, or at least a 95% sequence identity thereto. In some embodiments, the antibody comprises the heavy chain variable region sequence from a particular antibody clone so named by its given “ID” (or a humanized version thereof) and the light chain variable region sequence from the same antibody clone (e.g. the light chain from clone identified by the same “ID”) (or a humanized version thereof). Thus, the antibody clone of origin can be identified by the ID shown in Tables 2A-2C or 3A-3C. For example, in such embodiments, the CD25 antibody comprises the heavy chain variable region of antibody clone “AHH03760” as presented in row 1 of Table 2B (or a humanized version thereof) and the light chain variable region of antibody clone “AHH03760” as presented in row 3 of Table 3B (or a humanized version thereof). In other embodiments, the CD25 antibody comprises the heavy chain variable region sequence from a particular antibody clone so named by its given “ID” (or a humanized version thereof) and the light chain variable region sequence from a different antibody clone (e.g. the light chain from clone identified by the same “ID”) (or a humanized version thereof).

In some embodiments, the CD25 antibody comprises a CDRH1, a CDRH2, and a CDRH3 presented in any one of Table 2A, Table 2B, and Table 2C, and a CDRL1, a CDRL2, and a CDRL3 presented in any one of Table 3A, Table 3B, and Table 3C. In such embodiments, the CD25 antibody comprises the CDRH1, CDRH2, and CDRH3 from a particular antibody clone and the CDRL1, CDRL2, and CDRL3 from the same antibody clone. As discussed above, the antibody clone of origin can be identified by the ID shown in Tables 2A-2C or 3A-3C.

In some embodiments, the CD25 antibody comprises a heavy chain variable region presented in Table 4A, humanized versions thereof, or an amino acid sequence comprising at least a 80%, at least a 85%, at least a 90%, or at least a 95% sequence identity thereto. The variable region may include the contiguous HFR1, CDRH1, HFR2, CDRH2, HFR3, CDRH3, HRF4 sequences to form a complete variable region, humanized versions thereof, or an amino acid sequence comprising at least a 80%, at least a 85%, at least a 90%, or at least a 95% sequence identity thereto. In some embodiments, the CD25 antibody comprises a light chain variable region presented in Table 4B, humanized versions thereof, or an amino acid sequence comprising at least a 80%, at least a 85%, at least a 90%, or at least a 95% sequence identity thereto. The variable region may include the contiguous LFR1, CDRL1, LFR2, CDRL2, LFR3, CDRL3, LRF4 sequences to form a complete variable region, humanized versions thereof, or an amino acid sequence comprising at least a 80%, at least a 85%, at least a 90%, or at least a 95% sequence identity thereto. In some embodiments, the CD25 antibody comprises a complete heavy chain variable region as presented in Table 4A and a complete light chain variable region as presented in Table 4B, including humanized versions thereof, and amino acid sequences comprising at least a 80%, at least a 85%, at least a 90%, or at least a 95% sequence identity thereto. In some embodiments, the CD25 antibody comprises the heavy chain variable region sequence from a particular antibody clone and the light chain variable region sequence from the same antibody clone, including humanized versions thereof and amino acid sequences comprising at least a 80%, at least a 85%, at least a 90%, or at least a 95% sequence identity thereto. In some embodiments, the CD25 antibody comprises the heavy chain variable region sequence from a particular antibody clone and the light chain variable region sequence from a different antibody clone, including humanized versions thereof and amino acid sequences comprising at least a 80%, at least a 85%, at least a 90%, or at least a 95% sequence identity thereto. The antibody clone of origin can be identified by the ID shown in Tables 4A and 4B.

In some embodiments, the CD25 antibody comprises a CDRH1, a CDRH2, and a CDRH3 presented in Table 4A, and a CDRL1, a CDRL2, and a CDRL3 presented in Table 4B. In some embodiments, the CD25 antibody comprises the CDRH1, CDRH2, and CDRH3 from a particular antibody clone and the CDRL1, CDRL2, and CDRL3 from the same antibody clone. In other embodiments, the CD25 antibody comprises the CDRH1, CDRH2, and CDRH3 from a particular antibody clone and the CDRL1, CDRL2, and CDRL3 from a different antibody clone. As discussed above, the antibody clone of origin can be identified by the ID shown in Tables 4A and 4B.

In some embodiments, the CD25 antibody comprises a heavy chain variable region presented in Table 5A, including humanized versions thereof and amino acid sequences comprising at least a 80%, at least a 85%, at least a 90%, or at least a 95% sequence identity thereto. The variable region may include the contiguous HFR1, CDRH1, HFR2, CDRH2, HFR3, CDRH3, HRF4 sequences to form a complete variable region, including humanized versions thereof and amino acid sequences comprising at least a 80%, at least a 85%, at least a 90%, or at least a 95% sequence identity thereto. In some embodiments, the CD25 antibody comprises a light chain variable region presented in Table 5B, including humanized versions thereof and amino acid sequences comprising at least a 80%, at least a 85%, at least a 90%, or at least a 95% sequence identity thereto. The variable region may include the contiguous LFR1, CDRL1, LFR2, CDRL2, LFR3, CDRL3, LRF4 sequences to form a complete variable region, including humanized versions thereof and amino acid sequences comprising at least a 80%, at least a 85%, at least a 90%, or at least a 95% sequence identity thereto. In some embodiments, the CD25 antibody comprises a complete heavy chain variable region as presented in Table 5A and a complete light chain variable region as presented in Table 5B, including humanized versions thereof and amino acid sequences comprising at least a 80%, at least a 85%, at least a 90%, or at least a 95% sequence identity thereto. In some embodiments, the CD25 antibody comprises the heavy chain variable region sequence from a particular antibody clone and the light chain variable region sequence from the same antibody clone, including humanized versions thereof and amino acid sequences comprising at least a 80%, at least a 85%, at least a 90%, or at least a 95% sequence identity thereto. In other embodiments, the CD25 antibody comprises the heavy chain variable region sequence from a particular antibody clone and the light chain variable region sequence from a different antibody clone, including humanized versions thereof and amino acid sequences comprising at least a 80%, at least a 85%, at least a 90%, or at least a 95% sequence identity thereto. The antibody clone of origin can be identified by the ID shown in Tables 5A and 5B.

In some embodiments, the CD25 antibody comprises a CDRH1, a CDRH2, and a CDRH3 presented in Table 5A, and a CDRL1, a CDRL2, and a CDRL3 presented in Table 5B. In some embodiments, the CD25 antibody comprises the CDRH1, CDRH2, and CDRH3 from a particular antibody clone and the CDRL1, CDRL2, and CDRL3 from the same antibody clone. In other embodiments, the CD25 antibody comprises the CDRH1, CDRH2, and CDRH3 from a particular antibody clone and the CDRL1, CDRL2, and CDRL3 from a different antibody clone. As discussed above, the antibody clone of origin can be identified by the ID shown in Tables 5A and 5B.

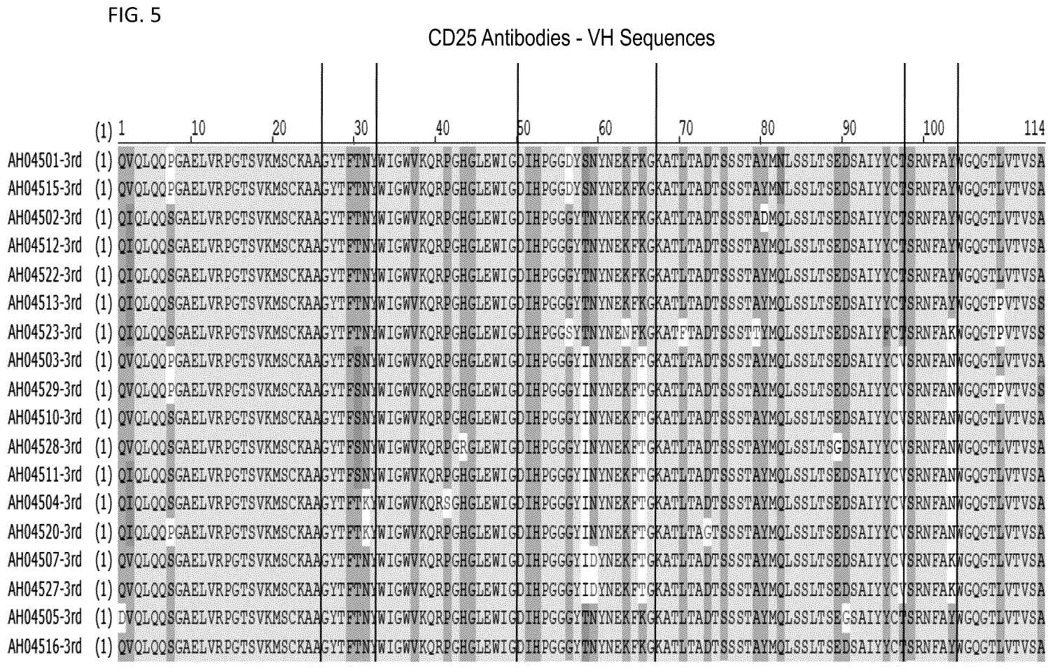

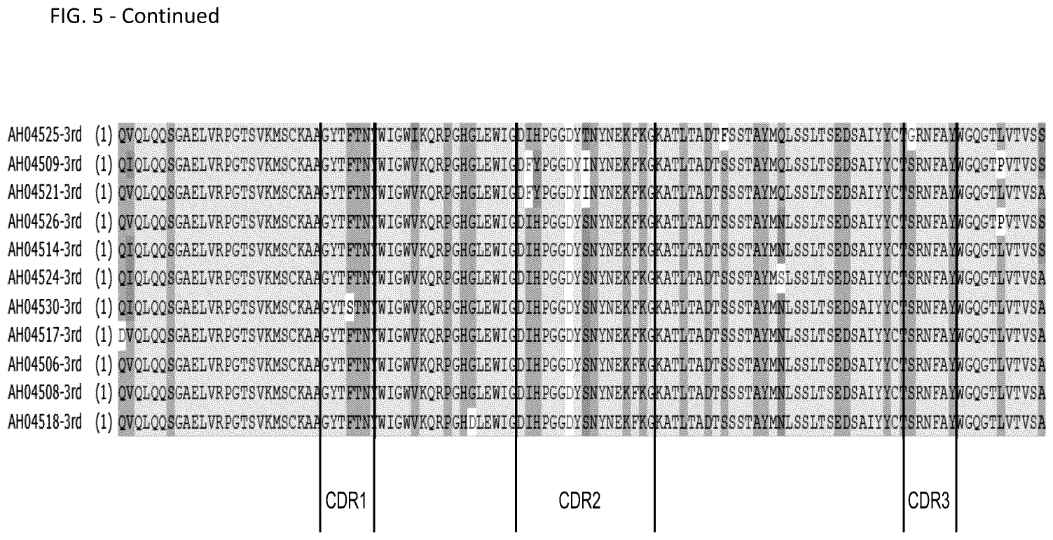

In some embodiments, the CD25 antibody comprises the amino acid sequence of any one of the variable heavy chains presented in Tables 1A, 1C, 1E, 1G, 1I, 1K, 2A, 2B, 2C, 4A, and 5A or FIGS. 3A, 3B and 5, or an amino acid sequence comprising at least a 80%, at least a 85%, at least a 90%, or at least a 95% sequence identity thereto. In some embodiments, the CD25 antibody comprises the amino acid sequence of any one of the variable heavy chains presented in Tables 1A, 1C, 1E, 1G, 1I, 1K, 2A, 2B, 2C, 4A, and 5A or FIGS. 3A, 3B and 5 that has been further humanized.

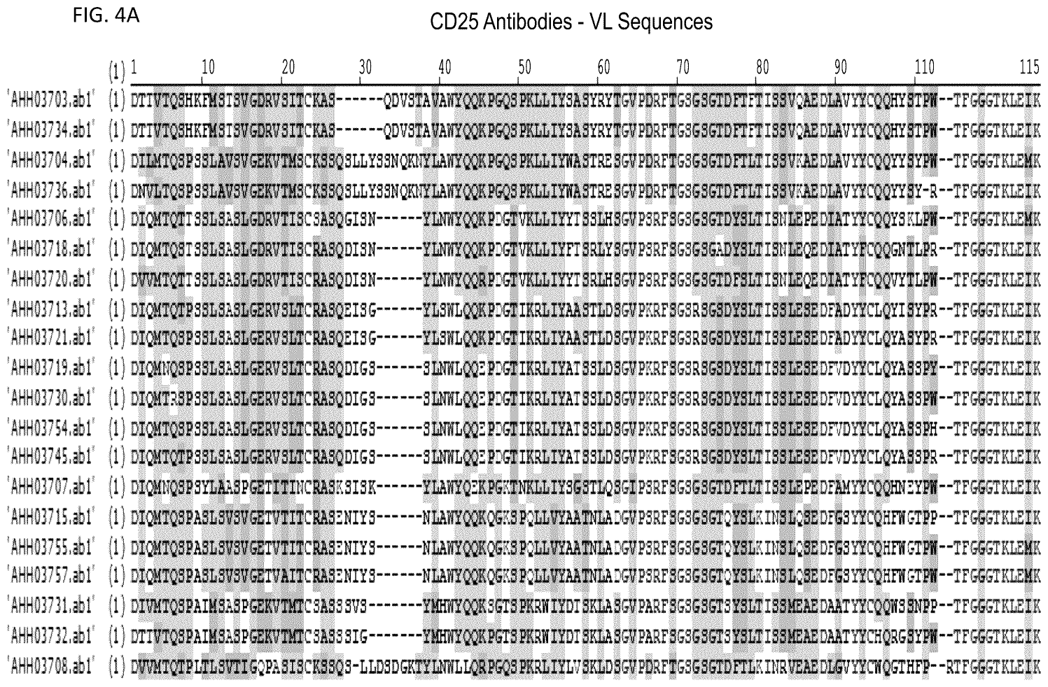

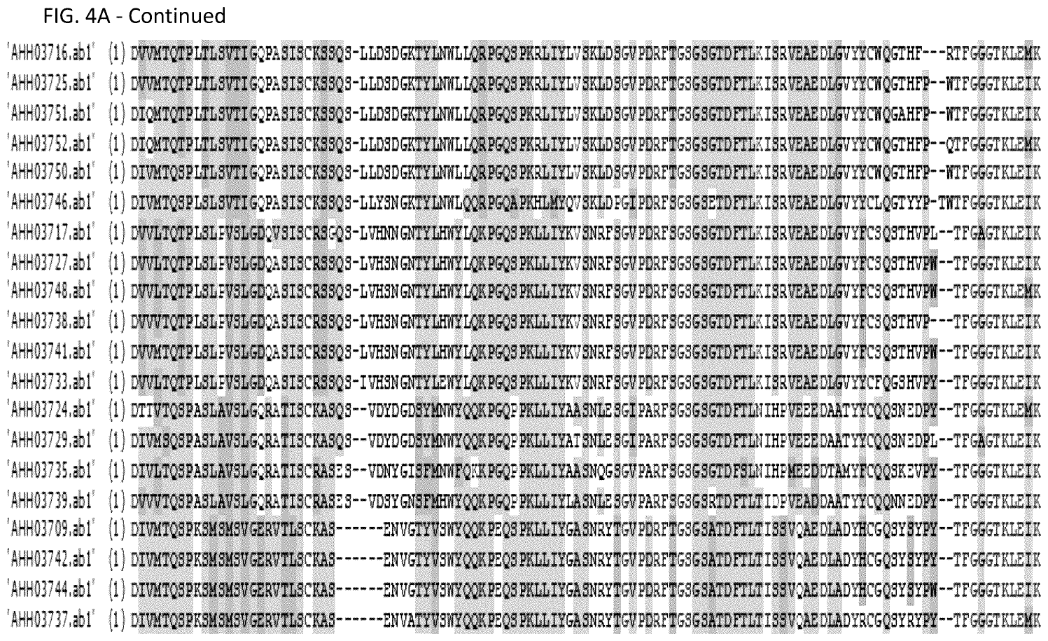

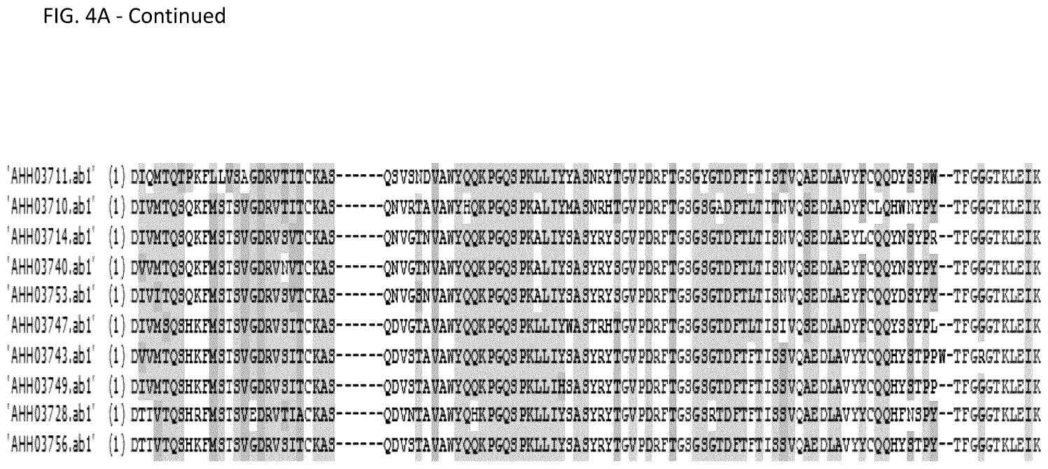

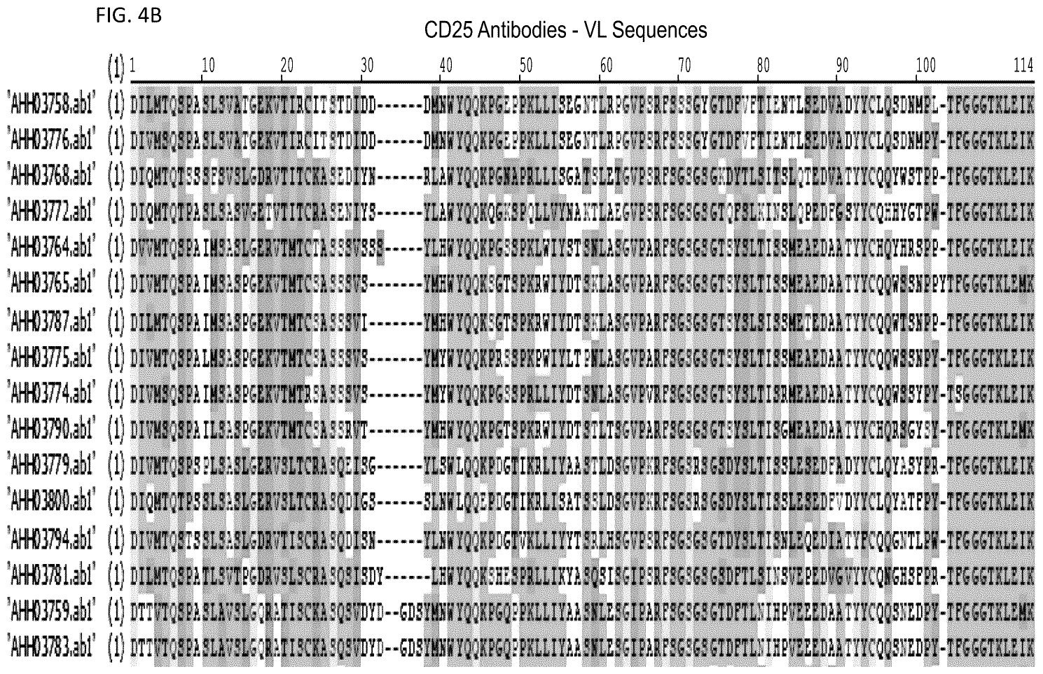

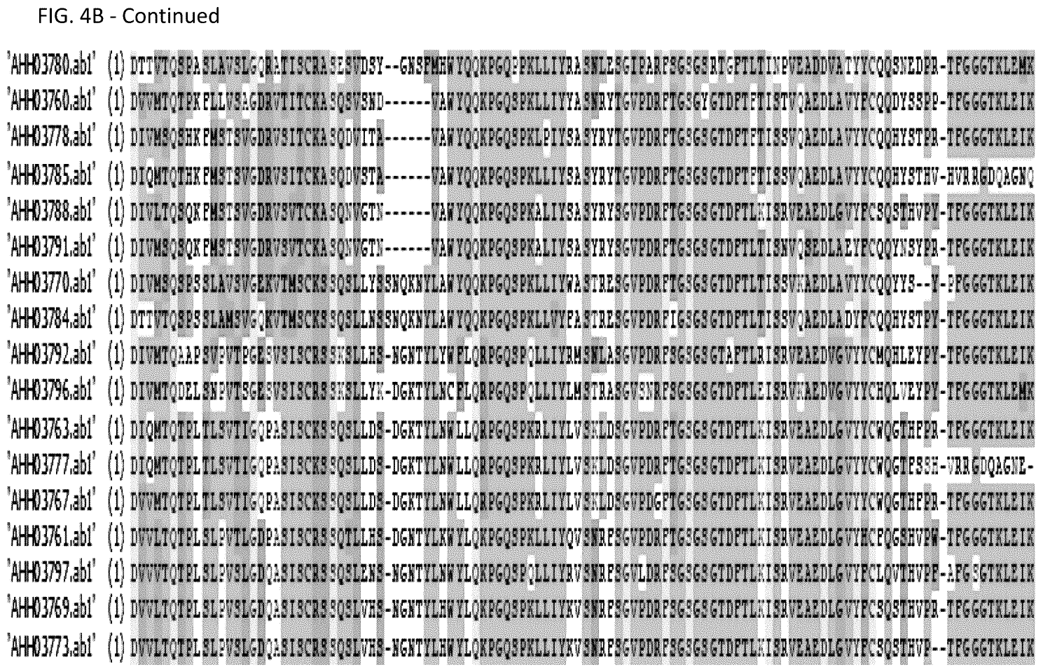

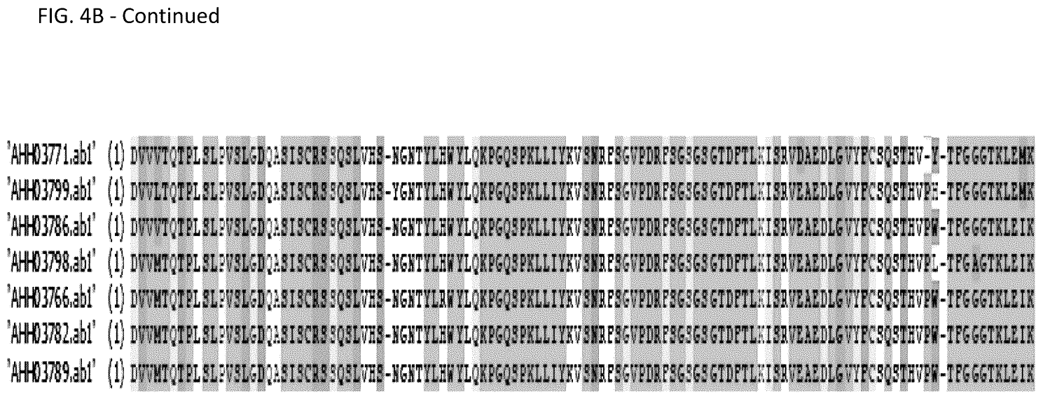

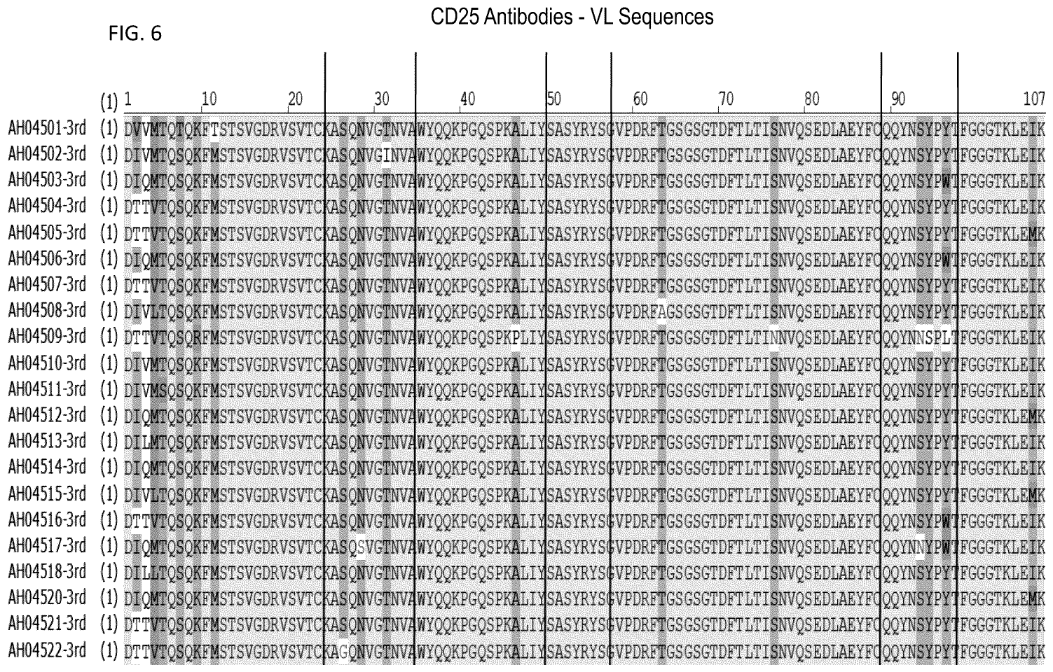

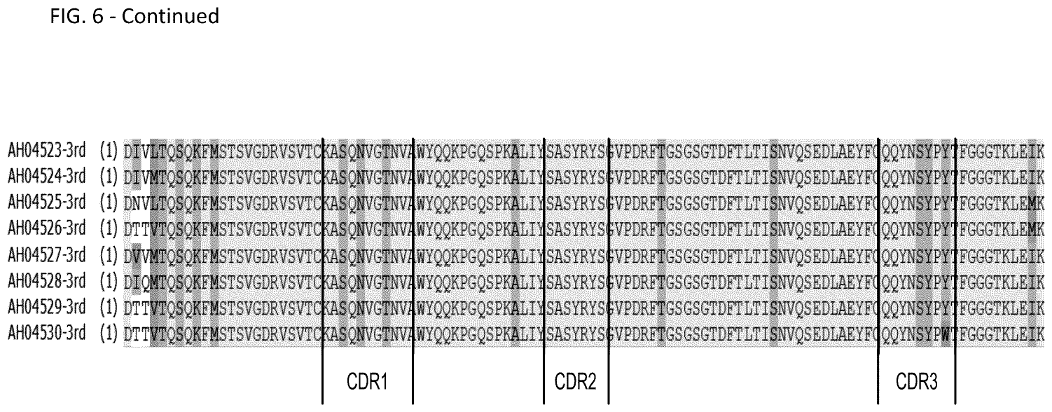

In some embodiments, the CD25 antibody comprises the amino acid sequence of any one of the variable light chains presented in Tables 1B, 1D, 1F, 1H, 1J, 1L, 3A, 3B, 3C, 4B, and 5B or FIGS. 4A, 4B and 6, or an amino acid sequence comprising at least a 80%, at least a 85%, at least a 90%, or at least a 95% sequence identity thereto. In some embodiments, the CD25 antibody comprises the amino acid sequence of any one of the variable heavy chains presented in Tables 1B, 1D, 1F, 1H, 1J, 1L, 3A, 3B, 3C, 4B, and 5B or FIGS. 4A, 4B and 6 that has been further humanized, using conventional techniques

In some embodiments, the CD25 antibody comprises the amino acid sequence of any one of the variable heavy chains presented in Tables 1A, 1C, 1E, 1G, 1I, 1K, 2A, 2B, 2C, 4A, and 5A or FIGS. 3A, 3B and 5, humanized versions thereof, or an amino acid sequence comprising at least a 80%, at least a 85%, at least a 90%, or at least a 95% sequence identity thereto; and the CD25 antibody comprises the amino acid sequence of any one of the variable light chains presented in Tables 1B, 1D, 1F, 1H, 1J, 1L, 3A, 3B, 3C, 4B, and 5B or FIGS. 4A, 4B and 6, humanized versions thereof, or an amino acid sequence comprising at least a 80%, at least a 85%, at least a 90%, or at least a 95% sequence identity thereto.

In some embodiments, VH of the CD25 antibody comprises the amino acid sequence of CDRH1, CDRH2, and CDRH3 as presented in Tables 1A, 1C, 1E, 1G, 1I, 1K, 2A, 2B, 2C, 4A, or 5A, or contained in the sequences presented in FIG. 3A, 3B or 5. In some embodiments, VH of the CD25 antibody comprises the amino acid sequence of CDRH1, CDRH2, and CDRH3 as presented in Table 6.

In some embodiments, VL of the CD25 antibody comprises the amino acid sequence of CDRL1, CDRL2, and CDRL3 as presented in Tables 1B, 1D, 1F, 1H, 1J, 1L, 3A, 3B, 3C, 4B, or 5B, or contained in the sequences presented in FIG. 4A, 4B or 6. In some embodiments, VL of the CD25 antibody comprises the amino acid sequence of CDRH1, CDRH2, and CDRH3 as presented in Table 7.

In some embodiments, VH of the CD25 antibody comprises the amino acid sequence of CDRH1, CDRH2, and CDRH3 as presented in Tables 1A, 1C, 1E, 1G, 1I, 1K, 2A, 2B, 2C, 4A, or 5A, or contained in the sequences presented in FIG. 3A, 3B or 5; and the VL of the CD25 antibody comprises the amino acid sequence of CDRL1, CDRL2, and CDRL3 as presented in Tables 1B, 1D, 1F, 1H, 1J, 1L, 3A, 3B, 3C, 4B, or 5B, or contained in the sequences presented in FIG. 4A, 4B or 6. In some embodiments, VH of the CD25 antibody comprises the amino acid sequence of CDRH1, CDRH2, and CDRH3 as presented in Table 6 and the VL of the CD25 antibody comprises the amino acid sequence of CDRH1, CDRH2, and CDRH3 as presented in Table 7.

In some embodiments, the VH of the antibody comprises the CDR1, CDR2, and CDR3 amino acid sequences presented in Table 5A. In some embodiments, the VL of the antibody comprises the CDR1, CDR2, and CDR3 amino acid sequences presented in Table 5B. In some embodiments, the antibody comprises a VH comprising the CDR1, CDR2, and CDR3 amino acid sequences presented in Table 5A, and a VL comprising the CDR1, CDR2, and CDR3 amino acid sequences presented in Table 5B.

In some embodiments, the antibody comprises the D5 CDR1, CDR2, and CDR3 amino acid sequences, the D5 VH and D5 VL amino acid sequences presented in Tables 1A and 1B, or humanized versions of the D5 VH and D5 VL amino acid sequences presented in Tables 1A and 1B.

In some embodiments, the antibody comprises the D11 CDR1, CDR2, and CDR3 amino acid sequences, the D11 VH and D11 VL amino acid sequences presented in Tables 1C and 1D, or humanized versions of the D11 VH and D11 VL amino acid sequences presented in Tables 1C and 1D.

In some embodiments, the antibody comprises the D16 CDR1, CDR2, and CDR3 amino acid sequences, the D16 VH and D16 VL amino acid sequences presented in Tables 1E and 1F, or humanized versions of the D16 VH and D16 VL amino acid sequences presented in Tables 1E and 1F.

In some embodiments, the antibody comprises the D17 CDR1, CDR2, and CDR3 amino acid sequences, the D17 VH and D17 VL amino acid sequences presented in Tables 1G and 1H, or humanized versions of the D17 VH and D17 VL amino acid sequences presented in Tables 1G and 1H.

In some embodiments, the antibody comprises the D34 CDR1, CDR2, and CDR3 amino acid sequences, the D34 VH and D34 VL amino acid sequences presented in Tables 1I and 1J, or humanized versions of the D34 VH and D34 VL amino acid sequences presented in Tables 1I and 1J.

In some embodiments, the antibody comprises the D36 CDR1, CDR2, and CDR3 amino acid sequences, the D36 VH and D36 VL amino acid sequences presented in Tables 1K and 1L, or the humanized versions of the D36 VH and D36 VL amino acid sequences presented in Tables 1K and 1L.

In some embodiments, the CD25 antibody comprises the CDRH1, CDRH2, and CDRH3 amino acid sequences of any one of the AH04507, AH04522, AH04526, AH04527, AH04734, AH04750, AH05214, AH05247, AH05249, AH05251, AH05256, AH05257, AH05258, AH05259, AH05268, AH05271, AH05274, AH05280, AH05285, AH05286, AH4501, AH4502, AH4503, AH4505, AH4509, AH4511, AH4518, AH4523, AH4524, AH4525, D11, D17, D34, D36, D5, BP003-T2P1C4, BP003-T2P1D10, BP003-T2P1D7, BP003-T2P1E3, or BP003-T2P1D1 clones presented in Table 5A.

In some embodiments, the CD25 antibody comprises the CDRL1, CDRL2, and CDRL3 amino acid sequences of any one of the AH04507, AH04522, AH04526, AH04527, AH04734, AH04750, AH05214, AH05247, AH05249, AH05251, AH05256, AH05257, AH05258, AH05259, AH05268, AH05271, AH05274, AH05280, AH05285, AH05286, AH4501, AH4502, AH4503, AH4505, AH4509, AH4511, AH4518, AH4523, AH4524, AH4525, D11, D17, D34, D36, D5, BP003-T2P1C4, BP003-T2P1D10, BP003-T2P1D7, BP003-T2P1E3, or BP003-T2P1D1 clones presented in Table 5B.

In some embodiments, the CD25 antibody comprises the CDRH1, CDRH2, and CDRH3 amino acid sequences of any one of the AH04507, AH04522, AH04526, AH04527, AH04734, AH04750, AH05214, AH05247, AH05249, AH05251, AH05256, AH05257, AH05258, AH05259, AH05268, AH05271, AH05274, AH05280, AH05285, AH05286, AH4501, AH4502, AH4503, AH4505, AH4509, AH4511, AH4518, AH4523, AH4524, AH4525, D11, D17, D34, D36, D5, BP003-T2P1C4, BP003-T2P1D10, BP003-T2P1D7, BP003-T2P1E3, or BP003-T2P1D1 clones presented in Table 5A; and the CD25 antibody comprises the CDRL1, CDRL2, and CDRL3 amino acid sequences of any one of the AH04507, AH04522, AH04526, AH04527, AH04734, AH04750, AH05214, AH05247, AH05249, AH05251, AH05256, AH05257, AH05258, AH05259, AH05268, AH05271, AH05274, AH05280, AH05285, AH05286, AH4501, AH4502, AH4503, AH4505, AH4509, AH4511, AH4518, AH4523, AH4524, AH4525, D11, D17, D34, D36, D5, BP003-T2P1C4, BP003-T2P1D10, BP003-T2P1D7, BP003-T2P1E3, or BP003-T2P1D1 clones presented in Table 5B.

In some embodiments, the CD25 antibody comprises the CDRH1, CDRH2, CDRH3, CDRL1, CDRL2, AND CDRL3 amino acid sequences of AH04507 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH04522 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH04526 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH04527 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH04734 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH04750 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH05214 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH05247 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH05249 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH05251 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH05256 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH05257 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH05258 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH05259 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH05268 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH05271 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH05274 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH05280 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH05285 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH05286 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH4501 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH4502 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH4503 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH4505 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH4509 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH4511 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH4518 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH4523 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH4524 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of AH4525 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of D11 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of D17 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of D34 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of D36 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of D5 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of BP003-T2P1C4 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of BP003-T2P1D10 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of BP003-T2P1D7 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of BP003-T2P1E3 (presented in Tables 5A and 5B respectively); in some embodiments, the CD25 antibody is a humanized antibody.

In some embodiments, the CD25 antibody comprises the CDRH1, CDRH2, CDRH3, CDRL1, CDRL2, AND CDRL3 amino acid sequences of BP003-T2P1D1 (presented in Tables 5A and 5B respectively); in some embodiments, the CD25 antibody is a humanized antibody.

In some embodiments, the CD25 antibody is conjugated for a variety of purposes including, but not limited to, for use in therapeutics and detection/diagnostics.

Also provided herein are nucleic acid sequences encoding any of the CD25 antibodies provided herein. Exemplary nucleic acid sequences encoding the D5, D11, D16, D17, D34, and D36 VH and VL regions are provided in Tables 1A-1L-one can use humanized versions thereof. Also provided herein are vectors comprising any of the nucleic acids encoding the antibodies, phage comprising such vectors, and host cells comprising such vectors.

### Antibody Generation and Testing

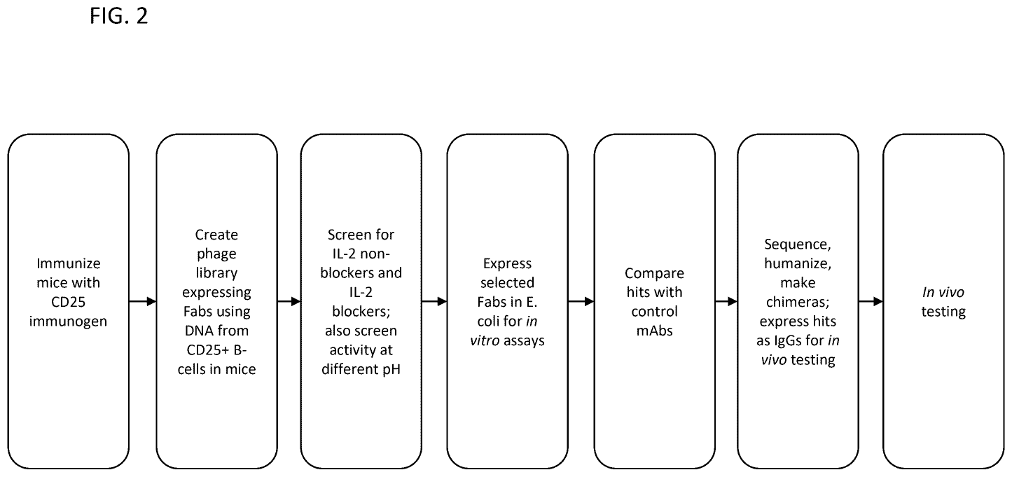

FIG. 2 is an exemplary non-limiting workflow for CD25 antibody discovery, in vitro testing, and in vivo testing, but one of skill in the art recognizes that there are alternative ways to approach antibody discovery and testing.

The CD25 antibodies described herein can be generated by injection of a CD25 complete or partial immunogen into an animal, e.g. a mouse or a rabbit. CD25 immunoge positive B-cells from the animal can be collected, and phage libraries generated therefrom. In some embodiments, the phage express Fab fragments of candidate CD25 antibodies. The phage can undergo multiple rounds of screening (referred to herein as phage panning), for example against successively lower concentrations of a CD25 antigen, to select for those Fab fragments capable of binding CD25 with high affinities. The phage can be screened against CD25 antigen coated beads, or some other substrate, for example. In some embodiments, the screening is carried out a physiological pH (e.g. about pH 7.4). In other embodiments, the screening is carried out at a lower pH, for example, at a pH of about 6.5 to screen for Fab fragments capable of binding the CD25 antigen at a lower pH, for example, for use in a therapeutic context, e.g. for use in a hypoxic, acidic tumor microenvironment.

The CD25 antibodies generated herein may be tested for efficacy using a number of in vitro, in vivo, ex vivo, and/or cell-based assays.

In some embodiments, the CD25 antibodies herein can be assayed for, and further selected based on their ability to deplete regulatory T cells. In particular embodiments, the CD25 antibodies herein can be assayed for, and further selected based on their ability to deplete regulatory T cells in an acidic environment, e.g at a pH lower than physiological pH, e.g. at pH 7.3, 7.2, 7.1, 7.0, 6.9, 6.8, 6.7, 6.6, 6.5, 6.4, 6.3, 6.2, 6.1, or lower.

In some embodiments, the CD25 antibodies herein can be assayed for, and further selected based on a pSTAT5 in vitro assay, to assay for the signaling through the IL-2/IL-2 receptor pathway, the maintenance of which indicates that the antibody is not an IL-2 blocking antibody.

In some embodiments, the CD25 antibodies herein can be assayed for using biosensor screening to characterize molecular interactions.

In some embodiments, the CD25 antibodies herein can be assayed for competition for binding against other known CD25 antibodies, with known mechanisms.

In some embodiments, the CD25 antibodies herein can be assayed for epitope specificity.

In some embodiments, the CD25 antibodies herein can be assayed for their capacity to be a non IL-2 blocker, an IL-2 blocker, or a partial IL-2 blocker.

### Therapeutic Uses

Provided herein are CD25 antibodies for therapeutic use, e.g. for use in proliferative diseases or disorders such as cancer or for use in autoimmune diseases.

Accordingly provided herein are methods of treating a cancer comprising administering to a subject in need thereof a therapeutically effective amount of a therapeutic CD25 antibody. In some embodiments, the cancer is a primary cancer. In some embodiments, the cancer is a metastatic cancer. In some embodiments, the cancer involves a solid tumor; in other embodiments, the cancer involves a liquid tumor, e.g. a blood based cancer. In exemplary embodiments, the CD25 antibody is a non IL-2 blocking antibody.

Accordingly provided herein are methods of treating an autoimmune-related disease or disorder comprising administering to a subject in need thereof a therapeutically effective amount of a therapeutic CD25 antibody. In exemplary embodiments, the CD25 antibody is an IL-2 blocking antibody.

As used herein, a subject refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. Subjects may be male or female.

The administration of any of the therapeutic CD25 antibodies provided herein may be administered in combination with other known drugs/treatments (e.g. small molecule drugs, or biologics. The administration may be sequential or concurrent.

In vivo administration of the therapeutic CD25 antibodies described herein may be carried out intravenously, intratumorally, intracranially, intralesionally (e.g. intralesional injection, direct contact diffusion), intracavitary (intraperitoneal, intralpleural, intrauterine, intrarectal), intraperitoneally, intramuscularly, subcutaneously, topically, orally, transdermally, by implantation, by inhalation, intrathecally, intraventricularly, or intranasally. In an exemplary embodiment, the route of administration is by intravenous injection.

A therapeutically effective amount of the therapeutic antibody will be administered. The appropriate dosage of the therapeutic antibody may be determined based on the severity of the cancer, the clinical condition of the subject, the subject's clinical history and response to the treatment, and the discretion of the attending physician

The dosage amounts of the CD25 antibodies provided herein may vary from about 1 ng/kg up to about 1000 mg/kg of a subject's body weight or more per day, depending upon the route of administration. For repeated administrations over several days or longer, depending on the severity of the cancer, the treatment may be sustained until a desired suppression of symptoms is achieved. Dosage regimens may be useful, depending on the pattern of pharmacokinetic decay that the physician wishes to achieve. For example, dosing an individual from one to twenty-one times a week is provided herein. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is may be monitored by conventional techniques and assays. The dosing regimen may vary over time independently of the dose used.

### Diagnostic Uses

The CD25 antibodies provided herein may be used for diagnostic and detection purposes. Depending on the application, the CD25 antibody may be detected and quantified in vivo or in vitro.

The CD25 antibodies provided herein are amendable for use in a variety of immunoassays. These immunoassays include, but are not limited to enzyme-linked immunosorbent assay (ELISA), Western blot, radioimmunoassay (RIA), flow cytometry, a radioimmunoassay, an immunofluorescence assay, spectrophotometry, radiography, electrophoresis, high performance liquid chromatography (HPLC), or thin layer chromatography (TLC).

The CD25 antibodies provided herein may be comprise a detectable label, for example detectable by spectroscopic, photochemical, biochemical, immunochemical, fluorescent, electrical, optical or chemical methods. Useful labels in the present disclosure include, but are not limited to fluorescent dyes, radiolabels, enzymes, colorimetric lables, avidin or biotin.

In some embodiments, the CD25 antibody is radiolabeled with an isotope, useful for imaging by nuclear medicine equipment (SPECT, PET, or scintigraphy).

### Pharmaceutical Compositions

The disclosure provides compositions comprising therapeutic CD25 antibodies, In some embodiments the composition is sterile. The pharmaceutical compositions generally comprise an effective amount of the therapeutic antibody in a pharmaceutically acceptable excipient.

### Kits and Articles of Manufacture

The disclosure also provides for kits comprising any of the CD25 antibodies described herein, e.g. for either therapeutic or diagnostic uses. In some embodiments, the kits further contain a component selected from any of secondary antibodies, reagents for immunohistochemistry analysis, pharmaceutically acceptable excipient and instruction manual and any combination thereof. In some embodiments, the kit comprises any one or more of the therapeutic compositions described herein, with one or more pharmaceutically acceptable excipients.

The present application also provides articles of manufacture comprising any one of the therapeutic or diagnostic compositions or kits described herein. Examples of an article of manufacture include vials (e.g. sealed vials).

The description provided herein sets forth numerous exemplary configurations, methods, parameters, and the like. It should be recognized, however, that such description is not intended as a limitation on the scope of the present disclosure, but is instead provided as a description of exemplary embodiments.

The following examples are included for illustrative purposes and are not intend to limit the scope of the invention.

### Enumerated Embodiments

Embodiment 1. A monoclonal CD25 antibody which binds to human CD25, and possesses at least one of the following characteristics:
a. the antibody does not disrupt the binding of the IL-2 ligand to the alpha chain of the IL-2 receptor (CD25), and binds to a different epitope than to which 7G7B6 binds;
b. the antibody does not disrupt the binding of the IL-2 ligand to the alpha chain of the IL-2 receptor (CD25), but does disrupt the trimerization of the beta, gamma, and alpha (CD25) chains of the IL-2 receptor;
c. the antibody disrupts the binding of the IL-2 ligand to the alpha (CD25), beta, and/or gamma chains of the IL-2 receptor, and binds to a different epitope than to which daclizumab or baciliximab bind;
d. the antibody exhibits a higher affinity of binding to CD25 at pH lower than 7.4, when compared to the affinity of binding to CD25 at a pH of 7.4;
e. the antibody comprises the amino acid sequence of any one of the variable heavy chains presented in Tables 1A, 1C, 1E, 1G, 1I, 1K, 2A, 2B, 2C, 4A, and 5A or FIGS. 3A, 3B and 5, humanized versions thereof, or an amino acid sequence comprising at least a 80%, at least a 85%, at least a 90%, or at least a 95% sequence identity thereto;
f. the antibody comprises the amino acid sequence of any one of the variable light chains presented in Tables 1B, 1D, 1F, 1H, 1J, 1L, 3A, 3B, 3C, 4B, and 5B or FIGS. 4A, 4B and 6, humanized versions thereof, or an amino acid sequence comprising at least a 80%, at least a 85%, at least a 90%, or at least a 95% sequence identity thereto;
g. the VH of the antibody comprises any one of the amino acid sequence of CDRH1, CDRH2, and CDRH3 as presented in Tables 1A, 1C, 1E, 1G, 1I, 1K, 2A, 2B, 2C, 4A, 5A, and 6 or contained in the sequences presented in FIGS. 3A, 3B and 5;
h. the VL of the CD25 antibody comprises any one of the amino acid sequence of CDRL1, CDRL2, and CDRL3 as presented in Tables 1B, 1D, 1F, 1H, 1J, 1L, 3A, 3B, 3C, 4B, 5B and 7, or contained in the sequences presented in FIGS. 4A, 4B and 6; and
i. the antibody comprises the amino acid sequence of CDRH1, CDRH2, and CDRH3 of any one of the combinations presented in Table 6 and the amino acid sequence of CDRL1, CDRL2, and CDRL3 of any one of the combinations presented in Table 7.

Embodiment 2. The antibody of embodiment 1, wherein the antibody possesses at least two, at least three, at least four, at least five, or at least six of the characteristics provided therein.

Embodiment 3. The antibody of embodiment 1, wherein the antibody does not disrupt the binding of the IL-2 ligand to the alpha chain of the IL-2 receptor (CD25), and binds to a different epitope than to which 7G7B6 binds.

Embodiment 4. The antibody of embodiment 1, wherein the antibody does not disrupt the binding of the IL-2 ligand to the alpha chain of the IL-2 receptor (CD25), but does disrupt the trimerization of the beta, gamma, and alpha (CD25) chains of the IL-2 receptor.

Embodiment 5. The antibody of embodiment 1, wherein the antibody disrupts the binding of the IL-2 ligand to the IL-2 receptor, and binds to a different epitope than to which Daclizumab or Baciliximab bind.

Embodiment 6. The antibody of embodiment 1, wherein the antibody exhibits a higher affinity of binding to CD25 at a pH lower than 7.4, when compared to the affinity of binding to CD25 at a pH of 7.4.

Embodiment 7. The antibody of embodiment 6, wherein the antibody exhibits a higher affinity of binding to CD25 at a pH of about 6.5.

Embodiment 8. The antibody of any one of embodiments 1 to 7, wherein the antibody comprises the amino acid sequence of any one of the variable heavy chains presented in Tables 1A, 1C, 1E, 1G, 1I, 1K, 2A, 2B, 2C, 4A, and 5A or FIGS. 3A, 3B and 5, humanized versions thereof, or an amino acid sequence comprising at least a 80%, at least a 85%, at least a 90%, or at least a 95% sequence identity thereto.

Embodiment 9. The antibody of any one of embodiments 1 to 8, wherein the antibody comprises the amino acid sequence of any one of the variable light chains presented in Tables 1B, 1D, 1F, 1H, 1J, 1L, 3A, 3B, 3C, 4B, and 5B or FIGS. 4A, 4B and 6, humanized versions thereof, or an amino acid sequence comprising at least a 80%, at least a 85%, at least a 90%, or at least a 95% sequence identity thereto.

Embodiment 10. The antibody of any one of embodiments 1 to 7, wherein the VH of the antibody comprises the amino acid sequence of CDRH1, CDRH2, and CDRH3 as presented in Tables 1A, 1C, 1E, 1G, 1I, 1K, 2A, 2B, 2C, 4A, or 5A, or contained in the sequences presented in FIG. 3A, 3B or 5.

Embodiment 11. The antibody of any one of embodiments 1 to 7 and embodiment 10, wherein the VL of the CD25 antibody comprises the amino acid sequence of CDRL1, CDRL2, and CDRL3 as presented in Tables 1B, 1D, 1F, 1H, 1J, 1L, 3A, 3B, 3C, 4B, or 5B, or contained in the sequences presented in FIG. 4A, 4B or 6.

Embodiment 12. The antibody of any one of embodiments 1 to 10, wherein the CD25 antibody comprises the amino acid sequence of CDRH1, CDRH2, and CDRH3 of any one of the combinations presented in Table 6.

Embodiment 13. The antibody of any one of embodiments 1 to 10, wherein the CD25 antibody comprises the amino acid sequence of CDRL1, CDRL2, and CDRL3 of any one of the combinations presented in Table 7.

Embodiment 14. The antibody of any one of embodiments 1 to 13, wherein the antibody is a human antibody.

Embodiment 15. The antibody of any one of embodiments 1 to 13, wherein the antibody is a humanized antibody.

Embodiment 16. The antibody of any one of embodiments 1 to 13, wherein the antibody is a chimeric antibody.

Embodiment 17. The antibody of embodiment 16, wherein the antibody comprises a mouse variable domain, and a human constant domain.

Embodiment 18. The antibody of any one of embodiments 1 to 15, wherein the antibody is an antibody fragment.

Embodiment 19. The antibody of any one of embodiments 1 to 18, wherein the antibody also binds cynomologous monkey CD25.

Embodiment 20. A pharmaceutical composition comprising any one of the antibodies of embodiments 1 to 19.

Embodiment 21. A nucleic acid sequence encoding any one of the antibodies of embodiments 1 to 19.

Embodiment 22. A vector comprising the nucleic acid sequence of embodiment 21.

Embodiment 23. A phage expressing any one of the antibodies of embodiments 1 to 19.

Embodiment 24. A method of treating a subject in need thereof comprising administering to the subject a therapeutically effective amount of any one of the antibodies of embodiments 1 to 19 or the pharmaceutical composition of embodiment 20.

Embodiment 25. A method of depleting the number of regulatory T cells in a subject comprising administering to the subject a therapeutically effective amount of any one of the antibodies of embodiments 1 to 19 or the pharmaceutical composition of embodiment 20.

Embodiment 26. The method of embodiment 24 or 25, wherein the subject suffers from cancer.

Embodiment 27. The method of embodiment 24 or 25, wherein the subject suffers from an autoimmune-related disease or disorder.

Embodiment 28. A method of depleting the number of regulatory T cells in a sample comprising peripheral blood mononuclear cells comprising contacting the sample with any one of the antibodies of embodiments 1 to 19.

Embodiment 29. A kit comprising any one of the antibodies of embodiments 1 to 19 or the pharmaceutical composition of embodiment 20.

### EXAMPLES

### Example 1: Immunization with CD25

Full length CD25 conjugated to KLH was injected into five difference BalbC mice. All injections were done via the tail vein. The immunization protocol was as follows:
Day 0: Immunization 1
Day 14: Immunization 2
Day 28: Immunization 3
Day 35: Bleed

Splenocytes and Leukocytes from four of the five mice were used to generate four phage libraries. The phage libraries contained phage, expressing antigen binding (Fab) fragments. More specifically, a M13 phagemid library using materials from the immunized animals was constructed as follows. (i) Total RNA from leukocytes and splenocytes was extracted; (ii) VH and VL genes were amplified with specific primers; and (iii) VH and VL fragments were inserted into GenScript's M13 phagemid vector by two-step cloning. The library size >2×10\hat{ }8; the insert rate was >90%, and the in-frame rate was >80%. The library was a high diversity library, with at >95% of unique sequences.

### Example 2: Phage Panning for CD25 Binding

3 rounds of phage panning of the Fab-containing phage libraries were carried out, using an ELISA-based assay. The phage were screened against beads coated with CD25. Each round was carried out with a decreasing concentration of CD25. Individual phage clones were expressed in E. Coli TG1 cells (plated on LB/carbenicillin). Single colonies were cultured in 0.4 ml 2YT broth for 30 min at 37° C., and then infected with M13 K07 helper phage for 3 hours at 37° C. Then 50 ug/ml carbenicillin and 10 ug/ml Kanamycin were added to the culture which was then grown overnight at 25° C. The next day, phage was harvested from the cultures.

Positive clones were sequenced as follows. Positive hit clones were cultured overnight and plasmid DNA was prepared with a Qiagen plasmid miniprep kit. Purified plasmids were submitted to Genewiz and ELIM Biopharmaceuticals for sanger sequencing. The VH sequence was obtained with the sequencing primers (SEQ ID NO: 1497)) (ACGCCTGCGAAGTCACCCAT or (AGAAACACAAAGTCTACGCCTGCGAAGTCAC (SEQ ID NO: 1498)). The VL sequence was obtained with the sequencing primer (AGCGGATAACAATTTCACACAGGA (SEQ ID NO: 1499)), or CGGATAACAATTTCACACAG (SEQ ID NO: 1500).

FIGS. 3A, 3B, 4A, and 4B and Tables 2A, 2B, 3A, and 3B show VH and VL sequences of the Fabs selected after the second round of panning.

FIGS. 5 and 6, and Tables 2C and 3C show VH and VL sequences of the Fabs selected after the third round of panning.

Tables 4A, 4B, 5A, and 5B show additional clones after similar panning protocols.

Further pH-based phage panning was carried out.

### Example 3: Phage Panning for CD25 Binding at pH 6.5

Further phage panning was carried out to select for Fab candidates that bind CD25 at lower pH ranges, to select for binders that could bind, for example, in a hypoxic, acidic tumor microenvironment.

The four mouse HuCD25 immunized phage libraries (7807, 7808, 7809, 7810) were transformed by electroporation in TG1 and phage propagated with the addition of CM13 using standard Phage Display protocols (Barbas et al., 2001). TG1 cultures secreting phage were PEG precipitated with PEG/NaCl after incubation on ice for one hour.

The phage libraries (7807, 7808, 7809, 7810) were used for specific pH selections using standard protocols. To deplete antibodies that bind with high affinity at physiological pH, subtractive panning was first carried out by counter-selection of 3×10\hat{ }11 pfu phage (1000-fold representation of a 3×10\hat{ }8) at pH 7.4 by absorption for 1 hour on ELISA plates coated with 10 ug/ml full-length CD25 (400 nM) in PBST pH 7.4. Resulting phage supernatant was collected and pH was adjusted to pH 6.5 with PBST. Subsequent phage panning selections were carried out at pH 6.5

Panning selections were pre-cleared with 25 microliters streptavidin dynabeads with no CD25 antigen after 1 hour incubation. Phage were then added to new pre-blocked Eppendorf LoBind tube. Biotinylated full-length CD25 antigen was added at 100 nM concentration for one hour. Samples were then incubated with 25 microliters streptavidin beads RT for one hour. Samples were pelleted and washed using magnet/magnetic beads 7-9 times with PBST. Tubes were changed twice to remove residual phage.

To elute phage, 800 microliters Glycine pH 2.2 were added to beads and incubated for no more than ten minutes. They were then neutralized with high pH Tris 9.0. Eluted phage were added to 1 ml TG1 freshly grown (OD600˜0.5), and incubated for 20-30 minutes. Fractional log dilution series were plated on plates, and the remainder was transferred to 25 ml 2×YT (2× Yeast Tryptone Broth). These steps were repeated two additional times for a total of three panning rounds, followed by Phage ELISA and Octet screening of periplasmic extracts.

### Sequencing

Positive hit clones were cultured overnight and plasmid DNA was prepared with a Qiagen plasmid miniprep kit. Purified plasmids were submitted to Genewiz and ELIM Biopharmaceuticals for sanger sequencing. The VH sequence was obtained with the sequencing primer (ACGCCTGCGAAGTCACCCAT (SEQ ID NO: 1497)) or (AGAAACACAAAGTCTACGCCTGCGAAGTCAC (SEQ ID NO: 1498)). The VL sequence was obtained with the sequencing primer (AGCGGATAACAATTTCACACAGGA (SEQ ID NO: 1499)) or (CGGATAACAATTTCACACAG (SEQ ID NO: 1500)). VH and VL V-D-J arrangements, nucleic acid alignments, and amino acid alignments were identified by IMGT High V-Quest.

Sequences for selected Fab clones D5, D11, D16, D17, D34, and D36 are presented in Tables 1A-1L.

### Example 4: Phage ELISA protocol and Biosensor/Octet Screening

### ELISA/Extract Preparation

Phage ELISA, for assessing CD25 binding to Fab phage, and periplasmic extract preparation for Fab Octet screening were conducted essentially as described, with modifications noted (Schwimmer et al., 2013).

The CD25 antigen was diluted in PBS, pH 7.4. 50 microliters of antigen solution were made containing 1 ug of CD25 for each well of a 96-well plate to be coated. 50 microliters of antigen solution was added to the ELISA plate wells and incubate overnight at 4° C. Following the incubation, wells were washed twice with PBS, and wells were blocked by adding 200 microliters of 1×PBST 2.0% BSA and incubated for 2 hours at 25° C. Phage were diluted two-fold in 1×PBST 1.0% BSA, pH 6.5. 50 microliters were added per and incubated for 5 minutes at room temperature. The blocking solution was shaken out of the wells, and 50 ul of the dilute phage preparation was added to each well, and incubated for 1 hour at room temperature. The ELISA plate wells were washed 3-5 times with 200 microliters PBST pH 6.5. HRP-conjugated anti-M13 antibodies were diluted (Abcam, ab50370) 1:5000 with 1×PBST 1.0% BSA pH 6.5. 50 microliters of diluted secondary antibody conjugate was added to each well, and incubated for 1 hour at room temperature. ELISA plate wells were washed 3-5 times with 200 microliters PBST pH 6.5. The ECL Lumo substrate was prepared (e.g. Supersignal ELISA Pico Chemiluminescent Substrate) as described, into a 1:1 mixture. 50 microliters substrate solution was added to each well, incubated at room temperature for 5 to 60 minutes before reading.

Colonies were inoculated in 0.03-4 ml 2×YT 0.2% Glucose with 0.1 ml overnight culture (1 ml cultures in 96-well plate or 4 ml cultures in 14-ml falcon tubes). They were incubated at 250-700 rpm for 1.5-2 hours at 37° C. until the OD600˜0.5-1.0. Cultures were induced with 50-400 ul IPTG 0.025-0.1M. In some cases, the temperature was reduced to 30° C. with shaking at 250 rpm. They were then incubated overnight. Next day cultures were harvested by pelleting 3400 rcf for 10-15 minutes. The supernatant was discarded.

Cultures were resuspended with 50-75 ul PPB buffer (30 mM Tris-HCl, pH 8.0, 1 mM EDTA, 20% Sucrose) with 1× Halt Protease Inhibitor and incubated on a rocking platform for 15 minutes at room temperature or 4° C. for 10 min. Cultures were resuspended with 150-225 ul of cold ddH20 with 1× Halt Protease Inhibitor and incubated on a rocking platform for one hour at room temperature or 4° C. for 1-2 hours. The lysate suspension was spun at 15000 rcf for 10-15 min at 4C. Supernatant was collected and diluted.

### Fab Expression and Purification Protocol

Single E. coli colonies were inoculated and grown in 50 2×YT 0.2% Glucose with 0.03-0.5 ml overnight culture in plates or 50 mL cultures. Cultures were incubated at 250-700 rpm at 37° C. for 1.5-hours. Cultures were induced with 50 ul of 25 mM-1M IPTG. The temperature was reduced to 30° C. and rpm to 150. Incubation was done overnight. 50 ml cultures or plates were harvested by pelleting 3400 rcf for 15 minutes. The supernatant was discarded. Cell pellets from 50 mL cultures were placed in a −80° C. freezer for 1 hour, while cultures grown in plates had 75 uL of PPB added with 1× Halt protease inhibitor, EDTA-free (Thermo Fisher Scientific) and vortexed. Plates were shaken at 4° C. for 10 minutes at 1000 rpm. The volume of 225 uL of cold water with 1× Halt protease inhibitor, EDTA-free (Thermo Fisher Scientific) was added to each well. Samples were mixed and shaken at 4° C. for 1-2 hours at max speed i.e. 1000 rpm. Plates were spun at 3500 rpm for 10 mins at 4° C. The supernatant (PPE) was transferred to fresh plates and stored at −20° C. Cell pellets from the 50 mL cultures were removed from the freezer and 5 ml PBS, 10 mM Imidazole was added with 2.5 mg/ml lysozyme and 1× Halt protease inhibitor, EDTA-free (Thermo Fisher Scientific). These were incubated at room temperature for 30 minutes once pellet had fully thawed/mixed. The lysate was centrifuged for 15 minutes at 3400 rcf. The supernatant was removed and pellet discarded. 500 ul Ni-NTA resin was added (pre-washed and pelleted) or a Ni-NTA spin column was used for Fab purification. Incubate with cleared lysate for 30 min-1 hr. This was spun at 1500 rcf. These were washed 5 times with 1 ml PBS, 10 mM Imidazole. Buffer was discarded after each spin. 1 ml PBS, 200 mM Imidazole were added and mixed, incubated for 30 minutes and spun at 1500 rcf for 15 minutes. The eluted protein was stored at 4° C. or 20° C. after determining protein concentration. Zeba columns were used for desalting/buffer exchange.

### Octet/Biosensor Screening of Fabs

For Octet Koff rate screening in raw supernatants, 50 ul of lysate was used in 384-well Pall ForteBio Octet plates. Data was collected on an Octet RED 384 (MD ForteBio). Briefly, Human CD25 was coupled to AR2G tips (1 ug/ml). For data collection, baseline was assessed in PBST 1% BSA buffer at pH 6.5 for 60 seconds. Tips were then moved to 50 ul lysate pH6.5 adjusted and association measured for 300 seconds. Finally, tips were moved to PBST 1% BSA buffer at pH 6.5. Tips were then regenerated with 200 mM Tris-Glycine, pH 2.5 and neutralization with PBST, 1% BSA, pH 6.5. For data analysis, Double referencing was performed on Octet HT 11.0 software (no CD25 on tip as well as blank reference well), for reference subtraction.

Biosensor assays were carried out to determine whether selected Fabs would block IL-2 binding to CD25.

### Octet/Biosensor Screening of Human IgG1 Antibodies

A subset of selected 40 Fabs were reformatted to contain a human IgG1 (referred to herein as human IgG1 antibodies), and data were collected these. The Fabs selected for reformatting are show in Tables 5A (heavy chains) and Table 5B (light chains). The Koff rates and affinity of the different reformatted IgG1 antibodies were evaluated on a FortéBio® Octet RED384™ bio-layer interferometry instrument. Purified antibodies were covalently immobilized on amine reactive biosensors (Fortebio® AR2G), and excess reactive esters were blocked with ethanolamine. Sensors were then dipped into running buffer as a baseline, transferred to wells containing 300 nM full-length CD25, and then transferred back into running buffer. Association of CD25 and subsequent dissociation was recorded in duplicate. Association of running buffer only (to control for sensor drift), and additional sensors with immobilized IgG from human ND serum (to control for non-specific IgG binding), were run as controls. The observed on and off rates were fit using a 1:1 binding fit model, and the equilibrium binding constant (KD) was calculated.

Epitope binning of reformatted human IgG1 antibodies were done using cross-competition assays performed in the classical sandwich format, which involves collecting a baseline, immobilizing the sample antibody onto the biosensor, capturing the antigen, and then incubating with a competitive analyte. The competitive analyte can bind the captured CD25 only if its binding epitope does not overlap with that of the immobilized antibody. Purified antibodies were covalently immobilized on amine reactive biosensors (Fortebio® AR2G), and excess reactive esters were blocked with ethanolamine. Sensors were dipped into running buffer as a baseline, followed by full-length CD25. Then the sensors were transferred to wells containing a competitive analyte (IL-2, 7G7B6, Basiliximab, Daclizumab) or running buffer as a reference. Sensors were regenerated when necessary by exposing them to 3 cycles of 0.1 M Glycine pH 2.0 for 10 seconds, followed by running buffer for 10 seconds.

### Results

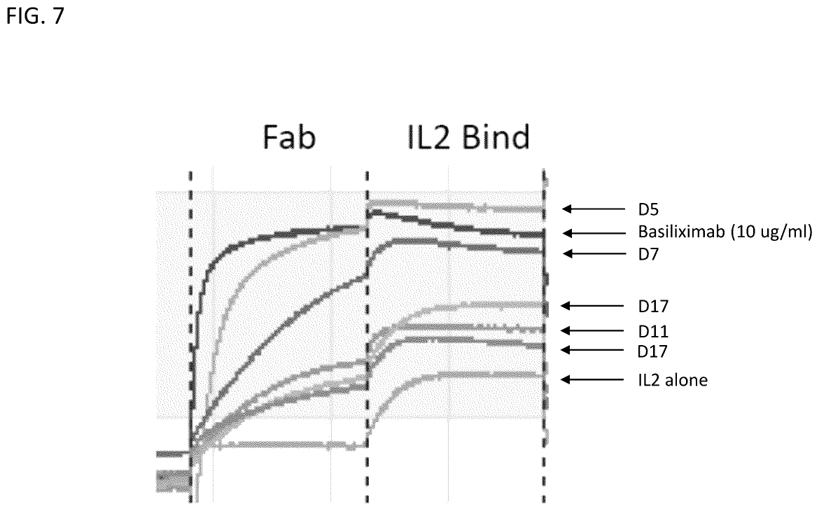

FIG. 7 depicts the identification of non IL-2 blockers and IL-2 blockers (Fabs), using a cross block assay at the molecular level. CD25 was coated on the biosensor tip, and brought into contact with the indicated Fab. IL2 was then added. An increase in signaling shows that the Fab and IL2 have different binding sites on CD25.

Table 8 shows that the Fab clones tested exhibited similar binding kinetics at physiological and acidic pH.

TABLE 8

| pH | Sample ID | kdis (1/s) |
|---|---|---|
| 6.5 | D5 | 4.12E−03 |
| 7.4 | D5 | 3.45E−03 |
| 6.5 | D7 | 3.87E−03 |
| 7.4 | D7 | 2.80E−03 |
| 6.5 | D11 | 5.22E−03 |
| 7.4 | D11 | 4.52E−03 |
| 6.5 | D16 | 5.91E−03 |
| 7.4 | D16 | 4.00E−03 |
| 6.5 | D17 | 5.36E−03 |
| 7.4 | D17 | 3.37E−03 |
| 6.5 | D34 | 2.96E−03 |
| 7.4 | D34 | 2.85E−03 |
| 6.5 | D36 | 1.72E−03 |
| 7.4 | D36 | 8.98E−04 |

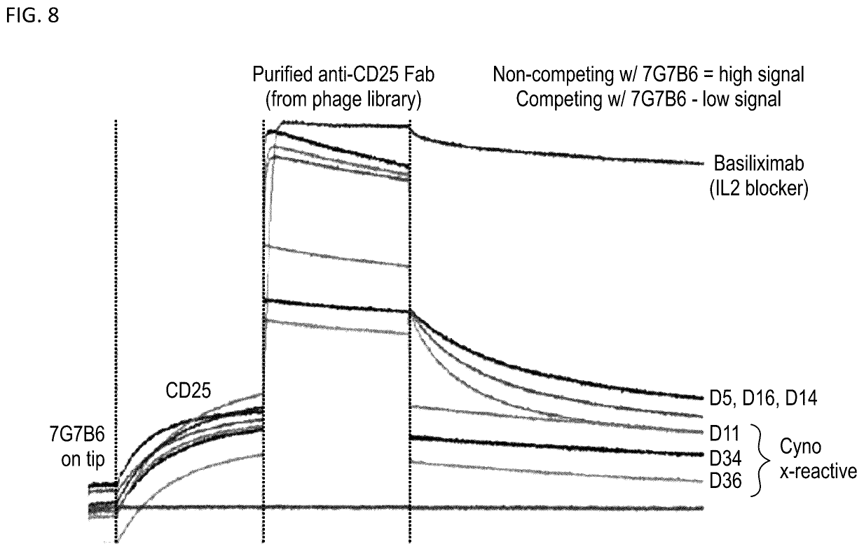

FIG. 8 depicts phage express Fabs of the disclosure that compete with IL-2 non-blocking antibody, 7G7B6. The biosensor tip was coated with the antibody 7G7B6, and brought into contact first with CD25, and then with the indicated Fab or Basiliximab, a known IL2 blocking antibody. Clones D11, D34, and D36 do not bind when 7G7B6 is bound to CD25. Clones D5, D16, and D17 bind, but rapidly come off, indicating a possible cross-blocking function.

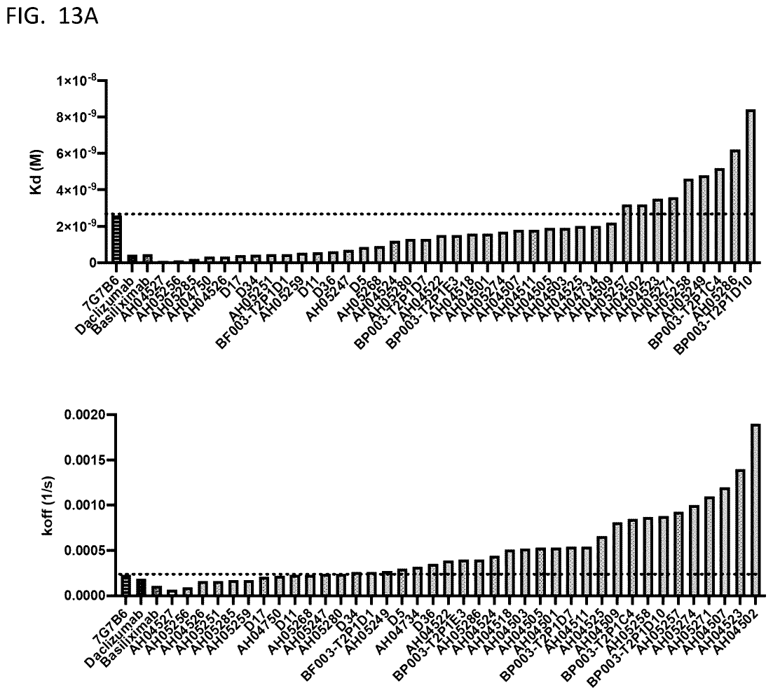

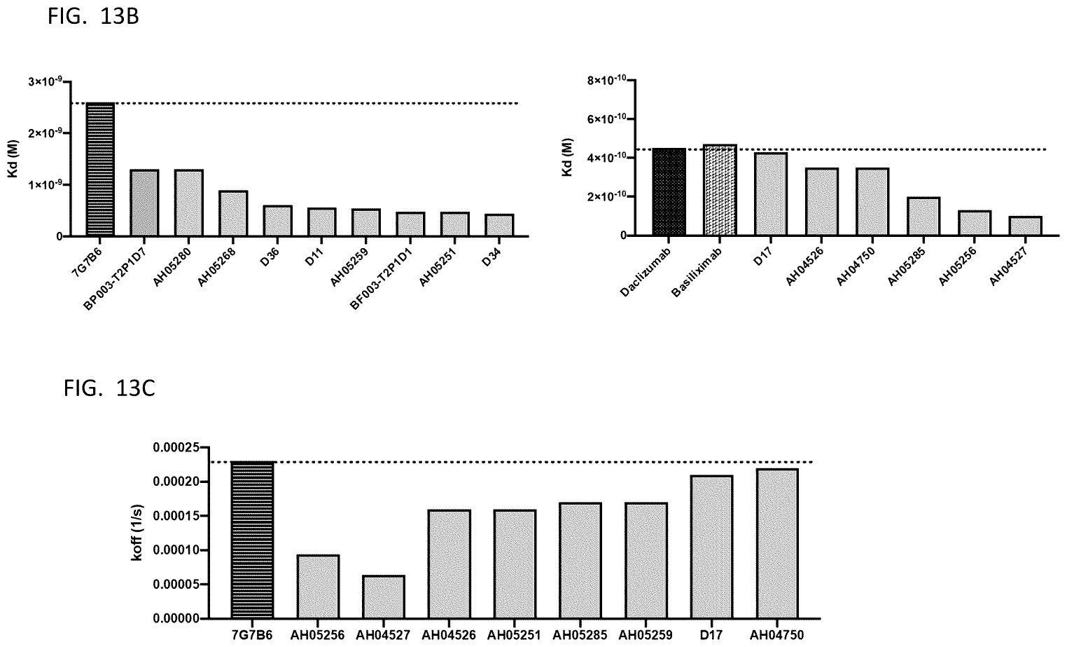

FIGS. 13A-13C shows kinetic analysis for Koff rates and affinity on the reformatted human IgG1 antibody clones (FIG. 13A). KD values of antibodies ranged from 4.4E-10 to 8.4 E-09 with Koff values from 6.4E-05 to 1.9E-03. Several antibodies had higher affinity (FIG. 13B) and Koff values (FIG. 13C) than commercially available antibodies 7G7B6, Daclizumab and Basilixumab (Kd values 2.6E-09, 4.5E-10, 4.7E-10, respectively and Koff rates of 2.3E-04, 1.1E-04, 1.9E-04, respectively). The clones BP003-T2P1D7. AH0580, AH05268, D36, D11, AH05259, BF003-T2P1D1, and D34 have better Kd values than 7G7B6 in this analysis; the clones D17, AH04526, AH04750, AH05285, AH05256, and AH04527 have better Kd values than Daclizumab and Basilixumab in this analysis (FIG. 13B). The clones AH05256, AH04527, AH04526, AH05251, AH05285, AH05259, D17, AH04750 have better Koff rates than 7G7B6 in this analysis (FIG. 13C).

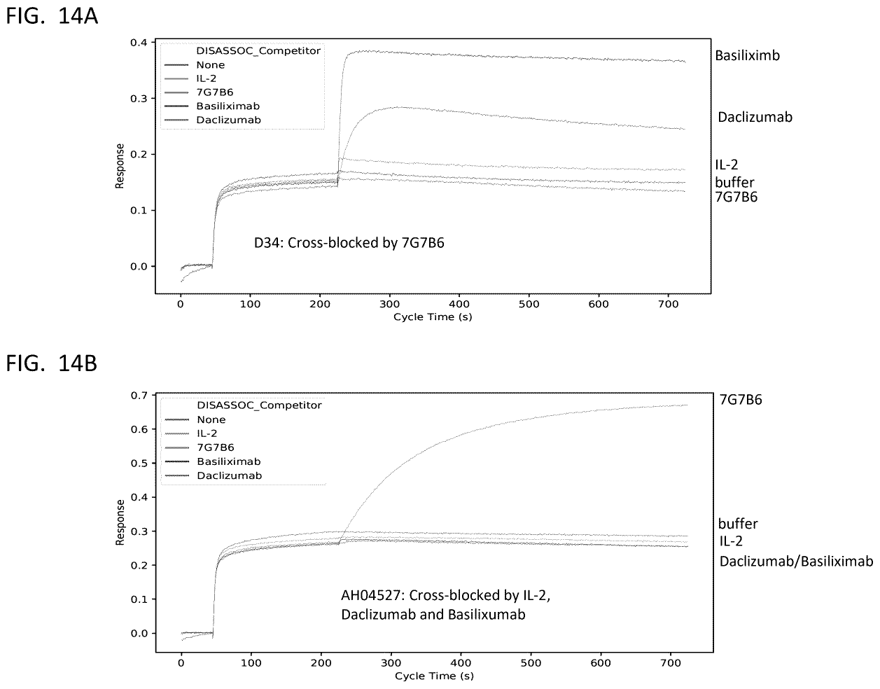

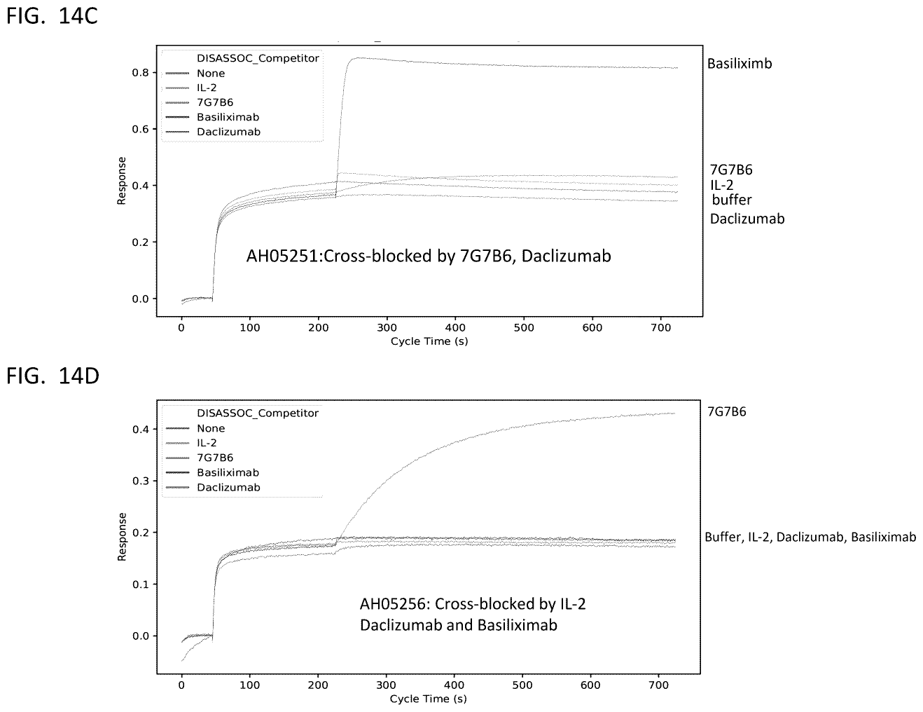

FIGS. 14A-14D Epitope binning was carried out to evaluate the binding of the reformatted human IgG1 Fabs to epitopes overlapping commercially available anti-human CD25 antibodies 7G7B6, Daclizumab and Basilixumab, as well as with IL-2. The blocking profiles of the clones are: (FIG. 14A) blocked by 7G7B6, but not by IL-2, Daclizumab or Basiliximab; (FIG. 14B) blocked by IL-2, Daclizumab, Basiliximab, but not by 7G7B6; (FIG. 14C) blocked by 7G7B6 and Daclizumab, but not by Basilixumab; (FIG. 14D) blocked by IL-2, Daclizumab and Basilixumab. These blocking profiles indicate binding of the antibodies to different epitopes from different approach angles.

Additional epitope mapping of functional epitopes will be performed by Alanine Mutagensis. This method is used as an orthogonal method for binning antibodies as it operates on the functional epitope, rather than the structural epitope defined by competition assays. Various pairs of surface-accessible residues are selected for mutagenesis. Computational modeling is used to confirm that the alanine mutations selected for use in these assays do not impact global or local stability.

### Example 5: Binding Assays

### Cell Binding Assays for CD25 Specific Binding of Human IgG Antibodies

To validate antibody binding to CD25 on cells and for specificity, three cell lines SUDHL-1 and SUDHL-2 (human large diffuse histiocytic lymphoma cell lines, ATCC) and HEK IL-2 reporter cells (Invivogen) were used to test for CD25 binding. SUDHL-1 and HEK IL-2 reporter cell lines are CD25+, while SUDHL-2 are CD25-cells. For each cell line, 100,000 cells were plated in a 96 well round bottom plate in cell buffer (PBS+2% HI FBS) and centrifuged. Antibody concentrations from 1-10 ug/mL were used to test the binding of each antibody. Cell were resuspended in 100 uL of antibodies/well and incubated for 20 minutes on ice. After incubation, cells were centrifuged at 300×g for 5 minutes at room temperature, and resuspended and washed with ice cold cell buffer. Cells were then stained with an anti-human Fc secondary antibody conjugated to AF647 (Biolegend) and incubated on ice for 20 min in the dark. Cells were centrifuged, washed and resuspended in cold cell buffer with DAPI and analyzed using flow cytometry (Cytoflex, Becton Dickinson). DAPI+ cell were excluded from analysis. The mean fluorescence intensity was calculated using the median (FlowJo, TreeStar).

### Recombinant CD25 Cynomolgus Monkey Binding of Human IgG1 Clones

To test for binding to recombinant cynomolgus monkey (cyno) CD25, microtiter plates were coated with 80 ul of 1 ug/ml Cyno CD25 in 50 mM sodium carbonate pH 9.6) at 4° C. overnight. The next day, remove the protein from the wells and block with 200 ul PBS/0.1% BSA/0.05% tween20. The plate was incubated for 1 hour at room temperature. Human IgG1 reformatted Fab clones (human IgG1 antibodies) starting 25 nM and 3 fold serially were diluted in PBT buffer and added to the plate for incubation at RT for 1 hour. The plate was then washed and then 1:2500 dilution of HRP conjugate anti-Fab antibody was added. The plate was washed 5-10 times with PBS/tween20 to remove non-specific binders. TMB perxidase substrate and peroxidase solution were added and incubated for the time needed to develop with 80 uL of ELISA stop solution added at the end. OD measurements were then taken at 450 nM using a plate reader (SpectraMax iD3 Plate Reader, Molecular Devices).

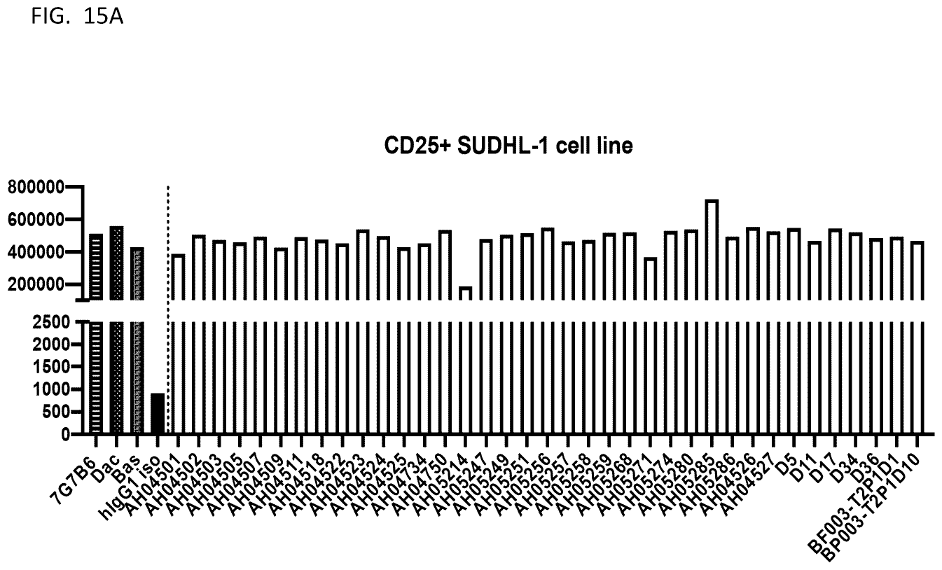

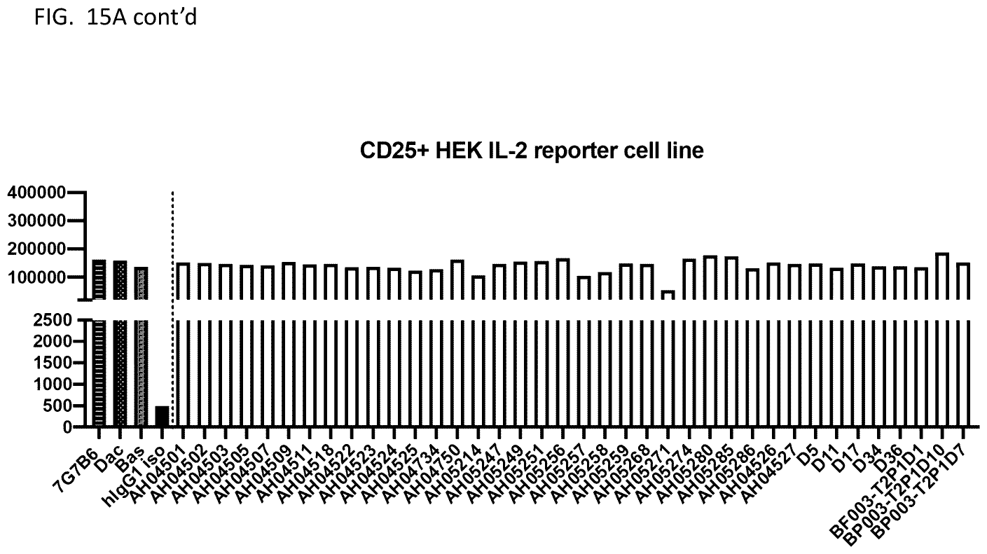

FIGS. 15A-15B shows validation and specific binding of all antibody clones (10 ug/mL) to both CD25+ cells lines, SUDHL-1 and HEK cells, and no binding on CD25-SUDHL-2 cell line. Additionally, the majority of antibodies bound similarily to controls, 7G7B6, Daclizumab, Basilixumab.

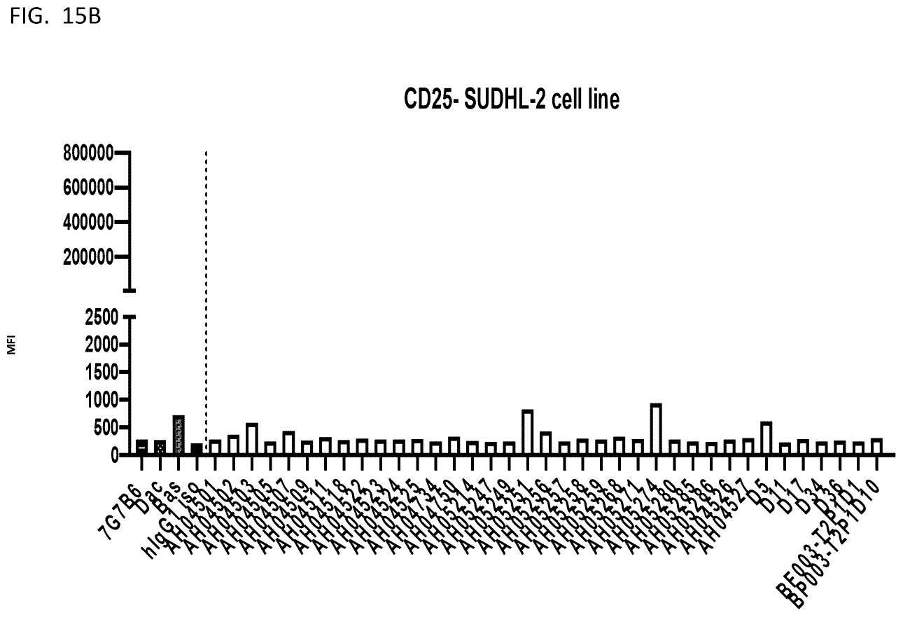

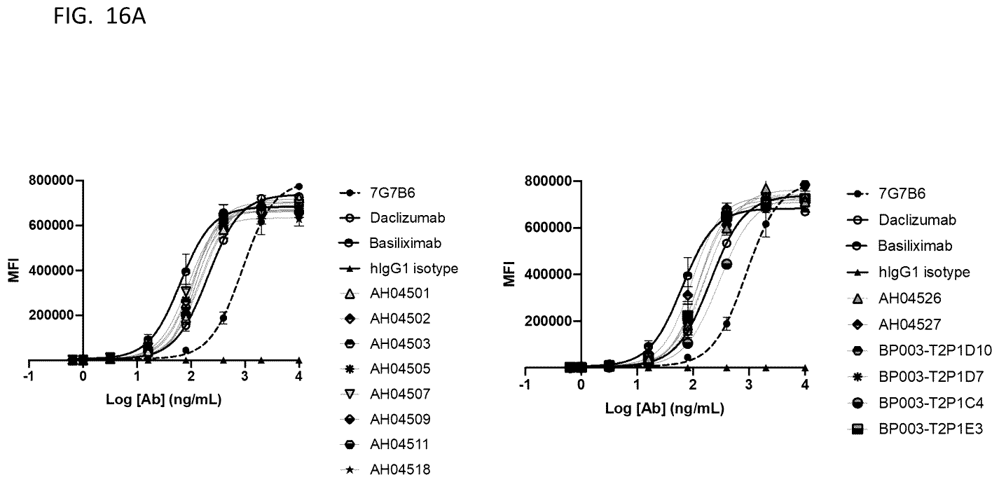

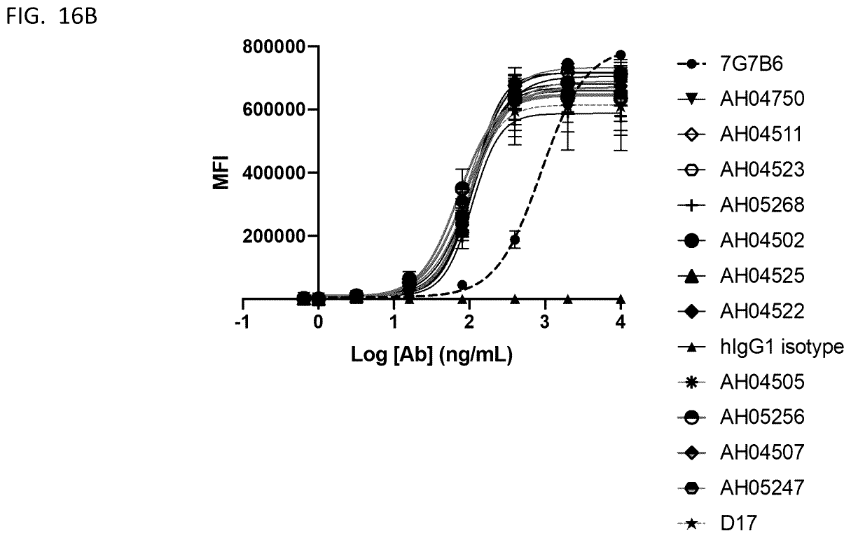

FIGS. 16A-16B show dose response curves of clones starting at 10 ug/mL with 5 fold serial dilutions binding on SUDHL-1 CD25+ cell line (FIG. 16A). A majority of clones bound in a dose dependent manner and had better EC50 values than commercially available 7G7B6 (FIG. 16B, Table 9), clone names referring to those presented in the above tables of the disclosure. Several Clones in Table 9 have better EC50 values than 7G7B6.

TABLE 9

| Clone | EC50 (ng/mL) |
|---|---|
| 7G7B6 | 902.8 |
| Daclizumab | 190.7 |
| Basilixumab | 57.285 |
| AH05256 | 71.48 |
| AH04507 | 88.95 |
| D17 | 95.7 |
| AH04527 | 95.91 |
| AH05247 | 97.73 |
| AH04505 | 105.7 |
| AH04750 | 107.2 |
| AH04511 | 107.6 |
| AH04523 | 108.2 |
| AH05268 | 108.2 |
| AH04502 | 108.9 |
| AH04525 | 109.1 |
| AH04522 | 109.6 |
| AH05257 | 113.3 |
| AH04518 | 114.4 |
| AH04524 | 115.8 |
| AH05259 | 120.7 |
| AH04509 | 121.3 |
| BP003-T2P1D7 | 124.8 |
| BP003-T2P1E3 | 124.9 |
| AH05251 | 132.2 |
| AH04503 | 144.6 |
| AH04501 | 158.9 |
| AH04526 | 164.3 |
| BP003-T2P1D10 | 165.4 |
| AH05258 | 183.9 |
| AH05274 | 227.9 |
| AH05271 | 249.6 |
| BP003-T2P1C4 | 294 |
| AH05280 | 387 |
| D5 | 602.9 |
| AH05286 | 777.5 |
| AH05249 | 896.9 |
| D34 | 1098 |
| D36 | 1491 |
| AH04734 | 1906 |
| D11 | 2120 |
| AH05214 | ~51090 |

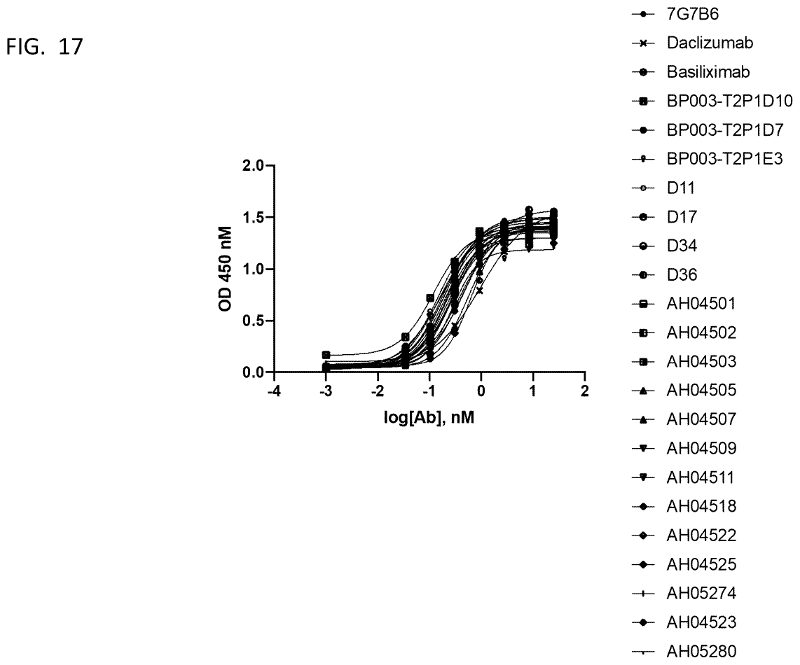

FIG. 17 show dose response curves of several clones that bound to recombinant cyno CD25. Daclizumab and Basiliximab were used as positive controls.

### Example 6: pSTAT5 Assay Using Primary Cells

pSTAT5 Assay for Screening Biological Activity of Fab Clones on Highly Purified Human Regulatory T Cells

The IL-2/JAK3/STAT-5 signaling pathway is involved in the survival and expansion of Tregs. This pathway initiates and maintains the expression of the transcription factor Foxp3, a factor that is essential for the suppressive activity of Tregs. When IL-2 binds to IL-2R, JAK proteins get activated; they are tyrosine kinases that are bound to the cytoplasmic regions of IL-2 receptors. This initiates trans-phosphorylation on specific tyrosine residues generating docking sites for STAT proteins to be recruited and phosphorylated. Dimerized and phosphorylated STATs are then translocated to the nucleus to bind specific DNA sequences, regulating the transcription of several target genes, such as Foxp3 and CD25 in Tregs.

In this example, the functionality of selected Fabs when bound to CD25 on primary human regulatory T cells was assessed. This assay allowed for determination which are IL-2 blockers, non-blockers and partial blockers, as well as potency, based on pSTAT5 levels. Controls used in the assay include a known IL-2 blocker (Daclizumab) and IL-2 non-blocker (7G7B6). First, fresh PBMC cells were isolated from leukocyte reduction system chambers (Stanford Blood Center). Equal volume of cell buffer (PBS+2% HI FBS) was added to each blood sample and added to a Ficoll Paque filled inside a 50 mL SepMate conical tube (Stem Cell Technologies) and followed standard protocol for PBMC isolation using SepMate tubes. To isolate human T regulatory cells from PBMCs, the EasySep Human CD4+CD127low CD25+ Regulatory T cell isolation kit (Stem Cell Technologies) and Regulatory Human CD4+CD25+ T cell kit (Dynabeads) were used. Respective isolation kit protocols were used for magnetic based isolation of cells. Treg isolation was confirmed by staining cells with anti-human CD4, CD25, CD127 antibodies and intracellular Foxp3 staining (BD Biosciences). For the pSTAT5 assay, 100,000 Tregs were plated in a 96 well round bottom plate and centrifuged. Antibody concentrations from 1-5 ug/mL were used. Tregs were resuspended in 50 uL of antibodies/well and incubated for 15 minutes in 37° C. incubator. Next, dilutions for IL-2 were prepared starting at 100 ng/ml concentration with 10 fold dilutions in cell media. IL-2 dilutions were added to the wells at 50 uL/well for 10 minutes in 37° C. incubator. After incubation, the plate was centrifuged at 300×g for 5 minutes at room temperature, and resuspended in 100 uL/well of room temperature fixation buffer (BD Biosciences), for 15 min at room temperature. Cells were spun down and resuspended in cold cell buffer (PBS+2% HI FBS). Cells were then resuspended in 100 uL/well of ice cold permeabilization buffer (BD Biosciences) and incubated on ice for 15 minutes. Cells were washed with cell buffer and resuspended in 50 uL of prepared pSTAT5 antibody for analysis using flow cytometry (Cytoflex, Becton Dickinson).

pSTAT5 Assay for Screening Biological Activity of Antibody Clones Using HEK IL-2 Reporter Cells

In this example, clones were tested using the HEK IL-2 reporter cell line (Invivogen). This cell line was generated by Invivogen to monitor the activation of the JAK-STAT pathway with the binding of IL-2. To obtain a fully active human IL-2 signaling pathway, the reporter cell line was created by stably transfecting HEK293 cells with human IL-2Rα, IL-2Rβ and IL-2Rγ genes, and JAK3 and STAT5 genes. This assay presents a high throughput method for evaluating IL-2 physiology and characterization of IL-2 blockers, non-blockers, partial blockers, with the addition of antibodies and competitors in the presence of IL-2, based on pSTAT5 levels. Controls used in the assay include a known IL-2 blocker (Daclizumab and Basiliximab) and IL-2 non-blocker (7G7B6). For a pSTAT5 assay, 100,000 cells were plated in a 96 well round bottom plate and centrifuged. Antibody concentrations from 1-5 ug/mL) were used. HEK cells were resuspended in 50 uL of antibodies/well and incubated for 15 minutes in 37° C. incubator. Next, dilutions for IL-2 were prepared starting at 10 ng/mL concentration with 10 fold dilutions in cell media. IL-2 dilutions were added to the wells at 50 uL/well for 10 minutes in 37° C. incubator. After incubation, the plate was centrifuged at 300×g for 5 minutes at room temperature, and resuspended in 100 uL/well of room temperature fixation buffer (BD Biosciences), for 15 min at room temperature. Cells were spun down and resuspended in cold cell buffer (PBS+2% HI FBS). Cells were then resuspended in 100 uL/well of ice cold permeabilization buffer (BD Biosciences) and incubated on ice for 15 minutes. Cells were washed with cell buffer and resuspended in 50 uL of prepared pSTAT5 antibody for analysis using flow cytometry (Cytoflex, Becton Dickinson). Percent pSTAT5+ cells were quantified based on the parental population and values were normalized to IL-2 at 10 ng/ml concentration.

### Results

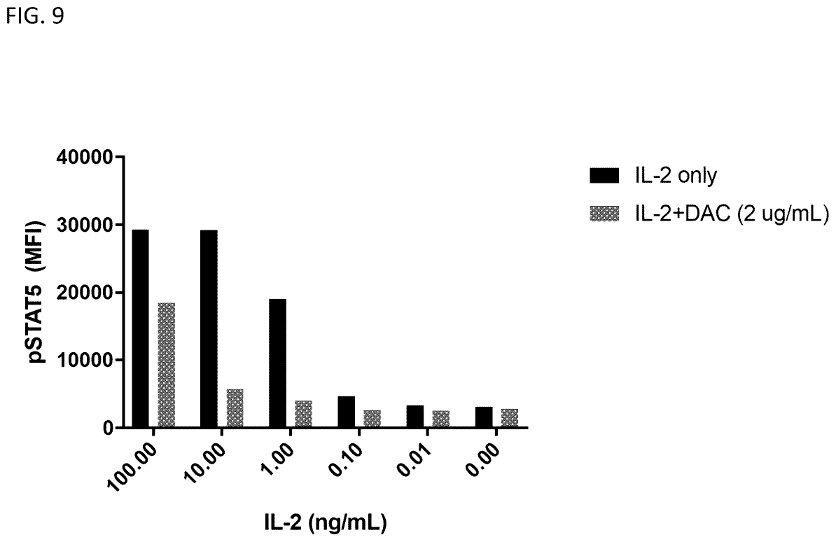

FIG. 9 shows that pSTAT5 signal is IL-2 dose dependent and is inhibited with Daclizumab (IL-2 blocker). pSTAT5 levels were correlated with doses of IL-2, with high IL-2 doses increasing pSTAT5 levels, while lower levels of IL-2 yielded lower levels of pSTAT5. In addition, when Daclizumab, an anti-human IL-2 antibody IL-2 blocker was added at fixed concentration of 2 ug/mL, it inhibited JAK/STAT5 signaling pathway, yielding less pSTAT5 levels.

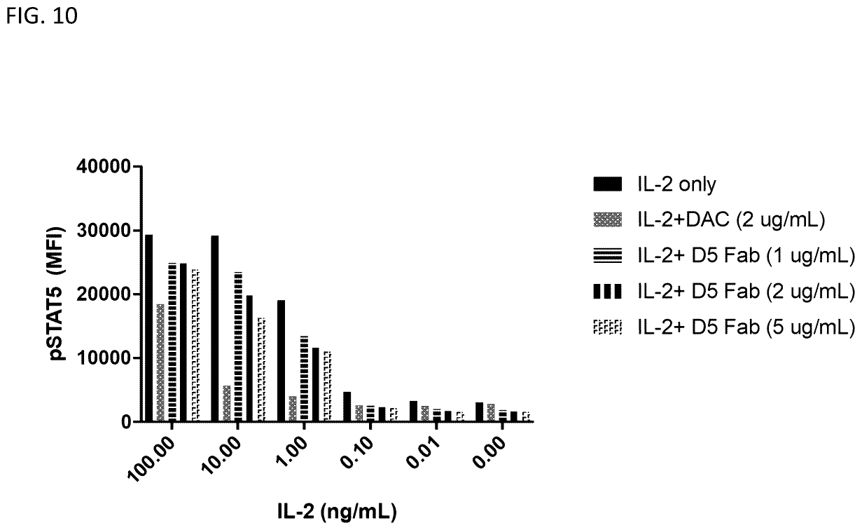

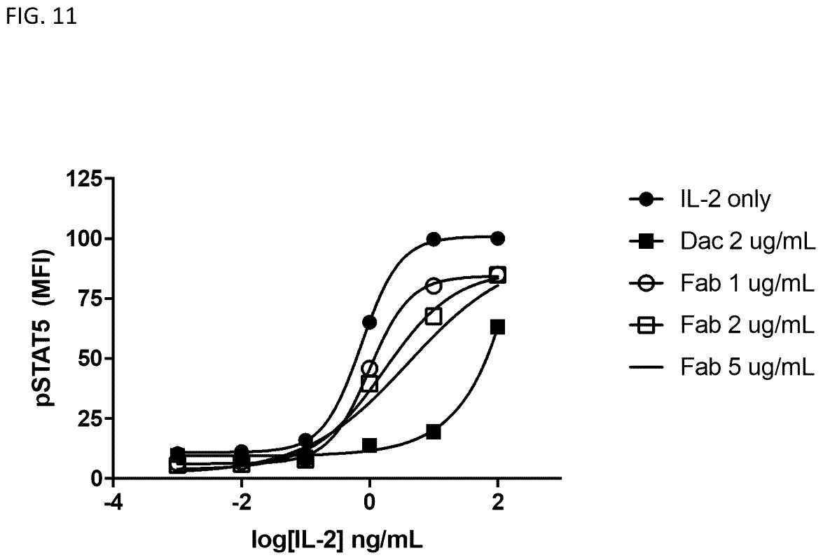

FIG. 10 and FIG. 11 show testing of the D5 Fab in the pSTAT5 assay. The data in FIG. 10 show that the pSTAT5 assay was able to distinguish differences in D5 Fab activity at 1, 2 and 5 ug/mL compared to Daclizumab and IL-2 only. At the different concentrations of D5 there was a dose-dependent decrease in levels of pSTAT5, suggesting that D5 is partially blocking IL-2 binding to IL-2R, even at the lowest concentration of 1 ug/mL. The data is represented by raw MFI levels (mean flurorescence intensity) and by comparing pSTAT5 levels relative to maximum pSTAT5 level generated by the highest dose of IL-2 (100 ng/ml) (FIG. 11).

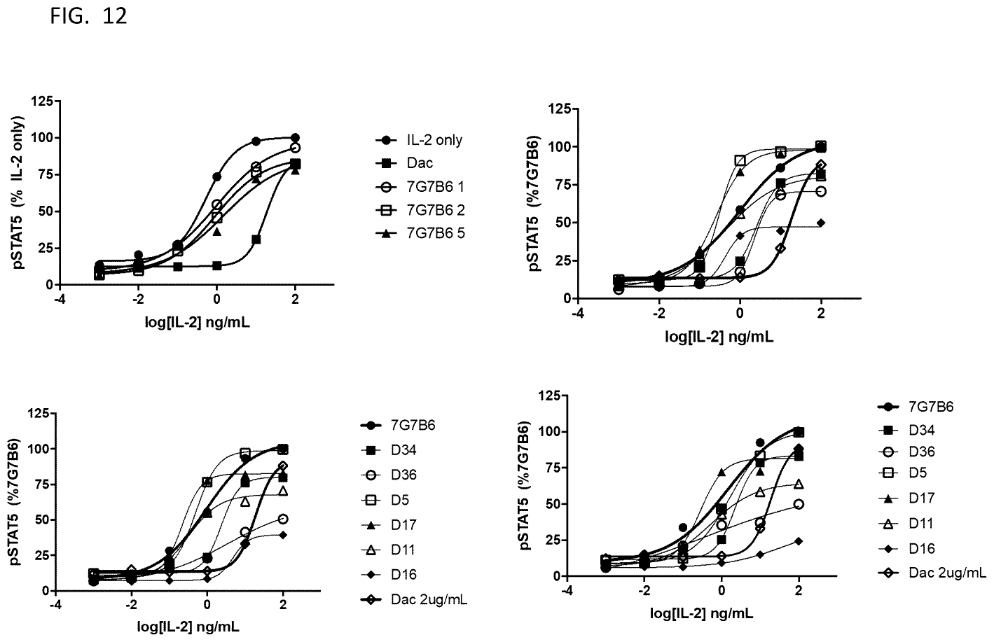

FIG. 12 shows preliminary Fab screen data from one donor. This assay from one donor shows differences in pSTAT5 levels between several Fab clones at 1, 2 and 5 ug/mL compared to the controls (IL-2 only, Daclizumab (Dac, an IL-2 blocker) and 7G7B6 (non IL-2 blocker). Some clones seem to be better IL-2 non-blockers than 7G7B6 while others are better IL-2 blockers than Daclizumab. Complete IL-2 non-blockers would be expected to have similar pSTAT5 levels as IL-2 and would not be expected to be concentration dependent. The data are represented by raw MFI levels (mean fluorescence intensity) and by comparing pSTAT5 levels relative to maximum pSTAT5 level generated by 7G7B6. In addition, the data can be used to observe differences in IC50 potency values of the Fab clones. (Table 10).

TABLE 10

| | IC50 | IC50 | IC50 |
|---|---|---|---|
| | (1 ug/mL) | (2 ug/mL | (5 ug/mL) |
| Ab | [ng/ml] | [ng/ml] | [ng/ml] |
| 7G7B6 | 0.9629 | 0.9058 | 1.47 |
| Dac | | 18.69 | |
| D34 | 2.558 | 2.355 | 2.275 |
| D36 | 2.233 | 3.446 | 0.3782 |
| D5 | 0.3032 | 0.4881 | 1.653 |
| D17 | 0.2511 | 0.2173 | 0.2826 |
| D11 | 0.4137 | 0.3032 | 0.5087 |
| D16 | 0.43 | 4.77 | 40.33 |

Subsequent assays will include the Fab form of Daclizumab and 7G7B6 as well as testing the same Fab clones on multiple donors. In addition, the pSTAT5 assay will be performed at lower pH levels (pH 6.4-6.7) to recapitulate pH of the tumor microenvironment against clone activity at physiological pH.

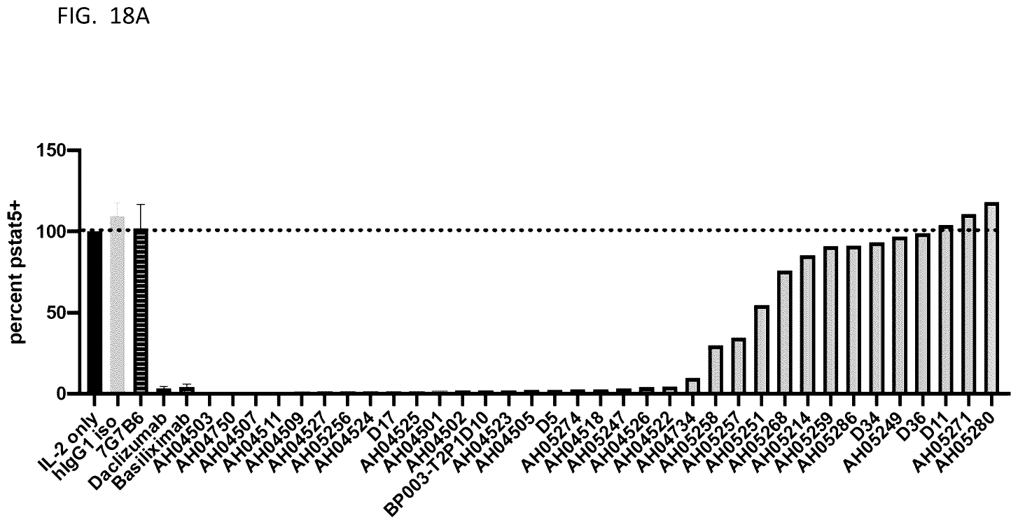

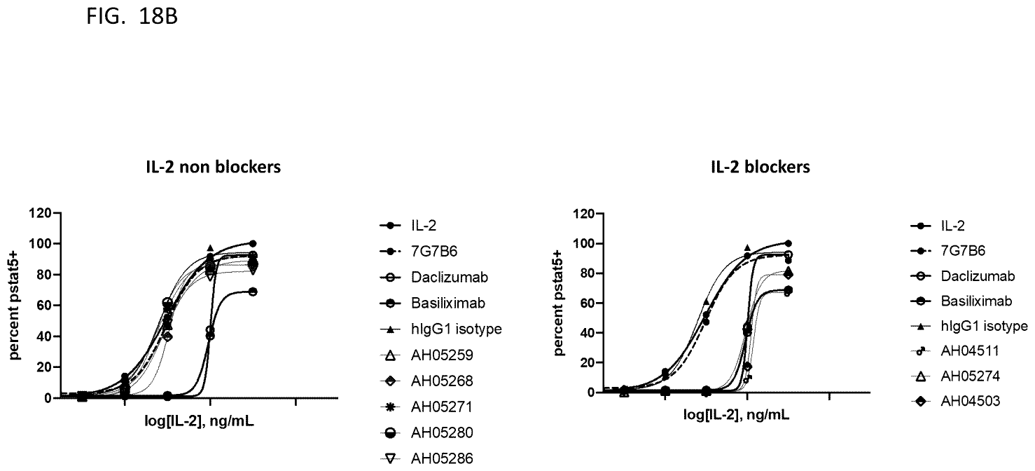

FIGS. 18A-18B show functional characterization of reformatted human IgG1clones (at 5 ug/mL) as IL-2 blockers, non-blockers and partial blockers by measuring pSTAT5 in HEK IL-2 reporter cell lines starting at IL-2 concentration of 10 ng/ml with 10 fold serial dilutions. At IL-2 concentration of 0.1 ng/ml (closer to physiological levels), clones such as AH04503, AH04750 are better IL-2 blockers than either Daclizumab or Basilixumab, while clones AH05280 and AH02571 may be better IL-2 non-blockers than 7G7B6 at 5 ug/mL (A). Clones AH05251, AH05257 are examples of IL-2 partial blockers (FIG. 18A). IL-2 dose response curves clearly depict clones that are IL-2 non-blockers from those that are IL-2 blockers, with curves shifting to the left with 7G7B6 and curves shifting to the right with Daclizumab and Basilixumab, respectively (FIG. 18B).

Continued pSTAT5 studies may be done to test these antibody clones using primary Tregs and multiple donors.

### Example 7: Antibody-Dependent Cell Cytotoxicity (ADCC)

### Functional Cell Killing Assay ADCC and ADCP

One of the mechanisms for Treg depletion is through antibody-dependent cell cytotoxicity (ADCC). This is a cell-mediated immune defense mechanism that usually causes cell death, triggered by the recognition of immune/effector cells to antibodies bound to a specific antigen on a target cell. To elicit ADCC, antibodies with the human Fc subclass, IgG1, is commonly chosen for its effector functions with the ability to bind to the three Fc receptors: FcγRI (CD64), FCyRII (CD32), and FcγRIIIA (CD16), that are expressed on immune cells such as NK cells, monocytes and granulocytes. NK cells predominantly express FcγRIIIA and considered to be the main effector cell in ADCC

In this example, ADCC was quantified using a Lactate dehydrogenase (LDH) Cytotoxicity plate-based colorimetric assay (Thermo Fisher). In this assay, the release of LDH is proportional to the amount of cell killing. Lactate dehydrogenase (LDH) is a cytosolic enzyme, present in all cells, that is released when the plasma membrane is damaged. Extracellular LDH in the media is quantified by a coupled enzymatic reaction in which LDH catalyzes the conversion of lactate to pyruvate via NAD+ reduction to NADH. By addition of diaphorase, NADH is reduced to a tetrazolium salt (INT) to form a formazan product that can be measured at 490 nm. Controls for the assay include the use of Rituximab (anti-CD20 antibody) on human Raji lymphoma cell line and Daclizumab on SUDHL-1 cells (both antibodies are known to induce ADCC). Additional controls include: non-glycosylated human IgG1, as a negative control and non-fucosylated human IgG1, as a positive control. As a first initial screen, reformatted clones were tested in the ADCC assay at one concentration (10 ug/mL) using PBMCs from a single donor. Human PBMCs (ASTARTE Biologics) were thawed the day before and cultured overnight with X-VIVO 15 or 20 media (Lonza) at 37C in 5% CO2 incubator. On the day of experiment, PBMCs and SUDHL-1 target cells were counted and resuspended in X-VIVO 15 (Lonza, phenol red) serum free medium. Antibody dilutions were prepared starting with 10 ug/ml with 5 fold serial dilutions. Target cells were then plated in a 96 well 50 μL/well in a 96-well white flat bottom opaque plate (CORNING Ref #3917) and 10 μL/well antibody dilution was added to the target cells and incubated at 37° C. and 5% CO2 incubater for 30 min. After the incubation, PBMCs were added to the target cells (50 μL/well). For control target cells for spontaneous and max LDH release calculation, 50 μL/well of assay medium (X-VIVO15, phenol red free) was added. The co-incubation (ADCC induction) was done by incubating the plate for 4 h at 37° C. and 5% CO2 incubater 45 min before the co-incubation completed, 10% Triton X100 solution (in PBS) was added to the wells to calculate Max LDH release (10 μL/well, 11× dilution factor). After the incubation, 50 μL/well of the reaction substrate was plated in a 96-well plate (clear flat bottom) and then 50 μL/well of the supernatant of the assay plate was transferred to the reaction substrate. The plate was developed in the dark at RT for 30 min. After the incubation, 50 μL/well of stop solution was added and absorbance 490 nm and 680 nm was measured by using a plate reader (SpectraMax iD3 Plate Reader, Molecular Devices).

Additionally, function antibody-dependent cell phagocytosis (ADCP) assays will also be performed to test activity of macrophages to phagocytose Treg cells upon antibody binding. This may be an additional mechanism of action for Treg depletion. Fc receptor FCyRIIa (CD32b) on macrophages is thought to be the dominant inducer for ADCP. In this assay, primary Tregs will be used as target cells with human monocyte-derived macrophages as effector cells. PBMCs will be isolated from leukocyte reduction system chambers (Stanford Blood Center) for monocyte isolation using CD14 microbeads (Miltenyi Biotec) and for Tregs using Regulatory Human CD4+CD25+ T cell kit (Dynabeads) and Human Treg Cell Differentiation kit (R&D Systems). Monocytes will be cultured for 5-7 days with human serum or M-CSF in media. At days 5-7, macrophages will be cocultured with pre-labeled Tregs (labeled with a fixable viability dye (Invitrogen)) for 2-4 hours at 10 to 1 effector to target ratio with addition of anti-CD25 antibodies and controls, and fixed with fixation buffer (BD Bioscienes). Macrophages will be stained with CD14 to identify macrophages from labeled Tregs, and analyzed for the phagocytic population defined as CD14+ and Treg labeled+ population using flow cytometry. Anti-CD25 antibodies are expected to also induce ADCP.

### Results

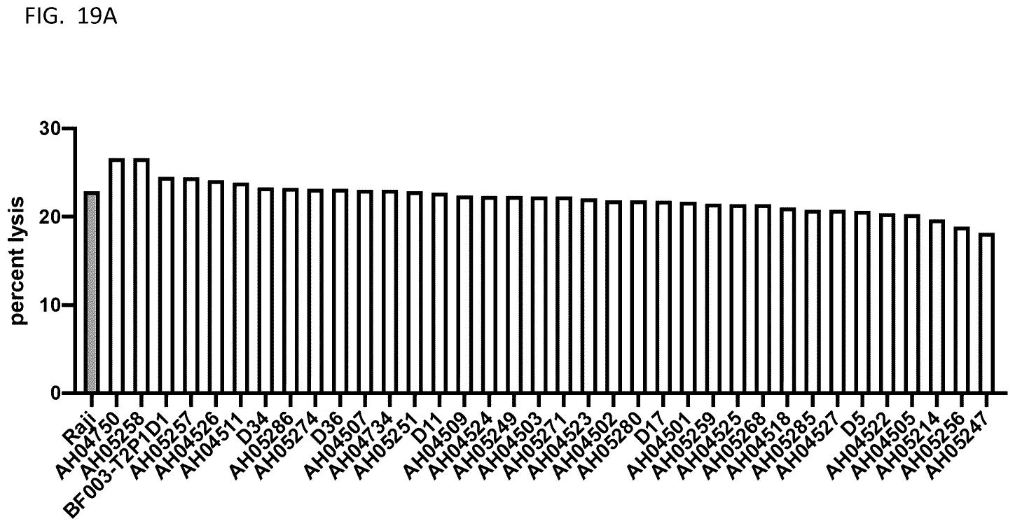

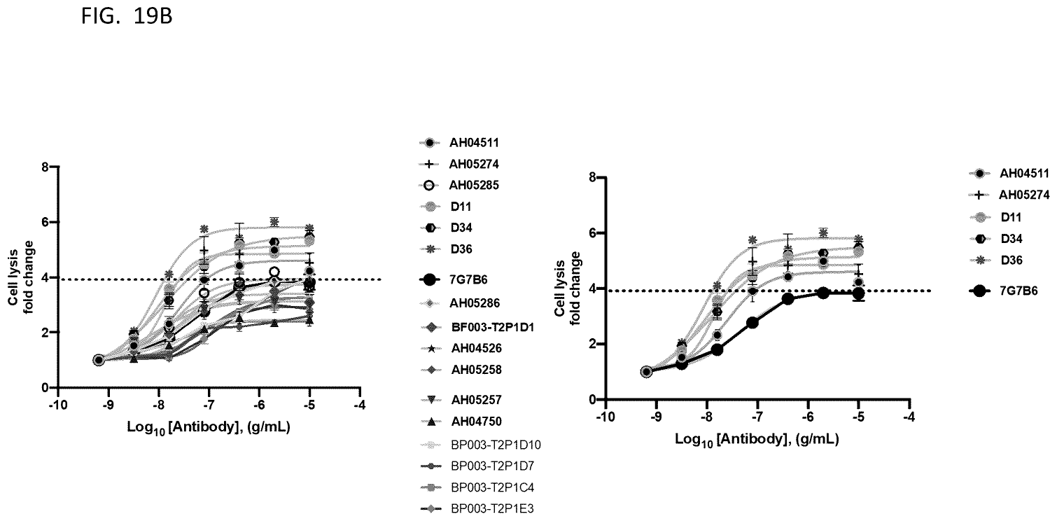

FIGS. 19A-19B show functional ADCC activity of all reformatted clones. In the first screen, clones bound to CD25+ cells, and ADCC was induced with cell killing ranging from 18.2% to 26.7% lysis (Raji cell killing is used as a reference; (FIG. 19A) To verify these findings, the assay was repeated with a dose response curve for select clones. The results show a range of ADCC activity from 2 to 6-fold over baseline lysis, with clones AH04511, AH05274, D11, D34, D36 eliciting the most potent ADCC and with greater potency than the 7G7B6 antibody, as measured by fold change over % background lysis.

Subsequent studies will include additional donors of PBMCs and NK cells as effectors for functional ADCC killing assays.

### Example 8: Functional Characterization of CD25 Antibodies

### Characterization, In Vitro

Subsequent in vitro characterizations will include T-cell activation and Treg suppression studies to evaluate the impact of CD25 antibodies on T-effector cell responses in the absence and presence of Treg cells. Readouts for activation will include intracellular granzyme B, proliferation and cytokine release (e.g. IL-2, IFNγ, TNF-α). Specifically, human primary conventional T cells (T conv cells) from healthy donors and donors with specific antigen responses (e.g. human cytomegalovirus or influenza flu antigens) will be labeled with a cell proliferation dye (e.g. ThermoFisher) and treated with varying concentrations of CD25 and control antibodies (1-10 ug/mL), followed by activation using CD3/CD28 beads and incubated with and without Tregs for incubation at 37° C., 5% CO2 for 48-72 hours. To evaluate T cell activation, supernatants will be collected for cytokine analysis and cells will be stained with fixable viability dye (e.g. Thermofisher) and with surface anti human T cell markers: CD3, CD4, CD8, CD45RA, CD25, followed by fixation and permeabilization for staining of intracellular Granzyme B and Foxp3. Cells will be analyzed using flow cytometry for Granzyme B positive and proliferating cells.

### Characterization, In Vivo

In vivo activity of CD25 antibodies using a mouse xenograft tumor model. These experiments will help to distinguish differences in CD25 antibodies based on CD25+ tumor depletion via ADCC and/or ADCP. Candidate antibodies will be produced as mouse IgG2a isotype antibodies and used in immunodeficient RAG−/− knockout mice (with functional NK and APC cells, but no mature B or T lymphocytes). Animals will be subcutaneously engrafted with CD25+ human cell lines (e.g SUDHL-1, a CD25+ anaplastic large cell lymphoma). Various size tumors (palpable, 100-500 mm3) may be used to discern the differences between antibodies. Animals will be treated with different doses of CD25 antibodies (e.g. 1-10 mg/kg, 3× weekly or once daily) and monitored for changes in body weight and TGI.

### Drug Efficacy and MOA Studies in Mouse Xenograft Tumor Models in Humanized Mice.

The more effective antibodies for tumor growth inhibition via Treg depletion and increased intratumoral Teff/Treg ratio with increased T effector cell activity will be tested in various human tumor models (e.g. liver, breast, melanoma, gastric, NSCLC and colon cancer) to establish efficacy and mechanism of action. Some studies may include using the triple negative breast cancer cell line MDA-MB-231, a gastric cell line, melanoma cell line A375 and liver cancer cell line Huh-7 as well as human PDX models that have been well characterized and have either shown correlations between infiltrated Tregs and tumor growth and/or have been treated with PD-1 antibody combinations in humanized mice engrafted with human PBMCs and/or CD34+ cells. In these models, tumors will be subcutaneously engrafted until tumors reach a range of sizes from palpable to 100-500 mm3, and treated with various doses of CD25 antibodies and dosing schedules (e.g. 1-10 mg/kg, 3× weekly or once daily). Animal health scoring, bodyweight, tumor growth as well as immunophenotyping of blood and tumors will be performed to characterize tumor and immune cell composition and tumor infilitrating cells, and cytokine secretion using flow cytometry, Meso Scale Discovery multiplex plates and histology.

Combination studies in mouse xenograft tumor models in humanized mice. Once the lead candidate and indication is established based on efficacy and MOA studies, we will perform combination studies using a wide range of agents to increase immunogenicity and activation of other pathways for immune activation (e.g. chemotherapy, checkpoint inhibitors, TLR agonists, vaccines). Combination experiments will be conducted in similar tumor models used in efficacy and MOA studies.

All patents, patent applications, publications, documents, web links, and articles cited herein are incorporated herein by reference in their entireties.

What is claimed is:

1. A method of treating a subject in need thereof comprising:
administering to the subject a therapeutically effective amount of a monoclonal antibody or binding fragment thereof that binds to human CD25 comprising:

| ID | CDRH1 | CDRH2 | CDRH3 |
|---|---|---|---|
| D11 | GYTFTDYA | ISTYSGDA | ARGVTFDY |
| | (SEQ ID NO: | (SEQ ID NO: | (SEQ ID NO: |
| | 19) | 21) | 23) |
| D17 | GYTFTNYW | IYPGGGYT | ARVTPAS |
| | (SEQ ID NO: | (SEQ ID NO: | (SEQ ID NO: |
| | 4) | 6) | 8) |
| D34 | GYTFTDYA | ISTYSGDV | ARGVTFDS |
| | (SEQ ID NO: | (SEQ ID NO: | (SEQ ID NO: |
| | 19) | 46) | 48) |
| D36 | GYTFTDYA | ISTYSGDA | ARGVTFDY |
| | (SEQ ID NO: | (SEQ ID NO: | (SEQ ID NO: |
| | 19) | 21) | 23) |
| D5 | GYTFTNYW | IYPGGGYT | ARVTPAS |
| | (SEQ ID NO: | (SEQ ID NO: | (SEQ ID NO: |
| | 4) | 6) | 8) |
| AHH03702 | GFTFSSYA | ISSGGST | ARGEIWGKA |
| | (SEQ ID NO: | (SEQ ID NO: | WFAY (SEQ |
| | 151) | 224) | ID NO: 297) |
| AHH03703 | GYTLTDYS | INTETGEP | AWGNHY |
| | (SEQ ID NO: | (SEQ ID NO: | (SEQ ID NO: |
| | 152) | 225) | 298) |
| AHH03704 | GYTFTSYV | INPYNDGT | ARDGYYVG |
| | (SEQ ID NO: | (SEQ ID NO: | PAY (SEQ ID |
| | 153) | 226) | NO: 299) |
| AHH03705 | GFAFSSYD | ISSGGGST | ARNYRSWF |
| | (SEQ ID NO: | (SEQ ID NO: | AY (SEQ ID |
| | 154) | 227) | NO: 300) |
| AHH03706 | GYAFTNYL | INPGSGGT | ARKGSLTGV |
| | (SEQ ID NO: | (SEQ ID NO: | LAY (SEQ ID |
| | 155) | 228) | NO: 301) |
| AHH03707 | GYIFTNYW | IDPSDSET | ARRGLRAW |
| | (SEQ ID NO: | (SEQ ID NO: | FAY (SEQ ID |
| | 156) | 229) | NO: 302) |
| AHH03708 | GYTFTSYW | INPSTGYT | ARLDYYGSS |
| | (SEQ ID NO: | (SEQ ID NO: | RGFAY (SEQ |
| | 157) | 230) | ID NO: 303) |
| AHH03709 | GYTFTSYY | INPSNGGT | TNGGGWY |
| | (SEQ ID NO: | (SEQ ID NO: | (SEQ ID NO: |
| | 158) | 231) | 304) |
| AHH03711 | GYTFTNYG | INTYTGEP | ASYYDSTYV |
| | (SEQ ID NO: | (SEQ ID NO: | GFAY (SEQ |
| | 159) | 232) | ID NO: 305) |
| AHH03712 | GFTFSDYY | ISNGGGST | ASPLGYDGF |
| | (SEQ ID NO: | (SEQ ID NO: | AY (SEQ ID |
| | 160) | 233) | NO: 306) |
| AHH03713 | GFSLTSYG | IWAGGST | ARGAYFDY |
| | (SEQ ID NO: | (SEQ ID NO: | (SEQ ID NO: |
| | 161) | 234) | 307) |
| AHH03714 | GYTFTSYW | IDPYDSET | ARSPAYYGN |
| | (SEQ ID NO: | (SEQ ID NO: | LWFAY (SEQ |
| | 157) | 235) | ID NO: 308) |
| AHH03715 | GYTFTSYT | INPSSGYT | ARWDGAY |
| | (SEQ ID NO: | (SEQ ID NO: | (SEQ ID NO: |
| | 162) | 236) | 309) |
| AHH03716 | GFTFSSYA | ISSGGSYT | ARGGMITPF |
| | (SEQ ID NO: | (SEQ ID NO: | AY (SEQ ID |
| | 151) | 237) | NO: 310) |
| AHH03717 | GFSLSTSGM | IWWNDDK | ARIGGNDGY |
| | S (SEQ ID | (SEQ ID NO: | YWYFDV |
| | NO: 163) | 238) | (SEQ ID NO: |
| | | | 311) |
| AHH03718 | GFTFSDAW | IRSKANNH | TPQFAY |
| | (SEQ ID NO: | AT (SEQ ID | (SEQ ID NO: |
| | 164) | NO: 239) | 312) |
| AHH03719 | GFTFSSYG | INSNGGST | ASHYDEGY |
| | (SEQ ID NO: | (SEQ ID NO: | (SEQ ID NO: |
| | 165) | 240) | 313) |
| AHH03720 | GFTFSNYW | IRLKSNNYA | TGSDY (SEQ |
| | (SEQ ID NO: | T (SEQ ID | ID NO: 314) |
| | 166) | NO: 241) | |
| AHH03721 | GDTFSSYV | FNPYSDDI | GSGYDGYY |
| | (SEQ ID NO: | (SEQ ID NO: | DWFAC (SEQ |
| | 167) | 242) | ID NO: 315) |
| AHH03722 | GYSFTKNG | INTYTGEP | AREPKTLDY |
| | (SEQ ID NO: | (SEQ ID NO: | (SEQ ID NO: |
| | 168) | 232) | 316) |
| AHH03724 | GYTFTSYW | IDPSDSET | ANWAWFAY |
| | (SEQ ID NO: | (SEQ ID NO: | (SEQ ID NO: |
| | 157) | 229) | 317) |
| AHH03726 | GYTFNSHW | IDPYDSET | ARPYDYDGF |
| | (SEQ ID NO: | (SEQ ID NO: | AY (SEQ ID |
| | 169) | 235) | NO: 318) |
| AHH03727 | GYTFTNHH | INPYNDYT | ADGDYYFD |
| | (SEQ ID NO: | (SEQ ID NO: | Y (SEQ ID |
| | 170) | 243) | NO: 319) |
| AHH03728 | GLTFSSYG | ISSGGSYI | ARQDDGYY |
| | (SEQ ID NO: | (SEQ ID NO: | RIFDY (SEQ |
| | 171) | 244) | ID NO: 320) |
| AHH03729 | GFTFNDAW | IRSKANNH | TNYGSNPLD |
| | (SEQ ID NO: | AT (SEQ ID | Y (SEQ ID |
| | 172) | NO: 239) | NO: 321) |
| AHH03730 | GFSLTSYG | IWAGGST | AREGTGPWF |
| | (SEQ ID NO: | (SEQ ID NO: | AY (SEQ ID |
| | 161) | 234) | NO: 322) |
| AHH03731 | GYSITSGYS | IHYSGST | ARDPPFAY |
| | (SEQ ID NO: | (SEQ ID NO: | (SEQ ID NO: |
| | 173) | 245) | 323) |
| AHH03733 | GYTFTSYW | IDPSDSYT | AREEITAWF |
| | (SEQ ID NO: | (SEQ ID NO: | AY (SEQ ID |
| | 157) | 246) | NO: 324) |
| AHH03734 | GYTFTDYE | IHPGSGGT | TRNGNGNW |
| | (SEQ ID NO: | (SEQ ID NO: | YFDV (SEQ |
| | 174) | 247) | ID NO: 325) |
| AHH03737 | GYAFSSYW | IYPGDGDT | ARSGYRYDA |
| | (SEQ ID NO: | (SEQ ID NO: | VFAY (SEQ |
| | 175) | 248) | ID NO: 326) |
| AHH03738 | GFTFSSYG | INSNGGST | ARGGNPY |
| | (SEQ ID NO: | (SEQ ID NO: | (SEQ ID NO: |
| | 165) | 240) | 327) |
| AHH03739 | GFTFSGFW | INSDGSAI | MRYGSSYW |
| | (SEQ ID NO: | (SEQ ID NO: | YFD (SEQ ID |
| | 176) | 249) | NO: 328) |
| AHH03740 | GFTFSSYW | IRLKSDNYA | TRYYYGES |
| | (SEQ ID NO: | T (SEQ ID | (SEQ ID NO: |
| | 177) | NO: 250) | 329) |
| AHH03742 | GFTFSSYG | ISSGGSYT | ARHYYDYD |
| | (SEQ ID NO: | (SEQ ID NO: | YWYFDV |
| | 165) | 237) | (SEQ ID NO: |
| | | | 330) |
| AHH03743 | GYTFTSYW | IYPGSGST | TRSGVEGLL |
| | (SEQ ID NO: | (SEQ ID NO: | HWYFDV |
| | 157) | 251) | (SEQ ID NO: |
| | | | 331) |
| AHH03746 | GFTFSSYW | IRLKSDNYA | TCDYDGGA |
| | (SEQ ID NO: | T (SEQ ID | WFAY (SEQ |
| | 177) | NO: 250) | ID NO: 332) |
| AHH03748 | GYTFTSYW | IDPSNSET | ARCDGYYD |
| | (SEQ ID NO: | (SEQ ID NO: | GLDY (SEQ |
| | 157) | 252) | ID NO: 333) |
| AHH03749 | GYTFTSYT | INPSSGYT | AREGKNWY |
| | (SEQ ID NO: | (SEQ ID NO: | FDV (SEQ ID |
| | 162) | 236) | NO: 334) |
| AHH03750 | GYTFTSYW | INPSNGGT | ARRIYRTLD |
| | (SEQ ID NO: | (SEQ ID NO: | Y (SEQ ID |
| | 157) | 231) | NO: 335) |
| AHH03751 | GYTFTSYN | IYPGNGDT | TRSGGNLWF |
| | (SEQ ID NO: | (SEQ ID NO: | AY (SEQ ID |
| | 178) | 253) | NO: 336) |
| AHH03752 | GYTFSSYW | ILPGSGST | ARRTYYGN |
| | (SEQ ID NO: | (SEQ ID NO: | AWFAY (SEQ |
| | 179) | 254) | ID NO: 337) |
| AHH03753 | GFTFTDYY | IRNKANGY | ARDKRITTV |
| | (SEQ ID NO: | TT (SEQ ID | EAWFAY |
| | 180) | NO: 255) | (SEQ ID NO: |
| | | | 338) |
| AHH03754 | GFTFSSYA | ISSGGST | ARGYGSSFA |
| | (SEQ ID NO: | (SEQ ID NO: | Y (SEQ ID |
| | 151) | 224) | NO: 339) |
| AHH03755 | GYTFTSYY | INPSNSGT | TRGGDYDAS |
| | (SEQ ID NO: | (SEQ ID NO: | WFAY (SEQ |
| | 158) | 256) | ID NO: 340) |
| AHH03756 | GYTLTDYV | IYPGSGST | ARRTARAFD |
| | (SEQ ID NO: | (SEQ ID NO: | Y (SEQ ID |
| | 181) | 251) | NO: 341) |
| AHH03757 | GFTFSSYA | ISSGGSYT | ARRIGYDGG |
| | (SEQ ID NO: | (SEQ ID NO: | GSWFAY |
| | 151) | 237) | (SEQ ID NO: |
| | | | 342) |
| H03758 | ITYA (SEQ | NNYVT (SEQ | RRCV (SEQ ID |
| | ID NO: 182) | ID NO: 257) | NO: 343) |
| H03759 | SNYR (SEQ | DNYGA (SEQ | FAY (SEQ ID |
| | ID NO: 183) | ID NO: 258) | NO: 344) |
| AHH03760 | GYTFTDYE | IHPGSGGT | TRSDYGSSY |
| | (SEQ ID NO: | (SEQ ID NO: | EFAY (SEQ |
| | 174) | 247) | ID NO: 531) |
| AHH03765 | GYSITSDYA | ISYSGST | ARSRGNYFD |
| | (SEQ ID NO: | (SEQ ID NO: | Y (SEQ ID |
| | 482) | 506) | NO: 532) |
| AHH03767 | GYTFTSYT | INPSSGYT | ARSGLRQA |
| | (SEQ ID NO: | (SEQ ID NO: | WFAY (SEQ |
| | 162) | 236) | ID NO: 533) |
| AHH03768 | GFTFSNYR | ITVKSDNY | SRLFAY |
| | (SEQ ID NO: | GA (SEQ ID | (SEQ ID NO: |
| | 183) | NO: 258) | 534) |
| AHH03770 | GYTFTSYW | INPSNGGT | TITGFDV |
| | (SEQ ID NO: | (SEQ ID NO: | (SEQ ID NO: |
| | 157) | 231) | 535) |
| AHH03771 | GYTFTTYW | IFPGTGTT | ARGGYYNSS |
| | (SEQ ID NO: | (SEQ ID NO: | PFAY (SEQ |
| | 483) | 507) | ID NO: 536) |
| AHH03772 | GFTFTDYY | IRNKANGY | ARDGEVRRA |
| | (SEQ ID NO: | TT (SEQ ID | LAY (SEQ ID |
| | 180) | NO: 255) | NO: 537) |
| AHH03773 | GYTFTSST | INPSSGYT | VRHYYFDY |
| | (SEQ ID NO: | (SEQ ID NO: | (SEQ ID NO: |
| | 484) | 236) | 538) |
| AHH03774 | GFTFSSYT | ISSGGGYT | TRVSAKYFD |
| | (SEQ ID NO: | (SEQ ID NO: | V (SEQ ID |
| | 485) | 508) | NO: 539) |
| AHH03776 | GYTFTNFY | INPSNGGT | TRSYYDYD |
| | (SEQ ID NO: | (SEQ ID NO: | WYFDV (SEQ |
| | 486) | 231) | ID NO: 540) |
| AHH03777 | GYTFTSYW | IYPSDSYT | TRQNYYGSS |
| | (SEQ ID NO: | (SEQ ID NO: | HWYFDV |
| | 157) | 509) | (SEQ ID NO: |
| | | | 541) |
| AHH03778 | GYTFTSYW | IDPSDSET | ANWAWFAY |
| | (SEQ ID NO: | (SEQ ID NO: | (SEQ ID NO: |
| | 157) | 229) | 317) |
| AHH03779 | GYTFTDYS | INTETGEP | ASFYYGNFA |
| | (SEQ ID NO: | (SEQ ID NO: | YYFDY (SEQ |
| | 487) | 225) | ID NO: 542) |
| AHH03780 | GFTFSNYW | IRLKSNNYA | TRIYDSGSSY |
| | (SEQ ID NO: | T (SEQ ID | TWYFDV |
| | 166) | NO: 241) | (SEQ ID NO: |
| | | | 543) |
| AHH03782 | GFSLSTSGM | IWWNDDK | ARIGGNDGY |
| | S (SEQ ID | (SEQ ID NO: | YWYFDV |
| | NO: 163) | 238) | (SEQ ID NO: |
| | | | 311) |
| AHH03783 | GYTFTSYW | INPSNGRT | ARDSSGYGA |
| | (SEQ ID NO: | (SEQ ID NO: | Y (SEQ ID |
| | 157) | 510) | NO: 544) |
| AHH03784 | GYTFTSYW | INPSTGYT | ARYDGYYY |
| | (SEQ ID NO: | (SEQ ID NO: | FDY (SEQ ID |
| | 157) | 230) | NO: 545) |
| AHH03785 | GYTFTSYW | IYPSDSYT | TSHYYGRA |
| | (SEQ ID NO: | (SEQ ID NO: | WFAY (SEQ |
| | 157) | 509) | ID NO: 546) |
| AHH03786 | GYTFTRYY | INPSNGGT | TKGGFYDFF |
| | (SEQ ID NO: | (SEQ ID NO: | AY (SEQ ID |
| | 488) | 231) | NO: 547) |
| AHH03787 | GFTFSSYG | INSNGGST | ASLAY (SEQ |
| | (SEQ ID NO: | (SEQ ID NO: | ID NO: 548) |
| | 165) | 240) | |
| AHH03788 | GYTFTDYY | IYPGSGNT | ARVYSGFDV |
| | (SEQ ID NO: | (SEQ ID NO: | (SEQ ID NO: |
| | 489) | 511) | 549) |
| AHH03790 | GYTFTSYY | IYPGDGST | ARGDGYFA |
| | (SEQ ID NO: | (SEQ ID NO: | WFAY (SEQ |
| | 158) | 512) | ID NO: 550) |
| AHH03791 | GYTFSSYW | ILPGSGST | ARSAHRYDA |
| | (SEQ ID NO: | (SEQ ID NO: | WFAY (SEQ |
| | 179) | 254) | ID NO: 551) |
| AHH03792 | GYTFTNYY | INPTNGGT | TRGMAYRY |
| | (SEQ ID NO: | (SEQ ID NO: | DGAGWFAY |
| | 490) | 513) | (SEQ ID NO: |
| | | | 552) |
| AHH03793 | GYTFTSYW | IDPYDSET | ARGGRGTW |
| | (SEQ ID NO: | (SEQ ID NO: | FAY (SEQ ID |
| | 157) | 235) | NO: 553) |
| AHH03794 | GFTFTDYY | IRNKANGY | ARGWGNWF |
| | (SEQ ID NO: | TT (SEQ ID | AY (SEQ ID |
| | 180) | NO: 255) | NO: 554) |
| AHH03795 | GFTFSSFG | ISGGGGTI | ARWRGGYF |
| | (SEQ ID NO: | (SEQ ID NO: | DY (SEQ ID |
| | 491) | 514) | NO: 555) |
| AHH03797 | GYTFTSYW | IYPSDSYT | TRTGGSTMT |
| | (SEQ ID NO: | (SEQ ID NO: | PWFAY (SEQ |
| | 157) | 509) | ID NO: 556) |
| AHH03799 | GFSLSTSGM | IYWDDDK | ARRAGDYG |
| | G (SEQ ID | (SEQ ID NO: | NPFPY (SEQ |
| | NO: 492) | 515) | ID NO: 557) |
| >AH04501- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04515- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04502- | GYTFTNY | DIHPGGGY | SRNFAY |
| VH | (SEQ ID NO: | TNYNEKFK | (SEQ ID NO: |
| | 990) | G (SEQ ID | 1379) |
| | | NO: 696) | |
| >AH04512- | GYTFTNY | DIHPGGGY | SRNFAY |
| VH | (SEQ ID NO: | TNYNEKFK | (SEQ ID NO: |
| | 990) | G (SEQ ID | 1379) |
| | | NO: 696) | |
| >AH04522- | GYTFTNY | DIHPGGGY | SRNFAY |
| VH | (SEQ ID NO: | TNYNEKFK | (SEQ ID NO: |
| | 990) | G (SEQ ID | 1379) |
| | | NO: 696) | |
| >AH04513- | GYTFTNY | DIHPGGGY | SRNFAY |
| VH | (SEQ ID NO: | TNYNEKFK | (SEQ ID NO: |
| | 990) | G (SEQ ID | 1379) |
| | | NO: 696) | |
| >AH04523- | GYTFTNY | DIHPGGSY | SRNFAK |
| VH | (SEQ ID NO: | TNYNENFK | (SEQ ID NO: |
| | 990) | G (SEQ ID | 1377) |
| | | NO: 697) | |
| >AH04503- | GYTFSNY | DIHPGGGYI | SRNFAN |
| VH | (SEQ ID NO: | NYNEKFTG | (SEQ ID NO: |
| | 985) | (SEQ ID NO: | 1378) |
| | | 695) | |
| >AH04529- | GYTFSNY | DIHPGGGYI | SRNFAN |
| VH | (SEQ ID NO: | NYNEKFTG | (SEQ ID NO: |
| | 985) | (SEQ ID NO: | 1378) |
| | | 695) | |
| >AH04510- | GYTFSNY | DIHPGGGYI | SRNFAN |
| VH | (SEQ ID NO: | NYNEKFTG | (SEQ ID NO: |
| | 985) | (SEQ ID NO: | 1378) |
| | | 695) | |
| >AH04528- | GYTFSNY | DIHPGGGYI | SRNFAN |
| VH | (SEQ ID NO: | NYNEKFTG | (SEQ ID NO: |
| | 985) | (SEQ ID NO: | 1378) |
| | | 695) | |
| >AH04511- | GYTFSNY | DIHPGGGYI | SRNFAN |
| VH | (SEQ ID NO: | NYNEKFTG | (SEQ ID NO: |
| | 985) | (SEQ ID NO: | 1378) |
| | | 695) | |
| >AH04504- | GYTFTKY | DIHPGGGYI | SRNFAN |
| VH | (SEQ ID NO: | NYNEKFTG | (SEQ ID NO: |
| | 989) | (SEQ ID NO: | 1378) |
| | | 695) | |
| >AH04520- | GYTFTKY | DIHPGGGYI | SRNFAN |
| VH | (SEQ ID NO: | NYNEKFTG | (SEQ ID NO: |
| | 989) | (SEQ ID NO: | 1378) |
| | | 695) | |
| >AH04507- | GYTFTNY | DIHPGGGYI | SRNFAK |
| VH | (SEQ ID NO: | DYNEKFTG | (SEQ ID NO: |
| | 990) | (SEQ ID NO: | 1377) |
| | | 694) | |
| >AH04527- | GYTFTNY | DIHPGGGYI | SRNFAK |
| VH | (SEQ ID NO: | DYNEKFTG | (SEQ ID NO: |
| | 990) | (SEQ ID NO: | 1377) |
| | | 694) | |
| >AH04505- | GYTFTNY | DIHPGGGY | SRNFAY |
| VH | (SEQ ID NO: | TNYNEKFK | (SEQ ID NO: |
| | 990) | G (SEQ ID | 1379) |
| | | NO: 696) | |
| >AH04516- | GYTFTNY | DIHPGGGY | SRNFAY |
| VH | (SEQ ID NO: | TNYNEKFK | (SEQ ID NO: |
| | 990) | G (SEQ ID | 1379) |
| | | NO: 696) | |
| >AH04525- | GYTFTNY | DIHPGGDY | GRNFAY |
| VH | (SEQ ID NO: | TNYNEKFK | (SEQ ID NO: |
| | 990) | G (SEQ ID | 982) |
| | | NO: 693) | |
| >AH04509- | GYTFTNY | DFYPGGDY | SRNFAY |
| VH | (SEQ ID NO: | INYNEKFK | (SEQ ID NO: |
| | 990) | G (SEQ ID | 1379) |
| | | NO: 691) | |
| >AH04521- | GYTFTNY | DFYPGGDY | SRNFAY |
| VH | (SEQ ID NO: | INYNEKFK | (SEQ ID NO: |
| | 990) | G (SEQ ID | 1379) |
| | | NO: 691) | |
| >AH04526- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04514- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04524- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04530- | GYTSTNY | DIHPGGDY | SRNFAY |
| VH | (SEQ ID NO: | SNYNEKFK | (SEQ ID NO: |
| | 996) | G (SEQ ID | 1379) |
| | | NO: 692) | |
| >AH04517- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04506- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04508- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04518- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| AH04734 | GYTFTTST | INPRSGYT | ARHYYFDY |
| | (SEQ ID NO: | (SEQ ID NO: | (SEQ ID NO: |
| | 993) | 1024) | 612) |
| AH04750 | GYTSTAYW | ITPSTGYT | ARGGYFDY |
| | (SEQ ID NO: | (SEQ ID NO: | (SEQ ID NO: |
| | 994) | 1028) | 606) |
| AH05214 | GYTFTSYW | INPSNGRT | ARQLAAY |
| | (SEQ ID NO: | (SEQ ID NO: | (SEQ ID NO: |
| | 157) | 510) | 614) |
| AH05247 | GYTFSNYW | ILPGSGFT | ARGGTSVV |
| | (SEQ ID NO: | (SEQ ID NO: | HFDS (SEQ |
| | 986) | 1021) | ID NO: 604) |
| AH05249 | GYTFSNYW | ILPGSGFT | ARGGTSVV |
| | (SEQ ID NO: | (SEQ ID NO: | HFDS (SEQ |
| | 986) | 1021) | ID NO: 604) |
| AH05251 | GFNIKDYY | IDPDNGET | TVFWYGNN |
| | (SEQ ID NO: | (SEQ ID NO: | YAGFAY |
| | 977) | 1003) | (SEQ ID NO: |
| | | | 1420) |
| AH05256 | GYTFTRYW | INPRTDYT | ARHGYFDY |
| | (SEQ ID NO: | (SEQ ID NO: | (SEQ ID NO: |
| | 992) | 1025) | 611) |
| AH05257 | GYTFTDYA | ISTYSGDA | ARGVTFDY |
| | (SEQ ID NO: | (SEQ ID NO: | (SEQ ID NO: |
| | 19) | 21) | 23) |
| AH05258 | GYTFTDYA | ISTYSGDV | ARGVTFDS |
| | (SEQ ID NO: | (SEQ ID NO: | (SEQ ID NO: |
| | 19) | 46) | 48) |
| AH05259 | GYTFTDYA | ISTYSGDV | ARGVTFDS |
| | (SEQ ID NO: | (SEQ ID NO: | (SEQ ID NO: |
| | 19) | 46) | 48) |
| AH05268 | GYTFTDYA | ISTYSGDA | ARGVTEDY |
| | (SEQ ID NO: | (SEQ ID NO: | (SEQ ID NO: |
| | 19) | 21) | 23) |
| AH05271 | GYTFTDYA | ISTYSGDV | ARGVTFDS |
| | (SEQ ID NO: | (SEQ ID NO: | (SEQ ID NO: |
| | 19) | 46) | 48) |
| AH05274 | GYTFTNYW | IYPGGGYT | ARVTPAS |
| | (SEQ ID NO: | (SEQ ID NO: | (SEQ ID NO: |
| | 4) | 6) | 8) |
| AH05280 | GYTFSNYW | ILPGSGFT | ARGGTSVV |
| | (SEQ ID NO: | (SEQ ID NO: | HFDY (SEQ |
| | 986) | 1021) | ID NO: 605) |
| AH05285 | GYTFTRFW | INPSTDYT | ARGTVVDY |
| | (SEQ ID NO: | (SEQ ID NO: | (SEQ ID NO: |
| | 991) | 1027) | 607) |
| AH05286 | GYTFSNYW | ILPGSGYT | ARGGTSFVH |
| | (SEQ ID NO: | (SEQ ID NO: | FDY (SEQ ID |
| | 986) | 1022) | NO: 602) |
| AH4501 | GYTFTNYW | IHPGGDYS | TSRNFAY |
| | (SEQ ID NO: | (SEQ ID NO: | (SEQ ID NO: |
| | 4) | 1012) | 1419) |
| AH4502 | GYTFTNYW | IHPGGGYT | TSRNFAY |
| | (SEQ ID NO: | (SEQ ID NO: | (SEQ ID NO: |
| | 4) | 1015) | 1419) |
| AH4503 | GYTFSNYW | IHPGGGYI | VSRNFAN |
| | (SEQ ID NO: | (SEQ ID NO: | (SEQ ID NO: |
| | 986) | 1014) | 1445) |
| AH4505 | GYTFTNYW | IHPGGGYT | TSRNFAY |
| | (SEQ ID NO: | (SEQ ID NO: | (SEQ ID NO: |
| | 4) | 1015) | 1419) |
| AH4509 | GYTFTNYW | FYPGGDYI | TSRNFAY |
| | (SEQ ID NO: | (SEQ ID NO: | (SEQ ID NO: |
| | 4) | 973) | 1419) |
| AH4511 | GYTFSNYW | IHPGGGYI | VSRNFAN |
| | (SEQ ID NO: | (SEQ ID NO: | (SEQ ID NO: |
| | 986) | 1014) | 1445) |
| AH4518 | GYTFTNYW | IHPGGDYS | TSRNFAY |
| | (SEQ ID NO: | (SEQ ID NO: | (SEQ ID NO: |
| | 4) | 1012) | 1419) |
| AH4523 | GYTFTNYW | IHPGGSYT | TSRNFAK |
| | (SEQ ID NO: | (SEQ ID NO: | (SEQ ID NO: |
| | 4) | 1016) | 1418) |
| AH4524 | GYTFTNYW | IHPGGDYS | TSRNFAY |
| | (SEQ ID NO: | (SEQ ID NO: | (SEQ ID NO: |
| | 4) | 1012) | 1419) |
| AH4525 | GYTFTNYW | IHPGGDYT | TGRNFAY |
| | (SEQ ID NO: | (SEQ ID NO: | (SEQ ID NO: |
| | 4) | 1013) | 1395) |
| BP003- | GYTFTNYW | IYPGGGYT | ARVTPAS |
| T2P1C4 | (SEQ ID NO: | (SEQ ID NO: | (SEQ ID NO: |
| | 4) | 6) | 8) |
| BP003- | GYTFTNYW | IYPGGGYT | ARVTPAS |
| T2P1D10 | (SEQ ID NO: | (SEQ ID NO: | (SEQ ID NO: |
| | 4) | 6) | 8) |
| BP003- | GFNIKDYY | IDPDNGET | TVFWYGNN |
| T2P1D7 | (SEQ ID NO: | (SEQ ID NO: | YAGFAY |
| | 977) | 1003) | (SEQ ID NO: |
| | | | 1420) |
| BP003- | GYTFTNYW | IHPGGGYT | TSRNFAY |
| T2P1E3 | (SEQ ID NO: | (SEQ ID NO: | (SEQ ID NO: |
| | 4) | 1015) | 1419) |
| BP003- | GYTFTDYA | ISTYSGDA | ARGVTFDY |
| T2P1D1 | (SEQ ID NO: | (SEQ ID NO: | (SEQ ID NO: |
| | 19) | 21) | 23) |
| BL_592 | GYTFTNYW | IHPGGGYT | TSRNFAY |
| 989- | (SEQ ID NO: | (SEQ ID NO: | (SEQ ID NO: |
| 2015_G2_ | 4) | 1015) | 1419) |
| P8_G02 | | | |
| BL_592 | GYTFTSYW | IYPGSGST | TRSGVEGLL |
| 989- | (SEQ ID NO: | (SEQ ID NO: | HWYFD |
| 2016_H2_ | 157) | 251) | (SEQ ID NO: |
| P8_H02 | | | 1416) |
| BL_592 | GYTFTSYW | IYPGSGST | TRWITTDHY |
| 989- | (SEQ ID NO: | (SEQ ID NO: | FDY (SEQ ID |
| 2017_A3_ | 157) | 251) | NO: 1417) |
| P8_A03 | | | |
| BL_592 | GYTFTNYW | IYPGGGYT | ARVSPAS |
| 989- | (SEQ ID NO: | (SEQ ID NO: | (SEQ ID NO: |
| 2018_B3_ | 4) | 6) | 615) |
| P8_B03 | | | |
| BL_592 | GYTFTNYW | IYPGGGYT | ARVTPAS |
| 989- | (SEQ ID NO: | (SEQ ID NO: | (SEQ ID NO: |
| 2019_C3_ | 4) | 6) | 8) |
| P8_C03 | | | |
| BL_592 | GYTFTNYW | IHPGGGYT | TSRNFAY |
| 989- | (SEQ ID NO: | (SEQ ID NO: | (SEQ ID NO: |
| 2021_E3_ | 4) | 1015) | 1419) |
| P8_E03 | | | |
| BL_592 | GYTFTNYW | IHPGGGYT | TSRNFAY |
| 989- | (SEQ ID NO: | (SEQ ID NO: | (SEQ ID NO: |
| 2022_F3_ | 4) | 1015) | 1419) |
| P8_F03 | | | |
| BL_592 | GYTFTSYW | IYPGSGST | TRSGVEGLL |
| 989- | (SEQ ID NO: | (SEQ ID NO: | HWYFD |
| 2023_G3_ | 157) | 251) | (SEQ ID NO: |
| P8_G03 | | | 1416) |
| BL_592 | GYTSTGYW | INPSTGYT | ARGGYFDY |
| 989- | (SEQ ID NO: | (SEQ ID NO: | (SEQ ID NO: |
| 2024_H3_ | 995) | 230) | 606) |
| P8_H03 | | | |
| BL_592 | GYTFTNYW | IYPGGGYT | ARVTPAS |
| 989- | (SEQ ID NO: | (SEQ ID NO: | (SEQ ID NO: |
| 2025_A4_ | 4) | 6) | 8) |
| P8_A04 | | | |
| BL_592 | GYTFTDYA | ISTYSGDV | ARGVTFDS |
| 989- | (SEQ ID NO: | (SEQ ID NO: | (SEQ ID NO: |
| 2026_B4_ | 19) | 46) | 48) |
| P8_B04 | | | |
| BL_592 | GYTFTNYW | IYPGGGYT | ARVTPAS |
| 989- | (SEQ ID NO: | (SEQ ID NO: | (SEQ ID NO: |
| 2027_C4_ | 4) | 6) | 8) |
| P8_C04 | | | |
| BL_592 | GYTFTNYW | IHPGGDYT | TGRNFAY |
| 989- | (SEQ ID NO: | (SEQ ID NO: | (SEQ ID NO: |
| 2029_E4_ | 4) | 1013) | 1395) |
| P8_E04 | | | |
| BL_592 | GYTFTNYW | IHPGGGYT | TSRNFAY |
| 989- | (SEQ ID NO: | (SEQ ID NO: | (SEQ ID NO: |
| 2030_F4_ | 4) | 1015) | 1419) |
| P8_F04 | | | |
| BL_592 | GYTFTNYW | IHPGGGYT | TSRNFAY |
| 989- | (SEQ ID NO: | (SEQ ID NO: | (SEQ ID NO: |
| 2031_G4_ | 4) | 1015) | 1419) |
| P8_G04 | | | |
| BL_592 | GYTFSNYW | IHPGGGYI | VSRNFAN |
| 989- | (SEQ ID NO: | (SEQ ID NO: | (SEQ ID NO: |
| 2032_H4_ | 986) | 1014) | 1445) |
| P8_H04 | | | |
| BL_592 | GYTFTDYA | ISTYSGDV | ARGVTFDS |
| 989- | (SEQ ID NO: | (SEQ ID NO: | (SEQ ID NO: |
| 2033_A5_ | 19) | 46) | 48) |
| P8_A05 | | | |
| BL_592 | GYTFTNYW | IYPGGGYT | ARVTPAS |
| 989- | (SEQ ID NO: | (SEQ ID NO: | (SEQ ID NO: |
| 2034_B5_ | 4) | 6) | 8) |
| P8_B05 | | | |
| BL_592 | GYTFTNYW | IYPGGGYT | ARVTPAS |
| 989- | (SEQ ID NO: | (SEQ ID NO: | (SEQ ID NO: |
| 2035_C5_ | 4) | 6) | 8) |
| P8_C05 | | | |
| BL_592 | GYTFTNYW | IHPGGDYS | TSRNFAY |
| 989- | (SEQ ID NO: | (SEQ ID NO: | (SEQ ID NO: |
| 2037_E5_ | 4) | 1012) | 1419) |
| P8_E05 | | | |
| BL_592 | GYTFTNYW | IHPGGGYI | VSRNFAN |
| 989- | (SEQ ID NO: | (SEQ ID NO: | (SEQ ID NO: |
| 2038_F5_ | 4) | 1014) | 1445) |
| P8_F05 | | | |
| BL_592 | GYTFTNYW | IHPGGGYT | TSRNFAY |
| 989- | (SEQ ID NO: | (SEQ ID NO: | (SEQ ID NO: |
| 2039_G5_ | 4) | 1015) | 1419) |
| P8_G05 | | | |
| BL_592 | GYTFTNYW | IHPGGGYT | TSRNFAY |
| 989- | (SEQ ID NO: | (SEQ ID NO: | (SEQ ID NO: |
| 2040_H5_ | 4) | 1015) | 1419) |
| P8_H05 | | | |
| BL_592 | GYTFTDYA | ISTYSGDA | ARGVTFDY |
| 989- | (SEQ ID NO: | (SEQ ID NO: | (SEQ ID NO: |
| 2041_A6_ | 19) | 21) | 23) |
| P8_A06 | | | |
| BL_592 | GYTFTDYA | ISTYSGDV | ARGVTFDS |
| 989- | (SEQ ID NO: | (SEQ ID NO: | (SEQ ID NO: |
| 2042_B6_ | 19) | 46) | 48) |
| P8_B06 | | | |
| BL_592 | GYTFTDYA | ISTYSGDV | ARGVTFDS |
| 989- | (SEQ ID NO: | (SEQ ID NO: | (SEQ ID NO: |
| 2043_C6_ | 19) | 46) | 48) |
| P8_C06 | | | |
| BL_592 | GYTFTNYW | IYPGGGYT | ARVTPAS |
| 989- | (SEQ ID NO: | (SEQ ID NO: | (SEQ ID NO: |
| 2044_D6_ | 4) | 6) | 8) |
| P8_D06 | | | |
| BL_592 | GYTFTNYW | IHPGGDYS | TSRNFAY |
| 989- | (SEQ ID NO: | (SEQ ID NO: | (SEQ ID NO: |
| 2045_E6_ | 4) | 1012) | 1419) |
| P8_E06 | | | |
| BL_592 | GYTFTNYW | IHPGGDYS | TSRNFAY |
| 989- | (SEQ ID NO: | (SEQ ID NO: | (SEQ ID NO: |
| 2046_F6_ | 4) | 1012) | 1419) |
| P8_F06 | | | |
| BL_592 | GYTFTNYW | IHPGGGYT | TSRNFAY |
| 989- | (SEQ ID NO: | (SEQ ID NO: | (SEQ ID NO: |
| 2047_G6_ | 4) | 1015) | 1419) |
| P8_G06 | | | |
| BL_592 | GYTFTNYW | IHPGGDYS | TSRNFAY |
| 989- | (SEQ ID NO: | (SEQ ID NO: | (SEQ ID NO: |
| 2048_H6_ | 4) | 1012) | 1419) |
| P8_H06 | | | |
| BL_592 | GYTFTDYA | ISTYSGDV | ARGVTFDS |
| 989- | (SEQ ID NO: | (SEQ ID NO: | (SEQ ID NO: |
| 2049_A7_ | 19) | 46) | 48) |
| P8_A07 | | | |
| BL_592 | GYTFTNYW | IYPGGGYT | ARVTPAS |
| 989- | (SEQ ID NO: | (SEQ ID NO: | (SEQ ID NO: |
| 2051_C7_ | 4) | 6) | 8) |
| P8_C07 | | | |
| BL_592 | GFNIKDYY | IDPDNGET | TVFWYGNN |
| 989- | (SEQ ID NO: | (SEQ ID NO: | YAGFAY |
| 2052_D7_ | 977) | 1003) | (SEQ ID NO: |
| P8_D07 | | | 1420) |
| BL_592 | GYTFTNYW | IHPGGDYS | TSRNFAY |
| 989- | (SEQ ID NO: | (SEQ ID NO: | (SEQ ID NO: |
| 2054_F7_ | 4) | 1012) | 1419) |
| P8_F07 | | | |
| BL_592 | GYTFTNYW | INPGGGYT | TSRNFAY |
| 989- | (SEQ ID NO: | (SEQ ID NO: | (SEQ ID NO: |
| 2055_G7_ | 4) | 1023) | 1419) |
| P8_G07 | | | |
| BL_592 | GYTFTNYW | IYPGGGYT | VRVTPAS |
| 989- | (SEQ ID NO: | (SEQ ID NO: | (SEQ ID NO: |
| 2059_C8_ | 4) | 6) | 1443) |
| P8_C08 | | | |
| BL_592 | GYTFTDYA | ISTYSGDA | ARGVTFDY |
| 989- | (SEQ ID NO: | (SEQ ID NO: | (SEQ ID NO: |
| 2060_D8_ | 19) | 21) | 23) |
| P8_D08 | | | |
| BL_592 | GYTFTNYW | IHPGGGYI | VSRNFAN |
| 989- | (SEQ ID NO: | (SEQ ID NO: | (SEQ ID NO: |
| 2061_E8_ | 4) | 1014) | 1445) |
| P8_E08 | | | |
| BL_592 | GYTFTDYA | IITYSGDA | AXGVTFDY |
| 989- | (SEQ ID NO: | (SEQ ID NO: | (SEQ ID NO: |
| 2062_F8_ | 19) | 1020) | 619) |
| P8_F08 | | | |
| BL_592 | GYTFTSYW | IDPYDSET | AREASYYY |
| 989- | (SEQ ID NO: | (SEQ ID NO: | GNAWFA |
| 2063_G8_ | 157) | 235) | (SEQ ID NO: |
| P8_G08 | | | 601) |
| BL_592 | GYTSTNYW | IHPGGDYS | TSRNFAY |
| 989- | (SEQ ID NO: | (SEQ ID NO: | (SEQ ID NO: |
| 2064_H8_ | 997) | 1012) | 1419) |
| P8_H08 | | | |
| BL_592 | GYTFTDYA | ISTYSGDA | ARGVTFDY |
| 989- | (SEQ ID NO: | (SEQ ID NO: | (SEQ ID NO: |
| 2065_A9_ | 19) | 21) | 23) |
| P8_A09 | | | |
| BL_592 | GYAFTNY | IYPGGGYT | ARVTPAS |
| 989- | W (SEQ ID | (SEQ ID NO: | (SEQ ID NO: |
| 2067_C9_ | NO: 984) | 6) | 8) |
| P8_C09 | | | |
| BL_592 | GYTFTDYA | ISTYSGDA | ARGVTFDY |
| 989- | (SEQ ID NO: | (SEQ ID NO: | (SEQ ID NO: |
| 2068_D9_ | 19) | 21) | 23) |
| P8_D09 | | | |
| BL_592 | GYTFTNYW | IHPGGDYS | TSRNFAY |
| 989- | (SEQ ID NO: | (SEQ ID NO: | (SEQ ID NO: |
| 2069_E9_ | 4) | 1012) | 1419) |
| P8_E09 | | | |
| BL_592 | GYTFTDYA | ISTYSGDV | ARGVTFDS |
| 989- | (SEQ ID NO: | (SEQ ID NO: | (SEQ ID NO: |
| 2070_F9_ | 19) | 46) | 48) |
| P8_F09 | | | |
| BL_592 | GYTFTNYW | IHPGGGYT | TSRNFAY |
| 989- | (SEQ ID NO: | (SEQ ID NO: | (SEQ ID NO: |
| 2071_G9_ | 4) | 1015) | 1419) |
| P8_G09 | | | |
| BL_592 | GYTFTNYW | IYPGGGYT | ARVTPAS |
| 989- | (SEQ ID NO: | (SEQ ID NO: | (SEQ ID NO: |
| 2074_ | 4) | 6) | 8) |
| B10_ | | | |
| P8_B10 | | | |
| BL_592 | GYTFTNYW | IYPGGGYT | ARVTPAS |
| 989- | (SEQ ID NO: | (SEQ ID NO: | (SEQ ID NO: |
| 2076_ | 4) | 6) | 8) |
| D10_P8_ | | | |
| D10 | | | |
| BL_592 | GYTFTNYW | IHPGGGYT | TSRNFAY |
| 989- | (SEQ ID NO: | (SEQ ID NO: | (SEQ ID NO: |
| 2077_ | 4) | 1015) | 1419) |
| E10_ | | | |
| P8_E10 | | | |
| BL_592 | GYTFTNYW | IHPGGDYS | TSRNFAY |
| 989- | (SEQ ID NO: | (SEQ ID NO: | (SEQ ID NO: |
| 2078_ | 4) | 1012) | 1419) |
| F10_ | | | |
| P8_F10 | | | |
| BL_592 | GYTFTNYW | FYPGGDYI | TSRNFAY |
| 989- | (SEQ ID NO: | (SEQ ID NO: | (SEQ ID NO: |
| 2079_ | 4) | 973) | 1419) |
| G10_ | | | |
| P8_G10 | | | |
| BL_592 | GYTFTRYW | INPSTGYT | ARWGNFDY |
| 989- | (SEQ ID NO: | (SEQ ID NO: | (SEQ ID NO: |
| 2080_ | 992) | 230) | 617) |
| H10_ | | | |
| P8_H10 | | | |
| BP003- | GYTFTDYA | ISTYSGDV | ARGVTFDS |
| T2P1A7 | (SEQ ID NO: | (SEQ ID NO: | (SEQ ID NO: |
| | 19) | 46) | 48) |
| BP003- | GYTFTNYW | IYPGGGYT | VRVTPAS |
| T2P1C8 | (SEQ ID NO: | (SEQ ID NO: | (SEQ ID NO: |
| | 4) | 6) | 1443) |
| BP003- | GYTFTNYW | IHPGGGYT | TSRNFAY |
| T2P1F4 | (SEQ ID NO: | (SEQ ID NO: | (SEQ ID NO: |
| | 4) | 1015) | 1419) |
| BP003- | GYTFTNYW | IHPGGGYT | TSRNFAY |
| T2P1G2 | (SEQ ID NO: | (SEQ ID NO: | (SEQ ID NO: |
| | 4) | 1015) | 1419) |
| BP003- | GYTFTNYW | IHPGGGYT | TSRNFAY |
| T2P1G4 | (SEQ ID NO: | (SEQ ID NO: | (SEQ ID NO: |
| | 4) | 1015) | 1419) |
| BP003- | GYTFTNYW | INPGGGYT | TSRNFAY |
| T2P1G7 | (SEQ ID NO: | (SEQ ID NO: | (SEQ ID NO: |
| | 4) | 1023) | 1419) |
| BP003- | GYTFTNYW | IHPGGGYT | TSRNFAY |
| T2P1H5 | (SEQ ID NO: | (SEQ ID NO: | (SEQ ID NO: |
| | 4) | 1015) | 1419) |
| BP003T | GYTFTDYA | ISTYSGDA | ARGVTFDY |
| 3P2-1 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-10 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-11 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-12 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-13 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-14 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-15 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-16 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-17 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-18 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-19 | (SEQ ID NO: | (SEQ ID NO: | (SEQ ID NO: |
| | 19) | 21) | 23) |
| BP003T | GYTFTDYA | ISTYSGDA | ARGVTEDY |
| 3P2-2 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-20 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-21 | (SEQ ID NO: | (SEQ ID | (SEQ ID NO: |
| | 19) | NO: 21) | 23) |
| BP003T | GYTFTDYA | ISTYSGDA | ARGVTFDY |
| 3P2-22 | (SEQ ID NO: | (SEQ ID | (SEQ ID NO: |
| | 19) | NO: 21) | 23) |
| BP003T | GYTFTDYA | ISTYSGDV | ARGVTFDS |
| 3P2-23 | (SEQ ID NO: | (SEQ ID | (SEQ ID NO: |
| | 19) | NO: 46) | 48) |
| BP003T | GYTFTDYA | ISTYSGDV | ARGVTFDS |
| 3P2-24 | (SEQ ID NO: | (SEQ ID | (SEQ ID NO: |
| | 19) | NO: 46) | 48) |
| BP003T | GYTFTDYA | ISTYSGDV | ARGVTFDS |
| 3P2-25 | (SEQ ID NO: | (SEQ ID | (SEQ ID NO: |
| | 19) | NO: 46) | 48) |
| BP003T | GYTFTDYA | IXTYSGDV | ARGVTFDS |
| 3P2-26 | (SEQ ID NO: | (SEQ ID | (SEQ ID NO: |
| | 19) | NO: 1029) | 48) |
| BP003T | GYTFTDYG | ISTYSGDV | ARGVTFDS |
| 3P2-27 | (SEQ ID NO: | (SEQ ID | (SEQ ID NO: |
| | 987) | NO: 46) | 48) |
| BP003T | GYTFTRYW | INPRTDYT | ARHGYFDY |
| 3P2-28 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-29 | (SEQ ID NO: | (SEQ ID | (SEQ ID NO: |
| | 992) | NO: 1025) | 611) |
| BP003T | GYTFTDYA | ISTYSGDA | ARGVTFDY |
| 3P2-3 | (SEQ ID NO: | (SEQ ID | (SEQ ID NO: |
| | 19) | NO: 21) | 23) |
| BP003T | GYTFTRYW | INPRTDYT | ARHGYFDY |
| 3P2-30 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-31 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-32 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-33 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-34 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-35 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-36 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-37 | (SEQ ID NO: | (SEQ ID NO: | (SEQ ID NO: |
| | 992) | 1025) | 611) |
| BP003T | GYTFTRYW | INPSSDYT | ARGTVVVD |
| 3P2-38 | (SEQ ID NO: | (SEQ ID | Y (SEQ ID |
| | 992) | NO: 1026) | NO: 608) |
| BP003T | GYTFTRYW | INPSTDYT | VRSPILDY |
| 3P2-39 | (SEQ ID NO: | (SEQ ID | (SEQ ID NO: |
| | 992) | NO: 1027) | 1442) |
| BP003T | GYTFTDYA | ISTYSGDA | ARGVTFDY |
| 3P2-4 | (SEQ ID NO: | (SEQ ID | (SEQ ID NO: |
| | 19) | NO: 21) | 23) |
| BP003T | GYTFTNYW | IYPGGGYT | ARVTPAS |
| 3P2-40 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-41 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-42 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-43 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-44 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-45 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-46 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-47 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-48 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-49 | (SEQ ID NO: | (SEQ ID | (SEQ ID NO: |
| | 4) | NO: 6) | 8) |
| BP003T | GYTFTDYA | ISTYSGDA | ARGVTFDY |
| 3P2-5 | (SEQ ID NO: | (SEQ ID | (SEQ ID NO: |
| | 19) | NO: 21) | 23) |
| BP003T | GYTFTNYW | IYPGGGYT | ARVTPAS |
| 3P2-50 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-51 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-52 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-53 | (SEQ ID NO: | (SEQ ID | (SEQ ID NO: |
| | 4) | NO: 6) | 8) |
| BP003T | GYTFTNYW | IYPGGGYA | ARVTPAS |
| 3P2-54 | (SEQ ID NO: | (SEQ ID | (SEQ ID NO: |
| | 4) | NO: 1035) | 8) |
| BP003T | GYTFTNYW | IYPGGGYT | ARVTPAS |
| 3P2-55 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-56 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-57 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-58 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-59 | (SEQ ID NO: | (SEQ ID | (SEQ ID NO: |
| | 4) | NO: 6) | 8) |
| BP003T | GYTFTDYA | ISTYSGDA | ARGVTFDY |
| 3P2-6 | (SEQ ID NO: | (SEQ ID | (SEQ ID NO: |
| | 19) | NO: 21) | 23) |
| BP003T | GYTFTNYW | IYPGGGYT | ARVTPAS |
| 3P2-60 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-61 | (SEQ ID NO: | (SEQ ID | (SEQ ID NO: |
| | 4) | NO: 6) | 8) |
| BP003T | GYTFSNYW | ILPGSGFT | ARGGTSVV |
| 3P2-62 | (SEQ ID NO: | (SEQ ID | HFDY (SEQ |
| | 986) | NO: 1021) | ID NO: 605) |
| BP003T | GYTFSNYW | ILPGSGFT | ARGGTSVV |
| 3P2-63 | (SEQ ID NO: | (SEQ ID | HFDS (SEQ |
| | 986) | NO: 1021) | ID NO: 604) |
| BP003T | GYTFSNYW | ILPGSGFT | ARGGTSVV |
| 3P2-64 | (SEQ ID NO: | (SEQ ID | HFDY (SEQ |
| | 986) | NO: 1021) | ID NO: 605) |
| BP003T | GYTFSNYW | ILPGSGFT | ARGGTSVV |
| 3P2-65 | (SEQ ID NO: | (SEQ ID | HFDFDY |
| | 986) | NO: 1021) | (SEQ ID NO: |
| | | | 603) |
| BP003T | GFNIKDYY | IDPENGDT | NVITTATTW |
| 3P2-66 | (SEQ ID NO: | (SEQ ID | FAY (SEQ ID |
| | 977) | NO: 1004) | NO: 1165) |
| BP003T | GFNIKDYY | IDPDNGET | TVFWYGNN |
| 3P2-67 | (SEQ ID NO: | (SEQ ID | YAGFAY |
| | 977) | NO: 1003) | (SEQ ID NO: |
| | | | 1420) |
| BP003T | GFNIKDYY | IDPDNGET | TVFWYGNN |
| 3P2-68 | (SEQ ID NO: | (SEQ ID | YAGFAY |
| | 977) | NO: 1003) | (SEQ ID NO: |
| | | | 1420) |
| BP003T | GFNIKDYY | IDPDNGET | TVFWYGNN |
| 3P2-69 | (SEQ ID NO: | (SEQ ID | YAGFAY |
| | 977) | NO: 1003) | (SEQ ID NO: |
| | | | 1420) |
| BP003T | GYTFTDYA | ISTYSGDA | ARGVTFDY |
| 3P2-7 | (SEQ ID NO: | (SEQ ID | (SEQ ID NO: |
| | 19) | NO: 21) | 23) |
| BP003T | GFNIKDYY | IDPDNGET | TVFWYGNN |
| 3P2-70 | (SEQ ID NO: | (SEQ ID | YAGFAY |
| | 977) | NO: 1003) | (SEQ ID NO: |
| | | | 1420) |
| BP003T | GFNIKDYY | IDPDNGET | TVFWYGNN |
| 3P2-71 | (SEQ ID NO: | (SEQ ID | YAGFAY |
| | 977) | NO: 1003) | (SEQ ID NO: |
| | | | 1420) |
| BP003T | GFNIKDYY | IDPDNGET | TVFWYGNN |
| 3P2-72 | (SEQ ID NO: | (SEQ ID | YAGFAY |
| | 977) | NO: 1003) | (SEQ ID NO: |
| | | | 1420) |
| BP003T | GFNIKDYY | IDPDNGET | TVFWYGNN |
| 3P2-73 | (SEQ ID NO: | (SEQ ID | YAGFAY |
| | 977) | NO: 1003) | (SEQ ID NO: |
| | | | 1420) |
| BP003T | GFNIKDYY | IDPDNGET | TVFWYGNN |
| 3P2-74 | (SEQ ID NO: | (SEQ ID | YAGFAY |
| | 977) | NO: 1003) | (SEQ ID NO: |
| | | | 1420) |
| BP003T | GYTFTDYA | ISTYSGDV | ARGVTFDS |
| 3P2-75 | (SEQ ID NO: | (SEQ ID | (SEQ ID NO: |
| | 19) | NO: 46) | 48) |
| BP003T | GYTFTDYA | ISTYSGDA | ARGVTFDY |
| 3P2-8 | (SEQ ID NO: | (SEQ ID | (SEQ ID NO: |
| | 19) | NO: 21) | 23) |
| BP003T | GYTFTDYA | ISTYSGDA | ARGVTFDY |
| 3P2-9 | (SEQ ID NO: | (SEQ ID | (SEQ ID NO: |
| | 19) | NO: 21) | 23) |
| ID | CDRL1 | CDRL2 | CDRL3 |
| D11 | QDISNY | NAK (SEQ | QHHYDTPY |
| | (SEQ ID NO: | ID NO: 28) | T (SEQ ID |
| | 27) | | NO: 30) |
| D17 | QNVGTN | SAS (SEQ | QQYNSYPLT |
| | (SEQ ID NO: | ID NO: 13) | (SEQ ID NO: |
| | 12) | | 42) |
| D34 | ENSYSY | NAK (SEQ | QHHYGTPY |
| | (SEQ ID NO: | ID NO: 28) | T (SEQ ID |
| | 52) | | NO: 54) |
| D36 | ENSYSY | NAK (SEQ | QHHYGTPY |
| | (SEQ ID NO: | ID NO: 28) | T (SEQ ID |
| | 52) | | NO: 54) |
| D5 | QNVGTN | SAS (SEQ | QQYNSYPY |
| | (SEQ ID NO: | ID NO: 13) | T (SEQ ID |
| | 12) | | NO: 15) |
| AHH03702 | QDVSTA | SAS (SEQ | QQHYSTPW |
| | (SEQ ID NO: | ID NO: 13) | T (SEQ ID |
| | 1209) | | NO: 1264) |
| AHH03703 | QDVSTA | SAS (SEQ | QQHYSTPW |
| | (SEQ ID NO: | ID NO: 13) | T (SEQ ID |
| | 1209) | | NO: 1264) |
| AHH03704 | QSLLYSSN | WAS (SEQ | QQYYSYPW |
| | QKNY (SEQ | ID NO: | T (SEQ ID |
| | ID NO: | 1450) | NO: 1294) |
| | 1304) | | |
| AHH03705 | QSLLYSSN | WAS (SEQ | QQYYSYPW |
| | QKNY (SEQ | ID NO: | T (SEQ ID |
| | ID NO: | 1450) | NO: 1294) |
| | 1304) | | |
| AHH03706 | QGISNY | YTS (SEQ | QQYSKLPW |
| | (SEQ ID NO: | ID NO: | T (SEQ ID |
| | 1211) | 1495) | NO: 1289) |
| AHH03707 | KSISKY | SGS (SEQ | QQHNEYPW |
| | (SEQ ID NO: | ID NO: | T (SEQ ID |
| | 1059) | 1365) | NO: 1257) |
| AHH03708 | QSLLDSDG | LVS (SEQ | WQGTHFPR |
| | KTY (SEQ | ID NO: | T (SEQ ID |
| | ID NO: | 1116) | NO: 1464) |
| | 1301) | | |
| AHH03709 | ENVGTY | GAS (SEQ | GQSYSYPYT |
| | (SEQ ID NO: | ID NO: | (SEQ ID NO: |
| | 933) | 975) | 981) |
| AHH03711 | QNVRTA | MAS (SEQ | LQHWNYPY |
| | (SEQ ID NO: | ID NO: | T (SEQ ID |
| | 1247) | 1122) | NO: 1093) |
| AHH03712 | QSVSND | YAS (SEQ | QQDYSSPW |
| | (SEQ ID NO: | ID NO: | T (SEQ ID |
| | 1310) | 1476) | NO: 1251) |
| AHH03713 | QEISGY | AAS (SEQ | LQYISYPRT |
| | (SEQ ID NO: | ID NO: | (SEQ ID NO: |
| | 1210) | 599) | 1107) |
| AHH03714 | QNVGTN | SAS (SEQ | QQYNSYPRT |
| | (SEQ ID NO: | ID NO: 13) | (SEQ ID NO: |
| | 12) | | 1286) |
| AHH03715 | ENIYSN | AAT (SEQ | QHFWGTPPT |
| | (SEQ ID NO: | ID NO: | (SEQ ID NO: |
| | 929) | 600) | 1212) |
| AHH03716 | QSLLDSDG | LVS (SEQ | WQGTHFRT |
| | KTY (SEQ | ID NO: | (SEQ ID NO: |
| | ID NO: | 1116) | 1466) |
| | 1301) | | |
| AHH03717 | QSLVHNNG | KVS (SEQ | SQSTHVPLT |
| | NTY (SEQ | ID NO: | (SEQ ID NO: |
| | ID NO: | 1062) | 1370) |
| | 1305) | | |
| AHH03718 | QDISNY | FTS (SEQ | QQGNTLPRT |
| | (SEQ ID NO: | ID NO: | (SEQ ID NO: |
| | 27) | 972) | 1254) |
| AHH03719 | QDIGSS | ATS (SEQ | LQYASSPYT |
| | (SEQ ID NO: | ID NO: | (SEQ ID NO: |
| | 1199) | 618) | 1101) |
| AHH03720 | QDISNY | YTS (SEQ | QQVYTLPW |
| | (SEQ ID NO: | ID NO: | T (SEQ ID |
| | 27) | 1495) | NO: 1273) |
| AHH03721 | QEISGY | AAS (SEQ | LQYASYPRT |
| | (SEQ ID NO: | ID NO: | (SEQ ID NO: |
| | 1210) | 599) | 1102) |
| AHH03722 | QEISGY | AAS (SEQ | LQYASYPRT |
| | (SEQ ID NO: | ID NO: | (SEQ ID NO: |
| | 1210) | 599) | 1102) |
| AHH03724 | QSVDYDG | AAS (SEQ | QQSNEDPYT |
| | DSY (SEQ | ID NO: | (SEQ ID NO: |
| | ID NO: | 599) | 1270) |
| | 1308) | | |
| AHH03726 | QSVDYDG | AAS (SEQ | QQSNEDPYT |
| | DSY (SEQ | ID NO: | (SEQ ID NO: |
| | ID NO: | 599) | 1270) |
| | 1308) | | |
| AHH03727 | QSLVHSNG | KVS (SEQ | SQSTHVPWT |
| | NTY (SEQ | ID NO: | (SEQ ID NO: |
| | ID NO: | 1062) | 1374) |
| | 1306) | | |
| AHH03728 | QDVNTA | SAS (SEQ | QQHFNSPYT |
| | (SEQ ID NO: | ID NO: 13) | (SEQ ID NO: |
| | 1206) | | 1256) |
| AHH03729 | QSVDYDG | ATS (SEQ | QQSNEDPLT |
| | DSY (SEQ | ID NO: | (SEQ ID NO: |
| | ID NO: | 618) | 1268) |
| | 1308) | | |
| AHH03730 | QDIGSS | ATS (SEQ | LQYASSPWT |
| | (SEQ ID NO: | ID NO: | (SEQ ID NO: |
| | 1199) | 618) | 1100) |
| AHH03731 | SSVSY (SEQ | DTS (SEQ | QQWSSNPPT |
| | ID NO: | ID NO: | (SEQ ID NO: |
| | 1385) | 839) | 1274) |
| AHH03733 | SSIGY (SEQ | DTS (SEQ | HQRGSYPW |
| | ID NO: | ID NO: | T (SEQ ID |
| | 1381) | 839) | NO: 999) |
| AHH03734 | QDVSTA | SAS (SEQ | QQHYSTPW |
| | (SEQ ID NO: | ID NO: 13) | T (SEQ ID |
| | 1209) | | NO: 1264) |
| AHH03737 | ENVATY | GAS (SEQ | GQSYRYPYT |
| | (SEQ ID NO: | ID NO: | (SEQ ID NO: |
| | 932) | 975) | 979) |
| AHH03738 | QSLVHSNG | KVS (SEQ | SQSTHVPT |
| | NTY (SEQ | ID NO: | (SEQ ID NO: |
| | ID NO: | 1062) | 1373) |
| | 1306) | | |
| AHH03739 | ESVDSYGN | LAS (SEQ | QQNNEDPY |
| | SF (SEQ ID | ID NO: | T (SEQ ID |
| | NO: 935) | 1063) | NO: 1266) |
| AHH03740 | QNVGTN | SAS (SEQ | QQYNSYPY |
| | (SEQ ID NO: | ID NO: 13) | T (SEQ ID |
| | 12) | | NO: 15) |
| AHH03742 | ENVGTY | GAS (SEQ | GQSYSYPYT |
| | (SEQ ID NO: | ID NO: | (SEQ ID NO: |
| | 933) | 975) | 981) |
| AHH03743 | QDVSTA | SAS (SEQ | QQHYSTPP |
| | (SEQ ID NO: | ID NO: 13) | WT (SEQ ID |
| | 1209) | | NO: 1262) |
| AHH03746 | QSLLYSNG | QVS (SEQ | LQGTYYPT |
| | KTY (SEQ | ID NO: | WT (SEQ ID |
| | ID NO: | 1344) | NO: 1092) |
| | 1303) | | |
| AHH03748 | QSLVHSNG | KVS (SEQ | SQSTHVPWT |
| | NTY (SEQ | ID NO: | (SEQ ID NO: |
| | ID NO: | 1062) | 1374) |
| | 1306) | | |
| AHH03749 | QDVSTA | SAS (SEQ | QQHYSTPPT |
| | (SEQ ID NO: | ID NO: 13) | (SEQ ID NO: |
| | 1209) | | 1261) |
| AHH03750 | QSLLDSDG | LVS (SEQ | WQGTHFPW |
| | KTY (SEQ | ID NO: | T (SEQ ID |
| | ID NO: | 1116) | NO: 1465) |
| | 1301) | | |
| AHH03751 | QSLLDSDG | LVS (SEQ | WQGAHFPW |
| | KTY (SEQ | ID NO: | T (SEQ ID |
| | ID NO: | 1116) | NO: 1461) |
| | 1301) | | |
| AHH03752 | QSLLDSDG | LVS (SEQ | WQGTHFPQ |
| | KTY (SEQ | ID NO: | T (SEQ ID |
| | ID NO: | 1116) | NO: 1463) |
| | 1301) | | |
| AHH03753 | QNVGSN | SAS (SEQ | QQYDSYPY |
| | (SEQ ID NO: | ID NO: 13) | T (SEQ ID |
| | 1244) | | NO: 1280) |
| AHH03754 | QDIGSS | ATS (SEQ | LQYASSPHT |
| | (SEQ ID NO: | ID NO: | (SEQ ID NO: |
| | 1199) | 618) | 1098) |
| AHH03755 | ENIYSN | AAT (SEQ | QHFWGTPW |
| | (SEQ ID NO: | ID NO: | T (SEQ ID |
| | 929) | 600) | NO: 1213) |
| AHH03756 | QDVSTA | SAS (SEQ | QQHYSTPYT |
| | (SEQ ID NO: | ID NO: 13) | (SEQ ID NO: |
| | 1209) | | 1265) |
| AHH03757 | ENIYSN | AAT (SEQ | QHFWGTPW |
| | (SEQ ID NO: | ID NO: | T (SEQ ID |
| | 929) | 600) | NO: 1213) |
| H03758 | DDD (SEQ ID | (SEQ ID | DNMPLT (SEQ |
| | NO: 1391) | NO: 925) | ID NO: 1094) |
| H03759 | VDYDGDSY | (SEQ ID | NEDPYT (SEQ |
| | (SEQ ID NO: | NO: 599) | ID NO: 1270) |
| | 1308) | | |
| AHH03760 | QSVSND | YAS (SEQ | QQDYSSPPT |
| | (SEQ ID NO: | ID NO: | (SEQ ID NO: |
| | 1310) | 1476) | 1250) |
| AHH03765 | SSVSY (SEQ | DTS (SEQ | QQWSSNPP |
| | ID NO: | ID NO: | YT (SEQ ID |
| | 1385) | 839) | NO: 1275) |
| AHH03767 | QSLLDSDG | LVS (SEQ | WQGTHFPR |
| | KTY (SEQ | ID NO: | T (SEQ ID |
| | ID NO: | 1116) | NO: 1464) |
| | 1301) | | |
| AHH03768 | EDIYNR | GAT (SEQ | QQYWSTPPT |
| | (SEQ ID NO: | ID NO: | (SEQ ID NO: |
| | 924) | 976) | 1292) |
| AHH03770 | QSLLYSSN | WAS (SEQ | QQYYSYP |
| | QKNY (SEQ | ID NO: | (SEQ ID NO: |
| | ID NO: | 1450) | 1293) |
| | 1304) | | |
| AHH03771 | QSLVHSNG | KVS (SEQ | SQSTHVYT |
| | NTY (SEQ | ID NO: | (SEQ ID NO: |
| | ID NO: | 1062) | 1376) |
| | 1306) | | |
| AHH03772 | ENIYSY | NAK (SEQ | QHHYGTPW |
| | (SEQ ID NO: | ID NO: 28) | T (SEQ ID |
| | 930) | | NO: 1217) |
| AHH03773 | QSLVHSNG | KVS (SEQ | SQSTHVPT |
| | NTY (SEQ | ID NO: | (SEQ ID NO: |
| | ID NO: | 1062) | 1373) |
| | 1306) | | |
| AHH03774 | SSVSY (SEQ | DTS (SEQ | QQWSSYPY |
| | ID NO: | ID NO: | T (SEQ ID |
| | 1385) | 839) | NO: 1278) |
| AHH03776 | TDIDDD | EGN (SEQ | LQSDNMPY |
| | (SEQ ID NO: | ID NO: | T (SEQ ID |
| | 1391) | 925) | NO: 1095) |
| AHH03777 | QSLLDSDG | LVS (SEQ | WQGTFSSH |
| | KTY (SEQ | ID NO: | (SEQ ID NO: |
| | ID NO: | 1116) | 1462) |
| | 1301) | | |
| AHH03778 | QDVITA | SAS (SEQ | QQHYSTPRT |
| | (SEQ ID NO: | ID NO: 13) | (SEQ ID NO: |
| | 1205) | | 1263) |
| AHH03779 | QEISGY | AAS (SEQ | LQYASYPRT |
| | (SEQ ID NO: | ID NO: | (SEQ ID NO: |
| | 1210) | 599) | 1102) |
| AHH03780 | ESVDSYGN | RAS (SEQ | QQSNEDPRT |
| | SF (SEQ ID | ID NO: | (SEQ ID NO: |
| | NO: 935) | 1346) | 1269) |
| AHH03782 | QSLVHSNG | KVS (SEQ | SQSTHVPWT |
| | NTY (SEQ | ID NO: | (SEQ ID NO: |
| | ID NO: | 1062) | 1374) |
| | 1306) | | |
| AHH03783 | QSVDYDG | AAS (SEQ | QQSNEDPYT |
| | DSY (SEQ | ID NO: | (SEQ ID NO: |
| | ID NO: | 599) | 1270) |
| | 1308) | | |
| AHH03784 | QSLLNSSN | FAS (SEQ | QQHYSTPYT |
| | QKNY (SEQ | ID NO: | (SEQ ID NO: |
| | ID NO: | 957) | 1265) |
| | 1302) | | |
| AHH03785 | QDVSTA | SAS (SEQ | QQHYSTHV |
| | (SEQ ID NO: | ID NO: 13) | H (SEQ ID |
| | 1209) | | NO: 1259) |
| AHH03786 | QSLVHSNG | KVS (SEQ | SQSTHVPWT |
| | NTY (SEQ | ID NO: | (SEQ ID NO: |
| | ID NO: | 1062) | 1374) |
| | 1306) | | |
| AHH03787 | SSVIY (SEQ | DTS (SEQ | QQWTSNPPT |
| | ID NO: | ID NO: | (SEQ ID NO: |
| | 1383) | 839) | 1279) |
| AHH03788 | QNVGTN | SAS (SEQ | SQSTHVPYT |
| | (SEQ ID NO: | ID NO: 13) | (SEQ ID NO: |
| | 12) | | 1375) |
| AHH03790 | SRVTY | DTS (SEQ | HQRSGYSYT |
| | (SEQ ID NO: | ID NO: | (SEQ ID NO: |
| | 1380) | 839) | 1000) |
| AHH03791 | SRVTY | DTS (SEQ | HQRSGYSYT |
| | (SEQ ID NO: | ID NO: | (SEQ ID NO: |
| | 1380) | 839) | 1000) |
| AHH03792 | QNVGTN | SAS (SEQ | QQYNSYPRT |
| | (SEQ ID NO: | ID NO: 13) | (SEQ ID NO: |
| | 12) | | 1286) |
| AHH03793 | KSLLHSNG | RMS (SEQ | MQHLEYPY |
| | NTY (SEQ | ID NO: | T (SEQ ID |
| | ID NO: | 1360) | NO: 1136) |
| | 1060) | | |
| AHH03794 | QDISNY | YTS (SEQ | QQGNTLPW |
| | (SEQ ID NO: | ID NO: | T (SEQ ID |
| | 27) | 1495) | NO: 1255) |
| AHH03795 | QDISNY | YTS (SEQ | QQGNTLPW |
| | (SEQ ID NO: | ID NO: | T (SEQ ID |
| | 27) | 1495) | NO: 1255) |
| AHH03797 | QSLENSNG | RVS (SEQ | LQVTHVPFA |
| | NTY (SEQ | ID NO: | (SEQ ID NO: |
| | ID NO: | 1361) | 1096) |
| | 1300) | | |
| AHH03799 | QSLVHSYG | KVS (SEQ | SQSTHVPHT |
| | NTY (SEQ | ID NO: | (SEQ ID NO: |
| | ID NO: | 1062) | 1369) |
| | 1307) | | |
| >AH04501- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04515- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04502- | KASQNVGI | SASYRYS | QQYNSYPY |
| VH | NVA (SEQ | G (SEQ ID | T (SEQ ID |
| | ID NO: | NO: 1362) | NO: 15) |
| | 1037) | | |
| >AH04512- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04522- | KAGQNVG | SASYRYS | QQYNSYPY |
| VH | TNVA (SEQ | G (SEQ ID | T (SEQ ID |
| | ID NO: | NO: 1362) | NO: 15) |
| | 1036) | | |
| >AH04513- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04523- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04503- | KASQNVGT | SASYRYS | QQYNSYPW |
| VH | NVA (SEQ | G (SEQ ID | T (SEQ ID |
| | ID NO: | NO: 1362) | NO: 36) |
| | 1038) | | |
| >AH04529- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04510- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04528- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04511- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04504- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04520- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04507- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04527- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04505- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04516- | KASQNVGT | SASYRYS | QQYNSYPW |
| VH | NVA (SEQ | G (SEQ ID | T (SEQ ID |
| | ID NO: | NO: 1362) | NO: 36) |
| | 1038) | | |
| >AH04525- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04509- | KASQNVGT | SASYRYS | QQYNNSPLT |
| VH | NVA (SEQ | G (SEQ ID | (SEQ ID NO: |
| | ID NO: | NO: 1362) | 1282) |
| | 1038) | | |
| >AH04521- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04526- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04514- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04524- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04530- | KASQNVGT | SASYRYS | QQYNSYPW |
| VH | NVA (SEQ | G (SEQ ID | T (SEQ ID |
| | ID NO: | NO: 1362) | NO: 36) |
| | 1038) | | |
| >AH04517- | KASQSVGT | SASYRYS | QQYNNYPW |
| VH | NVA (SEQ | G (SEQ ID | T (SEQ ID |
| | ID NO: | NO: 1362) | NO: 1284) |
| | 1040) | | |
| >AH04506- | KASQNVGT | SASYRYS | QQYNSYPW |
| VH | NVA (SEQ | G (SEQ ID | T (SEQ ID |
| | ID NO: | NO: 1362) | NO: 36) |
| | 1038) | | |
| >AH04508- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04518- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| AH04734 | QDIRNY | YTS (SEQ | QQGNTLPPT |
| | (SEQ ID NO: | ID NO: | (SEQ ID NO: |
| | 1202) | 1495) | 1253) |
| AH04750 | QDISNY | YTS (SEQ | QQGNTLPPT |
| | (SEQ ID NO: | ID NO: | (SEQ ID NO: |
| | 27) | 1495) | 1253) |
| AH05214 | SSISSSN | GTS (SEQ | QQWSSYPLT |
| | (SEQ ID NO: | ID NO: | (SEQ ID NO: |
| | 1382) | 983) | 1277) |
| AH05247 | QNVGTN | SAS (SEQ | QQYNSYPLT |
| | (SEQ ID NO: | ID NO: 13) | (SEQ ID NO: |
| | 12) | | 42) |
| AH05249 | QSVNND | YAS (SEQ | QQDYRSPYT |
| | (SEQ ID NO: | ID NO: | (SEQ ID NO: |
| | 1309) | 1476) | 1249) |
| AH05251 | QSIVHSNG | KVS (SEQ | FQGSHVPPT |
| | NTY (SEQ | ID NO: | (SEQ ID NO: |
| | ID NO: | 1062) | 966) |
| | 1299) | | |
| AH05256 | QDISNY | HTS (SEQ | QQSNSLPPT |
| | (SEQ ID NO: | ID NO: | (SEQ ID NO: |
| | 27) | 1002) | 1271) |
| AH05257 | QDISNY | YTS (SEQ | QQGHTLPPT |
| | (SEQ ID NO: | ID NO: | (SEQ ID NO: |
| | 27) | 1495) | 1252) |
| AH05258 | QDVSTA | SAS (SEQ | QQHYSTPFT |
| | (SEQ ID NO: | ID NO: 13) | (SEQ ID NO: |
| | 1209) | | 1260) |
| AH05259 | ENIYSF | NAK (SEQ | QHHYGIPYT |
| | (SEQ ID NO: | ID NO: 28) | (SEQ ID NO: |
| | 928) | | 1214) |
| AH05268 | QDINRY | RAN (SEQ | LQYDEFPYT |
| | (SEQ ID NO: | ID NO: | (SEQ ID NO: |
| | 1201) | 1345) | 1106) |
| AH05271 | ENIYFS | NAN (SEQ | KQAYDVPW |
| | (SEQ ID NO: | ID NO: | T (SEQ ID |
| | 927) | 1142) | NO: 1058) |
| AH05274 | QNVGTN | SAS (SEQ | QQYNSYPY |
| | (SEQ ID NO: | ID NO: 13) | T (SEQ ID |
| | 12) | | NO: 15) |
| AH05280 | QSVNND | YAS (SEQ | QQAYWSPY |
| | (SEQ ID NO: | ID NO: | T (SEQ ID |
| | 1309) | 1476) | NO: 1248) |
| AH05285 | QDISNY | YTS (SEQ | QQGNTLPPT |
| | (SEQ ID NO: | ID NO: | (SEQ ID NO: |
| | 27) | 1495) | 1253) |
| AH05286 | QDVNTA | WAS (SEQ | QQHYSSPW |
| | (SEQ ID NO: | ID NO: | T (SEQ ID |
| | 1206) | 1450) | NO: 1258) |
| AH4501 | QNVGTN | SAS (SEQ | QQYNSYPY |
| | (SEQ ID NO: | ID NO: 13) | T (SEQ ID |
| | 12) | | NO: 15) |
| AH4502 | QNVGIN | SAS (SEQ | QQYNSYPY |
| | (SEQ ID NO: | ID NO: 13) | T (SEQ ID |
| | 1243) | | NO: 15) |
| AH4503 | QNVGTN | SAS (SEQ | QQYNSYPW |
| | (SEQ ID NO: | ID NO: 13) | T (SEQ ID |
| | 12) | | NO: 36) |
| AH4505 | QNVGTN | SAS (SEQ | QQYNSYPY |
| | (SEQ ID NO: | ID NO: 13) | T (SEQ ID |
| | 12) | | NO: 15) |
| AH4509 | QNVGTN | SAS (SEQ | QQYNNSPLT |
| | (SEQ ID NO: | ID NO: 13) | (SEQ ID NO: |
| | 12) | | 1282) |
| AH4511 | QNVGTN | SAS (SEQ | QQYNSYPY |
| | (SEQ ID NO: | ID NO: 13) | T (SEQ ID |
| | 12) | | NO: 15) |
| AH4518 | QNVGTN | SAS (SEQ | QQYNSYPY |
| | (SEQ ID NO: | ID NO: 13) | T (SEQ ID |
| | 12) | | NO: 15) |
| AH4523 | QNVGTN | SAS (SEQ | QQYNSYPY |
| | (SEQ ID NO: | ID NO: 13) | T (SEQ ID |
| | 12) | | NO: 15) |
| AH4524 | QNVGTN | SAS (SEQ | QQYNSYPY |
| | (SEQ ID NO: | ID NO: 13) | T (SEQ ID |
| | 12) | | NO: 15) |
| AH4525 | QNVGTN | SAS (SEQ | QQYNSYPY |
| | (SEQ ID NO: | ID NO: 13) | T (SEQ ID |
| | 12) | | NO: 15) |
| BP003- | QNVGTN | SAS (SEQ | QQYNSYPY |
| T2P1C4 | (SEQ ID NO: | ID NO: 13) | T (SEQ ID |
| | 12) | | NO: 15) |
| BP003- | QNVGTN | SAS (SEQ | QQYNRYPY |
| T2P1D10 | (SEQ ID NO: | ID NO: 13) | T (SEQ ID |
| | 12) | | NO: 1285) |
| BP003- | QSIVHSNG | KVS (SEQ | FQGSHVPPT |
| T2P1D7 | NTY (SEQ | ID NO: | (SEQ ID NO: |
| | ID NO: | 1062) | 966) |
| | 1299) | | |
| BP003- | QNVGTN | SAS (SEQ | QQYNSYPY |
| T2P1E3 | (SEQ ID NO: | ID NO: 13) | T (SEQ ID |
| | 12) | | NO: 15) |
| BP003- | ENSYSY | NAK (SEQ | QHHYGTPY |
| T2P1D1 | (SEQ ID NO: | ID NO: 28) | T (SEQ ID |
| | 52) | | NO: 54) |
| BL_592 | QNVGTN | SAS (SEQ | QQYNSYPY |
| 989- | (SEQ ID NO: | ID NO: 13) | T (SEQ ID |
| 2015_G2_ | 12) | | NO: 15) |
| P8_G02 | | | |
| BL_592 | QSLVHSNG | EVS (SEQ | SQSTHVPYT |
| 989- | NTY (SEQ | ID NO: | (SEQ ID NO: |
| 2016_H2_ | ID NO: | 944) | 1375) |
| P8_H02 | 1306) | | |
| BL_592 | QDVNTAV | WAS (SEQ | QQHYSSPW |
| 989- | A (SEQ ID | ID NO: | T (SEQ ID |
| 2017_A3_ | NO: 1207) | 1450) | NO: 1258) |
| P8_A03 | | | |
| BL_592 | QNVGTN | SAS (SEQ | QQYNSYPY |
| 989- | (SEQ ID NO: | ID NO: 13) | T (SEQ ID |
| 2018_B3_ | 12) | | NO: 15) |
| P8_B03 | | | |
| BL_592 | QNVGTN | SAS (SEQ | QQYNSYPY |
| 989- | (SEQ ID NO: | ID NO: 13) | T (SEQ ID |
| 2019_C3_ | 12) | | NO: 15) |
| P8_C03 | | | |
| BL_592 | QNVGTN | SAS (SEQ | QQYNSYPY |
| 989- | (SEQ ID NO: | ID NO: 13) | T (SEQ ID |
| 2021_E3_ | 12) | | NO: 15) |
| P8_E03 | | | |
| BL_592 | QNVGTN | SAS (SEQ | QQYNSYPY |
| 989- | (SEQ ID NO: | ID NO: 13) | T (SEQ ID |
| 2022_F3_ | 12) | | NO: 15) |
| P8_F03 | | | |
| BL_592 | QSLVHSNG | EVS (SEQ | SQSTHVPYT |
| 989- | NTY (SEQ | ID NO: | (SEQ ID NO: |
| 2023_G3_ | ID NO: | 944) | 1375) |
| P8_G03 | 1306) | | |
| BL_592 | QDIRNY | YTS (SEQ | QQGNTLPPT |
| 989- | (SEQ ID NO: | ID NO: | (SEQ ID NO: |
| 2024_H3_ | 1202) | 1495) | 1253) |
| P8_H03 | | | |
| BL_592 | QNVGTN | SAS (SEQ | QQYNSYPY |
| 989- | (SEQ ID NO: | ID NO: 13) | T (SEQ ID |
| 2025_A4_ | 12) | | NO: 15) |
| P8_A04 | | | |
| BL_592 | ENIYSY | NAK (SEQ | QHHYGSPY |
| 989- | (SEQ ID NO: | ID NO: 28) | T (SEQ ID |
| 2026_B4_ | 930) | | NO: 1216) |
| P8_B04 | | | |
| BL_592 | QNVGTN | SAS (SEQ | QQYNSYPY |
| 989- | (SEQ ID NO: | ID NO: 13) | T (SEQ ID |
| 2027_C4_ | 12) | | NO: 15) |
| P8_C04 | | | |
| BL_592 | QNVGTN | SAS (SEQ | QQYNSYPY |
| 989- | (SEQ ID NO: | ID NO: 13) | T (SEQ ID |
| 2029_E4_ | 12) | | NO: 15) |
| P8_E04 | | | |
| BL_592 | QNVGTN | SAS (SEQ | QQYNSYPW |
| 989- | (SEQ ID NO: | ID NO: 13) | T (SEQ ID |
| 2030_F4_ | 12) | | NO: 36) |
| P8_F04 | | | |
| BL_592 | QNVGTN | SAS (SEQ | QQYNSYPY |
| 989- | (SEQ ID NO: | ID NO: 13) | T (SEQ ID |
| 2031_G4_ | 12) | | NO: 15) |
| P8_G04 | | | |
| BL_592 | QNVGTN | SAS (SEQ | QQYNSYPY |
| 989- | (SEQ ID NO: | ID NO: 13) | T (SEQ ID |
| 2032_H4_ | 12) | | NO: 15) |
| P8_H04 | | | |
| BL_592 | ENIYSY | NAK (SEQ | QHHYGIPYT |
| 989- | (SEQ ID NO: | ID NO: 28) | (SEQ ID NO: |
| 2033_A5_ | 930) | | 1214) |
| P8_A05 | | | |
| BL_592 | QNVGTN | SAS (SEQ | QQYNSYPY |
| 989- | (SEQ ID NO: | ID NO: 13) | T (SEQ ID |
| 2034_B5_ | 12) | | NO: 15) |
| P8_B05 | | | |
| BL_592 | QNVGTN | SAS (SEQ | QQYNSYPY |
| 989- | (SEQ ID NO: | ID NO: 13) | T (SEQ ID |
| 2035_C5_ | 12) | | NO: 15) |
| P8_C05 | | | |
| BL_592 | QNVGTN | SAS (SEQ | QQYNSYPY |
| 989- | (SEQ ID NO: | ID NO: 13) | T (SEQ ID |
| 2037_E5_ | 12) | | NO: 15) |
| P8_E05 | | | |
| BL_592 | QNVGTN | SAS (SEQ | QQYNSYPY |
| 989- | (SEQ ID NO: | ID NO: 13) | T (SEQ ID |
| 2038_F5_ | 12) | | NO: 15) |
| P8_F05 | | | |
| BL_592 | QNVGTN | SAS (SEQ | QQYNNYPW |
| 989- | (SEQ ID NO: | ID NO: 13) | T (SEQ ID |
| 2039_G5_ | 12) | | NO: 1284) |
| P8_G05 | | | |
| BL_592 | QNVGTN | SAS (SEQ | QQYNSYPY |
| 989- | (SEQ ID NO: | ID NO: 13) | T (SEQ ID |
| 2040_H5_ | 12) | | NO: 15) |
| P8_H05 | | | |
| BL_592 | ENSYSY | NAK (SEQ | QHHYGTPY |
| 989- | (SEQ ID NO: | ID NO: 28) | T (SEQ ID |
| 2041_A6_ | 52) | | NO: 54) |
| P8_A06 | | | |
| BL_592 | ENSYSY | NAK (SEQ | QHHYGTPY |
| 989- | (SEQ ID NO: | ID NO: 28) | T (SEQ ID |
| 2042_B6_ | 52) | | NO: 54) |
| P8_B06 | | | |
| BL_592 | ENIYSY | NAK (SEQ | QHHYGTPY |
| 989- | (SEQ ID NO: | ID NO: 28) | T (SEQ ID |
| 2043_C6_ | 930) | | NO: 54) |
| P8_C06 | | | |
| BL_592 | QNVGTN | SAS (SEQ | QQYNTYPY |
| 989- | (SEQ ID NO: | ID NO: 13) | T (SEQ ID |
| 2044_D6_ | 12) | | NO: 1287) |
| P8_D06 | | | |
| BL_592 | QNVGTN | SAS (SEQ | QQYNSYPY |
| 989- | (SEQ ID NO: | ID NO: 13) | T (SEQ ID |
| 2045_E6_ | 12) | | NO: 15) |
| P8_E06 | | | |
| BL_592 | QNVGTN | SAS (SEQ | QQYNSYPY |
| 989- | (SEQ ID NO: | ID NO: 13) | T (SEQ ID |
| 2046_F6_ | 12) | | NO: 15) |
| P8_F06 | | | |
| BL_592 | QNVGTN | SAS (SEQ | QQYNNYPW |
| 989- | (SEQ ID NO: | ID NO: 13) | T (SEQ ID |
| 2047_G6_ | 12) | | NO: 1284) |
| P8_G06 | | | |
| BL_592 | QNVGTN | SAS (SEQ | QQYNSYPY |
| 989- | (SEQ ID NO: | ID NO: 13) | T (SEQ ID |
| 2048_H6_ | 12) | | NO: 15) |
| P8_H06 | | | |
| BL_592 | ENIYSY | NAK (SEQ | QHHYGTPY |
| 989- | (SEQ ID NO: | ID NO: 28) | T (SEQ ID |
| 2049_A7_ | 930) | | NO: 54) |
| P8_A07 | | | |
| BL_592 | QNVGTN | SAS (SEQ | QQYNSYPY |
| 989- | (SEQ ID NO: | ID NO: 13) | T (SEQ ID |
| 2051_C7_ | 12) | | NO: 15) |
| P8_C07 | | | |
| BL_592 | QSIVHSNG | KVS (SEQ | FQGSHVPPT |
| 989- | NTY (SEQ | ID NO: | (SEQ ID NO: |
| 2052_D7_ | ID NO: | 1062) | 966) |
| P8_D07 | 1299) | | |
| BL_592 | QNVGTN | SAS (SEQ | QQYNNHPY |
| 989- | (SEQ ID NO: | ID NO: 13) | T (SEQ ID |
| 2054_F7_ | 12) | | NO: 1281) |
| P8_F07 | | | |
| BL_592 | QNVGTN | SAS (SEQ | QQYNSYPW |
| 989- | (SEQ ID NO: | ID NO: 13) | T (SEQ ID |
| 2055_G7_ | 12) | | NO: 36) |
| P8_G07 | | | |
| BL_592 | QNVGTN | SAS (SEQ | QQYNTYPY |
| 989- | (SEQ ID NO: | ID NO: 13) | T (SEQ ID |
| 2059_C8_ | 12) | | NO: 1287) |
| P8_C08 | | | |
| BL_592 | ENIYSY | NAK (SEQ | QHHYGSPY |
| 989- | (SEQ ID NO: | ID NO: 28) | T (SEQ ID |
| 2060_D8_ | 930) | | NO: 1216) |
| P8_D08 | | | |
| BL_592 | QNVGTN | SAS (SEQ | QQYNSYPY |
| 989- | (SEQ ID NO: | ID NO: 13) | T (SEQ ID |
| 2061_E8_ | 12) | | NO: 15) |
| P8_E08 | | | |
| BL_592 | ENIYSF | NAK (SEQ | QHHYGTPY |
| 989- | (SEQ ID NO: | ID NO: 28) | T (SEQ ID |
| 2062_F8_ | 928) | | NO: 54) |
| P8_F08 | | | |
| BL_592 | QNVGTNV | SAS (SEQ | QQYNSYPY |
| 989- | A (SEQ ID | ID NO: 13) | T (SEQ ID |
| 2063_G8_ | NO: 1246) | | NO: 15) |
| P8_G08 | | | |
| BL_592 | QNVGTN | SAS (SEQ | QQYNSYPW |
| 989- | (SEQ ID NO: | ID NO: 13) | T (SEQ ID |
| 2064_H8_ | 12) | | NO: 36) |
| P8_H08 | | | |
| BL_592 | ENIYSF | NAK (SEQ | QHHYGTPY |
| 989- | (SEQ ID NO: | ID NO: 28) | T (SEQ ID |
| 2065_A9_ | 928) | | NO: 54) |
| P8_A09 | | | |
| BL_592 | QNVGTN | SAS (SEQ | QQYNNYPL |
| 989- | (SEQ ID NO: | ID NO: 13) | T (SEQ ID |
| 2067_C9_ | 12) | | NO: 1283) |
| P8_C09 | | | |
| BL_592 | ENIYSY | NAK (SEQ | QHHYDTPY |
| 989- | (SEQ ID NO: | ID NO: 28) | T (SEQ ID |
| 2068_D9_ | 930) | | NO: 30) |
| P8_D09 | | | |
| BL_592 | QNVGTN | SAS (SEQ | QQYNSYPY |
| 989- | (SEQ ID NO: | ID NO: 13) | T (SEQ ID |
| 2069_E9_ | 12) | | NO: 15) |
| P8_E09 | | | |
| BL_592 | ENIYSY | NAK (SEQ | QHHYGSPY |
| 989- | (SEQ ID NO: | ID NO: 28) | T (SEQ ID |
| 2070_F9_ | 930) | | NO: 1216) |
| P8_F09 | | | |
| BL_592 | QNVGTN | SAS (SEQ | QQYNSYPY |
| 989- | (SEQ ID NO: | ID NO: 13) | T (SEQ ID |
| 2071_G9_ | 12) | | NO: 15) |
| P8_G09 | | | |
| BL_592 | QNVGTN | SAS (SEQ | QQYNSYPY |
| 989- | (SEQ ID NO: | ID NO: 13) | T (SEQ ID |
| 2074__ | 12) | | NO: 15) |
| B10_ | | | |
| P8_B10 | | | |
| BL_592 | QNVGTN | SAS (SEQ | QQYNRYPY |
| 989- | (SEQ ID NO: | ID NO: 13) | T (SEQ ID |
| 2076_ | 12) | | NO: 1285) |
| D10_P8_ | | | |
| D10 | | | |
| BL_592 | QNVGTN | SAS (SEQ | QQYNSYPW |
| 989- | (SEQ ID NO: | ID NO: 13) | T (SEQ ID |
| 2077_ | 12) | | NO: 36) |
| E10_ | | | |
| P8_E10 | | | |
| BL_592 | QNVGTN | SAS (SEQ | QQYNSYPY |
| 989- | (SEQ ID NO: | ID NO: 13) | T (SEQ ID |
| 2078_ | 12) | | NO: 15) |
| F10_ | | | |
| P8_F10 | | | |
| BL_592 | QNVGTN | SAS (SEQ | QQYNSYPW |
| 989- | (SEQ ID NO: | ID NO: 13) | T (SEQ ID |
| 2079_ | 12) | | NO: 36) |
| G10_ | | | |
| P8_G10 | | | |
| BL_592 | QDIRNY | YTS (SEQ | QQGNTLPPT |
| 989- | (SEQ ID NO: | ID NO: | (SEQ ID NO: |
| 2080_ | 1202) | 1495) | 1253) |
| H10_ | | | |
| P8_H10 | | | |
| BP003- | ENIYSY | NAK (SEQ | QHHYGTPY |
| T2P1A7 | (SEQ ID NO: | ID NO: 28) | T (SEQ ID |
| | 930) | | NO: 54) |
| BP003- | QNVGTN | SAS (SEQ | QQYNTYPY |
| T2P1C8 | SEQ ID NO: | ID NO: 13) | T (SEQ ID |
| | 12) | | NO: 1287) |
| BP003- | QNVGTN | SAS (SEQ | QQYNSYPW |
| T2P1F4 | (SEQ ID NO: | ID NO: 13) | T (SEQ ID |
| | 12) | | NO: 36) |
| BP003- | QNVGTN | SAS (SEQ | QQYNSYPY |
| T2P1G2 | (SEQ ID NO: | ID NO: 13) | T (SEQ ID |
| | 12) | | NO: 15) |
| BP003- | QNVGTN | SAS (SEQ | QQYNSYPY |
| T2P1G4 | SEQ ID NO: | ID NO: 13) | T (SEQ ID |
| | 12) | | NO: 15) |
| BP003- | QNVGTN | SAS (SEQ | QQYNSYPW |
| T2P1G7 | (SEQ ID NO: | ID NO: 13) | T (SEQ ID |
| | 12) | | NO: 36) |
| BP003- | QNVGTN | SAS (SEQ | QQYNSYPY |
| T2P1H5 | (SEQ ID NO: | ID NO: 13) | T (SEQ ID |
| | 12) | | NO: 15) |
| BP003T | ENIYSY | NAK (SEQ | QHHYDTPY |
| 3P2-1 | (SEQ ID NO: | ID NO: 28) | T (SEQ ID |
| | 930) | | NO: 30) |
| BP003T | QDINRY | RAN (SEQ | LQYDEFPYT |
| 3P2-10 | (SEQ ID NO: | ID NO: | (SEQ ID NO: |
| | 1201) | 1345) | 1106) |
| BP003T | QDINRY | RAN (SEQ | LQYDEFPYT |
| 3P2-11 | (SEQ ID NO: | ID NO: | (SEQ ID NO: |
| | 1201) | 1345) | 1106) |
| BP003T | QDINNF | RAN (SEQ | LQYDEFPW |
| 3P2-12 | (SEQ ID NO: | ID NO: | T (SEQ ID |
| | 1200) | 1345) | NO: 1105) |
| BP003T | ENIYSY | NAK (SEQ | QHHYGSPY |
| 3P2-13 | (SEQ ID NO: | ID NO: 28) | T (SEQ ID |
| | 930) | | NO: 1216) |
| BP003T | ENIYSY | NAK (SEQ | QHHYDTPY |
| 3P2-14 | (SEQ ID NO: | ID NO: 28) | T (SEQ ID |
| | 930) | | NO: 30) |
| BP003T | ENSYSY | NAK (SEQ | QHHYGTPY |
| 3P2-15 | (SEQ ID NO: | ID NO: 28) | T (SEQ ID |
| | 52) | | NO: 54) |
| BP003T | ENIYSY | NAK (SEQ | QHHYDTPY |
| 3P2-16 | (SEQ ID NO: | ID NO: 28) | T (SEQ ID |
| | 930) | | NO: 30) |
| BP003T | ENIYSY | NAK (SEQ | QHHYDTPY |
| 3P2-17 | (SEQ ID NO: | ID NO: 28) | T (SEQ ID |
| | 930) | | NO: 30) |
| BP003T | ENIYSY | NAK (SEQ | QHHYGSPY |
| 3P2-18 | (SEQ ID NO: | ID NO: 28) | T (SEQ ID |
| | 930) | | NO: 1216) |
| BP003T | ENIYSY | NAK (SEQ | QHHYDTPY |
| 3P2-19 | (SEQ ID NO: | ID NO: 28) | T (SEQ ID |
| | 930) | | NO: 30) |
| BP003T | ENIYSY | NAK (SEQ | QHHYDTPY |
| 3P2-2 | (SEQ ID NO: | ID NO: 28) | T (SEQ ID |
| | 930) | | NO: 30) |
| BP003T | KNSYSY | NAK (SEQ | QHHYGTPY |
| 3P2-20 | (SEQ ID NO: | ID NO: 28) | T (SEQ ID |
| | 1057) | | NO: 54) |
| BP003T | ENIYSY | NAK (SEQ | QHHYGTPY |
| 3P2-21 | (SEQ ID NO: | ID NO: 28) | T (SEQ ID |
| | 930) | | NO: 54) |
| BP003T | ENSYSY | NAK (SEQ | QHHYGTPY |
| 3P2-22 | (SEQ ID NO: | ID NO: 28) | T (SEQ ID |
| | 52) | | NO: 54) |
| BP003T | ENSYSY | NAK (SEQ | QHHYGSPY |
| 3P2-23 | (SEQ ID NO: | ID NO: 28) | T (SEQ ID |
| | 52) | | NO: 1216) |
| BP003T | ENSYSY | NAK (SEQ | QHHYGSPY |
| 3P2-24 | (SEQ ID NO: | ID NO: 28) | T (SEQ ID |
| | 52) | | NO: 1216) |
| BP003T | ENSYSY | NAK (SEQ | QHHYGSPY |
| 3P2-25 | (SEQ ID NO: | ID NO: 28) | T (SEQ ID |
| | 52) | | NO: 1216) |
| BP003T | ENIYSY | NAK (SEQ | QHHYGSPY |
| 3P2-26 | (SEQ ID NO: | ID NO: 28) | T (SEQ ID |
| | 930) | | NO: 1216) |
| BP003T | QDISNY | YAS (SEQ | QQGNTLPW |
| 3P2-27 | (SEQ ID NO: | ID NO: | T (SEQ ID |
| | 27) | 1476) | NO: 1255) |
| BP003T | QDISNY | HTS (SEQ | QQSNTLPPT |
| 3P2-28 | (SEQ ID NO: | ID NO: | (SEQ ID NO: |
| | 27) | 1002) | 1272) |
| BP003T | QDISNY | HTS (SEQ | QQSNSLPPT |
| 3P2-29 | (SEQ ID NO: | ID NO: | (SEQ ID NO: |
| | 27) | 1002) | 1271) |
| BP003T | ENIYSY | NAK (SEQ | QHHYDTPY |
| 3P2-3 | (SEQ ID NO: | ID NO: 28) | T (SEQ ID |
| | 930) | | NO: 30) |
| BP003T | QDISNY | HTS (SEQ | QQSNSLPPT |
| 3P2-30 | (SEQ ID NO: | ID NO: | (SEQ ID NO: |
| | 27) | 1002) | 1271) |
| BP003T | QDISNY | HTS (SEQ | QQSNSLPPT |
| 3P2-31 | (SEQ ID NO: | ID NO: | (SEQ ID NO: |
| | 27) | 1002) | 1271) |
| BP003T | QDISNY | YTS (SEQ | QQGNTLPPT |
| 3P2-32 | (SEQ ID NO: | ID NO: | (SEQ ID NO: |
| | 27) | 1495) | 1253) |
| BP003T | QDISNY | HTS (SEQ | QQSNSLPPT |
| 3P2-33 | (SEQ ID NO: | ID NO: | (SEQ ID NO: |
| | 27) | 1002) | 1271) |
| BP003T | QDISNY | HTS (SEQ | QQSNSLPPT |
| 3P2-34 | (SEQ ID NO: | ID NO: | (SEQ ID NO: |
| | 27) | 1002) | 1271) |
| BP003T | QDISNY | HTS (SEQ | QQSNTLPPT |
| 3P2-35 | (SEQ ID NO: | ID NO: | SEQ ID NO: |
| | 27) | 1002) | 1272) |
| BP003T | QDISNY | HTS (SEQ | QQSNTLPPT |
| 3P2-36 | (SEQ ID NO: | ID NO: | (SEQ ID NO: |
| | 27) | 1002) | 1272) |
| BP003T | QDISNY | HTS (SEQ | QQGNTLPPT |
| 3P2-37 | (SEQ ID NO: | ID NO: | (SEQ ID NO: |
| | 27) | 1002) | 1253) |
| BP003T | QDISNY | YTS (SEQ | QQGNTLPPT |
| 3P2-38 | (SEQ ID NO: | ID NO: | (SEQ ID NO: |
| | 27) | 1495) | 1253) |
| BP003T | QDISNY | HTS (SEQ | QQGNTLPPT |
| 3P2-39 | (SEQ ID NO: | ID NO: | (SEQ ID NO: |
| | 27) | 1002) | 1253) |
| BP003T | ENIYSY | NAK (SEQ | QHHYDTPY |
| 3P2-4 | (SEQ ID NO: | ID NO: 28) | T (SEQ ID |
| | 930) | | NO: 30) |
| BP003T | QNVGTN | SAS (SEQ | QQYNSYPY |
| 3P2-40 | (SEQ ID NO: | ID NO: 13) | T (SEQ ID |
| | 12) | | NO: 15) |
| BP003T | QNVGTN | SAS (SEQ | QQYNSYPY |
| 3P2-41 | (SEQ ID NO: | ID NO: 13) | T (SEQ ID |
| | 12) | | NO: 15) |
| BP003T | QNVGTN | SAS (SEQ | QQYNSYPY |
| 3P2-42 | (SEQ ID NO: | ID NO: 13) | T (SEQ ID |
| | 12) | | NO: 15) |
| BP003T | QNVGTN | SAS (SEQ | QQYNTYPY |
| 3P2-43 | (SEQ ID NO: | ID NO: 13) | T (SEQ ID |
| | 12) | | NO: 1287) |
| BP003T | QNVGTN | SAS (SEQ | QQYNSYPY |
| 3P2-44 | (SEQ ID NO: | ID NO: 13) | T (SEQ ID |
| | 12) | | NO: 15) |
| BP003T | QNVGTN | SAS (SEQ | QQYNTYPY |
| 3P2-45 | (SEQ ID NO: | ID NO: 13) | T (SEQ ID |
| | 12) | | NO: 1287) |
| BP003T | QDIGLN | ATS (SEQ | LQYASSPFT |
| 3P2-46 | (SEQ ID NO: | ID NO: | (SEQ ID NO: |
| | 1198) | 618) | 1097) |
| BP003T | QNVGTN | SAS (SEQ | QQYNTYPY |
| 3P2-47 | (SEQ ID NO: | ID NO: 13) | T (SEQ ID |
| | 12) | | NO: 1287) |
| BP003T | QNVGTN | SAS (SEQ | QQYNSYPY |
| 3P2-48 | (SEQ ID NO: | ID NO: 13) | T (SEQ ID |
| | 12) | | NO: 15) |
| BP003T | QNVGTN | SAS (SEQ | QQYNSYPY |
| 3P2-49 | (SEQ ID NO: | ID NO: 13) | T (SEQ ID |
| | 12) | | NO: 15) |
| BP003T | ENIYSY | NAK (SEQ | QHHYDTPY |
| 3P2-5 | (SEQ ID NO: | ID NO: 28) | T (SEQ ID |
| | 930) | | NO: 30) |
| BP003T | QNVGTN | SAS (SEQ | QQYNSYPY |
| 3P2-50 | (SEQ ID NO: | ID NO: 13) | T (SEQ ID |
| | 12) | | NO: 15) |
| BP003T | QSLVHSNG | KVS (SEQ | SQSTHVPPT |
| 3P2-51 | NTY (SEQ | ID NO: | (SEQ ID NO: |
| | ID NO: | 1062) | 1371) |
| | 1306) | | |
| BP003T | QNVGTN | SAS (SEQ | QQYNSYPY |
| 3P2-52 | (SEQ ID NO: | ID NO: 13) | T (SEQ ID |
| | 12) | | NO: 15) |
| BP003T | QNVGTN | SAS (SEQ | QQYNSYPY |
| 3P2-53 | (SEQ ID NO: | ID NO: 13) | T (SEQ ID |
| | 12) | | NO: 15) |
| BP003T | QNVGTN | SAS (SEQ | QQYNSYPY |
| 3P2-54 | (SEQ ID NO: | ID NO: 13) | T (SEQ ID |
| | 12) | | NO: 15) |
| BP003T | QNVGTN | SAS (SEQ | QQYNSYPY |
| 3P2-55 | (SEQ ID NO: | ID NO: 13) | T (SEQ ID |
| | 12) | | NO: 15) |
| BP003T | QNVGTN | SAS (SEQ | QQYNSYPY |
| 3P2-56 | (SEQ ID NO: | ID NO: 13) | T (SEQ ID |
| | 12) | | NO: 15) |
| BP003T | QNVGTN | SAS (SEQ | QQYNSYPY |
| 3P2-57 | (SEQ ID NO: | ID NO: 13) | T (SEQ ID |
| | 12) | | NO: 15) |
| BP003T | QNVGTN | SAS (SEQ | QQYNSYPY |
| 3P2-58 | (SEQ ID NO: | ID NO: 13) | T (SEQ ID |
| | 12) | | NO: 15) |
| BP003T | QDVRTA | STS (SEQ | QQYSNYLTF |
| 3P2-59 | (SEQ ID NO: | ID NO: | (SEQ ID NO: |
| | 1208) | 1386) | 1290) |
| BP003T | ENIYSY | NAK (SEQ | QHHYDTPY |
| 3P2-6 | (SEQ ID NO: | ID NO: 28) | T (SEQ ID |
| | 930) | | NO: 30) |
| BP003T | QNVGTN | SAS (SEQ | QQYNTYPY |
| 3P2-60 | (SEQ ID NO: | ID NO: 13) | T (SEQ ID |
| | 12) | | NO: 1287) |
| BP003T | QNVGTN | SAS (SEQ | XQYNSYPY |
| 3P2-61 | (SEQ ID NO: | ID NO: 13) | T (SEQ ID |
| | 12) | | NO: 1475) |
| BP003T | QSVNND | YAS (SEQ | QQAYWSPY |
| 3P2-62 | (SEQ ID NO: | ID NO: | T (SEQ ID |
| | 1309) | 1476) | NO: 1248) |
| BP003T | QDVNTA | WAS (SEQ | QQHYSSPW |
| 3P2-63 | (SEQ ID NO: | ID NO: | T (SEQ ID |
| | 1206) | 1450) | NO: 1258) |
| BP003T | QDVNTA | WAS (SEQ | QQHYSSPW |
| 3P2-64 | (SEQ ID NO: | ID NO: | T (SEQ ID |
| | 1206) | 1450) | NO: 1258) |
| BP003T | QDVNTA | WAS (SEQ | QQHYSSPW |
| 3P2-65 | (SEQ ID NO: | ID NO: | T (SEQ ID |
| | 1206) | 1450) | NO: 1258) |
| BP003T | QEISGY | AAS (SEQ | LQYASYPYT |
| 3P2-66 | (SEQ ID NO: | ID NO: | (SEQ ID NO: |
| | 1210) | 599) | 1103) |
| BP003T | QSIVHSNG | KVS (SEQ | FQGSHVPPT |
| 3P2-67 | NTY (SEQ | ID NO: | (SEQ ID NO: |
| | ID NO: | 1062) | 966) |
| | 1299) | | |
| BP003T | QSIVHSNG | KVS (SEQ | FQGSHVPRT |
| 3P2-68 | NTY (SEQ | ID NO: | (SEQ ID NO: |
| | ID NO: | 1062) | 967) |
| | 1299) | | |
| BP003T | QSIVHNNG | KVS (SEQ | FQGSYVPRT |
| 3P2-69 | NTY (SEQ | ID NO: | (SEQ ID NO: |
| | ID NO: | 1062) | 971) |
| | 1298) | | |
| BP003T | ENIYSY | NAK (SEQ | QHHYDTPY |
| 3P2-7 | (SEQ ID NO: | ID NO: 28) | T (SEQ ID |
| | 930) | | NO: 30) |
| BP003T | QSIVHSNG | KVS (SEQ | FQGSHVPPT |
| 3P2-70 | NTY (SEQ | ID NO: | (SEQ ID NO: |
| | ID NO: | 1062) | 966) |
| | 1299) | | |
| BP003T | QSIVHSNG | KVS (SEQ | FQGSHVPPT |
| 3P2-71 | NTY (SEQ | ID NO: | (SEQ ID NO: |
| | ID NO: | 1062) | 966) |
| | 1299) | | |
| BP003T | QSIVHSNG | KVS (SEQ | FQGSHVPTF |
| 3P2-72 | NTY (SEQ | ID NO: | (SEQ ID NO: |
| | ID NO: | 1062) | 968) |
| | 1299) | | |
| BP003T | QSIVHSNG | KVS (SEQ | FQGSHVPPT |
| 3P2-73 | NTY (SEQ | ID NO: | (SEQ ID NO: |
| | ID NO: | 1062) | 966) |
| | 1299) | | |
| BP003T | QSIVHSNG | KVS (SEQ | FQGSHVPPT |
| 3P2-74 | NTY (SEQ | ID NO: | (SEQ ID NO: |
| | ID NO: | 1062) | 966) |
| | 1299) | | |
| BP003T | QDVSTA | SAS (SEQ | QQHYSTPYT |
| 3P2-75 | (SEQ ID NO: | ID NO: 13) | (SEQ ID NO: |
| | 1209) | | 1265) |
| BP003T | ENIYSY | NAK (SEQ | QHHYDTPY |
| 3P2-8 | (SEQ ID NO: | ID NO: 28) | T (SEQ ID |
| | 930) | | NO: 30) |
| BP003T | DNIYNY | NAK (SEQ | QHHYGSPW |
| 3P2-9 | (SEQ ID NO: | ID NO: 28) | T (SEQ ID |
| | 833) | | NO: 1215). |

2. The method of claim 1, wherein the antibody depletes regulatory T cells in a subject.

3. The method of claim 1, wherein the subject suffers from cancer or an autoimmune-related disease or disorder.

4. The method of claim 1, wherein the subject suffers from an autoimmune-related disease or disorder.

5. The method of claim 2, wherein depleting the number of regulatory T cells in a sample comprises contacting peripheral blood mononuclear cells with the antibody.

6. The method of claim 1, wherein the antibody does not disrupt the binding of the IL-2 ligand to the alpha chain of the IL-2 receptor (CD25), and binds to a different epitope than a 7G7B6 antibody.

7. The method of claim 1, wherein the antibody does not disrupt the binding of the IL-2 ligand to the alpha chain of the IL-2 receptor (CD25), but does disrupt the trimerization of the beta, gamma, and alpha (CD25) chains of the IL-2 receptor.

8. The method of claim 1, wherein the antibody disrupts the binding of the IL-2 ligand to the IL-2 receptor, and binds to a different epitope than a Daclizumab or a Baciliximab antibody.

9. The method of claim 1, wherein the antibody exhibits a higher affinity of binding to CD25 at a pH lower than 7.4, when compared to the affinity of binding to CD25 at a pH of 7.4.

10. The method of claim 1, wherein the antibody exhibits a higher affinity of binding to CD25 at a pH of about 6.5.

11. The method of claim 1, wherein the antibody comprises:
at least a 80%, at least a 85%, at least a 90%, or at least a 95% sequence identity to the amino acid sequence of any one of the variable heavy chain sequences of SEQ ID NOS: 2, 4, 6, 8, 17, 19, 21, 23, 32, 38, 44, 46, 48, 56, 60-107, 114, 157, 190, 195, 230, 235, 251, 265, 269, 398, 413, 421, 422, 424, 425, 431-460, 473, 601, 603-606, 608, 611, 615, 617, 619, 868, 869, 936-940, 942, 943, 945-952, 954, 955, 973, 977, 984, 986, 987, 992, 995, 997, 1003-1008, 1010-1015, 1017, 1018, 1020, 1021, 1023, 1025-1027, 1029-1035, 1074, 1075, 1118, 1119, 1123-1128, 1137-1139, 1165-1172, 1174-1179, 1181-1185, 1187, 1188, 1190-1197, 1218, 1220, 1222, 1223-1241, 1313-1318, 1321-1343, 1387-1389, 1395, 1416, 1417, 1419, 1420, 1442-1445, 1453-1455, 1448, 1474, 1496; and
at least a 80%, at least a 85%, at least a 90%, or at least a 95% sequence identity to the amino acid sequence of any one of the variable light chain of SEQ ID NOS: 10, 12, 13, 15, 25, 27, 28, 30, 34, 36, 40, 42, 50, 52, 54, 59, 599, 618, 700, 701, 703, 705, 707, 709, 711, 712, 713, 715, 717-720, 722, 724, 725, 727-732, 734-740, 742, 744, 745, 747, 749-752, 754-757, 759, 760, 762, 765-767, 769, 770, 773, 775-778, 780, 781, 783, 785, 787, 789, 791, 793, 794, 795, 797, 799, 801, 803, 804, 806, 807, 809-811, 813, 815, 816, 818, 821-825, 827-833, 835, 837, 838, 841, 842, 844, 846, 848, 849, 851, 853, 854, 857, 858-861, 864, 865, 870-880, 882, 884, 886, 888, 889, 890, 892, 894, 895, 897, 899, 900-903, 905-909, 912, 914, 915, 917-922, 926-928, 930, 944, 958, 959, 961, 963, 966-968, 971, 974, 978, 983, 1002, 1057, 1058, 1062, 1070-1072, 1076, 1085, 1088, 1091, 1097, 1103, 1105, 1106, 1109, 1110, 1112, 1117, 1132, 1142, 1151, 1154, 1157-1159, 1164, 1198, 1200-1202, 1206-1210, 1214-1216, 1243, 1246, 1248, 1249, 1252, 1253, 1255, 1258, 1260, 1265, 1271, 1272, 1277, 1281-1285, 1287, 1290, 1298, 1299, 1306, 1309, 1345, 1347-1350, 1352, 1353, 1355-1357, 1359, 1366, 1371, 1375, 1382, 1386, 1396-1402, 1404, 1408, 1413, 1414, 1423-1425, 1427, 1429, 1430, 1432, 1433, 1436, 1447, 1449, 1450, 1470, 1471, 1475-1478, 1481-1483, 1486-1491, 1495, humanized versions thereof, or an amino acid sequence comprising at least a 80%, at least a 85%, at least a 90%, or at least a 95% sequence identity thereto.

12. The method of claim 1, wherein the CD25 antibody comprises:
a heavy chain of SEQ ID NOS: 4, 6, 8, 19, 21, 23, 46, 48, 151-183, 224-258, 297-344, 482-492, 506-515, 531-557, 601-608, 611, 612, 614, 615, 617, 619, 691-697, 973, 977, 982, 984-987, 989-997, 1003, 1004, 1012-1016, 1020-1029, 1035, 1165, 1377-1379, 1395, 1416-1420, 1442-1445; and
a light chain of SEQ ID NOS: 12, 13, 15, 27, 28, 30, 36, 42, 52, 54, 599, 600, 618, 833, 839, 924, 925, 927-930, 932-935, 944, 957, 966-972, 975, 976, 979-981, 983, 998-1002, 1036-1038, 1040, 1057-1063, 1081, 1092-1107, 1115, 1116, 1122, 1136, 1142, 1198-1202, 1204-1217, 1242-1244, 1246-1287, 1289-1296, 1298-1311, 1344-1346, 1360-1362, 1365, 1369-1376, 1380-1386, 1391, 1450, 1461-1466, 1475, 1476, 1495.

13. The method of claim 1, wherein the antibody is a human antibody.

14. The method of claim 1, wherein the antibody is a humanized antibody.

15. The method of claim, wherein the antibody is a chimeric antibody.

16. The method of claim 1, wherein the antibody comprises a mouse variable domain, and a human constant domain.

17. The method of claim 1, wherein the antibody also binds cynomologous monkey CD25.

18. The method of claim 17, wherein the antibody is delivered as a composition comprising a nucleic acid sequence or nucleic acids encoding the antibody of claim 1.

19. A method of treating a subject in need thereof comprising:
administering to the subject a therapeutically effective amount of a monoclonal antibody or binding fragment thereof that binds to human CD25 and depletes CD25+ cells, comprising:

| ID | CDRH1 | CDRH2 | CDRH3 |
|---|---|---|---|
| D11 | GYTFTDYA | ISTYSGDA | ARGVTFDY |
| | (SEQ ID NO: | (SEQ ID NO: | (SEQ ID NO: |
| | 19) | 21) | 23) |
| D17 | GYTFTNYW | IYPGGGYT | ARVTPAS |
| | (SEQ ID NO: | (SEQ ID NO: | (SEQ ID NO: |
| | 4) | 6) | 8) |
| D34 | GYTFTDYA | ISTYSGDV | ARGVTFDS |
| | (SEQ ID NO: | (SEQ ID NO: | (SEQ ID NO: |
| | 19) | 46) | 48) |
| D36 | GYTFTDYA | ISTYSGDA | ARGVTFDY |
| | (SEQ ID NO: | (SEQ ID NO: | (SEQ ID NO: |
| | 19) | 21) | 23) |
| D5 | GYTFTNYW | IYPGGGYT | ARVTPAS |
| | (SEQ ID NO: | (SEQ ID NO: | (SEQ ID NO: |
| | 4) | 6) | 8) |
| AHH03702 | GFTFSSYA | ISSGGST | ARGEIWGKA |
| | (SEQ ID NO: | (SEQ ID NO: | WFAY (SEQ |
| | 151) | 224) | ID NO: 297) |
| AHH03703 | GYTLTDYS | INTETGEP | AWGNHY |
| | (SEQ ID NO: | (SEQ ID NO: | (SEQ ID NO: |
| | 152) | 225) | 298) |
| AHH03704 | GYTFTSYV | INPYNDGT | ARDGYYVG |
| | (SEQ ID NO: | (SEQ ID NO: | PAY (SEQ ID |
| | 153) | 226) | NO: 299) |
| AHH03705 | GFAFSSYD | ISSGGGST | ARNYRSWF |
| | (SEQ ID NO: | (SEQ ID NO: | AY (SEQ ID |
| | 154) | 227) | NO: 300) |
| AHH03706 | GYAFTNYL | INPGSGGT | ARKGSLTGV |
| | (SEQ ID NO: | (SEQ ID NO: | LAY (SEQ ID |
| | 155) | 228) | NO: 301) |
| AHH03707 | GYIFTNYW | IDPSDSET | ARRGLRAW |
| | (SEQ ID NO: | (SEQ ID NO: | FAY (SEQ ID |
| | 156) | 229) | NO: 302) |
| AHH03708 | GYTFTSYW | INPSTGYT | ARLDYYGSS |
| | (SEQ ID NO: | (SEQ ID NO: | RGFAY (SEQ |
| | 157) | 230) | ID NO: 303) |
| AHH03709 | GYTFTSYY | INPSNGGT | TNGGGWY |
| | (SEQ ID NO: | (SEQ ID NO: | (SEQ ID NO: |
| | 158) | 231) | 304) |
| AHH03711 | GYTFTNYG | INTYTGEP | ASYYDSTYV |
| | (SEQ ID NO: | (SEQ ID NO: | GFAY (SEQ |
| | 159) | 232) | ID NO: 305) |
| AHH03712 | GFTFSDYY | ISNGGGST | ASPLGYDGF |
| | (SEQ ID NO: | (SEQ ID NO: | AY (SEQ ID |
| | 160) | 233) | NO: 306) |
| AHH03713 | GFSLTSYG | IWAGGST | ARGAYFDY |
| | (SEQ ID NO: | (SEQ ID NO: | (SEQ ID NO: |
| | 161) | 234) | 307) |
| AHH03714 | GYTFTSYW | IDPYDSET | ARSPAYYGN |
| | (SEQ ID NO: | (SEQ ID NO: | LWFAY (SEQ |
| | 157) | 235) | ID NO: 308) |
| AHH03715 | GYTFTSYT | INPSSGYT | ARWDGAY |
| | (SEQ ID NO: | (SEQ ID NO: | (SEQ ID NO: |
| | 162) | 236) | 309) |
| AHH03716 | GFTFSSYA | ISSGGSYT | ARGGMITPF |
| | (SEQ ID NO: | (SEQ ID NO: | AY (SEQ ID |
| | 151) | 237) | NO: 310) |
| AHH03717 | GFSLSTSGM | IWWNDDK | ARIGGNDGY |
| | S (SEQ ID | (SEQ ID NO: | YWYFDV |
| | NO: 163) | 238) | (SEQ ID NO: |
| | | | 311) |
| AHH03718 | GFTFSDAW | IRSKANNH | TPQFAY |
| | (SEQ ID NO: | AT (SEQ ID | (SEQ ID NO: |
| | 164) | NO: 239) | 312) |
| AHH03719 | GFTFSSYG | INSNGGST | ASHYDEGY |
| | (SEQ ID NO: | (SEQ ID NO: | (SEQ ID NO: |
| | 165) | 240) | 313) |
| AHH03720 | GFTFSNYW | IRLKSNNYA | TGSDY (SEQ |
| | (SEQ ID NO: | T (SEQ ID | ID NO: 314) |
| | 166) | NO: 241) | |
| AHH03721 | GDTFSSYV | FNPYSDDI | GSGYDGYY |
| | (SEQ ID NO: | (SEQ ID NO: | DWFAC (SEQ |
| | 167) | 242) | ID NO: 315) |
| AHH03722 | GYSFTKNG | INTYTGEP | AREPKTLDY |
| | (SEQ ID NO: | (SEQ ID NO: | (SEQ ID NO: |
| | 168) | 232) | 316) |
| AHH03724 | GYTFTSYW | IDPSDSET | ANWAWFAY |
| | (SEQ ID NO: | (SEQ ID NO: | (SEQ ID NO: |
| | 157) | 229) | 317) |
| AHH03726 | GYTFNSHW | IDPYDSET | ARPYDYDGF |
| | (SEQ ID NO: | (SEQ ID NO: | AY (SEQ ID |
| | 169) | 235) | NO: 318) |
| AHH03727 | GYTFTNHH | INPYNDYT | ADGDYYFD |
| | (SEQ ID NO: | (SEQ ID NO: | Y (SEQ ID |
| | 170) | 243) | NO: 319) |
| AHH03728 | GLTFSSYG | ISSGGSYI | ARQDDGYY |
| | (SEQ ID NO: | (SEQ ID NO: | RIFDY (SEQ |
| | 171) | 244) | ID NO: 320) |
| AHH03729 | GFTFNDAW | IRSKANNH | TNYGSNPLD |
| | (SEQ ID NO: | AT (SEQ ID | Y (SEQ ID |
| | 172) | NO: 239) | NO: 321) |
| AHH03730 | GFSLTSYG | IWAGGST | AREGTGPWF |
| | (SEQ ID NO: | (SEQ ID NO: | AY (SEQ ID |
| | 161) | 234) | NO: 322) |
| AHH03731 | GYSITSGYS | IHYSGST | ARDPPFAY |
| | (SEQ ID NO: | (SEQ ID NO: | (SEQ ID NO: |
| | 173) | 245) | 323) |
| AHH03733 | GYTFTSYW | IDPSDSYT | AREEITAWF |
| | (SEQ ID NO: | (SEQ ID NO: | AY (SEQ ID |
| | 157) | 246) | NO: 324) |
| AHH03734 | GYTFTDYE | IHPGSGGT | TRNGNGNW |
| | (SEQ ID NO: | (SEQ ID NO: | YFDV (SEQ |
| | 174) | 247) | ID NO: 325) |
| AHH03737 | GYAFSSYW | IYPGDGDT | ARSGYRYDA |
| | (SEQ ID NO: | (SEQ ID NO: | VFAY (SEQ |
| | 175) | 248) | ID NO: 326) |
| AHH03738 | GFTFSSYG | INSNGGST | ARGGNPY |
| | (SEQ ID NO: | (SEQ ID NO: | (SEQ ID NO: |
| | 165) | 240) | 327) |
| AHH03739 | GFTFSGFW | INSDGSAI | MRYGSSYW |
| | (SEQ ID NO: | (SEQ ID NO: | YFD (SEQ ID |
| | 176) | 249) | NO: 328) |
| AHH03740 | GFTFSSYW | IRLKSDNYA | TRYYYGES |
| | (SEQ ID NO: | T (SEQ ID | (SEQ ID NO: |
| | 177) | NO: 250) | 329) |
| AHH03742 | GFTFSSYG | ISSGGSYT | ARHYYDYD |
| | (SEQ ID NO: | (SEQ ID NO: | YWYFDV |
| | 165) | 237) | (SEQ ID NO: |
| | | | 330) |
| AHH03743 | GYTFTSYW | IYPGSGST | TRSGVEGLL |
| | (SEQ ID NO: | (SEQ ID NO: | HWYFDV |
| | 157) | 251) | (SEQ ID NO: |
| | | | 331) |
| AHH03746 | GFTFSSYW | IRLKSDNYA | TCDYDGGA |
| | (SEQ ID NO: | T (SEQ ID | WFAY (SEQ |
| | 177) | NO: 250) | ID NO: 332) |
| AHH03748 | GYTFTSYW | IDPSNSET | ARCDGYYD |
| | (SEQ ID NO: | (SEQ ID NO: | GLDY (SEQ |
| | 157) | 252) | ID NO: 333) |
| AHH03749 | GYTFTSYT | INPSSGYT | AREGKNWY |
| | (SEQ ID NO: | (SEQ ID NO: | FDV (SEQ ID |
| | 162) | 236) | NO: 334) |
| AHH03750 | GYTFTSYW | INPSNGGT | ARRIYRTLD |
| | (SEQ ID NO: | (SEQ ID NO: | Y (SEQ ID |
| | 157) | 231) | NO: 335) |
| AHH03751 | GYTFTSYN | IYPGNGDT | TRSGGNLWF |
| | (SEQ ID NO: | (SEQ ID NO: | AY (SEQ ID |
| | 178) | 253) | NO: 336) |
| AHH03752 | GYTFSSYW | ILPGSGST | ARRTYYGN |
| | (SEQ ID NO: | (SEQ ID NO: | AWFAY (SEQ |
| | 179) | 254) | ID NO: 337) |
| AHH03753 | GFTFTDYY | IRNKANGY | ARDKRITTV |
| | (SEQ ID NO: | TT (SEQ ID | EAWFAY |
| | 180) | NO: 255) | (SEQ ID NO: |
| | | | 338) |
| AHH03754 | GFTFSSYA | ISSGGST | ARGYGSSFA |
| | (SEQ ID NO: | (SEQ ID NO: | Y (SEQ ID |
| | 151) | 224) | NO: 339) |
| AHH03755 | GYTFTSYY | INPSNSGT | TRGGDYDAS |
| | (SEQ ID NO: | (SEQ ID NO: | WFAY (SEQ |
| | 158) | 256) | ID NO: 340) |
| AHH03756 | GYTLTDYV | IYPGSGST | ARRTARAFD |
| | (SEQ ID NO: | (SEQ ID NO: | Y (SEQ ID |
| | 181) | 251) | NO: 341) |
| AHH03757 | GFTFSSYA | ISSGGSYT | ARRIGYDGG |
| | (SEQ ID NO: | (SEQ ID NO: | GSWFAY |
| | 151) | 237) | (SEQ ID NO: |
| | | | 342) |
| AHH03758 | GFTFNT | IRSKNNY | CERVRRCV |
| | YA (SEQ | VT (SEQ ID | (SEQ ID NO: |
| | ID NO: 182) | NO: 257) | 343) |
| AHH03759 | GFTFSN | ITVKSDNY | SRWFAY |
| | YR (SEQ ID | GA (SEQ ID | (SEQ ID |
| | NO: 183) | NO: 258) | NO: 344) |
| AHH03760 | GYTFTDYE | IHPGSGGT | TRSDYGSSY |
| | (SEQ ID NO: | (SEQ ID NO: | EFAY (SEQ |
| | 174) | 247) | ID NO: 531) |
| AHH03765 | GYSITSDYA | ISYSGST | ARSRGNYFD |
| | (SEQ ID NO: | (SEQ ID NO: | Y (SEQ ID |
| | 482) | 506) | NO: 532) |
| AHH03767 | GYTFTSYT | INPSSGYT | ARSGLRQA |
| | (SEQ ID NO: | (SEQ ID NO: | WFAY (SEQ |
| | 162) | 236) | ID NO: 533) |
| AHH03768 | GFTFSNYR | ITVKSDNY | SRLFAY |
| | (SEQ ID NO: | GA (SEQ ID | (SEQ ID NO: |
| | 183) | NO: 258) | 534) |
| AHH03770 | GYTFTSYW | INPSNGGT | TITGFDV |
| | (SEQ ID NO: | (SEQ ID NO: | (SEQ ID NO: |
| | 157) | 231) | 535) |
| AHH03771 | GYTFTTYW | IFPGTGTT | ARGGYYNSS |
| | (SEQ ID NO: | (SEQ ID NO: | PFAY (SEQ |
| | 483) | 507) | ID NO: 536) |
| AHH03772 | GFTFTDYY | IRNKANGY | ARDGEVRRA |
| | (SEQ ID NO: | TT (SEQ ID | LAY (SEQ ID |
| | 180) | NO: 255) | NO: 537) |
| AHH03773 | GYTFTSST | INPSSGYT | VRHYYFDY |
| | (SEQ ID NO: | (SEQ ID NO: | (SEQ ID NO: |
| | 484) | 236) | 538) |
| AHH03774 | GFTFSSYT | ISSGGGYT | TRVSAKYFD |
| | (SEQ ID NO: | (SEQ ID NO: | V (SEQ ID |
| | 485) | 508) | NO: 539) |
| AHH03776 | GYTFTNFY | INPSNGGT | TRSYYDYD |
| | (SEQ ID NO: | (SEQ ID NO: | WYFDV (SEQ |
| | 486) | 231) | ID NO: 540) |
| AHH03777 | GYTFTSYW | IYPSDSYT | TRQNYYGSS |
| | (SEQ ID NO: | (SEQ ID NO: | HWYFDV |
| | 157) | 509) | (SEQ ID NO: |
| | | | 541) |
| AHH03778 | GYTFTSYW | IDPSDSET | ANWAWFAY |
| | (SEQ ID NO: | (SEQ ID NO: | (SEQ ID NO: |
| | 157) | 229) | 317) |
| AHH03779 | GYTFTDYS | INTETGEP | ASFYYGNFA |
| | (SEQ ID NO: | (SEQ ID NO: | YYFDY (SEQ |
| | 487) | 225) | ID NO: 542) |
| AHH03780 | GFTFSNYW | IRLKSNNYA | TRIYDSGSSY |
| | (SEQ ID NO: | T (SEQ ID | TWYFDV |
| | 166) | NO: 241) | (SEQ ID NO: |
| | | | 543) |
| AHH03782 | GFSLSTSGM | IWWNDDK | ARIGGNDGY |
| | S (SEQ ID | (SEQ ID NO: | YWYFDV |
| | NO: 163) | 238) | (SEQ ID NO: |
| | | | 311) |
| AHH03783 | GYTFTSYW | INPSNGRT | ARDSSGYGA |
| | (SEQ ID NO: | (SEQ ID NO: | Y (SEQ ID |
| | 157) | 510) | NO: 544) |
| AHH03784 | GYTFTSYW | INPSTGYT | ARYDGYYY |
| | (SEQ ID NO: | (SEQ ID NO: | FDY (SEQ ID |
| | 157) | 230) | NO: 545) |
| AHH03785 | GYTFTSYW | IYPSDSYT | TSHYYGRA |
| | (SEQ ID NO: | (SEQ ID NO: | WFAY (SEQ |
| | 157) | 509) | ID NO: 546) |
| AHH03786 | GYTFTRYY | INPSNGGT | TKGGFYDFF |
| | (SEQ ID NO: | (SEQ ID NO: | AY (SEQ ID |
| | 488) | 231) | NO: 547) |
| AHH03787 | GFTFSSYG | INSNGGST | ASLAY (SEQ |
| | (SEQ ID NO: | (SEQ ID NO: | ID NO: 548) |
| | 165) | 240) | |
| AHH03788 | GYTFTDYY | IYPGSGNT | ARVYSGFDV |
| | (SEQ ID NO: | (SEQ ID NO: | (SEQ ID NO: |
| | 489) | 511) | 549) |
| AHH03790 | GYTFTSYY | IYPGDGST | ARGDGYFA |
| | (SEQ ID NO: | (SEQ ID NO: | WFAY (SEQ |
| | 158) | 512) | ID NO: 550) |
| AHH03791 | GYTFSSYW | ILPGSGST | ARSAHRYDA |
| | (SEQ ID NO: | (SEQ ID NO: | WFAY (SEQ |
| | 179) | 254) | ID NO: 551) |
| AHH03792 | GYTFTNYY | INPTNGGT | TRGMAYRY |
| | (SEQ ID NO: | (SEQ ID NO: | DGAGWFAY |
| | 490) | 513) | (SEQ ID NO: |
| | | | 552) |
| AHH03793 | GYTFTSYW | IDPYDSET | ARGGRGTW |
| | (SEQ ID NO: | (SEQ ID NO: | FAY (SEQ ID |
| | 157) | 235) | NO: 553) |
| AHH03794 | GFTFTDYY | IRNKANGY | ARGWGNWF |
| | (SEQ ID NO: | TT (SEQ ID | AY (SEQ ID |
| | 180) | NO: 255) | NO: 554) |
| AHH03795 | GFTFSSFG | ISGGGGTI | ARWRGGYF |
| | (SEQ ID NO: | (SEQ ID NO: | DY (SEQ ID |
| | 491) | 514) | NO: 555) |
| AHH03797 | GYTFTSYW | IYPSDSYT | TRTGGSTMT |
| | (SEQ ID NO: | (SEQ ID NO: | PWFAY (SEQ |
| | 157) | 509) | ID NO: 556) |
| AHH03799 | GFSLSTSGM | IYWDDDK | ARRAGDYG |
| | G (SEQ ID | (SEQ ID NO: | NPFPY (SEQ |
| | NO: 492) | 515) | ID NO: 557) |
| >AH04501- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04515- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04502- | GYTFTNY | DIHPGGGY | SRNFAY |
| VH | (SEQ ID NO: | TNYNEKFK | (SEQ ID NO: |
| | 990) | G (SEQ ID | 1379) |
| | | NO: 696) | |
| >AH04512- | GYTFTNY | DIHPGGGY | SRNFAY |
| VH | (SEQ ID NO: | TNYNEKFK | (SEQ ID NO: |
| | 990) | G (SEQ ID | 1379) |
| | | NO: 696) | |
| >AH04522- | GYTFTNY | DIHPGGGY | SRNFAY |
| VH | (SEQ ID NO: | TNYNEKFK | (SEQ ID NO: |
| | 990) | G (SEQ ID | 1379) |
| | | NO: 696) | |
| >AH04513- | GYTFTNY | DIHPGGGY | SRNFAY |
| VH | (SEQ ID NO: | TNYNEKFK | (SEQ ID NO: |
| | 990) | G (SEQ ID | 1379) |
| | | NO: 696) | |
| >AH04523- | GYTFTNY | DIHPGGSY | SRNFAK |
| VH | (SEQ ID NO: | TNYNENFK | (SEQ ID NO: |
| | 990) | G (SEQ ID | 1377) |
| | | NO: 697) | |
| >AH04503- | GYTFSNY | DIHPGGGYI | SRNFAN |
| VH | (SEQ ID NO: | NYNEKFTG | (SEQ ID NO: |
| | 985) | (SEQ ID NO: | 1378) |
| | | 695) | |
| >AH04529- | GYTFSNY | DIHPGGGYI | SRNFAN |
| VH | (SEQ ID NO: | NYNEKFTG | (SEQ ID NO: |
| | 985) | (SEQ ID NO: | 1378) |
| | | 695) | |
| >AH04510- | GYTFSNY | DIHPGGGYI | SRNFAN |
| VH | (SEQ ID NO: | NYNEKFTG | (SEQ ID NO: |
| | 985) | (SEQ ID NO: | 1378) |
| | | 695) | |
| >AH04528- | GYTFSNY | DIHPGGGYI | SRNFAN |
| VH | (SEQ ID NO: | NYNEKFTG | (SEQ ID NO: |
| | 985) | (SEQ ID NO: | 1378) |
| | | 695) | |
| >AH04511- | GYTFSNY | DIHPGGGYI | SRNFAN |
| VH | (SEQ ID NO: | NYNEKFTG | (SEQ ID NO: |
| | 985) | (SEQ ID NO: | 1378) |
| | | 695) | |
| >AH04504- | GYTFTKY | DIHPGGGYI | SRNFAN |
| VH | (SEQ ID NO: | NYNEKFTG | (SEQ ID NO: |
| | 989) | (SEQ ID NO: | 1378) |
| | | 695) | |
| >AH04520- | GYTFTKY | DIHPGGGYI | SRNFAN |
| VH | (SEQ ID NO: | NYNEKFTG | (SEQ ID NO: |
| | 989) | (SEQ ID NO: | 1378) |
| | | 695) | |
| >AH04507- | GYTFTNY | DIHPGGGYI | SRNFAK |
| VH | (SEQ ID NO: | DYNEKFTG | (SEQ ID NO: |
| | 990) | (SEQ ID NO: | 1377) |
| | | 694) | |
| >AH04527- | GYTFTNY | DIHPGGGYI | SRNFAK |
| VH | (SEQ ID NO: | DYNEKFTG | (SEQ ID NO: |
| | 990) | (SEQ ID NO: | 1377) |
| | | 694) | |
| >AH04505- | GYTFTNY | DIHPGGGY | SRNFAY |
| VH | (SEQ ID NO: | TNYNEKFK | (SEQ ID NO: |
| | 990) | G (SEQ ID | 1379) |
| | | NO: 696) | |
| >AH04516- | GYTFTNY | DIHPGGGY | SRNFAY |
| VH | (SEQ ID NO: | TNYNEKFK | (SEQ ID NO: |
| | 990) | G (SEQ ID | 1379) |
| | | NO: 696) | |
| >AH04525- | GYTFTNY | DIHPGGDY | GRNFAY |
| VH | (SEQ ID NO: | TNYNEKFK | (SEQ ID NO: |
| | 990) | G (SEQ ID | 982) |
| | | NO: 693) | |
| >AH04509- | GYTFTNY | DFYPGGDY | SRNFAY |
| VH | (SEQ ID NO: | INYNEKFK | (SEQ ID NO: |
| | 990) | G (SEQ ID | 1379) |
| | | NO: 691) | |
| >AH04521- | GYTFTNY | DFYPGGDY | SRNFAY |
| VH | (SEQ ID NO: | INYNEKFK | (SEQ ID NO: |
| | 990) | G (SEQ ID | 1379) |
| | | NO: 691) | |
| >AH04526- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04514- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04524- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04530- | GYTSTNY | DIHPGGDY | SRNFAY |
| VH | (SEQ ID NO: | SNYNEKFK | (SEQ ID NO: |
| | 996) | G (SEQ ID | 1379) |
| | | NO: 692) | |
| >AH04517- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04506- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04508- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| >AH04518- | GYTFTNY | DIHPGGDY | SRNFAY |
| VH | (SEQ ID NO: | SNYNEKFK | (SEQ ID NO: |
| | 990) | G (SEQ ID | 1379) |
| | | NO: 692) | |
| AH04734 | GYTFTTST | INPRSGYT | ARHYYFDY |
| | (SEQ ID NO: | (SEQ ID NO: | (SEQ ID NO: |
| | 993) | 1024) | 612) |
| AH04750 | GYTSTAYW | ITPSTGYT | ARGGYFDY |
| | (SEQ ID NO: | (SEQ ID NO: | (SEQ ID NO: |
| | 994) | 1028) | 606) |
| AH05214 | GYTFTSYW | INPSNGRT | ARQLAAY |
| | (SEQ ID NO: | (SEQ ID NO: | (SEQ ID NO: |
| | 157) | 510) | 614) |
| AH05247 | GYTFSNYW | ILPGSGFT | ARGGTSVV |
| | (SEQ ID NO: | (SEQ ID NO: | HFDS (SEQ |
| | 986) | 1021) | ID NO: 604) |
| AH05249 | GYTFSNYW | ILPGSGFT | ARGGTSVV |
| | (SEQ ID NO: | (SEQ ID NO: | HFDS (SEQ |
| | 986) | 1021) | ID NO: 604) |
| AH05251 | GFNIKDYY | IDPDNGET | TVFWYGNN |
| | (SEQ ID NO: | (SEQ ID NO: | YAGFAY |
| | 977) | 1003) | (SEQ ID NO: |
| | | | 1420) |
| AH05256 | GYTFTRYW | INPRTDYT | ARHGYFDY |
| | (SEQ ID NO: | (SEQ ID NO: | (SEQ ID NO: |
| | 992) | 1025) | 611) |
| AH05257 | GYTFTDYA | ISTYSGDA | ARGVTFDY |
| | (SEQ ID NO: | (SEQ ID NO: | (SEQ ID NO: |
| | 19) | 21) | 23) |
| AH05258 | GYTFTDYA | ISTYSGDV | ARGVTFDS |
| | (SEQ ID NO: | (SEQ ID NO: | (SEQ ID NO: |
| | 19) | 46) | 48) |
| AH05259 | GYTFTDYA | ISTYSGDV | ARGVTFDS |
| | (SEQ ID NO: | (SEQ ID NO: | (SEQ ID NO: |
| | 19) | 46) | 48) |
| AH05268 | GYTFTDYA | ISTYSGDA | ARGVTEDY |
| | (SEQ ID NO: | (SEQ ID NO: | (SEQ ID NO: |
| | 19) | 21) | 23) |
| AH05271 | GYTFTDYA | ISTYSGDV | ARGVTFDS |
| | (SEQ ID NO: | (SEQ ID NO: | (SEQ ID NO: |
| | 19) | 46) | 48) |
| AH05274 | GYTFTNYW | IYPGGGYT | ARVTPAS |
| | (SEQ ID NO: | (SEQ ID NO: | (SEQ ID NO: |
| | 4) | 6) | 8) |
| AH05280 | GYTFSNYW | ILPGSGFT | ARGGTSVV |
| | (SEQ ID NO: | (SEQ ID NO: | HFDY (SEQ |
| | 986) | 1021) | ID NO: 605) |
| AH05285 | GYTFTRFW | INPSTDYT | ARGTVVDY |
| | (SEQ ID NO: | (SEQ ID NO: | (SEQ ID NO: |
| | 991) | 1027) | 607) |
| AH05286 | GYTFSNYW | ILPGSGYT | ARGGTSFVH |
| | (SEQ ID NO: | (SEQ ID NO: | FDY (SEQ ID |
| | 986) | 1022) | NO: 602) |
| AH4501 | GYTFTNYW | IHPGGDYS | TSRNFAY |
| | (SEQ ID NO: | (SEQ ID NO: | (SEQ ID NO: |
| | 4) | 1012) | 1419) |
| AH4502 | GYTFTNYW | IHPGGGYT | TSRNFAY |
| | (SEQ ID NO: | (SEQ ID NO: | (SEQ ID NO: |
| | 4) | 1015) | 1419) |
| AH4503 | GYTFSNYW | IHPGGGYI | VSRNFAN |
| | (SEQ ID NO: | (SEQ ID NO: | (SEQ ID NO: |
| | 986) | 1014) | 1445) |
| AH4505 | GYTFTNYW | IHPGGGYT | TSRNFAY |
| | (SEQ ID NO: | (SEQ ID NO: | (SEQ ID NO: |
| | 4) | 1015) | 1419) |
| AH4509 | GYTFTNYW | FYPGGDYI | TSRNFAY |
| | (SEQ ID NO: | (SEQ ID NO: | (SEQ ID NO: |
| | 4) | 973) | 1419) |
| AH4511 | GYTFSNYW | IHPGGGYI | VSRNFAN |
| | (SEQ ID NO: | (SEQ ID NO: | (SEQ ID NO: |
| | 986) | 1014) | 1445) |
| AH4518 | GYTFTNYW | IHPGGDYS | TSRNFAY |
| | (SEQ ID NO: | (SEQ ID NO: | (SEQ ID NO: |
| | 4) | 1012) | 1419) |
| AH4523 | GYTFTNYW | IHPGGSYT | TSRNFAK |
| | (SEQ ID NO: | (SEQ ID NO: | (SEQ ID NO: |
| | 4) | 1016) | 1418) |
| AH4524 | GYTFTNYW | IHPGGDYS | TSRNFAY |
| | (SEQ ID NO: | (SEQ ID NO: | (SEQ ID NO: |
| | 4) | 1012) | 1419) |
| AH4525 | GYTFTNYW | IHPGGDYT | TGRNFAY |
| | (SEQ ID NO: | (SEQ ID NO: | (SEQ ID NO: |
| | 4) | 1013) | 1395) |
| BP003- | GYTFTNYW | IYPGGGYT | ARVTPAS |
| T2P1C4 | (SEQ ID NO: | (SEQ ID NO: | (SEQ ID NO: |
| | 4) | 6) | 8) |
| BP003- | GYTFTNYW | IYPGGGYT | ARVTPAS |
| T2P1D10 | (SEQ ID NO: | (SEQ ID NO: | (SEQ ID NO: |
| | 4) | 6) | 8) |
| BP003- | GFNIKDYY | IDPDNGET | TVFWYGNN |
| T2P1D7 | (SEQ ID NO: | (SEQ ID NO: | YAGFAY |
| | 977) | 1003) | (SEQ ID NO: |
| | | | 1420) |
| BP003- | GYTFTNYW | IHPGGGYT | TSRNFAY |
| T2P1E3 | (SEQ ID NO: | (SEQ ID NO: | (SEQ ID NO: |
| | 4) | 1015) | 1419) |
| BP003- | GYTFTDYA | ISTYSGDA | ARGVTFDY |
| T2P1D1 | (SEQ ID NO: | (SEQ ID NO: | (SEQ ID NO: |
| | 19) | 21) | 23) |
| BL_592 | GYTFTNYW | IHPGGGYT | TSRNFAY |
| 989- | (SEQ ID NO: | (SEQ ID NO: | (SEQ ID NO: |
| 2015_G2_ | 4) | 1015) | 1419) |
| P8_G02 | | | |
| BL_592 | GYTFTSYW | IYPGSGST | TRSGVEGLL |
| 989- | (SEQ ID NO: | (SEQ ID NO: | HWYFD |
| 2016_H2_ | 157) | 251) | (SEQ ID NO: |
| P8_H02 | | | 1416) |
| BL_592 | GYTFTSYW | IYPGSGST | TRWITTDHY |
| 989- | (SEQ ID NO: | (SEQ ID NO: | FDY (SEQ ID |
| 2017_A3_ | 157) | 251) | NO: 1417) |
| P8_A03 | | | |
| BL_592 | GYTFTNYW | IYPGGGYT | ARVSPAS |
| 989- | (SEQ ID NO: | (SEQ ID NO: | (SEQ ID NO: |
| 2018_B3_ | 4) | 6) | 615) |
| P8_B03 | | | |
| BL_592 | GYTFTNYW | IYPGGGYT | ARVTPAS |
| 989- | (SEQ ID NO: | (SEQ ID NO: | (SEQ ID NO: |
| 2019_C3_ | 4) | 6) | 8) |
| P8_C03 | | | |
| BL_592 | GYTFTNYW | IHPGGGYT | TSRNFAY |
| 989- | (SEQ ID NO: | (SEQ ID NO: | (SEQ ID NO: |
| 2021_E3_ | 4) | 1015) | 1419) |
| P8_E03 | | | |
| BL_592 | GYTFTNYW | IHPGGGYT | TSRNFAY |
| 989- | (SEQ ID NO: | (SEQ ID NO: | (SEQ ID NO: |
| 2022_F3_ | 4) | 1015) | 1419) |
| P8_F03 | | | |
| BL_592 | GYTFTSYW | IYPGSGST | TRSGVEGLL |
| 989- | (SEQ ID NO: | (SEQ ID NO: | HWYFD |
| 2023_G3_ | 157) | 251) | (SEQ ID NO: |
| P8_G03 | | | 1416) |
| BL_592 | GYTSTGYW | INPSTGYT | ARGGYFDY |
| 989- | (SEQ ID NO: | (SEQ ID NO: | (SEQ ID NO: |
| 2024_H3_ | 995) | 230) | 606) |
| P8_H03 | | | |
| BL_592 | GYTFTNYW | IYPGGGYT | ARVTPAS |
| 989- | (SEQ ID NO: | (SEQ ID NO: | (SEQ ID NO: |
| 2025_A4_ | 4) | 6) | 8) |
| P8_A04 | | | |
| BL_592 | GYTFTDYA | ISTYSGDV | ARGVTFDS |
| 989- | (SEQ ID NO: | (SEQ ID NO: | (SEQ ID NO: |
| 2026_B4_ | 19) | 46) | 48) |
| P8_B04 | | | |
| BL_592 | GYTFTNYW | IYPGGGYT | ARVTPAS |
| 989- | (SEQ ID NO: | (SEQ ID NO: | (SEQ ID NO: |
| 2027_C4_ | 4) | 6) | 8) |
| P8_C04 | | | |
| BL_592 | GYTFTNYW | IHPGGDYT | TGRNFAY |
| 989- | (SEQ ID NO: | (SEQ ID NO: | (SEQ ID NO: |
| 2029_E4_ | 4) | 1013) | 1395) |
| P8_E04 | | | |
| BL_592 | GYTFTNYW | IHPGGGYT | TSRNFAY |
| 989- | (SEQ ID NO: | (SEQ ID NO: | (SEQ ID NO: |
| 2030_F4_ | 4) | 1015) | 1419) |
| P8_F04 | | | |
| BL_592 | GYTFTNYW | IHPGGGYT | TSRNFAY |
| 989- | (SEQ ID NO: | (SEQ ID NO: | (SEQ ID NO: |
| 2031_G4_ | 4) | 1015) | 1419) |
| P8_G04 | | | |
| BL_592 | GYTFSNYW | IHPGGGYI | VSRNFAN |
| 989- | (SEQ ID NO: | (SEQ ID NO: | (SEQ ID NO: |
| 2032_H4_ | 986) | 1014) | 1445) |
| P8_H04 | | | |
| BL_592 | GYTFTDYA | ISTYSGDV | ARGVTFDS |
| 989- | (SEQ ID NO: | (SEQ ID NO: | (SEQ ID NO: |
| 2033_A5_ | 19) | 46) | 48) |
| P8_A05 | | | |
| BL_592 | GYTFTNYW | IYPGGGYT | ARVTPAS |
| 989- | (SEQ ID NO: | (SEQ ID NO: | (SEQ ID NO: |
| 2034_B5_ | 4) | 6) | 8) |
| P8_B05 | | | |
| BL_592 | GYTFTNYW | IYPGGGYT | ARVTPAS |
| 989- | (SEQ ID NO: | (SEQ ID NO: | (SEQ ID NO: |
| 2035_C5_ | 4) | 6) | 8) |
| P8_C05 | | | |
| BL_592 | GYTFTNYW | IHPGGDYS | TSRNFAY |
| 989- | (SEQ ID NO: | (SEQ ID NO: | (SEQ ID NO: |
| 2037_E5_ | 4) | 1012) | 1419) |
| P8_E05 | | | |
| BL_592 | GYTFTNYW | IHPGGGYI | VSRNFAN |
| 989- | (SEQ ID NO: | (SEQ ID NO: | (SEQ ID NO: |
| 2038_F5_ | 4) | 1014) | 1445) |
| P8_F05 | | | |
| BL_592 | GYTFTNYW | IHPGGGYT | TSRNFAY |
| 989- | (SEQ ID NO: | (SEQ ID NO: | (SEQ ID NO: |
| 2039_G5_ | 4) | 1015) | 1419) |
| P8_G05 | | | |
| BL_592 | GYTFTNYW | IHPGGGYT | TSRNFAY |
| 989- | (SEQ ID NO: | (SEQ ID NO: | (SEQ ID NO: |
| 2040_H5_ | 4) | 1015) | 1419) |
| P8_H05 | | | |
| BL_592 | GYTFTDYA | ISTYSGDA | ARGVTFDY |
| 989- | (SEQ ID NO: | (SEQ ID NO: | (SEQ ID NO: |
| 2041_A6_ | 19) | 21) | 23) |
| P8_A06 | | | |
| BL_592 | GYTFTDYA | ISTYSGDV | ARGVTFDS |
| 989- | (SEQ ID NO: | (SEQ ID NO: | (SEQ ID NO: |
| 2042_B6_ | 19) | 46) | 48) |
| P8_B06 | | | |
| BL_592 | GYTFTDYA | ISTYSGDV | ARGVTFDS |
| 989- | (SEQ ID NO: | (SEQ ID NO: | (SEQ ID NO: |
| 2043_C6_ | 19) | 46) | 48) |
| P8_C06 | | | |
| BL_592 | GYTFTNYW | IYPGGGYT | ARVTPAS |
| 989- | (SEQ ID NO: | (SEQ ID NO: | (SEQ ID NO: |
| 2044_D6_ | 4) | 6) | 8) |
| P8_D06 | | | |
| BL_592 | GYTFTNYW | IHPGGDYS | TSRNFAY |
| 989- | (SEQ ID NO: | (SEQ ID NO: | (SEQ ID NO: |
| 2045_E6_ | 4) | 1012) | 1419) |
| P8_E06 | | | |
| BL_592 | GYTFTNYW | IHPGGDYS | TSRNFAY |
| 989- | (SEQ ID NO: | (SEQ ID NO: | (SEQ ID NO: |
| 2046_F6_ | 4) | 1012) | 1419) |
| P8_F06 | | | |
| BL_592 | GYTFTNYW | IHPGGGYT | TSRNFAY |
| 989- | (SEQ ID NO: | (SEQ ID NO: | (SEQ ID NO: |
| 2047_G6_ | 4) | 1015) | 1419) |
| P8_G06 | | | |
| BL_592 | GYTFTNYW | IHPGGDYS | TSRNFAY |
| 989- | (SEQ ID NO: | (SEQ ID NO: | (SEQ ID NO: |
| 2048_H6_ | 4) | 1012) | 1419) |
| P8_H06 | | | |
| BL_592 | GYTFTDYA | ISTYSGDV | ARGVTFDS |
| 989- | (SEQ ID NO: | (SEQ ID NO: | (SEQ ID NO: |
| 2049_A7_ | 19) | 46) | 48) |
| P8_A07 | | | |
| BL_592 | GYTFTNYW | IYPGGGYT | ARVTPAS |
| 989- | (SEQ ID NO: | (SEQ ID NO: | (SEQ ID NO: |
| 2051_C7_ | 4) | 6) | 8) |
| P8_C07 | | | |
| BL_592 | GFNIKDYY | IDPDNGET | TVFWYGNN |
| 989- | (SEQ ID NO: | (SEQ ID NO: | YAGFAY |
| 2052_D7_ | 977) | 1003) | (SEQ ID NO: |
| P8_D07 | | | 1420) |
| BL_592 | GYTFTNYW | IHPGGDYS | TSRNFAY |
| 989- | (SEQ ID NO: | (SEQ ID NO: | (SEQ ID NO: |
| 2054_F7_ | 4) | 1012) | 1419) |
| P8_F07 | | | |
| BL_592 | GYTFTNYW | INPGGGYT | TSRNFAY |
| 989- | (SEQ ID NO: | (SEQ ID NO: | (SEQ ID NO: |
| 2055_G7_ | 4) | 1023) | 1419) |
| P8_G07 | | | |
| BL_592 | GYTFTNYW | IYPGGGYT | VRVTPAS |
| 989- | (SEQ ID NO: | (SEQ ID NO: | (SEQ ID NO: |
| 2059_C8_ | 4) | 6) | 1443) |
| P8_C08 | | | |
| BL_592 | GYTFTDYA | ISTYSGDA | ARGVTFDY |
| 989- | (SEQ ID NO: | (SEQ ID NO: | (SEQ ID NO: |
| 2060_D8_ | 19) | 21) | 23) |
| P8_D08 | | | |
| BL_592 | GYTFTNYW | IHPGGGYI | VSRNFAN |
| 989- | (SEQ ID NO: | (SEQ ID NO: | (SEQ ID NO: |
| 2061_E8_ | 4) | 1014) | 1445) |
| P8_E08 | | | |
| BL_592 | GYTFTDYA | IITYSGDA | AXGVTFDY |
| 989- | (SEQ ID NO: | (SEQ ID NO: | (SEQ ID NO: |
| 2062_F8_ | 19) | 1020) | 619) |
| P8_F08 | | | |
| BL_592 | GYTFTSYW | IDPYDSET | AREASYYY |
| 989- | (SEQ ID NO: | (SEQ ID NO: | GNAWFA |
| 2063_G8_ | 157) | 235) | (SEQ ID NO: |
| P8_G08 | | | 601) |
| BL_592 | GYTSTNYW | IHPGGDYS | TSRNFAY |
| 989- | (SEQ ID NO: | (SEQ ID NO: | (SEQ ID NO: |
| 2064_H8_ | 997) | 1012) | 1419) |
| P8_H08 | | | |
| BL_592 | GYTFTDYA | ISTYSGDA | ARGVTFDY |
| 989- | (SEQ ID NO: | (SEQ ID NO: | (SEQ ID NO: |
| 2065_A9_ | 19) | 21) | 23) |
| P8_A09 | | | |
| BL_592 | GYAFTNY | IYPGGGYT | ARVTPAS |
| 989- | W (SEQ ID | (SEQ ID NO: | (SEQ ID NO: |
| 2067_C9_ | NO: 984) | 6) | 8) |
| P8_C09 | | | |
| BL_592 | GYTFTDYA | ISTYSGDA | ARGVTFDY |
| 989- | (SEQ ID NO: | (SEQ ID NO: | (SEQ ID NO: |
| 2068_D9_ | 19) | 21) | 23) |
| P8_D09 | | | |
| BL_592 | GYTFTNYW | IHPGGDYS | TSRNFAY |
| 989- | (SEQ ID NO: | (SEQ ID NO: | (SEQ ID NO: |
| 2069_E9_ | 4) | 1012) | 1419) |
| P8_E09 | | | |
| BL_592 | GYTFTDYA | ISTYSGDV | ARGVTFDS |
| 989- | (SEQ ID NO: | (SEQ ID NO: | (SEQ ID NO: |
| 2070_F9_ | 19) | 46) | 48) |
| P8_F09 | | | |
| BL_592 | GYTFTNYW | IHPGGGYT | TSRNFAY |
| 989- | (SEQ ID NO: | (SEQ ID NO: | (SEQ ID NO: |
| 2071_G9_ | 4) | 1015) | 1419) |
| P8_G09 | | | |
| BL_592 | GYTFTNYW | IYPGGGYT | ARVTPAS |
| 989- | (SEQ ID NO: | (SEQ ID NO: | (SEQ ID NO: |
| 2074_ | 4) | 6) | 8) |
| B10_ | | | |
| P8_B10 | | | |
| BL_592 | GYTFTNYW | IYPGGGYT | ARVTPAS |
| 989- | (SEQ ID NO: | (SEQ ID NO: | (SEQ ID NO: |
| 2076_ | 4) | 6) | 8) |
| D10_P8_ | | | |
| D10 | | | |
| BL_592 | GYTFTNYW | IHPGGGYT | TSRNFAY |
| 989- | (SEQ ID NO: | (SEQ ID NO: | (SEQ ID NO: |
| 2077_ | 4) | 1015) | 1419) |
| E10_ | | | |
| P8_E10 | | | |
| BL_592 | GYTFTNYW | IHPGGDYS | TSRNFAY |
| 989- | (SEQ ID NO: | (SEQ ID NO: | (SEQ ID NO: |
| 2078_ | 4) | 1012) | 1419) |
| F10_ | | | |
| P8_F10 | | | |
| BL_592 | GYTFTNYW | FYPGGDYI | TSRNFAY |
| 989- | (SEQ ID NO: | (SEQ ID NO: | (SEQ ID NO: |
| 2079_ | 4) | 973) | 1419) |
| G10_ | | | |
| P8_G10 | | | |
| BL_592 | GYTFTRYW | INPSTGYT | ARWGNFDY |
| 989- | (SEQ ID NO: | (SEQ ID NO: | (SEQ ID NO: |
| 2080_ | 992) | 230) | 617) |
| H10_ | | | |
| P8_H10 | | | |
| BP003- | GYTFTDYA | ISTYSGDV | ARGVTFDS |
| T2P1A7 | (SEQ ID NO: | (SEQ ID NO: | (SEQ ID NO: |
| | 19) | 46) | 48) |
| BP003- | GYTFTNYW | IYPGGGYT | VRVTPAS |
| T2P1C8 | (SEQ ID NO: | (SEQ ID NO: | (SEQ ID NO: |
| | 4) | 6) | 1443) |
| BP003- | GYTFTNYW | IHPGGGYT | TSRNFAY |
| T2P1F4 | (SEQ ID NO: | (SEQ ID NO: | (SEQ ID NO: |
| | 4) | 1015) | 1419) |
| BP003- | GYTFTNYW | IHPGGGYT | TSRNFAY |
| T2P1G2 | (SEQ ID NO: | (SEQ ID NO: | (SEQ ID NO: |
| | 4) | 1015) | 1419) |
| BP003- | GYTFTNYW | IHPGGGYT | TSRNFAY |
| T2P1G4 | (SEQ ID NO: | (SEQ ID NO: | (SEQ ID NO: |
| | 4) | 1015) | 1419) |
| BP003- | GYTFTNYW | INPGGGYT | TSRNFAY |
| T2P1G7 | (SEQ ID NO: | (SEQ ID NO: | (SEQ ID NO: |
| | 4) | 1023) | 1419) |
| BP003- | GYTFTNYW | IHPGGGYT | TSRNFAY |
| T2P1H5 | (SEQ ID NO: | (SEQ ID NO: | (SEQ ID NO: |
| | 4) | 1015) | 1419) |
| BP003T | GYTFTDYA | ISTYSGDA | ARGVTFDY |
| 3P2-1 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-10 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-11 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-12 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-13 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-14 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-15 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-16 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-17 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-18 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-19 | (SEQ ID NO: | (SEQ ID NO: | (SEQ ID NO: |
| | 19) | 21) | 23) |
| BP003T | GYTFTDYA | ISTYSGDA | ARGVTEDY |
| 3P2-2 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-20 | (SEQ ID NO: | (SEQ ID NO: | (SEQ ID NO: |
| | 19) | 21) | 23) |
| BP003T | GYTFTDYA | ISTYSGDA | ARGVTFDY |
| 3P2-21 | (SEQ ID NO: | (SEQ ID | (SEQ ID NO: |
| | 19) | NO: 21) | 23) |
| BP003T | GYTFTDYA | ISTYSGDA | ARGVTFDY |
| 3P2-22 | (SEQ ID NO: | (SEQ ID | (SEQ ID NO: |
| | 19) | NO: 21) | 23) |
| BP003T | GYTFTDYA | ISTYSGDV | ARGVTFDS |
| 3P2-23 | (SEQ ID NO: | (SEQ ID | (SEQ ID NO: |
| | 19) | NO: 46) | 48) |
| BP003T | GYTFTDYA | ISTYSGDV | ARGVTFDS |
| 3P2-24 | (SEQ ID NO: | (SEQ ID | (SEQ ID NO: |
| | 19) | NO: 46) | 48) |
| BP003T | GYTFTDYA | ISTYSGDV | ARGVTFDS |
| 3P2-25 | (SEQ ID NO: | (SEQ ID | (SEQ ID NO: |
| | 19) | NO: 46) | 48) |
| BP003T | GYTFTDYA | IXTYSGDV | ARGVTFDS |
| 3P2-26 | (SEQ ID NO: | (SEQ ID | (SEQ ID NO: |
| | 19) | NO: 1029) | 48) |
| BP003T | GYTFTDYG | ISTYSGDV | ARGVTFDS |
| 3P2-27 | (SEQ ID NO: | (SEQ ID | (SEQ ID NO: |
| | 987) | NO: 46) | 48) |
| BP003T | GYTFTRYW | INPRTDYT | ARHGYFDY |
| 3P2-28 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-29 | (SEQ ID NO: | (SEQ ID | (SEQ ID NO: |
| | 992) | NO: 1025) | 611) |
| BP003T | GYTFTDYA | ISTYSGDA | ARGVTFDY |
| 3P2-3 | (SEQ ID NO: | (SEQ ID | (SEQ ID NO: |
| | 19) | NO: 21) | 23) |
| BP003T | GYTFTRYW | INPRTDYT | ARHGYFDY |
| 3P2-30 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-31 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-32 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-33 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-34 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-35 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-36 | (SEQ ID NO: | (SEQ ID | (SEQ ID NO: |
| | 992) | NO: 1025) | 611) |
| BP003T | GYTFTRYW | INPRTDYT | ARHGYFDY |
| 3P2-37 | (SEQ ID NO: | (SEQ ID NO: | (SEQ ID NO: |
| | 992) | 1025) | 611) |
| BP003T | GYTFTRYW | INPSSDYT | ARGTVVVD |
| 3P2-38 | (SEQ ID NO: | (SEQ ID | Y (SEQ ID |
| | 992) | NO: 1026) | NO: 608) |
| BP003T | GYTFTRYW | INPSTDYT | VRSPILDY |
| 3P2-39 | (SEQ ID NO: | (SEQ ID | (SEQ ID NO: |
| | 992) | NO: 1027) | 1442) |
| BP003T | GYTFTDYA | ISTYSGDA | ARGVTFDY |
| 3P2-4 | (SEQ ID NO: | (SEQ ID | (SEQ ID NO: |
| | 19) | NO: 21) | 23) |
| BP003T | GYTFTNYW | IYPGGGYT | ARVTPAS |
| 3P2-40 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-41 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-42 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-43 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-44 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-45 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-46 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-47 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-48 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-49 | (SEQ ID NO: | (SEQ ID | (SEQ ID NO: |
| | 4) | NO: 6) | 8) |
| BP003T | GYTFTDYA | ISTYSGDA | ARGVTFDY |
| 3P2-5 | (SEQ ID NO: | (SEQ ID | (SEQ ID NO: |
| | 19) | NO: 21) | 23) |
| BP003T | GYTFTNYW | IYPGGGYT | ARVTPAS |
| 3P2-50 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-51 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-52 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-53 | (SEQ ID NO: | (SEQ ID | (SEQ ID NO: |
| | 4) | NO: 6) | 8) |
| BP003T | GYTFTNYW | IYPGGGYA | ARVTPAS |
| 3P2-54 | (SEQ ID NO: | (SEQ ID | (SEQ ID NO: |
| | 4) | NO: 1035) | 8) |
| BP003T | GYTFTNYW | IYPGGGYT | ARVTPAS |
| 3P2-55 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-56 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-57 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-58 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-59 | (SEQ ID NO: | (SEQ ID | (SEQ ID NO: |
| | 4) | NO: 6) | 8) |
| BP003T | GYTFTDYA | ISTYSGDA | ARGVTFDY |
| 3P2-6 | (SEQ ID NO: | (SEQ ID | (SEQ ID NO: |
| | 19) | NO: 21) | 23) |
| BP003T | GYTFTNYW | IYPGGGYT | ARVTPAS |
| 3P2-60 | (SEQ ID NO: | (SEQ ID | (SEQ ID NO: |
| | 4) | NO: 6) | 8) |
| BP003T | GYTFTNYW | IYPGGGYT | ARVTPAS |
| 3P2-61 | (SEQ ID NO: | (SEQ ID | (SEQ ID NO: |
| | 4) | NO: 6) | 8) |
| BP003T | GYTFSNYW | ILPGSGFT | ARGGTSVV |
| 3P2-62 | (SEQ ID NO: | (SEQ ID | HFDY (SEQ |
| | 986) | NO: 1021) | ID NO: 605) |
| BP003T | GYTFSNYW | ILPGSGFT | ARGGTSVV |
| 3P2-63 | (SEQ ID NO: | (SEQ ID | HFDS (SEQ |
| | 986) | NO: 1021) | ID NO: 604) |
| BP003T | GYTFSNYW | ILPGSGFT | ARGGTSVV |
| 3P2-64 | (SEQ ID NO: | (SEQ ID | HFDY (SEQ |
| | 986) | NO: 1021) | ID NO: 605) |
| BP003T | GYTFSNYW | ILPGSGFT | ARGGTSVV |
| 3P2-65 | (SEQ ID NO: | (SEQ ID | HFDFDY |
| | 986) | NO: 1021) | (SEQ ID NO: |
| | | | 603) |
| BP003T | GFNIKDYY | IDPENGDT | NVITTATTW |
| 3P2-66 | (SEQ ID NO: | (SEQ ID | FAY (SEQ ID |
| | 977) | NO: 1004) | NO: 1165) |
| BP003T | GFNIKDYY | IDPDNGET | TVFWYGNN |
| 3P2-67 | (SEQ ID NO: | (SEQ ID | YAGFAY |
| | 977) | NO: 1003) | (SEQ ID NO: |
| | | | 1420) |
| BP003T | GFNIKDYY | IDPDNGET | TVFWYGNN |
| 3P2-68 | (SEQ ID NO: | (SEQ ID | YAGFAY |
| | 977) | NO: 1003) | (SEQ ID NO: |
| | | | 1420) |
| BP003T | GFNIKDYY | IDPDNGET | TVFWYGNN |
| 3P2-69 | (SEQ ID NO: | (SEQ ID | YAGFAY |
| | 977) | NO: 1003) | (SEQ ID NO: |
| | | | 1420) |
| BP003T | GYTFTDYA | ISTYSGDA | ARGVTFDY |
| 3P2-7 | (SEQ ID NO: | (SEQ ID | (SEQ ID NO: |
| | 19) | NO: 21) | 23) |
| BP003T | GFNIKDYY | IDPDNGET | TVFWYGNN |
| 3P2-70 | (SEQ ID NO: | (SEQ ID | YAGFAY |
| | 977) | NO: 1003) | (SEQ ID NO: |
| | | | 1420) |
| BP003T | GFNIKDYY | IDPDNGET | TVFWYGNN |
| 3P2-71 | (SEQ ID NO: | (SEQ ID | YAGFAY |
| | 977) | NO: 1003) | (SEQ ID NO: |
| | | | 1420) |
| BP003T | GFNIKDYY | IDPDNGET | TVFWYGNN |
| 3P2-72 | (SEQ ID NO: | (SEQ ID | YAGFAY |
| | 977) | NO: 1003) | (SEQ ID NO: |
| | | | 1420) |
| BP003T | GFNIKDYY | IDPDNGET | TVFWYGNN |
| 3P2-73 | (SEQ ID NO: | (SEQ ID | YAGFAY |
| | 977) | NO: 1003) | (SEQ ID NO: |
| | | | 1420) |
| BP003T | GFNIKDYY | IDPDNGET | TVFWYGNN |
| 3P2-74 | (SEQ ID NO: | (SEQ ID | YAGFAY |
| | 977) | NO: 1003) | (SEQ ID NO: |
| | | | 1420) |
| BP003T | GYTFTDYA | ISTYSGDV | ARGVTFDS |
| 3P2-75 | (SEQ ID NO: | (SEQ ID | (SEQ ID NO: |
| | 19) | NO: 46) | 48) |
| BP003T | GYTFTDYA | ISTYSGDA | ARGVTFDY |
| 3P2-8 | (SEQ ID NO: | (SEQ ID | (SEQ ID NO: |
| | 19) | NO: 21) | 23) |
| BP003T | GYTFTDYA | ISTYSGDA | ARGVTFDY |
| 3P2-9 | (SEQ ID NO: | (SEQ ID | (SEQ ID NO: |
| | 19) | NO: 21) | 23) |
| ID | CDRL1 | CDRL2 | CDRL3 |
| D11 | QDISNY | NAK (SEQ | QHHYDTPY |
| | (SEQ ID NO: | ID NO: 28) | T (SEQ ID |
| | 27) | | NO: 30) |
| D17 | QNVGTN | SAS (SEQ | QQYNSYPLT |
| | (SEQ ID NO: | ID NO: 13) | (SEQ ID NO: |
| | 12) | | 42) |
| D34 | ENSYSY | NAK (SEQ | QHHYGTPY |
| | (SEQ ID NO: | ID NO: 28) | T (SEQ ID |
| | 52) | | NO: 54) |
| D36 | ENSYSY | NAK (SEQ | QHHYGTPY |
| | (SEQ ID NO: | ID NO: 28) | T (SEQ ID |
| | 52) | | NO: 54) |
| D5 | QNVGTN | SAS (SEQ | QQYNSYPY |
| | (SEQ ID NO: | ID NO: 13) | T (SEQ ID |
| | 12) | | NO: 15) |
| AHH03702 | QDVSTA | SAS (SEQ | QQHYSTPW |
| | (SEQ ID NO: | ID NO: 13) | T (SEQ ID |
| | 1209) | | NO: 1264) |
| AHH03703 | QDVSTA | SAS (SEQ | QQHYSTPW |
| | (SEQ ID NO: | ID NO: 13) | T (SEQ ID |
| | 1209) | | NO: 1264) |
| AHH03704 | QSLLYSSN | WAS (SEQ | QQYYSYPW |
| | QKNY (SEQ | ID NO: | T (SEQ ID |
| | ID NO: | 1450) | NO: 1294) |
| | 1304) | | |
| AHH03705 | QSLLYSSN | WAS (SEQ | QQYYSYPW |
| | QKNY (SEQ | ID NO: | T (SEQ ID |
| | ID NO: | 1450) | NO: 1294) |
| | 1304) | | |
| AHH03706 | QGISNY | YTS (SEQ | QQYSKLPW |
| | (SEQ ID NO: | ID NO: | T (SEQ ID |
| | 1211) | 1495) | NO: 1289) |
| AHH03707 | KSISKY | SGS (SEQ | QQHNEYPW |
| | (SEQ ID NO: | ID NO: | T (SEQ ID |
| | 1059) | 1365) | NO: 1257) |
| AHH03708 | QSLLDSDG | LVS (SEQ | WQGTHFPR |
| | KTY (SEQ | ID NO: | T (SEQ ID |
| | ID NO: | 1116) | NO: 1464) |
| | 1301) | | |
| AHH03709 | ENVGTY | GAS (SEQ | GQSYSYPYT |
| | (SEQ ID NO: | ID NO: | (SEQ ID NO: |
| | 933) | 975) | 981) |
| AHH03711 | QNVRTA | MAS (SEQ | LQHWNYPY |
| | (SEQ ID NO: | ID NO: | T (SEQ ID |
| | 1247) | 1122) | NO: 1093) |
| AHH03712 | QSVSND | YAS (SEQ | QQDYSSPW |
| | (SEQ ID NO: | ID NO: | T (SEQ ID |
| | 1310) | 1476) | NO: 1251) |
| AHH03713 | QEISGY | AAS (SEQ | LQYISYPRT |
| | (SEQ ID NO: | ID NO: | (SEQ ID NO: |
| | 1210) | 599) | 1107) |
| AHH03714 | QNVGTN | SAS (SEQ | QQYNSYPRT |
| | (SEQ ID NO: | ID NO: 13) | (SEQ ID NO: |
| | 12) | | 1286) |
| AHH03715 | ENIYSN | AAT (SEQ | QHFWGTPPT |
| | (SEQ ID NO: | ID NO: | (SEQ ID NO: |
| | 929) | 600) | 1212) |
| AHH03716 | QSLLDSDG | LVS (SEQ | WQGTHFRT |
| | KTY (SEQ | ID NO: | (SEQ ID NO: |
| | ID NO: | 1116) | 1466) |
| | 1301) | | |
| AHH03717 | QSLVHNNG | KVS (SEQ | SQSTHVPLT |
| | NTY (SEQ | ID NO: | (SEQ ID NO: |
| | ID NO: | 1062) | 1370) |
| | 1305) | | |
| AHH03718 | QDISNY | FTS (SEQ | QQGNTLPRT |
| | (SEQ ID NO: | ID NO: | (SEQ ID NO: |
| | 27) | 972) | 1254) |
| AHH03719 | QDIGSS | ATS (SEQ | LQYASSPYT |
| | (SEQ ID NO: | ID NO: | (SEQ ID NO: |
| | 1199) | 618) | 1101) |
| AHH03720 | QDISNY | YTS (SEQ | QQVYTLPW |
| | (SEQ ID NO: | ID NO: | T (SEQ ID |
| | 27) | 1495) | NO: 1273) |
| AHH03721 | QEISGY | AAS (SEQ | LQYASYPRT |
| | (SEQ ID NO: | ID NO: | (SEQ ID NO: |
| | 1210) | 599) | 1102) |
| AHH03722 | QEISGY | AAS (SEQ | LQYASYPRT |
| | (SEQ ID NO: | ID NO: | (SEQ ID NO: |
| | 1210) | 599) | 1102) |
| AHH03724 | QSVDYDG | AAS (SEQ | QQSNEDPYT |
| | DSY (SEQ | ID NO: | (SEQ ID NO: |
| | ID NO: | 599) | 1270) |
| | 1308) | | |
| AHH03726 | QSVDYDG | AAS (SEQ | QQSNEDPYT |
| | DSY (SEQ | ID NO: | (SEQ ID NO: |
| | ID NO: | 599) | 1270) |
| | 1308) | | |
| AHH03727 | QSLVHSNG | KVS (SEQ | SQSTHVPWT |
| | NTY (SEQ | ID NO: | (SEQ ID NO: |
| | ID NO: | 1062) | 1374) |
| | 1306) | | |
| AHH03728 | QDVNTA | SAS (SEQ | QQHFNSPYT |
| | (SEQ ID NO: | ID NO: 13) | (SEQ ID NO: |
| | 1206) | | 1256) |
| AHH03729 | QSVDYDG | ATS (SEQ | QQSNEDPLT |
| | DSY (SEQ | ID NO: | (SEQ ID NO: |
| | ID NO: | 618) | 1268) |
| | 1308) | | |
| AHH03730 | QDIGSS | ATS (SEQ | LQYASSPWT |
| | (SEQ ID NO: | ID NO: | (SEQ ID NO: |
| | 1199) | 618) | 1100) |
| AHH03731 | SSVSY (SEQ | DTS (SEQ | QQWSSNPPT |
| | ID NO: | ID NO: | (SEQ ID NO: |
| | 1385) | 839) | 1274) |
| AHH03733 | SSIGY (SEQ | DTS (SEQ | HQRGSYPW |
| | ID NO: | ID NO: | T (SEQ ID |
| | 1381) | 839) | NO: 999) |
| AHH03734 | QDVSTA | SAS (SEQ | QQHYSTPW |
| | (SEQ ID NO: | ID NO: 13) | T (SEQ ID |
| | 1209) | | NO: 1264) |
| AHH03737 | ENVATY | GAS (SEQ | GQSYRYPYT |
| | (SEQ ID NO: | ID NO: | (SEQ ID NO: |
| | 932) | 975) | 979) |
| AHH03738 | QSLVHSNG | KVS (SEQ | SQSTHVPT |
| | NTY (SEQ | ID NO: | (SEQ ID NO: |
| | ID NO: | 1062) | 1373) |
| | 1306) | | |
| AHH03739 | ESVDSYGN | LAS (SEQ | QQNNEDPY |
| | SF (SEQ ID | ID NO: | T (SEQ ID |
| | NO: 935) | 1063) | NO: 1266) |
| AHH03740 | QNVGTN | SAS (SEQ | QQYNSYPY |
| | (SEQ ID NO: | ID NO: 13) | T (SEQ ID |
| | 12) | | NO: 15) |
| AHH03742 | ENVGTY | GAS (SEQ | GQSYSYPYT |
| | (SEQ ID NO: | ID NO: | (SEQ ID NO: |
| | 933) | 975) | 981) |
| AHH03743 | QDVSTA | SAS (SEQ | QQHYSTPP |
| | (SEQ ID NO: | ID NO: 13) | WT (SEQ ID |
| | 1209) | | NO: 1262) |
| AHH03746 | QSLLYSNG | QVS (SEQ | LQGTYYPT |
| | KTY (SEQ | ID NO: | WT (SEQ ID |
| | ID NO: | 1344) | NO: 1092) |
| | 1303) | | |
| AHH03748 | QSLVHSNG | KVS (SEQ | SQSTHVPWT |
| | NTY (SEQ | ID NO: | (SEQ ID NO: |
| | ID NO: | 1062) | 1374) |
| | 1306) | | |
| AHH03749 | QDVSTA | SAS (SEQ | QQHYSTPPT |
| | (SEQ ID NO: | ID NO: 13) | (SEQ ID NO: |
| | 1209) | | 1261) |
| AHH03750 | QSLLDSDG | LVS (SEQ | WQGTHFPW |
| | KTY (SEQ | ID NO: | T (SEQ ID |
| | ID NO: | 1116) | NO: 1465) |
| | 1301) | | |
| AHH03751 | QSLLDSDG | LVS (SEQ | WQGAHFPW |
| | KTY (SEQ | ID NO: | T (SEQ ID |
| | ID NO: | 1116) | NO: 1461) |
| | 1301) | | |
| AHH03752 | QSLLDSDG | LVS (SEQ | WQGTHFPQ |
| | KTY (SEQ | ID NO: | T (SEQ ID |
| | ID NO: | 1116) | NO: 1463) |
| | 1301) | | |
| AHH03753 | QNVGSN | SAS (SEQ | QQYDSYPY |
| | (SEQ ID NO: | ID NO: 13) | T (SEQ ID |
| | 1244) | | NO: 1280) |
| AHH03754 | QDIGSS | ATS (SEQ | LQYASSPHT |
| | (SEQ ID NO: | ID NO: | (SEQ ID NO: |
| | 1199) | 618) | 1098) |
| AHH03755 | ENIYSN | AAT (SEQ | QHFWGTPW |
| | (SEQ ID NO: | ID NO: | T (SEQ ID |
| | 929) | 600) | NO: 1213) |
| AHH03756 | QDVSTA | SAS (SEQ | QQHYSTPYT |
| | (SEQ ID NO: | ID NO: 13) | (SEQ ID NO: |
| | 1209) | | 1265) |
| AHH03757 | ENIYSN | AAT (SEQ | QHFWGTPW |
| | (SEQ ID NO: | ID NO: | T (SEQ ID |
| | 929) | 600) | NO: 1213) |
| AHH03758 | TDIDDD | EGN | LQSDNMPLT |
| | (SEQ ID NO: | (SEQ ID | (SEQ ID |
| | 1391) | NO: 925) | NO: 1094) |
| AHH03759 | QSVDYDG | AAS (SEQ | QQSNEDPYT |
| | DSY (SEQ ID | ID NO: | (SEQ ID |
| | NO: 1308) | 599) | NO: 1270) |
| AHH03760 | QSVSND | YAS (SEQ | QQDYSSPPT |
| | (SEQ ID NO: | ID NO: | (SEQ ID NO: |
| | 1310) | 1476) | 1250) |
| AHH03765 | SSVSY (SEQ | DTS (SEQ | QQWSSNPP |
| | ID NO: | ID NO: | YT (SEQ ID |
| | 1385) | 839) | NO: 1275) |
| AHH03767 | QSLLDSDG | LVS (SEQ | WQGTHFPR |
| | KTY (SEQ | ID NO: | T (SEQ ID |
| | ID NO: | 1116) | NO: 1464) |
| | 1301) | | |
| AHH03768 | EDIYNR | GAT (SEQ | QQYWSTPPT |
| | (SEQ ID NO: | ID NO: | (SEQ ID NO: |
| | 924) | 976) | 1292) |
| AHH03770 | QSLLYSSN | WAS (SEQ | QQYYSYP |
| | QKNY (SEQ | ID NO: | (SEQ ID NO: |
| | ID NO: | 1450) | 1293) |
| | 1304) | | |
| AHH03771 | QSLVHSNG | KVS (SEQ | SQSTHVYT |
| | NTY (SEQ | ID NO: | (SEQ ID NO: |
| | ID NO: | 1062) | 1376) |
| | 1306) | | |
| AHH03772 | ENIYSY | NAK (SEQ | QHHYGTPW |
| | (SEQ ID NO: | ID NO: 28) | T (SEQ ID |
| | 930) | | NO: 1217) |
| AHH03773 | QSLVHSNG | KVS (SEQ | SQSTHVPT |
| | NTY (SEQ | ID NO: | (SEQ ID NO: |
| | ID NO: | 1062) | 1373) |
| | 1306) | | |
| AHH03774 | SSVSY (SEQ | DTS (SEQ | QQWSSYPY |
| | ID NO: | ID NO: | T (SEQ ID |
| | 1385) | 839) | NO: 1278) |
| AHH03776 | TDIDDD | EGN (SEQ | LQSDNMPY |
| | (SEQ ID NO: | ID NO: | T (SEQ ID |
| | 1391) | 925) | NO: 1095) |
| AHH03777 | QSLLDSDG | LVS (SEQ | WQGTFSSH |
| | KTY (SEQ | ID NO: | (SEQ ID NO: |
| | ID NO: | 1116) | 1462) |
| | 1301) | | |
| AHH03778 | QDVITA | SAS (SEQ | QQHYSTPRT |
| | (SEQ ID NO: | ID NO: 13) | (SEQ ID NO: |
| | 1205) | | 1263) |
| AHH03779 | QEISGY | AAS (SEQ | LQYASYPRT |
| | (SEQ ID NO: | ID NO: | (SEQ ID NO: |
| | 1210) | 599) | 1102) |
| AHH03780 | ESVDSYGN | RAS (SEQ | QQSNEDPRT |
| | SF (SEQ ID | ID NO: | (SEQ ID NO: |
| | NO: 935) | 1346) | 1269) |
| AHH03782 | QSLVHSNG | KVS (SEQ | SQSTHVPWT |
| | NTY (SEQ | ID NO: | (SEQ ID NO: |
| | ID NO: | 1062) | 1374) |
| | 1306) | | |
| AHH03783 | QSVDYDG | AAS (SEQ | QQSNEDPYT |
| | DSY (SEQ | ID NO: | (SEQ ID NO: |
| | ID NO: | 599) | 1270) |
| | 1308) | | |
| AHH03784 | QSLLNSSN | FAS (SEQ | QQHYSTPYT |
| | QKNY (SEQ | ID NO: | (SEQ ID NO: |
| | ID NO: | 957) | 1265) |
| | 1302) | | |
| AHH03785 | QDVSTA | SAS (SEQ | QQHYSTHV |
| | (SEQ ID NO: | ID NO: 13) | H (SEQ ID |
| | 1209) | | NO: 1259) |
| AHH03786 | QSLVHSNG | KVS (SEQ | SQSTHVPWT |
| | NTY (SEQ | ID NO: | (SEQ ID NO: |
| | ID NO: | 1062) | 1374) |
| | 1306) | | |
| AHH03787 | SSVIY (SEQ | DTS (SEQ | QQWTSNPPT |
| | ID NO: | ID NO: | (SEQ ID NO: |
| | 1383) | 839) | 1279) |
| AHH03788 | QNVGTN | SAS (SEQ | SQSTHVPYT |
| | (SEQ ID NO: | ID NO: 13) | (SEQ ID NO: |
| | 12) | | 1375) |
| AHH03790 | SRVTY | DTS (SEQ | HQRSGYSYT |
| | (SEQ ID NO: | ID NO: | (SEQ ID NO: |
| | 1380) | 839) | 1000) |
| AHH03791 | SRVTY | DTS (SEQ | HQRSGYSYT |
| | (SEQ ID NO: | ID NO: | (SEQ ID NO: |
| | 1380) | 839) | 1000) |
| AHH03792 | QNVGTN | SAS (SEQ | QQYNSYPRT |
| | (SEQ ID NO: | ID NO: 13) | (SEQ ID NO: |
| | 12) | | 1286) |
| AHH03793 | KSLLHSNG | RMS (SEQ | MQHLEYPY |
| | NTY (SEQ | ID NO: | T (SEQ ID |
| | ID NO: | 1360) | NO: 1136) |
| | 1060) | | |
| AHH03794 | QDISNY | YTS (SEQ | QQGNTLPW |
| | (SEQ ID NO: | ID NO: | T (SEQ ID |
| | 27) | 1495) | NO: 1255) |
| AHH03795 | QDISNY | YTS (SEQ | QQGNTLPW |
| | (SEQ ID NO: | ID NO: | T (SEQ ID |
| | 27) | 1495) | NO: 1255) |
| AHH03797 | QSLENSNG | RVS (SEQ | LQVTHVPFA |
| | NTY (SEQ | ID NO: | (SEQ ID NO: |
| | ID NO: | 1361) | 1096) |
| | 1300) | | |
| AHH03799 | QSLVHSYG | KVS (SEQ | SQSTHVPHT |
| | NTY (SEQ | ID NO: | (SEQ ID NO: |
| | ID NO: | 1062) | 1369) |
| | 1307) | | |
| >AH04501- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04515- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04502- | KASQNVGI | SASYRYS | QQYNSYPY |
| VH | NVA (SEQ | G (SEQ ID | T (SEQ ID |
| | ID NO: | NO: 1362) | NO: 15) |
| | 1037) | | |
| >AH04512- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04522- | KAGQNVG | SASYRYS | QQYNSYPY |
| VH | TNVA (SEQ | G (SEQ ID | T (SEQ ID |
| | ID NO: | NO: 1362) | NO: 15) |
| | 1036) | | |
| >AH04513- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04523- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04503- | KASQNVGT | SASYRYS | QQYNSYPW |
| VH | NVA (SEQ | G (SEQ ID | T (SEQ ID |
| | ID NO: | NO: 1362) | NO: 36) |
| | 1038) | | |
| >AH04529- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04510- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04528- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04511- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04504- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04520- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04507- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04527- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04505- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04516- | KASQNVGT | SASYRYS | QQYNSYPW |
| VH | NVA (SEQ | G (SEQ ID | T (SEQ ID |
| | ID NO: | NO: 1362) | NO: 36) |
| | 1038) | | |
| >AH04525- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04509- | KASQNVGT | SASYRYS | QQYNNSPLT |
| VH | NVA (SEQ | G (SEQ ID | (SEQ ID NO: |
| | ID NO: | NO: 1362) | 1282) |
| | 1038) | | |
| >AH04521- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04526- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04514- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04524- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04530- | KASQNVGT | SASYRYS | QQYNSYPW |
| VH | NVA (SEQ | G (SEQ ID | T (SEQ ID |
| | ID NO: | NO: 1362) | NO: 36) |
| | 1038) | | |
| >AH04517- | KASQSVGT | SASYRYS | QQYNNYPW |
| VH | NVA (SEQ | G (SEQ ID | T (SEQ ID |
| | ID NO: | NO: 1362) | NO: 1284) |
| | 1040) | | |
| >AH04506- | KASQNVGT | SASYRYS | QQYNSYPW |
| VH | NVA (SEQ | G (SEQ ID | T (SEQ ID |
| | ID NO: | NO: 1362) | NO: 36) |
| | 1038) | | |
| >AH04508- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| >AH04518- | KASQNVGT | SASYRYS | QQYNSYPY |
| VH | NVA (SEQ | G (SEQ ID | T (SEQ ID |
| | ID NO: | NO: 1362) | NO: 15) |
| | 1038) | | |
| AH04734 | QDIRNY | YTS (SEQ | QQGNTLPPT |
| | (SEQ ID NO: | ID NO: | (SEQ ID NO: |
| | 1202) | 1495) | 1253) |
| AH04750 | QDISNY | YTS (SEQ | QQGNTLPPT |
| | (SEQ ID NO: | ID NO: | (SEQ ID NO: |
| | 27) | 1495) | 1253) |
| AH05214 | SSISSSN | GTS (SEQ | QQWSSYPLT |
| | (SEQ ID NO: | ID NO: | (SEQ ID NO: |
| | 1382) | 983) | 1277) |
| AH05247 | QNVGTN | SAS (SEQ | QQYNSYPLT |
| | (SEQ ID NO: | ID NO: 13) | (SEQ ID NO: |
| | 12) | | 42) |
| AH05249 | QSVNND | YAS (SEQ | QQDYRSPYT |
| | (SEQ ID NO: | ID NO: | (SEQ ID NO: |
| | 1309) | 1476) | 1249) |
| AH05251 | QSIVHSNG | KVS (SEQ | FQGSHVPPT |
| | NTY (SEQ | ID NO: | (SEQ ID NO: |
| | ID NO: | 1062) | 966) |
| | 1299) | | |
| AH05256 | QDISNY | HTS (SEQ | QQSNSLPPT |
| | (SEQ ID NO: | ID NO: | (SEQ ID NO: |
| | 27) | 1002) | 1271) |
| AH05257 | QDISNY | YTS (SEQ | QQGHTLPPT |
| | (SEQ ID NO: | ID NO: | (SEQ ID NO: |
| | 27) | 1495) | 1252) |
| AH05258 | QDVSTA | SAS (SEQ | QQHYSTPFT |
| | (SEQ ID NO: | ID NO: 13) | (SEQ ID NO: |
| | 1209) | | 1260) |
| AH05259 | ENIYSF | NAK (SEQ | QHHYGIPYT |
| | (SEQ ID NO: | ID NO: 28) | (SEQ ID NO: |
| | 928) | | 1214) |
| AH05268 | QDINRY | RAN (SEQ | LQYDEFPYT |
| | (SEQ ID NO: | ID NO: | (SEQ ID NO: |
| | 1201) | 1345) | 1106) |
| AH05271 | ENIYFS | NAN (SEQ | KQAYDVPW |
| | (SEQ ID NO: | ID NO: | T (SEQ ID |
| | 927) | 1142) | NO: 1058) |
| AH05274 | QNVGTN | SAS (SEQ | QQYNSYPY |
| | (SEQ ID NO: | ID NO: 13) | T (SEQ ID |
| | 12) | | NO: 15) |
| AH05280 | QSVNND | YAS (SEQ | QQAYWSPY |
| | (SEQ ID NO: | ID NO: | T (SEQ ID |
| | 1309) | 1476) | NO: 1248) |
| AH05285 | QDISNY | YTS (SEQ | QQGNTLPPT |
| | (SEQ ID NO: | ID NO: | (SEQ ID NO: |
| | 27) | 1495) | 1253) |
| AH05286 | QDVNTA | WAS (SEQ | QQHYSSPW |
| | (SEQ ID NO: | ID NO: | T (SEQ ID |
| | 1206) | 1450) | NO: 1258) |
| AH4501 | QNVGTN | SAS (SEQ | QQYNSYPY |
| | (SEQ ID NO: | ID NO: 13) | T (SEQ ID |
| | 12) | | NO: 15) |
| AH4502 | QNVGIN | SAS (SEQ | QQYNSYPY |
| | (SEQ ID NO: | ID NO: 13) | T (SEQ ID |
| | 1243) | | NO: 15) |
| AH4503 | QNVGTN | SAS (SEQ | QQYNSYPW |
| | (SEQ ID NO: | ID NO: 13) | T (SEQ ID |
| | 12) | | NO: 36) |
| AH4505 | QNVGTN | SAS (SEQ | QQYNSYPY |
| | (SEQ ID NO: | ID NO: 13) | T (SEQ ID |
| | 12) | | NO: 15) |
| AH4509 | QNVGTN | SAS (SEQ | QQYNNSPLT |
| | (SEQ ID NO: | ID NO: 13) | (SEQ ID NO: |
| | 12) | | 1282) |
| AH4511 | QNVGTN | SAS (SEQ | QQYNSYPY |
| | (SEQ ID NO: | ID NO: 13) | T (SEQ ID |
| | 12) | | NO: 15) |
| AH4518 | QNVGTN | SAS (SEQ | QQYNSYPY |
| | (SEQ ID NO: | ID NO: 13) | T (SEQ ID |
| | 12) | | NO: 15) |
| AH4523 | QNVGTN | SAS (SEQ | QQYNSYPY |
| | (SEQ ID NO: | ID NO: 13) | T (SEQ ID |
| | 12) | | NO: 15) |
| AH4524 | QNVGTN | SAS (SEQ | QQYNSYPY |
| | (SEQ ID NO: | ID NO: 13) | T (SEQ ID |
| | 12) | | NO: 15) |
| AH4525 | QNVGTN | SAS (SEQ | QQYNSYPY |
| | (SEQ ID NO: | ID NO: 13) | T (SEQ ID |
| | 12) | | NO: 15) |
| BP003- | QNVGTN | SAS (SEQ | QQYNSYPY |
| T2P1C4 | (SEQ ID NO: | ID NO: 13) | T (SEQ ID |
| | 12) | | NO: 15) |
| BP003- | QNVGTN | SAS (SEQ | QQYNRYPY |
| T2P1D10 | (SEQ ID NO: | ID NO: 13) | T (SEQ ID |
| | 12) | | NO: 1285) |
| BP003- | QSIVHSNG | KVS (SEQ | FQGSHVPPT |
| T2P1D7 | NTY (SEQ | ID NO: | (SEQ ID NO: |
| | ID NO: | 1062) | 966) |
| | 1299) | | |
| BP003- | QNVGTN | SAS (SEQ | QQYNSYPY |
| T2P1E3 | (SEQ ID NO: | ID NO: 13) | T (SEQ ID |
| | 12) | | NO: 15) |
| BP003- | ENSYSY | NAK (SEQ | QHHYGTPY |
| T2P1D1 | (SEQ ID NO: | ID NO: 28) | T (SEQ ID |
| | 52) | | NO: 54) |
| BL_592 | QNVGTN | SAS (SEQ | QQYNSYPY |
| 989- | (SEQ ID NO: | ID NO: 13) | T (SEQ ID |
| 2015_G2_ | 12) | | NO: 15) |
| P8_G02 | | | |
| BL_592 | QSLVHSNG | EVS (SEQ | SQSTHVPYT |
| 989- | NTY (SEQ | ID NO: | (SEQ ID NO: |
| 2016_H2_ | ID NO: | 944) | 1375) |
| P8_H02 | 1306) | | |
| BL_592 | QDVNTAV | WAS (SEQ | QQHYSSPW |
| 989- | A (SEQ ID | ID NO: | T (SEQ ID |
| 2017_A3_ | NO: 1207) | 1450) | NO: 1258) |
| P8_A03 | | | |
| BL_592 | QNVGTN | SAS (SEQ | QQYNSYPY |
| 989- | (SEQ ID NO: | ID NO: 13) | T (SEQ ID |
| 2018_B3_ | 12) | | NO: 15) |
| P8_B03 | | | |
| BL_592 | QNVGTN | SAS (SEQ | QQYNSYPY |
| 989- | (SEQ ID NO: | ID NO: 13) | T (SEQ ID |
| 2019_C3_ | 12) | | NO: 15) |
| P8_C03 | | | |
| BL_592 | QNVGTN | SAS (SEQ | QQYNSYPY |
| 989- | (SEQ ID NO: | ID NO: 13) | T (SEQ ID |
| 2021_E3_ | 12) | | NO: 15) |
| P8_E03 | | | |
| BL_592 | QNVGTN | SAS (SEQ | QQYNSYPY |
| 989- | (SEQ ID NO: | ID NO: 13) | T (SEQ ID |
| 2022_F3_ | 12) | | NO: 15) |
| P8_F03 | | | |
| BL_592 | QSLVHSNG | EVS (SEQ | SQSTHVPYT |
| 989- | NTY (SEQ | ID NO: | (SEQ ID NO: |
| 2023_G3_ | ID NO: | 944) | 1375) |
| P8_G03 | 1306) | | |
| BL_592 | QDIRNY | YTS (SEQ | QQGNTLPPT |
| 989- | (SEQ ID NO: | ID NO: | (SEQ ID NO: |
| 2024_H3_ | 1202) | 1495) | 1253) |
| P8_H03 | | | |
| BL_592 | QNVGTN | SAS (SEQ | QQYNSYPY |
| 989- | (SEQ ID NO: | ID NO: 13) | T (SEQ ID |
| 2025_A4_ | 12) | | NO: 15) |
| P8_A04 | | | |
| BL_592 | ENIYSY | NAK (SEQ | QHHYGSPY |
| 989- | (SEQ ID NO: | ID NO: 28) | T (SEQ ID |
| 2026_B4_ | 930) | | NO: 1216) |
| P8_B04 | | | |
| BL_592 | QNVGTN | SAS (SEQ | QQYNSYPY |
| 989- | (SEQ ID NO: | ID NO: 13) | T (SEQ ID |
| 2027_C4_ | 12) | | NO: 15) |
| P8_C04 | | | |
| BL_592 | QNVGTN | SAS (SEQ | QQYNSYPY |
| 989- | (SEQ ID NO: | ID NO: 13) | T (SEQ ID |
| 2029_E4_ | 12) | | NO: 15) |
| P8_E04 | | | |
| BL_592 | QNVGTN | SAS (SEQ | QQYNSYPW |
| 989- | (SEQ ID NO: | ID NO: 13) | T (SEQ ID |
| 2030_F4_ | 12) | | NO: 36) |
| P8_F04 | | | |
| BL_592 | QNVGTN | SAS (SEQ | QQYNSYPY |
| 989- | (SEQ ID NO: | ID NO: 13) | T (SEQ ID |
| 2031_G4_ | 12) | | NO: 15) |
| P8_G04 | | | |
| BL_592 | QNVGTN | SAS (SEQ | QQYNSYPY |
| 989- | (SEQ ID NO: | ID NO: 13) | T (SEQ ID |
| 2032_H4_ | 12) | | NO: 15) |
| P8_H04 | | | |
| BL_592 | ENIYSY | NAK (SEQ | QHHYGIPYT |
| 989- | (SEQ ID NO: | ID NO: 28) | (SEQ ID NO: |
| 2033_A5_ | 930) | | 1214) |
| P8_A05 | | | |
| BL_592 | QNVGTN | SAS (SEQ | QQYNSYPY |
| 989- | (SEQ ID NO: | ID NO: 13) | T (SEQ ID |
| 2034_B5_ | 12) | | NO: 15) |
| P8_B05 | | | |
| BL_592 | QNVGTN | SAS (SEQ | QQYNSYPY |
| 989- | (SEQ ID NO: | ID NO: 13) | T (SEQ ID |
| 2035_C5_ | 12) | | NO: 15) |
| P8_C05 | | | |
| BL_592 | QNVGTN | SAS (SEQ | QQYNSYPY |
| 989- | (SEQ ID NO: | ID NO: 13) | T (SEQ ID |
| 2037_E5_ | 12) | | NO: 15) |
| P8_E05 | | | |
| BL_592 | QNVGTN | SAS (SEQ | QQYNSYPY |
| 989- | (SEQ ID NO: | ID NO: 13) | T (SEQ ID |
| 2038_F5_ | 12) | | NO: 15) |
| P8_F05 | | | |
| BL_592 | QNVGTN | SAS (SEQ | QQYNNYPW |
| 989- | (SEQ ID NO: | ID NO: 13) | T (SEQ ID |
| 2039_G5_ | 12) | | NO: 1284) |
| P8_G05 | | | |
| BL_592 | QNVGTN | SAS (SEQ | QQYNSYPY |
| 989- | (SEQ ID NO: | ID NO: 13) | T (SEQ ID |
| 2040_H5_ | 12) | | NO: 15) |
| P8_H05 | | | |
| BL_592 | ENSYSY | NAK (SEQ | QHHYGTPY |
| 989- | (SEQ ID NO: | ID NO: 28) | T (SEQ ID |
| 2041_A6_ | 52) | | NO: 54) |
| P8_A06 | | | |
| BL_592 | ENSYSY | NAK (SEQ | QHHYGTPY |
| 989- | (SEQ ID NO: | ID NO: 28) | T (SEQ ID |
| 2042_B6_ | 52) | | NO: 54) |
| P8_B06 | | | |
| BL_592 | ENIYSY | NAK (SEQ | QHHYGTPY |
| 989- | (SEQ ID NO: | ID NO: 28) | T (SEQ ID |
| 2043_C6_ | 930) | | NO: 54) |
| P8_C06 | | | |
| BL_592 | QNVGTN | SAS (SEQ | QQYNTYPY |
| 989- | (SEQ ID NO: | ID NO: 13) | T (SEQ ID |
| 2044_D6_ | 12) | | NO: 1287) |
| P8_D06 | | | |
| BL_592 | QNVGTN | SAS (SEQ | QQYNSYPY |
| 989- | (SEQ ID NO: | ID NO: 13) | T (SEQ ID |
| 2045_E6_ | 12) | | NO: 15) |
| P8_E06 | | | |
| BL_592 | QNVGTN | SAS (SEQ | QQYNSYPY |
| 989- | (SEQ ID NO: | ID NO: 13) | T (SEQ ID |
| 2046_F6_ | 12) | | NO: 15) |
| P8_F06 | | | |
| BL_592 | QNVGTN | SAS (SEQ | QQYNNYPW |
| 989- | (SEQ ID NO: | ID NO: 13) | T (SEQ ID |
| 2047_G6_ | 12) | | NO: 1284) |
| P8_G06 | | | |
| BL_592 | QNVGTN | SAS (SEQ | QQYNSYPY |
| 989- | (SEQ ID NO: | ID NO: 13) | T (SEQ ID |
| 2048_H6_ | 12) | | NO: 15) |
| P8_H06 | | | |
| BL_592 | ENIYSY | NAK (SEQ | QHHYGTPY |
| 989- | (SEQ ID NO: | ID NO: 28) | T (SEQ ID |
| 2049_A7_ | 930) | | NO: 54) |
| P8_A07 | | | |
| BL_592 | QNVGTN | SAS (SEQ | QQYNSYPY |
| 989- | (SEQ ID NO: | ID NO: 13) | T (SEQ ID |
| 2051_C7_ | 12) | | NO: 15) |
| P8_C07 | | | |
| BL_592 | QSIVHSNG | KVS (SEQ | FQGSHVPPT |
| 989- | NTY (SEQ | ID NO: | (SEQ ID NO: |
| 2052_D7_ | ID NO: | 1062) | 966) |
| P8_D07 | 1299) | | |
| BL_592 | QNVGTN | SAS (SEQ | QQYNNHPY |
| 989- | (SEQ ID NO: | ID NO: 13) | T (SEQ ID |
| 2054_F7_ | 12) | | NO: 1281) |
| P8_F07 | | | |
| BL_592 | QNVGTN | SAS (SEQ | QQYNSYPW |
| 989- | (SEQ ID NO: | ID NO: 13) | T (SEQ ID |
| 2055_G7_ | 12) | | NO: 36) |
| P8_G07 | | | |
| BL_592 | QNVGTN | SAS (SEQ | QQYNTYPY |
| 989- | (SEQ ID NO: | ID NO: 13) | T (SEQ ID |
| 2059_C8_ | 12) | | NO: 1287) |
| P8_C08 | | | |
| BL_592 | ENIYSY | NAK (SEQ | QHHYGSPY |
| 989- | (SEQ ID NO: | ID NO: 28) | T (SEQ ID |
| 2060_D8_ | 930) | | NO: 1216) |
| P8_D08 | | | |
| BL_592 | QNVGTN | SAS (SEQ | QQYNSYPY |
| 989- | (SEQ ID NO: | ID NO: 13) | T (SEQ ID |
| 2061_E8_ | 12) | | NO: 15) |
| P8_E08 | | | |
| BL_592 | ENIYSF | NAK (SEQ | QHHYGTPY |
| 989- | (SEQ ID NO: | ID NO: 28) | T (SEQ ID |
| 2062_F8_ | 928) | | NO: 54) |
| P8_F08 | | | |
| BL_592 | QNVGTNV | SAS (SEQ | QQYNSYPY |
| 989- | A (SEQ ID | ID NO: 13) | T (SEQ ID |
| 2063_G8_ | NO: 1246) | | NO: 15) |
| P8_G08 | | | |
| BL_592 | QNVGTN | SAS (SEQ | QQYNSYPW |
| 989- | (SEQ ID NO: | ID NO: 13) | T (SEQ ID |
| 2064_H8_ | 12) | | NO: 36) |
| P8_H08 | | | |
| BL_592 | ENIYSF | NAK (SEQ | QHHYGTPY |
| 989- | (SEQ ID NO: | ID NO: 28) | T (SEQ ID |
| 2065_A9_ | 928) | | NO: 54) |
| P8_A09 | | | |
| BL_592 | QNVGTN | SAS (SEQ | QQYNNYPL |
| 989- | (SEQ ID NO: | ID NO: 13) | T (SEQ ID |
| 2067_C9_ | 12) | | NO: 1283) |
| P8_C09 | | | |
| BL_592 | ENIYSY | NAK (SEQ | QHHYDTPY |
| 989- | (SEQ ID NO: | ID NO: 28) | T (SEQ ID |
| 2068_D9_ | 930) | | NO: 30) |
| P8_D09 | | | |
| BL_592 | QNVGTN | SAS (SEQ | QQYNSYPY |
| 989- | (SEQ ID NO: | ID NO: 13) | T (SEQ ID |
| 2069_E9_ | 12) | | NO: 15) |
| P8_E09 | | | |
| BL_592 | ENIYSY | NAK (SEQ | QHHYGSPY |
| 989- | (SEQ ID NO: | ID NO: 28) | T (SEQ ID |
| 2070_F9_ | 930) | | NO: 1216) |
| P8_F09 | | | |
| BL_592 | QNVGTN | SAS (SEQ | QQYNSYPY |
| 989- | (SEQ ID NO: | ID NO: 13) | T (SEQ ID |
| 2071_G9_ | 12) | | NO: 15) |
| P8_G09 | | | |
| BL_592 | QNVGTN | SAS (SEQ | QQYNSYPY |
| 989- | (SEQ ID NO: | ID NO: 13) | T (SEQ ID |
| 2074__ | 12) | | NO: 15) |
| B10_ | | | |
| P8_B10 | | | |
| BL_592 | QNVGTN | SAS (SEQ | QQYNRYPY |
| 989- | (SEQ ID NO: | ID NO: 13) | T (SEQ ID |
| 2076_ | 12) | | NO: 1285) |
| D10_P8_ | | | |
| D10 | | | |
| BL_592 | QNVGTN | SAS (SEQ | QQYNSYPW |
| 989- | (SEQ ID NO: | ID NO: 13) | T (SEQ ID |
| 2077_ | 12) | | NO: 36) |
| E10_ | | | |
| P8_E10 | | | |
| BL_592 | QNVGTN | SAS (SEQ | QQYNSYPY |
| 989- | (SEQ ID NO: | ID NO: 13) | T (SEQ ID |
| 2078_ | 12) | | NO: 15) |
| F10_ | | | |
| P8_F10 | | | |
| BL_592 | QNVGTN | SAS (SEQ | QQYNSYPW |
| 989- | (SEQ ID NO: | ID NO: 13) | T (SEQ ID |
| 2079_ | 12) | | NO: 36) |
| G10_ | | | |
| P8_G10 | | | |
| BL_592 | QDIRNY | YTS (SEQ | QQGNTLPPT |
| 989- | (SEQ ID NO: | ID NO: | (SEQ ID NO: |
| 2080_ | 1202) | 1495) | 1253) |
| H10_ | | | |
| P8_H10 | | | |
| BP003- | ENIYSY | NAK (SEQ | QHHYGTPY |
| T2P1A7 | (SEQ ID NO: | ID NO: 28) | T (SEQ ID |
| | 930) | | NO: 54) |
| BP003- | QNVGTN | SAS (SEQ | QQYNTYPY |
| T2P1C8 | SEQ ID NO: | ID NO: 13) | T (SEQ ID |
| | 12) | | NO: 1287) |
| BP003- | QNVGTN | SAS (SEQ | QQYNSYPW |
| T2P1F4 | (SEQ ID NO: | ID NO: 13) | T (SEQ ID |
| | 12) | | NO: 36) |
| BP003- | QNVGTN | SAS (SEQ | QQYNSYPY |
| T2P1G2 | (SEQ ID NO: | ID NO: 13) | T (SEQ ID |
| | 12) | | NO: 15) |
| BP003- | QNVGTN | SAS (SEQ | QQYNSYPY |
| T2P1G4 | SEQ ID NO: | ID NO: 13) | T (SEQ ID |
| | 12) | | NO: 15) |
| BP003- | QNVGTN | SAS (SEQ | QQYNSYPW |
| T2P1G7 | (SEQ ID NO: | ID NO: 13) | T (SEQ ID |
| | 12) | | NO: 36) |
| BP003- | QNVGTN | SAS (SEQ | QQYNSYPY |
| T2P1H5 | (SEQ ID NO: | ID NO: 13) | T (SEQ ID |
| | 12) | | NO: 15) |
| BP003T | ENIYSY | NAK (SEQ | QHHYDTPY |
| 3P2-1 | (SEQ ID NO: | ID NO: 28) | T (SEQ ID |
| | 930) | | NO: 30) |
| BP003T | QDINRY | RAN (SEQ | LQYDEFPYT |
| 3P2-10 | (SEQ ID NO: | ID NO: | (SEQ ID NO: |
| | 1201) | 1345) | 1106) |
| BP003T | QDINRY | RAN (SEQ | LQYDEFPYT |
| 3P2-11 | (SEQ ID NO: | ID NO: | (SEQ ID NO: |
| | 1201) | 1345) | 1106) |
| BP003T | QDINNF | RAN (SEQ | LQYDEFPW |
| 3P2-12 | (SEQ ID NO: | ID NO: | T (SEQ ID |
| | 1200) | 1345) | NO: 1105) |
| BP003T | ENIYSY | NAK (SEQ | QHHYGSPY |
| 3P2-13 | (SEQ ID NO: | ID NO: 28) | T (SEQ ID |
| | 930) | | NO: 1216) |
| BP003T | ENIYSY | NAK (SEQ | QHHYDTPY |
| 3P2-14 | (SEQ ID NO: | ID NO: 28) | T (SEQ ID |
| | 930) | | NO: 30) |
| BP003T | ENSYSY | NAK (SEQ | QHHYGTPY |
| 3P2-15 | (SEQ ID NO: | ID NO: 28) | T (SEQ ID |
| | 52) | | NO: 54) |
| BP003T | ENIYSY | NAK (SEQ | QHHYDTPY |
| 3P2-16 | (SEQ ID NO: | ID NO: 28) | T (SEQ ID |
| | 930) | | NO: 30) |
| BP003T | ENIYSY | NAK (SEQ | QHHYDTPY |
| 3P2-17 | (SEQ ID NO: | ID NO: 28) | T (SEQ ID |
| | 930) | | NO: 30) |
| BP003T | ENIYSY | NAK (SEQ | QHHYGSPY |
| 3P2-18 | (SEQ ID NO: | ID NO: 28) | T (SEQ ID |
| | 930) | | NO: 1216) |
| BP003T | ENIYSY | NAK (SEQ | QHHYDTPY |
| 3P2-19 | (SEQ ID NO: | ID NO: 28) | T (SEQ ID |
| | 930) | | NO: 30) |
| BP003T | ENIYSY | NAK (SEQ | QHHYDTPY |
| 3P2-2 | (SEQ ID NO: | ID NO: 28) | T (SEQ ID |
| | 930) | | NO: 30) |
| BP003T | KNSYSY | NAK (SEQ | QHHYGTPY |
| 3P2-20 | (SEQ ID NO: | ID NO: 28) | T (SEQ ID |
| | 1057) | | NO: 54) |
| BP003T | ENIYSY | NAK (SEQ | QHHYGTPY |
| 3P2-21 | (SEQ ID NO: | ID NO: 28) | T (SEQ ID |
| | 930) | | NO: 54) |
| BP003T | ENSYSY | NAK (SEQ | QHHYGTPY |
| 3P2-22 | (SEQ ID NO: | ID NO: 28) | T (SEQ ID |
| | 52) | | NO: 54) |
| BP003T | ENSYSY | NAK (SEQ | QHHYGSPY |
| 3P2-23 | (SEQ ID NO: | ID NO: 28) | T (SEQ ID |
| | 52) | | NO: 1216) |
| BP003T | ENSYSY | NAK (SEQ | QHHYGSPY |
| 3P2-24 | (SEQ ID NO: | ID NO: 28) | T (SEQ ID |
| | 52) | | NO: 1216) |
| BP003T | ENSYSY | NAK (SEQ | QHHYGSPY |
| 3P2-25 | (SEQ ID NO: | ID NO: 28) | T (SEQ ID |
| | 52) | | NO: 1216) |
| BP003T | ENIYSY | NAK (SEQ | QHHYGSPY |
| 3P2-26 | (SEQ ID NO: | ID NO: 28) | T (SEQ ID |
| | 930) | | NO: 1216) |
| BP003T | QDISNY | YAS (SEQ | QQGNTLPW |
| 3P2-27 | (SEQ ID NO: | ID NO: | T (SEQ ID |
| | 27) | 1476) | NO: 1255) |
| BP003T | QDISNY | HTS (SEQ | QQSNTLPPT |
| 3P2-28 | (SEQ ID NO: | ID NO: | (SEQ ID NO: |
| | 27) | 1002) | 1272) |
| BP003T | QDISNY | HTS (SEQ | QQSNSLPPT |
| 3P2-29 | (SEQ ID NO: | ID NO: | (SEQ ID NO: |
| | 27) | 1002) | 1271) |
| BP003T | ENIYSY | NAK (SEQ | QHHYDTPY |
| 3P2-3 | (SEQ ID NO: | ID NO: 28) | T (SEQ ID |
| | 930) | | NO: 30) |
| BP003T | QDISNY | HTS (SEQ | QQSNSLPPT |
| 3P2-30 | (SEQ ID NO: | ID NO: | (SEQ ID NO: |
| | 27) | 1002) | 1271) |
| BP003T | QDISNY | HTS (SEQ | QQSNSLPPT |
| 3P2-31 | (SEQ ID NO: | ID NO: | (SEQ ID NO: |
| | 27) | 1002) | 1271) |
| BP003T | QDISNY | YTS (SEQ | QQGNTLPPT |
| 3P2-32 | (SEQ ID NO: | ID NO: | (SEQ ID NO: |
| | 27) | 1495) | 1253) |
| BP003T | QDISNY | HTS (SEQ | QQSNSLPPT |
| 3P2-33 | (SEQ ID NO: | ID NO: | (SEQ ID NO: |
| | 27) | 1002) | 1271) |
| BP003T | QDISNY | HTS (SEQ | QQSNSLPPT |
| 3P2-34 | (SEQ ID NO: | ID NO: | (SEQ ID NO: |
| | 27) | 1002) | 1271) |
| BP003T | QDISNY | HTS (SEQ | QQSNTLPPT |
| 3P2-35 | (SEQ ID NO: | ID NO: | SEQ ID NO: |
| | 27) | 1002) | 1272) |
| BP003T | QDISNY | HTS (SEQ | QQSNTLPPT |
| 3P2-36 | (SEQ ID NO: | ID NO: | (SEQ ID NO: |
| | 27) | 1002) | 1272) |
| BP003T | QDISNY | HTS (SEQ | QQGNTLPPT |
| 3P2-37 | (SEQ ID NO: | ID NO: | (SEQ ID NO: |
| | 27) | 1002) | 1253) |
| BP003T | QDISNY | YTS (SEQ | QQGNTLPPT |
| 3P2-38 | (SEQ ID NO: | ID NO: | (SEQ ID NO: |
| | 27) | 1495) | 1253) |
| BP003T | QDISNY | HTS (SEQ | QQGNTLPPT |
| 3P2-39 | (SEQ ID NO: | ID NO: | (SEQ ID NO: |
| | 27) | 1002) | 1253) |
| BP003T | ENIYSY | NAK (SEQ | QHHYDTPY |
| 3P2-4 | (SEQ ID NO: | ID NO: 28) | T (SEQ ID |
| | 930) | | NO: 30) |
| BP003T | QNVGTN | SAS (SEQ | QQYNSYPY |
| 3P2-40 | (SEQ ID NO: | ID NO: 13) | T (SEQ ID |
| | 12) | | NO: 15) |
| BP003T | QNVGTN | SAS (SEQ | QQYNSYPY |
| 3P2-41 | (SEQ ID NO: | ID NO: 13) | T (SEQ ID |
| | 12) | | NO: 15) |
| BP003T | QNVGTN | SAS (SEQ | QQYNSYPY |
| 3P2-42 | (SEQ ID NO: | ID NO: 13) | T (SEQ ID |
| | 12) | | NO: 15) |
| BP003T | QNVGTN | SAS (SEQ | QQYNTYPY |
| 3P2-43 | (SEQ ID NO: | ID NO: 13) | T (SEQ ID |
| | 12) | | NO: 1287) |
| BP003T | QNVGTN | SAS (SEQ | QQYNSYPY |
| 3P2-44 | (SEQ ID NO: | ID NO: 13) | T (SEQ ID |
| | 12) | | NO: 15) |
| BP003T | QNVGTN | SAS (SEQ | QQYNTYPY |
| 3P2-45 | (SEQ ID NO: | ID NO: 13) | T (SEQ ID |
| | 12) | | NO: 1287) |
| BP003T | QDIGLN | ATS (SEQ | LQYASSPFT |
| 3P2-46 | (SEQ ID NO: | ID NO: | (SEQ ID NO: |
| | 1198) | 618) | 1097) |
| BP003T | QNVGTN | SAS (SEQ | QQYNTYPY |
| 3P2-47 | (SEQ ID NO: | ID NO: 13) | T (SEQ ID |
| | 12) | | NO: 1287) |
| BP003T | QNVGTN | SAS (SEQ | QQYNSYPY |
| 3P2-48 | (SEQ ID NO: | ID NO: 13) | T (SEQ ID |
| | 12) | | NO: 15) |
| BP003T | QNVGTN | SAS (SEQ | QQYNSYPY |
| 3P2-49 | (SEQ ID NO: | ID NO: 13) | T (SEQ ID |
| | 12) | | NO: 15) |
| BP003T | ENIYSY | NAK (SEQ | QHHYDTPY |
| 3P2-5 | (SEQ ID NO: | ID NO: 28) | T (SEQ ID |
| | 930) | | NO: 30) |
| BP003T | QNVGTN | SAS (SEQ | QQYNSYPY |
| 3P2-50 | (SEQ ID NO: | ID NO: 13) | T (SEQ ID |
| | 12) | | NO: 15) |
| BP003T | QSLVHSNG | KVS (SEQ | SQSTHVPPT |
| 3P2-51 | NTY (SEQ | ID NO: | (SEQ ID NO: |
| | ID NO: | 1062) | 1371) |
| | 1306) | | |
| BP003T | QNVGTN | SAS (SEQ | QQYNSYPY |
| 3P2-52 | (SEQ ID NO: | ID NO: 13) | T (SEQ ID |
| | 12) | | NO: 15) |
| BP003T | QNVGTN | SAS (SEQ | QQYNSYPY |
| 3P2-53 | (SEQ ID NO: | ID NO: 13) | T (SEQ ID |
| | 12) | | NO: 15) |
| BP003T | QNVGTN | SAS (SEQ | QQYNSYPY |
| 3P2-54 | (SEQ ID NO: | ID NO: 13) | T (SEQ ID |
| | 12) | | NO: 15) |
| BP003T | QNVGTN | SAS (SEQ | QQYNSYPY |
| 3P2-55 | (SEQ ID NO: | ID NO: 13) | T (SEQ ID |
| | 12) | | NO: 15) |
| BP003T | QNVGTN | SAS (SEQ | QQYNSYPY |
| 3P2-56 | (SEQ ID NO: | ID NO: 13) | T (SEQ ID |
| | 12) | | NO: 15) |
| BP003T | QNVGTN | SAS (SEQ | QQYNSYPY |
| 3P2-57 | (SEQ ID NO: | ID NO: 13) | T (SEQ ID |
| | 12) | | NO: 15) |
| BP003T | QNVGTN | SAS (SEQ | QQYNSYPY |
| 3P2-58 | (SEQ ID NO: | ID NO: 13) | T (SEQ ID |
| | 12) | | NO: 15) |
| BP003T | QDVRTA | STS (SEQ | QQYSNYLTF |
| 3P2-59 | (SEQ ID NO: | ID NO: | (SEQ ID NO: |
| | 1208) | 1386) | 1290) |
| BP003T | ENIYSY | NAK (SEQ | QHHYDTPY |
| 3P2-6 | (SEQ ID NO: | ID NO: 28) | T (SEQ ID |
| | 930) | | NO: 30) |
| BP003T | QNVGTN | SAS (SEQ | QQYNTYPY |
| 3P2-60 | (SEQ ID NO: | ID NO: 13) | T (SEQ ID |
| | 12) | | NO: 1287) |
| BP003T | QNVGTN | SAS (SEQ | XQYNSYPY |
| 3P2-61 | (SEQ ID NO: | ID NO: 13) | T (SEQ ID |
| | 12) | | NO: 1475) |
| BP003T | QSVNND | YAS (SEQ | QQAYWSPY |
| 3P2-62 | (SEQ ID NO: | ID NO: | T (SEQ ID |
| | 1309) | 1476) | NO: 1248) |
| BP003T | QDVNTA | WAS (SEQ | QQHYSSPW |
| 3P2-63 | (SEQ ID NO: | ID NO: | T (SEQ ID |
| | 1206) | 1450) | NO: 1258) |
| BP003T | QDVNTA | WAS (SEQ | QQHYSSPW |
| 3P2-64 | (SEQ ID NO: | ID NO: | T (SEQ ID |
| | 1206) | 1450) | NO: 1258) |
| BP003T | QDVNTA | WAS (SEQ | QQHYSSPW |
| 3P2-65 | (SEQ ID NO: | ID NO: | T (SEQ ID |
| | 1206) | 1450) | NO: 1258) |
| BP003T | QEISGY | AAS (SEQ | LQYASYPYT |
| 3P2-66 | (SEQ ID NO: | ID NO: | (SEQ ID NO: |
| | 1210) | 599) | 1103) |
| BP003T | QSIVHSNG | KVS (SEQ | FQGSHVPPT |
| 3P2-67 | NTY (SEQ | ID NO: | (SEQ ID NO: |
| | ID NO: | 1062) | 966) |
| | 1299) | | |
| BP003T | QSIVHSNG | KVS (SEQ | FQGSHVPRT |
| 3P2-68 | NTY (SEQ | ID NO: | (SEQ ID NO: |
| | ID NO: | 1062) | 967) |
| | 1299) | | |
| BP003T | QSIVHNNG | KVS (SEQ | FQGSYVPRT |
| 3P2-69 | NTY (SEQ | ID NO: | (SEQ ID NO: |
| | ID NO: | 1062) | 971) |
| | 1298) | | |
| BP003T | ENIYSY | NAK (SEQ | QHHYDTPY |
| 3P2-7 | (SEQ ID NO: | ID NO: 28) | T (SEQ ID |
| | 930) | | NO: 30) |
| BP003T | QSIVHSNG | KVS (SEQ | FQGSHVPPT |
| 3P2-70 | NTY (SEQ | ID NO: | (SEQ ID NO: |
| | ID NO: | 1062) | 966) |
| | 1299) | | |
| BP003T | QSIVHSNG | KVS (SEQ | FQGSHVPPT |
| 3P2-71 | NTY (SEQ | ID NO: | (SEQ ID NO: |
| | ID NO: | 1062) | 966) |
| | 1299) | | |
| BP003T | QSIVHSNG | KVS (SEQ | FQGSHVPTF |
| 3P2-72 | NTY (SEQ | ID NO: | (SEQ ID NO: |
| | ID NO: | 1062) | 968) |
| | 1299) | | |
| BP003T | QSIVHSNG | KVS (SEQ | FQGSHVPPT |
| 3P2-73 | NTY (SEQ | ID NO: | (SEQ ID NO: |
| | ID NO: | 1062) | 966) |
| | 1299) | | |
| BP003T | QSIVHSNG | KVS (SEQ | FQGSHVPPT |
| 3P2-74 | NTY (SEQ | ID NO: | (SEQ ID NO: |
| | ID NO: | 1062) | 966) |
| | 1299) | | |
| BP003T | QDVSTA | SAS (SEQ | QQHYSTPYT |
| 3P2-75 | (SEQ ID NO: | ID NO: 13) | (SEQ ID NO: |
| | 1209) | | 1265) |
| BP003T | ENIYSY | NAK (SEQ | QHHYDTPY |
| 3P2-8 | (SEQ ID NO: | ID NO: 28) | T (SEQ ID |
| | 930) | | NO: 30) |
| BP003T | DNIYNY | NAK (SEQ | QHHYGSPW |
| 3P2-9 | (SEQ ID NO: | ID NO: 28) | T (SEQ ID |
| | 833) | | NO: 1215). |

20. The method of claim 19, cancer or an autoimmune-related disease or disorder.

21. The method of claim 19, wherein the subject suffers from an autoimmune-related disease or disorder.

22. A monoclonal CD25 antibody which binds to human CD25 comprising at least one of:
a. the antibody comprises the amino acid sequence of CDRH1, CDRH2, and CDRH3 of any one of the combinations presented in SEQ ID NOS: 4, 6, 8, 19, 21, 23, 46, 48, 151-183, 224-258, 297-344, 482-492, 506-515, 531-557, 601-608, 611, 612, 614, 615, 617, 619, 691-697, 973, 977, 982, 984-987, 989-997, 1003, 1004, 1012-1016, 1020-1029, 1035, 1165, 1377-1379, 1395, 1416-1420, 1442-1445 and Table 6 and the amino acid sequence of CDRL1, CDRL2, and CDRL3 of any one of the combinations presented in SEQ ID NOS: 12, 13, 15, 27, 28, 30, 36, 42, 52, 54, 599, 600, 618, 833, 839, 924, 925, 927-930, 932-935, 944, 957, 966-972, 975, 976, 979-981, 983, 998-1002, 1036-1038, 1040, 1057-1063, 1081, 1092-1107, 1115, 1116, 1122, 1136, 1142, 1198-1202, 1204-1217, 1242-1244, 1246-1287, 1289-1296, 1298-1311, 1344-1346, 1360-1362, 1365, 1369-1376, 1380-1386, 1391, 1450, 1461-1466, 1475, 1476, 1495 and Table 7;
b. the antibody does not disrupt the binding of the IL-2 ligand to the alpha chain of the IL-2 receptor (CD25), and binds to a different epitope than to which 7G7B6 binds;
c. the antibody does not disrupt the binding of the IL-2 ligand to the alpha chain of the IL-2 receptor (CD25), but does disrupt the trimerization of the beta, gamma, and alpha (CD25) chains of the IL-2 receptor;
d. the antibody disrupts the binding of the IL-2 ligand to the alpha (CD25), beta, and/or gamma chains of the IL-2 receptor, and binds to a different epitope than to which daclizumab or baciliximab bind;
e. the antibody exhibits a higher affinity of binding to CD25 at pH lower than 7.4, when compared to the affinity of binding to CD25 at a pH of 7.4;
f. the antibody comprises the amino acid sequence of any one of the variable heavy chain sequences presented in SEQ ID NOS: 2, 4, 6, 8, 17, 19, 21, 23, 32, 38, 44, 46, 48, 56, 60-107, 114, 157, 190, 195, 230, 235, 251, 265, 269, 398, 413, 421, 422, 424, 425, 431-460, 473, 601, 603-606, 608, 611, 615, 617, 619, 868, 869, 936-940, 942, 943, 945-952, 954, 955, 973, 977, 984, 986, 987, 992, 995, 997, 1003-1008, 1010-1015, 1017, 1018, 1020, 1021, 1023, 1025-1027, 1029-1035, 1074, 1075, 1118, 1119, 1123-1128, 1137-1139, 1165-1172, 1174-1179, 1181-1185, 1187, 1188, 1190-1197, 1218, 1220, 1222, 1223-1241, 1313-1318, 1321-1343, 1387-1389, 1395, 1416, 1417, 1419, 1420, 1442-1445, 1453-1455, 1448, 1474, 1496 and Tables 1A, 1C, 1E, 1G, 1I, 1K, 2A, 2B, 2C, 4A, humanized versions thereof, or an amino acid sequence comprising at least a 80%, at least a 85%, at least a 90%, or at least a 95% sequence identity thereto;
g. the antibody comprises the amino acid sequence of any one of the variable light chain sequences presented in SEQ ID NOS: 10, 12, 13, 15, 25, 27, 28, 30, 34, 36, 40, 50, 52, 54, 59, 599, 618, 700, 701, 703, 705, 707, 709, 711, 712, 713, 715, 717-720, 722, 724, 725, 727-732, 734-740, 742, 744, 745, 747, 749-752, 754-757, 759, 760, 762, 765-767, 769, 770, 773, 775-778, 780, 781, 783, 785, 787, 789, 791, 793, 794, 795, 797, 799, 801, 803, 804, 806, 807, 809-811, 813, 815, 816, 818, 821-825, 827-833, 835, 837, 838, 841, 842, 844, 846, 848, 849, 851, 853, 854, 857, 858-861, 864, 865, 870-880, 882, 884, 886, 888, 889, 890, 892, 894, 895, 897, 899, 900-903, 905-909, 912, 914, 915, 917-922, 926, 928, 930, 944, 958, 959, 961, 963, 966-968, 971, 974, 978, 1002, 1057, 1062, 1070-1072, 1076, 1085, 1088, 1091, 1097, 1103, 1105, 1106, 1109, 1110, 1112, 1117, 1132, 1151, 1154, 1157-1159, 1164, 1198, 1200-1202, 1206-1210, 1214-1216, 1246, 1248, 1253, 1255, 1258, 1265, 1271, 1272, 1281, 1283-1285, 1287, 1290, 1298, 1299, 1306, 1309, 1345, 1347-1350, 1352, 1353, 1355-1357, 1359, 1366, 1371, 1375, 1386, 1396-1402, 1404, 1408, 1413, 1414, 1423-1425, 1427, 1429, 1430, 1432, 1433, 1436, 1447, 1449, 1450, 1470, 1471, 1475-1478, 1481-1483, 1486-1491, 1495 and Tables 1B, 1D, 1F, 1H, 1J, 1L, 3A, 3B, 3C, 4B, humanized versions thereof, or an amino acid sequence comprising at least a 80%, at least a 85%, at least a 90%, or at least a 95% sequence identity thereto;
h. the VH of the antibody comprises any one of the amino acid sequence of CDRH1, CDRH2, and CDRH3 as presented in SEQ ID NOS: 4, 6, 8, 19, 21, 23, 46, 48, 114, 151-183, 190, 195, 224-258, 265, 269, 297-344, 398, 413, 421, 422, 424, 425, 431, 473, 482-492, 506-515, 531-557, 601-608, 611, 612, 614, 615, 617, 619, 691-697, 936-940, 942, 943, 945-952, 954, 955, 973, 977, 982, 984-987, 989-997, 1003-1008, 1010-1016, 1017, 1018, 1020-1029, 1025-1035, 1321-1326, 1336-1343, 1377-1379, 1074, 1075, 1118, 1119, 1123-1128, 1137-1139, 1165-1172, 1174-1179, 1181-1185, 1187, 1188, 1190-1197, 1218, 1220, 1223-1228, 1239-1241, 1313-1315, 1387-1389, 1395, 1416-1420, 1442-1445, 1448, 1453-1455, 1474, 1496 and Tables 1A, 1C, 1E, 1G, 1I, 1K, 2A, 2B, 2C, 4A, 5A, and 6; and
i. the VL of the CD25 antibody comprises any one of the amino acid sequence of CDRL1, CDRL2, and CDRL3 as presented in SEQ ID NOS: 12, 13, 15, 27, 28, 30, 36, 42, 52, 54, 599, 600, 618, 700, 711, 713, 718-720, 729-732, 734, 738, 740, 744, 757, 760, 766, 770, 773, 793, 807, 816, 821, 828, 830-833, 837, 839, 854, 858, 865, 870-874, 889, 890, 894, 899, 900, 909, 915, 918, 924-930, 932-935, 944, 957-959, 961, 963, 966-972, 974-976, 978-981, 983, 998-1002, 1036-1038, 1040, 1057-1063, 1070-1072, 1076, 1081, 1085, 1088, 1091-1107, 1109, 1110, 1112, 1115-1117, 1122, 1132, 1136, 1142, 1151, 1154, 1157-1159, 1164, 1198-1202, 1204-1217, 1242-1244, 1246-1287, 1289-1296, 1298-1311, 1344-1350, 1352, 1353, 1355-1357, 1359, 1360-1362, 1365, 1366, 1369-1376, 1380-1386, 1391, 1396-1402, 1404, 1408, 1413, 1414, 1423-1425, 1427, 1429, 1430, 1432, 1433, 1436, 1447, 1449, 1450, 1461-1478, 1481-1483, 1486-1491, 1495 and Tables 1B, 1D, 1F, 1H, 1J, 1L, 3A, 3B, 3C, 4B, 5B and 7.


SEQUENCE LISTING (1501 sequences)

SEQ ID NO: 1 - DNA, 343 bp, Mus musculus
  source 1..343: mol_type = genomic DNA
catgtgcaactgcagcagtctggagctgagctggtaaggcctgggacttcagtgaagatatcctgcaaggcttctggctacaccttcactaactactggctaggttgggtaaagcagaggcctggacatggacttgagtggattggagatatttaccctggaggtggttatactaactacaatgagaagttcaagggcaaggccacactgactgcagacacatcctccagcactgcctacatgcagctcagtagcctgacatctgaggactctgctgtctatttctgtgcaagagttactccggcttcctggggccaaggcaccagtctcacagtctcctcgg

SEQ ID NO: 2 - protein, 114 aa, Mus musculus
HVQLQQSGAELVRPGTSVKISCKASGYTFTNYWLGWVKQRPGHGLEWIGDIYPGGGYTNYNEKFKGKATLTADTSSSTAYMQLSSLTSEDSAVYFCARVTPASWGQGTSLTVSS

SEQ ID NO: 3 - DNA, 24 bp, Mus musculus
  source 1..24: mol_type = genomic DNA
ggctacaccttcactaactactgg

SEQ ID NO: 4 - protein, 8 aa, Mus musculus
GYTFTNYW

SEQ ID NO: 5 - DNA, 24 bp, Mus musculus
  source 1..24: mol_type = genomic DNA
atttaccctggaggtggttatact

SEQ ID NO: 6 - protein, 8 aa, Mus musculus
IYPGGGYT

SEQ ID NO: 7 - DNA, 21 bp, Mus musculus
  source 1..21: mol_type = genomic DNA
gcaagagttactccggcttcc

SEQ ID NO: 8 - protein, 7 aa, Mus musculus
ARVTPAS

SEQ ID NO: 9 - DNA, 322 bp, Mus musculus
  source 1..322: mol_type = genomic DNA
gatattgtgatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatccgtacacgttcggaggggggaccaagctggaaatgaaac

SEQ ID NO: 10 - protein, 107 aa, Mus musculus
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEMK

SEQ ID NO: 11 - DNA, 18 bp, Mus musculus
  source 1..18: mol_type = genomic DNA
cagaatgtgggtactaat

SEQ ID NO: 12 - protein, 6 aa, Mus musculus
QNVGTN

SEQ ID NO: 13 - skipped

SEQ ID NO: 14 - DNA, 27 bp, Mus musculus
  source 1..27: mol_type = genomic DNA
cagcaatataacagctatccgtacacg

SEQ ID NO: 15 - protein, 9 aa, Mus musculus
QQYNSYPYT

SEQ ID NO: 16 - DNA, 346 bp, Mus musculus
  source 1..346: mol_type = genomic DNA
cagatccaactgcagcagcctggggctgagctggtgaggcctggggtttcactgaaaatttcctgcaagggttctggctacacattcactgattatgctatgcactgggtgaggcagagtcatgcaaagagtctagagtggattggagttattagtacttactctggtgatgctatctacaaccagaagttcaagggcaaggccacaatgactgtcgacaaatcctccagcacagcctatctggaacttgccagactgacatctgacgattctgccatctattactgtgcaagaggggtaacttttgactactggggccaaggcaccactgtcacagtctcctcgg

SEQ ID NO: 17 - protein, 115 aa, Mus musculus
QIQLQQPGAELVRPGVSLKISCKGSGYTFTDYAMHWVRQSHAKSLEWIGVISTYSGDAIYNQKFKGKATMTVDKSSSTAYLELARLTSDDSAIYYCARGVTFDYWGQGTTVTVSS

SEQ ID NO: 18 - DNA, 24 bp, Mus musculus
  source 1..24: mol_type = genomic DNA
ggctacacattcactgattatgct

SEQ ID NO: 19 - protein, 8 aa, Mus musculus
GYTFTDYA

SEQ ID NO: 20 - DNA, 24 bp, Mus musculus
  source 1..24: mol_type = genomic DNA
attagtacttactctggtgatgct

SEQ ID NO: 21 - protein, 8 aa, Mus musculus
ISTYSGDA

SEQ ID NO: 22 - DNA, 24 bp, Mus musculus
  source 1..24: mol_type = genomic DNA
gcaagaggggtaacttttgactac

SEQ ID NO: 23 - protein, 8 aa, Mus musculus
ARGVTFDY

SEQ ID NO: 24 - DNA, 322 bp, Mus musculus
  source 1..322: mol_type = genomic DNA
gacatccagatgacacagactacatcctccctgtctgcctctctgggagacagagtcaccatcacttgcagggcaagtcaggacattagcaattatttagaatggtatcagcagaaacagggaaaatctcctcagctcctggtctataatgcaaaaaccttagcagaaggtgtgccatcaaggttcagtggcagtggatcaggcacacagttttctctgaagatcaacagcctgcagcctgaagattttgggagttattactgtcaacatcattatgatactccgtacacgttcggaggggggaccaagctggaaataaaac

SEQ ID NO: 25 - protein, 107 aa, Mus musculus
DIQMTQTTSSLSASLGDRVTITCRASQDISNYLEWYQQKQGKSPQLLVYNAKTLAEGVPSRFSGSGSGTQFSLKINSLQPEDFGSYYCQHHYDTPYTFGGGTKLEIK

SEQ ID NO: 26 - DNA, 18 bp, Mus musculus
  source 1..18: mol_type = genomic DNA
caggacattagcaattat

SEQ ID NO: 27 - protein, 6 aa, Mus musculus
QDISNY

SEQ ID NO: 28 - skipped

SEQ ID NO: 29 - protein, 27 aa, Mus musculus
CAACATCATTATGATACTCCGTACACG

SEQ ID NO: 30 - protein, 9 aa, Mus musculus
QHHYDTPYT

SEQ ID NO: 31 - DNA, 343 bp, Mus musculus
  source 1..343: mol_type = genomic DNA
caggtgcaaatgcagcagtctggagctgagctggtaaggcctgggacttcagtgaagatatcctgcaaggcttctggctacaccttcactaactactggctaggttgggtaaagcagaggcctggacatggacttgagtggattggagatatttaccctggaggtggttatactaactacaatgagaagttcaagggcaaggccacactgactgcagacacatcctccagcactgcctacatgcagctcagtagcctgacatctgaggactctgctgtctatttctgtgcaagagttactccggcttcctggggccaaggcaccactctcacagtctcctcgg

SEQ ID NO: 32 - protein, 114 aa, Mus musculus
QVQMQQSGAELVRPGTSVKISCKASGYTFTNYWLGWVKQRPGHGLEWIGDIYPGGGYTNYNEKFKGKATLTADTSSSTAYMQLSSLTSEDSAVYFCARVTPASWGQGTTLTVSS

SEQ ID NO: 33 - DNA, 322 bp, Mus musculus
  source 1..322: mol_type = genomic DNA
gatatccagatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatccgtggacgttcggtggaggcaccaagctggaaatcaaac

SEQ ID NO: 34 - protein, 107 aa, Mus musculus
DIQMTQSQKFMSTSVGDRVSVTCKASQNVGTNVAWYQQKPGQSPKALIYSASYRYSGVPDRFTGSGSGTDFTLTISNVQSEDLAEYFCQQYNSYPWTFGGGTKLEIK

SEQ ID NO: 35 - DNA, 27 bp, Mus musculus
  source 1..27: mol_type = genomic DNA
cagcaatataacagctatccgtggacg

SEQ ID NO: 36 - protein, 9 aa, Mus musculus
QQYNSYPWT

SEQ ID NO: 37 - DNA, 343 bp, Mus musculus
  source 1..343: mol_type = genomic DNA
cagatccaactgcagcagtctggagctgagctggtaaggcctgggacttcagtgaagatatcctgcaaggcttctggctacaccttcactaactactggctaggttgggtaaagcagaggcctggacatggacttgagtggattggagatatttaccctggaggtggttatactaactacaatgagaagttcaagggcaaggccacactgactgcagacacatcctccagcactgcctacatgcagctcagtagcctgacatctgaggactctgctgtctatttctgtgcaagagttactccggcttcctggggccaaggcaccactctcacagtctctgcgg

SEQ ID NO: 38 - protein, 114 aa, Mus musculus
QIQLQQSGAELVRPGTSVKISCKASGYTFTNYWLGWVKQRPGHGLEWIGDIYPGGGYTNYNEKFKGKATLTADTSSSTAYMQLSSLTSEDSAVYFCARVTPASWGQGTTLTVSA

SEQ ID NO: 39 - DNA, 322 bp, Mus musculus
  source 1..322: mol_type = genomic DNA
gacattctgctgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatcctctcacgttcggaggggggaccaagctggaaataaaac

SEQ ID NO: 40 - protein, 107 aa, Mus musculus
DILLTQSQKFMSTSVGDRVSVTCKASQNVGTNVAWYQQKPGQSPKALIYSASYRYSGVPDRFTGSGSGTDFTLTISNVQSEDLAEYFCQQYNSYPLTFGGGTKLEIK

SEQ ID NO: 41 - DNA, 27 bp, Mus musculus
  source 1..27: mol_type = genomic DNA
cagcaatataacagctatcctctcacg

SEQ ID NO: 42 - protein, 9 aa, Mus musculus
QQYNSYPLT

SEQ ID NO: 43 - DNA, 345 bp, Mus musculus
  source 1..345: mol_type = genomic DNA
caggtccaactgcagcagtctggggctgagctggtgaggcctggggtctcagtgaagatttcctgcaagggttctggctacacattcactgattatgctatgcactgggtgaagcagagtcatgcaaagagtctagagtggattggagttattagtacttactctggtgatgttagttacaaccagaagttcaagggcaaggccacaatgactgtcgacaaatcctccagcacagcctatatggaacttgccagactgacatctgaggattctgccatctattactgtgcaagaggggtaacttttgactcctggggccaaggcaccacggtcaccgtctcctcg

SEQ ID NO: 44 - protein, 115 aa, Mus musculus
QVQLQQSGAELVRPGVSVKISCKGSGYTFTDYAMHWVKQSHAKSLEWIGVISTYSGDVSYNQKFKGKATMTVDKSSSTAYMELARLTSEDSAIYYCARGVTFDSWGQGTTVTVSS

SEQ ID NO: 45 - DNA, 24 bp, Mus musculus
  source 1..24: mol_type = genomic DNA
attagtacttactctggtgatgtt

SEQ ID NO: 46 - protein, 8 aa, Mus musculus
ISTYSGDV

SEQ ID NO: 47 - DNA, 24 bp, Mus musculus
  source 1..24: mol_type = genomic DNA
gcaagaggggtaacttttgactcc

SEQ ID NO: 48 - protein, 8 aa, Mus musculus
ARGVTFDS

SEQ ID NO: 49 - DNA, 322 bp, Mus musculus
  source 1..322: mol_type = genomic DNA
gacatccagatgacacagtctccagcctccctatctgcatctgtgggagaaactgtcaccatcacatgtcgagcaagtgagaatagttacagttatttagaatggtatcagcagaaacagggaaaatctcctcagctcctggtctataatgcaaaaactttagcagaaggtgtgccatcaaggttcagtggcagtggatcaggcacacagttttctctgaagatcaacagcctgcagcctgaagattttgggacttattactgtcaacatcattatggtactccgtacacgttcggaggggggaccaagctggaaataaaac

SEQ ID NO: 50 - protein, 107 aa, Mus musculus
DIQMTQSPASLSASVGETVTITCRASENSYSYLEWYQQKQGKSPQLLVYNAKTLAEGVPSRFSGSGSGTQFSLKINSLQPEDFGTYYCQHHYGTPYTFGGGTKLEIK

SEQ ID NO: 51 - DNA, 18 bp, Mus musculus
  source 1..18: mol_type = genomic DNA
gagaatagttacagttat

SEQ ID NO: 52 - protein, 6 aa, Mus musculus
ENSYSY

SEQ ID NO: 53 - DNA, 27 bp, Mus musculus
  source 1..27: mol_type = genomic DNA
caacatcattatggtactccgtacacg

SEQ ID NO: 54 - protein, 9 aa, Mus musculus
QHHYGTPYT

SEQ ID NO: 55 - DNA, 346 bp, Mus musculus
  source 1..346: mol_type = genomic DNA
caggtccaactgcagcagcctggggctgagctggtgaggcctggggtttcactgaagatttcctgcaagggttctggctacacattcactgattatgctatgcactgggtgaggcagagtcatgcaaagagtctagagtggattggagttattagtacttactctggtgatgctctctacaaccagaagttcaagggcaaggccacaatgactgtcgacaaatcctccagcacagcctatctggaacttgccagactgacatctgaggattctgccatctattactgtgcgcgaggggtaacttttgactactggggccaaggcaccactctcacagtctcctcgg

SEQ ID NO: 56 - protein, 115 aa, Mus musculus
QVQLQQPGAELVRPGVSLKISCKGSGYTFTDYAMHWVRQSHAKSLEWIGVISTYSGDALYNQKFKGKATMTVDKSSSTAYLELARLTSEDSAIYYCARGVTFDYWGQGTTLTVSS

SEQ ID NO: 57 - DNA, 24 bp, Mus musculus
  source 1..24: mol_type = genomic DNA
gcgcgaggggtaacttttgactac

SEQ ID NO: 58 - DNA, 322 bp, Mus musculus
  source 1..322: mol_type = genomic DNA
gatgttgtgatgacccagtctccagcctccctatctgcatctgtgggagaaactgtcaccatcacatgtcgagcaagtgagaatagttacagttatttagaatggtatcagcagaaacagggaaaatctcctcagctcctggtctataatgcaaaaactttagcagaaggtgtgccatcaaggttcagtggcagtggatcaggcacacagttttctctgaagatcaacagcctgcagcctgaagattttgggacttattactgtcaacatcattatggtactccgtacacgttcggaggggggaccaagctggaaatgaaac

SEQ ID NO: 59 - protein, 107 aa, Mus musculus
DVVMTQSPASLSASVGETVTITCRASENSYSYLEWYQQKQGKSPQLLVYNAKTLAEGVPSRFSGSGSGTQFSLKINSLQPEDFGTYYCQHHYGTPYTFGGGTKLEMK

SEQ ID NO: 60 - protein, 119 aa, Mus musculus
DVKLVESGGGLVKPGGSLKLSCAASGFTFSSYAMSWVRQTPEKRLEWVASISSGGSTYYPDSVKGRFTISRDNARNILYLQMSSLRSEDTAMYYCARGEIWGKAWFAYWGQGTLVTVSA

SEQ ID NO: 61 - protein, 113 aa, Mus musculus
QVQMKQSGPELKKPGETVKISCKASGYTLTDYSMHWVKQAPGKGLKWMGWINTETGEPTYADDFKGRFAFSLETSASTAYLQINNLKNEDTATYFCAWGNHYWGQGTTVTVSS

SEQ ID NO: 62 - protein, 118 aa, Mus musculus
QIQLQQSGPELVKPGASVKMSCKASGYTFTSYVMHWVKQKPGQGLEWIGYINPYNDGTKYNEKFKGKATLTSDKSSSTAYMELSSLTSEDSAVYYCARDGYYVGPAYWGQGTLVTVSA

SEQ ID NO: 63 - protein, 117 aa, Mus musculus
EVLLVESGGGLVKPGGSLKLSCAASGFAFSSYDMSWVRQTPEKRLEWVAYISSGGGSTYYPDTVKGRFTISRDNAKNTLYLQMSSLKSEDTAMYYCARNYRSWFAYWGQGTLVTVSA

SEQ ID NO: 64 - protein, 119 aa, Mus musculus
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARKGSLTGVLAYWGQGTLVTVSA

SEQ ID NO: 65 - protein, 118 aa, Mus musculus
QVQLQQSGAELVRPGASVKLSCKASGYIFTNYWMNWVKQRPGQGLEWIGMIDPSDSETHYNQMSKDKATLTVDKSSSTAYMQLSSLTSEDSAVYYCARRGLRAWFAYWGQGTLVTVSA

SEQ ID NO: 66 - protein, 121 aa, Mus musculus
QVQLQQPGAELAKPGASVKMSCKASGYTFTSYWMHWVKQRPGQGLEWIGYINPSTGYTEYNQKFKDKATLTADKSSSTAYMQLSSLTSEDSAVYYCARLDYYGSSRGFAYWGQGTLVTVSA

SEQ ID NO: 67 - protein, 114 aa, Mus musculus
QVQLQQSGAELVKPGASVKLSCKASGYTFTSYYMYWVKQRPGQGLEWIGEINPSNGGTNFNEKFKSKATLTVDKSSSTAYMQLSSLTSEDSAVYYCTNGGGWYWGQGTTVTVSS

SEQ ID NO: 68 - protein, 120 aa, Mus musculus
QIQLVQSGPELKKPGETVKISCKASGYTFTNYGMNWVKQTPGKGLKWMGWINTYTGEPTYADDFKGRFAFSLETSASTAYLQINNLKNEDTATYFCASYYDSTYVGFAYWGQGTLVTVSA

SEQ ID NO: 69 - protein, 118 aa, Mus musculus
EVLLVESGGGLVQPGGSLKLSCATSGFTFSDYYMYWVRQTPEKRLEWVAYISNGGGSTYYPDTVKGRFTISRDNAKNTLYLQMSRLKSEDTAMYYCASPLGYDGFAYWGQGTLVTVSA

SEQ ID NO: 70 - protein, 114 aa, Mus musculus
QVQMKQSGPGLVAPSQSLSITCTVSGFSLTSYGVHWVRQPPGKGLEWLGVIWAGGSTNYNSALMSRLSISKDNSKSQVFLKMNSLQTDDTAMYYCARGAYFDYWGQGTTVTVSS

SEQ ID NO: 71 - protein, 121 aa, Mus musculus
QVQLQQSGAELVRPGASVKLSCKASGYTFTSYWMNWVKQRPEQGLEWIGRIDPYDSETHYNQKFKDKAILTVDKSSSTAYMQLSSLTSEDSAVYYCARSPAYYGNLWFAYWGQGTLVTVSA

SEQ ID NO: 72 - protein, 114 aa, Mus musculus
QVQLQQSGAELARPGASVKMSCKASGYTFTSYTIHWVKQRPGQGLEWIGYINPSSGYTNYNQKFKDKATLTADKSSSTAYMQLSSLTSEDSAVYYCARWDGAYWGQGTPVTVSS

SEQ ID NO: 73 - protein, 118 aa, Mus musculus
DVKLVESGGGLVKPGGSLKLSCAASGFTFSSYAMSWVRQFPEKRLEWVAEISSGGSYTYYPDTVTGRFTISRDNAKNTLYLEMSSLRSEDTAMYYCARGGMITPFAYWGQGTLVTVSS

SEQ ID NO: 74 - protein, 123 aa, Mus musculus
QVILKESGPGILQPSQTLSLTCSFSGFSLSTSGMSVGWIRQPSGKGLEWLAHIWWNDDKYYNPALKSRLTISKDTSNNQVFLKIASVVTADTATYYCARIGGNDGYYWYFDVWGAGTTVTVSS

SEQ ID NO: 75 - protein, 115 aa, Mus musculus
EVKIEESGGGLVQPGGSMKLSCAASGFTFSDAWMDWVRQSPEKGLEWVAEIRSKANNHATYYAESVKGRFTISRDDSKSSVYLQMNSLRAEDTGIYYCTPQFAYWGQGTTVTVSS

SEQ ID NO: 76 - protein, 115 aa, Mus musculus
EVMLVESGGGLVQPGGSLKLSCAASGFTFSSYGMSWVRQTPDKRLELVATINSNGGSTYYPDSVKGRFTISRDNAKNTLYLQMSSLKSEDTAMYYCASHYDEGYWGQGTSLTVSS

SEQ ID NO: 77 - protein, 114 aa, Mus musculus
EVKLEESGGGLVQPGGSMKLSCVASGFTFSNYWMNWVRQSPEKGLEWVAEIRLKSNNYATHYAESVKGRFTISRDDSKSSVYLQMNNLRAEDTGIYYCTGSDYWGQGTTVTVSS

SEQ ID NO: 78 - protein, 120 aa, Mus musculus
QVQLQQSGPELVKPGASVKMSCKASGDTFSSYVIHWVKQKPGQGLEWIGYFNPYSDDIKYNEKFKGKATLTSDKSSSTAYMELSSLTSEDSAVYYCGSGYDGYYDWFACWGQGTLVTVSA

SEQ ID NO: 79 - protein, 116 aa, Mus musculus
QIQLAQSGPELKKPGETVKISCKASGYSFTKNGMNWVKQAPGKGLKWMGWINTYTGEPTYADDFKGRFAFSLETSASTAYLQINNLKNEDTATYFCAREPKTLDYWGQGTTVTVSS

SEQ ID NO: 80 - protein, 115 aa, Mus musculus
QVQLQQSGAELVKPGAPVKLSCKASGYTFTSYWMNWVKQRPGRGLEWIGRIDPSDSETHYNQKFKDKATLTVDKSSSTAYIQLSSLTSEDSAVYYCANWAWFAYWGQGTLVTVSA

SEQ ID NO: 81 - protein, 118 aa, Mus musculus
QMQLKESGTELVRPGASVKLSCKASGYTFNSHWMNWVKQRPEQGLEWIGKIDPYDSETHYNQKFKDKAILTVDKSSSTAYMQLSSLTSEDSAVYYCARPYDYDGFAYWGQGTLVTVSA

SEQ ID NO: 82 - protein, 116 aa, Mus musculus
QIQLQQSGAELVRPGASVKISCKAFGYTFTNHHINWVKQRPGQGLDWIGYINPYNDYTSYNQKFKGKATLTVDKSSSTAYMELSSLTSEDSAVYYCADGDYYFDYWGQGTSLTVSS

SEQ ID NO: 83 - protein, 120 aa, Mus musculus
DVLLVESGGDLVKPGGSLKLSCVVSGLTFSSYGMSWVRQTPDKRLEWVATISSGGSYIYYVDSVKGRFTISRDNAKNTLYLQMSSLKSEDTAIYYCARQDDGYYRIFDYWGQGTTLTVSS

SEQ ID NO: 84 - protein, 119 aa, Mus musculus
EVKIEESGGGLVQPGGSMKLSCAASGFTFNDAWMDWVRQSPEKGLEWVAEIRSKANNHATYYAESVKGRFTISRDDSQSSVYLQMNSLRTENTGIYYCTNYGSNPLDYWGQGTTLTIPS

SEQ ID NO: 85 - protein, 116 aa, Mus musculus
QVTLKESGPGLVAPSQSLSITCTVSGFSLTSYGVHWVRQPPGKGLEWLGVIWAGGSTNYNSALMSRLGISKDNSKSQVFLKMNSLQTDESAFYYCAREGTGPWFAYWGQGTTAPSP

SEQ ID NO: 86 - protein, 115 aa, Mus musculus
DVQLQQSGPDLVKPSQSLSLTCTVTGYSITSGYSWHWIRQFPGNKLEWMGYIHYSGSTNYNPSLKSRISITRDTSKNQFFLQLNSVTTEGTATYYCARDPPFAYWGQGTLVTVSA

SEQ ID NO: 87 - protein, 118 aa, Mus musculus
QVQLQQSGAELVKPGASVRLSCKASGYTFTSYWMHWVKQRPGQGLEWIGEIDPSDSYTNYNQKFKGKATLAVDKSSSTAYMQLSSLTSEDSAVYYCAREEITAWFAYWGQGTLVTVSA

SEQ ID NO: 88 - protein, 119 aa, Mus musculus
QVQLQQPGAELVRPGASVKLSCKALGYTFTDYEMHWVKQTPVHGLEWIGAIHPGSGGTAYNQKFKGKATLTADKSSSTAYMELSSLTSEDSAVYYCTRNGNGNWYFDVWGAGTTLTVSS

SEQ ID NO: 89 - protein, 120 aa, Mus musculus
QVQLQQSGAELVRPGSSVKISCKASGYAFSSYWMNWVKQRPGQGLEWIGQIYPGDGDTYYNGKFKDKATLTADKSSSTAYMHLSSLTSEDSAVYFCARSGYRYDAVFAYWGPGTLVTVSA

SEQ ID NO: 90 - protein, 114 aa, Mus musculus
EVQLLESGGGLVQPGGSLKLSCAASGFTFSSYGMSWVRQTPDKRLELVATINSNGGSTYYPDSVKGRFTISRDNAKNTLYLQMSSLKSEDTAMYYCARGGNPYLGQGTLVTVSA

SEQ ID NO: 91 - protein, 107 aa, Mus musculus
EVQLVETGGGLVQPGGSRGLSCEGSGFTFSGFWMSWVRQTPGKTLEWIGDINSDGSAINYAPSIKDRFTIFRDNDKSTLYLQMSNVRSEDTATYFCMRYGSSYWYFD

SEQ ID NO: 92 - protein, 117 aa, Mus musculus
EVKIEESGGGLVQPGGSMKLSCVASGFTFSSYWMSWVRQSPEKGLEWVAEIRLKSDNYATHYAESVKGKFTISRDDSKSRLYLQMNSLRAEDTGIYYCTRYYYGESWGQGTLVTVSS

SEQ ID NO: 93 - protein, 121 aa, Mus musculus
EVMLVESGGDLVKPGGSLKLSCAASGFTFSSYGMSWVRQTPDKRLEWVATISSGGSYTYYPDSVKGRFTISRDNAKNTLYLQMSSLKSEDTAMYYCARHYYDYDYWYFDVWGAGTTVTVSS

SEQ ID NO: 94 - protein, 122 aa, Mus musculus
QVQLQQPGSELVRPGASVKLSCKASGYTFTSYWMHWVKQRYGQGLEWIGNIYPGSGSTNYDEKFKSKGTLTVDTSSSTAYMHLSSLTSEDSAVYYCTRSGVEGLLHWYFDVWGAGTSLTVSS

SEQ ID NO: 95 - protein, 121 aa, Mus musculus
EVKIEESGGGLVQPGGSMKLSCVASGFTFSSYWMSWVRQSPEKGLEWVAEIRLKSDNYATHYAESVKGKFTISRDDSKSRLYLQMNSLRAEDTGIYYCTCDYDGGAWFAYWGQGTLVTVSA

SEQ ID NO: 96 - protein, 119 aa, Mus musculus
QIQLQQPGPELVRPGASVKMSCKASGYTFTSYWMHWVKQRPGQGLEWIGMIDPSNSETRLNQKFKDKATLNVDKSSNTACMQLSSLTSEDSAVYYCARCDGYYDGLDYWGQGTTLTVSS

SEQ ID NO: 97 - protein, 118 aa, Mus musculus
QVQLQQPGAELARPGASVKMSCKASGYTFTSYTIHWVKQRPGQGLEWIGYINPSSGYTNYNQKFKDKATLTADKSSSTAYMQLSSLTSEDSAVYYCAREGKNWYFDVWGAGTTVTVSS

SEQ ID NO: 98 - protein, 117 aa, Mus musculus
QIQLQQSGAELVRPGVSVKLSCKASGYTFTSYWMHWIKQRPEQGLERIGEINPSNGGTNYNEKFKSKATLTVDKSSSTAYMQLSSLTSEDSAVYYCARRIYRTLDYWGQGTTVTVSS

SEQ ID NO: 99 - protein, 118 aa, Mus musculus
EVQLQQSGAELVKPGASVKMSCKASGYTFTSYNMHWVKQTPGQGLEWIGVIYPGNGDTAYNQKFKGKATVTADRSSSTAYMQLSSLTSEDSAVYYCTRSGGNLWFAYWGQGTLVTVSA

SEQ ID NO: 100 - protein, 120 aa, Mus musculus
QIQLQQSGAELMKPGASVKISCKATGYTFSSYWIEWVKQRPGHGLEWIGEILPGSGSTNYNEKFKGKATFTADTSSNTAYMQLSSLTSEDSAVYYCARRTYYGNAWFAYWGQGTLVTVSA

SEQ ID NO: 101 - protein, 124 aa, Mus musculus
QVKLVESGGGLVQPGGSLRLSCATSGFTFTDYYMSWVRQPPGKALEWLGFIRNKANGYTTEYSASVKGRFTISRDNSQSILYLQMNTLRAEDSATYYCARDKRITTVEAWFAYWGQGTLVTVSA

SEQ ID NO: 102 - protein, 116 aa, Mus musculus
EVKLVETGGGLVKPGGSLKLSCAASGFTFSSYAMSWVRQTPEKRLEWVASISSGGSTYYPDSVKGRFTISRDNARNILYLQMSSLRSEDTAMYYCARGYGSSFAYWGQGTPVTVSS

SEQ ID NO: 103 - protein, 120 aa, Mus musculus
QVQLQQSGAELVKPGASVKLSCKASGYTFTSYYMYWVKQRPGQGLEWIGEINPSNSGTNFNEKFKSKATLTVDKSSSTAYMQLSSLTSEDSAVYYCTRGGDYDASWFAYWGQGTLVTVSA

SEQ ID NO: 104 - protein, 117 aa, Mus musculus
QVQMKESGPEPVKPGASVKMSCRASGYTLTDYVVSWVKQRLGQGLEWIGEIYPGSGSTYYNEKFKDKVTLTADTSSNTVHIQLSSLTSEDSAVYFCARRTARAFDYWGQGTTVTVSS

SEQ ID NO: 105 - protein, 122 aa, Mus musculus
EVKLVESGGGLVKPGGSLKLSCAASGFTFSSYAMSWVRQTPEKRLEWVATISSGGSYTYYPDSVKGRFTISRDNAKNTLYLQMSSLRSEDTAMYYCARRIGYDGGGSWFAYWGQGTLVTVSA

SEQ ID NO: 106 - protein, 117 aa, Mus musculus
EVKIVESGGGLVQPKGSLKLSCAASGFTFNTYAMNWVRQAPEKGLEWVARIRSKSNNYVTYYADSLKDRFTISRDDSQSMLYLQMNNLKTEDTCHVLLCERVRRCVLGPRDSGHCLC

SEQ ID NO: 107 - protein, 115 aa, Mus musculus
QVQLVETGGGLVRPGNSLKLSCVTSGFTFSNYRMHWLRQPPGKRLEWIAVITVKSDNYGANYAESVKGRFTISRDDSKSSVYLQMNRLREEDTATYYCSRWFAYWGQGTLVTVSA

SEQ ID NO: 108 - protein, 25 aa, Mus musculus
DVKLVESGGGLVKPGGSLKLSCAAS

SEQ ID NO: 109 - protein, 25 aa, Mus musculus
QVQMKQSGPELKKPGETVKISCKAS

SEQ ID NO: 110 - protein, 25 aa, Mus musculus
QIQLQQSGPELVKPGASVKMSCKAS

SEQ ID NO: 111 - protein, 25 aa, Mus musculus
EVLLVESGGGLVKPGGSLKLSCAAS

SEQ ID NO: 112 - protein, 25 aa, Mus musculus
QVQLQQSGAELVRPGTSVKVSCKAS

SEQ ID NO: 113 - protein, 25 aa, Mus musculus
QVQLQQSGAELVRPGASVKLSCKAS

SEQ ID NO: 114 - protein, 25 aa, Mus musculus
QVQLQQPGAELAKPGASVKMSCKAS

SEQ ID NO: 115 - protein, 25 aa, Mus musculus
QVQLQQSGAELVKPGASVKLSCKAS

SEQ ID NO: 116 - protein, 25 aa, Mus musculus
QIQLVQSGPELKKPGETVKISCKAS

SEQ ID NO: 117 - protein, 25 aa, Mus musculus
EVLLVESGGGLVQPGGSLKLSCATS

SEQ ID NO: 118 - protein, 25 aa, Mus musculus
QVQMKQSGPGLVAPSQSLSITCTVS

SEQ ID NO: 119 - protein, 25 aa, Mus musculus
QVQLQQSGAELARPGASVKMSCKAS

SEQ ID NO: 120 - protein, 25 aa, Mus musculus
QVILKESGPGILQPSQTLSLTCSFS

SEQ ID NO: 121 - protein, 25 aa, Mus musculus
EVKIEESGGGLVQPGGSMKLSCAAS

SEQ ID NO: 122 - protein, 25 aa, Mus musculus
EVMLVESGGGLVQPGGSLKLSCAAS

SEQ ID NO: 123 - protein, 25 aa, Mus musculus
EVKLEESGGGLVQPGGSMKLSCVAS

SEQ ID NO: 124 - protein, 25 aa, Mus musculus
QVQLQQSGPELVKPGASVKMSCKAS

SEQ ID NO: 125 - protein, 25 aa, Mus musculus
QIQLAQSGPELKKPGETVKISCKAS

SEQ ID NO: 126 - protein, 25 aa, Mus musculus
QVQLQQSGAELVKPGAPVKLSCKAS

SEQ ID NO: 127 - protein, 25 aa, Mus musculus
QMQLKESGTELVRPGASVKLSCKAS

SEQ ID NO: 128 - protein, 25 aa, Mus musculus
QIQLQQSGAELVRPGASVKISCKAF

SEQ ID NO: 129 - protein, 25 aa, Mus musculus
DVLLVESGGDLVKPGGSLKLSCVVS

SEQ ID NO: 130 - protein, 25 aa, Mus musculus
QVTLKESGPGLVAPSQSLSITCTVS

SEQ ID NO: 131 - protein, 25 aa, Mus musculus
DVQLQQSGPDLVKPSQSLSLTCTVT

SEQ ID NO: 132 - protein, 25 aa, Mus musculus
QVQLQQSGAELVKPGASVRLSCKAS

SEQ ID NO: 133 - protein, 25 aa, Mus musculus
QVQLQQPGAELVRPGASVKLSCKAL

SEQ ID NO: 134 - protein, 25 aa, Mus musculus
QVQLQQSGAELVRPGSSVKISCKAS

SEQ ID NO: 135 - protein, 25 aa, Mus musculus
EVQLLESGGGLVQPGGSLKLSCAAS

SEQ ID NO: 136 - protein, 25 aa, Mus musculus
EVQLVETGGGLVQPGGSRGLSCEGS

SEQ ID NO: 137 - protein, 25 aa, Mus musculus
EVKIEESGGGLVQPGGSMKLSCVAS

SEQ ID NO: 138 - protein, 25 aa, Mus musculus
EVMLVESGGDLVKPGGSLKLSCAAS

SEQ ID NO: 139 - protein, 25 aa, Mus musculus
QVQLQQPGSELVRPGASVKLSCKAS

SEQ ID NO: 140 - protein, 25 aa, Mus musculus
QIQLQQPGPELVRPGASVKMSCKAS

SEQ ID NO: 141 - protein, 25 aa, Mus musculus
QVQLQQPGAELARPGASVKMSCKAS

SEQ ID NO: 142 - protein, 25 aa, Mus musculus
QIQLQQSGAELVRPGVSVKLSCKAS

SEQ ID NO: 143 - protein, 25 aa, Mus musculus
EVQLQQSGAELVKPGASVKMSCKAS

SEQ ID NO: 144 - protein, 25 aa, Mus musculus
QIQLQQSGAELMKPGASVKISCKAT

SEQ ID NO: 145 - protein, 25 aa, Mus musculus
QVKLVESGGGLVQPGGSLRLSCATS

SEQ ID NO: 146 - protein, 25 aa, Mus musculus
EVKLVETGGGLVKPGGSLKLSCAAS

SEQ ID NO: 147 - protein, 25 aa, Mus musculus
QVQMKESGPEPVKPGASVKMSCRAS

SEQ ID NO: 148 - protein, 25 aa, Mus musculus
EVKLVESGGGLVKPGGSLKLSCAAS

SEQ ID NO: 149 - protein, 25 aa, Mus musculus
EVKIVESGGGLVQPKGSLKLSCAAS

SEQ ID NO: 150 - protein, 25 aa, Mus musculus
QVQLVETGGGLVRPGNSLKLSCVTS

SEQ ID NO: 151 - protein, 8 aa, Mus musculus
GFTFSSYA

SEQ ID NO: 152 - protein, 8 aa, Mus musculus
GYTLTDYS

SEQ ID NO: 153 - protein, 8 aa, Mus musculus
GYTFTSYV

SEQ ID NO: 154 - protein, 8 aa, Mus musculus
GFAFSSYD

SEQ ID NO: 155 - protein, 8 aa, Mus musculus
GYAFTNYL

SEQ ID NO: 156 - protein, 8 aa, Mus musculus
GYIFTNYW

SEQ ID NO: 157 - protein, 8 aa, Mus musculus
GYTFTSYW

SEQ ID NO: 158 - protein, 8 aa, Mus musculus
GYTFTSYY

SEQ ID NO: 159 - protein, 8 aa, Mus musculus
GYTFTNYG

SEQ ID NO: 160 - protein, 8 aa, Mus musculus
GFTFSDYY

SEQ ID NO: 161 - protein, 8 aa, Mus musculus
GFSLTSYG

SEQ ID NO: 162 - protein, 8 aa, Mus musculus
GYTFTSYT

SEQ ID NO: 163 - protein, 10 aa, Mus musculus
GFSLSTSGMS

SEQ ID NO: 164 - protein, 8 aa, Mus musculus
GFTFSDAW

SEQ ID NO: 165 - protein, 8 aa, Mus musculus
GFTFSSYG

SEQ ID NO: 166 - protein, 8 aa, Mus musculus
GFTFSNYW

SEQ ID NO: 167 - protein, 8 aa, Mus musculus
GDTFSSYV

SEQ ID NO: 168 - protein, 8 aa, Mus musculus
GYSFTKNG

SEQ ID NO: 169 - protein, 8 aa, Mus musculus
GYTFNSHW

SEQ ID NO: 170 - protein, 8 aa, Mus musculus
GYTFTNHH

SEQ ID NO: 171 - protein, 8 aa, Mus musculus
GLTFSSYG

SEQ ID NO: 172 - protein, 8 aa, Mus musculus
GFTFNDAW

SEQ ID NO: 173 - protein, 9 aa, Mus musculus
GYSITSGYS

SEQ ID NO: 174 - protein, 8 aa, Mus musculus
GYTFTDYE

SEQ ID NO: 175 - protein, 8 aa, Mus musculus
GYAFSSYW

SEQ ID NO: 176 - protein, 8 aa, Mus musculus
GFTFSGFW

SEQ ID NO: 177 - protein, 8 aa, Mus musculus
GFTFSSYW

SEQ ID NO: 178 - protein, 8 aa, Mus musculus
GYTFTSYN

SEQ ID NO: 179 - protein, 8 aa, Mus musculus
GYTFSSYW

SEQ ID NO: 180 - protein, 8 aa, Mus musculus
GFTFTDYY

SEQ ID NO: 181 - protein, 8 aa, Mus musculus
GYTLTDYV

SEQ ID NO: 182 - protein, 8 aa, Mus musculus
GFTFNTYA

SEQ ID NO: 183 - protein, 8 aa, Mus musculus
GFTFSNYR

SEQ ID NO: 184 - protein, 17 aa, Mus musculus
MSWVRQTPEKRLEWVAS

SEQ ID NO: 185 - protein, 17 aa, Mus musculus
MHWVKQAPGKGLKWMGW

SEQ ID NO: 186 - protein, 17 aa, Mus musculus
MHWVKQKPGQGLEWIGY

SEQ ID NO: 187 - protein, 17 aa, Mus musculus
MSWVRQTPEKRLEWVAY

SEQ ID NO: 188 - protein, 17 aa, Mus musculus
IEWVKQRPGQGLEWIGV

SEQ ID NO: 189 - protein, 17 aa, Mus musculus
MNWVKQRPGQGLEWIGM

SEQ ID NO: 190 - protein, 17 aa, Mus musculus
MHWVKQRPGQGLEWIGY

SEQ ID NO: 191 - protein, 17 aa, Mus musculus
MYWVKQRPGQGLEWIGE

SEQ ID NO: 192 - protein, 17 aa, Mus musculus
MNWVKQTPGKGLKWMGW

SEQ ID NO: 193 - protein, 17 aa, Mus musculus
MYWVRQTPEKRLEWVAY

SEQ ID NO: 194 - protein, 17 aa, Mus musculus
VHWVRQPPGKGLEWLGV

SEQ ID NO: 195 - protein, 17 aa, Mus musculus
MNWVKQRPEQGLEWIGR

SEQ ID NO: 196 - protein, 17 aa, Mus musculus
IHWVKQRPGQGLEWIGY

SEQ ID NO: 197 - protein, 17 aa, Mus musculus
MSWVRQFPEKRLEWVAE

SEQ ID NO: 198 - protein, 17 aa, Mus musculus
VGWIRQPSGKGLEWLAH

SEQ ID NO: 199 - protein, 17 aa, Mus musculus
MDWVRQSPEKGLEWVAE

SEQ ID NO: 200 - protein, 17 aa, Mus musculus
MSWVRQTPDKRLELVAT

SEQ ID NO: 201 - protein, 17 aa, Mus musculus
MNWVRQSPEKGLEWVAE

SEQ ID NO: 202 - protein, 17 aa, Mus musculus
IHWVKQKPGQGLEWIGY

SEQ ID NO: 203 - protein, 17 aa, Mus musculus
MNWVKQAPGKGLKWMGW

SEQ ID NO: 204 - protein, 17 aa, Mus musculus
MNWVKQRPGRGLEWIGR

SEQ ID NO: 205 - protein, 17 aa, Mus musculus
MNWVKQRPEQGLEWIGK

SEQ ID NO: 206 - protein, 17 aa, Mus musculus
INWVKQRPGQGLDWIGY

SEQ ID NO: 207 - protein, 17 aa, Mus musculus
MSWVRQTPDKRLEWVAT

SEQ ID NO: 208 - protein, 17 aa, Mus musculus
WHWIRQFPGNKLEWMGY

SEQ ID NO: 209 - protein, 17 aa, Mus musculus
MHWVKQRPGQGLEWIGE

SEQ ID NO: 210 - protein, 17 aa, Mus musculus
MHWVKQTPVHGLEWIGA

SEQ ID NO: 211 - protein, 17 aa, Mus musculus
MNWVKQRPGQGLEWIGQ

SEQ ID NO: 212 - protein, 17 aa, Mus musculus
MSWVRQTPGKTLEWIGD

SEQ ID NO: 213 - protein, 17 aa, Mus musculus
MSWVRQSPEKGLEWVAE

SEQ ID NO: 214 - protein, 17 aa, Mus musculus
MHWVKQRYGQGLEWIGN

SEQ ID NO: 215 - protein, 17 aa, Mus musculus
MHWVKQRPGQGLEWIGM

SEQ ID NO: 216 - protein, 17 aa, Mus musculus
MHWIKQRPEQGLERIGE

SEQ ID NO: 217 - protein, 17 aa, Mus musculus
MHWVKQTPGQGLEWIGV

SEQ ID NO: 218 - protein, 17 aa, Mus musculus
IEWVKQRPGHGLEWIGE

SEQ ID NO: 219 - protein, 17 aa, Mus musculus
MSWVRQPPGKALEWLGF

SEQ ID NO: 220 - protein, 17 aa, Mus musculus
VSWVKQRLGQGLEWIGE

SEQ ID NO: 221 - protein, 17 aa, Mus musculus
MSWVRQTPEKRLEWVAT

SEQ ID NO: 222 - protein, 17 aa, Mus musculus
MNWVRQAPEKGLEWVAR

SEQ ID NO: 223 - protein, 17 aa, Mus musculus
MHWLRQPPGKRLEWIAV

SEQ ID NO: 224 - protein, 7 aa, Mus musculus
ISSGGST

SEQ ID NO: 225 - protein, 8 aa, Mus musculus
INTETGEP

SEQ ID NO: 226 - protein, 8 aa, Mus musculus
INPYNDGT

SEQ ID NO: 227 - protein, 8 aa, Mus musculus
ISSGGGST

SEQ ID NO: 228 - protein, 8 aa, Mus musculus
INPGSGGT

SEQ ID NO: 229 - protein, 8 aa, Mus musculus
IDPSDSET

SEQ ID NO: 230 - protein, 8 aa, Mus musculus
INPSTGYT

SEQ ID NO: 231 - protein, 8 aa, Mus musculus
INPSNGGT

SEQ ID NO: 232 - protein, 8 aa, Mus musculus
INTYTGEP

SEQ ID NO: 233 - protein, 8 aa, Mus musculus
ISNGGGST

SEQ ID NO: 234 - protein, 7 aa, Mus musculus
IWAGGST

SEQ ID NO: 235 - protein, 8 aa, Mus musculus
IDPYDSET

SEQ ID NO: 236 - protein, 8 aa, Mus musculus
INPSSGYT

SEQ ID NO: 237 - protein, 8 aa, Mus musculus
ISSGGSYT

SEQ ID NO: 238 - protein, 7 aa, Mus musculus
IWWNDDK

SEQ ID NO: 239 - protein, 10 aa, Mus musculus
IRSKANNHAT

SEQ ID NO: 240 - protein, 8 aa, Mus musculus
INSNGGST

SEQ ID NO: 241 - protein, 10 aa, Mus musculus
IRLKSNNYAT

SEQ ID NO: 242 - protein, 8 aa, Mus musculus
FNPYSDDI

SEQ ID NO: 243 - protein, 8 aa, Mus musculus
INPYNDYT

SEQ ID NO: 244 - protein, 8 aa, Mus musculus
ISSGGSYI

SEQ ID NO: 245 - protein, 7 aa, Mus musculus
IHYSGST

SEQ ID NO: 246 - protein, 8 aa, Mus musculus
IDPSDSYT

SEQ ID NO: 247 - protein, 8 aa, Mus musculus
IHPGSGGT

SEQ ID NO: 248 - protein, 8 aa, Mus musculus
IYPGDGDT

SEQ ID NO: 249 - protein, 8 aa, Mus musculus
INSDGSAI

SEQ ID NO: 250 - protein, 10 aa, Mus musculus
IRLKSDNYAT

SEQ ID NO: 251 - protein, 8 aa, Mus musculus
IYPGSGST

SEQ ID NO: 252 - protein, 8 aa, Mus musculus
IDPSNSET

SEQ ID NO: 253 - protein, 8 aa, Mus musculus
IYPGNGDT

SEQ ID NO: 254 - protein, 8 aa, Mus musculus
ILPGSGST

SEQ ID NO: 255 - protein, 10 aa, Mus musculus
IRNKANGYTT

SEQ ID NO: 256 - protein, 8 aa, Mus musculus
INPSNSGT

SEQ ID NO: 257 - protein, 10 aa, Mus musculus
IRSKSNNYVT

SEQ ID NO: 258 - protein, 10 aa, Mus musculus
ITVKSDNYGA

SEQ ID NO: 259 - protein, 38 aa, Mus musculus
YYPDSVKGRFTISRDNARNILYLQMSSLRSEDTAMYYC

SEQ ID NO: 260 - protein, 38 aa, Mus musculus
TYADDFKGRFAFSLETSASTAYLQINNLKNEDTATYFC

SEQ ID NO: 261 - protein, 38 aa, Mus musculus
KYNEKFKGKATLTSDKSSSTAYMELSSLTSEDSAVYYC

SEQ ID NO: 262 - protein, 38 aa, Mus musculus
YYPDTVKGRFTISRDNAKNTLYLQMSSLKSEDTAMYYC

SEQ ID NO: 263 - protein, 38 aa, Mus musculus
NYNEKFKGKATLTADKSSSTAYMQLSSLTSDDSAVYFC

SEQ ID NO: 264 - protein, 38 aa, Mus musculus
HYNQMSKDKATLTVDKSSSTAYMQLSSLTSEDSAVYYC

SEQ ID NO: 265 - protein, 38 aa, Mus musculus
EYNQKFKDKATLTADKSSSTAYMQLSSLTSEDSAVYYC

SEQ ID NO: 266 - protein, 38 aa, Mus musculus
NFNEKFKSKATLTVDKSSSTAYMQLSSLTSEDSAVYYC

SEQ ID NO: 267 - protein, 38 aa, Mus musculus
YYPDTVKGRFTISRDNAKNTLYLQMSRLKSEDTAMYYC

SEQ ID NO: 268 - protein, 38 aa, Mus musculus
NYNSALMSRLSISKDNSKSQVFLKMNSLQTDDTAMYYC

SEQ ID NO: 269 - protein, 38 aa, Mus musculus
HYNQKFKDKAILTVDKSSSTAYMQLSSLTSEDSAVYYC

SEQ ID NO: 270 - protein, 38 aa, Mus musculus
NYNQKFKDKATLTADKSSSTAYMQLSSLTSEDSAVYYC

SEQ ID NO: 271 - protein, 38 aa, Mus musculus
YYPDTVTGRFTISRDNAKNTLYLEMSSLRSEDTAMYYC

SEQ ID NO: 272 - protein, 38 aa, Mus musculus
YYNPALKSRLTISKDTSNNQVFLKIASVVTADTATYYC

SEQ ID NO: 273 - protein, 38 aa, Mus musculus
YYAESVKGRFTISRDDSKSSVYLQMNSLRAEDTGIYYC

SEQ ID NO: 274 - protein, 38 aa, Mus musculus
YYPDSVKGRFTISRDNAKNTLYLQMSSLKSEDTAMYYC

SEQ ID NO: 275 - protein, 38 aa, Mus musculus
HYAESVKGRFTISRDDSKSSVYLQMNNLRAEDTGIYYC

SEQ ID NO: 276 - protein, 38 aa, Mus musculus
HYNQKFKDKATLTVDKSSSTAYIQLSSLTSEDSAVYYC

SEQ ID NO: 277 - protein, 38 aa, Mus musculus
SYNQKFKGKATLTVDKSSSTAYMELSSLTSEDSAVYYC

SEQ ID NO: 278 - protein, 38 aa, Mus musculus
YYVDSVKGRFTISRDNAKNTLYLQMSSLKSEDTAIYYC

SEQ ID NO: 279 - protein, 38 aa, Mus musculus
YYAESVKGRFTISRDDSQSSVYLQMNSLRTENTGIYYC

SEQ ID NO: 280 - protein, 38 aa, Mus musculus
NYNSALMSRLGISKDNSKSQVFLKMNSLQTDESAFYYC

SEQ ID NO: 281 - protein, 38 aa, Mus musculus
NYNPSLKSRISITRDTSKNQFFLQLNSVTTEGTATYYC

SEQ ID NO: 282 - protein, 38 aa, Mus musculus
NYNQKFKGKATLAVDKSSSTAYMQLSSLTSEDSAVYYC

SEQ ID NO: 283 - protein, 38 aa, Mus musculus
AYNQKFKGKATLTADKSSSTAYMELSSLTSEDSAVYYC

SEQ ID NO: 284 - protein, 38 aa, Mus musculus
YYNGKFKDKATLTADKSSSTAYMHLSSLTSEDSAVYFC

SEQ ID NO: 285 - protein, 38 aa, Mus musculus
NYAPSIKDRFTIFRDNDKSTLYLQMSNVRSEDTATYFC

SEQ ID NO: 286 - protein, 38 aa, Mus musculus
HYAESVKGKFTISRDDSKSRLYLQMNSLRAEDTGIYYC

SEQ ID NO: 287 - protein, 38 aa, Mus musculus
NYDEKFKSKGTLTVDTSSSTAYMHLSSLTSEDSAVYYC

SEQ ID NO: 288 - protein, 38 aa, Mus musculus
RLNQKFKDKATLNVDKSSNTACMQLSSLTSEDSAVYYC

SEQ ID NO: 289 - protein, 38 aa, Mus musculus
NYNEKFKSKATLTVDKSSSTAYMQLSSLTSEDSAVYYC

SEQ ID NO: 290 - protein, 38 aa, Mus musculus
AYNQKFKGKATVTADRSSSTAYMQLSSLTSEDSAVYYC

SEQ ID NO: 291 - protein, 38 aa, Mus musculus
NYNEKFKGKATFTADTSSNTAYMQLSSLTSEDSAVYYC

SEQ ID NO: 292 - protein, 38 aa, Mus musculus
EYSASVKGRFTISRDNSQSILYLQMNTLRAEDSATYYC

SEQ ID NO: 293 - protein, 38 aa, Mus musculus
YYNEKFKDKVTLTADTSSNTVHIQLSSLTSEDSAVYFC

SEQ ID NO: 294 - protein, 38 aa, Mus musculus
YYPDSVKGRFTISRDNAKNTLYLQMSSLRSEDTAMYYC

SEQ ID NO: 295 - protein, 38 aa, Mus musculus
YYADSLKDRFTISRDDSQSMLYLQMNNLKTEDTCHVLL

SEQ ID NO: 296 - protein, 38 aa, Mus musculus
NYAESVKGRFTISRDDSKSSVYLQMNRLREEDTATYYC

SEQ ID NO: 297 - protein, 13 aa, Mus musculus
ARGEIWGKAWFAY

SEQ ID NO: 298 - protein, 6 aa, Mus musculus
AWGNHY

SEQ ID NO: 299 - protein, 11 aa, Mus musculus
ARDGYYVGPAY

SEQ ID NO: 300 - protein, 10 aa, Mus musculus
ARNYRSWFAY

SEQ ID NO: 301 - protein, 12 aa, Mus musculus
ARKGSLTGVLAY

SEQ ID NO: 302 - protein, 11 aa, Mus musculus
ARRGLRAWFAY

SEQ ID NO: 303 - protein, 14 aa, Mus musculus
ARLDYYGSSRGFAY

SEQ ID NO: 304 - protein, 7 aa, Mus musculus
TNGGGWY

SEQ ID NO: 305 - protein, 13 aa, Mus musculus
ASYYDSTYVGFAY

SEQ ID NO: 306 - protein, 11 aa, Mus musculus
ASPLGYDGFAY

SEQ ID NO: 307 - protein, 8 aa, Mus musculus
ARGAYFDY

SEQ ID NO: 308 - protein, 14 aa, Mus musculus
ARSPAYYGNLWFAY

SEQ ID NO: 309 - protein, 7 aa, Mus musculus
ARWDGAY

SEQ ID NO: 310 - protein, 11 aa, Mus musculus
ARGGMITPFAY

SEQ ID NO: 311 - protein, 15 aa, Mus musculus
ARIGGNDGYYWYFDV

SEQ ID NO: 312 - protein, 6 aa, Mus musculus
TPQFAY

SEQ ID NO: 313 - protein, 8 aa, Mus musculus
ASHYDEGY

SEQ ID NO: 314 - protein, 5 aa, Mus musculus
TGSDY

SEQ ID NO: 315 - protein, 13 aa, Mus musculus
GSGYDGYYDWFAC

SEQ ID NO: 316 - protein, 9 aa, Mus musculus
AREPKTLDY

SEQ ID NO: 317 - protein, 8 aa, Mus musculus
ANWAWFAY

SEQ ID NO: 318 - protein, 11 aa, Mus musculus
ARPYDYDGFAY

SEQ ID NO: 319 - protein, 9 aa, Mus musculus
ADGDYYFDY

SEQ ID NO: 320 - protein, 13 aa, Mus musculus
ARQDDGYYRIFDY

SEQ ID NO: 321 - protein, 10 aa, Mus musculus
TNYGSNPLDY

SEQ ID NO: 322 - protein, 11 aa, Mus musculus
AREGTGPWFAY

SEQ ID NO: 323 - protein, 8 aa, Mus musculus
ARDPPFAY

SEQ ID NO: 324 - protein, 11 aa, Mus musculus
AREEITAWFAY

SEQ ID NO: 325 - protein, 12 aa, Mus musculus
TRNGNGNWYFDV

SEQ ID NO: 326 - protein, 13 aa, Mus musculus
ARSGYRYDAVFAY

SEQ ID NO: 327 - protein, 7 aa, Mus musculus
ARGGNPY

SEQ ID NO: 328 - protein, 11 aa, Mus musculus
MRYGSSYWYFD

SEQ ID NO: 329 - protein, 8 aa, Mus musculus
TRYYYGES

SEQ ID NO: 330 - protein, 14 aa, Mus musculus
ARHYYDYDYWYFDV

SEQ ID NO: 331 - protein, 15 aa, Mus musculus
TRSGVEGLLHWYFDV

SEQ ID NO: 332 - protein, 12 aa, Mus musculus
TCDYDGGAWFAY

SEQ ID NO: 333 - protein, 12 aa, Mus musculus
ARCDGYYDGLDY

SEQ ID NO: 334 - protein, 11 aa, Mus musculus
AREGKNWYFDV

SEQ ID NO: 335 - protein, 10 aa, Mus musculus
ARRIYRTLDY

SEQ ID NO: 336 - protein, 11 aa, Mus musculus
TRSGGNLWFAY

SEQ ID NO: 337 - protein, 13 aa, Mus musculus
ARRTYYGNAWFAY

SEQ ID NO: 338 - protein, 15 aa, Mus musculus
ARDKRITTVEAWFAY

SEQ ID NO: 339 - protein, 10 aa, Mus musculus
ARGYGSSFAY

SEQ ID NO: 340 - protein, 13 aa, Mus musculus
TRGGDYDASWFAY

SEQ ID NO: 341 - protein, 10 aa, Mus musculus
ARRTARAFDY

SEQ ID NO: 342 - protein, 15 aa, Mus musculus
ARRIGYDGGGSWFAY

SEQ ID NO: 343 - protein, 8 aa, Mus musculus
CERVRRCV

SEQ ID NO: 344 - protein, 6 aa, Mus musculus
SRWFAY

SEQ ID NO: 345 - protein, 15 aa, Mus musculus
CARGEIWGKAWFAYW

SEQ ID NO: 346 - protein, 8 aa, Mus musculus
CAWGNHYW

SEQ ID NO: 347 - protein, 13 aa, Mus musculus
CARDGYYVGPAYW

SEQ ID NO: 348 - protein, 12 aa, Mus musculus
CARNYRSWFAYW

SEQ ID NO: 349 - protein, 14 aa, Mus musculus
CARKGSLTGVLAYW

SEQ ID NO: 350 - protein, 13 aa, Mus musculus
CARRGLRAWFAYW

SEQ ID NO: 351 - protein, 16 aa, Mus musculus
CARLDYYGSSRGFAYW

SEQ ID NO: 352 - protein, 9 aa, Mus musculus
CTNGGGWYW

SEQ ID NO: 353 - protein, 15 aa, Mus musculus
CASYYDSTYVGFAYW

SEQ ID NO: 354 - protein, 13 aa, Mus musculus
CASPLGYDGFAYW

SEQ ID NO: 355 - protein, 10 aa, Mus musculus [sequence omitted]

SEQ ID NO: 356 - protein, 16 aa, Mus musculus
CARSPAYYGNLWFAYW

SEQ ID NO: 357 - protein, 9 aa, Mus musculus
CARWDGAYW

SEQ ID NO: 358 - protein, 13 aa, Mus musculus
CARGGMITPFAYW

SEQ ID NO: 359 - protein, 17 aa, Mus musculus
CARIGGNDGYYWYFDVW

SEQ ID NO: 360 - protein, 8 aa, Mus musculus
CTPQFAYW

SEQ ID NO: 361 - protein, 10 aa, Mus musculus
CASHYDEGYW

SEQ ID NO: 362 - protein, 7 aa, Mus musculus
CTGSDYW

SEQ ID NO: 363 - protein, 15 aa, Mus musculus
CGSGYDGYYDWFACW

SEQ ID NO: 364 - protein, 11 aa, Mus musculus
CAREPKTLDYW

SEQ ID NO: 365 - protein, 10 aa, Mus musculus
CANWAWFAYW

SEQ ID NO: 366 - protein, 13 aa, Mus musculus
CARPYDYDGFAYW

SEQ ID NO: 367 - protein, 11 aa, Mus musculus
CADGDYYFDYW

SEQ ID NO: 368 - protein, 15 aa, Mus musculus
CARQDDGYYRIFDYW

SEQ ID NO: 369 - protein, 12 aa, Mus musculus
CTNYGSNPLDYW

SEQ ID NO: 370 - protein, 13 aa, Mus musculus
CAREGTGPWFAYW

SEQ ID NO: 371 - protein, 10 aa, Mus musculus
CARDPPFAYW

SEQ ID NO: 372 - protein, 13 aa, Mus musculus
CAREEITAWFAYW

SEQ ID NO: 373 - protein, 14 aa, Mus musculus
CTRNGNGNWYFDVW

SEQ ID NO: 374 - protein, 15 aa, Mus musculus
CARSGYRYDAVFAYW

SEQ ID NO: 375 - protein, 9 aa, Mus musculus
CARGGNPYL

SEQ ID NO: 376 - protein, 10 aa, Mus musculus
CTRYYYGESW

SEQ ID NO: 377 - protein, 16 aa, Mus musculus
CARHYYDYDYWYFDVW

SEQ ID NO: 378 - protein, 17 aa, Mus musculus
CTRSGVEGLLHWYFDVW

SEQ ID NO: 379 - protein, 14 aa, Mus musculus
CTCDYDGGAWFAYW

SEQ ID NO: 380 - protein, 14 aa, Mus musculus
CARCDGYYDGLDYW

SEQ ID NO: 381 - protein, 13 aa, Mus musculus
CAREGKNWYFDVW

SEQ ID NO: 382 - protein, 12 aa, Mus musculus
CARRIYRTLDYW

SEQ ID NO: 383 - protein, 13 aa, Mus musculus
CTRSGGNLWFAYW

SEQ ID NO: 384 - protein, 15 aa, Mus musculus
CARRTYYGNAWFAYW

SEQ ID NO: 385 - protein, 17 aa, Mus musculus
CARDKRITTVEAWFAYW

SEQ ID NO: 386 - protein, 12 aa, Mus musculus
CARGYGSSFAYW

SEQ ID NO: 387 - protein, 15 aa, Mus musculus
CTRGGDYDASWFAYW

SEQ ID NO: 388 - protein, 12 aa, Mus musculus
CARRTARAFDYW

SEQ ID NO: 389 - protein, 17 aa, Mus musculus
CARRIGYDGGGSWFAYW

SEQ ID NO: 390 - protein, 10 aa, Mus musculus
LCERVRRCVL

SEQ ID NO: 391 - protein, 8 aa, Mus musculus
CSRWFAYW

SEQ ID NO: 392 - protein, 15 aa, Mus musculus
WFAYWGQGTLVTVSA

SEQ ID NO: 393 - protein, 12 aa, Mus musculus
YWGQGTTVTVSS

SEQ ID NO: 394 - protein, 13 aa, Mus musculus
AYWGQGTLVTVSA

SEQ ID NO: 395 - protein, 14 aa, Mus musculus
FAYWGQGTLVTVSA

SEQ ID NO: 396 - protein, 15 aa, Mus musculus
GGWYWGQGTTVTVSS

SEQ ID NO: 397 - protein, 15 aa, Mus musculus
YFDYWGQGTTVTVSS

SEQ ID NO: 398 - protein, 11 aa, Mus musculus
WGQGTPVTVSS

SEQ ID NO: 399 - protein, 14 aa, Mus musculus
FAYWGQGTLVTVSS

SEQ ID NO: 400 - protein, 17 aa, Mus musculus
YWYFDVWGAGTTVTVSS

SEQ ID NO: 401 - protein, 14 aa, Mus musculus
FAYWGQGTTVTVSS

SEQ ID NO: 402 - protein, 12 aa, Mus musculus
YWGQGTSLTVSS

SEQ ID NO: 403 - protein, 13 aa, Mus musculus
DYWGQGTTVTVSS

SEQ ID NO: 404 - protein, 15 aa, Mus musculus
WFACWGQGTLVTVSA

SEQ ID NO: 405 - protein, 15 aa, Mus musculus
YFDYWGQGTSLTVSS

SEQ ID NO: 406 - protein, 14 aa, Mus musculus
FDYWGQGTTLTVSS

SEQ ID NO: 407 - protein, 13 aa, Mus musculus
DYWGQGTTLTIPS

SEQ ID NO: 408 - protein, 14 aa, Mus musculus
WFAYWGQGTTAPSP

SEQ ID NO: 409 - protein, 16 aa, Mus musculus
WYFDVWGAGTTLTVSS

SEQ ID NO: 410 - protein, 14 aa, Mus musculus
FAYWGPGTLVTVSA

SEQ ID NO: 411 - protein, 12 aa, Mus musculus
YLGQGTLVTVSA

SEQ ID NO: 412 - protein, 6 aa, Mus musculus
LLVLRC

SEQ ID NO: 413 - protein, 11 aa, Mus musculus
WGQGTLVTVSS

SEQ ID NO: 414 - protein, 16 aa, Mus musculus
WYFDVWGAGTSLTVSS

SEQ ID NO: 415 - protein, 15 aa, Mus musculus
GLDYWGQGTTLTVSS

SEQ ID NO: 416 - protein, 16 aa, Mus musculus
WYFDVWGAGTTVTVSS

SEQ ID NO: 417 - protein, 15 aa, Mus musculus
TLDYWGQGTTVTVSS

SEQ ID NO: 418 - protein, 15 aa, Mus musculus
SFAYWGQGTPVTVSS

SEQ ID NO: 419 - protein, 14 aa, Mus musculus
FDYWGQGTTVTVSS

SEQ ID NO: 420 - protein, 13 aa, Mus musculus
CVLGPRDSGHCLC

SEQ ID NO: 421 - protein, 11 aa, Mus musculus
WGQGTLVTVSA

SEQ ID NO: 422 - protein, 11 aa, Mus musculus
WGQGTTVTVSS

SEQ ID NO: 423 - protein, 11 aa, Mus musculus
WGAGTTVTVSS

SEQ ID NO: 424 - protein, 11 aa, Mus musculus
WGQGTSLTVSS

SEQ ID NO: 425 - protein, 11 aa, Mus musculus
WGQGTTLTVSS

SEQ ID NO: 426 - protein, 11 aa, Mus musculus
WGQGTTLTIPS

SEQ ID NO: 427 - protein, 10 aa, Mus musculus
WGQGTTAPSP

SEQ ID NO: 428 - protein, 11 aa, Mus musculus
WGAGTTLTVSS

SEQ ID NO: 429 - protein, 11 aa, Mus musculus
WGPGTLVTVSA

SEQ ID NO: 430 - protein, 11 aa, Mus musculus
LGQGTLVTVSA

SEQ ID NO: 431 - protein, 11 aa, Mus musculus
WGAGTSLTVSS

SEQ ID NO: 432 - protein, 120 aa, Mus musculus
QVQLQQSGAELVRPGASVKLSCKALGYTFTDYEMHWVKQTPVHGLGWIGAIHPGSGGTAYNQKFKGKATLTADKSSSTAYMELSSLTSEDSAVYYCTRSDYGSSYEFAYWGQGTLVTVSS

SEQ ID NO: 433 - protein, 117 aa, Mus musculus
QIQLKESGPGLVKPSQSLSLTCTVTGYSITSDYAWNWIRQFPGNKLEWMGYISYSGSTSYNPSLKSRISITRDTSKNQFFLQLNSVTTEDTATYYCARSRGNYFDYWGQGTTVTVSS

SEQ ID NO: 434 - protein, 119 aa, Mus musculus
QIQLQQSGAELARPGASVRMSCKASGYTFTSYTIHWVKQRPGQGLEWIGYINPSSGYTNYNQKFKDKATLTADKSSSTAYMQLSSLTSEDSAVYYCARSGLRQAWFAYWGQGTLVTVSA

SEQ ID NO: 435 - protein, 115 aa, Mus musculus
QVQLVETGGGLVRPGNSLKLSCVTSGFTFSNYRMHWLRQPLGKRLEWIAVITVKSDNYGANYAESVKGRFTISRDDSKSSVYLQMNRLREEDTATYYCSRLFAYWGQGTLVTVSA

SEQ ID NO: 436 - protein, 114 aa, Mus musculus
QVQLQQPGAELVKPGASVKLSCKASGYTFTSYWMHWVKLRPGQGFEWIGEINPSNGGTNYNEKFKRKATLTVDKSSSTAYMQLSSLTSEDSAVYYCTITGFDVWGAGTTVTVSS

SEQ ID NO: 437 - protein, 120 aa, Mus musculus
QIQLQQSGAELVKPGASVKLSCKTSGYTFTTYWIQWVKQRPGQGLGWIGEIFPGTGTTYYNEKFKGKATLTIDTSSSTAYMQLSSLTSEDSAVYFCARGGYYNSSPFAYWGQGTLVTVSA

SEQ ID NO: 438 - protein, 121 aa, Mus musculus
DVQLVESGGGLVQPGGSLRLSCATSGFTFTDYYMSWVRQPPGKALEWLGFIRNKANGYTTEYSASVKGRFTISRDNSQSILYLQMNTLRAEDSATYYCARDGEVRRALAYWGQGTLVTVSA

SEQ ID NO: 439 - protein, 115 aa, Mus musculus
QVQLQQPAAELARPGASVKMSCKASGYTFTSSTMHWVKQRPGQGLEWIGYINPSSGYTEYNQKFKDKTTLTADKSSSTAYMQLSSLTSEDSAVYYCVRHYYFDYWGQGTTVTVSS

SEQ ID NO: 440 - protein, 117 aa, Mus musculus
EVMLVESGGGLVKPGGSLKISCAASGFTFSSYTMSWVRQNPEKRLEWVATISSGGGYTYYLDSVKGRFTPSRDNGKNTLNLQMSSLKSEDTAMYYCTRVSAKYFDVWGAGTTLTVSS

SEQ ID NO: 441 - protein, 120 aa, Mus musculus
QVQLQQSGAELVKPGASVKLSCKASGYTFTNFYLYWVKQRPGQGLEWIGGINPSNGGTNFNEKFKSKATLTVDKSSSTAYMQLSSLTSEDSAVYYCTRSYYDYDWYFDVWGAGTTVTVSS

SEQ ID NO: 442 - protein, 122 aa, Mus musculus
QVQLQQSGAELVRPGASVKLSCKASGYTFTSYWINWVKQRPGQGLEWIGNIYPSDSYTNYNQKFKDKATLTVDKSSSTAYMQLSSPTSEDSAVYYCTRQNYYGSSHWYFDVWGAGTTLTVSS

SEQ ID NO: 443 - protein, 115 aa, Mus musculus
QVQLQQPGAELVKPGAPVKLSCKASGYTFTSYWMNWVKQRPGRGLEWIGRIDPSDSETHYNQKFKDKATLTVDKSSSTAYIQLSSLTSEDSAVYYCANWAWFAYWGQGTLVTVSS

SEQ ID NO: 444 - protein, 121 aa, Mus musculus
QIQFAQSGPELKKPGETVKISCKAFGYTFTDYSMHWVKQAPGKGLKWMGWINTETGEPTYADDFKGRFAFSLETSASTAYLQINNLKNEDTATYFCASFYYGNFAYYFDYRGQGTTLTVSS

SEQ ID NO: 445 - protein, 125 aa, Mus musculus
EVKIEESGGGLVQPGGAMKLSCVASGFTFSNYWMNWVRQSPEKGLEWVAEIRLKSNNYATYYAESVKGRFTISRDDSQSSVYLQMNDLRTEDTGIYYCTRIYDSGSSYTWYFDVWGAGTTVTVSS

SEQ ID NO: 446 - protein, 123 aa, Mus musculus
QVILKESGPGILQPSQTLSLTCSFSGFSLSTSGMSVGWIRQPSGKGLEWLAHIWWNDDKYYNPALKSRLTISKDTPNNQVFLKIASVVTADTATYYCARIGGNDGYYWYFDVWGAGTSLTVSS

SEQ ID NO: 447 - protein, 117 aa, Mus musculus
QIQLQQSGAELVKPGASVKLSCKASGYTFTSYWMHWVKQRPGQGLEWIGEINPSNGRTNYNEKFKSKATLTVDKSSSTAYMQLSSLTSEDSAVYYCARDSSGYGAYWGQGTLVTVSS

SEQ ID NO: 448 - protein, 118 aa, Mus musculus
QVQLQQSGAELAKPGASVKMSCKASGYTFTSYWMHWVKQRPGQGLEWIGYINPSTGYTEYNQKFKDKATLTADKSSSTAYMQLSSLTSEDSAVYYCARYDGYYYFDYWGQGTTLTVSS

SEQ ID NO: 449 - protein, 118 aa, Mus musculus
QVQLQQSGAELVRPGASVKLSCKASGYTFTSYWINWVKQRPGQGLEWIGNIYPSDSYTNYNQKFKDKATLTVDKSSSTAYMQLSSPTSEDSAVYYCTSHYYGRAWFAYWGQGTLVTVL

SEQ ID NO: 450 - protein, 118 aa, Mus musculus
QIQLQQPGAELVKPGASVKLSCKASGYTFTRYYMYWVKQRPGQGLEWIGEINPSNGGTNFNEKFKSKATLTVDKSSSTAYMQLNSLTSDDSAVYYCTKGGFYDFFAYWGQGTLVTVSA

SEQ ID NO: 451 - protein, 112 aa, Mus musculus
EVMLVESGGGLVQPGGSLKLSCAASGFTFSSYGMSWVRQTPDKRLELVATINSNGGSTYYPDSVKGRFTISRDNAKNTLYLQMSSLKSEDTAMYYCASLAYWGQGTLVTVSA

SEQ ID NO: 452 - protein, 116 aa, Mus musculus
QIQLQQSGPDLVKPGASVKISCKASGYTFTDYYINWMKQKPGQGLEWIGWIYPGSGNTKYNEKFKGKATLTVDTSSSTAYMQLSSLTSEDTAVYFCARVYSGFDVWGAGTTVTVSS

SEQ ID NO: 453 - protein, 119 aa, Mus musculus
QIQLQQSGPELVKPGASVKMSCKAPGYTFTSYYIHWVKQRPGQGLEWIGWIYPGDGSTKYNEKFKGKTTLTADKSSSTAYMLLSSLTSEDSAIYFCARGDGYFAWFAYWGQGTLVTVSA

SEQ ID NO: 454 - protein, 119 aa, Mus musculus
QIQLQQSGAELMKPGASVKISCKATGYTFSSYWIEWVKQRPGHGLEWIGEILPGSGSTKYNEKFKGKATFTADTSSNTAYMQLSSLTSEDSAVYYCARSAHRYDAWFAYWGQGTLVTVL

SEQ ID NO: 455 - protein, 123 aa, Mus musculus
QIQLQQPGAELVKPGASVKLSCKASGYTFTNYYMYWVNQRPGQGLEWIGGINPTNGGTNFNAKFKNKATLTVDKSSNTAYMQLSSLTSEDSAVYYCTRGMAYRYDGAGWFAYWGQGTPVTVSS

SEQ ID NO: 456 - protein, 118 aa, Mus musculus
QVQLQQPGAELVRPGASVKLSCKASGYTFTSYWMNWVKQRPEQGLEWIGRIDPYDSETHYNQKFKDKAILTVDKSSSTAYMQLSSLTSEDSAVYYCARGGRGTWFAYWGQGTLVTVSA

SEQ ID NO: 457 - protein, 119 aa, Mus musculus
DVMLVESGGGLVQPGGSLRLSCATSGFTFTDYYMSWVRQPPGKALEWLGFIRNKANGYTTEYSASVKGRFTISRDNSQSILYLQMNTLRAEDSATYYCARGWGNWFAYWGQGTLVTVSA

SEQ ID NO: 458 - protein, 117 aa, Mus musculus
EVMLVESGGGLVQPGGSRKLSCAASGFTFSSFGIHWVRQAPEKGLEWVAYISGGGGTISYADTVKGRFTISRDNPKNTLFLQMTSLRSEDTAIYYCARWRGGYFDYWGQGTSLTVSS

SEQ ID NO: 459 - protein, 121 aa, Mus musculus
QIQLQQSGAELVRPGASVKLSCKASGYTFTSYWINWVKQRPGQGLEWIGNIYPSDSYTNYNQKFKDKATLTVDKSSSTAYMQLSSPTSEDSAVYYCTRTGGSTMTPWFAYWGQGTLVTVSS

SEQ ID NO: 460 - protein, 121 aa, Mus musculus
QVTLKESGPGILQPSQTLSLTCSFSGFSLSTSGMGVSWIRQPSGKGLEWLAHIYWDDDKRYNPSLKSRLTISKDTSRNQVFLKITSVDTTDTATYYCARRAGDYGNPFPYWGQGTLVTVSA

SEQ ID NO: 461 - protein, 25 aa, Mus musculus
QVQLQQSGAELVRPGASVKLSCKAL

SEQ ID NO: 462 - protein, 25 aa, Mus musculus
QIQLKESGPGLVKPSQSLSLTCTVT

SEQ ID NO: 463 - protein, 25 aa, Mus musculus
QIQLQQSGAELARPGASVRMSCKAS

SEQ ID NO: 464 - protein, 25 aa, Mus musculus
QVQLQQPGAELVKPGASVKLSCKAS

SEQ ID NO: 465 - protein, 25 aa, Mus musculus
QIQLQQSGAELVKPGASVKLSCKTS

SEQ ID NO: 466 - protein, 25 aa, Mus musculus
DVQLVESGGGLVQPGGSLRLSCATS

SEQ ID NO: 467 - protein, 25 aa, Mus musculus
QVQLQQPAAELARPGASVKMSCKAS

SEQ ID NO: 468 - protein, 25 aa, Mus musculus
EVMLVESGGGLVKPGGSLKISCAAS

SEQ ID NO: 469 - protein, 25 aa, Mus musculus
QVQLQQPGAELVKPGAPVKLSCKAS

SEQ ID NO: 470 - protein, 25 aa, Mus musculus
QIQFAQSGPELKKPGETVKISCKAF

SEQ ID NO: 471 - protein, 25 aa, Mus musculus
EVKIEESGGGLVQPGGAMKLSCVAS

SEQ ID NO: 472 - protein, 25 aa, Mus musculus
QIQLQQSGAELVKPGASVKLSCKAS

SEQ ID NO: 473 - protein, 25 aa, Mus musculus
QVQLQQSGAELAKPGASVKMSCKAS

SEQ ID NO: 474 - protein, 25 aa, Mus musculus
QIQLQQPGAELVKPGASVKLSCKAS

SEQ ID NO: 475 - protein, 25 aa, Mus musculus
QIQLQQSGPDLVKPGASVKISCKAS

SEQ ID NO: 476 - protein, 25 aa, Mus musculus
QIQLQQSGPELVKPGASVKMSCKAP

SEQ ID NO: 477 - protein, 25 aa, Mus musculus
QVQLQQPGAELVRPGASVKLSCKAS

SEQ ID NO: 478 - protein, 25 aa, Mus musculus
DVMLVESGGGLVQPGGSLRLSCATS

SEQ ID NO: 479 - protein, 25 aa, Mus musculus
EVMLVESGGGLVQPGGSRKLSCAAS

SEQ ID NO: 480 - protein, 25 aa, Mus musculus
QIQLQQSGAELVRPGASVKLSCKAS

SEQ ID NO: 481 - protein, 25 aa, Mus musculus
QVTLKESGPGILQPSQTLSLTCSFS

SEQ ID NO: 482 - protein, 9 aa, Mus musculus
GYSITSDYA

SEQ ID NO: 483 - protein, 8 aa, Mus musculus
GYTFTTYW

SEQ ID NO: 484 - protein, 8 aa, Mus musculus
GYTFTSST

SEQ ID NO: 485 - protein, 8 aa, Mus musculus
GFTFSSYT

SEQ ID NO: 486 - protein, 8 aa, Mus musculus
GYTFTNFY

SEQ ID NO: 487 - protein, 8 aa, Mus musculus
GYTFTDYS

SEQ ID NO: 488 - protein, 8 aa, Mus musculus
GYTFTRYY

SEQ ID NO: 489 - protein, 8 aa, Mus musculus
GYTFTDYY

SEQ ID NO: 490 - protein, 8 aa, Mus musculus
GYTFTNYY

SEQ ID NO: 491 - protein, 8 aa, Mus musculus
GFTFSSFG

SEQ ID NO: 492 - protein, 10 aa, Mus musculus
GFSLSTSGMG

SEQ ID NO: 493 - protein, 17 aa, Mus musculus
MHWVKQTPVHGLGWIGA

SEQ ID NO: 494 - protein, 17 aa, Mus musculus
WNWIRQFPGNKLEWMGY

SEQ ID NO: 495 - protein, 17 aa, Mus musculus
MHWLRQPLGKRLEWIAV

SEQ ID NO: 496 - protein, 17 aa, Mus musculus
MHWVKLRPGQGFEWIGE

SEQ ID NO: 497 - protein, 17 aa, Mus musculus
IQWVKQRPGQGLGWIGE

SEQ ID NO: 498 - protein, 17 aa, Mus musculus
MSWVRQNPEKRLEWVAT

SEQ ID NO: 499 - protein, 17 aa, Mus musculus
LYWVKQRPGQGLEWIGG

SEQ ID NO: 500 - protein, 17 aa, Mus musculus
INWVKQRPGQGLEWIGN

SEQ ID NO: 501 - protein, 17 aa, Mus musculus
INWMKQKPGQGLEWIGW

SEQ ID NO: 502 - protein, 17 aa, Mus musculus
IHWVKQRPGQGLEWIGW

SEQ ID NO: 503 - protein, 17 aa, Mus musculus
MYWVNQRPGQGLEWIGG

SEQ ID NO: 504 - protein, 17 aa, Mus musculus
IHWVRQAPEKGLEWVAY

SEQ ID NO: 505 - protein, 17 aa, Mus musculus
VSWIRQPSGKGLEWLAH

SEQ ID NO: 506 - protein, 7 aa, Mus musculus
ISYSGST

SEQ ID NO: 507 - protein, 8 aa, Mus musculus
IFPGTGTT

SEQ ID NO: 508 - protein, 8 aa, Mus musculus
ISSGGGYT

SEQ ID NO: 509 - protein, 8 aa, Mus musculus
IYPSDSYT

SEQ ID NO: 510 - protein, 8 aa, Mus musculus
INPSNGRT

SEQ ID NO: 511 - protein, 8 aa, Mus musculus
IYPGSGNT

SEQ ID NO: 512 - protein, 8 aa, Mus musculus
IYPGDGST

SEQ ID NO: 513 - protein, 8 aa, Mus musculus
INPTNGGT

SEQ ID NO: 514 - protein, 8 aa, Mus musculus
ISGGGGTI

SEQ ID NO: 515 - protein, 7 aa, Mus musculus
IYWDDDK

SEQ ID NO: 516 - protein, 38 aa, Mus musculus
SYNPSLKSRISITRDTSKNQFFLQLNSVTTEDTATYYC

SEQ ID NO: 517 - protein, 38 aa, Mus musculus
NYNEKFKRKATLTVDKSSSTAYMQLSSLTSEDSAVYYC

SEQ ID NO: 518 - protein, 38 aa, Mus musculus
YYNEKFKGKATLTIDTSSSTAYMQLSSLTSEDSAVYFC

SEQ ID NO: 519 - protein, 38 aa, Mus musculus
EYNQKFKDKTTLTADKSSSTAYMQLSSLTSEDSAVYYC

SEQ ID NO: 520 - protein, 38 aa, Mus musculus
YYLDSVKGRFTPSRDNGKNTLNLQMSSLKSEDTAMYYC

SEQ ID NO: 521 - protein, 38 aa, Mus musculus
NYNQKFKDKATLTVDKSSSTAYMQLSSPTSEDSAVYYC

SEQ ID NO: 522 - protein, 38 aa, Mus musculus
YYAESVKGRFTISRDDSQSSVYLQMNDLRTEDTGIYYC

SEQ ID NO: 523 - protein, 38 aa, Mus musculus
YYNPALKSRLTISKDTPNNQVFLKIASVVTADTATYYC

SEQ ID NO: 524 - protein, 38 aa, Mus musculus
NFNEKFKSKATLTVDKSSSTAYMQLNSLTSDDSAVYYC

SEQ ID NO: 525 - protein, 38 aa, Mus musculus
KYNEKFKGKATLTVDTSSSTAYMQLSSLTSEDTAVYFC

SEQ ID NO: 526 - protein, 38 aa, Mus musculus
KYNEKFKGKTTLTADKSSSTAYMLLSSLTSEDSAIYFC

SEQ ID NO: 527 - protein, 38 aa, Mus musculus
KYNEKFKGKATFTADTSSNTAYMQLSSLTSEDSAVYYC

SEQ ID NO: 528 - protein, 38 aa, Mus musculus
NFNAKFKNKATLTVDKSSNTAYMQLSSLTSEDSAVYYC

SEQ ID NO: 529 - protein, 38 aa, Mus musculus
SYADTVKGRFTISRDNPKNTLFLQMTSLRSEDTAIYYC

SEQ ID NO: 530 - protein, 38 aa, Mus musculus
RYNPSLKSRLTISKDTSRNQVFLKITSVDTTDTATYYC

SEQ ID NO: 531 - protein, 13 aa, Mus musculus
TRSDYGSSYEFAY

SEQ ID NO: 532 - protein, 10 aa, Mus musculus
ARSRGNYFDY

SEQ ID NO: 533 - protein, 12 aa, Mus musculus
ARSGLRQAWFAY

SEQ ID NO: 534 - protein, 6 aa, Mus musculus
SRLFAY

SEQ ID NO: 535 - protein, 7 aa, Mus musculus
TITGFDV

SEQ ID NO: 536 - protein, 13 aa, Mus musculus
ARGGYYNSSPFAY

SEQ ID NO: 537 - protein, 12 aa, Mus musculus
ARDGEVRRALAY

SEQ ID NO: 538 - protein, 8 aa, Mus musculus
VRHYYFDY

SEQ ID NO: 539 - protein, 10 aa, Mus musculus
TRVSAKYFDV

SEQ ID NO: 540 - protein, 13 aa, Mus musculus
TRSYYDYDWYFDV

SEQ ID NO: 541 - protein, 15 aa, Mus musculus
TRQNYYGSSHWYFDV

SEQ ID NO: 542 - protein, 14 aa, Mus musculus
ASFYYGNFAYYFDY

SEQ ID NO: 543 - protein, 16 aa, Mus musculus
TRIYDSGSSYTWYFDV

SEQ ID NO: 544 - protein, 10 aa, Mus musculus
ARDSSGYGAY

SEQ ID NO: 545 - protein, 11 aa, Mus musculus
ARYDGYYYFDY

SEQ ID NO: 546 - protein, 12 aa, Mus musculus
TSHYYGRAWFAY

SEQ ID NO: 547 - protein, 11 aa, Mus musculus
TKGGFYDFFAY

SEQ ID NO: 548 - protein, 5 aa, Mus musculus
ASLAY

SEQ ID NO: 549 - protein, 9 aa, Mus musculus
ARVYSGFDV

SEQ ID NO: 550 - protein, 12 aa, Mus musculus
ARGDGYFAWFAY

SEQ ID NO: 551 - protein, 13 aa, Mus musculus
ARSAHRYDAWFAY

SEQ ID NO: 552 - protein, 16 aa, Mus musculus
TRGMAYRYDGAGWFAY

SEQ ID NO: 553 - protein, 11 aa, Mus musculus
ARGGRGTWFAY

SEQ ID NO: 554 - protein, 10 aa, Mus musculus
ARGWGNWFAY

SEQ ID NO: 555 - protein, 10 aa, Mus musculus
ARWRGGYFDY

SEQ ID NO: 556 - protein, 14 aa, Mus musculus
TRTGGSTMTPWFAY

SEQ ID NO: 557 - protein, 13 aa, Mus musculus
ARRAGDYGNPFPY

SEQ ID NO: 558 - protein, 15 aa, Mus musculus
CTRSDYGSSYEFAYW

SEQ ID NO: 559 - protein, 12 aa, Mus musculus
CARSRGNYFDYW

SEQ ID NO: 560 - protein, 14 aa, Mus musculus
CARSGLRQAWFAYW

SEQ ID NO: 561 - protein, 8 aa, Mus musculus
CSRLFAYW

SEQ ID NO: 562 - protein, 9 aa, Mus musculus
CTITGFDVW

SEQ ID NO: 563 - protein, 15 aa, Mus musculus
CARGGYYNSSPFAYW

SEQ ID NO: 564 - protein, 14 aa, Mus musculus
CARDGEVRRALAYW

SEQ ID NO: 565 - protein, 10 aa, Mus musculus
CVRHYYFDYW

SEQ ID NO: 566 - protein, 12 aa, Mus musculus
CTRVSAKYFDVW

SEQ ID NO: 567 - protein, 15 aa, Mus musculus
CTRSYYDYDWYFDVW

SEQ ID NO: 568 - protein, 17 aa, Mus musculus
CTRQNYYGSSHWYFDVW

SEQ ID NO: 569 - protein, 16 aa, Mus musculus
CASFYYGNFAYYFDYR

SEQ ID NO: 570 - protein, 18 aa, Mus musculus
CTRIYDSGSSYTWYFDVW

SEQ ID NO: 571 - protein, 12 aa, Mus musculus
CARDSSGYGAYW

SEQ ID NO: 572 - protein, 13 aa, Mus musculus
CARYDGYYYFDYW

SEQ ID NO: 573 - protein, 14 aa, Mus musculus
CTSHYYGRAWFAYW

SEQ ID NO: 574 - protein, 13 aa, Mus musculus
CTKGGFYDFFAYW

SEQ ID NO: 575 - protein, 7 aa, Mus musculus
CASLAYW

SEQ ID NO: 576 - protein, 11 aa, Mus musculus
CARVYSGFDVW

SEQ ID NO: 577 - protein, 14 aa, Mus musculus
CARGDGYFAWFAYW

SEQ ID NO: 578 - protein, 15 aa, Mus musculus
CARSAHRYDAWFAYW

SEQ ID NO: 579 - protein, 18 aa, Mus musculus
CTRGMAYRYDGAGWFAYW

SEQ ID NO: 580 - protein, 13 aa, Mus musculus
CARGGRGTWFAYW

SEQ ID NO: 581 - protein, 12 aa, Mus musculus
CARGWGNWFAYW

SEQ ID NO: 582 - protein, 12 aa, Mus musculus
CARWRGGYFDYW

SEQ ID NO: 583 - protein, 16 aa, Mus musculus
CTRTGGSTMTPWFAYW

SEQ ID NO: 584 - protein, 15 aa, Mus musculus
CARRAGDYGNPFPYW

SEQ ID NO: 585 - protein, 14 aa, Mus musculus
FDVWGAGTTVTVSS

SEQ ID NO: 586 - protein, 15 aa, Mus musculus
YFDVWGAGTTLTVSS

SEQ ID NO: 587 - protein, 17 aa, Mus musculus
DWYFDVWGAGTTVTVSS

SEQ ID NO: 588 - protein, 15 aa, Mus musculus
WFAYWGQGTLVTVSS

SEQ ID NO: 589 - protein, 15 aa, Mus musculus
YFDYRGQGTTLTVSS

SEQ ID NO: 590 - protein, 17 aa, Mus musculus
YWYFDVWGAGTSLTVSS

SEQ ID NO: 591 - protein, 15 aa, Mus musculus
YGAYWGQGTLVTVSS

SEQ ID NO: 592 - protein, 15 aa, Mus musculus
YFDYWGQGTTLTVSS

SEQ ID NO: 593 - protein, 14 aa, Mus musculus
WFAYWGQGTLVTVL

SEQ ID NO: 594 - protein, 15 aa, Mus musculus
FFAYWGQGTLVTVSA

SEQ ID NO: 595 - protein, 15 aa, Mus musculus
WFAYWGQGTPVTVSS

SEQ ID NO: 596 - protein, 14 aa, Mus musculus
FPYWGQGTLVTVSA

SEQ ID NO: 597 - protein, 11 aa, Mus musculus
RGQGTTLTVSS

SEQ ID NO: 598 - protein, 10 aa, Mus musculus
WGQGTLVTVL

SEQ ID NO: 599-600 - skipped (2 entries)

SEQ ID NO: 601 - protein, 14 aa, Mus musculus
AREASYYYGNAWFA

SEQ ID NO: 602 - protein, 12 aa, Mus musculus
ARGGTSFVHFDY

SEQ ID NO: 603 - protein, 14 aa, Mus musculus
ARGGTSVVHFDFDY

SEQ ID NO: 604 - protein, 12 aa, Mus musculus
ARGGTSVVHFDS

SEQ ID NO: 605 - protein, 12 aa, Mus musculus
ARGGTSVVHFDY

SEQ ID NO: 606 - protein, 8 aa, Mus musculus
ARGGYFDY

SEQ ID NO: 607 - protein, 8 aa, Mus musculus
ARGTVVDY

SEQ ID NO: 608 - protein, 9 aa, Mus musculus
ARGTVVVDY

SEQ ID NO: 609 - protein, 8 aa, Mus musculus
ARGVTFDS

SEQ ID NO: 610 - protein, 8 aa, Mus musculus
ARGVTFDY

SEQ ID NO: 611 - protein, 8 aa, Mus musculus
ARHGYFDY

SEQ ID NO: 612 - protein, 8 aa, Mus musculus
ARHYYFDY

SEQ ID NO: 613 - protein, 8 aa, Mus musculus
ARHYYFDY

SEQ ID NO: 614 - protein, 7 aa, Mus musculus
ARQLAAY

SEQ ID NO: 615 - protein, 7 aa, Mus musculus
ARVSPAS

SEQ ID NO: 616 - protein, 7 aa, Mus musculus
ARVTPAS

SEQ ID NO: 617 - protein, 8 aa, Mus musculus
ARWGNFDY

SEQ ID NO: 618 - skipped

SEQ ID NO: 619 - protein, 8 aa, Mus musculus
  VARIANT 2: note = Xaa can be any naturally occurring amino acid
AXGVTFDY

SEQ ID NO: 620 - protein, 11 aa, Mus musculus
CFQGSHVPWTF

SEQ ID NO: 621 - protein, 11 aa, Mus musculus
CFQGSHVPYTF

SEQ ID NO: 622 - protein, 11 aa, Mus musculus
CGQSYRYPYTF

SEQ ID NO: 623 - protein, 11 aa, Mus musculus
CGQSYSYPWTF

SEQ ID NO: 624 - protein, 11 aa, Mus musculus
CGQSYSYPYTF

SEQ ID NO: 625 - protein, 11 aa, Mus musculus
CHQLVEYPYTF

SEQ ID NO: 626 - protein, 11 aa, Mus musculus
CHQRGSYPWTF

SEQ ID NO: 627 - protein, 11 aa, Mus musculus
CHQRSGYSYTF

SEQ ID NO: 628 - protein, 11 aa, Mus musculus
CHQYHRSPPTF

SEQ ID NO: 629 - protein, 12 aa, Mus musculus
CLQGTYYPTWTF

SEQ ID NO: 630 - protein, 11 aa, Mus musculus
CLQHWNYPYTF

SEQ ID NO: 631 - protein, 11 aa, Mus musculus
CLQSDNMPLTF

SEQ ID NO: 632 - protein, 11 aa, Mus musculus
CLQSDNMPYTF

SEQ ID NO: 633 - protein, 11 aa, Mus musculus
CLQVTHVPFAF

SEQ ID NO: 634 - protein, 11 aa, Mus musculus
CLQYASSPHTF

SEQ ID NO: 635 - protein, 11 aa, Mus musculus
CLQYASSPRTF

SEQ ID NO: 636 - protein, 11 aa, Mus musculus
CLQYASSPWTF

SEQ ID NO: 637 - protein, 11 aa, Mus musculus
CLQYASSPYTF

SEQ ID NO: 638 - protein, 11 aa, Mus musculus
CLQYASYPRTF

SEQ ID NO: 639 - protein, 11 aa, Mus musculus
CLQYATFPYTF

SEQ ID NO: 640 - protein, 11 aa, Mus musculus [sequence omitted]

SEQ ID NO: 641 - protein, 11 aa, Mus musculus
CMQHLEYPYTF

SEQ ID NO: 642 - protein, 11 aa, Mus musculus
CQHFWGTPPTF

SEQ ID NO: 643 - protein, 11 aa, Mus musculus
CQHFWGTPWTF

SEQ ID NO: 644 - protein, 11 aa, Mus musculus
CQHHYGTPWTF

SEQ ID NO: 645 - protein, 11 aa, Mus musculus
CQNGHSFPRTF

SEQ ID NO: 646 - protein, 11 aa, Mus musculus
CQQDYSSPPTF

SEQ ID NO: 647 - protein, 11 aa, Mus musculus
CQQDYSSPWTF

SEQ ID NO: 648 - protein, 11 aa, Mus musculus
CQQGNTLPRTF

SEQ ID NO: 649 - protein, 11 aa, Mus musculus
CQQGNTLPWTF

SEQ ID NO: 650 - protein, 11 aa, Mus musculus
CQQHFNSPYTF

SEQ ID NO: 651 - protein, 11 aa, Mus musculus
CQQHNEYPWTF

SEQ ID NO: 652 - protein, 11 aa, Mus musculus
CQQHYSTHVHV

SEQ ID NO: 653 - protein, 11 aa, Mus musculus [sequence omitted]

SEQ ID NO: 654 - protein, 12 aa, Mus musculus
CQQHYSTPPWTF

SEQ ID NO: 655 - protein, 11 aa, Mus musculus [sequence omitted]

SEQ ID NO: 656 - protein, 11 aa, Mus musculus [sequence omitted]

SEQ ID NO: 657 - protein, 11 aa, Mus musculus [sequence omitted]

SEQ ID NO: 658 - protein, 11 aa, Mus musculus
CQQNNEDPYTF

SEQ ID NO: 659 - protein, 11 aa, Mus musculus
CQQSKEVPYTF

SEQ ID NO: 660 - protein, 11 aa, Mus musculus
CQQSNEDPLTF

SEQ ID NO: 661 - protein, 11 aa, Mus musculus
CQQSNEDPRTF

SEQ ID NO: 662 - protein, 11 aa, Mus musculus
CQQSNEDPYTF

SEQ ID NO: 663 - protein, 11 aa, Mus musculus
CQQVYTLPWTF

SEQ ID NO: 664 - protein, 11 aa, Mus musculus
CQQWSSNPPTF

SEQ ID NO: 665 - protein, 12 aa, Mus musculus
CQQWSSNPPYTF

SEQ ID NO: 666 - protein, 11 aa, Mus musculus
CQQWSSNPYTF

SEQ ID NO: 667 - protein, 11 aa, Mus musculus
CQQWSSYPYTS

SEQ ID NO: 668 - protein, 11 aa, Mus musculus
CQQWTSNPPTF

SEQ ID NO: 669 - protein, 11 aa, Mus musculus [sequence omitted]

SEQ ID NO: 670 - protein, 11 aa, Mus musculus [sequence omitted]

SEQ ID NO: 671 - protein, 11 aa, Mus musculus [sequence omitted]

SEQ ID NO: 672 - protein, 11 aa, Mus musculus
CQQYSKLPWTF

SEQ ID NO: 673 - protein, 11 aa, Mus musculus [sequence omitted]

SEQ ID NO: 674 - protein, 11 aa, Mus musculus
CQQYWSTPPTF

SEQ ID NO: 675 - protein, 9 aa, Mus musculus [sequence omitted]

SEQ ID NO: 676 - protein, 11 aa, Mus musculus [sequence omitted]

SEQ ID NO: 677 - protein, 10 aa, Mus musculus [sequence omitted]

SEQ ID NO: 678 - protein, 11 aa, Mus musculus
CSQSTHVPHTF

SEQ ID NO: 679 - protein, 11 aa, Mus musculus
CSQSTHVPLTF

SEQ ID NO: 680 - protein, 11 aa, Mus musculus
CSQSTHVPRTF

SEQ ID NO: 681 - protein, 10 aa, Mus musculus
CSQSTHVPTF

SEQ ID NO: 682 - protein, 11 aa, Mus musculus
CSQSTHVPWTF

SEQ ID NO: 683 - protein, 11 aa, Mus musculus
CSQSTHVPYTF

SEQ ID NO: 684 - protein, 10 aa, Mus musculus
CSQSTHVYTF

SEQ ID NO: 685 - protein, 11 aa, Mus musculus
CWQGAHFPWTF

SEQ ID NO: 686 - protein, 10 aa, Mus musculus
CWQGTFSSHV

SEQ ID NO: 687 - protein, 11 aa, Mus musculus
CWQGTHFPQTF

SEQ ID NO: 688 - protein, 11 aa, Mus musculus
CWQGTHFPRTF

SEQ ID NO: 689 - protein, 11 aa, Mus musculus
CWQGTHFPWTF

SEQ ID NO: 690 - protein, 10 aa, Mus musculus
CWQGTHFRTF

SEQ ID NO: 691 - protein, 17 aa, Mus musculus
DFYPGGDYINYNEKFKG

SEQ ID NO: 692 - protein, 17 aa, Mus musculus
DIHPGGDYSNYNEKFKG

SEQ ID NO: 693 - protein, 17 aa, Mus musculus
DIHPGGDYTNYNEKFKG

SEQ ID NO: 694 - protein, 17 aa, Mus musculus
DIHPGGGYIDYNEKFTG

SEQ ID NO: 695 - protein, 17 aa, Mus musculus
DIHPGGGYINYNEKFTG

SEQ ID NO: 696 - protein, 17 aa, Mus musculus
DIHPGGGYTNYNEKFKG

SEQ ID NO: 697 - protein, 17 aa, Mus musculus
DIHPGGSYTNYNENFKG

SEQ ID NO: 698 - protein, 26 aa, Mus musculus
DILLTQSPALMAASPGEKVTITCSVS

SEQ ID NO: 699 - protein, 23 aa, Mus musculus
DILLTQSQKFMSTSVGDRVSVTC

SEQ ID NO: 700 - protein, 26 aa, Mus musculus
DILLTQSQKFMSTSVGDRVSVTCKAS

SEQ ID NO: 701 - protein, 107 aa, Mus musculus
DILLTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 702 - protein, 26 aa, Mus musculus
DILMTQSPAIMSASPGEKVTMTCSAS

SEQ ID NO: 703 - protein, 106 aa, Mus musculus
DILMTQSPAIMSASPGEKVTMTCSASSSVIYMHWYQQKSGTSPKRWIYDTSKLASGVPARFSGSGSGTSYSLSISSMETEDAATYYCQQWTSNPPTFGGGTKLEIK

SEQ ID NO: 704 - protein, 26 aa, Mus musculus
DILMTQSPASLSVATGEKVTIRCITS

SEQ ID NO: 705 - protein, 107 aa, Mus musculus
DILMTQSPASLSVATGEKVTIRCITSTDIDDDMNWYQQKPGEPPKLLISEGNTLRPGVPSRFSSSGYGTDFVFTIENTLSEDVADYYCLQSDNMPLTFGGGTKLEIK

SEQ ID NO: 706 - protein, 26 aa, Mus musculus
DILMTQSPATLSVTPGDRVSLSCRAS

SEQ ID NO: 707 - protein, 107 aa, Mus musculus
DILMTQSPATLSVTPGDRVSLSCRASQSISDYLHWYQQKSHESPRLLIKYASQSISGIPSRFSGSGSGSDFTLSINSVEPEDVGVYYCQNGHSFPRTFGGGTKLEIK

SEQ ID NO: 708 - protein, 26 aa, Mus musculus
DILMTQSPSSLAVSVGEKVTMSCKSS

SEQ ID NO: 709 - protein, 113 aa, Mus musculus
DILMTQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPWTFGGGTKLEMK

SEQ ID NO: 710 - protein, 23 aa, Mus musculus
DILMTQSQKFMSTSVGDRVSVTC

SEQ ID NO: 711 - protein, 26 aa, Mus musculus
DILMTQSQKFMSTSVGDRVSVTCKAS

SEQ ID NO: 712 - protein, 107 aa, Mus musculus
DILMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 713 - protein, 26 aa, Mus musculus
DIQMNQSHKFMSTSVGDRVSITCKAS

SEQ ID NO: 714 - protein, 26 aa, Mus musculus
DIQMNQSPSSLSASLGERVSLTCRAS

SEQ ID NO: 715 - protein, 107 aa, Mus musculus
DIQMNQSPSSLSASLGERVSLTCRASQDIGSSLNWLQQEPDGTIKRLIYATSSLDSGVPKRFSGSRSGSDYSLTISSLESEDFVDYYCLQYASSPYTFGGGTKLEIK

SEQ ID NO: 716 - protein, 26 aa, Mus musculus
DIQMNQSPSYLAASPGETITINCRAS

SEQ ID NO: 717 - protein, 107 aa, Mus musculus
DIQMNQSPSYLAASPGETITINCRASKSISKYLAWYQEKPGKTNKLLIYSGSTLQSGIPSRFSGSGSGTDFTLTISSLEPEDFAMYYCQQHNEYPWTFGGGTKLEIK

SEQ ID NO: 718 - protein, 26 aa, Mus musculus
DIQMTQSPASLSASVGESVTITCRAS

SEQ ID NO: 719 - protein, 26 aa, Mus musculus
DIQMTQSPASLSASVGESVTITCRTS

SEQ ID NO: 720 - protein, 26 aa, Mus musculus
DIQMTQSPASLSASVGETVTITCRAS

SEQ ID NO: 721 - protein, 26 aa, Mus musculus
DIQMTQSPASLSVSVGETVAITCRAS

SEQ ID NO: 722 - protein, 107 aa, Mus musculus
DIQMTQSPASLSVSVGETVAITCRASENIYSNLAWYQQKQGKSPQLLVYAATNLADGVPSRFSGSGSGTQYSLKINSLQSEDFGSYYCQHFWGTPWTFGGGTKLEMK

SEQ ID NO: 723 - protein, 26 aa, Mus musculus
DIQMTQSPASLSVSVGETVTITCRAS

SEQ ID NO: 724 - protein, 107 aa, Mus musculus
DIQMTQSPASLSVSVGETVTITCRASENIYSNLAWYQQKQGKSPQLLVYAATNLADGVPSRFSGSGSGTQYSLKINSLQSEDFGSYYCQHFWGTPPTFGGGTKLEIK

SEQ ID NO: 725 - protein, 107 aa, Mus musculus
DIQMTQSPASLSVSVGETVTITCRASENIYSNLAWYQQKQGKSPQLLVYAATNLADGVPSRFSGSGSGTQYSLKINSLQSEDFGSYYCQHFWGTPWTFGGGTKLEMK

SEQ ID NO: 726 - protein, 26 aa, Mus musculus
DIQMTQSPSSLSASLGERVSLTCRAS

SEQ ID NO: 727 - protein, 107 aa, Mus musculus
DIQMTQSPSSLSASLGERVSLTCRASQDIGSSLNWLQQEPDGTIKRLIYATSSLDSGVPKRFSGSRSGSDYSLTISSLESEDFVDYYCLQYASSPHTFGGGTKLEIK

SEQ ID NO: 728 - protein, 107 aa, Mus musculus
DIQMTQSPSSLSASLGERVSLTCRASQEISGYLSWLQQKPDGTIKRLIYAASTLDSGVPKRFSGSRSGSDYSLTISSLESEDFADYYCLQYASYPRTFGGGTKLEIK

SEQ ID NO: 729 - protein, 26 aa, Mus musculus
DIQMTQSQKFMSASVGDRVSVTCKAS

SEQ ID NO: 730 - protein, 26 aa, Mus musculus
DIQMTQSQKFMSTSAGDRVSVTCKAS

SEQ ID NO: 731 - protein, 26 aa, Mus musculus
DIQMTQSQKFMSTSVGDRASVTCKAS

SEQ ID NO: 732 - protein, 26 aa, Mus musculus
DIQMTQSQKFMSTSVGDRVGVTCKAS

SEQ ID NO: 733 - protein, 23 aa, Mus musculus
DIQMTQSQKFMSTSVGDRVSVTC

SEQ ID NO: 734 - protein, 26 aa, Mus musculus
DIQMTQSQKFMSTSVGDRVSVTCKAS

SEQ ID NO: 735 - protein, 107 aa, Mus musculus
DIQMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEMK

SEQ ID NO: 736 - protein, 107 aa, Mus musculus
DIQMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 737 - protein, 107 aa, Mus musculus
DIQMTQSQKFMSTSVGDRVSVTCKASQSVGTNVAWYQQKPGQSPKALIYSASYRYSGVPDRFTGSGSGTDFTLTISNVQSEDLAEYFCQQYNNYPWTFGGGTKLEIK

SEQ ID NO: 738 - protein, 26 aa, Mus musculus
DIQMTQSTSSLSASLGDRVTISCRAS

SEQ ID NO: 739 - protein, 107 aa, Mus musculus
DIQMTQSTSSLSASLGDRVTISCRASQDISNYLNWYQQKPDGTVKLLIYFTSRLYSGVPSRFSGSGSGADYSLTISNLEQEDIATYFCQQGNTLPRTFGGGTKLEIK

SEQ ID NO: 740 - protein, 26 aa, Mus musculus
DIQMTQSTSSLSASLGDRVTITCRAS

SEQ ID NO: 741 - protein, 26 aa, Mus musculus
DIQMTQTHKFMSTSVGDRVSITCKAS

SEQ ID NO: 742 - protein, 107 aa, Mus musculus
DIQMTQTHKFMSTSVGDRVSITCKASQDVSTAVAWYQQKPGQSPKLLIYSASYRYTGVPDRFTGSGSGTDFTFTISSVQAEDLAVYYCQQHYSTHVHVRRGDQAGNQ

SEQ ID NO: 743 - protein, 26 aa, Mus musculus
DIQMTQTPASLAASVGETVTITCRAS

SEQ ID NO: 744 - protein, 26 aa, Mus musculus
DIQMTQTPASLSASVGETVTITCRAS

SEQ ID NO: 745 - protein, 107 aa, Mus musculus
DIQMTQTPASLSASVGETVTITCRASENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 746 - protein, 26 aa, Mus musculus
DIQMTQTPKFLLVSAGDRVTITCKAS

SEQ ID NO: 747 - protein, 107 aa, Mus musculus
DIQMTQTPKFLLVSAGDRVTITCKASQSVSNDVAWYQQKPGQSPKLLIYYASNRYTGVPDRFTGSGYGTDFTFTISTVQAEDLAVYFCQQDYSSPWTFGGGTKLEIK

SEQ ID NO: 748 - protein, 26 aa, Mus musculus
DIQMTQTPLTLSVTIGQPASISCKSS

SEQ ID NO: 749 - protein, 112 aa, Mus musculus
DIQMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGAHFPWTFGGGTKLEIK

SEQ ID NO: 750 - protein, 112 aa, Mus musculus
DIQMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEMK

SEQ ID NO: 751 - protein, 112 aa, Mus musculus
DIQMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPRTFGGGTKLEIK

SEQ ID NO: 752 - protein, 111 aa, Mus musculus
DIQMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTFSSHVRRGDQAGNE

SEQ ID NO: 753 - protein, 26 aa, Mus musculus
DIQMTQTPSSLSASLGERVSLTCRAS

SEQ ID NO: 754 - protein, 107 aa, Mus musculus
DIQMTQTPSSLSASLGERVSLTCRASQDIGSSLNWLQQEPDGTIKRLISATSSLDSGVPKRFSGSRSGSDYSLTISSLESEDFVDYYCLQYATFPYTFGGGTKLEIK

SEQ ID NO: 755 - protein, 107 aa, Mus musculus
DIQMTQTPSSLSASLGERVSLTCRASQDIGSSLNWLQQEPDGTIKRLIYATSSLDSGVPKRFSGSRSGSDYSLTISSLESEDFVDYYCLQYASSPRTFGGGTKLEIK

SEQ ID NO: 756 - protein, 107 aa, Mus musculus
DIQMTQTPSSLSASLGERVSLTCRASQEISGYLSWLQQKPDGTIKRLIYAASTLDSGVPKRFSGSRSGSDYSLTISSLESEDFADYYCLQYISYPRTFGGGTKLEIK

SEQ ID NO: 757 - protein, 26 aa, Mus musculus
DIQMTQTQKFMSTSVGDRVSVTCKAS

SEQ ID NO: 758 - protein, 26 aa, Mus musculus
DIQMTQTSSSFSVSLGDRVTITCKAS

SEQ ID NO: 759 - protein, 107 aa, Mus musculus
DIQMTQTSSSFSVSLGDRVTITCKASEDIYNRLAWYQQKPGNAPRLLISGATSLETGVPSRFSGSGSGKDYTLSITSLQTEDVATYYCQQYWSTPPTFGGGTKLEIK

SEQ ID NO: 760 - protein, 26 aa, Mus musculus
DIQMTQTTSSLSASLGDRVTISCRAS

SEQ ID NO: 761 - protein, 26 aa, Mus musculus
DIQMTQTTSSLSASLGDRVTISCSAS

SEQ ID NO: 762 - protein, 107 aa, Mus musculus
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPWTFGGGTKLEMK

SEQ ID NO: 763 - protein, 26 aa, Mus musculus
DIQMTQTTSSLSASLGDRVTITCRAS

SEQ ID NO: 764 - protein, 26 aa, Mus musculus
DIQMTRSPSSLSASLGERVSLTCRAS

SEQ ID NO: 765 - protein, 107 aa, Mus musculus
DIQMTRSPSSLSASLGERVSLTCRASQDIGSSLNWLQQEPDGTIKRLIYATSSLDSGVPKRFSGSRSGSDYSLTISSLESEDFVDYYCLQYASSPWTFGGGTKLEIK

SEQ ID NO: 766 - protein, 26 aa, Mus musculus
DIVITQSQKFMSTSVGDRVSVTCKAS

SEQ ID NO: 767 - protein, 107 aa, Mus musculus
DIVITQSQKFMSTSVGDRVSVTCKASQNVGSNVAWYQQKPGQSPKALIYSASYRYSGVPDRFTGSGSGTDFTLTISNVQSEDLAEYFCQQYDSYPYTFGGGTKLEIK

SEQ ID NO: 768 - protein, 26 aa, Mus musculus
DIVLTQSPASLAVSLGQRATISCRAS

SEQ ID NO: 769 - protein, 111 aa, Mus musculus
DIVLTQSPASLAVSLGQRATISCRASESVDNYGISFMNWFQKKPGQPPKLLIYAASNQGSGVPARFSGSGSGTDFSLNIHPMEEDDTAMYFCQQSKEVPYTFGGGTKLEIK

SEQ ID NO: 770 - protein, 26 aa, Mus musculus
DIVLTQSPASLSASVGESVTITCRAS

SEQ ID NO: 771 - protein, 26 aa, Mus musculus
DIVLTQSPPTLSLSPGERVTLSCKAS

SEQ ID NO: 772 - protein, 23 aa, Mus musculus
DIVLTQSQKFMSTSVGDRVSVTC

SEQ ID NO: 773 - protein, 26 aa, Mus musculus
DIVLTQSQKFMSTSVGDRVSVTCKAS

SEQ ID NO: 774 - protein, 26 aa, Mus musculus
DIVLTQSQKFMSTSVGDRVSVTCKAS

SEQ ID NO: 775 - protein, 107 aa, Mus musculus
DIVLTQSQKFMSTSVGDRVSVTCKASQNVGTNVAWYQQKPGQSPKALIYSASYRYSGVPDRFAGSGSGTDFTLTISNVQSEDLAEYFCQQYNSYPYTFGGGTKLEIK

SEQ ID NO: 776 - protein, 107 aa, Mus musculus
DIVLTQSQKFMSTSVGDRVSVTCKASQNVGTNVAWYQQKPGQSPKALIYSASYRYSGVPDRFTGSGSGTDFTLKISRVEAEDLGVYFCSQSTHVPYTFGGGTKLEIK

SEQ ID NO: 777 - protein, 107 aa, Mus musculus
DIVLTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEMK

SEQ ID NO: 778 - protein, 107 aa, Mus musculus
DIVLTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 779 - protein, 26 aa, Mus musculus
DIVMSQSHKFMSTSVGDRVSITCKAS

SEQ ID NO: 780 - protein, 107 aa, Mus musculus
DIVMSQSHKFMSTSVGDRVSITCKASQDVGTAVAWYQQKPGQSPKLLIYWASTRHTGVPDRFTGSGSGTDFTLTISIVQSEDLADYFCQQYSSYPLTFGGGTKLEIK

SEQ ID NO: 781 - protein, 107 aa, Mus musculus
DIVMSQSHKFMSTSVGDRVSITCKASQDVITAVAWYQQKPGQSPKLPIYSASYRYTGVPDRFTGSGSGTDFTFTISSVQAEDLAVYYCQQHYSTPRTFGGGTKLEIK

SEQ ID NO: 782 - protein, 26 aa, Mus musculus
DIVMSQSPAILSASPGEKVTMTCSAS

SEQ ID NO: 783 - protein, 106 aa, Mus musculus
DIVMSQSPAILSASPGEKVTMTCSASSRVTYMHWYQQKPGTSPKRWIYDTSTLTSGVPARFSGSGSGTSYSLTISGMEAEDAATYYCHQRSGYSYTFGGGTKLEMK

SEQ ID NO: 784 - protein, 26 aa, Mus musculus
DIVMSQSPAIMSASPGEKVTMTRSAS

SEQ ID NO: 785 - protein, 106 aa, Mus musculus
DIVMSQSPAIMSASPGEKVTMTRSASSSVSYMYWYQQKPGSSPRLLIYDTSNLASGVPVRFSGSGSGTSYSLTISRMEAEDAATYYCQQWSSYPYTSGGGTKLEIK

SEQ ID NO: 786 - protein, 26 aa, Mus musculus
DIVMSQSPASLAVSLGQRATISCKAS

SEQ ID NO: 787 - protein, 111 aa, Mus musculus
DIVMSQSPASLAVSLGQRATISCKASQSVDYDGDSYMNWYQQKPGQPPKLLIYATSNLESGIPARFSGSGSGTDFTLNIHPVEEEDAATYYCQQSNEDPLTFGAGTKLEIK

SEQ ID NO: 788 - protein, 26 aa, Mus musculus
DIVMSQSPASLSVATGEKVTIRCITS

SEQ ID NO: 789 - protein, 107 aa, Mus musculus
DIVMSQSPASLSVATGEKVTIRCITSTDIDDDMNWYQQKPGEPPKLLISEGNTLRPGVPSRFSSSGYGTDFVFTIENTLSEDVADYYCLQSDNMPYTFGGGTKLEIK

SEQ ID NO: 790 - protein, 26 aa, Mus musculus
DIVMSQSPSSLAVSVGEKVTMSCKSS

SEQ ID NO: 791 - protein, 111 aa, Mus musculus
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPFGGGTKLEIK

SEQ ID NO: 792 - protein, 23 aa, Mus musculus
DIVMSQSQKFMSTSVGDRVSVTC

SEQ ID NO: 793 - protein, 26 aa, Mus musculus
DIVMSQSQKFMSTSVGDRVSVTCKAS

SEQ ID NO: 794 - protein, 107 aa, Mus musculus
DIVMSQSQKFMSTSVGDRVSVTCKASQNVGTNVAWYQQKPGQSPKALIYSASYRYSGVPDRFTGSGSGTDFTLTISNVQSEDLAEYFCQQYNSYPRTFGGGTKLEIK

SEQ ID NO: 795 - protein, 107 aa, Mus musculus
DIVMSQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 796 - protein, 26 aa, Mus musculus
DIVMTQAAPSVPVTPGESVSISCRSS

SEQ ID NO: 797 - protein, 112 aa, Mus musculus
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPYTFGGGTKLEIK

SEQ ID NO: 798 - protein, 26 aa, Mus musculus
DIVMTQDELSNPVTSGESVSISCRSS

SEQ ID NO: 799 - protein, 112 aa, Mus musculus
DIVMTQDELSNPVTSGESVSISCRSSKSLLYKDGKTYLNCFLQRPGQSPQLLIYLMSTRASGVSNRFSGSGSGTDFTLEISRVKAEDVGVYYCHQLVEYPYTFGGGTKLEMK

SEQ ID NO: 800 - protein, 26 aa, Mus musculus
DIVMTQSHKFMSTSVGDRVSITCKAS

SEQ ID NO: 801 - protein, 107 aa, Mus musculus
DIVMTQSHKFMSTSVGDRVSITCKASQDVSTAVAWYQQKPGQSPKLLIHSASYRYTGVPDRFTGSGSGTDFTFTISSVQAEDLAVYYCQQHYSTPPTFGGGTKLEIK

SEQ ID NO: 802 - protein, 26 aa, Mus musculus
DIVMTQSPAIMSASPGEKVTMTCSAS

SEQ ID NO: 803 - protein, 106 aa, Mus musculus
DIVMTQSPAIMSASPGEKVTMTCSASSSVSYMHWYQQKSGTSPKRWIYDTSKLASGVPARFSGSGSGTSYSLTISSMEAEDAATYYCQQWSSNPPTFGGGTKLEIK

SEQ ID NO: 804 - protein, 107 aa, Mus musculus
DIVMTQSPAIMSASPGEKVTMTCSASSSVSYMHWYQQKSGTSPKRWIYDTSKLASGVPARFSGSGSGTSYSLTISSMEAEDAATYYCQQWSSNPPYTFGGGTKLEMK

SEQ ID NO: 805 - protein, 26 aa, Mus musculus
DIVMTQSPALMSASPGEKVTMTCSAS

SEQ ID NO: 806 - protein, 106 aa, Mus musculus
DIVMTQSPALMSASPGEKVTMTCSASSSVSYMYWYQQKPRSSPKPWIYLTPNLASGVPARFSGSGSGTSYSLTISSMEAEDAATYYCQQWSSNPYTFGGGTKLEIK

SEQ ID NO: 807 - protein, 26 aa, Mus musculus
DIVMTQSPASLSASVGETVTITCRAS

SEQ ID NO: 808 - protein, 26 aa, Mus musculus
DIVMTQSPKSMSMSVGERVTLSCKAS

SEQ ID NO: 809 - protein, 107 aa, Mus musculus
DIVMTQSPKSMSMSVGERVTLSCKASENVATYVSWYQQKPEQSPKLLIYGASNRYTGVPDRFTGSGSATDFTLTISSVQAEDLADYRCGQSYRYPYTFGGGTKLEMK

SEQ ID NO: 810 - protein, 107 aa, Mus musculus
DIVMTQSPKSMSMSVGERVTLSCKASENVGTYVSWYQQKPEQSPKLLIYGASNRYTGVPDRFTGSGSATDFTLTISSVQAEDLADYHCGQSYSYPYTFGGGTKLEIK

SEQ ID NO: 811 - protein, 107 aa, Mus musculus
DIVMTQSPKSMSMSVGERVTLSCKASENVGTYVSWYQQKPEQSPKLLIYGASNRYTGVPDRFTGSGSATDFTLTISSVQAEDLADYHCGQSYSYPWTFGGGTKLEIK

SEQ ID NO: 812 - protein, 26 aa, Mus musculus
DIVMTQSPLSLSVTIGQPASISCKSS

SEQ ID NO: 813 - protein, 113 aa, Mus musculus
DIVMTQSPLSLSVTIGQPASISCKSSQSLLYSNGKTYLNWLQQRPGQAPKHLMYQVSKLDPGIPDRFSGSGSETDFTLKISRVEAEDLGVYYCLQGTYYPTWTFGGGTKLEIK

SEQ ID NO: 814 - protein, 26 aa, Mus musculus
DIVMTQSPLTLSVTIGQPASISCKSS

SEQ ID NO: 815 - protein, 112 aa, Mus musculus
DIVMTQSPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPWTFGGGTKLEIK

SEQ ID NO: 816 - protein, 26 aa, Mus musculus
DIVMTQSPPTLSLSPGERVTLSCKAS

SEQ ID NO: 817 - protein, 26 aa, Mus musculus
DIVMTQSPSPLSASLGERVSLTCRAS

SEQ ID NO: 818 - protein, 107 aa, Mus musculus
DIVMTQSPSPLSASLGERVSLTCRASQEISGYLSWLQQKPDGTIKRLIYAASTLDSGVPKRFSGSRSGSDYSLTISSLESEDFADYYCLQYASYPRTFGGGTKLEIK

SEQ ID NO: 819 - protein, 23 aa, Mus musculus
DIVMTQSQKFMSTSVGDRVSVTC

SEQ ID NO: 820 - protein, 23 aa, Mus musculus
DIVMTQSQKFMSTSVGDRVSVTC

SEQ ID NO: 821 - protein, 26 aa, Mus musculus
DIVMTQSQKFMSTSVGDRVSVTCKAS

SEQ ID NO: 822 - protein, 107 aa, Mus musculus
DIVMTQSQKFMSTSVGDRVSVTCKASQNVGINVAWYQQKPGQSPKALIYSASYRYSGVPDRFTGSGSGTDFTLTISNVQSEDLAEYFCQQYNSYPYTFGGGTKLEIK

SEQ ID NO: 823 - protein, 107 aa, Mus musculus
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 824 - protein, 107 aa, Mus musculus
DIVMTQSQKFMSTSVGDRVSVTCKASQNVGTNVAWYQQKPGQSPKALIYSASYRYSGVPDRFTGSGSGTDFTLTISNVQSEDLAEYLCQQYNSYPRTFGGGTKLEIK

SEQ ID NO: 825 - protein, 107 aa, Mus musculus; identical to SEQ ID NO: 823

SEQ ID NO: 826 - protein, 26 aa, Mus musculus
DIVMTQSQKFMSTSVGDRVTITCKAS

SEQ ID NO: 827 - protein, 107 aa, Mus musculus
DIVMTQSQKFMSTSVGDRVTITCKASQNVRTAVAWYHQKPGQSPKALIYMASNRHTGVPDRFTGSGSGADFTLTITNVQSEDLADYFCLQHWNYPYTFGGGTKLEIK

SEQ ID NO: 828 - protein, 26 aa, Mus musculus
DIVMTQSTSSLSASLGDRVTISCRAS

SEQ ID NO: 829 - protein, 107 aa, Mus musculus
DIVMTQSTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 830 - protein, 26 aa, Mus musculus
DIVMTQTPKFLLVSAGDRVTITCKAS

SEQ ID NO: 831 - protein, 26 aa, Mus musculus
DIVMTQTTSSLSASLGDRVTISCRAS

SEQ ID NO: 832 - protein, 26 aa, Mus musculus
  VARIANT 3: note = Xaa can be any naturally occurring amino acid
  VARIANT 19: note = Xaa can be any naturally occurring amino acid
DIXMTQSTSSLSASLGDRXTITCRAS

SEQ ID NO: 833 - protein, 6 aa, Mus musculus
DNIYNY

SEQ ID NO: 834 - protein, 26 aa, Mus musculus
DNVLTQSPSSLAVSVGEKVTMSCKSS

SEQ ID NO: 835 - protein, 112 aa, Mus musculus
DNVLTQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYRTFGGGTKLEIK

SEQ ID NO: 836 - protein, 23 aa, Mus musculus
DNVLTQSQKFMSTSVGDRVSVTC

SEQ ID NO: 837 - protein, 26 aa, Mus musculus
DNVLTQSQKFMSTSVGDRVSVTCKAS

SEQ ID NO: 838 - protein, 107 aa, Mus musculus
DNVLTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEMK

SEQ ID NO: 839 - skipped

SEQ ID NO: 840 - protein, 26 aa, Mus musculus
DTTVTQSHKFMSTSVGDRVSITCKAS

SEQ ID NO: 841 - protein, 107 aa, Mus musculus
DTTVTQSHKFMSTSVGDRVSITCKASQDVSTAVAWYQQKPGQSPKLLIYSASYRYTGVPDRFTGSGSGTDFTFTISSVQAEDLAVYYCQQHYSTPWTFGGGTKLEIK

SEQ ID NO: 842 - protein, 107 aa, Mus musculus
DTTVTQSHKFMSTSVGDRVSITCKASQDVSTAVAWYQQKPGQSPKLLIYSASYRYTGVPDRFTGSGSGTDFTFTISSVQAEDLAVYYCQQHYSTPYTFGGGTKLEIK

SEQ ID NO: 843 - protein, 26 aa, Mus musculus
DTTVTQSHRFMSTSVEDRVTIACKAS

SEQ ID NO: 844 - protein, 107 aa, Mus musculus
DTTVTQSHRFMSTSVEDRVTIACKASQDVNTAVAWYQHKPGQSPKLLIYSASYRYTGVPDRFTGSGSRTDFTFTISSVQAEDLAVYYCQQHFNSPYTFGGGTKLEIK

SEQ ID NO: 845 - protein, 26 aa, Mus musculus
DTTVTQSPAIMSASPGEKVTMTCSAS

SEQ ID NO: 846 - protein, 106 aa, Mus musculus
DTTVTQSPAIMSASPGEKVTMTCSASSSIGYMHWYQQKPGTSPKRWIYDTSKLASGVPARFSGSGSGTSYSLTISSMEAEDAATYYCHQRGSYPWTFGGGTKLEIK

SEQ ID NO: 847 - protein, 26 aa, Mus musculus
DTTVTQSPASLAVSLGQRATISCKAS

SEQ ID NO: 848 - protein, 111 aa, Mus musculus
DTTVTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEMK

SEQ ID NO: 849 - protein, 111 aa, Mus musculus
DTTVTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIK

SEQ ID NO: 850 - protein, 26 aa, Mus musculus
DTTVTQSPASLAVSLGQRATISCRAS

SEQ ID NO: 851 - protein, 111 aa, Mus musculus
DTTVTQSPASLAVSLGQRATISCRASESVDSYGNSFMHWYQQKPGQPPKLLIYRASNLESGIPARFSGSGSRTGFTLTINPVEADDVATYYCQQSNEDPRTFGGGTKLEMK

SEQ ID NO: 852 - protein, 26 aa, Mus musculus
DTTVTQSPSSLAMSVGQKVTMSCKSS

SEQ ID NO: 853 - protein, 113 aa, Mus musculus
DTTVTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSTPYTFGGGTKLEIK

SEQ ID NO: 854 - protein, 26 aa, Mus musculus
DTTVTQSQKFMSTSVGDRVSVACKAS

SEQ ID NO: 855 - protein, 23 aa, Mus musculus
DTTVTQSQKFMSTSVGDRVSVTC

SEQ ID NO: 856 - protein, 26 aa, Mus musculus
DTTVTQSQKFMSTSVGDRVSVTCKAG

SEQ ID NO: 857 - protein, 107 aa, Mus musculus
DTTVTQSQKFMSTSVGDRVSVTCKAGQNVGTNVAWYQQKPGQSPKALIYSASYRYSGVPDRFTGSGSGTDFTLTISNVQSEDLAEYFCQQYNSYPYTFGGGTKLEIK

SEQ ID NO: 858 - protein, 26 aa, Mus musculus
DTTVTQSQKFMSTSVGDRVSVTCKAS

SEQ ID NO: 859 - protein, 107 aa, Mus musculus
DTTVTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 860 - protein, 107 aa, Mus musculus
DTTVTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEMK

SEQ ID NO: 861 - protein, 107 aa, Mus musculus
DTTVTQSQKFMSTSVGDRVSVTCKASQNVGTNVAWYQQKPGQSPKALIYSASYRYSGVPDRFTGSGSGTDFTLTISNVQSEDLAEYFCQQYNSYPWTFGGGTKLEIK

SEQ ID NO: 862 - protein, 23 aa, Mus musculus
DTTVTQSQRFMSTSVGDRVSVTC

SEQ ID NO: 863 - protein, 26 aa, Mus musculus
DTTVTQSQRFMSTSVGDRVSVTCKAS

SEQ ID NO: 864 - protein, 107 aa, Mus musculus
DTTVTQSQRFMSTSVGDRVSVTCKASQNVGTNVAWYQQKPGQSPKPLIYSASYRYSGVPDRFTGSGSGTDFTLTINNVQSEDLAEYFCQQYNNSPLTFGGGTKLEIK

SEQ ID NO: 865 - protein, 26 aa, Mus musculus
DVHMNQSQKFMSTSVGNRVSVTCKGS

SEQ ID NO: 866 - protein, 25 aa, Mus musculus
DVQLQESGAELAKPGASVKMSCKAS

SEQ ID NO: 867 - protein, 25 aa, Mus musculus
DVQLQQSGAELVRPGTSVKMSCKAA

SEQ ID NO: 868 - protein, 114 aa, Mus musculus
DVQLQQSGAELVRPGTSVKMSCKAAGYTFTNYWIGWVKQRPGHGLEWIGDIHPGGDYSNYNEKFKGKATLTADTSSSTAYMNLSSLTSEDSAIYYCTSRNFAYWGQGTLVTVSA

SEQ ID NO: 869 - protein, 114 aa, Mus musculus
DVQLQQSGAELVRPGTSVKMSCKAAGYTFTNYWIGWVKQRPGHGLEWIGDIHPGGGYTNYNEKFKGKATLTADTSSSTAYMQLSSLTSEGSAIYYCTSRNFAYWGQGTLVTVSA

SEQ ID NO: 870 - protein, 26 aa, Mus musculus
DVQMNQSPSSMYASLGERVTITCKAS

SEQ ID NO: 871 - protein, 26 aa, Mus musculus
DVQMTQSPASLSASVGETVTITCRAS

SEQ ID NO: 872 - protein, 26 aa, Mus musculus
DVQMTQSPSSMYASLGERVTFTCKAS

SEQ ID NO: 873 - protein, 26 aa, Mus musculus
DVQMTQSQKFMSTSVGDRVSVTCKAS

SEQ ID NO: 874 - protein, 26 aa, Mus musculus
DVVLTQTPLSLPVSLGDQASISCRSS

SEQ ID NO: 875 - protein, 112 aa, Mus musculus
DVVLTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 876 - protein, 112 aa, Mus musculus
DVVLTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 877 - protein, 112 aa, Mus musculus
DVVLTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEMK

SEQ ID NO: 878 - protein, 112 aa, Mus musculus
DVVLTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPRTFGGGTKLEIK

SEQ ID NO: 879 - protein, 111 aa, Mus musculus
DVVLTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPTFGGGTKLEIK

SEQ ID NO: 880 - protein, 112 aa, Mus musculus
DVVLTQTPLSLPVSLGDQASISCRSSQSLVHSYGNTYLHWYLQKPGQSPKLLIYKVSNRFSGVPDRFSGSGSGTDFTLKISRVEAEDLGVYFCSQSTHVPHTFGGGTKLEMK

SEQ ID NO: 881 - protein, 26 aa, Mus musculus
DVVLTQTPLSLPVSLGDQVSISCRSG

SEQ ID NO: 882 - protein, 112 aa, Mus musculus
DVVLTQTPLSLPVSLGDQVSISCRSGQSLVHNNGNTYLHWYLQKPGQSPKLLIYKVSNRFSGVPDRFSGSGSGTDFTLKISRVEAEDLGVYFCSQSTHVPLTFGAGTKLEIK

SEQ ID NO: 883 - protein, 26 aa, Mus musculus
DVVLTQTPLSLPVTLGDPASISCRSS

SEQ ID NO: 884 - protein, 112 aa, Mus musculus
DVVLTQTPLSLPVTLGDPASISCRSSQTLLHSDGNTYLKWYLQKPGQSPKLLIYQVSNRFSGVPDRFTGSGSGTDFTLKISRVEAEDLGVYHCFQGSHVPWTFGGGTKLEIK

SEQ ID NO: 885 - protein, 26 aa, Mus musculus
DVVMTQSHKFMSTSVGDRVSITCKAS

SEQ ID NO: 886 - protein, 108 aa, Mus musculus
DVVMTQSHKFMSTSVGDRVSITCKASQDVSTAVAWYQQKPGQSPKLLIYSASYRYTGVPDRFTGSGSGTDFTFTISSVQAEDLAVYYCQQHYSTPPWTFGRGTKLEIK

SEQ ID NO: 887 - protein, 26 aa, Mus musculus
DVVMTQSPAIMSASLGERVTMTCTAS

SEQ ID NO: 888 - protein, 108 aa, Mus musculus
DVVMTQSPAIMSASLGERVTMTCTASSSVSSSYLHWYQQKPGSSPKLWIYSTSNLASGVPARFSGSGSGTSYSLTISSMEAEDAATYYCHQYHRSPPTFGGGTKLEIK

SEQ ID NO: 889 - protein, 26 aa, Mus musculus
DVVMTQSPASLSASVGESVTITCRAS

SEQ ID NO: 890 - protein, 26 aa, Mus musculus
DVVMTQSPASLSASVGETVTITCRAS

SEQ ID NO: 891 - protein, 26 aa, Mus musculus
DVVMTQSQKFMSTSVGDRVNVTCKAS

SEQ ID NO: 892 - protein, 107 aa, Mus musculus
DVVMTQSQKFMSTSVGDRVNVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 893 - protein, 23 aa, Mus musculus
DVVMTQSQKFMSTSVGDRVSVTC

SEQ ID NO: 894 - protein, 26 aa, Mus musculus
DVVMTQSQKFMSTSVGDRVSVTCKAS

SEQ ID NO: 895 - protein, 107 aa, Mus musculus
DVVMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 896 - protein, 26 aa, Mus musculus
DVVMTQTPKFLLVSAGDRVTITCKAS

SEQ ID NO: 897 - protein, 107 aa, Mus musculus
DVVMTQTPKFLLVSAGDRVTITCKASQSVSNDVAWYQQKPGQSPKLLIYYASNRYTGVPDRFTGSGYGTDFTFTISTVQAEDLAVYFCQQDYSSPPTFGGGTKLEIK

SEQ ID NO: 898 - protein, 26 aa, Mus musculus
DVVMTQTPLSLPVNLGDQASISCRSS

SEQ ID NO: 899 - protein, 26 aa, Mus musculus
DVVMTQTPLSLPVSLGDQASISCRSN

SEQ ID NO: 900 - protein, 26 aa, Mus musculus
DVVMTQTPLSLPVSLGDQASISCRSS

SEQ ID NO: 901 - protein, 112 aa, Mus musculus
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 902 - protein, 112 aa, Mus musculus
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPLTFGAGTKLEIK

SEQ ID NO: 903 - protein, 112 aa, Mus musculus
DVVMTQTPLSLPVSLGDQASISCRSSQSLVHSNGNTYLRWYLQKPGQSPKLLIYKVSNRFSGVPDRFSGSGSGTDFTLKISRVEAEDLGVYFCSQSTHVPWTFGGGTKLEIK

SEQ ID NO: 904 - protein, 26 aa, Mus musculus
DVVMTQTPLTLSVTIGQPASISCKSS

SEQ ID NO: 905 - protein, 112 aa, Mus musculus
DVVMTQTPLTLSVTIGQPASISCKSSQSLLDSDGKTYLNWLLQRPGQSPKRLIYLVSKLDSGVPDRFTGSGSGTDFTLKINRVEAEDLGVYYCWQGTHFPRTFGGGTKLEIK

SEQ ID NO: 906 - protein, 111 aa, Mus musculus
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFRTFGGGTKLEMK

SEQ ID NO: 907 - protein, 112 aa, Mus musculus
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPWTFGGGTKLEIK

SEQ ID NO: 908 - protein, 112 aa, Mus musculus
DVVMTQTPLTLSVTIGQPASISCKSSQSLLDSDGKTYLNWLLQRPGQSPKRLIYLVSKLDSGVPDGFTGSGSGTDFTLKISRVEAEDLGVYYCWQGTHFPRTFGGGTKLEIK

SEQ ID NO: 909 - protein, 26 aa, Mus musculus
DVVMTQTQKFMSTSVGDRVSVTCKAS

SEQ ID NO: 910 - protein, 23 aa, Mus musculus
DVVMTQTQKFTSTSVGDRVSVTC

SEQ ID NO: 911 - protein, 26 aa, Mus musculus
DVVMTQTQKFTSTSVGDRVSVTCKAS

SEQ ID NO: 912 - protein, 107 aa, Mus musculus
DVVMTQTQKFTSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIK

SEQ ID NO: 913 - protein, 26 aa, Mus musculus
DVVMTQTTSSLSASLGDRVTISCRAS

SEQ ID NO: 914 - protein, 107 aa, Mus musculus
DVVMTQTTSSLSASLGDRVTISCRASQDISNYLNWYQQRPDGTVKLLIYYTSRLHSGVPSRFSGSGSGTDFSLTISNLEQEDIATYFCQQVYTLPWTFGGGTKLEIK

SEQ ID NO: 915 - protein, 26 aa, Mus musculus
DVVMTQTTSSLSASLGDRVTITCRAS

SEQ ID NO: 916 - protein, 26 aa, Mus musculus
DVVVTQSPASLAVSLGQRATISCRAS

SEQ ID NO: 917 - protein, 111 aa, Mus musculus
DVVVTQSPASLAVSLGQRATISCRASESVDSYGNSFMHWYQQKPGQPPKLLIYLASNLESGVPARFSGSGSRTDFTLTIDPVEADDAATYYCQQNNEDPYTFGGGTKLEIK

SEQ ID NO: 918 - protein, 26 aa, Mus musculus
DVVVTQTPLSLPVSLGDQASISCRSS

SEQ ID NO: 919 - protein, 112 aa, Mus musculus
DVVVTQTPLSLPVSLGDQASISCRSSQSLENSNGNTYLNWYLQKPGQSPQLLIYRVSNRFSGVLDRFSGSGSGTDFTLKISRVEAEDLGVYFCLQVTHVPFAFGSGTKLEIK

SEQ ID NO: 920 - protein, 111 aa, Mus musculus
DVVVTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPTFGGGTKLEIK

SEQ ID NO: 921 - protein, 111 aa, Mus musculus
DVVVTQTPLSLPVSLGDQASISCRSSQSLVHSNGNTYLHWYLQKPGQSPKLLIYKVSNRFSGVPDRFSGSGSGTDFTLKISRVDAEDLGVYFCSQSTHVYTFGGGTKLEMK

SEQ ID NO: 922 - protein, 112 aa, Mus musculus
DVVVTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 923 - protein, 38 aa, Mus musculus
DYNEKFTGKATLTADTSSSTAYMQLSSLTSEDSAIYYC

SEQ ID NO: 924 - protein, 6 aa, Mus musculus
EDIYNR

SEQ ID NO: 925 - skipped

SEQ ID NO: 926 - protein, 26 aa, Mus musculus
EIVMTQSPPTLSLSPGERVTLSCKAS

SEQ ID NO: 927 - protein, 6 aa, Mus musculus
ENIYFS

SEQ ID NO: 928 - protein, 6 aa, Mus musculus
ENIYSF

SEQ ID NO: 929 - protein, 6 aa, Mus musculus
ENIYSN

SEQ ID NO: 930 - protein, 6 aa, Mus musculus
ENIYSY

SEQ ID NO: 931 - protein, 6 aa, Mus musculus
ENSYSY

SEQ ID NO: 932 - protein, 6 aa, Mus musculus
ENVATY

SEQ ID NO: 933 - protein, 6 aa, Mus musculus
ENVGTY

SEQ ID NO: 934 - protein, 10 aa, Mus musculus
ESVDNYGISF

SEQ ID NO: 935 - protein, 10 aa, Mus musculus
ESVDSYGNSF

SEQ ID NO: 936 - protein, 25 aa, Mus musculus
EVHLQQSGAELVRSGASVKLSCTAS

SEQ ID NO: 937 - protein, 25 aa, Mus musculus
EVLLQQFGAELVRSGASVKLSCTAS

SEQ ID NO: 938 - protein, 25 aa, Mus musculus
EVLLQQSGADLMKPGASVKISCKAS

SEQ ID NO: 939 - protein, 25 aa, Mus musculus
EVQLQESGADLMKPGASVKISCKAS

SEQ ID NO: 940 - protein, 25 aa, Mus musculus
EVQLQQSGAELVRPGVSLKISCKGS

SEQ ID NO: 941 - protein, 25 aa, Mus musculus
EVRLQQSGADLMKPGASVKISCKTS

SEQ ID NO: 942 - protein, 25 aa, Mus musculus
EVRLQQSGAELVRSGASVKLPCTAS

SEQ ID NO: 943 - protein, 25 aa, Mus musculus
EVRLQQSGAELVRSGASVKLSCTAS

SEQ ID NO: 944 - skipped

SEQ ID NO: 945 - protein, 37 aa, Mus musculus
EYAPKFGKATMTADTSSNTAHLQLSSLTSEDTAVYYC

SEQ ID NO: 946 - protein, 38 aa, Mus musculus
EYAPKFQGKATMTADTSSNTAHLQLSSLTSEDTAVYYC

SEQ ID NO: 947 - protein, 38 aa, Mus musculus
EYAPKFQGKATMTADTSSNTAYLQLSSLTSEDTAVYYC

SEQ ID NO: 948 - protein, 38 aa, Mus musculus
EYAPKFQGKATMTTDTSSNTAHLQLSSLTSEDTAVYYC

SEQ ID NO: 949 - protein, 37 aa, Mus musculus
EYNEKFDKATLTADKSSSTAYMQLSSLTSEDSAVYFC

SEQ ID NO: 950 - protein, 38 aa, Mus musculus
EYNEKFEDKATLTADKSSSTAYMQLSSLTSEDSAVYYC

SEQ ID NO: 951 - protein, 37 aa, Mus musculus
EYNQKFDKATLTADKSSSTAYMQLSSLTSDDSAVYYC

SEQ ID NO: 952 - protein, 37 aa, Mus musculus
  VARIANT 25: note = Xaa can be any naturally occurring amino acid
EYNQKFDKATLTADKSSSTAYMQLXSLTSDDSAVYYC

SEQ ID NO: 953 - protein, 38 aa, Mus musculus
EYNQKFEDKATLTADKSSNTAYMQLNSLTSEDSAVYYC

SEQ ID NO: 954 - protein, 38 aa, Mus musculus
EYNQKFEDKATLTADKSSNTAYMQLSSLTSEDSAVYYC

SEQ ID NO: 955 - protein, 38 aa, Mus musculus
EYNQKFKDKATLTADKSSSTAYMQLSSLTSDDSAVYYC

SEQ ID NO: 956 - protein, 12 aa, Mus musculus
FAFGSGTKLEIK

SEQ ID NO: 957 - skipped

SEQ ID NO: 958 - protein, 10 aa, Mus musculus
FGAGTKLEIK

SEQ ID NO: 959 - protein, 10 aa, Mus musculus
FGGGTKLEIK

SEQ ID NO: 960 - protein, 10 aa, Mus musculus
FGGGTKLEIK

SEQ ID NO: 961 - protein, 10 aa, Mus musculus
FGGGTKLEMK

SEQ ID NO: 962 - protein, 10 aa, Mus musculus
FGGGTKLEMK

SEQ ID NO: 963 - protein, 10 aa, Mus musculus
  VARIANT 6: note = Xaa can be any naturally occurring amino acid
FGGGTXLEIK

SEQ ID NO: 964 - protein, 10 aa, Mus musculus
FGRGTKLEIK

SEQ ID NO: 965 - protein, 10 aa, Mus musculus
FGSGTKLEIK

SEQ ID NO: 966 - protein, 9 aa, Mus musculus
FQGSHVPPT

SEQ ID NO: 967 - protein, 9 aa, Mus musculus
FQGSHVPRT

SEQ ID NO: 968 - protein, 9 aa, Mus musculus
FQGSHVPTF

SEQ ID NO: 969 - protein, 9 aa, Mus musculus
FQGSHVPWT

SEQ ID NO: 970 - protein, 9 aa, Mus musculus
FQGSHVPYT

SEQ ID NO: 971 - protein, 9 aa, Mus musculus
FQGSYVPRT

SEQ ID NO: 972 - skipped

SEQ ID NO: 973 - protein, 8 aa, Mus musculus
FYPGGDYI

SEQ ID NO: 974 - protein, 9 aa, Mus musculus
GAGTKLEIK

SEQ ID NO: 975-976 - skipped (2 entries)

SEQ ID NO: 977 - protein, 8 aa, Mus musculus
GFNIKDYY

SEQ ID NO: 978 - protein, 10 aa, Mus musculus
GGGTKKLEIK

SEQ ID NO: 979 - protein, 9 aa, Mus musculus
GQSYRYPYT

SEQ ID NO: 980 - protein, 9 aa, Mus musculus
GQSYSYPWT

SEQ ID NO: 981 - protein, 9 aa, Mus musculus
GQSYSYPYT

SEQ ID NO: 982 - protein, 6 aa, Mus musculus
GRNFAY

SEQ ID NO: 983 - skipped

SEQ ID NO: 984 - protein, 8 aa, Mus musculus
GYAFTNYW

SEQ ID NO: 985 - protein, 7 aa, Mus musculus
GYTFSNY

SEQ ID NO: 986 - protein, 8 aa, Mus musculus
GYTFSNYW

SEQ ID NO: 987 - protein, 8 aa, Mus musculus
GYTFTDYG

SEQ ID NO: 988 - protein, 8 aa, Mus musculus
GYTFTDYS

SEQ ID NO: 989 - protein, 7 aa, Mus musculus
GYTFTKY

SEQ ID NO: 990 - protein, 7 aa, Mus musculus
GYTFTNY

SEQ ID NO: 991 - protein, 8 aa, Mus musculus
GYTFTRFW

SEQ ID NO: 992 - protein, 8 aa, Mus musculus
GYTFTRYW

SEQ ID NO: 993 - protein, 8 aa, Mus musculus
GYTFTTST

SEQ ID NO: 994 - protein, 8 aa, Mus musculus
GYTSTAYW

SEQ ID NO: 995 - protein, 8 aa, Mus musculus
GYTSTGYW

SEQ ID NO: 996 - protein, 7 aa, Mus musculus
GYTSTNY

SEQ ID NO: 997 - protein, 8 aa, Mus musculus
GYTSTNYW

SEQ ID NO: 998 - protein, 9 aa, Mus musculus
HQLVEYPYT

SEQ ID NO: 999 - protein, 9 aa, Mus musculus
HQRGSYPWT

SEQ ID NO: 1000 - protein, 9 aa, Mus musculus
HQRSGYSYT

SEQ ID NO: 1001 - protein, 9 aa, Mus musculus
HQYHRSPPT

SEQ ID NO: 1002 - skipped

SEQ ID NO: 1003 - protein, 8 aa, Mus musculus
IDPDNGET

SEQ ID NO: 1004 - protein, 8 aa, Mus musculus
IDPENGDT

SEQ ID NO: 1005 - protein, 17 aa, Mus musculus
IEWIKQRPGHGLEWIGE

SEQ ID NO: 1006 - protein, 17 aa, Mus musculus
IEWIKQRPGHGLEWVGE

SEQ ID NO: 1007 - protein, 17 aa, Mus musculus
IGWIKQRPGHGLEWIGD

SEQ ID NO: 1008 - protein, 17 aa, Mus musculus
IGWVKLRPGHGLEWIGD

SEQ ID NO: 1009 - protein, 17 aa, Mus musculus
IGWVKQRPGHDLEWIGD

SEQ ID NO: 1010 - protein, 17 aa, Mus musculus
IGWVKQRPGHGLEWIGD

SEQ ID NO: 1011 - protein, 17 aa, Mus musculus
IGWVKQRPGHGLEWVGD

SEQ ID NO: 1012 - protein, 8 aa, Mus musculus
IHPGGDYS

SEQ ID NO: 1013 - protein, 8 aa, Mus musculus
IHPGGDYT

SEQ ID NO: 1014 - protein, 8 aa, Mus musculus
IHPGGGYI

SEQ ID NO: 1015 - protein, 8 aa, Mus musculus
IHPGGGYT

SEQ ID NO: 1016 - protein, 8 aa, Mus musculus
IHPGGSYT

SEQ ID NO: 1017 - protein, 17 aa, Mus musculus
IHWVKQRPEQGLEWIGW

SEQ ID NO: 1018 - protein, 17 aa, Mus musculus
IHWVKQRPGQDLEWIGY

SEQ ID NO: 1019 - protein, 17 aa, Mus musculus
IHWVKQRPGQDLEWIGY

SEQ ID NO: 1020 - protein, 8 aa, Mus musculus
IITYSGDA

SEQ ID NO: 1021 - protein, 8 aa, Mus musculus
ILPGSGFT

SEQ ID NO: 1022 - protein, 8 aa, Mus musculus
ILPGSGYT

SEQ ID NO: 1023 - protein, 8 aa, Mus musculus
INPGGGYT

SEQ ID NO: 1024 - protein, 8 aa, Mus musculus
INPRSGYT

SEQ ID NO: 1025 - protein, 8 aa, Mus musculus
INPRTDYT

SEQ ID NO: 1026 - protein, 8 aa, Mus musculus
INPSSDYT

SEQ ID NO: 1027 - protein, 8 aa, Mus musculus
INPSTDYT

SEQ ID NO: 1028 - protein, 8 aa, Mus musculus
ITPSTGYT

SEQ ID NO: 1029 - protein, 8 aa, Mus musculus
  VARIANT 2: note = Xaa can be any naturally occurring amino acid
IXTYSGDV

SEQ ID NO: 1030 - protein, 37 aa, Mus musculus
IYNQKFDKATMTVDKSSSTAYLELARLTSEDSAIYYC

SEQ ID NO: 1031 - protein, 37 aa, Mus musculus
IYNQKFGKATMTVDKSSSTAYLELARLTSDDSAIYYC

SEQ ID NO: 1032 - protein, 37 aa, Mus musculus
IYNQKFGKATMTVDKSSSTAYLELARLTSEDSAIYYC

SEQ ID NO: 1033 - protein, 38 aa, Mus musculus
IYNQKFKGKATMTVDKSSSTAYLELARLTSDDSAIYYC

SEQ ID NO: 1034 - protein, 38 aa, Mus musculus
IYNQKFKGKATMTVDKSSSTAYLELARLTSEDSAIYYC

SEQ ID NO: 1035 - protein, 8 aa, Mus musculus
IYPGGGYA

SEQ ID NO: 1036 - protein, 11 aa, Mus musculus
KAGQNVGTNVA

SEQ ID NO: 1037 - protein, 11 aa, Mus musculus
KASQNVGINVA

SEQ ID NO: 1038 - protein, 11 aa, Mus musculus
KASQNVGTNVA

SEQ ID NO: 1039 - protein, 11 aa, Mus musculus
KASQNVGTNVA

SEQ ID NO: 1040 - protein, 11 aa, Mus musculus
KASQSVGTNVA

SEQ ID NO: 1041 - protein, 31 aa, Mus musculus
KATFTADTSSSTTYMQLSSLTSEDSAIYFCT

SEQ ID NO: 1042 - protein, 31 aa, Mus musculus
KATLTADTFSSTAYMQLSSLTSEDSAIYYCT

SEQ ID NO: 1043 - protein, 31 aa, Mus musculus
KATLTADTSSSTADMQLSSLTSEDSAIYYCT

SEQ ID NO: 1044 - protein, 31 aa, Mus musculus
KATLTADTSSSTAYMNLSSLTSEDSAIYYCT

SEQ ID NO: 1045 - protein, 31 aa, Mus musculus
KATLTADTSSSTAYMQLSSLTSEDSAIYYCT

SEQ ID NO: 1046 - protein, 31 aa, Mus musculus
KATLTADTSSSTAYMQLSSLTSEDSAIYYCV

SEQ ID NO: 1047 - protein, 31 aa, Mus musculus
KATLTADTSSSTAYMQLSSLTSEGSAIYYCT

SEQ ID NO: 1048 - protein, 31 aa, Mus musculus
KATLTADTSSSTAYMQLSSLTSGDSAIYYCV

SEQ ID NO: 1049 - protein, 31 aa, Mus musculus
KATLTADTSSSTAYMSLSSLTSEDSAIYYCT

SEQ ID NO: 1050 - protein, 31 aa, Mus musculus
KATLTAGTSSSTAYMQLSSLTSEDSAIYYCV

SEQ ID NO: 1051 - protein, 36 aa, Mus musculus
KLASGVPARFSGSGSGTSYSLSISSMETEDAATYYC

SEQ ID NO: 1052 - protein, 36 aa, Mus musculus
KLASGVPARFSGSGSGTSYSLTISSMEAEDAATYYC

SEQ ID NO: 1053 - protein, 36 aa, Mus musculus
KLDPGIPDRFSGSGSETDFTLKISRVEAEDLGVYYC

SEQ ID NO: 1054 - protein, 36 aa, Mus musculus
KLDSGVPDGFTGSGSGTDFTLKISRVEAEDLGVYYC

SEQ ID NO: 1055 - protein, 36 aa, Mus musculus
KLDSGVPDRFTGSGSGTDFTLKINRVEAEDLGVYYC

SEQ ID NO: 1056 - protein, 36 aa, Mus musculus
KLDSGVPDRFTGSGSGTDFTLKISRVEAEDLGVYYC

SEQ ID NO: 1057 - protein, 6 aa, Mus musculus
KNSYSY

SEQ ID NO: 1058 - protein, 9 aa, Mus musculus
KQAYDVPWT

SEQ ID NO: 1059 - protein, 6 aa, Mus musculus
KSISKY

SEQ ID NO: 1060 - protein, 11 aa, Mus musculus
KSLLHSNGNTY

SEQ ID NO: 1061 - protein, 11 aa, Mus musculus
KSLLYKDGKTY

SEQ ID NO: 1062-1063 - skipped (2 entries)

SEQ ID NO: 1064 - protein, 17 aa, Mus musculus
LAWYQEKPGKTNKLLIY

SEQ ID NO: 1065 - protein, 17 aa, Mus musculus
LAWYQQKPGNAPRLLIS

SEQ ID NO: 1066 - protein, 17 aa, Mus musculus
LAWYQQKPGQSPKLLIY

SEQ ID NO: 1067 - protein, 17 aa, Mus musculus
LAWYQQKPGQSPKLLVY

SEQ ID NO: 1068 - protein, 17 aa, Mus musculus
LAWYQQKQGKSPQLLIY

SEQ ID NO: 1069 - protein, 17 aa, Mus musculus
LAWYQQKQGKSPQLLVY

SEQ ID NO: 1070 - protein, 17 aa, Mus musculus
LEWFQQKQGKSPQLLVY

SEQ ID NO: 1071 - protein, 17 aa, Mus musculus
LEWYLQKPGQSPKLLIY

SEQ ID NO: 1072 - protein, 17 aa, Mus musculus
LEWYQQKQGKSPQLLVY

SEQ ID NO: 1073 - protein, 17 aa, Mus musculus
LEWYQQKQGKSPQLLVY

SEQ ID NO: 1074 - protein, 17 aa, Mus musculus
LGWIKQRPGHGLEWIGD

SEQ ID NO: 1075 - protein, 17 aa, Mus musculus
LGWVKQRPGHGLEWIGD

SEQ ID NO: 1076 - protein, 17 aa, Mus musculus
LHWYLQKPGQSPKLLIY

SEQ ID NO: 1077 - protein, 17 aa, Mus musculus
LHWYQQKPGSSPKLWIY

SEQ ID NO: 1078 - protein, 17 aa, Mus musculus
LHWYQQKSETSPKPWIY

SEQ ID NO: 1079 - protein, 17 aa, Mus musculus
LHWYQQKSHESPRLLIK

SEQ ID NO: 1080 - protein, 17 aa, Mus musculus
LKWYLQKPGQSPKLLIY

SEQ ID NO: 1081 - skipped

SEQ ID NO: 1082 - protein, 17 aa, Mus musculus
LNCFLQRPGQSPQLLIY

SEQ ID NO: 1083 - protein, 17 aa, Mus musculus
LNWLLQRPGQSPKRLIY

SEQ ID NO: 1084 - protein, 17 aa, Mus musculus
LNWLQQEPDGTIKRLIS

SEQ ID NO: 1085 - protein, 17 aa, Mus musculus
LNWLQQEPDGTIKRLIY

SEQ ID NO: 1086 - protein, 17 aa, Mus musculus
LNWLQQRPGQAPKHLMY

SEQ ID NO: 1087 - protein, 17 aa, Mus musculus
LNWYLQKPGQSPQLLIY

SEQ ID NO: 1088 - protein, 17 aa, Mus musculus
LNWYQQKPDGTVKLLIY

SEQ ID NO: 1089 - protein, 17 aa, Mus musculus
LNWYQQKSDGTVKLLIY

SEQ ID NO: 1090 - protein, 17 aa, Mus musculus
LNWYQQRPDGTVKLLIY

SEQ ID NO: 1091 - protein, 16 aa, Mus musculus
LNYQQKPDGTVKLLIY

SEQ ID NO: 1092 - protein, 10 aa, Mus musculus
LQGTYYPTWT

SEQ ID NO: 1093 - protein, 9 aa, Mus musculus
LQHWNYPYT

SEQ ID NO: 1094 - protein, 9 aa, Mus musculus
LQSDNMPLT

SEQ ID NO: 1095 - protein, 9 aa, Mus musculus
LQSDNMPYT

SEQ ID NO: 1096 - protein, 9 aa, Mus musculus
LQVTHVPFA

SEQ ID NO: 1097 - protein, 9 aa, Mus musculus
LQYASSPFT

SEQ ID NO: 1098 - protein, 9 aa, Mus musculus
LQYASSPHT

SEQ ID NO: 1099 - protein, 9 aa, Mus musculus
LQYASSPRT

SEQ ID NO: 1100 - protein, 9 aa, Mus musculus
LQYASSPWT

SEQ ID NO: 1101 - protein, 9 aa, Mus musculus
LQYASSPYT

SEQ ID NO: 1102 - protein, 9 aa, Mus musculus
LQYASYPRT

SEQ ID NO: 1103 - protein, 9 aa, Mus musculus
LQYASYPYT

SEQ ID NO: 1104 - protein, 9 aa, Mus musculus
LQYATFPYT

SEQ ID NO: 1105 - protein, 9 aa, Mus musculus
LQYDEFPWT

SEQ ID NO: 1106 - protein, 9 aa, Mus musculus
LQYDEFPYT

SEQ ID NO: 1107 - protein, 9 aa, Mus musculus
LQYISYPRT

SEQ ID NO: 1108 - protein, 17 aa, Mus musculus
LRWYLQKPGQSPKLLIY

SEQ ID NO: 1109 - protein, 17 aa, Mus musculus
LSWFQQKPGKSPKTLIY

SEQ ID NO: 1110 - protein, 17 aa, Mus musculus
LSWFQQKPGKSPQTLIY

SEQ ID NO: 1111 - protein, 17 aa, Mus musculus
LSWLQQKPDGTIKRLIY

SEQ ID NO: 1112 - protein, 17 aa, Mus musculus
LSWLQQKPDGTIKRLTY

SEQ ID NO: 1113 - protein, 17 aa, Mus musculus
LSWYQQKPDGTIKLLIY

SEQ ID NO: 1114 - protein, 12 aa, Mus musculus
LTFGAGTKLEIK

SEQ ID NO: 1115-1116 - skipped (2 entries)

SEQ ID NO: 1117 - protein, 17 aa, Mus musculus
LVWYQQKQGKSPHLLVY

SEQ ID NO: 1118 - protein, 37 aa, Mus musculus
LYNQKFGKATMTVDKSSSTAYLELARLTSDDSAIYYC

SEQ ID NO: 1119 - protein, 38 aa, Mus musculus
LYNQKFKGKATMTVDKSSSTAYLELARLTSDDSAIYYC

SEQ ID NO: 1120 - protein, 38 aa, Mus musculus
LYNQKFKGKATMTVDKSSSTAYLELARLTSEDSAIYYC

SEQ ID NO: 1121 - protein, 17 aa, Mus musculus
LYWFLQRPGQSPQLLIY

SEQ ID NO: 1122 - skipped

SEQ ID NO: 1123 - protein, 17 aa, Mus musculus
MHWVKQRHGQGLEWIGN

SEQ ID NO: 1124 - protein, 17 aa, Mus musculus
MHWVKQRPEQGLEWIGW

SEQ ID NO: 1125 - protein, 17 aa, Mus musculus
MHWVKQRPGQDLEWIGY

SEQ ID NO: 1126 - protein, 17 aa, Mus musculus
MHWVKQSHAKSLEWIGV

SEQ ID NO: 1127 - protein, 17 aa, Mus musculus
MHWVRQSHAKNLEWIGV

SEQ ID NO: 1128 - protein, 17 aa, Mus musculus
MHWVRQSHAKSLEWIGV

SEQ ID NO: 1129 - protein, 17 aa, Mus musculus
MHWYQQKPGQPPKLLIY

SEQ ID NO: 1130 - protein, 17 aa, Mus musculus
MHWYQQKPGTSPKRWIY

SEQ ID NO: 1131 - protein, 17 aa, Mus musculus
MHWYQQKSGTSPKRWIY

SEQ ID NO: 1132 - protein, 26 aa, Mus musculus
MLVMTQTPLSLPVSLGDQASISCRSS

SEQ ID NO: 1133 - protein, 17 aa, Mus musculus
MNWFQKKPGQPPKLLIY

SEQ ID NO: 1134 - protein, 17 aa, Mus musculus
MNWYQQKPGEPPKLLIS

SEQ ID NO: 1135 - protein, 17 aa, Mus musculus
MNWYQQKPGQPPKLLIY

SEQ ID NO: 1136 - protein, 9 aa, Mus musculus
MQHLEYPYT

SEQ ID NO: 1137 - protein, 17 aa, Mus musculus
MQWVRQSHAKSLEWIGV

SEQ ID NO: 1138 - protein, 17 aa, Mus musculus
MRWVKQSHAKSLEWIGV

SEQ ID NO: 1139 - protein, 17 aa, Mus musculus
  VARIANT 10: note = Xaa can be any naturally occurring amino acid
MRWVKQSPAXSLEWIGV

SEQ ID NO: 1140 - protein, 17 aa, Mus musculus
MYWYQQKPGSSPRLLIY

SEQ ID NO: 1141 - protein, 17 aa, Mus musculus
MYWYQQKPRSSPKPWIY

SEQ ID NO: 1142 - skipped

SEQ ID NO: 1143 - protein, 36 aa, Mus musculus
NLADGVPSRFSGSGSGTQYSLKINSLQSEDFGSYYC

SEQ ID NO: 1144 - protein, 36 aa, Mus musculus
NLASGVPARFSGSGSGTSYSLTISSMEAEDAATYYC

SEQ ID NO: 1145 - protein, 36 aa, Mus musculus
NLASGVPDRFSGSGSGTAFTLRISRVEAEDVGVYYC

SEQ ID NO: 1146 - protein, 36 aa, Mus musculus
NLASGVPVRFSGSGSGTSYSLTISRMEAEDAATYYC

SEQ ID NO: 1147 - protein, 36 aa, Mus musculus
NLASGVPVRFSGSGSGTSYSLTISSMEAEDAATYYC

SEQ ID NO: 1148 - protein, 36 aa, Mus musculus
NLESGIPARFSGSGSGTDFTLNIHPVEEEDAATYYC

SEQ ID NO: 1149 - protein, 36 aa, Mus musculus
NLESGIPARFSGSGSRTGFTLTINPVEADDVATYYC

SEQ ID NO: 1150 - protein, 36 aa, Mus musculus
NLESGVPARFSGSGSRTDFTLTIDPVEADDAATYYC

SEQ ID NO: 1151 - protein, 36 aa, Mus musculus
NLQSGVPSRFTGSGSGTDYSLTISNLEQEDIATYFC

SEQ ID NO: 1152 - protein, 36 aa, Mus musculus
NQGSGVPARFSGSGSGTDFSLNIHPMEEDDTAMYFC

SEQ ID NO: 1153 - protein, 36 aa, Mus musculus
NRFSGVLDRFSGSGSGTDFTLKISRVEAEDLGVYFC

SEQ ID NO: 1154 - protein, 36 aa, Mus musculus
NRFSGVPDRFSGSGSGTDFTLKISKVEAEDLGVYYC

SEQ ID NO: 1155 - protein, 36 aa, Mus musculus
NRFSGVPDRFSGSGSGTDFTLKISRVDAEDLGVYFC

SEQ ID NO: 1156 - protein, 36 aa, Mus musculus
NRFSGVPDRFSGSGSGTDFTLKISRVEAEDLGIYYC

SEQ ID NO: 1157 - protein, 36 aa, Mus musculus [sequence omitted]

SEQ ID NO: 1158 - protein, 36 aa, Mus musculus
NRFSGVPDRFSGSGSGTDFTLKISRVEAEDLGVYYC

SEQ ID NO: 1159 - protein, 36 aa, Mus musculus
NRFSGVPDRFSVSGSGTDFTLKISRVEAEDLGVYFC

SEQ ID NO: 1160 - protein, 36 aa, Mus musculus
NRFSGVPDRFTGSGSGTDFTLKISRVEAEDLGVYHC

SEQ ID NO: 1161 - protein, 36 aa, Mus musculus
NRHTGVPDRFTGSGSGADFTLTITNVQSEDLADYFC

SEQ ID NO: 1162 - protein, 36 aa, Mus musculus
NRYTGVPDRFTGSGSATDFTLTISSVQAEDLADYHC

SEQ ID NO: 1163 - protein, 36 aa, Mus musculus
NRYTGVPDRFTGSGSATDFTLTISSVQAEDLADYRC

SEQ ID NO: 1164 - protein, 36 aa, Mus musculus
NRYTGVPDRFTGSGYGTDFTFTISTVQAEDLAVYFC

SEQ ID NO: 1165 - protein, 12 aa, Mus musculus
NVITTATTWFAY

SEQ ID NO: 1166 - protein, 38 aa, Mus musculus
NYDEKFKSKGTLTVDTSSSTAYMHLSSLASEDSAVYYC

SEQ ID NO: 1167 - protein, 38 aa, Mus musculus
NYDEKFKSKGTLTVDTSSSTAYMHLSSLTSEDSAVYCC

SEQ ID NO: 1168 - protein, 38 aa, Mus musculus
NYNEKFEGKATLTADTSSSTAYMQLSSLTSEDSAIYYC

SEQ ID NO: 1169 - protein, 38 aa, Mus musculus
NYNEKFEGKATLTADTSSSTAYMQLSSLTSEGSAIYYC

SEQ ID NO: 1170 - protein, 37 aa, Mus musculus
NYNEKFGKATLTADTSSSTAYMQLSSLTSEDSAVYFC

SEQ ID NO: 1171 - protein, 38 aa, Mus musculus
NYNEKFKGKATLTADTFSSTAYMQLSSLTSEDSAIYYC

SEQ ID NO: 1172 - protein, 38 aa, Mus musculus
NYNEKFKGKATLTADTSPSTAYMQLSSLTSEDSAVYFC

SEQ ID NO: 1173 - protein, 38 aa, Mus musculus
NYNEKFKGKATLTADTSSSTADMQLSSLTSEDSAIYYC

SEQ ID NO: 1174 - protein, 38 aa, Mus musculus
NYNEKFKGKATLTADTSSSTAYMNLSSLTSEDSAIYYC

SEQ ID NO: 1175 - protein, 38 aa, Mus musculus
NYNEKFKGKATLTADTSSSTAYMNLSSLTSGDSAIYYC

SEQ ID NO: 1176 - protein, 38 aa, Mus musculus
NYNEKFKGKATLTADTSSSTAYMQLNSLTSEDSAVYFC

SEQ ID NO: 1177 - protein, 38 aa, Mus musculus
NYNEKFKGKATLTADTSSSTAYMQLSSLTSEDSAIYYC

SEQ ID NO: 1178 - protein, 38 aa, Mus musculus
NYNEKFKGKATLTADTSSSTAYMQLSSLTSEDSAVYFC

SEQ ID NO: 1179 - protein, 38 aa, Mus musculus
NYNEKFKGKATLTADTSSSTAYMQLSSLTSEGSAIYYC

SEQ ID NO: 1180 - protein, 38 aa, Mus musculus
NYNEKFKGKATLTADTSSSTAYMSLSSLTSEDSAIYYC

SEQ ID NO: 1181 - protein, 38 aa, Mus musculus
NYNEKFKGKATLTADTSSSTTYMQLSSLTSEDSAIYYC

SEQ ID NO: 1182 - protein, 37 aa, Mus musculus
NYNEKFKKATLTADTSSSTAYMQLSSLTSEDSAVYFC

SEQ ID NO: 1183 - protein, 37 aa, Mus musculus
NYNEKFKKATLTADTSSVTAYMQLSSLTSEDSAVYFC

SEQ ID NO: 1184 - protein, 38 aa, Mus musculus
NYNEKFTGKATLTADTSSSTAYMQLSSLTSEDSAIYYC

SEQ ID NO: 1185 - protein, 37 aa, Mus musculus
NYNENFGKATFTADTSSNTTYMQLSSLTSEDSAVYYC

SEQ ID NO: 1186 - protein, 38 aa, Mus musculus
NYNENFKGKATFTADTSSNTTYIQLSSLSSEDSAVYYC

SEQ ID NO: 1187 - protein, 38 aa, Mus musculus
NYNENFKGKATFTADTSSNTTYMLLSSLSSEDSAVYYC

SEQ ID NO: 1188 - protein, 38 aa, Mus musculus
NYNENFKGKATFTADTSSNTTYMQLSSLTSEDSAVYYC

SEQ ID NO: 1189 - protein, 38 aa, Mus musculus
NYNENFKGKATFTADTSSSTTYMQLSSLTSEDSAIYFC

SEQ ID NO: 1190 - protein, 37 aa, Mus musculus
NYNQKFGKATMTVDKSSSTAYMELARLTSEDSAIYYC

SEQ ID NO: 1191 - protein, 25 aa, Mus musculus
PGPTQQPGSELVRPGASVKLSCKAS

SEQ ID NO: 1192 - protein, 25 aa, Mus musculus
PIQLQQSGAELVRPGASVKLSCKAS

SEQ ID NO: 1193 - protein, 25 aa, Mus musculus
QAYLQQSGAELVRPGTSVKISCKAS

SEQ ID NO: 1194 - protein, 25 aa, Mus musculus
QAYLQQSGAELVRPGTSVKMSCKAA

SEQ ID NO: 1195 - protein, 25 aa, Mus musculus
QAYLQQSGAELVRPGVSLKISCKGS

SEQ ID NO: 1196 - protein, 25 aa, Mus musculus
QAYLQQSGAELVRPGVSVKISCKGS

SEQ ID NO: 1197 - protein, 25 aa, Mus musculus
QAYLQQSGAELVRSGASVKLSCTAS

SEQ ID NO: 1198 - protein, 6 aa, Mus musculus
QDIGLN

SEQ ID NO: 1199 - protein, 6 aa, Mus musculus
QDIGSS

SEQ ID NO: 1200 - protein, 6 aa, Mus musculus
QDINNF

SEQ ID NO: 1201 - protein, 6 aa, Mus musculus
QDINRY

SEQ ID NO: 1202 - protein, 6 aa, Mus musculus
QDIRNY

SEQ ID NO: 1203 - protein, 6 aa, Mus musculus
QDISNY

SEQ ID NO: 1204 - protein, 6 aa, Mus musculus
QDVGTA

SEQ ID NO: 1205 - protein, 6 aa, Mus musculus
QDVITA

SEQ ID NO: 1206 - protein, 6 aa, Mus musculus
QDVNTA

SEQ ID NO: 1207 - protein, 8 aa, Mus musculus
QDVNTAVA

SEQ ID NO: 1208 - protein, 6 aa, Mus musculus
QDVRTA

SEQ ID NO: 1209 - protein, 6 aa, Mus musculus
QDVSTA

SEQ ID NO: 1210 - protein, 6 aa, Mus musculus
QEISGY

SEQ ID NO: 1211 - protein, 6 aa, Mus musculus
QGISNY

SEQ ID NO: 1212 - protein, 9 aa, Mus musculus
QHFWGTPPT

SEQ ID NO: 1213 - protein, 9 aa, Mus musculus
QHFWGTPWT

SEQ ID NO: 1214 - protein, 9 aa, Mus musculus
QHHYGIPYT

SEQ ID NO: 1215 - protein, 9 aa, Mus musculus
QHHYGSPWT

SEQ ID NO: 1216 - protein, 9 aa, Mus musculus
QHHYGSPYT

SEQ ID NO: 1217 - protein, 9 aa, Mus musculus
QHHYGTPWT

SEQ ID NO: 1218 - protein, 25 aa, Mus musculus
QIQLQQPGAELAKPGASVKMSCKAS

SEQ ID NO: 1219 - protein, 25 aa, Mus musculus
QIQLQQPGAELVKPGASVKLPCKAS

SEQ ID NO: 1220 - protein, 25 aa, Mus musculus
QIQLQQPGAELVRPGTSVKISCKAS

SEQ ID NO: 1221 - protein, 25 aa, Mus musculus
QIQLQQPGAELVRPGTSVKMSCKAA

SEQ ID NO: 1222 - protein, 114 aa, Mus musculus
QIQLQQPGAELVRPGTSVKMSCKAAGYTFTKYWIGWVKQRPGHGLEWIGDIHPGGGYINYNEKFTGKATLTAGTSSSTAYMQLSSLTSEDSAIYYCVSRNFANWGQGTLVTVSA

SEQ ID NO: 1223 - protein, 25 aa, Mus musculus
QIQLQQPGAELVRPGVSLKISCKGS

SEQ ID NO: 1224 - protein, 25 aa, Mus musculus
QIQLQQSGADLMKPGASVKISCKAS

SEQ ID NO: 1225 - protein, 25 aa, Mus musculus
QIQLQQSGAELAKPGASVKMSCKAS

SEQ ID NO: 1226 - protein, 25 aa, Mus musculus
QIQLQQSGAELVRPGTSLKMSCKAA

SEQ ID NO: 1227 - protein, 25 aa, Mus musculus
QIQLQQSGAELVRPGTSVKISCKAS

SEQ ID NO: 1228 - protein, 25 aa, Mus musculus
QIQLQQSGAELVRPGTSVKMSCKAA

SEQ ID NO: 1229 - protein, 114 aa, Mus musculus
QIQLQQSGAELVRPGTSVKMSCKAAGYTFSNYWIGWVKQRPGHGLEWIGDIHPGGGYINYNEKFTGKATLTADTSSSTAYMQLSSLTSEDSAIYYCVSRNFANWGQGTLVTVSA

SEQ ID NO: 1230 - protein, 114 aa, Mus musculus
QIQLQQSGAELVRPGTSVKMSCKAAGYTFTKYWIGWVKQRSGHGLEWIGDIHPGGGYINYNEKFTGKATLTADTSSSTAYMQLSSLTSEDSAIYYCVSRNFANWGQGTLVTVSA

SEQ ID NO: 1231 - protein, 114 aa, Mus musculus
QIQLQQSGAELVRPGTSVKMSCKAAGYTFTNYWIGWVKQRPGHGLEWIGDFYPGGDYINYNEKFKGKATLTADTSSSTAYMQLSSLTSEDSAIYYCTSRNFAYWGQGTPVTVSS

SEQ ID NO: 1232 - protein, 114 aa, Mus musculus
QIQLQQSGAELVRPGTSVKMSCKAAGYTFTNYWIGWVKQRPGHGLEWIGDIHPGGDYSNYNEKFKGKATLTADTSSSTAYMNLSSLTSEDSAIYYCTSRNFAYWGQGTLVTVSS

SEQ ID NO: 1233 - protein, 114 aa, Mus musculus
QIQLQQSGAELVRPGTSVKMSCKAAGYTFTNYWIGWVKQRPGHGLEWIGDIHPGGDYSNYNEKFKGKATLTADTSSSTAYMSLSSLTSEDSAIYYCTSRNFAYWGQGTLVTVSA

SEQ ID NO: 1234 - protein, 114 aa, Mus musculus
QIQLQQSGAELVRPGTSVKMSCKAAGYTFTNYWIGWVKQRPGHGLEWIGDIHPGGGYTNYNEKFKGKATLTADTSSSTADMQLSSLTSEDSAIYYCTSRNFAYWGQGTLVTVSA

SEQ ID NO: 1235 - protein, 114 aa, Mus musculus
QIQLQQSGAELVRPGTSVKMSCKAAGYTFTNYWIGWVKQRPGHGLEWIGDIHPGGGYTNYNEKFKGKATLTADTSSSTAYMQLSSLTSEDSAIYYCTSRNFAYWGQGTLVTVSA

SEQ ID NO: 1236 - protein, 114 aa, Mus musculus
QIQLQQSGAELVRPGTSVKMSCKAAGYTFTNYWIGWVKQRPGHGLEWIGDIHPGGGYTNYNEKFKGKATLTADTSSSTAYMQLSSLTSEDSAIYYCTSRNFAYWGQGTPVTVSS

SEQ ID NO: 1237 - protein, 114 aa, Mus musculus
QIQLQQSGAELVRPGTSVKMSCKAAGYTFTNYWIGWVKQRPGHGLEWIGDIHPGGSYTNYNENFKGKATFTADTSSSTTYMQLSSLTSEDSAIYFCTSRNFAKWGQGTPVTVSS

SEQ ID NO: 1238 - protein, 114 aa, Mus musculus
QIQLQQSGAELVRPGTSVKMSCKAAGYTSTNYWIGWVKQRPGHGLEWIGDIHPGGDYSNYNEKFKGKATLTADTSSSTAYMNLSSLTSEDSAIYYCTSRNFAYWGQGTLVTVSA

SEQ ID NO: 1239 - protein, 25 aa, Mus musculus
QIQLQQSGAELVRPGVSLKISCKGS

SEQ ID NO: 1240 - protein, 25 aa, Mus musculus
QIQLQQSGAELVRPGVSVKISCKGS

SEQ ID NO: 1241 - protein, 25 aa, Mus musculus
QIQLQQSGAEMVRPGVSVKISCKGS

SEQ ID NO: 1242 - protein, 9 aa, Mus musculus
QNGHSFPRT

SEQ ID NO: 1243 - protein, 6 aa, Mus musculus
QNVGIN

SEQ ID NO: 1244 - protein, 6 aa, Mus musculus
QNVGSN

SEQ ID NO: 1245 - protein, 6 aa, Mus musculus
QNVGTN

SEQ ID NO: 1246 - protein, 8 aa, Mus musculus
QNVGTNVA

SEQ ID NO: 1247 - protein, 6 aa, Mus musculus
QNVRTA

SEQ ID NO: 1248 - protein, 9 aa, Mus musculus
QQAYWSPYT

SEQ ID NO: 1249 - protein, 9 aa, Mus musculus
QQDYRSPYT

SEQ ID NO: 1250 - protein, 9 aa, Mus musculus
QQDYSSPPT

SEQ ID NO: 1251 - protein, 9 aa, Mus musculus
QQDYSSPWT

SEQ ID NO: 1252 - protein, 9 aa, Mus musculus
QQGHTLPPT

SEQ ID NO: 1253 - protein, 9 aa, Mus musculus
QQGNTLPPT

SEQ ID NO: 1254 - protein, 9 aa, Mus musculus
QQGNTLPRT

SEQ ID NO: 1255 - protein, 9 aa, Mus musculus
QQGNTLPWT

SEQ ID NO: 1256 - protein, 9 aa, Mus musculus
QQHFNSPYT

SEQ ID NO: 1257 - protein, 9 aa, Mus musculus
QQHNEYPWT

SEQ ID NO: 1258 - protein, 9 aa, Mus musculus
QQHYSSPWT

SEQ ID NO: 1259 - protein, 9 aa, Mus musculus
QQHYSTHVH

SEQ ID NO: 1260 - protein, 9 aa, Mus musculus
QQHYSTPFT

SEQ ID NO: 1261 - protein, 9 aa, Mus musculus
QQHYSTPPT

SEQ ID NO: 1262 - protein, 10 aa, Mus musculus
QQHYSTPPWT

SEQ ID NO: 1263 - protein, 9 aa, Mus musculus
QQHYSTPRT

SEQ ID NO: 1264 - protein, 9 aa, Mus musculus
QQHYSTPWT

SEQ ID NO: 1265 - protein, 9 aa, Mus musculus
QQHYSTPYT

SEQ ID NO: 1266 - protein, 9 aa, Mus musculus
QQNNEDPYT

SEQ ID NO: 1267 - protein, 9 aa, Mus musculus
QQSKEVPYT

SEQ ID NO: 1268 - protein, 9 aa, Mus musculus
QQSNEDPLT

SEQ ID NO: 1269 - protein, 9 aa, Mus musculus
QQSNEDPRT

SEQ ID NO: 1270 - protein, 9 aa, Mus musculus
QQSNEDPYT

SEQ ID NO: 1271 - protein, 9 aa, Mus musculus
QQSNSLPPT

SEQ ID NO: 1272 - protein, 9 aa, Mus musculus
QQSNTLPPT

SEQ ID NO: 1273 - protein, 9 aa, Mus musculus
QQVYTLPWT

SEQ ID NO: 1274 - protein, 9 aa, Mus musculus
QQWSSNPPT

SEQ ID NO: 1275 - protein, 10 aa, Mus musculus
QQWSSNPPYT

SEQ ID NO: 1276 - protein, 9 aa, Mus musculus
QQWSSNPYT

SEQ ID NO: 1277 - protein, 9 aa, Mus musculus
QQWSSYPLT

SEQ ID NO: 1278 - protein, 9 aa, Mus musculus
QQWSSYPYT

SEQ ID NO: 1279 - protein, 9 aa, Mus musculus
QQWTSNPPT

SEQ ID NO: 1280 - protein, 9 aa, Mus musculus
QQYDSYPYT

SEQ ID NO: 1281 - protein, 9 aa, Mus musculus
QQYNNHPYT

SEQ ID NO: 1282 - protein, 9 aa, Mus musculus
QQYNNSPLT

SEQ ID NO: 1283 - protein, 9 aa, Mus musculus
QQYNNYPLT

SEQ ID NO: 1284 - protein, 9 aa, Mus musculus
QQYNNYPWT

SEQ ID NO: 1285 - protein, 9 aa, Mus musculus
QQYNRYPYT

SEQ ID NO: 1286 - protein, 9 aa, Mus musculus
QQYNSYPRT

SEQ ID NO: 1287 - protein, 9 aa, Mus musculus
QQYNTYPYT

SEQ ID NO: 1288 - protein, 9 aa, Mus musculus
QQYNTYPYT

SEQ ID NO: 1289 - protein, 9 aa, Mus musculus
QQYSKLPWT

SEQ ID NO: 1290 - protein, 9 aa, Mus musculus
QQYSNYLTF

SEQ ID NO: 1291 - protein, 9 aa, Mus musculus
QQYSSYPLT

SEQ ID NO: 1292 - protein, 9 aa, Mus musculus
QQYWSTPPT

SEQ ID NO: 1293 - protein, 7 aa, Mus musculus
QQYYSYP

SEQ ID NO: 1294 - protein, 9 aa, Mus musculus
QQYYSYPWT

SEQ ID NO: 1295 - protein, 8 aa, Mus musculus
QQYYSYRT

SEQ ID NO: 1296 - protein, 6 aa, Mus musculus
QSISDY

SEQ ID NO: 1297 - protein, 36 aa, Mus musculus
QSISGIPSRFSGSGSGSDFTLSINSVEPEDVGVYYC

SEQ ID NO: 1298 - protein, 11 aa, Mus musculus
QSIVHNNGNTY

SEQ ID NO: 1299 - protein, 11 aa, Mus musculus
QSIVHSNGNTY

SEQ ID NO: 1300 - protein, 11 aa, Mus musculus
QSLENSNGNTY

SEQ ID NO: 1301 - protein, 11 aa, Mus musculus
QSLLDSDGKTY

SEQ ID NO: 1302 - protein, 12 aa, Mus musculus
QSLLNSSNQKNY

SEQ ID NO: 1303 - protein, 11 aa, Mus musculus
QSLLYSNGKTY

SEQ ID NO: 1304 - protein, 12 aa, Mus musculus
QSLLYSSNQKNY

SEQ ID NO: 1305 - protein, 11 aa, Mus musculus
QSLVHNNGNTY

SEQ ID NO: 1306 - protein, 11 aa, Mus musculus
QSLVHSNGNTY

SEQ ID NO: 1307 - protein, 11 aa, Mus musculus
QSLVHSYGNTY

SEQ ID NO: 1308 - protein, 10 aa, Mus musculus
QSVDYDGDSY

SEQ ID NO: 1309 - protein, 6 aa, Mus musculus
QSVNND

SEQ ID NO: 1310 - protein, 6 aa, Mus musculus
QSVSND

SEQ ID NO: 1311 - protein, 11 aa, Mus musculus
QTLLHSDGNTY

SEQ ID NO: 1312 - protein, 25 aa, Mus musculus
QVQLKQSGADLMKPGASVKISCKAS

SEQ ID NO: 1313 - protein, 24 aa, Mus musculus
QVQLQPGAELVRPGTSVKISCKAS

SEQ ID NO: 1314 - protein, 25 aa, Mus musculus
QVQLQQPGAELVRPGTSVKISCKAS

SEQ ID NO: 1315 - protein, 25 aa, Mus musculus
QVQLQQPGAELVRPGTSVKMSCKAA

SEQ ID NO: 1316 - protein, 114 aa, Mus musculus
QVQLQQPGAELVRPGTSVKMSCKAAGYTFSNYWIGWVKQRPGHGLEWIGDIHPGGGYINYNEKFTGKATLTADTSSSTAYMQLSSLTSEDSAIYYCVSRNFANWGQGTLVTVSA

SEQ ID NO: 1317 - protein, 114 aa, Mus musculus
QVQLQQPGAELVRPGTSVKMSCKAAGYTFSNYWIGWVKQRPGHGLEWIGDIHPGGGYINYNEKFTGKATLTADTSSSTAYMQLSSLTSEDSAIYYCVSRNFANWGQGTPVTVSS

SEQ ID NO: 1318 - protein, 114 aa, Mus musculus
QVQLQQPGAELVRPGTSVKMSCKAAGYTFTNYWIGWVKQRPGHGLEWIGDIHPGGDYSNYNEKFKGKATLTADTSSSTAYMNLSSLTSEDSAIYYCTSRNFAYWGQGTLVTVSA

SEQ ID NO: 1319 - protein, 25 aa, Mus musculus
QVQLQQPGAELVRPGVSLKISCKGS

SEQ ID NO: 1320 - protein, 25 aa, Mus musculus
QVQLQQPGAEMAKPGASVKMSCKAS

SEQ ID NO: 1321 - protein, 25 aa, Mus musculus
QVQLQQSGADLMKPGASVKMSCRAS

SEQ ID NO: 1322 - protein, 25 aa, Mus musculus
QVQLQQSGAELVRPGASVKISCKAS

SEQ ID NO: 1323 - protein, 25 aa, Mus musculus
QVQLQQSGAELVRPGISVKISCKGS

SEQ ID NO: 1324 - protein, 25 aa, Mus musculus
QVQLQQSGAELVRPGTSLKMSCKAA

SEQ ID NO: 1325 - protein, 25 aa, Mus musculus
QVQLQQSGAELVRPGTSVKISCKAS

SEQ ID NO: 1326 - protein, 25 aa, Mus musculus
QVQLQQSGAELVRPGTSVKMSCKAA

SEQ ID NO: 1327 - protein, 114 aa, Mus musculus
QVQLQQSGAELVRPGTSVKMSCKAAGYTFSNYWIGWVKQRPGHGLEWIGDIHPGGGYINYNEKFTGKATLTADTSSSTAYMQLSSLTSEDSAIYYCVSRNFANWGQGTLVTVSA

SEQ ID NO: 1328 - protein, 114 aa, Mus musculus
QVQLQQSGAELVRPGTSVKMSCKAAGYTFSNYWIGWVKQRPGRGLEWIGDIHPGGGYINYNEKFTGKATLTADTSSSTAYMQLSSLTSGDSAIYYCVSRNFANWGQGTLVTVSA

SEQ ID NO: 1329 - protein, 114 aa, Mus musculus
QVQLQQSGAELVRPGTSVKMSCKAAGYTFTNYWIGWIKQRPGHGLEWIGDIHPGGDYTNYNEKFKGKATLTADTFSSTAYMQLSSLTSEDSAIYYCTGRNFAYWGQGTLVTVSS

SEQ ID NO: 1330 - protein, 114 aa, Mus musculus
QVQLQQSGAELVRPGTSVKMSCKAAGYTFTNYWIGWVKQRPGHDLEWIGDIHPGGDYSNYNEKFKGKATLTADTSSSTAYMNLSSLTSEDSAIYYCTSRNFAYWGQGTLVTVSA

SEQ ID NO: 1331 - protein, 114 aa, Mus musculus
QVQLQQSGAELVRPGTSVKMSCKAAGYTFTNYWIGWVKQRPGHGLEWIGDFYPGGDYINYNEKFKGKATLTADTSSSTAYMQLSSLTSEDSAIYYCTSRNFAYWGQGTLVTVSA

SEQ ID NO: 1332 - protein, 114 aa, Mus musculus
QVQLQQSGAELVRPGTSVKMSCKAAGYTFTNYWIGWVKQRPGHGLEWIGDIHPGGDYSNYNEKFKGKATLTADTSSSTAYMNLSSLTSEDSAIYYCTSRNFAYWGQGTPVTVSS

SEQ ID NO: 1333 - protein, 114 aa, Mus musculus
QVQLQQSGAELVRPGTSVKMSCKAAGYTFTNYWIGWVKQRPGHGLEWIGDIHPGGDYSNYNEKFKGKATLTADTSSSTAYMNLSSLTSEDSAIYYCTSRNFAYWGQGTLVTVSA

SEQ ID NO: 1334 - protein, 114 aa, Mus musculus
QVQLQQSGAELVRPGTSVKMSCKAAGYTFTNYWIGWVKQRPGHGLEWIGDIHPGGGYIDYNEKFTGKATLTADTSSSTAYMQLSSLTSEDSAIYYCVSRNFAKWGQGTLVTVSA

SEQ ID NO: 1335 - protein, 114 aa, Mus musculus
QVQLQQSGAELVRPGTSVKMSCKAAGYTFTNYWIGWVKQRPGHGLEWIGDIHPGGGYTNYNEKFKGKATLTADTSSSTAYMQLSSLTSEDSAIYYCTSRNFAYWGQGTLVTVSA

SEQ ID NO: 1336 - protein, 25 aa, Mus musculus
QVQLQQSGAELVRPGTSVRMSCKAA

SEQ ID NO: 1337 - protein, 25 aa, Mus musculus
QVQLQQSGAELVRPGVSLKISCKGS

SEQ ID NO: 1338 - protein, 25 aa, Mus musculus
QVQLQQSGAELVRPGVSVKISCKGS

SEQ ID NO: 1339 - protein, 25 aa, Mus musculus
QVQLQQSGAELVRSGASVKLSCTAS

SEQ ID NO: 1340 - protein, 25 aa, Mus musculus
QVQLQQSGAEVAKPGASVKMSCKAS

SEQ ID NO: 1341 - protein, 25 aa, Mus musculus
QVQLQQSGAGLVRPGVSLKISCKGS

SEQ ID NO: 1342 - protein, 25 aa, Mus musculus
QVQLQQSGSELVRPGASVKLSCKAS

SEQ ID NO: 1343 - protein, 25 aa, Mus musculus
QVQMKQSGAELAKPGASVKMSCKAS

SEQ ID NO: 1344-1346 - skipped (3 entries)

SEQ ID NO: 1347 - protein, 26 aa, Mus musculus
RCPDDPSPSSLSASLGERVSLTCRAS

SEQ ID NO: 1348 - protein, 26 aa, Mus musculus
RCQMNQSPSSLSASLGERVSLTCRAS

SEQ ID NO: 1349 - protein, 26 aa, Mus musculus
RHCESQSHKFMSTSIGDRVSITCKAS

SEQ ID NO: 1350 - protein, 36 aa, Mus musculus
RLHSGVPSRFSGSESGTDYSLTISNLEQEDIATYFC

SEQ ID NO: 1351 - protein, 36 aa, Mus musculus
RLHSGVPSRFSGSGSGTDFSLTISNLEQEDIATYFC

SEQ ID NO: 1352 - protein, 36 aa, Mus musculus
RLHSGVPSRFSGSGSGTDYSLTISNLEQEDFANYFC

SEQ ID NO: 1353 - protein, 36 aa, Mus musculus
RLHSGVPSRFSGSGSGTDYSLTISNLEQEDIATYFC

SEQ ID NO: 1354 - protein, 36 aa, Mus musculus
RLHSGVSSRFSGSGSGTDYSLTISNLEQEDFATYFC

SEQ ID NO: 1355 - protein, 36 aa, Mus musculus
RLQSGVPSRFTGSGSGTDYSLTISNLEQEDIATYFC

SEQ ID NO: 1356 - protein, 36 aa, Mus musculus
RLVDGVPSRFSGSGSGQHYSLTISGLEYEDLGIYYC

SEQ ID NO: 1357 - protein, 36 aa, Mus musculus
RLVDGVPSRFSGSGSGQNYSLTISSLEYEDMGIYYC

SEQ ID NO: 1358 - protein, 36 aa, Mus musculus
RLYSGVPSRFSGSGSGADYSLTISNLEQEDIATYFC

SEQ ID NO: 1359 - protein, 36 aa, Mus musculus
RLYSGVPSRFSGSGSGTDYSLTISNLEQEDIATYFC

SEQ ID NO: 1360-1361 - skipped (2 entries)

SEQ ID NO: 1362 - protein, 8 aa, Mus musculus
SASYRYSG

SEQ ID NO: 1363 - protein, 8 aa, Mus musculus
SASYRYSG

SEQ ID NO: 1364 - protein, 10 aa, Mus musculus
SGGGTKLEIK

SEQ ID NO: 1365 - skipped

SEQ ID NO: 1366 - protein, 36 aa, Mus musculus
SLDSGVPKRFSGSRSGSDYSLTISSLESEDFVDYYC

SEQ ID NO: 1367 - protein, 36 aa, Mus musculus
SLETGVPSRFSGSGSGKDYTLSITSLQTEDVATYYC

SEQ ID NO: 1368 - protein, 36 aa, Mus musculus
SLHSGVPSRFSGSGSGTDYSLTISNLEPEDIATYYC

SEQ ID NO: 1369 - protein, 9 aa, Mus musculus
SQSTHVPHT

SEQ ID NO: 1370 - protein, 9 aa, Mus musculus
SQSTHVPLT

SEQ ID NO: 1371 - protein, 9 aa, Mus musculus
SQSTHVPPT

SEQ ID NO: 1372 - protein, 9 aa, Mus musculus
SQSTHVPRT

SEQ ID NO: 1373 - protein, 8 aa, Mus musculus
SQSTHVPT

SEQ ID NO: 1374 - protein, 9 aa, Mus musculus
SQSTHVPWT

SEQ ID NO: 1375 - protein, 9 aa, Mus musculus
SQSTHVPYT

SEQ ID NO: 1376 - protein, 8 aa, Mus musculus
SQSTHVYT

SEQ ID NO: 1377 - protein, 6 aa, Mus musculus
SRNFAK

SEQ ID NO: 1378 - protein, 6 aa, Mus musculus
SRNFAN

SEQ ID NO: 1379 - protein, 6 aa, Mus musculus
SRNFAY

SEQ ID NO: 1380 - protein, 5 aa, Mus musculus
SRVTY

SEQ ID NO: 1381 - protein, 5 aa, Mus musculus
SSIGY

SEQ ID NO: 1382 - protein, 7 aa, Mus musculus
SSISSSN

SEQ ID NO: 1383 - protein, 5 aa, Mus musculus
SSVIY

SEQ ID NO: 1384 - protein, 7 aa, Mus musculus
SSVSSSY

SEQ ID NO: 1385 - protein, 5 aa, Mus musculus
SSVSY

SEQ ID NO: 1386 - skipped

SEQ ID NO: 1387 - protein, 37 aa, Mus musculus
SYNQKFGKATMTVDKSSSTAYMELARLTSEDSAIYYC

SEQ ID NO: 1388 - protein, 38 aa, Mus musculus
SYNQKFKGKATMTVDKSSSTAYMELARLTPEDSAIYYC

SEQ ID NO: 1389 - protein, 38 aa, Mus musculus
SYNQKFKGKATMTVDKSSSTAYMELARLTSEDSAIYYC

SEQ ID NO: 1390 - protein, 38 aa, Mus musculus
SYNQKFKGKATMTVDKSSSTAYMELARLTSEDSATYYC

SEQ ID NO: 1391 - protein, 6 aa, Mus musculus
TDIDDD

SEQ ID NO: 1392 - protein, 17 aa, Mus musculus
TEWIKQRPGHGLEWIGE

SEQ ID NO: 1393 - protein, 11 aa, Mus musculus
TFGGGTKLEIK

SEQ ID NO: 1394 - protein, 11 aa, Mus musculus
TFGGGTKLEMK

SEQ ID NO: 1395 - protein, 7 aa, Mus musculus
TGRNFAY

SEQ ID NO: 1396 - protein, 36 aa, Mus musculus
TLAEGVPSRFSGIGSGTQFSLKINSLQPEDFGTYYC

SEQ ID NO: 1397 - protein, 36 aa, Mus musculus
TLAEGVPSRFSGSGSGTHFSLKINSLQPEDFGNYYC

SEQ ID NO: 1398 - protein, 36 aa, Mus musculus
TLAEGVPSRFSGSGSGTQFSLKINSLQPEDFGSYYC

SEQ ID NO: 1399 - protein, 36 aa, Mus musculus
TLAEGVPSRFSGSGSGTQFSLKINSLQPEDFGTYYC

SEQ ID NO: 1400 - protein, 36 aa, Mus musculus
TLAEGVPSRFSGSGSGTQFSLRINSLQPEDFGTYYC

SEQ ID NO: 1401 - protein, 36 aa, Mus musculus
TLAEGVPSRFSVSGSGTQFSLKINSLQPEDFGSYYC

SEQ ID NO: 1402 - protein, 36 aa, Mus musculus
TLDSGVPKRFSGSRSGSDYSLTISSLESEDFADYYC

SEQ ID NO: 1403 - protein, 36 aa, Mus musculus
TLEDGVPSRFSGSGSGTQFSMKINNMQPEDTATYFC

SEQ ID NO: 1404 - protein, 36 aa, Mus musculus
TLGEGVPSRFSGSGSGTQFYLKINSLQPEDFGSYYC

SEQ ID NO: 1405 - protein, 36 aa, Mus musculus
TLQSGIPSRFSGSGSGTDFTLTISSLEPEDFAMYYC

SEQ ID NO: 1406 - protein, 36 aa, Mus musculus
TLRPGVPSRFSSSGYGTDFVFTIENTLSEDVADYYC

SEQ ID NO: 1407 - protein, 36 aa, Mus musculus
TLTSGVPARFSGSGSGTSYSLTISGMEAEDAATYYC

SEQ ID NO: 1408 - protein, 36 aa, Mus musculus
TLVEGVPSRFSGSGSGTQFSLKINSLQPEDFGSYYC

SEQ ID NO: 1409 - protein, 36 aa, Mus musculus
TRASGVSNRFSGSGSGTDFTLEISRVKAEDVGVYYC

SEQ ID NO: 1410 - protein, 36 aa, Mus musculus
TRESGVPDRFIGSGSGTDFTLTISSVQAEDLADYFC

SEQ ID NO: 1411 - protein, 36 aa, Mus musculus
TRESGVPDRFTGSGSGTDFTLTISSVKAEDLAVYYC

SEQ ID NO: 1412 - protein, 36 aa, Mus musculus
TRHTGVPDRFTGSGSGTDFTLTISIVQSEDLADYFC

SEQ ID NO: 1413 - protein, 36 aa, Mus musculus
TRHTGVPSGFSGSGSGTDFTLTISSLQPEDFATYYC

SEQ ID NO: 1414 - protein, 36 aa, Mus musculus
TRHTGVPSRFSGSGSGTDFTLTISSLQPEDFATYYC

SEQ ID NO: 1415 - protein, 15 aa, Mus musculus
TRQNYYGSSHWYFDV

SEQ ID NO: 1416 - protein, 14 aa, Mus musculus
TRSGVEGLLHWYFD

SEQ ID NO: 1417 - protein, 12 aa, Mus musculus
TRWITTDHYFDY

SEQ ID NO: 1418 - protein, 7 aa, Mus musculus
TSRNFAK

SEQ ID NO: 1419 - protein, 7 aa, Mus musculus
TSRNFAY

SEQ ID NO: 1420 - protein, 14 aa, Mus musculus
TVFWYGNNYAGFAY

SEQ ID NO: 1421 - protein, 17 aa, Mus musculus
VAWYHQKPGQSPKALIY

SEQ ID NO: 1422 - protein, 17 aa, Mus musculus
VAWYQHKPGQSPKLLIY

SEQ ID NO: 1423 - protein, 17 aa, Mus musculus
VAWYQQEPGQSPKALIY

SEQ ID NO: 1424 - protein, 17 aa, Mus musculus
VAWYQQKPGQAPRLLIY

SEQ ID NO: 1425 - protein, 17 aa, Mus musculus
VAWYQQKPGQSPKALIY

SEQ ID NO: 1426 - protein, 17 aa, Mus musculus
VAWYQQKPGQSPKLLIH

SEQ ID NO: 1427 - protein, 17 aa, Mus musculus
VAWYQQKPGQSPKLLIY

SEQ ID NO: 1428 - protein, 17 aa, Mus musculus
VAWYQQKPGQSPKLPIY

SEQ ID NO: 1429 - protein, 17 aa, Mus musculus
VAWYQQKPGQSPKPLIY

SEQ ID NO: 1430 - protein, 16 aa, Mus musculus
VAYQQKPGQSPKALIY

SEQ ID NO: 1431 - protein, 16 aa, Mus musculus
VAYQQKPGQSPKALIY

SEQ ID NO: 1432 - protein, 16 aa, Mus musculus
VAYQQKPGQSPKPLIY

SEQ ID NO: 1433 - protein, 17 aa, Mus musculus
VEWYQQKQGKSPQLLVY

SEQ ID NO: 1434 - protein, 12 aa, Mus musculus
VHVRRGDQAGNQ

SEQ ID NO: 1435 - protein, 17 aa, Mus musculus
VLWYQQKPGQSPKLLIY

SEQ ID NO: 1436 - protein, 17 aa, Mus musculus
VNWYQQKPDGTVKLLIY

SEQ ID NO: 1437 - protein, 31 aa, Mus musculus
VPDRFAGSGSGTDFTLTISNVQSEDLAEYFC

SEQ ID NO: 1438 - protein, 31 aa, Mus musculus
VPDRFTGSGSGTDFTLTINNVQSEDLAEYFC

SEQ ID NO: 1439 - protein, 31 aa, Mus musculus
VPDRFTGSGSGTDFTLTISNVQSEDLAEYFC

SEQ ID NO: 1440 - protein, 31 aa, Mus musculus; identical to SEQ ID NO: 1439

SEQ ID NO: 1441 - protein, 10 aa, Mus musculus
VRRGDQAGNE

SEQ ID NO: 1442 - protein, 8 aa, Mus musculus
VRSPILDY

SEQ ID NO: 1443 - protein, 7 aa, Mus musculus
VRVTPAS

SEQ ID NO: 1444 - protein, 7 aa, Mus musculus
VSRNFAK

SEQ ID NO: 1445 - protein, 7 aa, Mus musculus
VSRNFAN

SEQ ID NO: 1446 - protein, 17 aa, Mus musculus
VSWYQQKPEQSPKLLIY

SEQ ID NO: 1447 - protein, 17 aa, Mus musculus
VVWYQQKPGQSPKLLIY

SEQ ID NO: 1448 - protein, 12 aa, Mus musculus
VWGAGTTVTVSS

SEQ ID NO: 1449 - protein, 17 aa, Mus musculus
  VARIANT 2: note = Xaa can be any naturally occurring amino acid
VXWYQQKPGQSPKALIY

SEQ ID NO: 1450 - skipped

SEQ ID NO: 1451 - protein, 11 aa, Mus musculus
WGQGATVTVSS

SEQ ID NO: 1452 - protein, 11 aa, Mus musculus
WGQGTTLTVSA

SEQ ID NO: 1453 - protein, 9 aa, Mus musculus
WGQGTTVSA

SEQ ID NO: 1454 - protein, 11 aa, Mus musculus
WGRGTSLTVSS

SEQ ID NO: 1455 - protein, 11 aa, Mus musculus
WGRGTTLTVSS

SEQ ID NO: 1456 - protein, 17 aa, Mus musculus
WIGWIKQRPGHGLEWIG

SEQ ID NO: 1457 - protein, 17 aa, Mus musculus
WIGWVKQRPGHDLEWIG

SEQ ID NO: 1458 - protein, 17 aa, Mus musculus
WIGWVKQRPGHGLEWIG

SEQ ID NO: 1459 - protein, 17 aa, Mus musculus
WIGWVKQRPGRGLEWIG

SEQ ID NO: 1460 - protein, 17 aa, Mus musculus
WIGWVKQRSGHGLEWIG

SEQ ID NO: 1461 - protein, 9 aa, Mus musculus
WQGAHFPWT

SEQ ID NO: 1462 - protein, 8 aa, Mus musculus
WQGTFSSH

SEQ ID NO: 1463 - protein, 9 aa, Mus musculus
WQGTHFPQT

SEQ ID NO: 1464 - protein, 9 aa, Mus musculus
WQGTHFPRT

SEQ ID NO: 1465 - protein, 9 aa, Mus musculus
WQGTHFPWT

SEQ ID NO: 1466 - protein, 8 aa, Mus musculus
WQGTHFRT

SEQ ID NO: 1467 - protein, 12 aa, Mus musculus
WTFGGGTKLEIK

SEQ ID NO: 1468 - protein, 12 aa, Mus musculus
WTFGGGTKLEMK

SEQ ID NO: 1469 - protein, 12 aa, Mus musculus
WTFGRGTKLEIK

SEQ ID NO: 1470 - protein, 15 aa, Mus musculus
WYQQKPGQAPRLLIY

SEQ ID NO: 1471 - protein, 15 aa, Mus musculus
WYQQKPGQSPKALIY

SEQ ID NO: 1472 - protein, 15 aa, Mus musculus
WYQQKPGQSPKALIY

SEQ ID NO: 1473 - protein, 15 aa, Mus musculus
WYQQKPGQSPKPLIY

SEQ ID NO: 1474 - protein, 11 aa, Mus musculus
  VARIANT 1: note = Xaa can be any naturally occurring amino acid
  VARIANT 6: note = Xaa can be any naturally occurring amino acid
XGRGTXLTVSS

SEQ ID NO: 1475 - protein, 9 aa, Mus musculus
  VARIANT 1: note = Xaa can be any naturally occurring amino acid
XQYNSYPYT

SEQ ID NO: 1476 - skipped

SEQ ID NO: 1477 - protein, 36 aa, Mus musculus
YRYGGVPDRFTGSGSGTDFTLTISNVQSEDLAEYFC

SEQ ID NO: 1478 - protein, 36 aa, Mus musculus
YRYNGVPDRFTGSGSGTDFTLTISNVQSEDLAEYFC

SEQ ID NO: 1479 - protein, 36 aa, Mus musculus
YRYSGVPDRFTGSGSGTDFTLKISRVEAEDLGVYFC

SEQ ID NO: 1480 - protein, 36 aa, Mus musculus
YRYSGVPDRFTGSGSGTDFTLTINNVQSEDLAEYFC

SEQ ID NO: 1481 - protein, 36 aa, Mus musculus
YRYSGVPDRFTGSGSGTDFTLTINSVQSEDLADYFC

SEQ ID NO: 1482 - protein, 36 aa, Mus musculus
YRYSGVPDRFTGSGSGTDFTLTISNVQSEDLADYFC

SEQ ID NO: 1483 - protein, 36 aa, Mus musculus
YRYSGVPDRFTGSGSGTDFTLTISNVQSEDLAEYFC

SEQ ID NO: 1484 - protein, 36 aa, Mus musculus; identical to SEQ ID NO: 1483

SEQ ID NO: 1485 - protein, 36 aa, Mus musculus
YRYSGVPDRFTGSGSGTDFTLTISNVQSEDLAEYLC

SEQ ID NO: 1486 - protein, 36 aa, Mus musculus
YRYSGVPDRFTGSGSGTDFTLTISNVQSEDLTEYFC

SEQ ID NO: 1487 - protein, 36 aa, Mus musculus
YRYSGVPDRFTGSGSGTGFTLTISNVQSEDLAEYFC

SEQ ID NO: 1488 - protein, 36 aa, Mus musculus
YRYSGVPDRITGRGSGTDFTLTISNVQSEDLADYFC

SEQ ID NO: 1489 - protein, 36 aa, Mus musculus
YRYSGVPDRLTGSGSGTDFTLTISNVQSEDLAEYFC

SEQ ID NO: 1490 - protein, 36 aa, Mus musculus
YRYSGVPGRFTGSGSGTDFTLTISNVQSEDLAEYFC

SEQ ID NO: 1491 - protein, 36 aa, Mus musculus
YRYTGVPDRFTGSGSGTDFTFTISSVQAEDLAVYYC

SEQ ID NO: 1492 - protein, 36 aa, Mus musculus
YRYTGVPDRFTGSGSRTDFTFTISSVQAEDLAVYYC

SEQ ID NO: 1493 - protein, 12 aa, Mus musculus
YTFGGGTKLEIK

SEQ ID NO: 1494 - protein, 12 aa, Mus musculus
YTFGGGTKLEMK

SEQ ID NO: 1495 - skipped

SEQ ID NO: 1496 - protein, 12 aa, Mus musculus
YWGQGTLVTALS

SEQ ID NO: 1497 - DNA, 20 bp, synthetic construct
  misc_feature 1..20: note = sequencing primer
  source 1..20: mol_type = other DNA
acgcctgcgaagtcacccat

SEQ ID NO: 1498 - DNA, 31 bp, synthetic construct
  misc_feature 1..31: note = sequencing primer
  source 1..31: mol_type = other DNA
agaaacacaaagtctacgcctgcgaagtcac

SEQ ID NO: 1499 - DNA, 24 bp, synthetic construct
  misc_feature 1..24: note = sequencing primer
  source 1..24: mol_type = other DNA
agcggataacaatttcacacagga

SEQ ID NO: 1500 - DNA, 20 bp, synthetic construct
  misc_feature 1..20: note = sequencing primer
  source 1..20: mol_type = other DNA
cggataacaatttcacacag

SEQ ID NO: 1501 - protein, 118 aa, Mus musculus
DVKLVESGGLVKPGGSLKLSCAASGFTFSSYAMSWVRQTPEKRLEWVASISSGGSTYYPDSVKGRFTISRDNARNILYLQMSSLRSEDTAMYYCARGEIWGKAWFAYWGQGTLVTVSA